(19) United States
(12) Patent Application Publication
(10) Pub. No.: US 2024/0269137 A1
(43) Pub. Date: Aug. 15, 2024

(54) HDAC6 INHIBITORS FOR TREATMENT OF DILATED CARDIOMYOPATHY

(71) Applicant: Tenaya Therapeutics, Inc., South San Francisco, CA (US)

(72) Inventors: Mohammad A. MANDEGAR, South San Francisco, CA (US); Jin YANG, South San Francisco, CA (US); Timothy C. HOEY, Hillsborough, CA (US)

(21) Appl. No.: 18/556,657

(22) PCT Filed: Apr. 22, 2022
(86) PCT No.: PCT/US22/26065

§ 371 Date: Oct. 20, 2023

Related U.S. Application Data
(60) Provisional application No. 63/178,901, filed on Apr. 23, 2021.

(51) Int. Cl. A61K 31/505; A61K 31/17; A61K 31/42; A61K 31/4245; A61K 31/427; A61K 31/437; A61P 9/04; G01N 33/50

(52) U.S. Cl. CPC A61K 31/505; A61K 31/17; A61K 31/42; A61K 31/4245; A61K 31/427; A61K 31/437; A61P 9/04; G01N 33/5008; G01N 2800/325

(57) ABSTRACT

Provided herein are methods of treating or preventing dilated cardiomyopathy (DCM) with an HDAC6 inhibitor. A variety of HDAC6 inhibitors are described herein for use in treating or preventing DCM. In one aspect, described herein are methods of treating a human patient by orally administering an HDAC6 inhibitor, such as an inhibitor of Formula (I) or Formula (II). In one aspect, described herein are methods of treating a human patient with DCM associated with a reduced ejection fraction.

### CROSS-REFERENCE TO RELATED APPLICATION

This application claims the benefit of and priority to U.S. Provisional Patent Application No. 63/178,901, filed Apr. 23, 2021, which is incorporated herein by reference in its entirety.

### FIELD

The present disclosure relates to treatment of dilated cardiomyopathy (DCM).

### BACKGROUND

Dilated cardiomyopathy (DCM) is a form of heart muscle weakness characterized by reduced cardiac output, as well as thinning and enlargement of left ventricular chambers (McNally et al., 2013; Villard et al., 2011). DCM affects approximately 1/2500 adults (Villard et al., 2011), accounts for 30% to 40% of all heart failure cases in clinical trials, and is a major cause of heart transplants (Everly, 2008; Haas et al., 2015).

Current treatments for heart failure include angiotensin-converting enzyme inhibitors, angiotensin receptor blockers, beta-blockers, aldosterone antagonists, vasodilators, angiotensin receptor-neprilysin inhibitors, and sodium-glucose cotransporter 2 inhibitors. These treatments mainly ameliorate symptoms and do not target the underlying molecular mechanisms associated with genetic forms of heart failure (Cleland et al., 2020).

Approximately one-third of individuals with DCM have an inherited form of the disease. Familial DCM accounts for 30% to 50% of all DCM cases and has an autosomal-dominant mode of inheritance (Haas et al., 2015). Genetic forms of DCM have been associated with more than 50 genes, and over 50% of patients with DCM have at least one mutation in one of these genes (Haas et al., 2015; McNally et al., 2013). Several of these DCM-associated genes code for central regulators of protein quality control, and mutations in these genes lead to protein aggregation and accumulation of misfolded proteins (Fang et al., 2017; Stürner and Behl, 2017).

There is an unmet need for treatments for DCM.

### SUMMARY

The present disclosure relates generally to methods of treating dilated cardiomyopathy by administering an HDAC6 inhibitor, such as TYA-018 or an analogue thereof.

In one aspect, the disclosure provides method of treating or preventing dilated cardiomyopathy in a subject in need thereof, comprising administering a therapeutically effective amount of a HDAC6 inhibitor.

In some embodiments, the disclosure provides methods of treating or preventing dilated cardiomyopathy associated with a decreased ejection fraction in a subject in need thereof, comprising administering a therapeutically effective amount of a HDAC6 inhibitor.

In some embodiments, the disclosure provides methods of treating dilated cardiomyopathy in a subject in need thereof, comprising administering an HDAC6 inhibitor, wherein the HDAC6 inhibitor is fluoroalkyl-oxadiazole derivative. In some embodiments, the HDAC6 inhibitor is fluoroalkyl-oxadiazole derivative according to the following Formula:

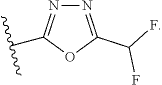 [SMILES: *c1nnc(C(F)F)o1]

In some embodiments, the disclosure provides methods of preventing dilated cardiomyopathy in a subject in need thereof, comprising administering an HDAC6 inhibitor, wherein the HDAC6 inhibitor is fluoroalkyl-oxadiazole derivative. In some embodiments, the HDAC6 inhibitor is fluoroalkyl-oxadiazole derivative according to the following Formula:

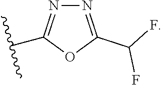 [SMILES: *c1nnc(C(F)F)o1]

In some embodiments, the HDAC6 inhibitor is a compound according to Formula (I):

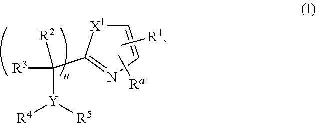

wherein
R¹ is selected from the group consisting of:

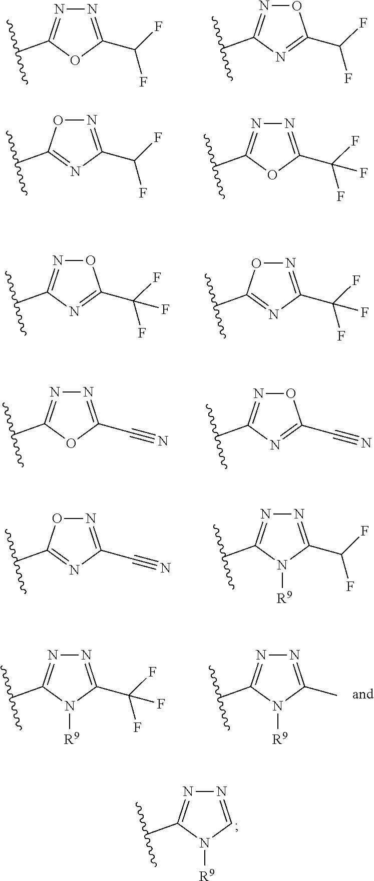

R^{a} is selected from the group consisting of H, halo, C₁₋₃ alkyl, cycloalkyl, haloalkyl, and alkoxy;
R² and R³ are independently selected from the group consisting of H, halogen, alkoxy, haloalkyl, aryl, heteroaryl, alkyl, and cycloalkyl each of which is optionally substituted, or R² and R³ together with the atom to which they are attached form a cycloalkyl or heterocyclyl;
R⁴ and R⁵ are independently selected from the group consisting of H, —(SO₂)R², —(SO₂)NR²R³, —(CO)R², —(CONR²R³), aryl, arylheteroaryl, alkylenearyl, heteroaryl, cycloalkyl, heterocyclyl, alkyl, haloalkyl, and alkoxy, each of which is optionally substituted, or R⁴ and R⁵ together with the atom to which they are attached form a cycloalkyl or heterocyclyl, each of which is optionally substituted;
R⁹ is selected from the group consisting of H, C₁-C₆ alkyl, haloalkyl, cycloalkyl and heterocyclyl;
X¹ is selected from the group consisting of S, O, NH and NR⁶, wherein R⁶ is selected from the group consisting of C₁-C₆ alkyl, alkoxy, haloalkyl, cycloalkyl and heterocyclyl;
Y is selected from the group consisting of CR², O, N, S, SO, and SO₂, wherein when Y is O, S, SO, or SO₂, R⁵ is not present and when R⁴ and R⁵ together with the atom to which they are attached form a cycloalkyl or heterocyclyl, Y is CR² or N; and
n is selected from 0, 1, and 2.

In some embodiments, the HDAC6 inhibitor is a compound according to Formula (Ik):

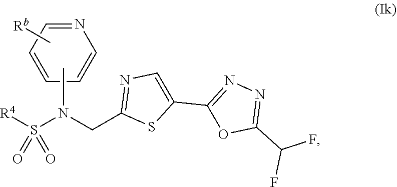

or a pharmaceutically acceptable salt thereof wherein:
R^{b} is H, halogen, alkyl, cycloalkyl, —CN, haloalkyl, or haloalkoxy; and
R⁴ is alkyl, alkoxy, haloalkyl, or cycloalkyl, each of which is optionally substituted.

In some embodiments of Formula (Ik), R^{b} is H, halogen, haloalkyl, or haloalkoxy.

In some embodiments of Formula (Ik), R⁴ is optionally substituted alkyl or cycloalkyl.

In some embodiments, the HDAC6 inhibitor is a compound according to Formula (Ik-1):

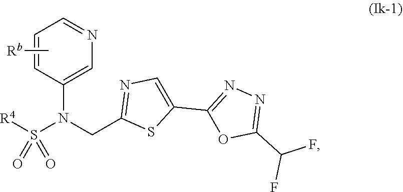

or a pharmaceutically acceptable salt thereof,
   wherein:
R^{b} is H, halogen, alkyl, cycloalkyl, —CN, haloalkyl, or haloalkoxy; and
R⁴ is alkyl, alkoxy, haloalkyl, or cycloalkyl, each of which is optionally substituted.

In some embodiments of Formula (Ik-1), R^{b} is H, halogen, haloalkyl, or haloalkoxy.

In some embodiments of Formula (Ik-1), R⁴ is optionally substituted alkyl or cycloalkyl.

In some embodiments of Formula (Ik-1), R⁴ is alkyl.

In some embodiments, the HDAC6 inhibitor is a compound according to Formula (Ik-2):

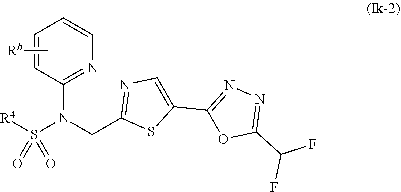

or a pharmaceutically acceptable salt thereof,
   wherein:
R^{b} is H, halogen, alkyl, cycloalkyl, —CN, haloalkyl, or haloalkoxy; and
R⁴ is alkyl, alkoxy, haloalkyl, or cycloalkyl, each of which is optionally substituted.

In some embodiments of Formula (Ik-2), R^{b} is H, halogen, haloalkyl, or haloalkoxy.

In some embodiments of Formula (Ik-2), R⁴ is optionally substituted alkyl.

In some embodiments, the HDAC6 inhibitor is a compound is a compound according to Formula I(y):

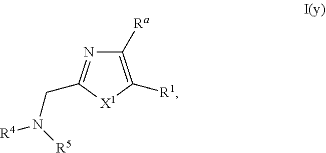

or a pharmaceutically acceptable salt thereof, wherein:
X¹ is S;
R^{a} is selected from the group consisting of H, halogen, and C₁₋₃ alkyl;
R¹ is

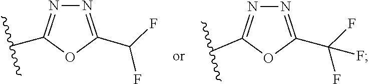 [SMILES: *c1nnc(C(F)F)o1 ; *c1nnc(C(F)(F)F)o1]

R² is selected from the group consisting of alkyl, alkoxy, and cycloalkyl, each of which is optionally substituted;
R³ is H or alkyl;
R⁴ is selected from the group consisting of alkyl, —(SO₂)R², —(SO₂)NR²R³, and —(CO)R²; and
R⁵ is aryl or heteroaryl; or R⁴ and R⁵ together with the atom to which they are attached form a heterocyclyl, each of which is optionally substituted.

In some embodiments of Formula I(y), R^{a} is H.

In some embodiments of Formula I(y), R¹ is

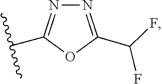 [SMILES: *c1nnc(C(F)F)o1]

In some embodiments of Formula I(y), R⁴ is —(SO₂)R².

In some embodiments of Formula I(y), —(SO₂)R² is —(SO₂)alkyl, —(SO₂)alkyleneheterocyclyl, —(SO₂)haloalkyl, —(SO₂)haloalkoxy, or —(SO₂)cycloalkyl.

In some embodiments of Formula I(y), R⁵ is heteroaryl.

In some embodiments of Formula I(y), the heteroaryl is a 5- to 6-membered heteroaryl.

In some embodiments of Formula I(y), the 5- to 6-membered heteroaryl is selected from the group consisting of

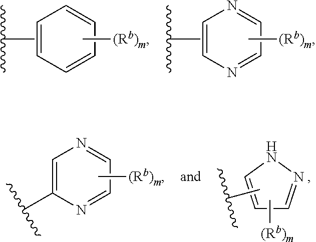

wherein R is halogen, alkyl, alkoxy, cycloalkyl, —CN, haloalkyl, or haloalkoxy; and m is 0 or 1.

In some embodiments of Formula I(y), R^{b} is F, Cl, —CH₃, —CH₂CH₃, —CF₃, —CHF₂, —CF₂CH₃, —CN, —OCH₃, —OCH₂CH₃, —OCH(CH₃)₂, —OCF₃, —OCHF₂, —OCH₂CF₂H, and cyclopropyl.

In some embodiments of Formula I(y), the aryl is selected from the group consisting of phenyl, 3-chlorophenyl, 3-chloro-4-fluorophenyl, 3-trifluoromethylphenyl, 3,4-difluorophenyl, and 2,6-difluorophenyl.

In some embodiments of Formula I(y), the compound is:

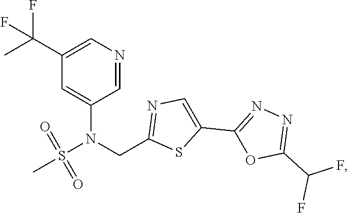 [SMILES: CC(F)(F)c1cncc(N(Cc2ncc(-c3nnc(C(F)F)o3)s2)S(C)(=O)=O)c1]

or a pharmaceutically acceptable salt thereof.

In some embodiments of Formula I(y), the compound is:

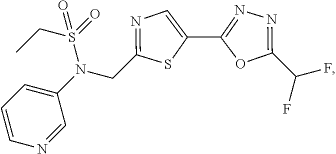 [SMILES: CCS(=O)(=O)N(Cc1ncc(-c2nnc(C(F)F)o2)s1)c1cccnc1]

or a pharmaceutically acceptable salt thereof.

In some embodiments of Formula I(y), the compound is:

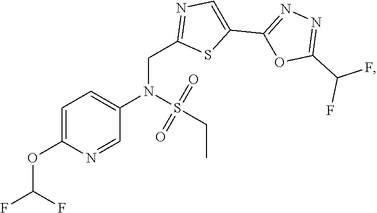 [SMILES: CCS(=O)(=O)N(Cc1ncc(-c2nnc(C(F)F)o2)s1)c1ccc(OC(F)F)nc1]

or a pharmaceutically acceptable salt thereof.

In some embodiments of Formula I(y), the compound is:

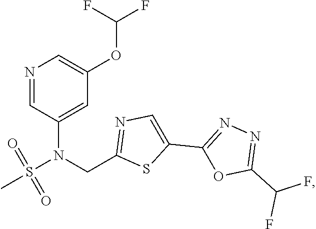 [SMILES: CS(=O)(=O)N(Cc1ncc(-c2nnc(C(F)F)o2)s1)c1cncc(OC(F)F)c1]

or a pharmaceutically acceptable salt thereof.

In some embodiments of Formula I(y), the compound is:

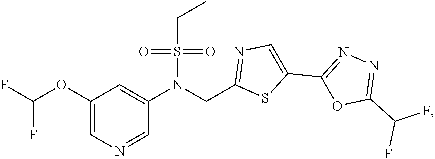 [SMILES: CCS(=O)(=O)N(Cc1ncc(-c2nnc(C(F)F)o2)s1)c1cncc(OC(F)F)c1]

or a pharmaceutically acceptable salt thereof.

In some embodiments of Formula I(y), the compound is:

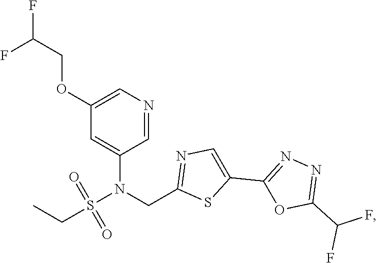 [SMILES: CCS(=O)(=O)N(Cc1ncc(-c2nnc(C(F)F)o2)s1)c1cncc(OCC(F)F)c1]

or a pharmaceutically acceptable salt thereof.

In some embodiments of Formula I(y), the compound is:

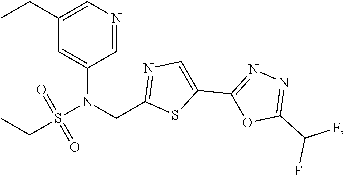 [SMILES: CCc1cncc(N(Cc2ncc(-c3nnc(C(F)F)o3)s2)S(=O)(=O)CC)c1]

or a pharmaceutically acceptable salt thereof.

In some embodiments of Formula I(y), the compound is:

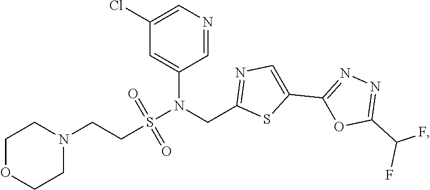 [SMILES: O=S(=O)(CCN1CCOCC1)N(Cc1ncc(-c2nnc(C(F)F)o2)s1)c1cncc(Cl)c1]

or a pharmaceutically acceptable salt thereof.

In some embodiments of Formula I(y), the compound is:

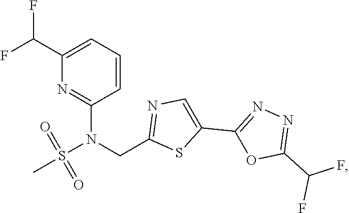 [SMILES: CS(=O)(=O)N(Cc1ncc(-c2nnc(C(F)F)o2)s1)c1cccc(C(F)F)n1]

or a pharmaceutically acceptable salt thereof.

In some embodiments of Formula I(y), the compound is:

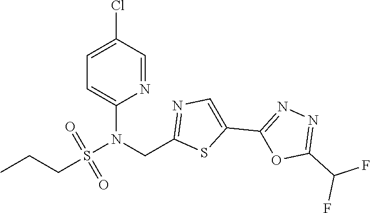 [SMILES: CCCS(=O)(=O)N(Cc1ncc(-c2nnc(C(F)F)o2)s1)c1ccc(Cl)cn1]

or a pharmaceutically acceptable salt thereof.

In some embodiments, the HDAC6 inhibitor is selected from the group consisting of:

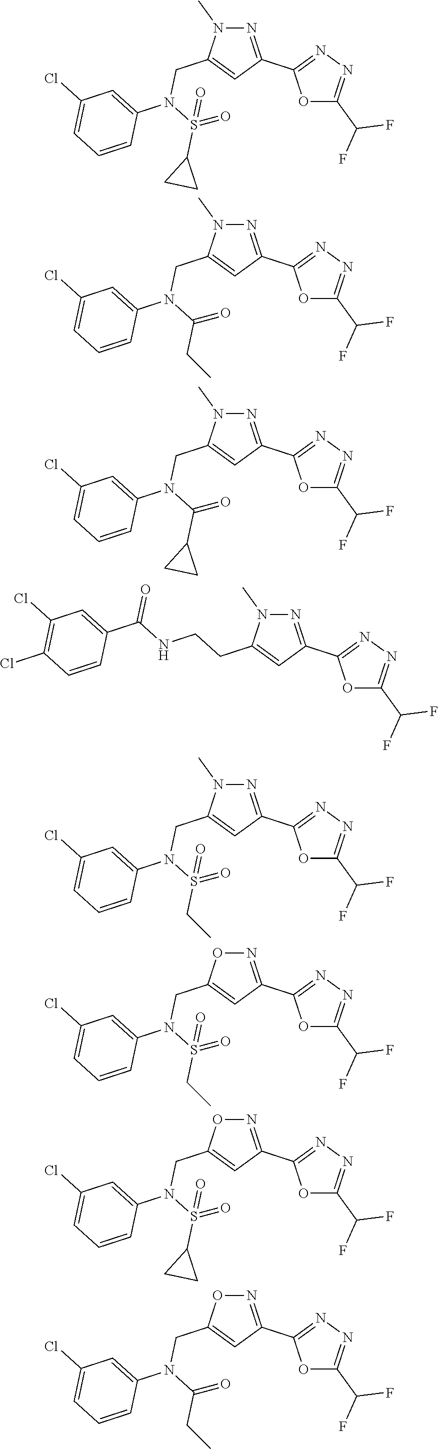

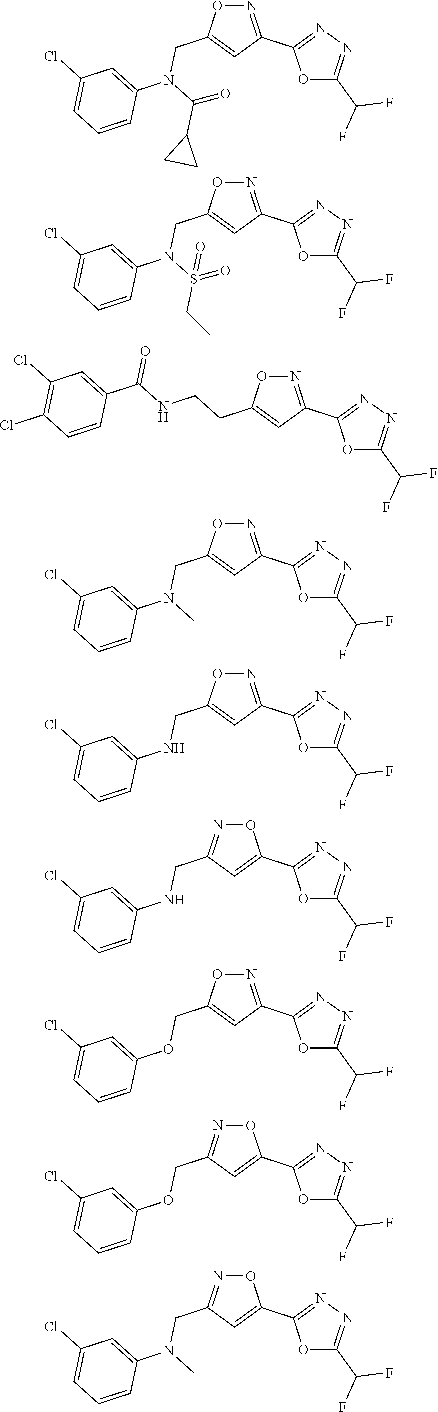

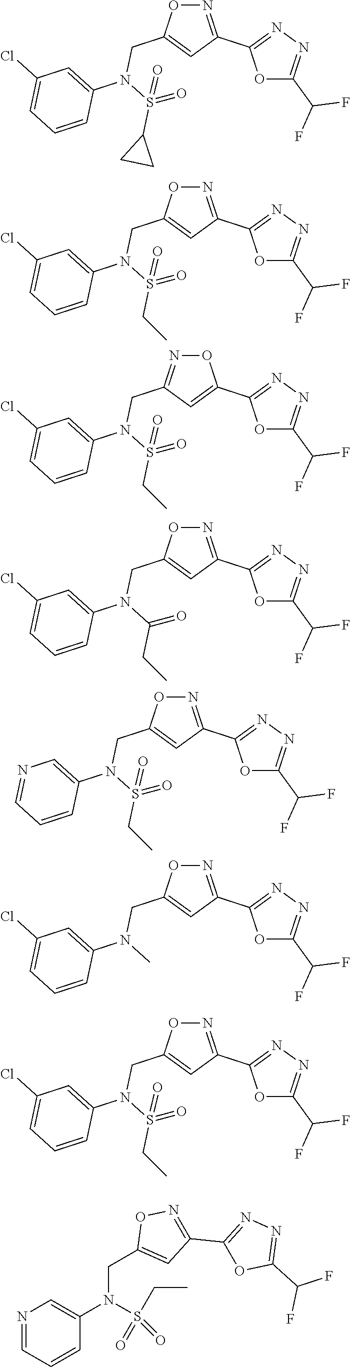

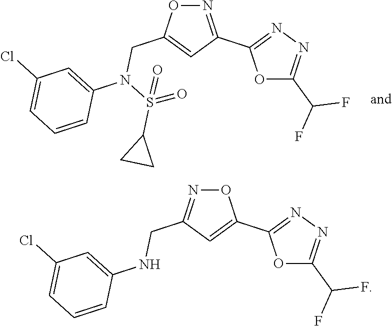

In some embodiments, the HDAC6 inhibitor is

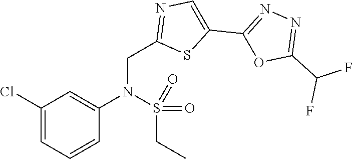 [SMILES: CCS(=O)(=O)N(Cc1ncc(-c2nnc(C(F)F)o2)s1)c1cccc(Cl)c1]

(TYA-018) or an analog thereof.

In some embodiments, the HDAC6 inhibitor is TYA-018.

In some embodiments, the HDAC6 inhibitor is a compound of Formula (II):

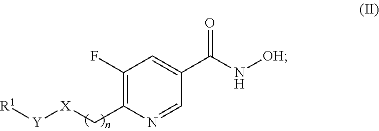

wherein
n is 0 or 1;
X is O, NR⁴, or CR⁴R^{4′};
Y is a bond, CR²R³ or S(O)₂;
R¹ is selected from the group consisting of H, amido, carbocyclyl, heterocyclyl, aryl, and heteroaryl;
R² and R³ are independently selected from the group consisting of H, halogen, alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or
R¹ and R² taken together with the carbon atom to which they are attached form a carbocyclyl or heterocyclyl; or
R² and R³ taken together with the carbon atom to which they are attached form a carbocyclyl or heterocyclyl; and
R⁴ and R^{4′} are each independently selected from the group consisting of H, alkyl, —CO₂-alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or
R⁴ and R^{4″} taken together with the carbon atom to which they are attached form a carbocyclyl or heterocyclyl;
wherein each alkyl, carbocyclyl, heterocyclyl, aryl, and heteroaryl is independently optionally substituted with one or more substituents selected from the group consisting of halogen, haloalkyl, oxo, hydroxy, alkoxy, —OCH₃, —CO₂CH₃, —C(O)NH(OH), —CH₃, morpholine, and —C(O)N-cyclopropyl.

In some embodiments, the HDAC6 inhibitor is CAY10603, tubacin, rocilinostat (ACY-1215), citarinostat (ACY-241), ACY-738, QTX-125, CKD-506, nexturastat A, tubastatin A, or HPOB.

In some embodiments, the HDAC6 inhibitor is tubastatin A.

In some embodiments, the HDAC6 inhibitor is ricolinostat.

In some embodiments, the HDAC6 inhibitor is CAY10603.

In some embodiments, the HDAC6 inhibitor is nexturastat A.

In some embodiments, the HDAC6 inhibitor is at least 100-fold selective against HDAC6 compared to all other isozymes of HDAC.

In some embodiments, the HDAC6 inhibitor reduces HDAC6 activity by at least 30%, at least 40%, at least 50%, at least 60%, at least 70%, at least 75%, at least 80%, at least 90%, at least 95%, or at least 98%. In some embodiments, the HDAC6 inhibitor substantially eliminates HDAC6 activity.

In one aspect, the disclosure provides methods of treating or preventing dilated cardiomyopathy in a subject in need thereof, comprising administering a gene silencing agent, such as an RNA silencing agent (e.g., siRNA).

In some embodiments, the dilated cardiomyopathy is familial dilated cardiomyopathy.

In some embodiments, the dilated cardiomyopathy is dilated cardiomyopathy due to one or more BLC2-Associated Athanogene 3 (BAG3) mutations.

In some embodiments, the subject has a deleterious mutation in the BAG3 gene. In some embodiments, the subject has BAG3^{E455K} mutation.

In some embodiments, the dilated cardiomyopathy is dilated cardiomyopathy due to one or more muscle LIM protein (MLP) mutations.

In some embodiments, the subject has a deleterious mutation in the CSPR3 gene encoding MLP.

In some embodiments, the subject is a human.

In some embodiments, the administering to the subject is oral.

In some embodiments, the method restores the ejection fraction of the subject to at least about the ejection fraction of a subject without dilated cardiomyopathy.

In some embodiments, the method increases the ejection fraction of the subject compared to the subject's ejection fraction before treatment.

In some embodiments, the method restores the ejection fraction of the subject to at least about 20%, at least about 20%, at least about 30%, at least about 40%, or at least about 50%.

In some embodiments, the method increase the ejection fraction of the subject to by at least about 5%, at least about 10%, at least about 20%, at least about 30%, or at least about 40%.

In some embodiments, the method reduces HDAC6 activity in the heart of the subject. In some embodiments, the method substantially eliminates HDAC6 activity in the heart of the subject.

In some embodiments, the method prevents heart failure in the subject.

In some embodiments, the method reduces left ventricular internal diameter at diastole (LVIDd) in the subject.

In some embodiments, the method reduces left ventricular internal diameter at systole (LVIDs) in the subject.

In some embodiments, the method reduces left ventricular mass in the subject.

In some embodiments, the method comprises selecting the HDAC6 inhibitor by performing in vitro testing for selective inhibition of HDAC6 on each member of the plurality of candidate compounds, thereby identifying a selected compound for use as the HDAC6 inhibitor.

In another aspect, the disclosure provides an HDAC6 inhibitor for use in a method for treating dilated cardiomyopathy.

In another aspect, the disclosure provides a pharmaceutical composition for use in a method for treating dilated cardiomyopathy, comprising an HDAC6 inhibitor.

In another aspect, the disclosure provides a kit, comprising an HDAC6 inhibitor and instructions for use in a method for treating dilated cardiomyopathy.

In another aspect, the disclosure provides use of an HDAC6 inhibitor in treating dilated cardiomyopathy.

In another aspect, the disclosure provides a method of identifying a compound for treatment of dilated cardiomyopathy, comprising contacting a cell culture comprising cells having an inactivating mutation in BAG3 with each member of a plurality of candidate compounds; and selecting a compound that reduces sarcomere damage in the cells.

In another aspect, the disclosure provides method of treating dilated cardiomyopathy in a subject in need thereof, comprising: (a) identifying a compound by contacting a cell culture comprising cells having an inactivating mutation in BAG3 with each member of a plurality of candidate compounds; and selecting a compound which reduces sarcomere damage; and (b) administering a therapeutically effective amount of the selected compound to the subject.

### BRIEF DESCRIPTION OF DRAWINGS

FIG. 1 shows a graphic abstract of the experimental method used herein.

FIG. 2A shows protein quantification using immunostaining of iPSC-CMs treated with SCR and BAG3 siRNA. BAG3 protein levels were reduced by approximately 75%. Also, protein levels of MYBPC3 and p62 were reduced, suggesting defects in sarcomere and autophagic flux. Error bars=SD. ****P<0.0001.

FIG. 2B shows a graphic abstract of the experimental method used herein.

FIG. 2C shows quantification of sarcomere damage in iPSC-CMs treated with SCR or BAG3 siRNA. The number of damaged iPSC-CMs increased as a function of time in BAG3 knockdown (KD) cells. Error bars=SD. ****P<0.0001.

FIG. 3A shows a schematic of the high-content screening approach using iPSC-CMs.

FIG. 3B shows an unbiased screen was performed using a library of 5500 bioactive compounds. iPSC-CMs were treated with BAG3 siRNA and compounds at a concentration of 1 μM. Hits were first identified using deep learning on control iPSC-CMs treated with either SCR or BAG3 siRNA. The hit threshold was set at a cardiomyocyte score of 0.3.

FIG. 3C shows the top 24 compounds consisted of histone deacetylase (HDAC) and microtubule inhibitors. In addition, three known heart failure agents were identified: sotalol (beta-blocker and K-channel blocker), omecamtiv mecarbil (cardiac myosin activator), and anagrelide (PDE3 inhibitor).

FIG. 4A shows iPSC-CMs were treated with a panel of pan- and isozyme-specific HDAC inhibitors, and protein levels were quantified 4 days after treatment using immunochemistry at 5 doses ranging from 10 nM to 1000 nM. BAG3 protein levels did not increase in cells treated with HDAC inhibitors. Bortezomib (known to increase BAG3 expression) was used as a positive control.

FIG. 4B shows the same panel of HDAC inhibitors were used at 100 nM concentration, and RNA expression was quantified using qPCR 2 days after drug treatment. HDAC inhibitors did not activate BAG3 expression at the transcription level. Bortezomib (known to increase BAG3 expression) was used as a positive control. Error bars=SD.

FIGS. 5A-5C show target validation studies show inhibiting HDAC6 is sufficient to protect against sarcomere damage in BAG3-deficient iPSC-CMs

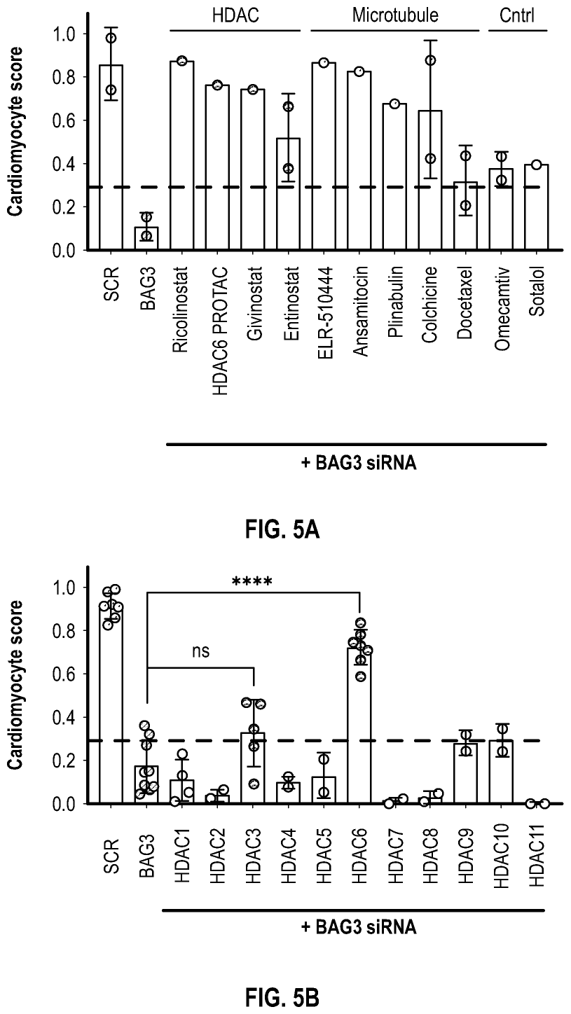

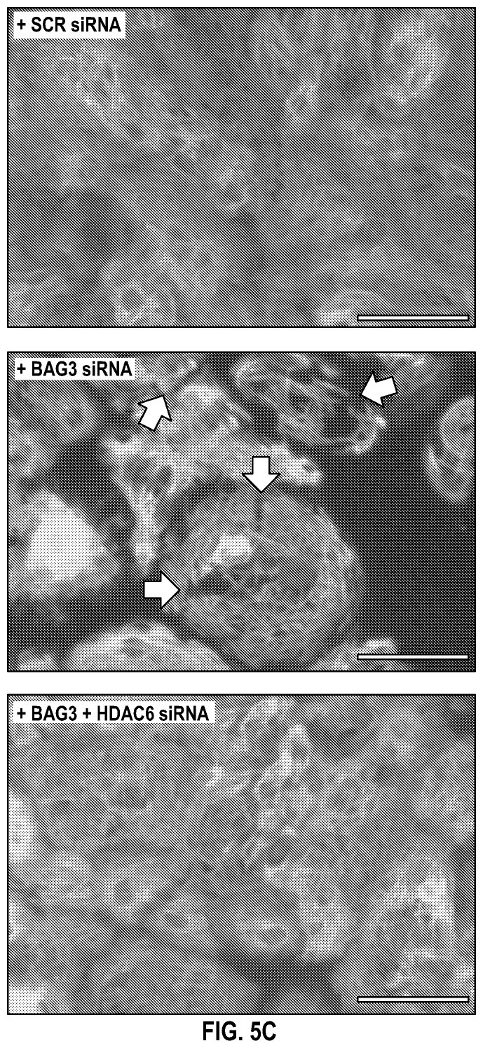

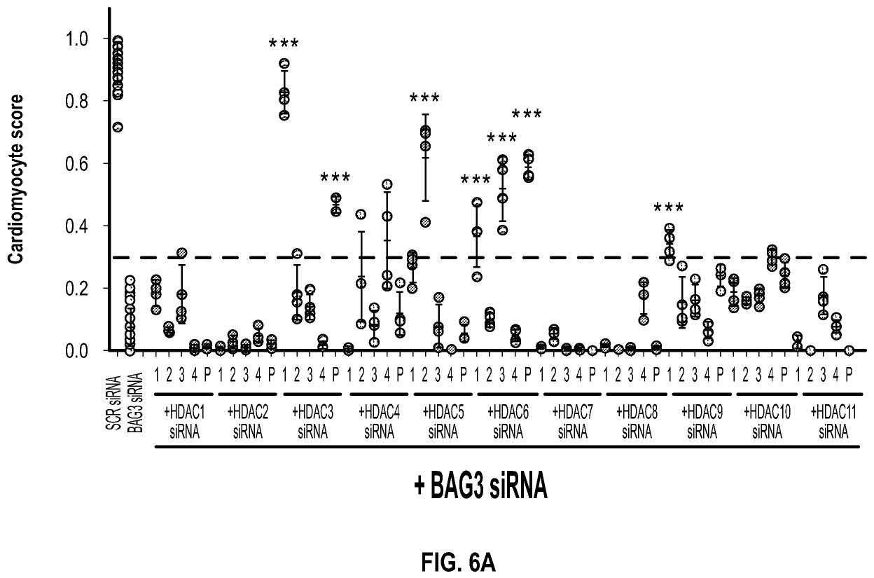

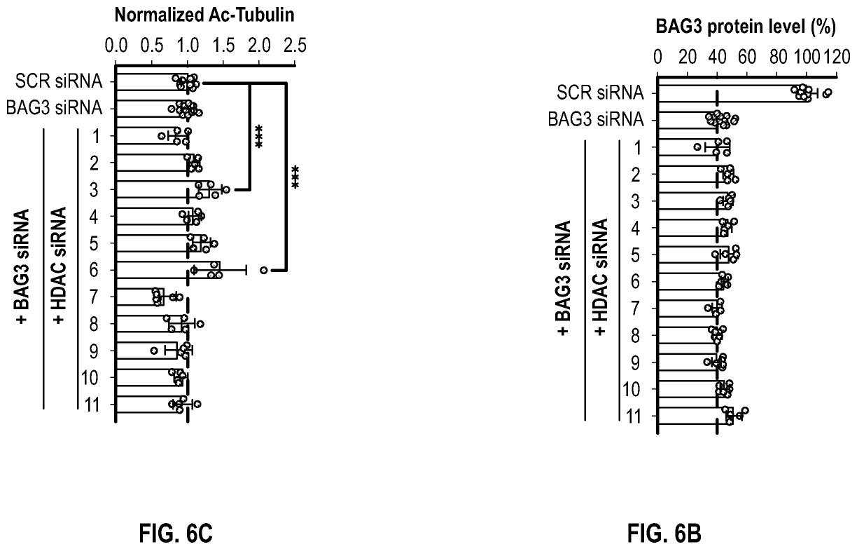

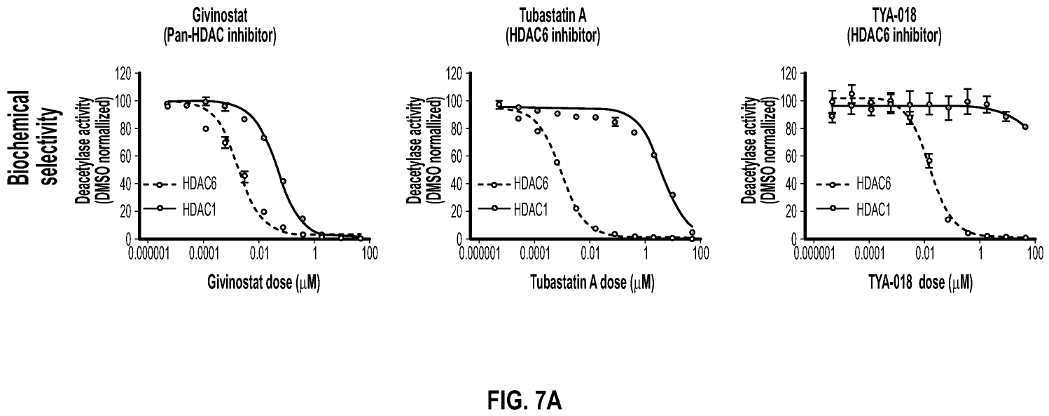

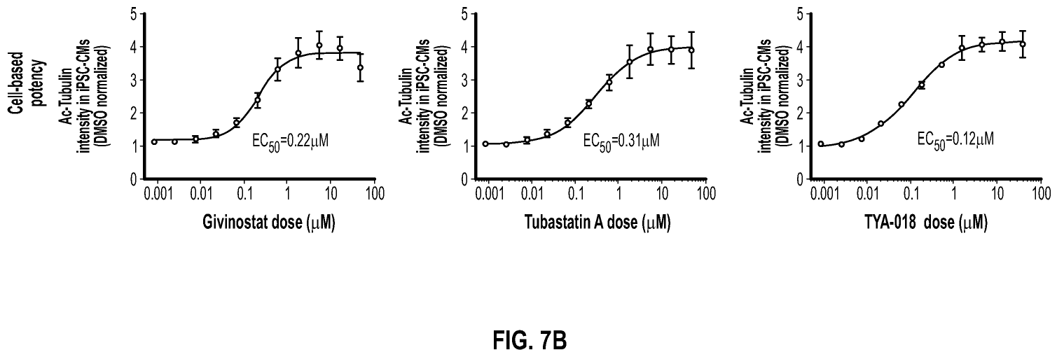

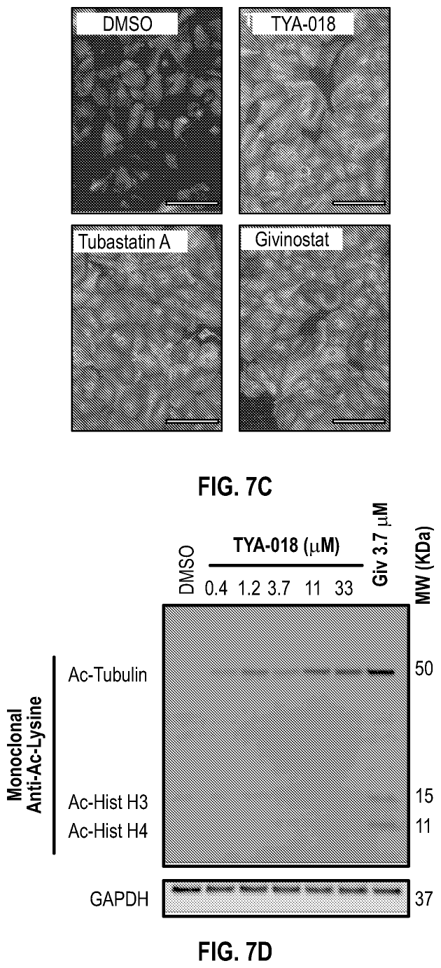

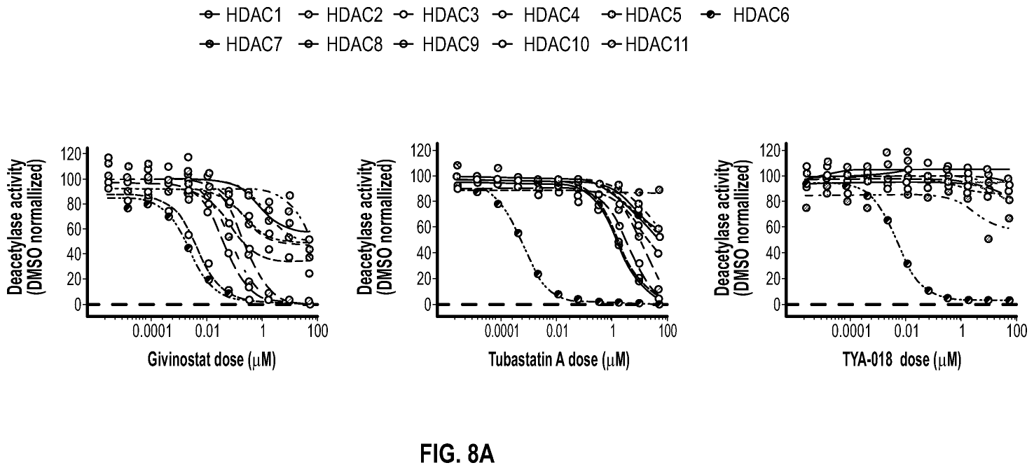

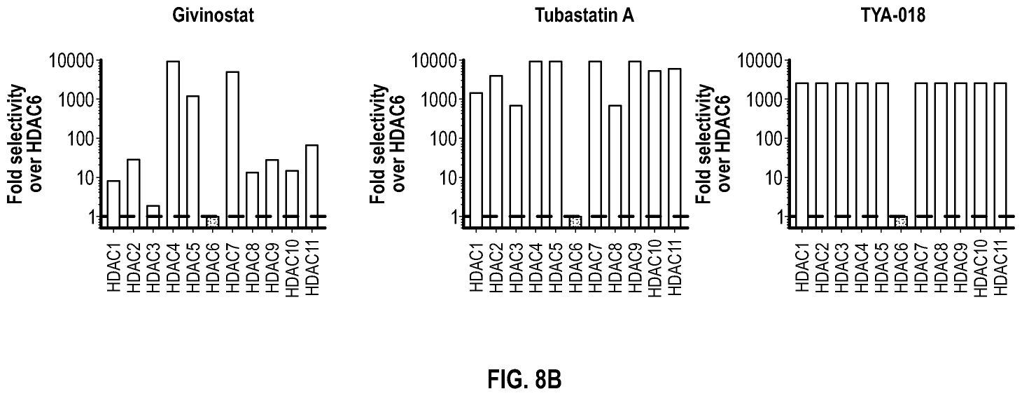

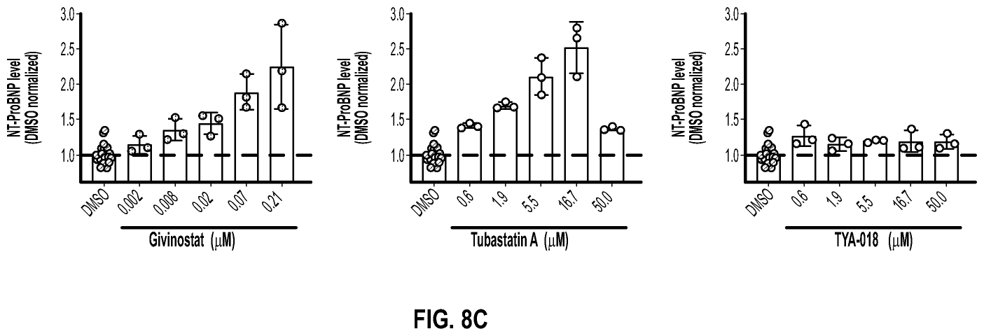

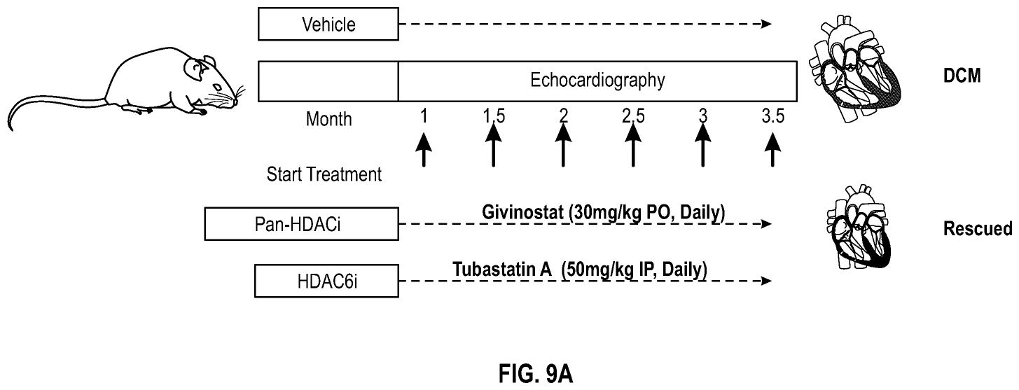

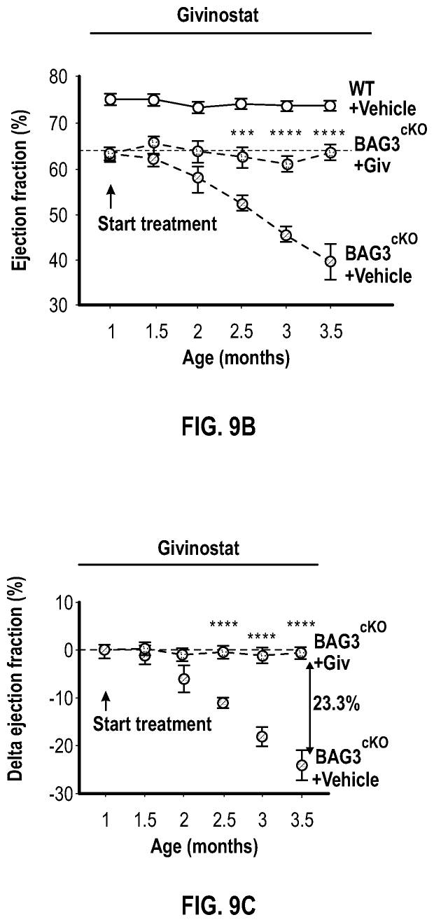

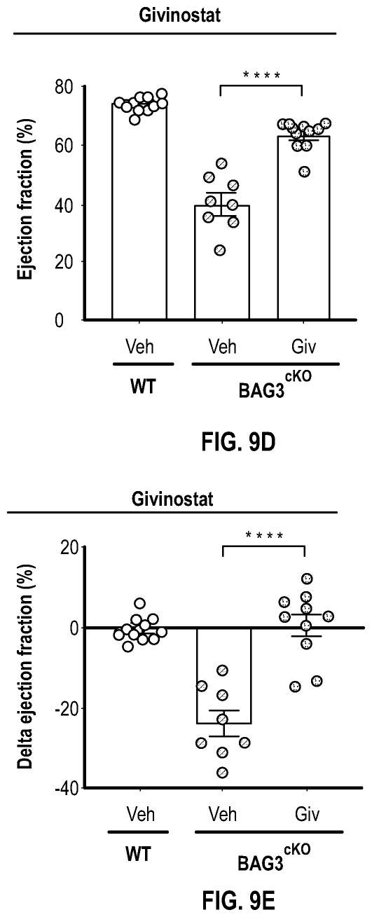

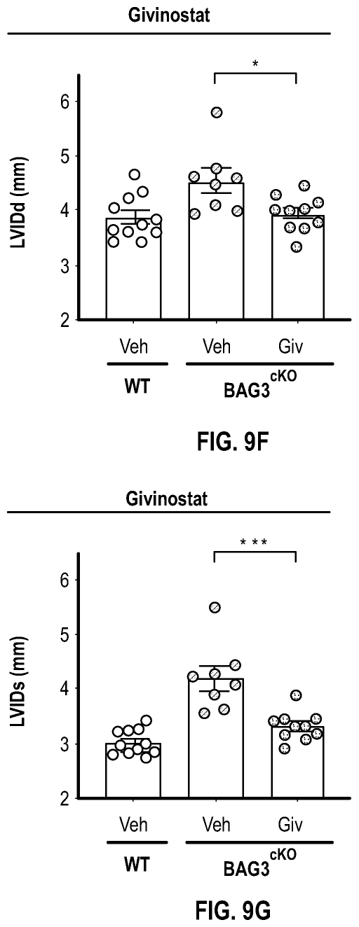

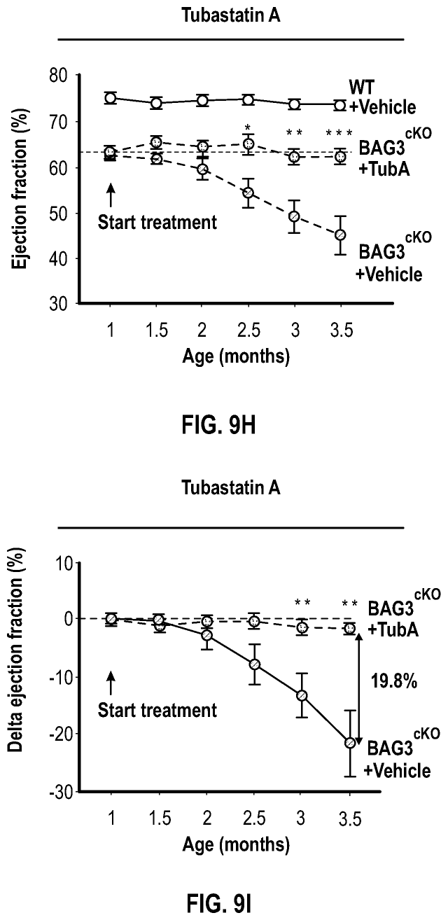

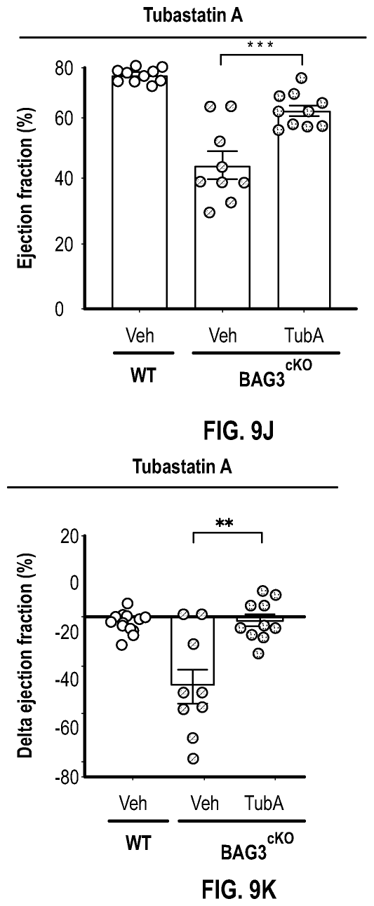

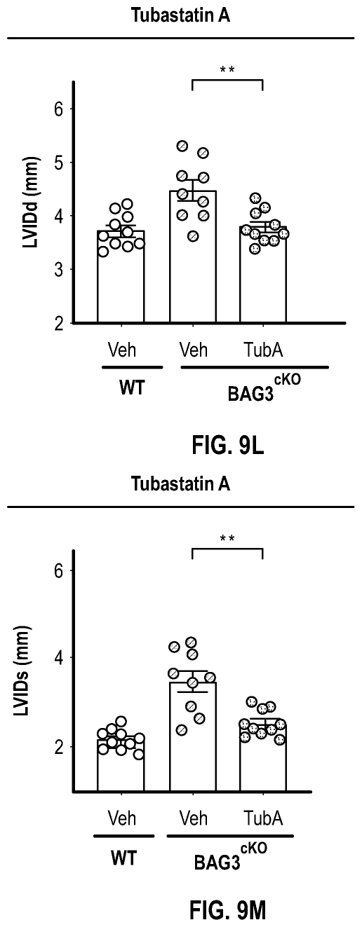

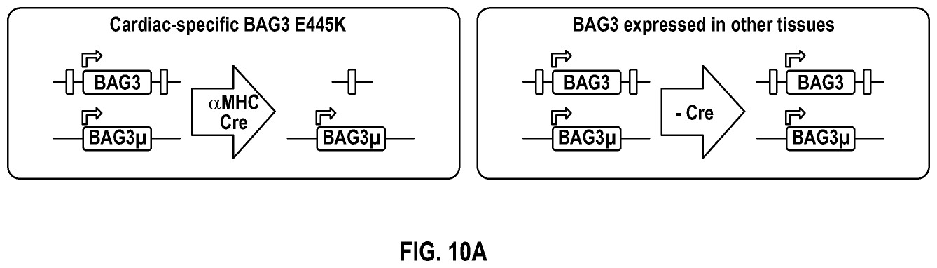

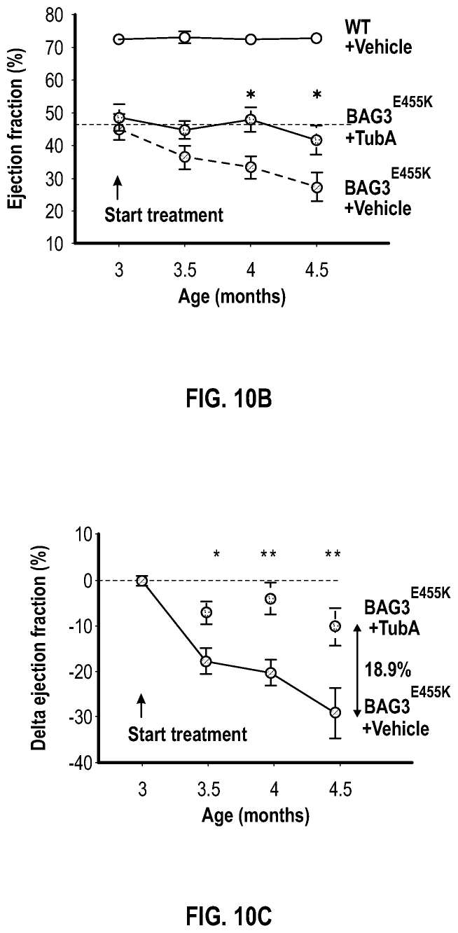

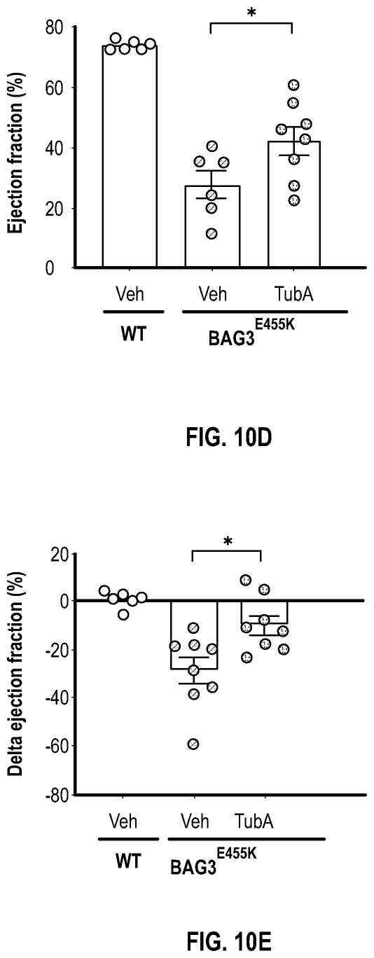

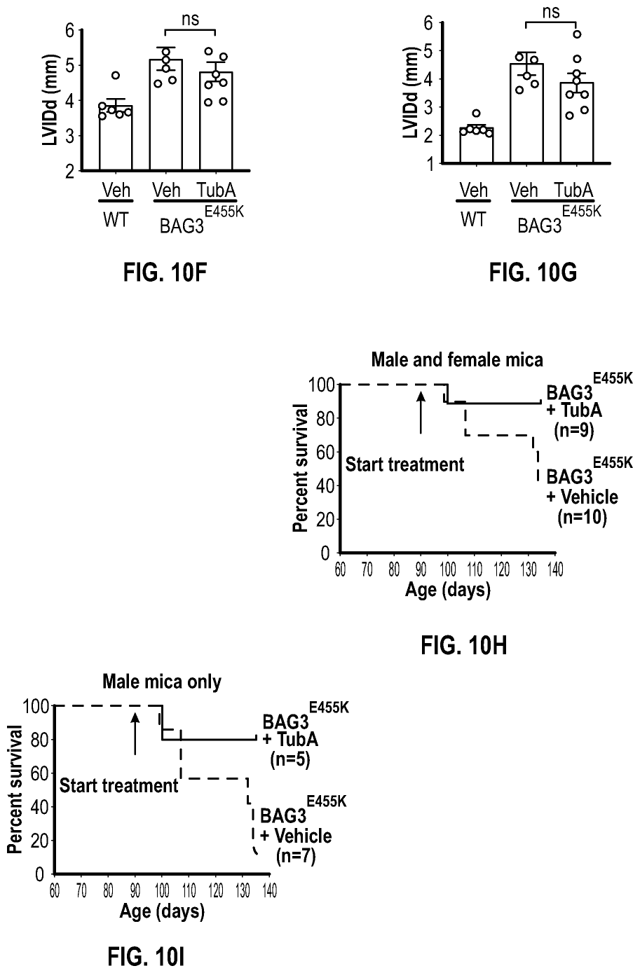

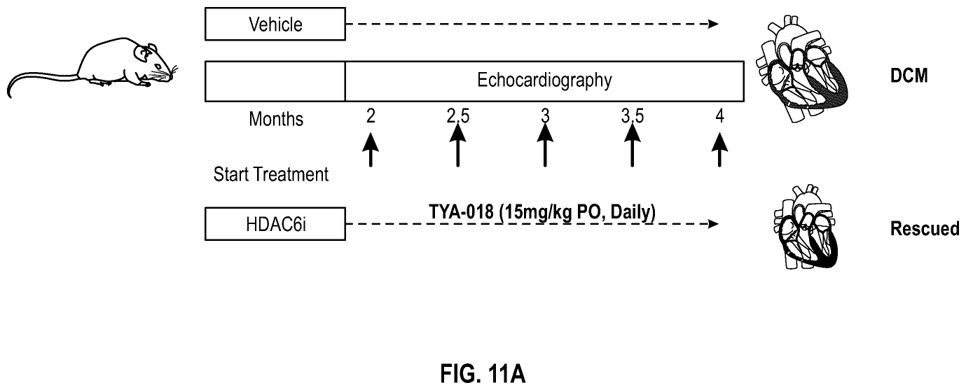

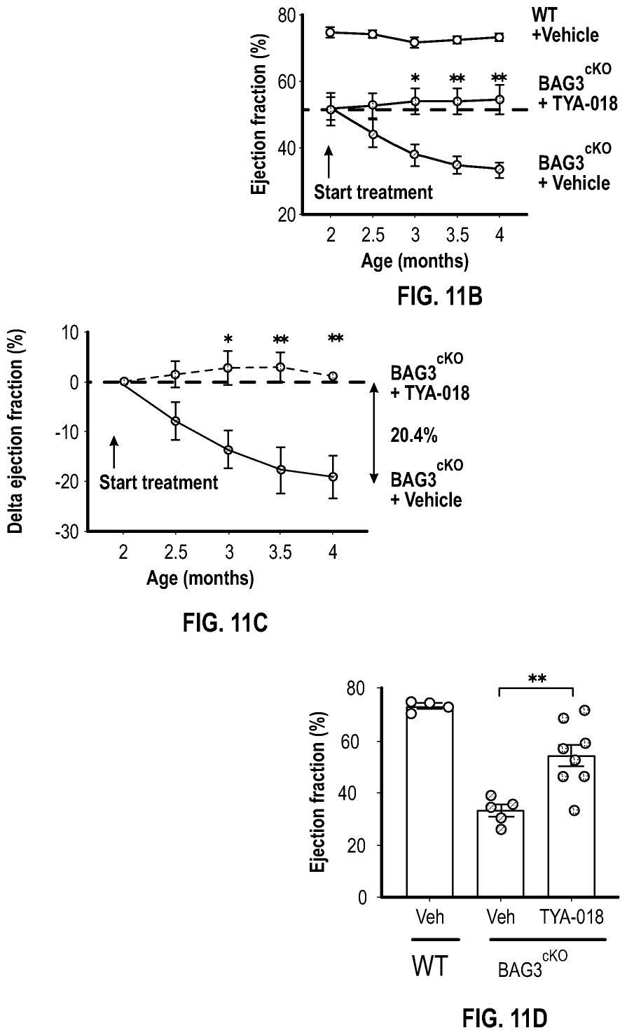

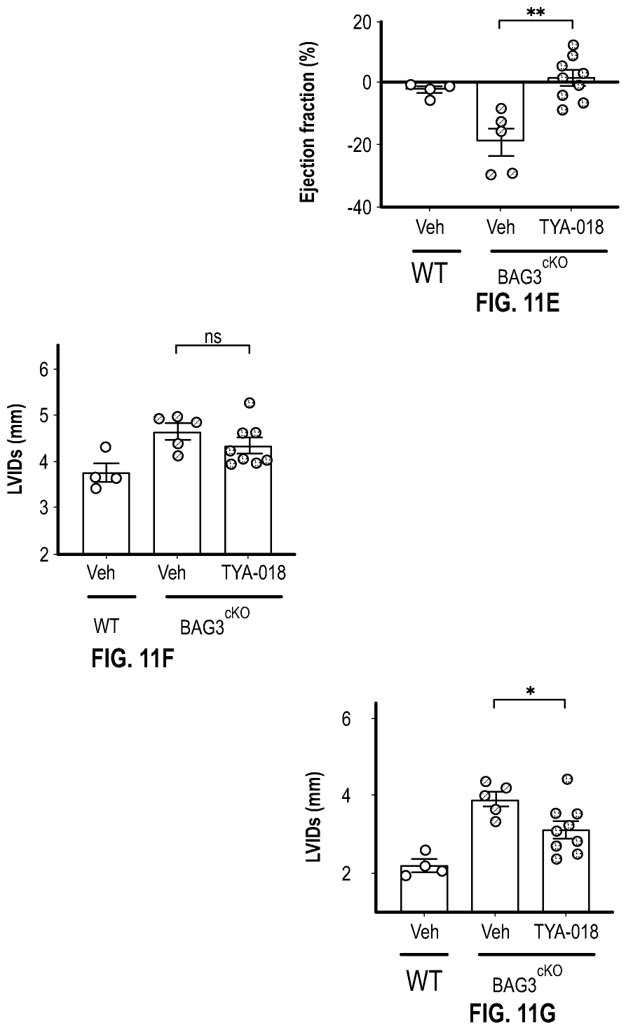

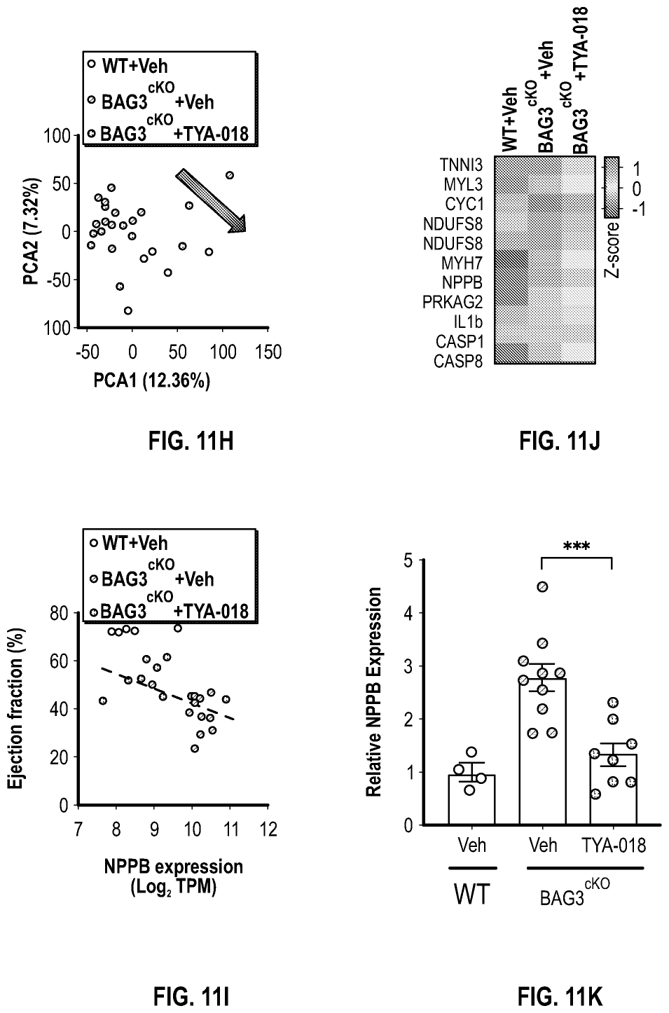

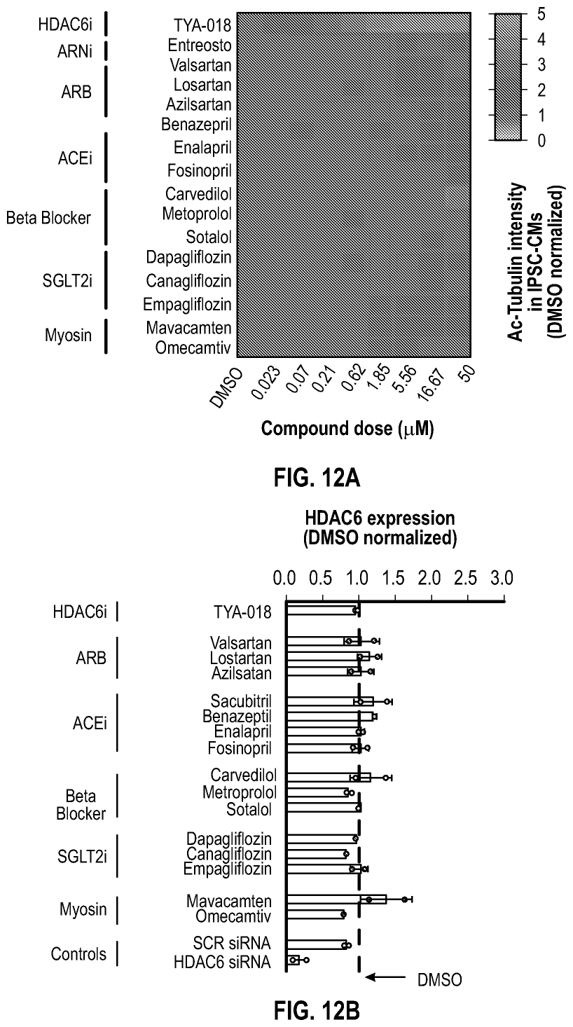

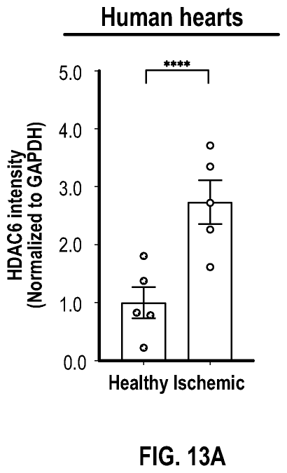

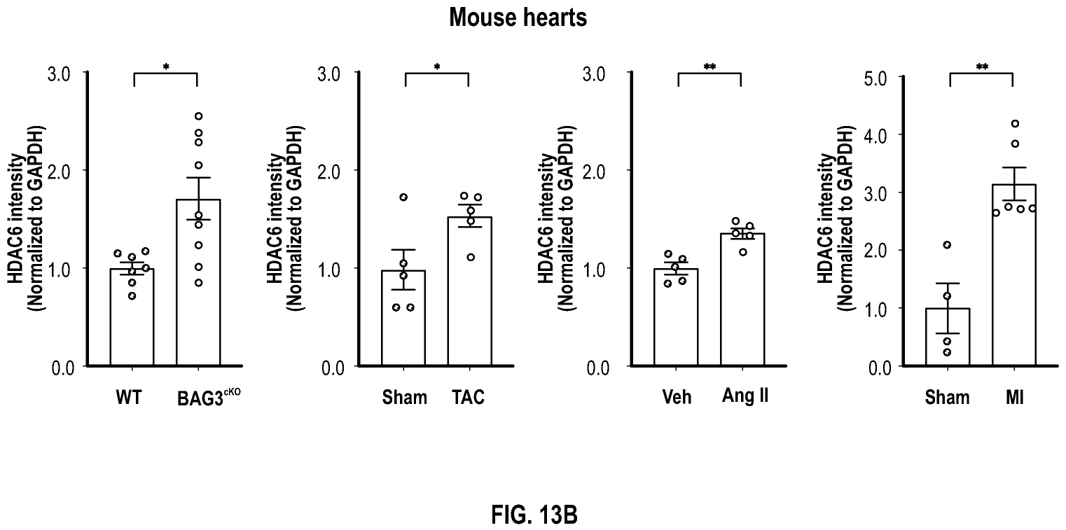

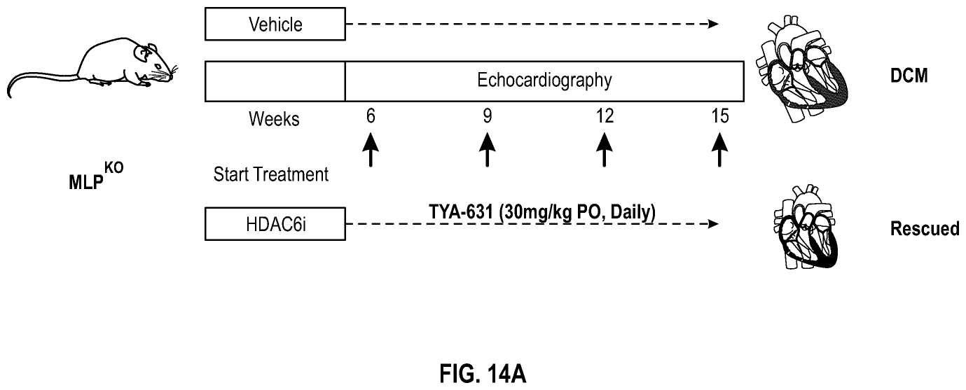

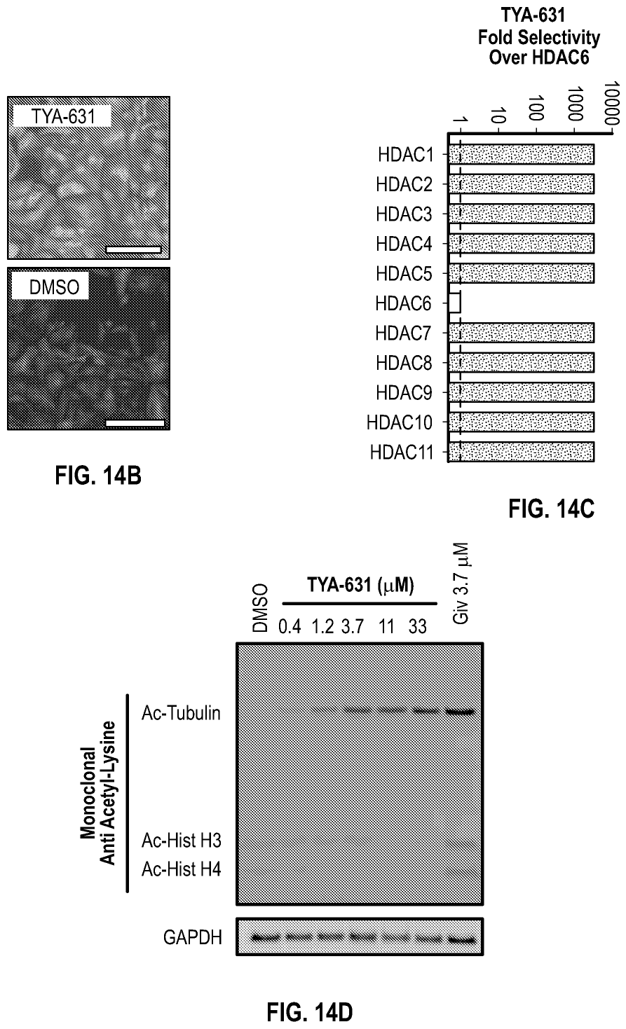

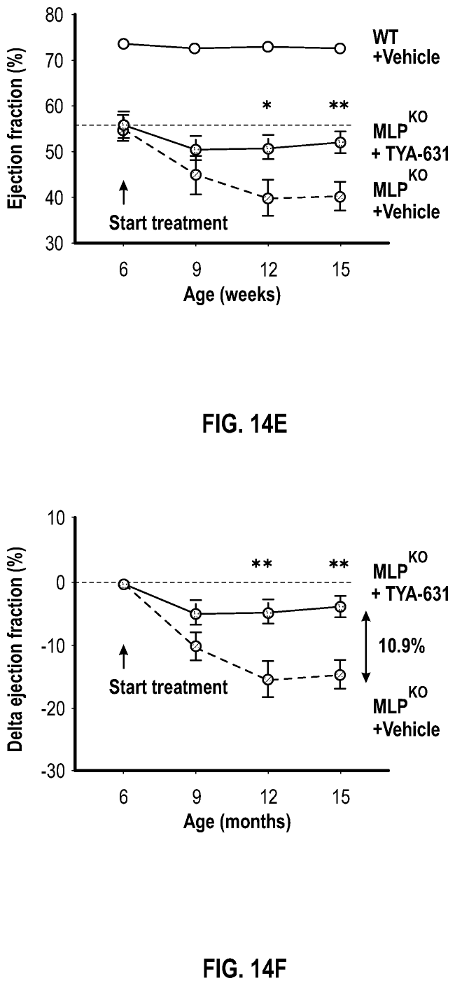

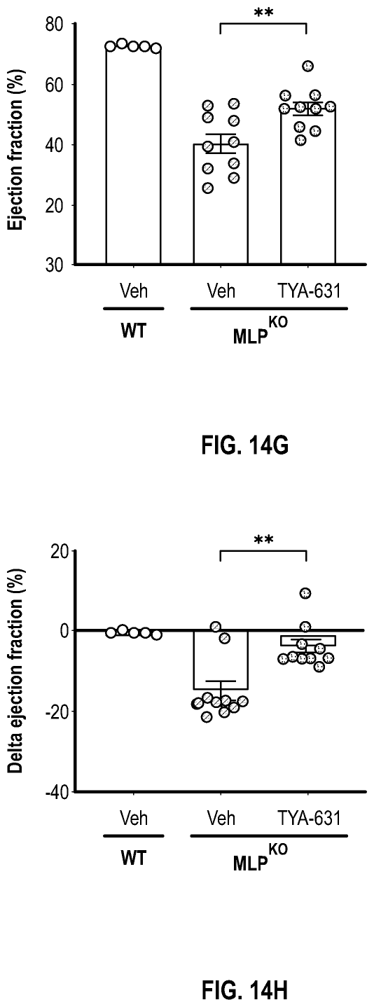

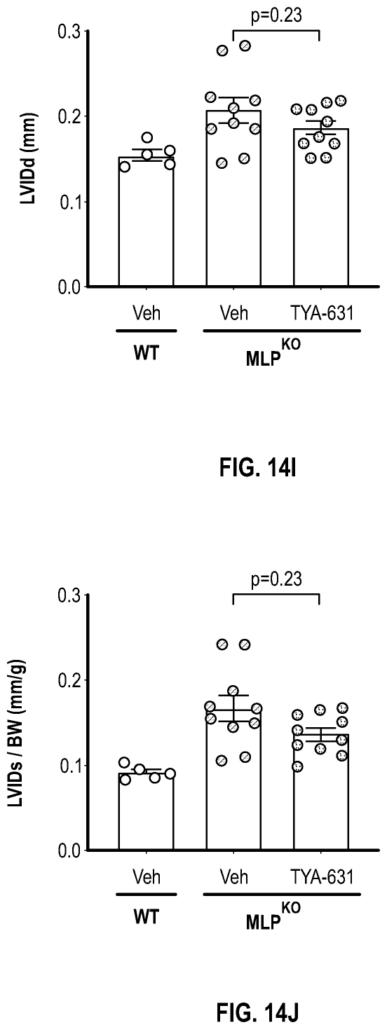

(FIG. 5A) Top compound classes (HDAC inhibitors, microtubule inhibitors) from the library screen and two cardiovascular standard-of-care agents [omecamtiv mecarbil (Omecamtiv) and sotalol] identified from the screen were validated at a 1 μM dose using the cardiomyocyte score. Data from 1-2 independent biological replicates. n=4−16 technical replicates per condition. Error bars=SD.

(FIG. 5B) Further validation using siRNAs showed that knockdown of HDAC6 protected against sarcomere damage in BAG3KD iPSC-CMs using the cardiomyocyte score with the deep learning algorithm. Data from 2-7 independent biological replicates. n=4−16 technical replicates per condition. Error bars=SD. ****P<0.0001.

(FIG. 5C) Representative immunostaining of anti-MYBPC3 in iPSC-CMs treated with scramble (SCR), BAG3, or BAG3+HDAC6 siRNA. Arrows indicate sarcomere damage. Scale bar=50 μm.

FIGS. 6A-6C show siRNA knockdown of HDAC6 is essential and sufficient to protect against cardiomyocyte damage induced by BAG3 loss-of-function

(FIG. 6A) Cardiomyocyte score of iPSC-CMS treated with four siRNAs targeting HDAC1 through HDAC11, individually and pooled (p), along with BAG3 siRNA. Two independent siRNAs targeting HDAC6 and as a pool showed an improved cardiomyocyte score. n=4−16 technical replicates per condition. Error bars=SD. ***P<0.001.

(FIG. 6B) Immunocytochemistry showing that BAG3 protein levels were ˜60% after knockdown. Co-knockdown of HDAC1 through HDAC11 with BAG3 did not increase BAG3 levels. n=5−16 technical replicates. Error bars=SD. ***P<0.001.

(FIG. 6C) Immunocytochemistry showing knockdown of HDAC3 and HDAC6 increased tubulin acetylation (Ac-Tubulin) in iPSC-CMs. n=5-16 technical replicates. Error bars=SD.

FIGS. 7A-7D show TYA-018 is a highly selective HDAC6 inhibitor.

(FIG. 7A) Biochemical assays using recombinant human HDAC6 and HDAC1 deacetylase activity show HDAC6 (on-target) and HDAC1 (off-target) inhibition curves following treatment with givinostat (pan-HDAC inhibitor), tubastatin A (HDAC6-selective inhibitor), and TYA-018 (HDAC6-selective inhibitor). Error bars=SD.

(FIG. 7B) Cell-based assay in iPSC-CMs shows dose-response curve of tubulin acetylation (Ac-Tubulin) as a function of drug concentration. Based on the calculated EC₅₀, givinostat, tubastatin A, and TYA-018 have similar ranges of cellular potencies for HDAC6 (ranging from 0.1 μM to 0.3 μM), with TYA-018 being the most potent. Error bars=SD. EC₅₀, half maximal effective concentration.

(FIG. 7C) Immunostaining of iPSC-CMs treated with 5.5 μM of each drug stained with anti-Ac-Tubulin antibody. Scale bar=200 μm.

(FIG. 7D) Western blot of iPSC-CMs treated with TYA-018 (HDAC6-specific inhibitor) stained with monoclonal anti-Ac-Lysine. Givinostat (Giv; pan-HDAC inhibitor control) showed both on-target (Ac-Tubulin stain) and off-target (Ac-Histone H3 and H4 stain) activity. TYA-018 only shows specific on-target activity with no detectable off-target activity, even at 33 μM.

FIGS. 8A-8C show TYA-018 is an exquisitely selective HDAC6 inhibitor as measured in biochemical and cell-based assays.

(FIG. 8A) Biochemical assay measuring deacetylase activity of HDAC1 through HDAC11 in the presence givinostat (pan-HDAC inhibitor), tubastatin A (HDAC6-selective inhibitor), and TYA-018 (HDAC6-selective inhibitor).

(FIG. 8B) Selectivity over HDAC6 activity showed TYA-018 has greater than 2500-fold selectivity for HDAC6 over other HDACs.

(FIG. 8C) Pro-BNP in iPSC-CMs after 4 days of incubation with drugs shows TYA-018 does not induce cellular stress. Error bars=SD.

FIGS. 9A-9M show givinostat and tubastatin A protect heart function in BAG3^{cKO} mouse

(FIG. 9A) Schematic of drug treatments in BAG3^{cKO} mouse model. Daily dosing began at 1 month of age. Givinostat (pan-HDAC inhibitor) was administered daily by oral gavage (PO) at 30 mg/kg. Tubastatin A (HDAC6-selective inhibitor) was administered daily by intraperitoneal injection (IP) at 50 mg/kg.

(FIG. 9B) Ejection fraction indicated that daily dosing of givinostat (Giv) protected heart function during the 10-week dosing period. Error bars=SEM. ***P<0.001, ****P<0.0001.

(FIG. 9C) Ejection fraction was tracked from the first day of dosing, and the delta ejection fraction was measured. During the 10-week period, heart function declined by 0.6% in the givinostat-treated arm (not significant), whereas it dropped by an average of 23.9% in the vehicle-treated arm (****P<0.0001). Error bars=SEM.

Ejection fraction (FIG. 9D) and delta ejection fraction (FIG. 9E) (compared to the pre-dose baseline) at 3.5 months of age and 10 weeks of dosing shows givinostat protects against declining heart function in BAG3^{cKO} mice. Error bars=SEM. ****P<0.0001.

Left ventricular internal diameter at diastole (LVIDd) (FIG. 9F) and systole (LVIDs) (FIG. 9G) were significantly reduced in BAG3^{cKO} mice treated with givinostat, bringing them closer to the levels seen in their WT littermates. Error bars=SEM. *P<0.05, ***P<0.001.

(FIG. 9H) Ejection fraction indicated that daily dosing of tubastatin A (TubA) protected heart function during the 10-week dosing period. Error bars=SEM. *P<0.05, **P<0.01, ***P<0.001.

(FIG. 9I) Ejection fraction was tracked from the first day of dosing, and delta ejection fraction was measured. During the 10-week period, heart function declined by 1.7% in BAG3^{cKO} mice treated with tubastatin A (not significant), whereas it dropped by 21.5% in mice treated with vehicle (**P<0.01). Error bars=SEM.

Ejection fraction (FIG. 9J) and delta ejection fraction (FIG. 9K) (compared to the pre-dose baseline) at 3.5 months of age and 10 weeks of dosing shows tubastatin A protected against declining heart function in BAG3^{cKO} mice. **P<0.01, ***P<0.001.

Tubastatin A significantly reduced LVIDd (FIG. 9L) and LVIDs (FIG. 9M) in BAG3^{cKO} mice to levels seen in their WT littermates. Error bars=SEM. **P<0.01.

FIGS. 10A-10I show BAG3^{E455K} mice develop heart failure and are protected by administration of tubastatin A.

(FIG. 10A) Schematic of the BAG3^{E455K} mouse model in which the WT BAG3 allele is floxed with LoxP sites and removed after αMHC-cre driven excision, leaving the E455K mutated form of BAG3 (BAG3p) expressed in the heart. BAG3 (WT and E445K) is expressed in other tissues.

(FIG. 10B) Treatment with tubastatin A (TubA; an HDAC6-selective inhibitor) began at 3 months of age. Daily dosing at 50 mg/kg significantly protected mice against declines in heart function compared to the vehicle-treated group. Error bars=SEM. *P<0.05.

(FIG. 10C) Ejection fraction was tracked from the first day of dosing, and delta ejection fraction was measured. Data show 10.1% decline in tubastatin A-treated mice during the 6-week period, whereas the vehicle-treated arm dropped by 29.0%. Error bars=SEM. *P<0.05, **P<0.01.

(FIGS. 10D & FIG. 10E) Ejection fraction and delta ejection fraction (compared to the pre-dose baseline) at 4.5 months of age and 6 weeks of dosing shows tubastatin A (TubA) protects against declining heart function in the BAG3^{cKO} mice. Error bars=SEM. *P<0.05.

Tubastatin A reduced left ventricular internal diameter at diastole (LVIDd) (FIG. 10F) and systole (LVIDs) (FIG. 10G) in BAG3^{E455K} mice.

Kaplan-Meier plots show tubastatin A reduced mortality in BAG3^{E445K} mice during the 6-week treatment (FIG. 10H). This effect was more pronounced in male mice (FIG. 10I).

FIGS. 11A-11K show inhibiting HDAC6 with TYA-018 protects heart function in BAG3^{cKO} mice.

(FIG. 11A) Schematic of drug treatment in BAG3^{cKO} mouse model. TYA-018 (highly selective HDAC6 inhibitor) was administered daily by oral gavage at 15 mg/kg starting when mice were 2 months of age.

(FIG. 11B) Daily dosing of TYA-018 protected heart function during the 8-week dosing period as measured by ejection faction. Error bars=SEM. *P<0.05, **P<0.01.

(FIG. 11C) Ejection fraction was tracked from the first day of dosing, and delta ejection fraction was measured. During the 8-week period, heart function did not decline in the TYA-018-treated arm, whereas it dropped by 19.1% in the vehicle-treated arm. Error bars=SEM. *P<0.05, **P<0.01.

Ejection fraction (FIG. 11D) and delta ejection fraction (FIG. 11E) (compared to the pre-dose baseline) at 4 months of age and 8 weeks of dosing shows TYA-018 protects against declining heart function in BAG3^{cKO} mice. Error bars=SEM. **P<0.01.

Left ventricular internal diameter at diastole (LVIDd) (FIG. 11F) and systole (LVIDs) (FIG. 11G) were reduced by TYA-018 in BAG3^{cKO} mice, bringing the levels closer to that of their WT littermates. Error bars=SEM. *P<0.05.

(FIG. 11H) Hearts from all three arms of the study were analyzed using RNA-Seq. Principal component analysis of all coding genes showed a global correction of BAG3^{cKO}+TYA-018 colding genes toward WT mice. Veh, vehicle.

(FIG. 11I) RNA-Seq analysis shows NPPB expression increased by approximately fourfold in BAG3^{cKO} mice compared to WT mice at 4 months of age. TYA-018 treatment reduced NPPB levels by twofold in BAG3^{cKO} mice. The level of NPPB in BAG3^{cKO}+TYA-018 mice was anticorrelated with heart function.

(FIG. 11J) Heatmap of RNA-Seq analysis from a selected number of genes. The data shows correction of key sarcomere genes (MYH7, TNNI3, and MYL3) and genes regulating mitochondrial function and metabolism (CYC1, NDUFS8, NDUFB8, PPKARG2) in BAG3^{cKO}+TYA-018 mice. Inflammatory (IL-1β, NLRP3) and apoptosis (CASP1, CAPS8) markers were also reduced.

(FIG. 11K) qPCR analysis shows ˜3-fold increase in NPPB expression levels in BAG3^{cKO} mouse hearts. In BAG3^{cKO} mice treated with TYA-018, NPPB expression levels are significantly reduced close to WT levels. Error bars=SEM. ***P<0.001.

FIGS. 12A-12B show cardiovascular standard-of-care drugs do not impact Ac-Tubulin and HDAC6 expression

(FIG. 12A) Ac-Tubulin levels were measured in iPSC-CMs treated with five classes of cardiovascular drugs used as standards of care (SOC) in the clinic. ARNi, angiotensin receptor neprilysin; ARB, angiotensin II receptor blocker; ACEi, angiotensin-converting enzyme inhibitor; SGLT2, sodium glucose co-transporter 2. Data shows no impact of SOC agents on Ac-Tubulin levels.

(FIG. 12B) SOC agents did not significantly impact HDAC6 expression in iPSC-CMs as measured using qPCR. N=2 biological replicates with 4 technical replicates in each experiment per condition. Error bars=SD.

FIG. 13A shows that HDAC6 levels were higher in ischemic human hearts vs healthy human hearts. Error bars=SEM. *P<0.05, **P<0.01, ****P<0.0001.

FIG. 13B shows increased levels of HDAC6 in BAG3^{cKO} mice and heart failure mouse models.

FIGS. 14A-14J show inhibiting HDAC6 with TYA-631 protects heart function in MLP^{KO} mice

(FIG. 14A) Schematic of drug treatment in MLP^{KO} mouse model. TYA-631 (selective HDAC6 inhibitor) was administered daily by oral gavage at 30 mg/kg starting when mice were 1.5 months of age.

(FIG. 14B) Immunostaining of iPSC-CMs treated with TYA-631 (5.5 μM) results in hyper-Ac-Tubulin. Scale bar=200 μm.

(FIG. 14C) Biochemical selectivity of TYA-631 shows 3500-fold selectivity for HDAC6 over other HDACs.

(FIG. 14D) Western blot of iPSC-CMs treated with TYA-631 stained with monoclonal anti-Ac-Lysine. Givinostat (Giv; pan-HDAC inhibitor control) showed both on-target (Ac-Tubulin stain) and off-target (Ac-Histone H3 and H4 stain) activity. TYA-631 only shows specific on-target activity with no detectable off-target activity.

(FIG. 14E) Daily dosing of TYA-631 protected heart function during the 9-week dosing period as measured by ejection faction. Error bars=SEM. *P<0.05, **P<0.01.

(FIG. 14F) Ejection fraction was tracked from the first day of dosing, and delta ejection fraction was measured. Mice treated with TYA-631 during the 9-week period show a 4.0% decline, whereas it dropped by 14.8% in the vehicle-treated arm. Error bars=SEM. *P<0.05, **P<0.01.

Ejection fraction (FIG. 14G) and delta ejection fraction (FIG. 14H) (compared to the pre-dose baseline) at 15 weeks of age and 9 weeks of dosing shows TYA-631 protects against declining heart function in MLP^{KO} mice. Error bars=SEM. **P<0.01.

Left ventricular internal diameter at diastole (LVIDd) (FIG. 14I) and systole (LVIDs) (FIG. 14J) were reduced by TYA-631 in MLP^{KO} mice. Error bars=SEM.

### DETAILED DESCRIPTION

### Overview

The present disclosure relates generally to the demonstration, both in vitro and in vivo, of the efficacy of various HDAC6 inhibitors in dilated cardiomyopathy (DCM). For example, as disclosed herein, both tubastatin A (>100-fold selectivity over other HDACs) and TYA-018 (>2500-fold selectivity over other HDACs) are efficacious in a BAG3^{cKO} and BAG3^{E455K} mouse models of DCM. In addition, as disclosed herein, TYA-631 is efficacious in a MLP^{KO} mouse model of DCM. Further, the potency of a large number of various HDAC6 inhibitors against HDAC6 is disclosed herein.

Accordingly, the disclosure provides support for use of HDAC6 inhibitors for the treatment of DCM.

In some embodiments, provided herein are methods of treating or preventing dilated cardiomyopathy in a subject in need thereof, comprising administering (e.g., orally) to a subject (e.g., a human) a HDAC6 inhibitor. In some embodiments, provided herein are methods of treating or preventing dilated cardiomyopathy associated with a decreased ejection fraction in a subject in need thereof, comprising administering (e.g., orally) to the subject (e.g., a human) a HDAC6 inhibitor.

Advantageously, administration of a selective HDAC6 inhibitor may be less toxic than a pan-HDAC inhibitor. With being bound by theory, an HDAC6 inhibitor may 1) directly act at the sarcomere level by protecting microtubules against mechanical damage, 2) improve myocyte compliance, and/or 3) promote autophagic flux and clearance of misfolded and damaged proteins. HDAC6 inhibition may directly stabilize and protect microtubules against damage and protect the Z-disk. Because sarcomere damage and myofibril disarray is a hallmark of DCM (Dominguez et al., 2018), inhibition of HDAC6 may provide protection at the sarcomere level in DCM.

### Definitions

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art.

Also as used herein, “and/or” refers to and encompasses any and all possible combinations of one or more of the associated listed items, as well as the lack of combinations when interpreted in the alternative (“or”).

The term “a” or “an” refers to one or more of that entity, i.e. can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, the term “HDAC6” refers to the enzyme that in humans is encoded by the HDAC6 gene.

As used herein, the term “HDAC6 inhibitor” refers to a compound that inhibits at least one enzymatic activity of HDAC6.

An HDAC6 inhibitor may be a “selective” HDAC6 inhibitor. The term “selective” as used herein refers to selectivity against other HDACs, known in the art as “isozymes.” In some embodiments, the selectivity ratio of HDAC6 over HDAC1 is from about 5 to about 30,0000, e.g., about 5, about 10, about 20, about 30, about 40, about 50, about 60, about 70, about 80, about 90, about 100, about 1000, about 2000, about 3000, about 4000, about 5000, about 6000, about 7000, about 8000, about 9000, about 10,000, about 15,000, about 20,000, about 25,000, or about 30,000, including all values and ranges therebetween.

For example, a HDAC6 inhibitor may be at least 100-fold selective against HDAC6 compared to all other isozymes of HDAC. In some cases, selectivity may be determined by reference of another HDAC inhibitor, such as a pan-HDAC inhibitor—that is an inhibitor that inhibits HDACs other than HDAC6 in addition to HDAC6. Givinostat is an example of a pan-HDAC6 inhibitor. In some embodiments, a selective HDAC6 inhibitor inhibits HDACs other than HDAC6 at least 100-fold less effectively than givinostat.

As used herein, the term “treating” refers to acting upon a disease, disorder, or condition with an agent to reduce or ameliorate harmful or any other undesired effects of the disease, disorder, condition and/or their symptoms.

As used herein, the term “preventing” refers to reducing the incidence or risk of developing, or delaying the development of, harmful or any other undesired effects of the disease, disorder, condition and/or symptoms

“Administration,” “administering” and the like, refer to administration to a subject by a medical professional or by self-administration by the subject, as well as to indirect administration, which may be the act of prescribing a composition of the invention. Typically, an effective amount is administered, which amount can be determined by one of skill in the art. Any method of administration may be used. Administration to a subject can be achieved by, for example, oral administration, in liquid or solid form, e.g. in capsule or tablet form; intravascular injection; intramyocardial delivery; or other suitable forms of administration.

As used herein, the term “effective amount” and the like refers to an amount that is sufficient to induce a desired physiologic outcome (e.g., increased cardiac function, decreased mortality, or decreased risk/incidence of hospitalization, increased exercise capacity, or reduced expression of one or more biomarkers associated with heart failure-such as BNP). An effective amount can be administered in one or more administrations, applications or dosages. Such delivery is dependent on a number of variables including the time period which the individual dosage unit is to be used, the bioavailability of the composition, the route of administration, etc. It is understood, however, that specific amounts of the compositions for any particular subject depends upon a variety of factors including the activity of the specific agent employed, the age, body weight, general health, sex, and diet of the subject, the time of administration, the rate of excretion, the composition combination, severity of the particular disease being treated and form of administration.

As used herein, the terms “subject” or “patient” refers to any animal, such as a domesticated animal, a zoo animal, or a human. The “subject” or “patient” can be a mammal like a dog, cat, horse, livestock, a zoo animal, or a human. The subject or patient can also be any domesticated animal such as a bird, a pet, or a farm animal. Specific examples of “subjects” and “patients” include, but are not limited to, individuals with a cardiac disease or disorder, and individuals with cardiac disorder-related characteristics or symptoms.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, see-propyl, n-butyl, i-butyl, see-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, and the like. The alkylene chain is attached to the rest of the molecule through a single bond and to a radical group (e.g., those described herein) through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to an unsaturated, straight or branched divalent hydrocarbon chain radical having one or more olefins and from two to twelve carbon atoms. Non-limiting examples of C₂-C₁₂ alkenylene include ethenylene, propenylene, n-butenylene, and the like. The alkenylene chain is attached to the rest of the molecule through a single bond and to a radical group (e.g., those described herein) through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to an unsaturated, straight or branched divalent hydrocarbon chain radical having one or more alkynes and from two to twelve carbon atoms. Non-limiting examples of C₂-C₁₂ alkynylene include ethynylene, propynylene, n-butynylene, and the like. The alkynylene chain is attached to the rest of the molecule through a single bond and to a radical group (e.g., those described herein) through a single bond. The points of attachment of the alkynylene chain to the rest of the molecule and to the radical group can be through any two carbons within the chain having a suitable valency. Unless stated otherwise specifically in the specification, an alkynylene chain can be optionally substituted.

“Alkoxy” refers to a group of the formula —OR^{a} where R^{a} is an alkyl, alkenyl or alkynl as defined above containing one to twelve carbon atoms. Unless stated otherwise specifically in the specification, an alkoxy group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

“Carbocyclyl,” “carbocyclic ring” or “carbocycle” refers to a rings structure, wherein the atoms which form the ring are each carbon, and which is attached to the rest of the molecule by a single bond. Carbocyclic rings can comprise from 3 to 20 carbon atoms in the ring. Carbocyclic rings include aryls and cycloalkyl, cycloalkenyl, and cycloalkynyl as defined herein. Unless stated otherwise specifically in the specification, a carbocyclyl group can be optionally substituted.

“Carbocyclylalkyl” refers to a radical of the formula —R_{b}—R_{d} where R^{b} is an alkylene, alkenylene, or alkynylene group as defined above and R_{d} is a carbocyclyl radical as defined above. Unless stated otherwise specifically in the specification, a carbocyclylalkyl group can be optionally substituted.

“Cycloalkyl” refers to a stable non-aromatic monocyclic or polycyclic fully saturated hydrocarbon consisting solely of carbon and hydrogen atoms, which can include fused or bridged ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyls include, for example, cyclopentenyl, cyclohexenyl, cycloheptenyl, cycloctenyl, and the like. Polycyclic cycloalkenyls include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl include, for example, cycloheptynyl, cyclooctynyl, and the like. Unless otherwise stated specifically in the specification, a cycloalkynyl group can be optionally substituted.

“Haloalkyl” refers to an alkyl, as defined above, that is substituted by one or more halo radicals, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Heterocyclyl,” “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclyl or heterocyclic rings include heteroaryls, heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused or bridged ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heteroaryl” refers to a 5- to 20-membered ring system comprising hydrogen atoms, one to nineteen carbon atoms, one to six heteroatoms selected from the group consisting of nitrogen, oxygen and sulfur, at least one aromatic ring, and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“Heterocyclylalkyl” refers to a radical of the formula —R_{b}—Rₑ where R^{b} is an alkylene, alkenylene, or alkynylene group as defined above and Rₑ is a heterocyclyl radical as defined above. Unless stated otherwise specifically in the specification, a heterocycloalkylalkyl group can be optionally substituted.

The term “substituted” used herein means any of the groups described herein (e.g., alkyl, alkenyl, alkynyl, alkoxy, aryl, aralkyl, carbocyclyl, cycloalkyl, cycloalkenyl, cycloalkynyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

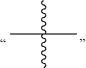

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

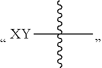

indicates that the chemical entity “XY” is bonded to another chemical entity via the point of attachment bond. Furthermore, the specific point of attachment to the non-depicted chemical entity can be specified by inference. For example, the compound CH₃-R³, wherein R³ is H or

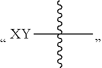

infers that when R³ is “XY”, the point of attachment bond is the same bond as the bond by which R³ is depicted as being bonded to CH₃.

As used herein, the term “restores” refers to increasing the level of biochemical or physiological parameter to a level observed in the subject prior to development of disease or condition, or to the level observed in a subject not having the disease or condition.

As used herein, the term “reduces” refers to decreasing the level of biochemical or physiological parameter.

As used herein, the term “cardiomyopathy” refers to any disease or dysfunction of the myocardium (heart muscle) in which the heart is abnormally enlarged, thickened and/or stiffened. As a result, the heart muscle's ability to pump blood is usually weakened. The etiology of the disease or disorder may be, for example, inflammatory, metabolic, toxic, infiltrative, fibroplastic, hematological, genetic, or unknown in origin. There are two general types of cardiomyopathies: ischemic (resulting from a lack of oxygen) and non-ischemic.

As used herein, the term “dilated cardiomyopathy” or “DCM” refers to condition in which the heart muscle becomes weakened and enlarged. As a result, the heart cannot pump enough blood to the rest of the body. The most common causes of dilated cardiomyopathy are heart disease caused by a narrowing or blockage in the coronary arteries; poorly controlled high blood pressure; alcohol or drug abuse; diabetes, thyroid disease, or hepatitis; drug side effects; abnormal heart rhythm; autoimmune illnesses; genetic causes; infection; heart valves that are either too narrow or too leaky; pregnancy; exposure to heavy metals such as lead, arsenic, cobalt, or mercury. DCM can affect anyone at any age. However, it is most common in adult men. DCM includes idiopathic DCM. In some embodiments, the DCM is familial DCM.

As used herein, the term “heart failure” refers to a condition in which the heart cannot pump enough blood to meet the body's need.

Heart failure is a complex clinical syndrome that can result from any structural or functional cardiovascular disorder causing systemic perfusion inadequate to meet the body's metabolic demands without excessively increasing left ventricular filling pressures. It is characterized by specific symptoms, such as dyspnea and fatigue, and signs, such as fluid retention.

As used herein, “chronic heart failure” or “congestive heart failure” or “CHF” refer, interchangeably, to an ongoing or persistent forms of heart failure. Common risk factors for CHF include old age, diabetes, high blood pressure and being overweight. CHF is broadly classified according to the systolic function of the left ventricle as HF with reduced or preserved ejection fraction (HFrEF and HFpEF). The term “heart failure” does not mean that the heart has stopped or is failing completely, but that it is weaker than is normal in a healthy person. In some cases, the condition can be mild, causing symptoms that may only be noticeable when exercising, in others, the condition may be more severe, causing symptoms that may be life-threatening, even while at rest. The most common symptoms of chronic heart failure include shortness of breath, tiredness, swelling of the legs and ankles, chest pain and a cough. In some embodiments, the methods of the disclosure decrease, prevent, or ameliorate one or more symptoms of heart failure in a subject suffering from or at risk for heart failure associated with DCM.

As used herein, the term “deleterious mutation” refers to a mutation that decreases the function of a gene. Deleterious mutations may include missense mutations, deletions or insertions in coding regions, non-coding mutations that influence gene expression or gene splicing, or others. Deleterious mutations include partial or total deletion of a gene. As used herein, the term may refer to homozygous or heterozygous mutations in a gene, provided the mutation manifests a phenotypic effect upon the carrier.

As used herein, the term “left ventricular internal diameter at diastole” or “LVIDd” refers to left ventricular size at diastole.

As used herein, the term “left ventricular internal diameter at systole” or “LVIDs” refers to left ventricular size at systole.

As used herein, the term “left ventricular mass” refers to the weight of the left ventricle.

As used herein, the term “ejection fraction” refers to the amount of blood being bumped out of the left ventricle each time it contracts, expressed as a percentage to the total amount of blood in left ventricle.

The detailed description of the disclosure is divided into various sections only for the reader's convenience and disclosure found in any section may be combined with that in another section. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

### Treating or Preventing Dilated Cardiomyopathy (DCM)

Provided are methods of treating or preventing dilated cardiomyopathy with an HDAC6 inhibitor.

### Familial DCM

In some embodiments, the DCM is familial DCM. Familial DCM has various known cases. Genes involved in familial DCM may include TTN, DSP, MYBPC3, SCN5A, RBM20, LDB3, LMNA, ANKRD1, MYH7, TNNT2, BAG3, DMD, MYPN, CSRP3 (also known as MLP), MYH6, TNNI3, ABCC9, TPM1, PSEN2, DES, or MYOZ2.

### BAG3

One gene that is essential to maintaining protein quality control is BCL2-associated athanogene 3 (BAG3). BAG3 is a stress-response gene, and it acts as an HSP70 co-chaperone in a complex with small heat shock proteins (HSPs) to maintain cardiomyocyte function (Franceschelli et al., 2008; Judge et al., 2017; Rauch et al., 2017). BAG3 is highly expressed in cardiac and skeletal muscle, and it can localize to the Z-disk (Homma et al., 2006). BAG3 has also been proposed to protect myocytes from mechanical damage and proteotoxic stress (Dominguez et al., 2018; Judge et al., 2017).

Mutations in BAG3 have been linked to DCM. In adults over 40 years old, loss-of-function BAG3 mutations show 80% penetrance of DCM (Dominguez et al., 2018). Familial BAG3 mutations are autosomal-dominant, suggesting a heterozygous loss-of-function mechanism (Chami et al., 2014; Judge et al., 2017; Villard et al., 2011). BAG3 mutations that result in loss-of-function account for approximately 3% of variant distribution in DCM genes (Haas et al., 2015). While most mutations in BAG3 are deleterious (e.g., E455K), a cardioprotective variant (C151R) has also been reported (Villard et al., 2011). This finding suggests that the BAG3 chaperone complex may acquire a gain-of-function phenotype that protects against proteotoxic stress and mechanical damage in the heart. In addition, mutations in BAG3 led to cardiac-related phenotypes in both in vivo and in vitro models, including zebrafish (Norton et al., 2011; Ruparelia et al., 2014), mice (Fang et al., 2017; Homma et al., 2006), and human induced pluripotent stem cell-derived cardiomyocytes (iPSC-CMs) (Judge et al., 2017). Also, appropriate BAG3 levels are required to maintain the chaperone function to maintain protein quality control, as reduced BAG3 was found in patients with idiopathic DCM (Feldman et al., 2014). Therefore, BAG3 is an attractive target for developing novel small-molecule therapeutics for BAG3 myopathies. These efforts could also lead to interventions for other genetic causes of DCM and non-genetic forms of heart failure (Stürner and Behl, 2017).

While most mutations in BAG3 are deleterious (e.g., E455K), a cardioprotective variant (C151R) has also been reported.

### Non-Familial DCM

In some embodiments, the DCM is non-familial DCM, including but not limited to idiopathic DCM. In some embodiments, the DCM is drug-induced cardiomyopathy (e.g., from anticancer or antiretroviral therapies), viral myocarditis, or postpartum cardiomyopathy.

### HDAC6 Inhibitors

Histone deacetylases (“HDAC”) are a class of enzymes with deacetylase activity with a broad range of genomic and non-genomic substrates. There are eleven Zinc-dependent HDAC enzymes classified based on sequence identity and catalytic activity (Haberland et al., 2009).

Histone deacetylase inhibitors have been described as a therapeutic agents in oncology (Yoon and Eom, 2016), neurodegeneration (Butler et al., 2010) autoimmune disease (Choi et al., 2018), chemotherapy-induced peripheral neuropathy (Krukowski et al., 2017) and cardiac indications (Zhang et al., 2002). Given the role of nuclear HDACs on regulating gene transcription, inhibition of these class of targets is known to have pleiotropic effects in various cell types; most notably resulting in cell toxicities. Therefore, limiting the toxicity of pan-HDAC inhibitors has been a major obstacle in wide-spread utilization for this class of compounds. In addition, significant adverse effects of pan-HDAC inhibitors (e.g. SAHA and Panobinostat) has been observed in the clinic including fatigue, nausea, diarrhea and thrombocytopenia (Subramanian et al., 2010).

In the cardiac-indication space, most studies have utilized pan-HDAC inhibitors (e.g. SAHA, TSA and Givinostat) for the treatment of pressure-overload rodent models including transverse aortic constriction (TAC) (Cao et al., 2011), hypertension in Dahl salt-sensitive rats (Jeong et al., 2018) and myocardial infarction (Nagata et al., 2019). In addition, HDAC6-selective inhibitors have been used to ameliorate the effects of pressure overload in rodent models (Demos-Davies et al., 2014) and provide protection against proteotoxicity in a transgenic cardiomyopathy mouse model (McLendon et al., 2014). However, these experiments in pressure overload rodent models are not predictive of treatment for dilated cardiomyopathy. Pressure overload in adult mice induces cardiomyocyte hypertrophy through an increase in cardiomyocyte cell size, enhanced protein synthesis, and new sarcomere assembly. Mohammadi et al. Nature Protocols 16:775-790 (2021). Pressure overload is a model of physiological, extrinsic damage to otherwise normal cardiomyocytes. Whereas dilated cardiomyopathy results from intrinsic defects into heart muscle cells (e.g., mutations in genes associated with cardiac function). Moreover, pressure overload models hypertrophic disease, not the heart muscle weakness of dilated cardiomyocyte.

HDAC6 belongs to the class IIb enzyme and contains two catalytic domains, a ubiquitin binding domain and a cytoplasmic retention domain (Haberland et al., 2009). HDAC6 is predominately a cytoplasmic enzyme and its best-characterized substrates include tubulin, HSP90 and cortactin (Brindisi et al., 2019).

Pharmacological inhibition of HDAC6 blocks its deacetylase activity, thus resulting in hyperacetylation of its substrates, most notably tubulin (Hubbert et al., 2002).

HDAC6-selective inhibitors are known to have reduced cytotoxicity due to the cytoplasmic nature of HDAC6 substrates and reduced effects on nuclear targets (including H3K9 and c-MYC) and on global transcription (Nebbioso et al., 2017).

Hydroxamic acids are zinc chelators and have been used extensively in the development of pan- and HDAC-selective inhibitors. However, most hydroxamic-acid based HDAC inhibitors either lack the desired selectivity or show poor bioavailability with a poor pharmacokinetic profile (Butler et al., 2010; Santo et al., 2012).

Various selective HDAC6 are known in the art. In addition, using known methods it is routine to screen compounds to identify further selective HDAC6 inhibitors. In particular, given a known HDAC6 inhibitor, a person of skill in the art can identify which analogs of the compound have selective HDAC6 activity.

In some embodiments, the HDAC6 inhibitor is a gene silencing agent, such as an RNA silencing agent (e.g., siRNA).

### Known HDAC6 Inhibitors

In some embodiments, the HDAC6 inhibitor is CAY10603, tubacin, rocilinostat (ACY-1215), citarinostat (ACY-241), ACY-738, QTX-125, CKD-506, nexturastat A, tubastatin A, or HPOB (listed in Table 1), or an analog thereof.

TABLE 1

| Compound | Description | IC₅₀ |
|---|---|---|
| CAY10603 | CAY10603 is a potent and selective HDAC6 inhibitor | 2 pM |
| | with IC50 of 2 pM, >200-fold selectivity over other HDACs. | |
| Tubacin | Tubacin is a highly potent and selective, reversible, cell- | 4 nM |
| | permeable HDAC6 inhibitor with an IC50 of 4 nM, approximately | |
| | 350-fold selectivity over HDAC1. | |
| Rocilinostat | Rocilinostat (ACY-1215) is a selective HDAC6 inhibitor | 4.7 nM |
| (ACY-1215) | with IC50 of 5 nM. It is >10-fold more selective for HDAC6 than | |
| | HDAC1/2/3 (class I HDACs) with slight activity against HDAC8, | |
| | minimal activity against HDAC4/5/7/9/11, Sirtuin1, and Sirtuin2. | |
| | Phase 2. | |
| Citarinostat | Citarinostat (ACY-241, HDAC-IN-2) is an orally available | 2.6 nM |
| (ACY-241) | selective HDAC6 inhibitor with IC50 of 2.6 nM and 46 nM for | |
| | HDAC6 and HDAC3, respectively. It has 13 to 18-fold selectivity | |
| | towards HDAC6 in comparison to HDAC1-3. | |
| ACY-738 | ACY-738 inhibits HDAC6 with low nanomolar potency (IC50 = 1.7 | 1.7 nM |
| | nM) and a selectivity of 60- to 1500-fold over class I HDACs. | |
| QTX-125 | HDAC6 inhibitor with 11-fold selectivity over HDAC1 | 0.6 nM |
| CKD-506 | HDAC6 inhibitor with an 80-fold selectivity over HDAC1 | 2.9 nM |
| Nexturastat A | Nexturastat A is a potent and selective HDAC6 inhibitor | 5 nM |
| | with IC50 of 5 nM, >190-fold selectivity over other HDACs. | |
| Tubastatin A | Tubastatin A is a potent and selective HDAC6 inhibitor | 15 nM |
| | with IC50 of 15 nM. It is selective against all the other isozymes | |
| | (1000-fold) except HDAC8 (57-fold). | |
| HPOB | HPOB is a potent, selective HDAC6 inhibitor with IC50 of 56 nM, | 56 nM |
| | >30-fold selectivity over other HDACs. | |

Further illustrative HDAC6 inhibitors are provided in U.S. patenttent Publications Nos. U.S. Pat. No. 8,227,516B2, US20100292169A1, US20070207950A1, U.S. Pat. No. 8,222,423B2, US20100093824A1, US20100216796A1, U.S. Pat. No. 8,673,911B2, U.S. Pat. No. 8,217,076B2, U.S. Pat. No. 8,440,716B2, US20110195432A1, U.S. Pat. No. 8,624,040B2, U.S. Pat. No. 9,096,518B2, U.S. Pat. No. 8,431,538B2, US20120258993A1, U.S. Pat. No. 8,546,588B2, U.S. Pat. No. 8,513,421B2, US20140031368A1, US20120015943A1, US20120015942A1, US20140243335A1, US20130225543A1, U.S. Pat. No. 8,471,026B2, U.S. Pat. No. 9,238,028B2, U.S. Pat. No. 8,765,773B2, US RE47009E1, US20140294856A1, U.S. Pat. No. 9,512,083B2, U.S. Pat. No. 9,670,193B2, U.S. Pat. No. 9,345,905B2, U.S. Pat. No. 9,409,858B2, U.S. Pat. No. 9,663,825B2, US20150119327A1, US20150250786A1, U.S. Ser. No. 10/041,046B2, U.S. Pat. No. 9,586,973B2, US20160069887A1, US20140357512A1, U.S. Pat. No. 9,751,832B2, US20160228434A1, US20150105358A1, U.S. Ser. No. 10/660,890B2, US20160271083A1, US20150176076A1, US20200405716A1, U.S. Pat. No. 9,890,136B2, U.S. Ser. No. 10/287,255B2, US20170173083A1, U.S. Ser. No. 10/016,421B2, U.S. Pat. No. 9,987,258B2, U.S. Ser. No. 10/568,854B2, U.S. Ser. No. 10/106,540B2, U.S. Ser. No. 10/266,489B2, U.S. Pat. No. 9,993,459B2, U.S. Ser. No. 10/183,934B2, U.S. Ser. No. 10/494,354B2, U.S. Ser. No. 10/494,353B2, U.S. Ser. No. 10/112,915B2, U.S. Ser. No. 10/377,726B2, U.S. Ser. No. 10/829,462B2, U.S. Ser. No. 10/829,461B2, US20210009539A1, US20210009538A1, U.S. Ser. No. 10/239,845B2, U.S. Ser. No. 10/472,337B2, U.S. Ser. No. 10/479,772B2, U.S. Ser. No. 10/464,911B2, U.S. Ser. No. 10/584,117B2, U.S. Ser. No. 10/538,498B2, U.S. Ser. No. 10/011,611B2, U.S. Ser. No. 10/494,355B2, U.S. Ser. No. 10/040,769B2, U.S. Ser. No. 10/858,323B2, U.S. Ser. No. 10/654,814B2, US20190209559A1, US20190185462A1, US20190192521A1, US20190321361A1, US20200046698A1, US20190262337A1, US20190282573A1, US20190282574A1, US20200071288A1, U.S. Ser. No. 10/745,389B2, U.S. Ser. No. 10/357,493B2, US20200171028A1, US20200054773A1, US20200308174A1, US20200155549A1, U.S. Ser. No. 10/435,399B2, US20200216563A1, US20190216751A1, US20200339569A1, US20210078963A1, US20210077487A1, US20190270733A1, US20190270744A1, US20200022966A1, and US20210094944A1, which are incorporated herein for purposes of identifying HDAC6 inhibitors that may be used in the methods disclosed herein. In some embodiments, the HDAC6 inhibitor is TYA-631 or an analog thereof.

### Fluoroalkyl-Oxadiazole Derivatives

In some embodiments, the HDAC6 inhibitor is a fluoroalkyl-oxadiazole derivative. Illustrative fluoroalkyl-oxadiazole derivatives that may be used as HDAC6 inhibitors include those described herein and those described in Int'l Pat. Appl. No. PCT/US2020/066439, published as WO2021127643A1 the content of which is incorporated by reference herein in its entirety. PCT/US2020/066439, published as WO2021127643A1, also describes methods of synthesis of such compounds, which are specifically incorporated by reference herein.

In some embodiments, the HDAC6 inhibitor is a compound of Formula (I):

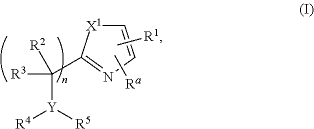

wherein
R¹ is selected from the group consisting of

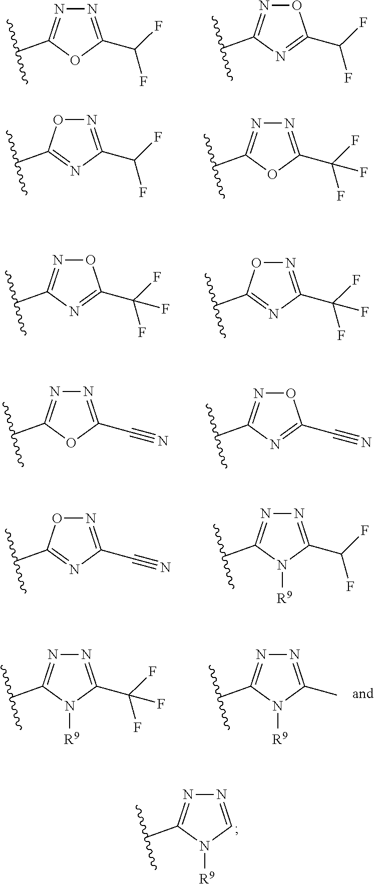

R^{a} is selected from the group consisting of H, halo, C₁₋₃ alkyl, cycloalkyl, haloalkyl, and alkoxy;
R² and R³ are independently selected from the group consisting of H, halogen, alkoxy, haloalkyl, aryl, heteroaryl, alkyl, and cycloalkyl each of which is optionally substituted, or R² and R³ together with the atom to which they are attached form a cycloalkyl or heterocyclyl;
R⁴ and R⁵ are independently selected from the group consisting of H, —(SO₂)R², —(SO₂)NR²R³, —(CO)R², —(CONR²R³), aryl, arylheteroaryl, alkylenearyl, heteroaryl, cycloalkyl, heterocyclyl, alkyl, haloalkyl, and alkoxy, each of which is optionally substituted, or R⁴ and R⁵ together with the atom to which they are attached form a cycloalkyl or heterocyclyl, each of which is optionally substituted;
R⁹ is selected from the group consisting of H, C₁-C₆ alkyl, haloalkyl, cycloalkyl and heterocyclyl;
X¹ is selected from the group consisting of S, O, NH and NR⁶, wherein R⁶ is selected from the group consisting of C₁-C₆ alkyl, alkoxy, haloalkyl, cycloalkyl and heterocyclyl;
Y is selected from the group consisting of CR², O, N, S, SO, and SO₂, wherein when Y is O, S, SO, or SO₂, R⁵ is not present and when R⁴ and R⁵ together with the atom to which they are attached form a cycloalkyl or heterocyclyl, Y is CR² or N; and
n is selected from 0, 1, and 2.

In some embodiments of Formula (I), n is 0. In some embodiments, n is 1. In some embodiments n is 2. In some embodiments, n is 0 or 1. In some embodiments n is 1 or 2. In some embodiments n is 0 or 2.

In some embodiments of Formula (I), X¹ is O. In some embodiments, X¹ is S. In some embodiments, X¹ is NH. In some embodiments, X¹ is NR⁶. In some embodiments, X¹ is selected from the group consisting of S, O, and NR⁶. In some embodiments, X¹ is selected from the group consisting of S, O, and NCH₃. In some embodiments, X¹ is S or O. In some embodiments, X¹ is S or NR⁶. In some embodiments, R⁶ is C₁-C₆ alkyl.

In some embodiments of Formula (I), R² and R³ are H.

In some embodiments of Formula (I), Y is N, CR², or O. In some embodiments, Y is N or O. In some embodiments, Y is N. In some embodiments, Y is CR². In some embodiments, Y is O.

In some embodiments, R⁴ and R⁵ are independently selected from the group consisting of H, —(SO₂)R², —(SO₂)NR²R³, —(CO)R², —(CONR²R³), aryl, arylheteroaryl, heteroaryl, alkylenearyl, cycloalkyl, alkylenecycloalkyl, heterocyclyl, alkyleneheterocyclyl, alkyl, haloalkyl, and alkoxy, each of which is optionally substituted, or R⁴ and R⁵ together with the atom to which they are attached form a cycloalkyl or heterocyclyl, each of which is optionally substituted

In some embodiments of Formula (I), R⁴ is selected from the group consisting of —C(O)— alkyl, —C(O)-cycloalkyl, —C(O)-aryl, —C(O)-heteroaryl, —(SO₂)NR²R³, —SO₂-alkyl, and —SO₂-cycloalkyl, each of which is optionally substituted. In some embodiments, R⁴ is selected from the group consisting of —C(O)-alkyl, —C(O)-cycloalkyl, —SO₂-alkyl, —SO₂-haloalkyl, —SO₂-cycloalkyl, and —(SO₂)NR²R³, each of which is optionally substituted. In some embodiments, aryl is optionally substituted with one or more halogens. In some embodiments of Formula (I), R⁴ is selected from the group consisting of —SO₂ alkyl, —SO₂ haloalkyl, or —SO₂ cycloalkyl. In some embodiments of Formula (I), R⁴ is selected from the group consisting of —SO₂Me, —SO₂Et, and —SO₂-cPr. In some embodiments, R² and R³ are each independently —C₁₋₅ alkyl. In some embodiments, R² and R³ taken together with the nitrogen atom to which they are attached form an optionally substituted heterocyclyl. In some embodiments, the optionally substituted heterocyclyl is morpholine, thiomorpholine, or thiomorpholine 1,1-dioxide.

In some embodiments of Formula (I), R⁵ is aryl, heteroaryl, or cycloalkyl, each of which is optionally substituted.

In some embodiments, R⁵ is aryl. In some embodiments, aryl is

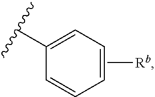

wherein R^{b} is one or more selected from the group consisting of halogen, haloalkyl, alkyl, Oalkyl, Ohaloalkyl, alkylene-Ohaloalkyl, cycloalkyl, heterocyclyl aryl, heteroaryl, alkylnitrile, or CN. In some embodiments, the haloalkyl is selected from CF₃, CF₂CH₃, CHF₂, or CH₂F. In some embodiments, the alkyl is a —C₁₋₅ alkyl. In some embodiments, —C₁₋₅ alkyl is methyl, ethyl, propyl, i-propyl, butyl, or t-butyl. In some embodiments, methyl, ethyl, propyl, i-propyl, butyl, or t-butyl is optionally substituted with OH. In some embodiments, the cycloalkyl is a C₃₋₆ cycloalkyl. In some embodiments, the aryl is a phenyl. In some embodiments, the heteroaryl is 5- or 6-membered heteroaryl having 1, 2, or 3 heteroatoms selected from N, O, and S. In some embodiments, the heterocyclyl is a 4- to 7-member heterocyclyl with 1 or 2 heteroatoms selected from N, O, and S. In some embodiments, the Ohaloalkyl is selected from OCF₃, OCHF₂, or OCH₂F. In some embodiments, the Oalkyl is O-methyl, O-ethyl, O-propyl, O-i-propyl, O-butyl, or O-t-butyl.

In some embodiments, R⁵ is heteroaryl. In some embodiments, heteroaryl is an optionally substituted 5- to 14-membered heteroaryl. In some embodiments, heteroaryl is an optionally substituted 5- to 14-membered heteroaryl having 1, 2, or 3 heteroatoms selected from the group consisting of N, O, and S. In some embodiments, the optionally substituted 5- to 14-membered heteroaryl is selected from the group consisting of pyrazolyl, imidazolyl, oxazolyl, thiazolyl, pyridinyl, pyrimidinyl, pyrazinyl, pyridazinyl, quinolinyl, isoquinolinyl, quinoxalinyl, cinnolinyl, indolizinyl, azaindolizinyl, indolyl, azaindolyl, benzoxazolyl, benzthiazolyl, benzfuranyl, benzthiophenyl, imidazopyridinyl, imidazopyrazinyl, and benzimidazolyl. In some embodiments, the optionally substituted 5- to 14-membered heteroaryl is selected from the group consisting of pyrazolyl, pyridinyl, pyrimidinyl, pyrazinyl, pyridazinyl, benzoxazolyl, imidazopyridinyl, and imidazopyrazinyl. In some embodiments, R⁵ is

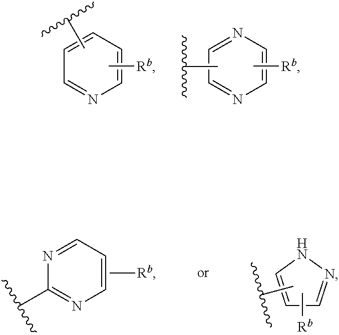

wherein R^{b} is one or more selected from the group consisting of halogen, haloalkyl, alkyl, Oalkyl, Ohaloalkyl, alkylene-Ohaloalkyl, cycloalkyl, heterocyclyl aryl, heteroaryl, alkylnitrile, or CN. In some embodiments, the haloalkyl is selected from CF₃, CF₂CH₃, CHF₂, or CH₂F. In some embodiments, the alkyl is a —C₁₋₅ alkyl. In some embodiments, —C₁₋₅ alkyl is methyl, ethyl, propyl, i-propyl, butyl, or t-butyl. In some embodiments, methyl, ethyl, propyl, i-propyl, butyl, or t-butyl is optionally substituted with OH. In some embodiments, the cycloalkyl is a C₃₋₆ cycloalkyl. In some embodiments, the aryl is a phenyl. In some embodiments, the heteroaryl is 5- or 6-membered heteroaryl having 1, 2, or 3 heteroatoms selected from N, O, and S. In some embodiments, the heterocyclyl is a 4- to 7-member heterocyclyl with 1 or 2 heteroatoms selected from N, O, and S. In some embodiments, the Ohaloalkyl is selected from OCF₃, OCHF₂, or OCH₂F. In some embodiments, the Oalkyl is O-methyl, O-ethyl, O-propyl, O-i-propyl, O-butyl, or O-t-butyl.

In some embodiments, R⁵ is cycloalkyl. In some embodiments, cycloalkyl is a cyclopropyl, cyclobutyl, cyclopentyl, or cyclohexyl, each of which is optionally substituted.

In some embodiments, the optionally substituted cycloalkyl is

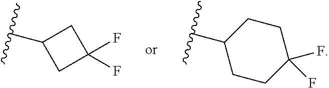 [SMILES: *C1CC(F)(F)C1 ; "or" ; *C1CCC(F)(F)CC1]

In some embodiments, R⁵ is selected from the group consisting of phenyl, 3-chlorophenyl, 3-chloro-4-fluorophenyl, 3-trifluoromethylphenyl, 3,4-difluorophenyl, and 2,6-difluorophenyl. In some embodiments, R⁵ is cyclopropyl. In some embodiments, R⁵ selected from the group consisting of pyridin-3-yl and 1-methylindazole-6-yl. In some embodiments, R⁵ is selected from the group consisting of H, phenyl, 3-chlorophenyl, 3-chloro-4-fluorophenyl, 3-trifluoromethylphenyl, 3,4-difluorophenyl, cyclopropyl, pyridin-3-yl, 1-methylindazole-6-yl, 3,3-difluorocyclobutyl, and 4,4-difluorocyclohexyl. In some embodiments, R⁵ is 3-chlorophenyl. In some embodiments R⁵ is H. In some embodiments, R⁵ is

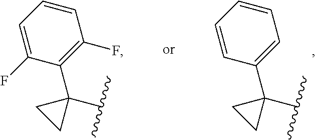 [SMILES: *C1(c2c(F)cccc2F)CC1 ; "or" ; *C1(c2ccccc2)CC1 ; ","]

In some embodiments, R⁵ is —CH₂CH₂Ph. In some embodiments, R⁵ is selected from the group consisting of H, aryl, heteroaryl, alkylenearyl, cycloalkyl, heterocyclyl, alkyl, and haloalkyl, each of which is optionally substituted, or R⁴ and R⁵ together with the atom to which they are attached form an optionally substituted heterocyclyl.

In some embodiments of Formula (I), R⁵ is optionally substituted with one or more halogen, haloalkyl, alkyl, Oalkyl, Ohaloalkyl, cycloalkyl, heterocyclyl aryl, or heteroaryl. In some embodiments, the haloalkyl is selected from CF₃, CHF₂, or CH₂F. In some embodiments, the alkyl is a —C₁₋₅ alkyl. In some embodiments, —C₁₋₅ alkyl is methyl, ethyl, propyl, i-propyl, butyl, or t-butyl. In some embodiments, the cycloalkyl is a C₃₋₆ cycloalkyl. In some embodiments, the aryl is a phenyl. In some embodiments, the heteroaryl is 5- or 6-membered heteroaryl having 1, 2, or 3 heteroatoms selected from N, O, and S. In some embodiments, the heterocyclyl is a 4- to 7-member heterocyclyl with 1 or 2 heteroatoms selected from N, O, and S. In some embodiments, the Ohaloalkyl is selected from OCF₃, OCHF₂, or OCH₂F. In some embodiments, the Oalkyl is O-methyl, O-ethyl, O-propyl, O-i-propyl, O-butyl, or O-t-butyl.

In some embodiments of Formula (I), R⁴ is H or —C₁₋₅ alkyl and R⁵ is aryl. In some embodiments, R⁴ is H or —C₁₋₅ alkyl and R⁵ is heteroaryl. In some embodiments, R⁴ is H or —C₁₋₅ alkyl and R⁵ is cycloalkyl. In some embodiments, the —C₁₋₅ alkyl is methyl, ethyl, or propyl. In some embodiments, the —C₁₋₅ alkyl is methyl. In some embodiments, the aryl is optionally substituted phenyl. In some embodiments, the heteroaryl is a 5- to 14-membered heteroaryl having 1, 2, or 3 heteroatoms selected from the group consisting of N, O, and S. In some embodiments, the optionally substituted 5- to 14-membered heteroaryl is selected from the group consisting of pyrazolyl, imidazolyl, oxazolyl, thiazolyl, pyridinyl, pyrimidinyl, pyrazinyl, pyridazinyl, quinolinyl, isoquinolinyl, quinoxalinyl, cinnolinyl, indolizinyl, azaindolizinyl, indolyl, azaindolyl, benzoxazolyl, benzthiazolyl, benzfuranyl, benzthiophenyl, imidazopyridinyl, imidazopyrazinyl, and benzimidazolyl. In some embodiments, the heteroaryl is a 5- or 6-membered heteroaryl ring. In some embodiments, the 5-membered heteroaryl is optionally substituted pyrazolyl, imidazolyl, or oxazolyl. In some embodiments, the 6-membered heteroaryl is optionally substituted pyridinyl, pyrimidinyl, pyrazinyl, or pyridazinyl. In some embodiments, cycloalkyl is optionally substituted cyclopropyl, cyclobutyl, cyclopentyl, or cyclohexyl. In some embodiments, aryl is optionally substituted with one or more substituents selected from the group consisting of halogen, C₁₋₆ haloalkyl, C₁₋₆ alkyl, O—C₁₋₆ alkyl, O—C₁₋₆ haloalkyl, or C₃₋₆ cycloalkyl. In some embodiments, heteroaryl is optionally substituted with one or more substituents selected from the group consisting of halogen, C₁₋₆ haloalkyl, C₁₋₆ alkyl, O—C₁₋₆ alkyl, O—C₁₋₆ haloalkyl, or C₃₋₆ cycloalkyl.

In some embodiments of Formula (I), R⁴ is —(CO)R² and R⁵ is aryl. In some embodiments, R⁴ is —(CO)R² and R⁵ is heteroaryl. In some embodiments, R⁴ is —(CO)R² and R⁵ is cycloalkyl. In some embodiments, the aryl is optionally substituted phenyl. In some embodiments, the aryl is optionally substituted phenyl. In some embodiments, the heteroaryl is a 5- to 14-membered heteroaryl having 1, 2, or 3 heteroatoms selected from the group consisting of N, O, and S. In some embodiments, the optionally substituted 5- to 14-membered heteroaryl is selected from the group consisting of pyrazolyl, imidazolyl, oxazolyl, thiazolyl, pyridinyl, pyrimidinyl, pyrazinyl, pyridazinyl, quinolinyl, isoquinolinyl, quinoxalinyl, cinnolinyl, indolizinyl, azaindolizinyl, indolyl, azaindolyl, benzoxazolyl, benzthiazolyl, benzfuranyl, benzthiophenyl, imidazopyridinyl, imidazopyrazinyl, and benzimidazolyl. In some embodiments, the heteroaryl is a 5- or 6-membered heteroaryl ring. In some embodiments, the 5-membered heteroaryl is optionally substituted pyrazolyl, imidazolyl, oxazolyl, In some embodiments, the 6-membered heteroaryl is optionally substituted pyridinyl, pyrimidinyl, pyrazinyl, or pyridazinyl. In some embodiments, cycloalkyl is optionally substituted cyclopropyl, cyclobutyl, cyclopentyl, or cyclohexyl. In some embodiments, aryl is optionally substituted with one or more substituents selected from the group consisting of halogen, C₁₋₆ haloalkyl, C₁₋₅ alkyl, O—C₁₋₆ alkyl, O—C₁₋₆ haloalkyl, or C₃₋₆ cycloalkyl. In some embodiments, heteroaryl is optionally substituted with one or more substituents selected from the group consisting of halogen, C₁₋₆ haloalkyl, C₁₋₆ alkyl, O—C₁₋₆ alkyl, O—C₁₋₆ haloalkyl, or C₃₋₆ cycloalkyl.

In some embodiments of Formula (I), R⁴ is —(SO₂)R² and R⁵ is aryl. In some embodiments, R⁴ is —(SO₂)R² and R⁵ is heteroaryl. In some embodiments, R⁴ is —(SO₂)R² and R⁵ is cycloalkyl. In some embodiments, the aryl is optionally substituted phenyl. In some embodiments, the heteroaryl is a 5- to 14-membered heteroaryl having 1, 2, or 3 heteroatoms selected from the group consisting of N, O, and S. In some embodiments, the optionally substituted 5- to 14-membered heteroaryl is selected from the group consisting of pyrazolyl, imidazolyl, oxazolyl, thiazolyl, pyridinyl, pyrimidinyl, pyrazinyl, pyridazinyl, quinolinyl, isoquinolinyl, quinoxalinyl, cinnolinyl, indolizinyl, azaindolizinyl, indolyl, azaindolyl, benzoxazolyl, benzthiazolyl, benzfuranyl, benzthiophenyl, imidazopyridinyl, imidazopyrazinyl, and benzimidazolyl. In some embodiments, the heteroaryl is a 5- or 6-membered heteroaryl ring. In some embodiments, the 5-membered heteroaryl is optionally substituted pyrazolyl, imidazolyl, or oxazolyl. In some embodiments, the 6-membered heteroaryl is optionally substituted pyridinyl, pyrimidinyl, pyrazinyl, or pyridazinyl. In some embodiments, cycloalkyl is optionally substituted cyclopropyl, cyclobutyl, cyclopentyl, or cyclohexyl. In some embodiments, aryl is optionally substituted with one or more substituents selected from the group consisting of halogen, C₁₋₆ haloalkyl, C₁₋₆ alkyl, O—C₁₋₆ alkyl, O—C₁₋₆ haloalkyl, or C₃₋₆ cycloalkyl. In some embodiments, heteroaryl is optionally substituted with one or more substituents selected from the group consisting of halogen, C₁₋₆ haloalkyl, C₁₋₆ alkyl, O—C₁₋₆ alkyl, O—C₁₋₆ haloalkyl, or C₃₋₆ cycloalkyl. In some embodiments, the C₁₋₆ haloalkyl is CF₃, CHF₂, or CH₂F. In some embodiments, the O—C₁₋₆ haloalkyl is OCF₃, OCHF₂, or OCH₂F. In some embodiments, cycloalkyl is optionally substituted with halogen, C₁₋₆ alkyl, or O—C₁₋₆ alkyl.

In some embodiments of Formula (I), R⁴ and R⁵ together with the atom to which they are attached form a cycloalkyl or heterocyclyl. In some embodiments, R⁴ and R⁵ together with the atom to which they are attached form a cycloalkyl or heterocyclyl, each of which is optionally substituted. In some embodiments, the cycloalkyl or heterocyclyl is optionally substituted with —NS(O₂)(alkyl)(aryl). In some embodiments, the alkyl is C₁₋₅ alkyl and the aryl is phenyl optionally substituted with one or more halogen atoms. In some embodiments, the heterocyclyl is a 4- to 10-membered heterocyclyl. In some embodiments the heterocyclyl is a saturated 4- to 7-membered heterocyclyl.

In some embodiments of Formula (I), n is 0 and R⁴ and R⁵ together with the atom to which they are attached form an optionally substituted heterocyclyl selected from the group consisting of.

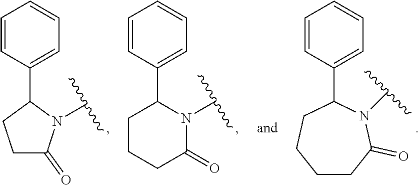

In some embodiments, the optionally substituted heterocyclyl is

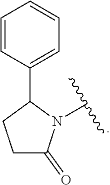 [SMILES: *N1C(=O)CCC1c1ccccc1]

In some embodiments, the optionally substituted heterocyclyl is

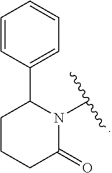 [SMILES: *N1C(=O)CCCC1c1ccccc1]

In some embodiments, the optionally substituted heterocyclyl is

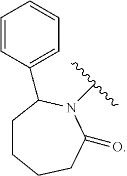 [SMILES: *N1C(=O)CCCCC1c1ccccc1]

In some embodiments of Formula (I) R¹ is selected from the group consisting of

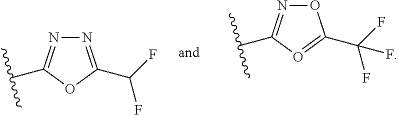

In some embodiments of Formula (I), R¹ is

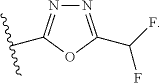 [SMILES: *c1nnc(C(F)F)o1]

In some embodiments, R¹ is

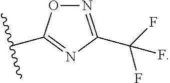 [SMILES: *c1nc(C(F)(F)F)no1]

In some embodiments, R¹ is

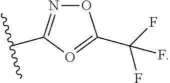

In some embodiments, R¹

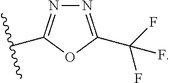 [SMILES: *c1nnc(C(F)(F)F)o1]

In some embodiments of Formula (I), R^{a} is H, halo, C₁₋₃ alkyl, or haloalkyl. In some embodiments, R^{a} is H. In some embodiments, R^{a} is C₁₋₃ alkyl. In some embodiments, R^{a} is haloalkyl. In some embodiments, halo is F. In some embodiments, the C₁₋₃ alkyl alkyl is methyl, ethyl or isopropyl. In some embodiments, haloalkyl is CF₃, CHF₂, or CH₂F.

In some embodiments of Formula (I), Y is CH and R⁴ and R⁵ are H.

In some embodiments of Formula (I), Y is N, R⁴ is H, and R⁵ is ethyl optionally substituted with —N(S(O₂)alkyl)(aryl) or —N(S(O₂)cycloalkyl)(aryl). In some embodiments, alkyl is C₁₋₅ alkyl, cycloalkyl is C₃₋₆ cycloalkyl, and aryl is phenyl optionally substituted with one or more halogen atoms.

In some embodiments of Formula (I), n is 1, X¹ is O or N, Y is N, R¹ is

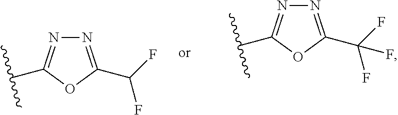 [SMILES: *c1nnc(C(F)F)o1 ; *c1nnc(C(F)(F)F)o1]

R² and R³ are H, R⁴ is H, —C₁₋₅ alkyl, —C(O)alkyl, —C(O)cycloalkyl, —(SO₂)NR²R³, —SO₂ alkyl, —SO₂ haloalkyl and —SO₂ cycloalkyl, each of which is optionally substituted, and R⁵ is aryl, heteroaryl, or cycloalkyl, each of which is optionally substituted.

In some embodiments of Formula (I), n is 1, X¹ is O or N, Y is O, R¹ is

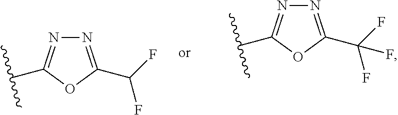 [SMILES: *c1nnc(C(F)F)o1 ; *c1nnc(C(F)(F)F)o1]

R² and R³ are H, and R⁵ is aryl, heteroaryl, cycloalkyl, or alkylenecycloalkyl, each of which is optionally substituted.

In some embodiments of Formula (I), n is 0, X¹ is O or N, Y is N, R¹ is

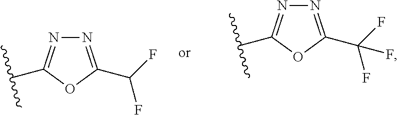 [SMILES: *c1nnc(C(F)F)o1 ; *c1nnc(C(F)(F)F)o1]

and R⁴ and R⁵ taken together with the atom to which they are attached form a cycloalkyl or heterocyclyl, each of which is optionally substituted.

In some embodiments, the present disclosure provides a compound of Formula (Ia) or a pharmaceutically acceptable salt thereof:

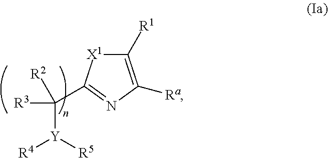

wherein:
R¹, R², R³, R⁴, R⁵, R^{a}, X¹, n, and Y are as defined above for Formula (I).

In some embodiments of Formula (Ia), R¹ is

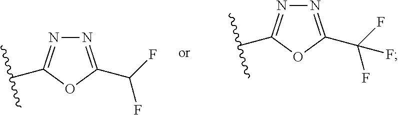 [SMILES: *c1nnc(C(F)F)o1 ; *c1nnc(C(F)(F)F)o1]

n is 1; Y is N; X¹ is S or O; and variables R², R³, R⁴, R⁵, and R^{a} are as defined above for Formula (I).

In some embodiments of Formula (Ia), n is 1, X¹ is S, Y is N, R¹ is

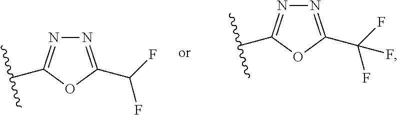 [SMILES: *c1nnc(C(F)F)o1 ; *c1nnc(C(F)(F)F)o1]

R² and R³ are H, R⁴ is —SO₂ alkyl, —SO₂ haloalkyl, or —SO₂ cycloalkyl, each of which is optionally substituted, R⁵ is heteroaryl, each of which is optionally substituted, and R^{a} is H or F. In some further embodiments, R⁴ is —SO₂C₁₋₅ alkyl, —SO₂ cyclopropyl, —SO₂CF₃ or —SO₂CHF₂, and the heteroaryl is optionally substituted pyridine or pyrazine. In some further embodiments, the heteroaryl is optionally substituted pyridine.

In some embodiments of Formula (Ia), n is 1, X¹ is S, Y is N, R¹ is

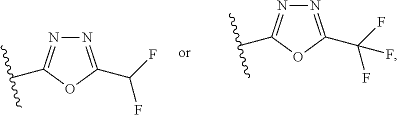 [SMILES: *c1nnc(C(F)F)o1 ; *c1nnc(C(F)(F)F)o1]

R² and R³ are H, R⁴ is —SO₂Me, —SO₂Et, or —SO₂ cyclopropyl, each of which is optionally substituted, R⁵ is pyridine or pyrazine, each of which is optionally substituted, and R^{a} is H. In some embodiments, R⁵ is optionally substituted pyridine.

In some embodiments of Formula (Ia), n is 1, X¹ is S, Y is N, R¹ is

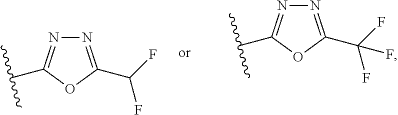 [SMILES: *c1nnc(C(F)F)o1 ; *c1nnc(C(F)(F)F)o1]

R² and R³ are H, R⁴ is —SO₂ alkyl or —SO₂ cycloalkyl, each of which is optionally substituted, R⁵ is

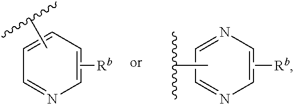

wherein R^{b} is selected from the group consisting of halogen, —C₁₋₅ alkyl, haloalkyl, —OC₁₋₅ alkyl, —Ohaloalkyl, —CH₂Ohaloalkyl, cyclopropyl, and CN, and R^{a} is H. In some embodiments, the halogen is F or Cl. In some embodiments, the haloalkyl is CF₃, CHF₂, CH₂CF₃, or CF₂CH₃. In some embodiments, the —C₁₋₅ alkyl is methyl.

In some embodiments of Formula (Ia), n is 1, X¹ is S, Y is N, R¹ is

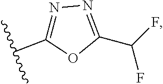 [SMILES: *c1nnc(C(F)F)o1]

R² and R³ are H, R⁴ is —SO₂Me, —SO₂Et, or —SO₂ cyclopropyl, each of which is optionally substituted, and R⁵ is

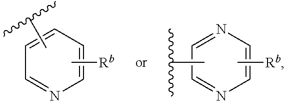

wherein R^{b} is selected from the group consisting of halogen, —C₁₋₅ alkyl, haloalkyl, —OC₁₋₅ alkyl, —Ohaloalkyl, —CH₂Ohaloalkyl, cyclopropyl, or CN, and R^{a} is H. In some embodiments, the halogen is F or Cl. In some embodiments, the haloalkyl is CF₃, CHF₂, CH₂CF₃, or CF₂CH₃. In some embodiments, the —C₁₋₅ alkyl is methyl.

In some embodiments of Formula (Ia), n is 1, X¹ is S, Y is N, R¹ is

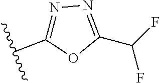 [SMILES: *c1nnc(C(F)F)o1]

R² and R³ are H, R⁴ is —SO₂Me, —SO₂Et, or —SO₂ cyclopropyl, each of which is optionally substituted, and R⁵ is

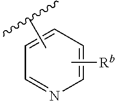

wherein R^{b} is selected from the group consisting of Cl, F, Me, cyclopropyl, CF₃, CHF₂, CF₂CH₃, OCF₃, OCHF₂, OCH₂CF₂H and CN, and R^{a} is H.

In some embodiments, the present disclosure provides a compound of Formula (Ib) or a pharmaceutically acceptable salt thereof:

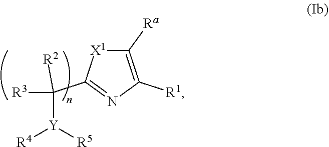

wherein: R¹, R², R³, R⁴, R⁵, R^{a}, X¹, n, and Y are as defined above for Formula (I).

In some embodiments of Formulas (I)-(Ib), each optionally substituted alkyl is independently an optionally substituted C₁₋₆ alkyl. In some embodiments, the C₁₋₆ alkyl is Me or Et.

In some embodiments of Formulas (I)-(Ib), each optionally substituted haloalkyl is independently an optionally substituted C₁₋₆ haloalkyl. In some embodiments, the C₁₋₆ haloalkyl is CF₃, CHF₂, or CH₂F. In some embodiments, the C₁₋₆ haloalkyl is CF₃ or CHF₂.

In some embodiments of Formulas (I)-(Ib), each optionally substituted cycloalkyl is independently an optionally substituted C₃₋₁₂ cycloalkyl. In some embodiments, the cycloalkyl is a C₃₋₆ cycloalkyl. In some embodiments, the cycloalkyl is selected from the group consisting of cyclopropyl, cyclobutyl, cyclopentyl, and cyclohexyl.

In some embodiments of Formulas (I)-(Ib), each optionally substituted heterocyclyl is independently an optionally substituted 3-12 membered heterocycloalkyl having 1 or 2 heteroatoms independently selected from N, O, and S. In some embodiments, each optionally substituted heterocyclyl is independently an optionally substituted 3-6 membered heterocycloalkyl having 1 or 2 heteroatoms independently selected from N, O, and S. In further embodiments, the heterocycloalkyl is an optionally substituted 5-membered or 6-membered heterocycle having 1 or 2 heteroatoms independently selected from N, O, and S. In some embodiments, the heterocyclyl is selected from the group consisting of aziridinyl, azetidinyl, pyrrolidinyl, piperidinyl, and morpholinyl, and thiomorpholinyl.

In some embodiments of Formulas (I)-(Ib), each optionally substituted aryl is independently a C₆₋₁₂ aryl. In further embodiments, the C₆₋₁₂ aryl is an optionally substituted phenyl.

In some embodiments of Formulas (I)-(Ib), each optionally substituted heteroaryl is independently a 5-12 membered heteroaryl having 1, 2, or 3 heteroatoms independently selected from N, O, and S. In some embodiments, each optionally substituted heteroaryl is independently a 5-12 membered heteroaryl having 3 heteroatoms independently selected from N, O, and S. In some embodiments, each optionally substituted heteroaryl is independently a 5-12 membered heteroaryl having 2 heteroatoms independently selected from N, O, and S. In some embodiments, each optionally substituted heteroaryl is independently a 5-12 membered heteroaryl having 1 heteroatom independently selected from N, O, and S. In further embodiments, each optionally substituted heteroaryl is an optionally substituted 5-membered or 6-membered heteroaryl having 1 heteroatom independently from N, O, and S. In some embodiments, each heteroaryl is independently selected from the group consisting of tetrazole, oxadiazole, thiadiazole, imidazole, pyrazole, thiazole, or oxazole, each of which is optionally substituted.

In some embodiments, the compound of Formula (I) is selected from the group consisting of

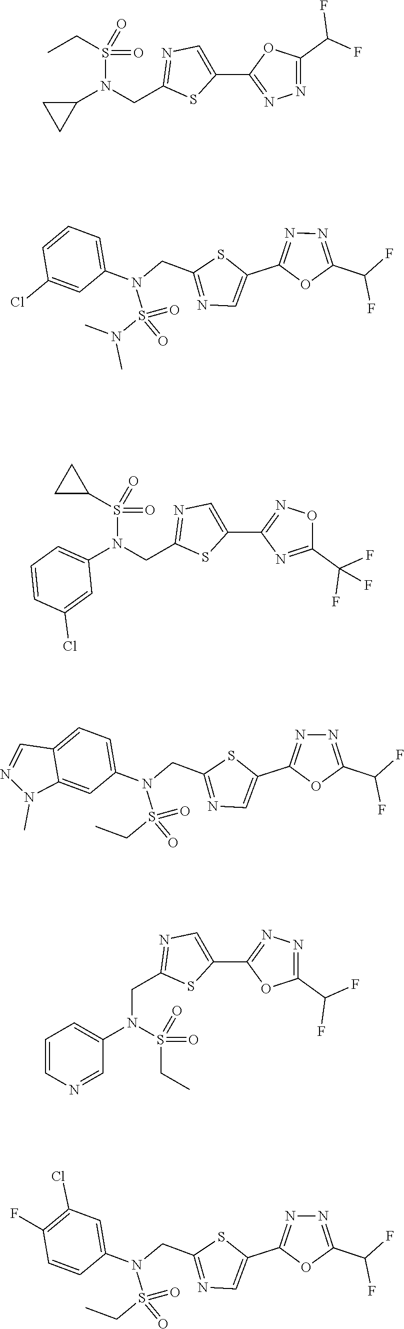 [SMILES: CCS(=O)(=O)N(Cc1ncc(-c2nnc(C(F)F)o2)s1)C1CC1 ; CN(C)S(=O)(=O)N(Cc1ncc(-c2nnc(C(F)F)o2)s1)c1cccc(Cl)c1 ; O=S(=O)(C1CC1)N(Cc1ncc(-c2noc(C(F)(F)F)n2)s1)c1cccc(Cl)c1 ; CCS(=O)(=O)N(Cc1ncc(-c2nnc(C(F)F)o2)s1)c1ccc2cnn(C)c2c1 ; CCS(=O)(=O)N(Cc1ncc(-c2nnc(C(F)F)o2)s1)c1cccnc1 ; CCS(=O)(=O)N(Cc1ncc(-c2nnc(C(F)F)o2)s1)c1ccc(F)c(Cl)c1]

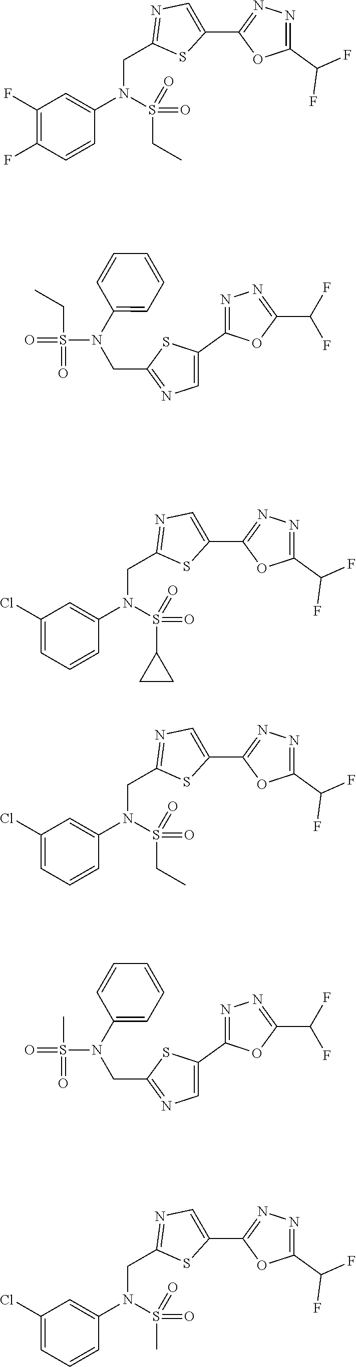 [SMILES: CCS(=O)(=O)N(Cc1ncc(-c2nnc(C(F)F)o2)s1)c1ccc(F)c(F)c1 ; CCS(=O)(=O)N(Cc1ncc(-c2nnc(C(F)F)o2)s1)c1ccccc1 ; O=S(=O)(C1CC1)N(Cc1ncc(-c2nnc(C(F)F)o2)s1)c1cccc(Cl)c1 ; CCS(=O)(=O)N(Cc1ncc(-c2nnc(C(F)F)o2)s1)c1cccc(Cl)c1 ; CS(=O)(=O)N(Cc1ncc(-c2nnc(C(F)F)o2)s1)c1ccccc1 ; CS(=O)(=O)N(Cc1ncc(-c2nnc(C(F)F)o2)s1)c1cccc(Cl)c1]

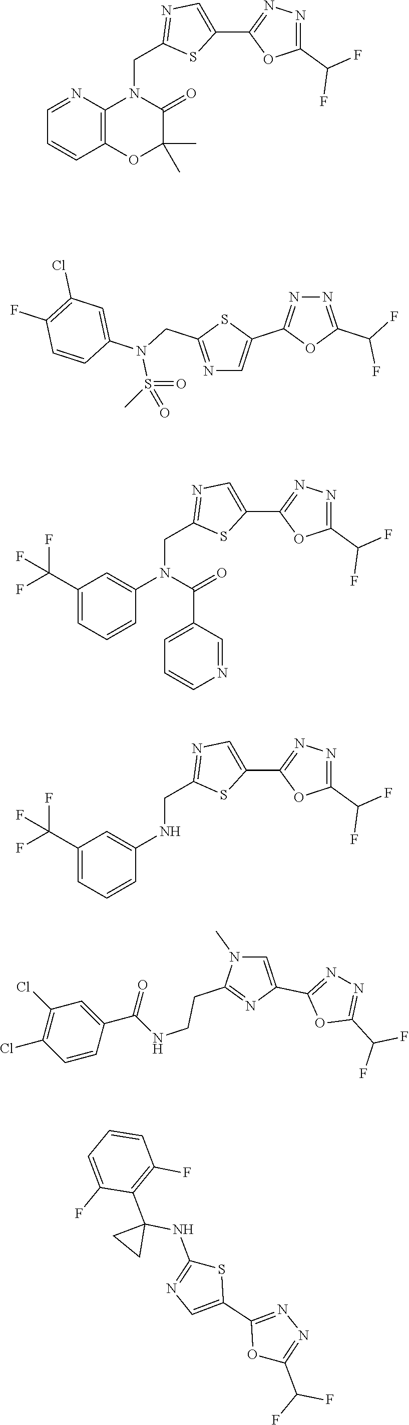 [SMILES: CC1(C)Oc2cccnc2N(Cc2ncc(-c3nnc(C(F)F)o3)s2)C1=O ; CS(=O)(=O)N(Cc1ncc(-c2nnc(C(F)F)o2)s1)c1ccc(F)c(Cl)c1 ; O=C(c1cccnc1)N(Cc1ncc(-c2nnc(C(F)F)o2)s1)c1cccc(C(F)(F)F)c1 ; FC(F)c1nnc(-c2cnc(CNc3cccc(C(F)(F)F)c3)s2)o1 ; Cn1cc(-c2nnc(C(F)F)o2)nc1CCNC(=O)c1ccc(Cl)c(Cl)c1 ; Fc1cccc(F)c1C1(Nc2ncc(-c3nnc(C(F)F)o3)s2)CC1]

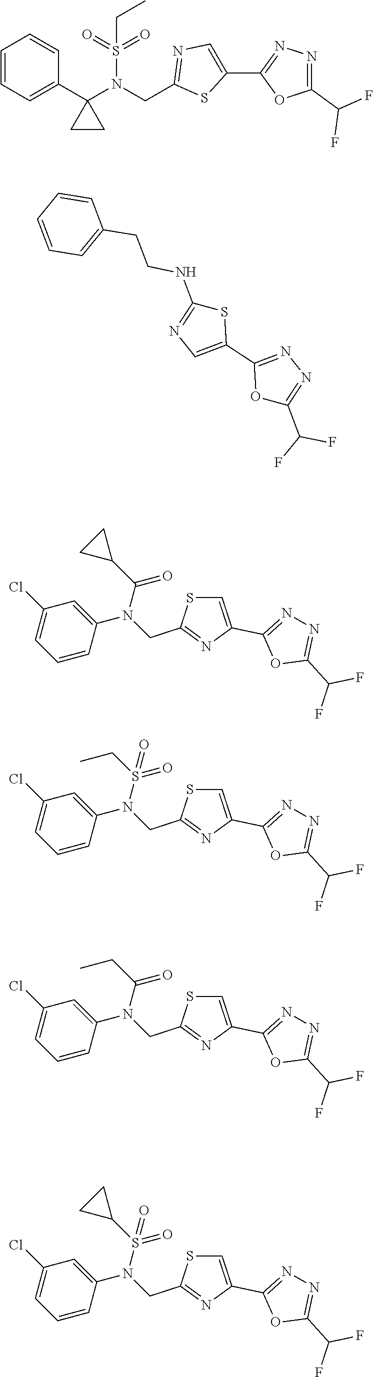 [SMILES: CCS(=O)(=O)N(Cc1ncc(-c2nnc(C(F)F)o2)s1)C1(c2ccccc2)CC1 ; FC(F)c1nnc(-c2cnc(NCCc3ccccc3)s2)o1 ; O=C(C1CC1)N(Cc1nc(-c2nnc(C(F)F)o2)cs1)c1cccc(Cl)c1 ; CCS(=O)(=O)N(Cc1nc(-c2nnc(C(F)F)o2)cs1)c1cccc(Cl)c1 ; CCC(=O)N(Cc1nc(-c2nnc(C(F)F)o2)cs1)c1cccc(Cl)c1 ; O=S(=O)(C1CC1)N(Cc1nc(-c2nnc(C(F)F)o2)cs1)c1cccc(Cl)c1]

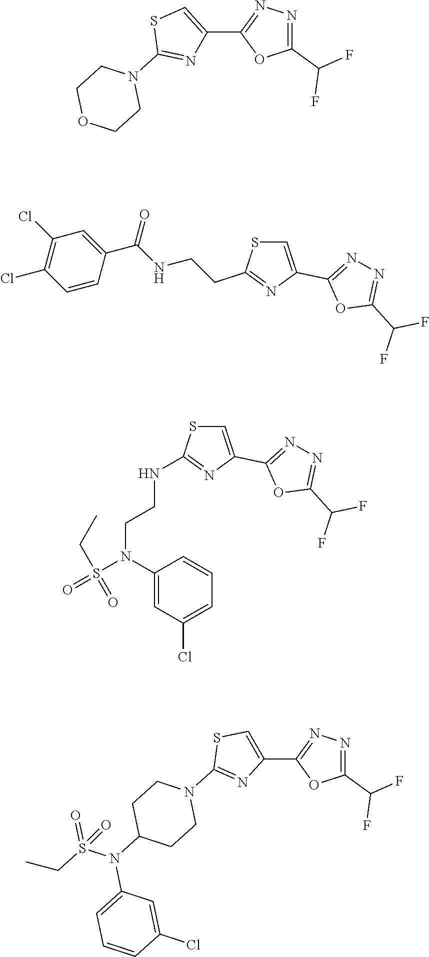 [SMILES: FC(F)c1nnc(-c2csc(N3CCOCC3)n2)o1 ; O=C(NCCc1nc(-c2nnc(C(F)F)o2)cs1)c1ccc(Cl)c(Cl)c1 ; CCS(=O)(=O)N(CCNc1nc(-c2nnc(C(F)F)o2)cs1)c1cccc(Cl)c1 ; CCS(=O)(=O)N(c1cccc(Cl)c1)C1CCN(c2nc(-c3nnc(C(F)F)o3)cs2)CC1]

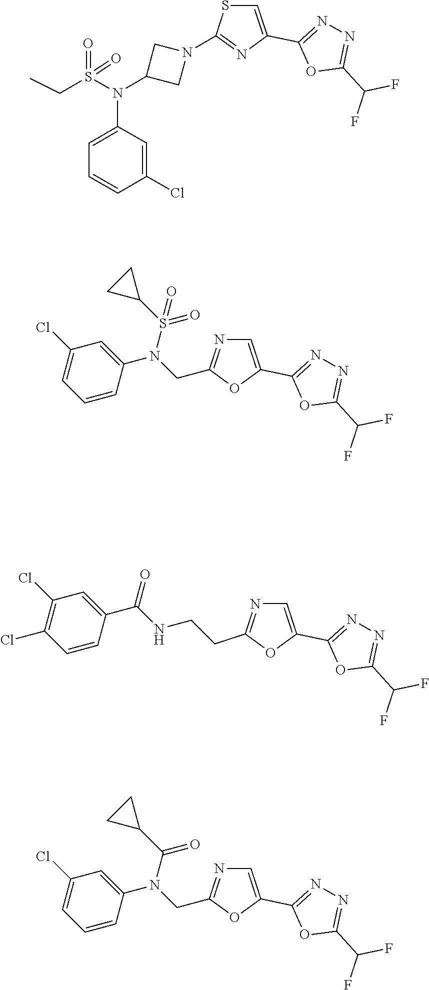 [SMILES: CCS(=O)(=O)N(c1cccc(Cl)c1)C1CN(c2nc(-c3nnc(C(F)F)o3)cs2)C1 ; O=S(=O)(C1CC1)N(Cc1ncc(-c2nnc(C(F)F)o2)o1)c1cccc(Cl)c1 ; O=C(NCCc1ncc(-c2nnc(C(F)F)o2)o1)c1ccc(Cl)c(Cl)c1 ; O=C(C1CC1)N(Cc1ncc(-c2nnc(C(F)F)o2)o1)c1cccc(Cl)c1]

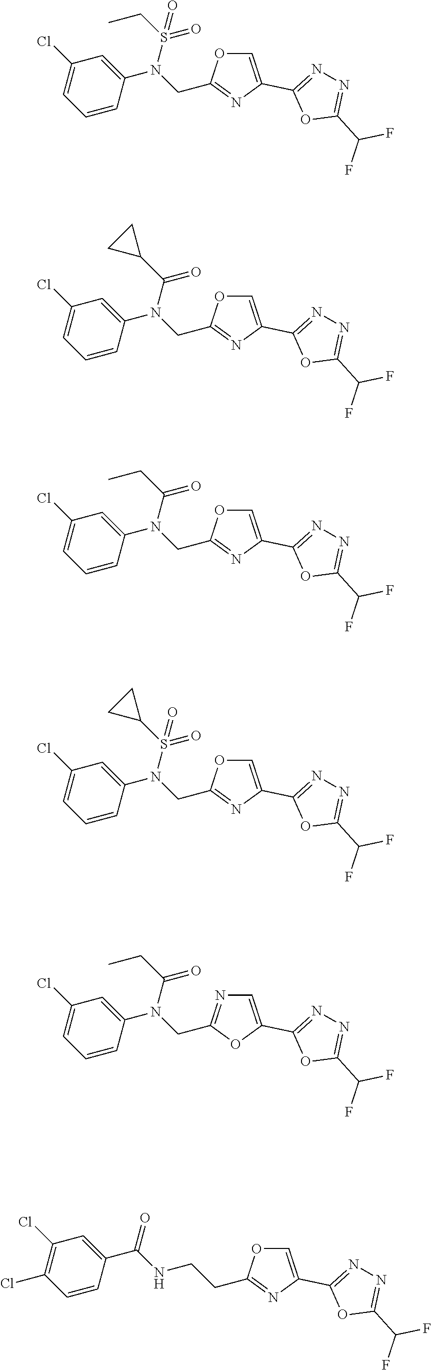 [SMILES: CCS(=O)(=O)N(Cc1nc(-c2nnc(C(F)F)o2)co1)c1cccc(Cl)c1 ; O=C(C1CC1)N(Cc1nc(-c2nnc(C(F)F)o2)co1)c1cccc(Cl)c1 ; CCC(=O)N(Cc1nc(-c2nnc(C(F)F)o2)co1)c1cccc(Cl)c1 ; O=S(=O)(C1CC1)N(Cc1nc(-c2nnc(C(F)F)o2)co1)c1cccc(Cl)c1 ; CCC(=O)N(Cc1ncc(-c2nnc(C(F)F)o2)o1)c1cccc(Cl)c1 ; O=C(NCCc1nc(-c2nnc(C(F)F)o2)co1)c1ccc(Cl)c(Cl)c1]

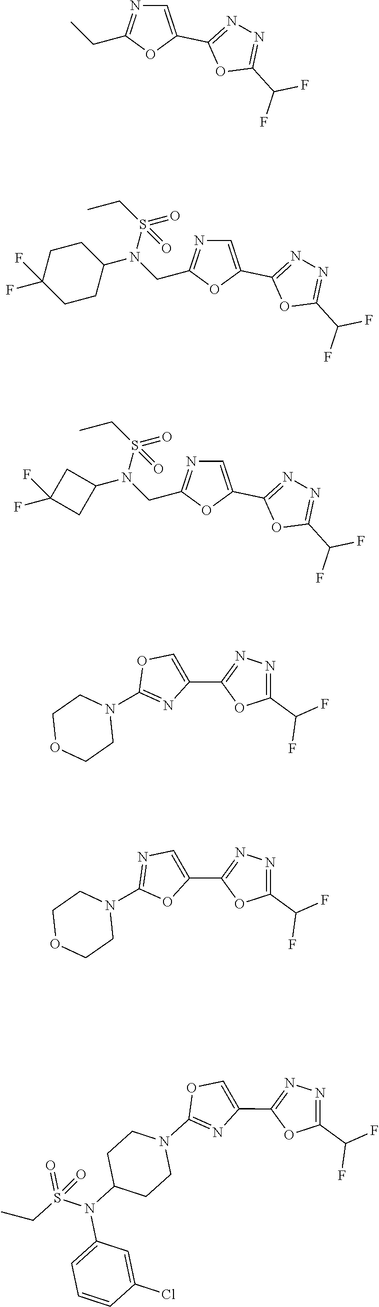 [SMILES: CCc1ncc(-c2nnc(C(F)F)o2)o1 ; CCS(=O)(=O)N(Cc1ncc(-c2nnc(C(F)F)o2)o1)C1CCC(F)(F)CC1 ; CCS(=O)(=O)N(Cc1ncc(-c2nnc(C(F)F)o2)o1)C1CC(F)(F)C1 ; FC(F)c1nnc(-c2coc(N3CCOCC3)n2)o1 ; FC(F)c1nnc(-c2cnc(N3CCOCC3)o2)o1 ; CCS(=O)(=O)N(c1cccc(Cl)c1)C1CCN(c2nc(-c3nnc(C(F)F)o3)co2)CC1]

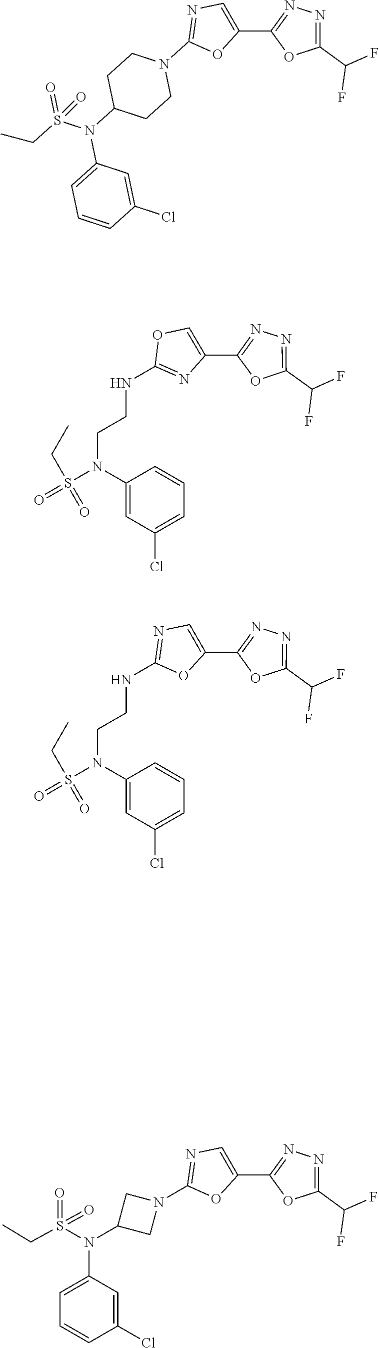 [SMILES: CCS(=O)(=O)N(c1cccc(Cl)c1)C1CCN(c2ncc(-c3nnc(C(F)F)o3)o2)CC1 ; CCS(=O)(=O)N(CCNc1nc(-c2nnc(C(F)F)o2)co1)c1cccc(Cl)c1 ; CCS(=O)(=O)N(CCNc1ncc(-c2nnc(C(F)F)o2)o1)c1cccc(Cl)c1 ; CCS(=O)(=O)N(c1cccc(Cl)c1)C1CN(c2ncc(-c3nnc(C(F)F)o3)o2)C1]

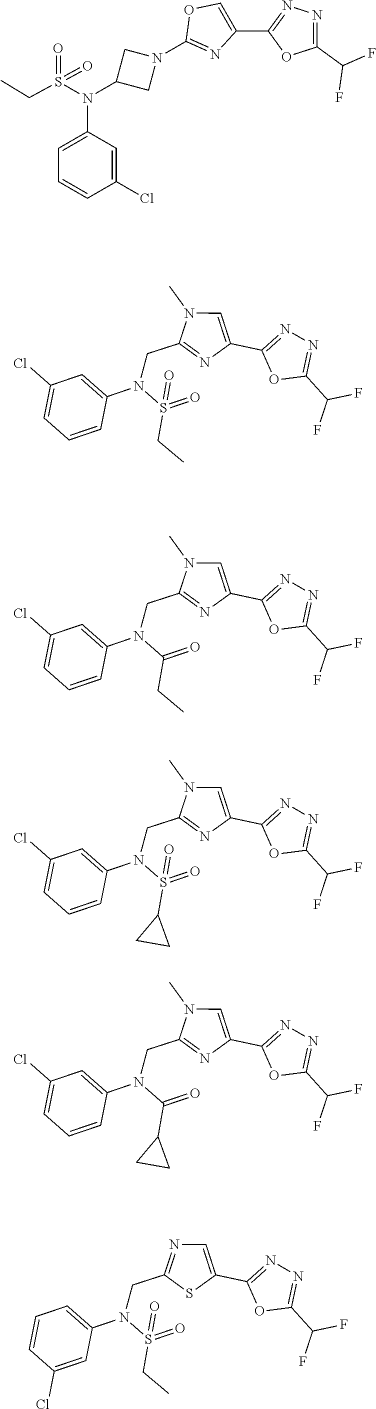

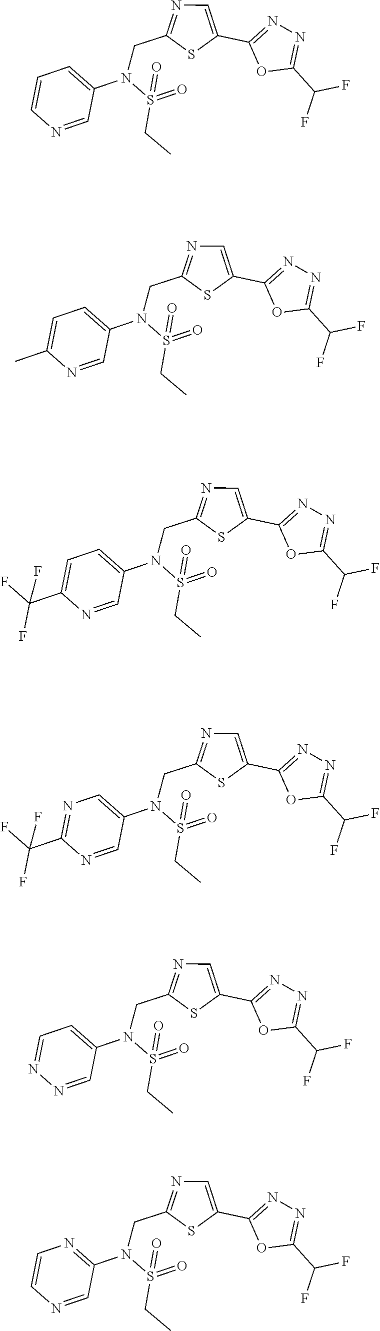 [SMILES: CCS(=O)(=O)N(Cc1ncc(-c2nnc(C(F)F)o2)s1)c1cccnc1 ; CCS(=O)(=O)N(Cc1ncc(-c2nnc(C(F)F)o2)s1)c1ccc(C)nc1 ; CCS(=O)(=O)N(Cc1ncc(-c2nnc(C(F)F)o2)s1)c1ccc(C(F)(F)F)nc1 ; CCS(=O)(=O)N(Cc1ncc(-c2nnc(C(F)F)o2)s1)c1cnc(C(F)(F)F)nc1 ; CCS(=O)(=O)N(Cc1ncc(-c2nnc(C(F)F)o2)s1)c1ccnnc1 ; CCS(=O)(=O)N(Cc1ncc(-c2nnc(C(F)F)o2)s1)c1cnccn1]

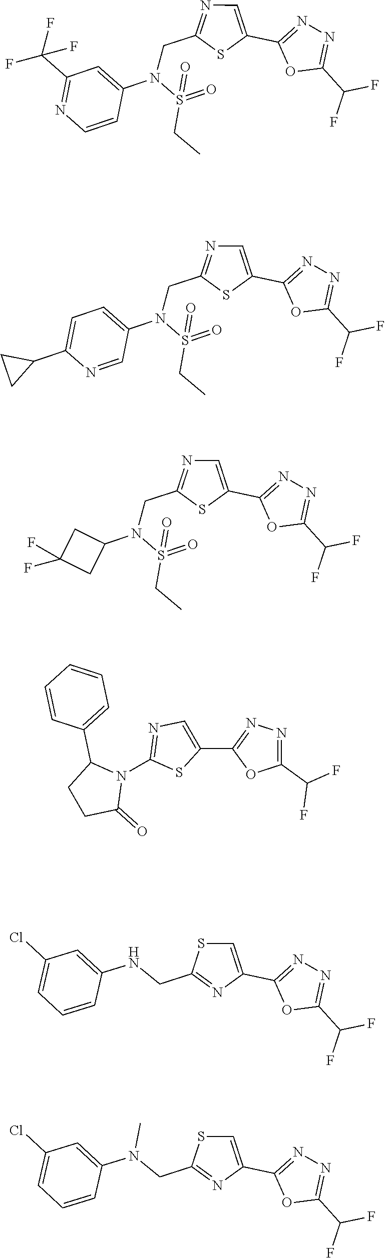 [SMILES: CCS(=O)(=O)N(Cc1ncc(-c2nnc(C(F)F)o2)s1)c1ccnc(C(F)(F)F)c1 ; CCS(=O)(=O)N(Cc1ncc(-c2nnc(C(F)F)o2)s1)c1ccc(C2CC2)nc1 ; CCS(=O)(=O)N(Cc1ncc(-c2nnc(C(F)F)o2)s1)C1CC(F)(F)C1 ; O=C1CCC(c2ccccc2)N1c1ncc(-c2nnc(C(F)F)o2)s1 ; FC(F)c1nnc(-c2csc(CNc3cccc(Cl)c3)n2)o1 ; CN(Cc1nc(-c2nnc(C(F)F)o2)cs1)c1cccc(Cl)c1]

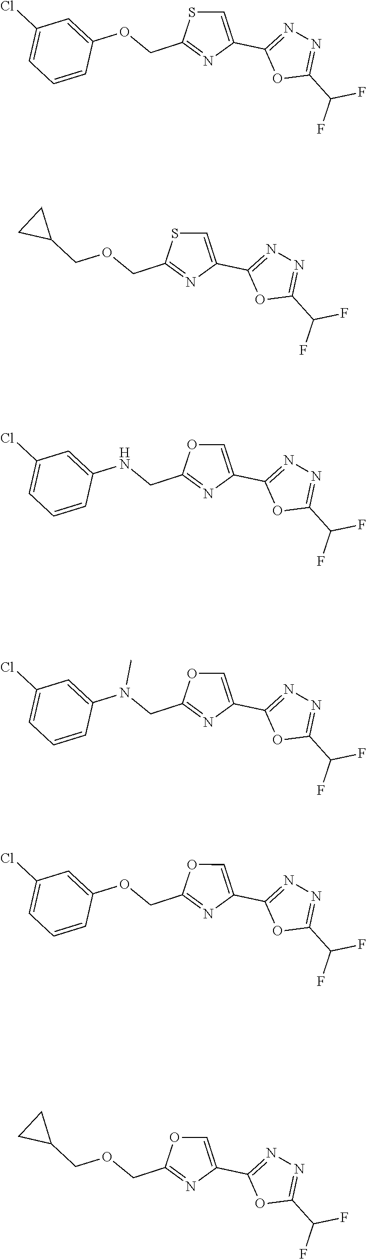 [SMILES: FC(F)c1nnc(-c2csc(COc3cccc(Cl)c3)n2)o1 ; FC(F)c1nnc(-c2csc(COCC3CC3)n2)o1 ; FC(F)c1nnc(-c2coc(CNc3cccc(Cl)c3)n2)o1 ; CN(Cc1nc(-c2nnc(C(F)F)o2)co1)c1cccc(Cl)c1 ; FC(F)c1nnc(-c2coc(COc3cccc(Cl)c3)n2)o1 ; FC(F)c1nnc(-c2coc(COCC3CC3)n2)o1]

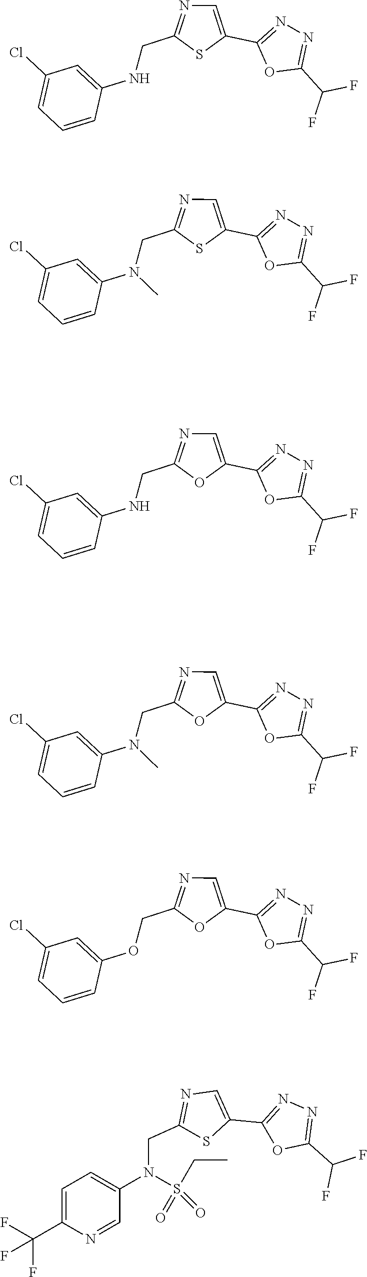 [SMILES: FC(F)c1nnc(-c2cnc(CNc3cccc(Cl)c3)s2)o1 ; CN(Cc1ncc(-c2nnc(C(F)F)o2)s1)c1cccc(Cl)c1 ; FC(F)c1nnc(-c2cnc(CNc3cccc(Cl)c3)o2)o1 ; CN(Cc1ncc(-c2nnc(C(F)F)o2)o1)c1cccc(Cl)c1 ; FC(F)c1nnc(-c2cnc(COc3cccc(Cl)c3)o2)o1 ; CCS(=O)(=O)N(Cc1ncc(-c2nnc(C(F)F)o2)s1)c1ccc(C(F)(F)F)nc1]

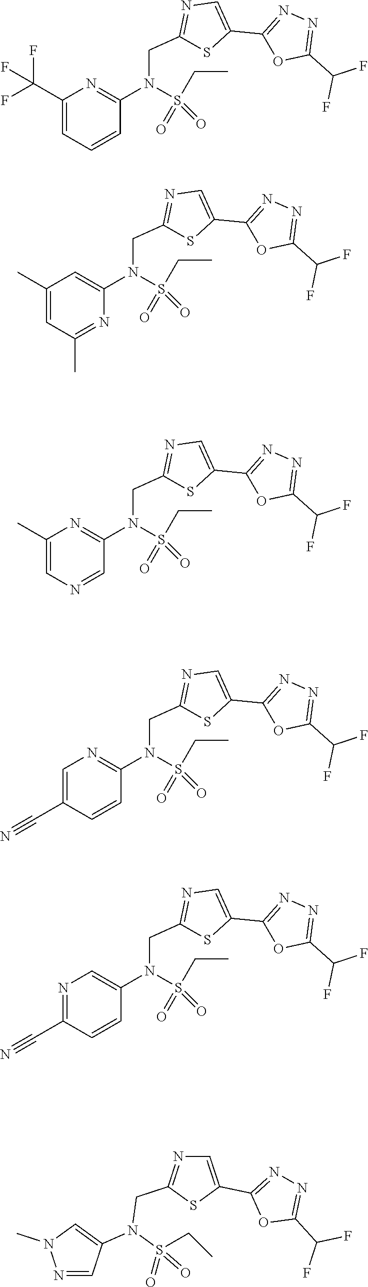 [SMILES: CCS(=O)(=O)N(Cc1ncc(-c2nnc(C(F)F)o2)s1)c1cccc(C(F)(F)F)n1 ; CCS(=O)(=O)N(Cc1ncc(-c2nnc(C(F)F)o2)s1)c1cc(C)cc(C)n1 ; CCS(=O)(=O)N(Cc1ncc(-c2nnc(C(F)F)o2)s1)c1cncc(C)n1 ; CCS(=O)(=O)N(Cc1ncc(-c2nnc(C(F)F)o2)s1)c1ccc(C#N)cn1 ; CCS(=O)(=O)N(Cc1ncc(-c2nnc(C(F)F)o2)s1)c1ccc(C#N)nc1 ; CCS(=O)(=O)N(Cc1ncc(-c2nnc(C(F)F)o2)s1)c1cnn(C)c1]

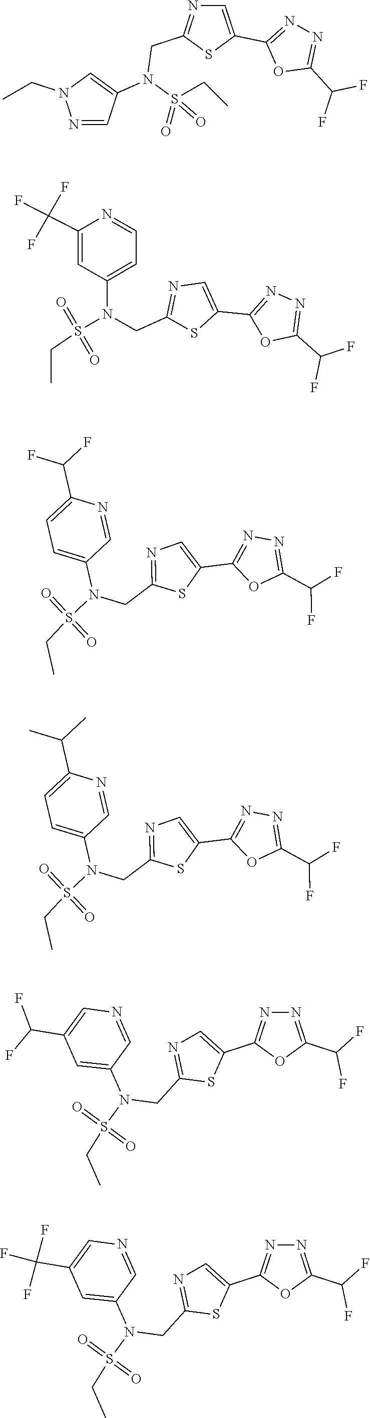 [SMILES: CCn1cc(N(Cc2ncc(-c3nnc(C(F)F)o3)s2)S(=O)(=O)CC)cn1 ; CCS(=O)(=O)N(Cc1ncc(-c2nnc(C(F)F)o2)s1)c1ccnc(C(F)(F)F)c1 ; CCS(=O)(=O)N(Cc1ncc(-c2nnc(C(F)F)o2)s1)c1ccc(C(F)F)nc1 ; CCS(=O)(=O)N(Cc1ncc(-c2nnc(C(F)F)o2)s1)c1ccc(C(C)C)nc1 ; CCS(=O)(=O)N(Cc1ncc(-c2nnc(C(F)F)o2)s1)c1cncc(C(F)F)c1 ; CCS(=O)(=O)N(Cc1ncc(-c2nnc(C(F)F)o2)s1)c1cncc(C(F)(F)F)c1]

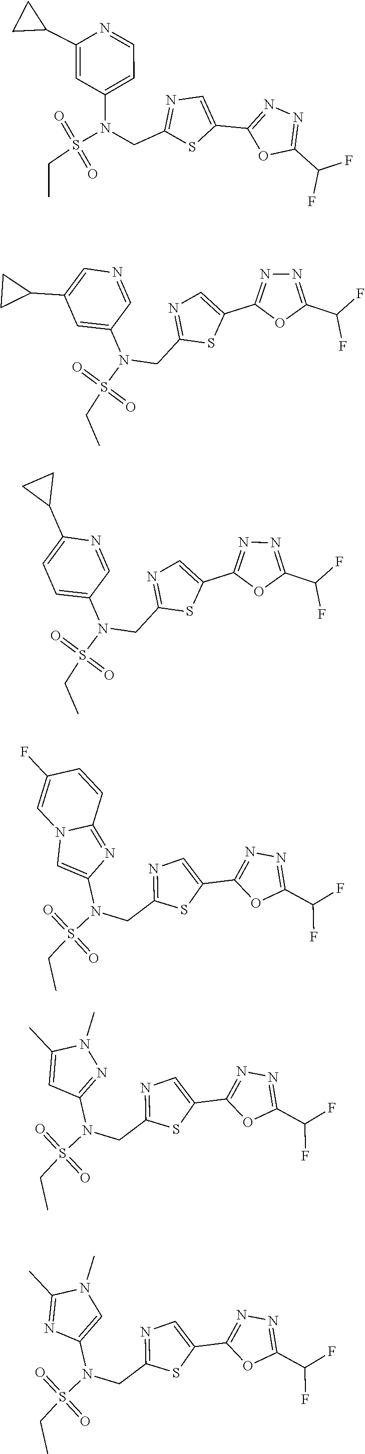

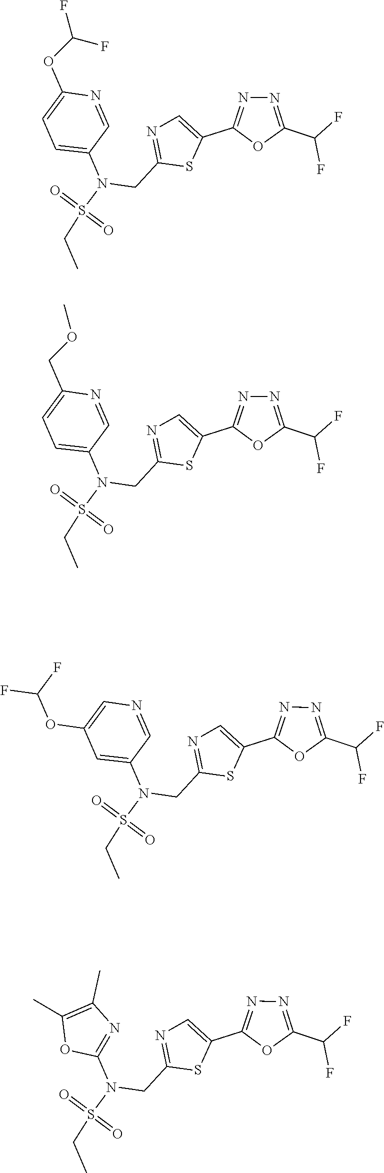

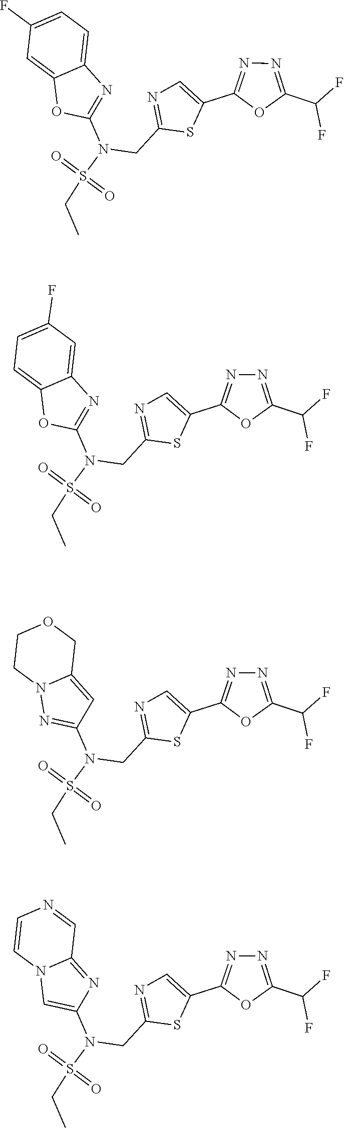 [SMILES: CCS(=O)(=O)N(Cc1ncc(-c2nnc(C(F)F)o2)s1)c1nc2ccc(F)cc2o1 ; CCS(=O)(=O)N(Cc1ncc(-c2nnc(C(F)F)o2)s1)c1nc2cc(F)ccc2o1 ; CCS(=O)(=O)N(Cc1ncc(-c2nnc(C(F)F)o2)s1)c1cc2n(n1)CCOC2 ; CCS(=O)(=O)N(Cc1ncc(-c2nnc(C(F)F)o2)s1)c1cn2ccncc2n1]

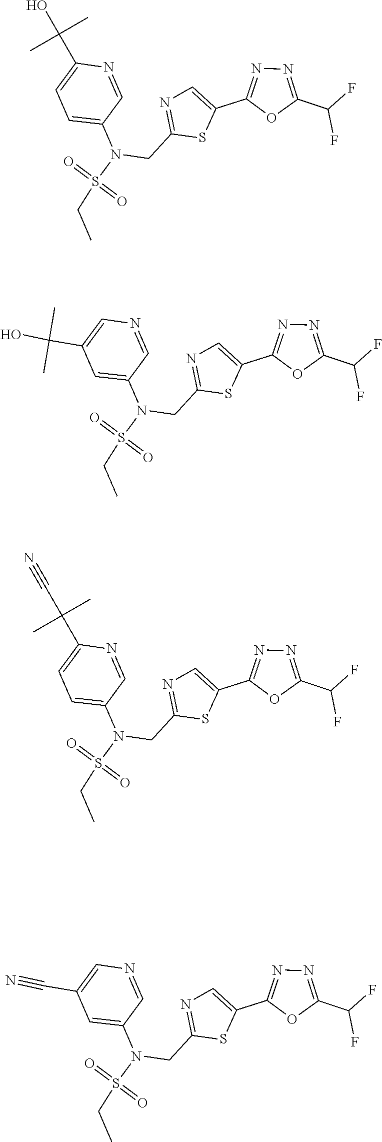 [SMILES: CCS(=O)(=O)N(Cc1ncc(-c2nnc(C(F)F)o2)s1)c1ccc(C(C)(C)O)nc1 ; CCS(=O)(=O)N(Cc1ncc(-c2nnc(C(F)F)o2)s1)c1cncc(C(C)(C)O)c1 ; CCS(=O)(=O)N(Cc1ncc(-c2nnc(C(F)F)o2)s1)c1ccc(C(C)(C)C#N)nc1 ; CCS(=O)(=O)N(Cc1ncc(-c2nnc(C(F)F)o2)s1)c1cncc(C#N)c1]

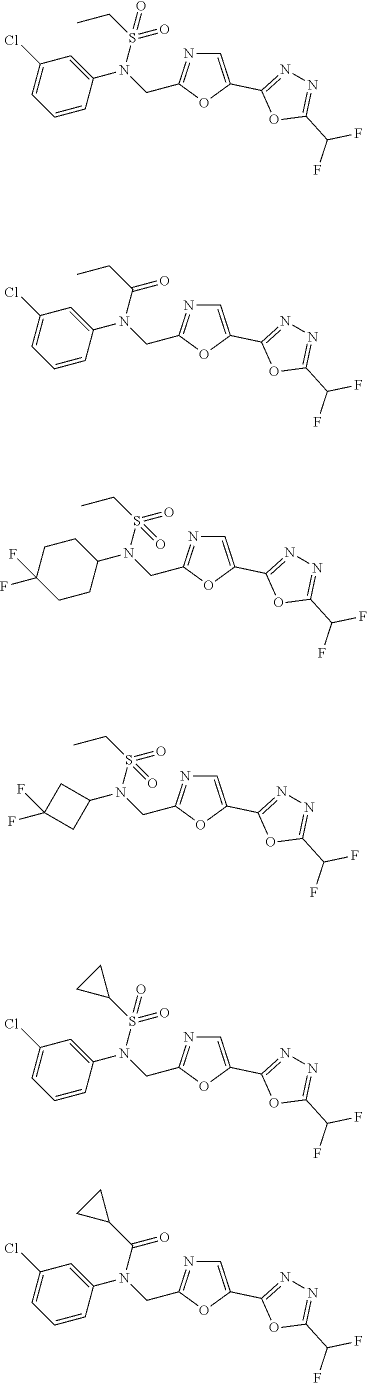 [SMILES: CCS(=O)(=O)N(Cc1ncc(-c2nnc(C(F)F)o2)o1)c1cccc(Cl)c1 ; CCC(=O)N(Cc1ncc(-c2nnc(C(F)F)o2)o1)c1cccc(Cl)c1 ; CCS(=O)(=O)N(Cc1ncc(-c2nnc(C(F)F)o2)o1)C1CCC(F)(F)CC1 ; CCS(=O)(=O)N(Cc1ncc(-c2nnc(C(F)F)o2)o1)C1CC(F)(F)C1 ; O=S(=O)(C1CC1)N(Cc1ncc(-c2nnc(C(F)F)o2)o1)c1cccc(Cl)c1 ; O=C(C1CC1)N(Cc1ncc(-c2nnc(C(F)F)o2)o1)c1cccc(Cl)c1]

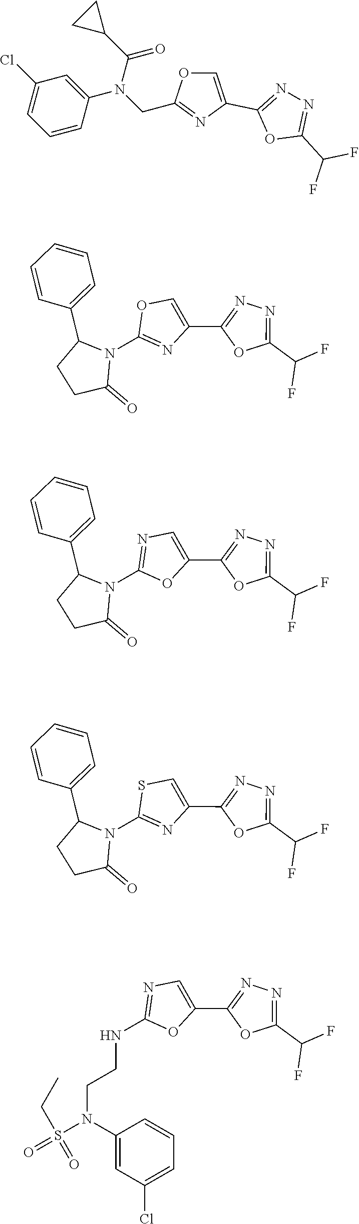 [SMILES: O=C(C1CC1)N(Cc1nc(-c2nnc(C(F)F)o2)co1)c1cccc(Cl)c1 ; O=C1CCC(c2ccccc2)N1c1nc(-c2nnc(C(F)F)o2)co1 ; O=C1CCC(c2ccccc2)N1c1ncc(-c2nnc(C(F)F)o2)o1 ; O=C1CCC(c2ccccc2)N1c1nc(-c2nnc(C(F)F)o2)cs1 ; CCS(=O)(=O)N(CCNc1ncc(-c2nnc(C(F)F)o2)o1)c1cccc(Cl)c1]

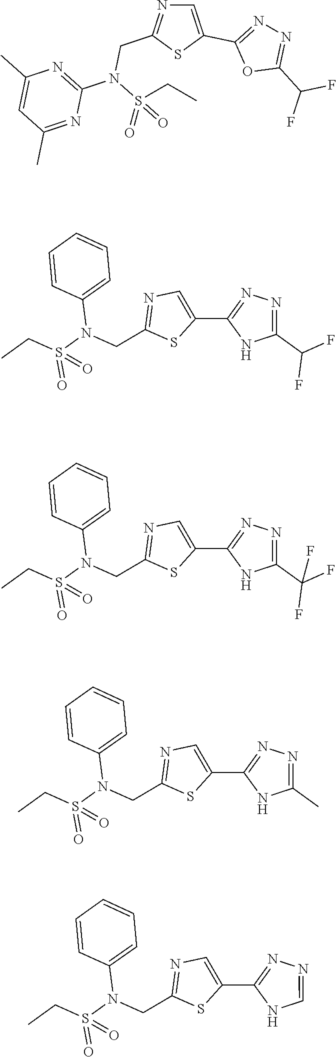

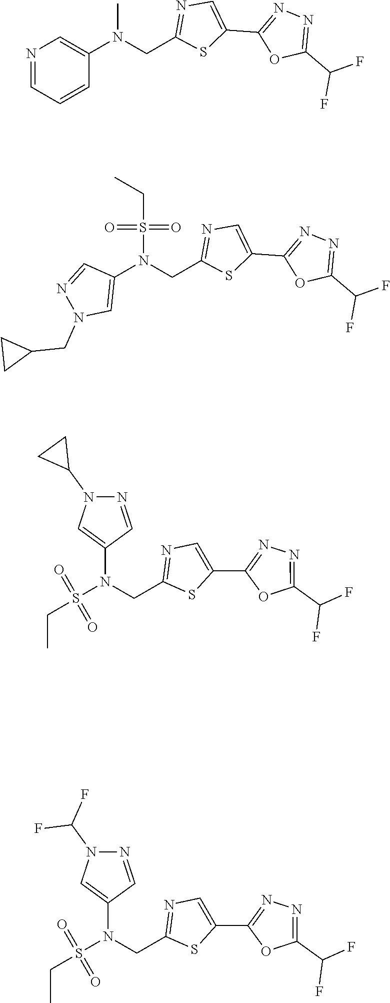 [SMILES: CN(Cc1ncc(-c2nnc(C(F)F)o2)s1)c1cccnc1 ; CCS(=O)(=O)N(Cc1ncc(-c2nnc(C(F)F)o2)s1)c1cnn(CC2CC2)c1 ; CCS(=O)(=O)N(Cc1ncc(-c2nnc(C(F)F)o2)s1)c1cnn(C2CC2)c1 ; CCS(=O)(=O)N(Cc1ncc(-c2nnc(C(F)F)o2)s1)c1cnn(C(F)F)c1]

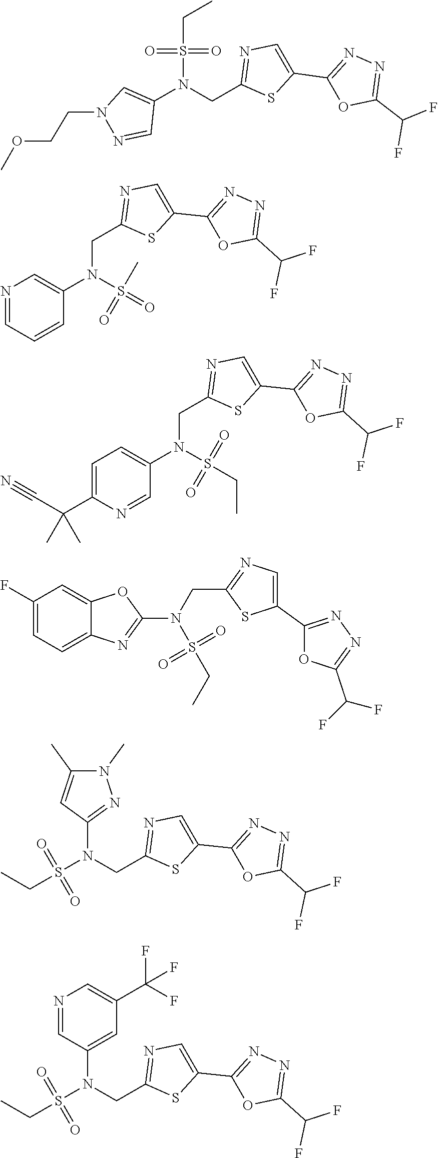

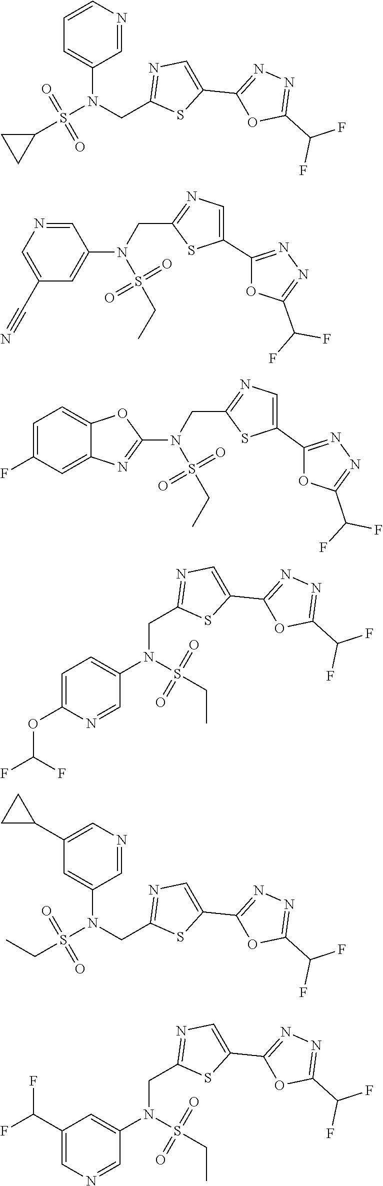

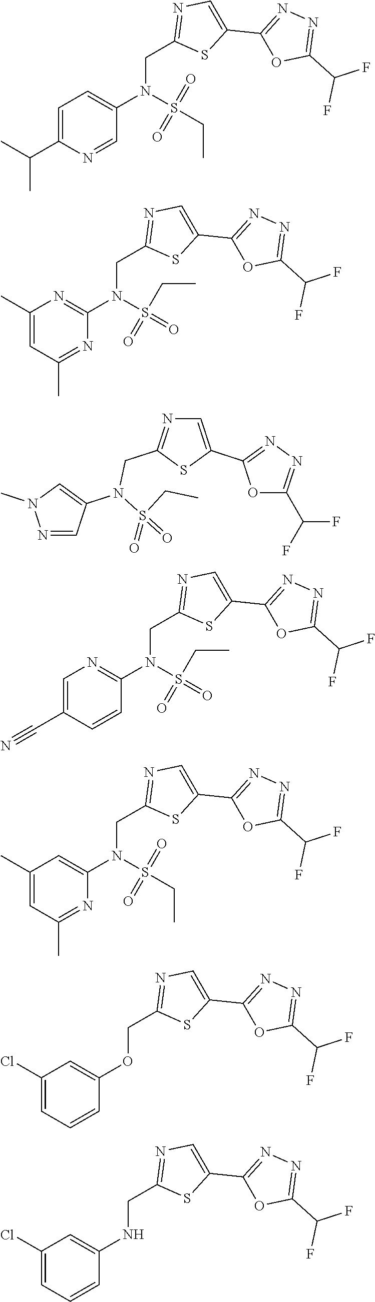

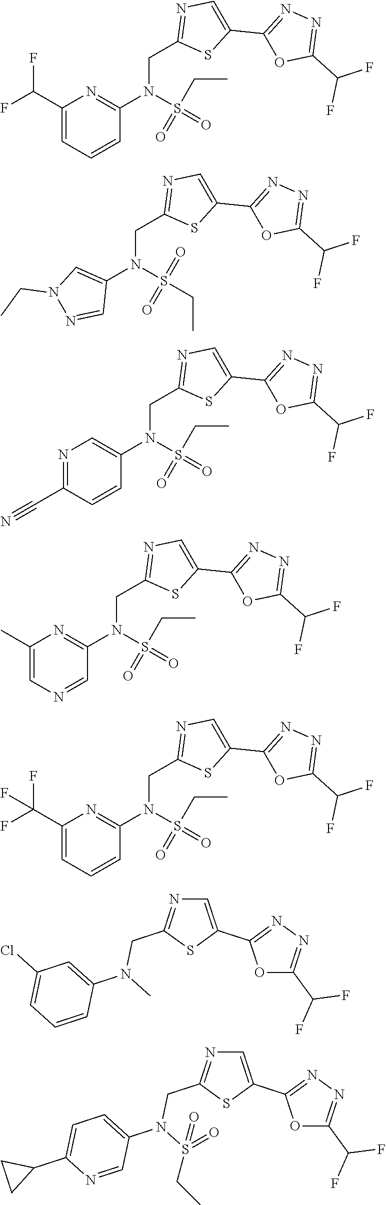

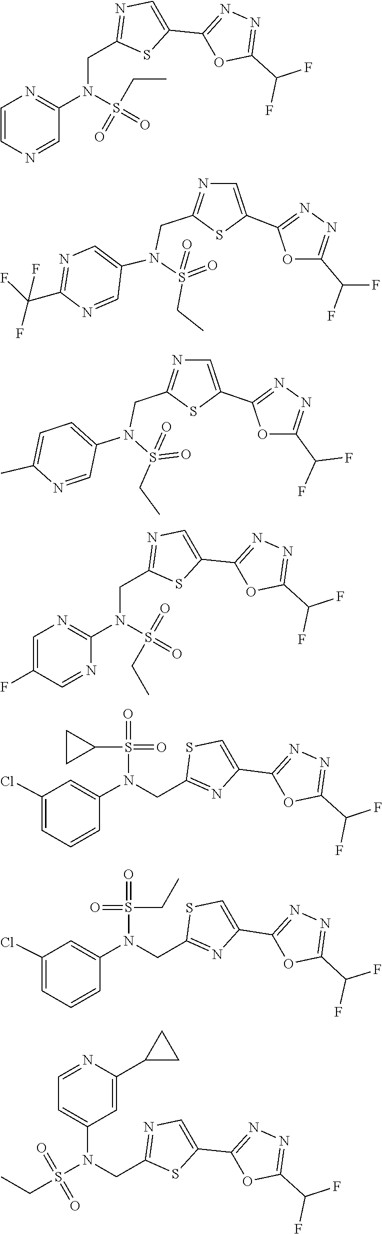

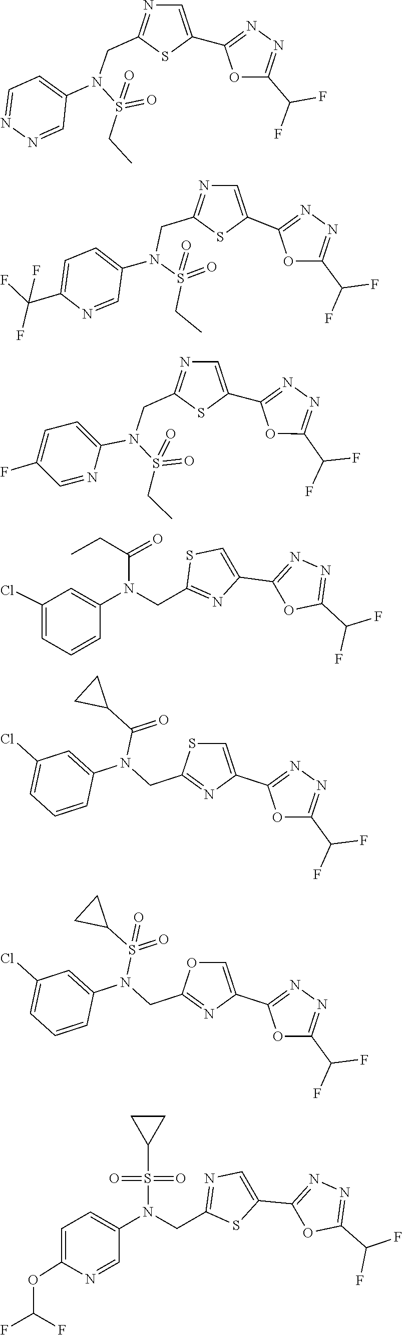

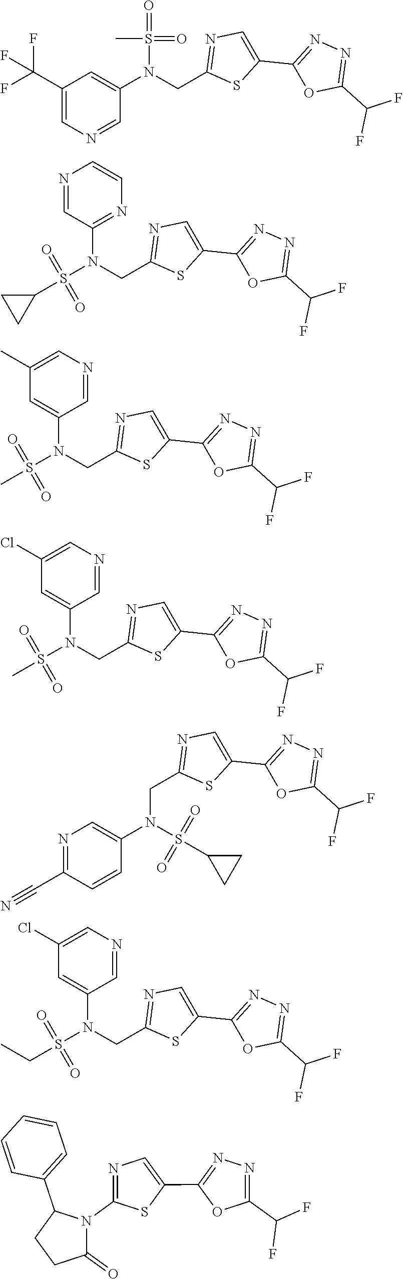

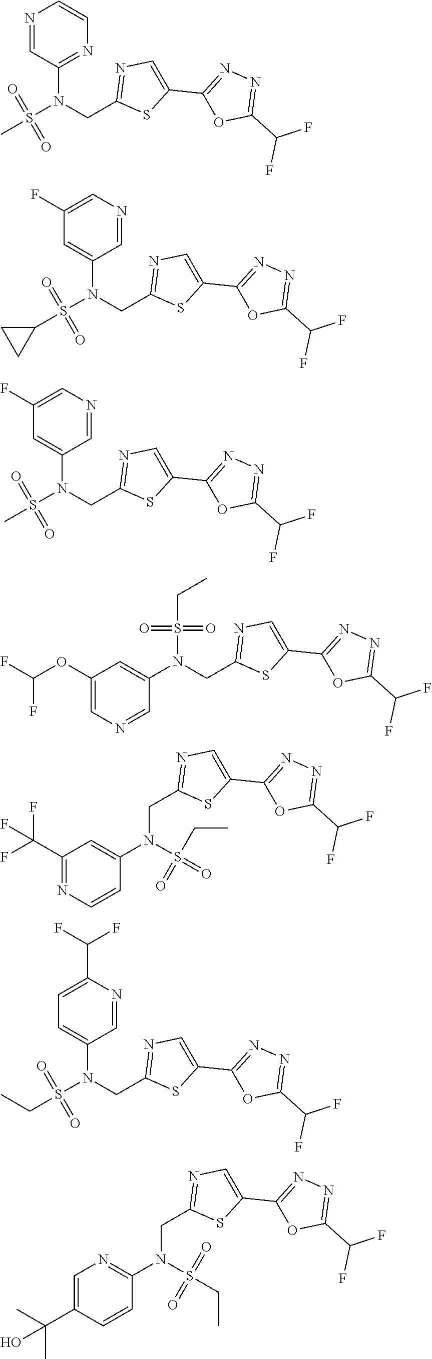

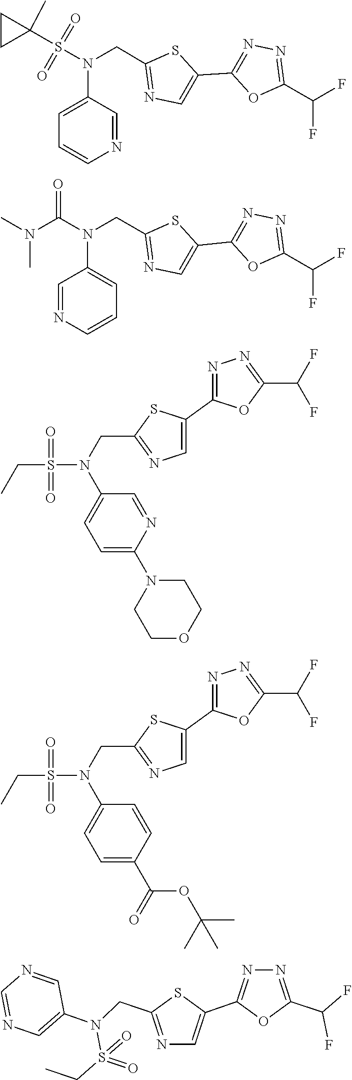

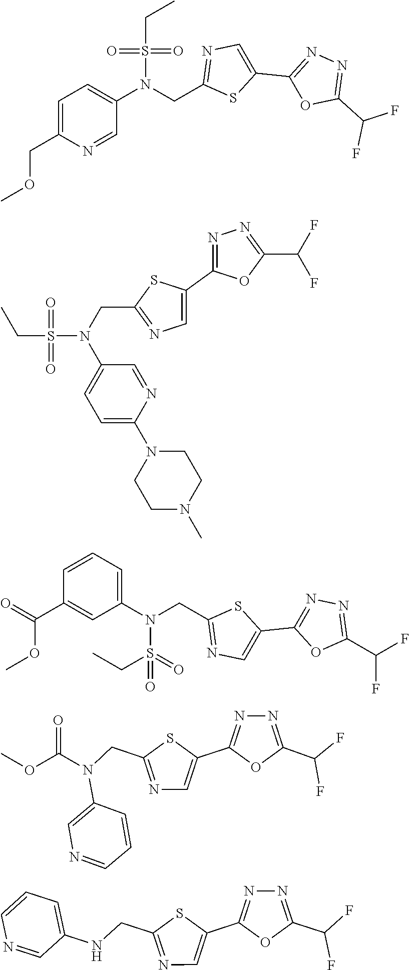 [SMILES: CCS(=O)(=O)N(Cc1ncc(-c2nnc(C(F)F)o2)s1)c1ccc(COC)nc1 ; CCS(=O)(=O)N(Cc1ncc(-c2nnc(C(F)F)o2)s1)c1ccc(N2CCN(C)CC2)nc1 ; CCS(=O)(=O)N(Cc1ncc(-c2nnc(C(F)F)o2)s1)c1cccc(C(=O)OC)c1 ; COC(=O)N(Cc1ncc(-c2nnc(C(F)F)o2)s1)c1cccnc1 ; FC(F)c1nnc(-c2cnc(CNc3cccnc3)s2)o1]

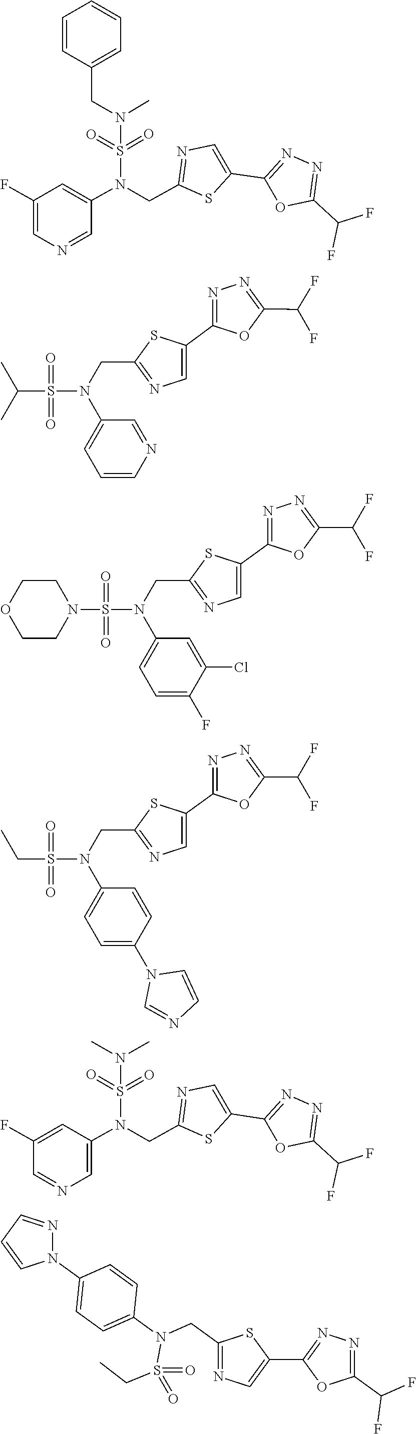

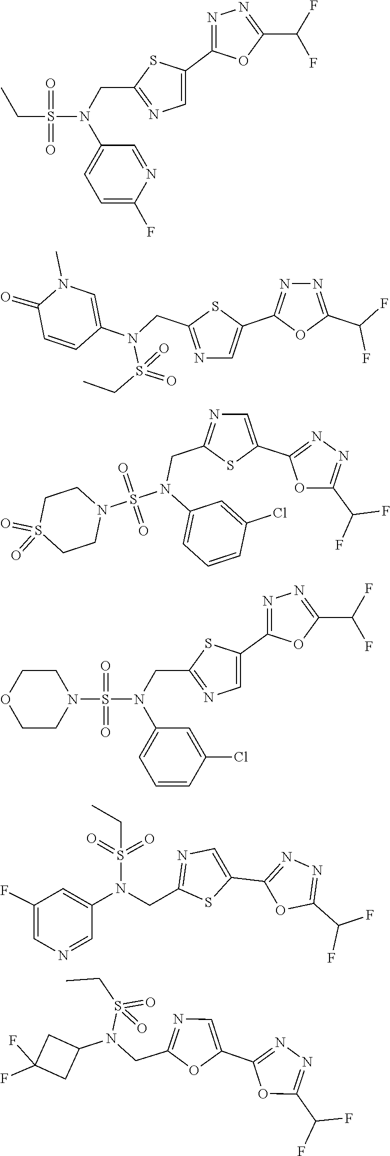

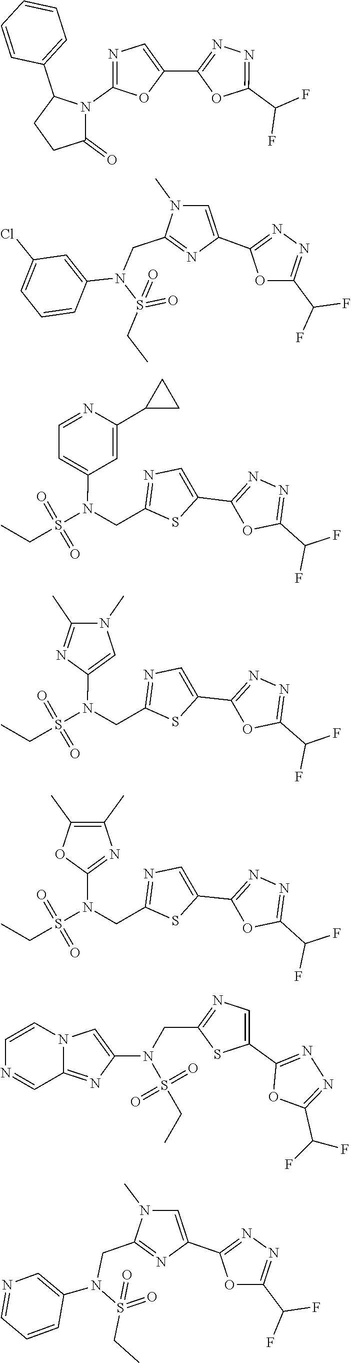

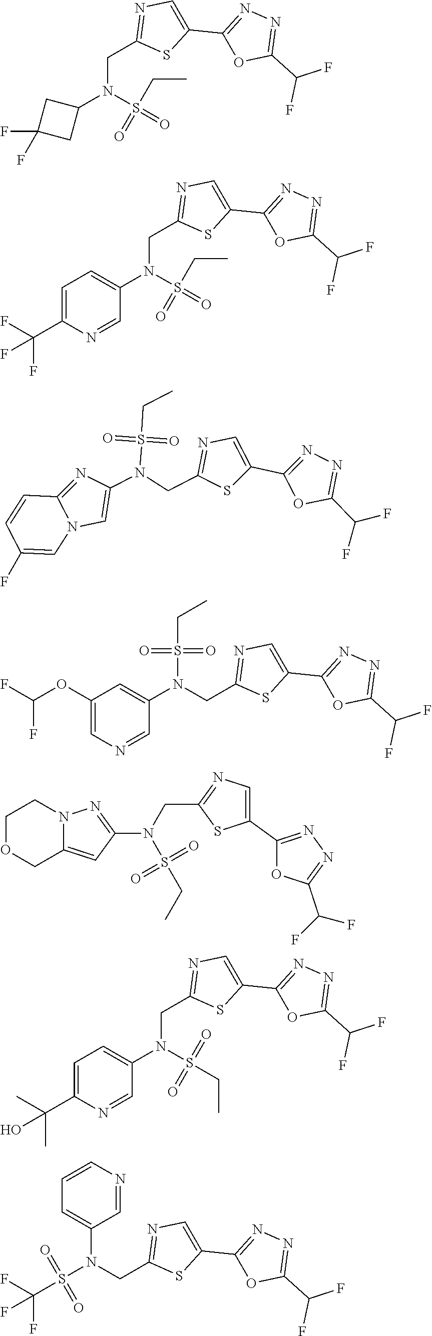

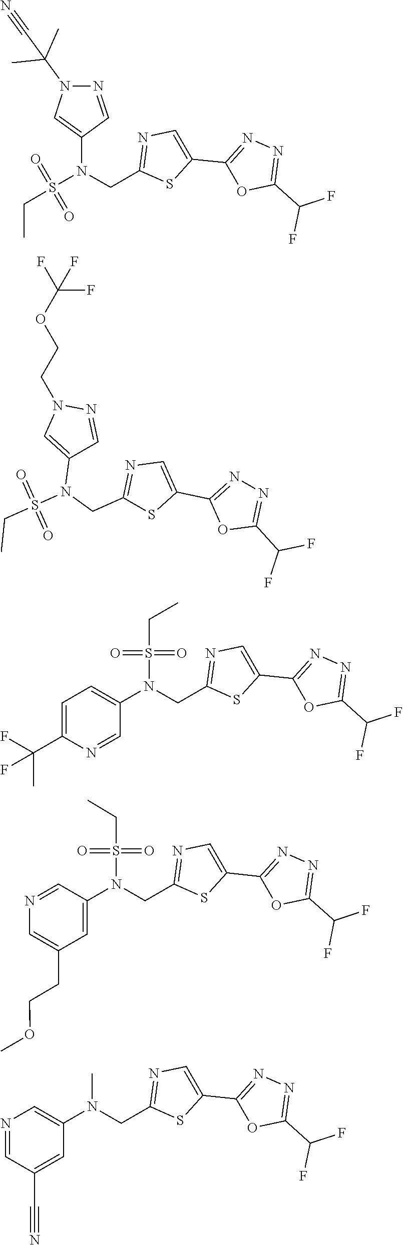

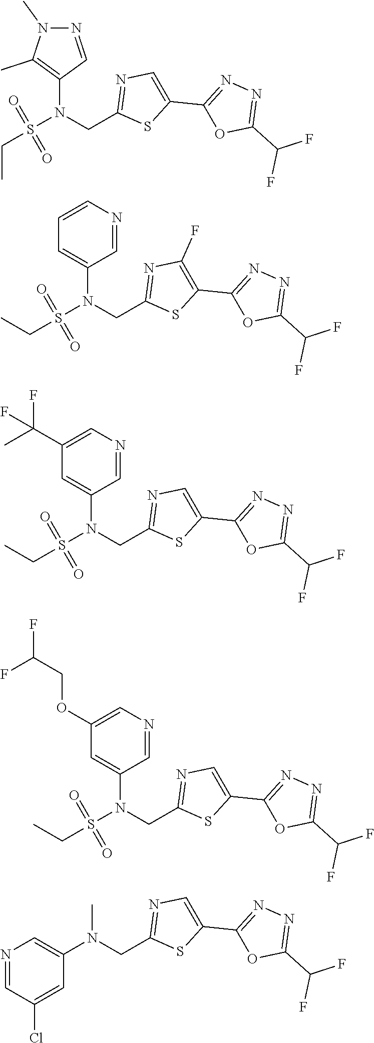 [SMILES: CCS(=O)(=O)N(Cc1ncc(-c2nnc(C(F)F)o2)s1)c1cnn(C)c1C ; CCS(=O)(=O)N(Cc1nc(F)c(-c2nnc(C(F)F)o2)s1)c1cccnc1 ; CCS(=O)(=O)N(Cc1ncc(-c2nnc(C(F)F)o2)s1)c1cncc(C(C)(F)F)c1 ; CCS(=O)(=O)N(Cc1ncc(-c2nnc(C(F)F)o2)s1)c1cncc(OCC(F)F)c1 ; CN(Cc1ncc(-c2nnc(C(F)F)o2)s1)c1cncc(Cl)c1]

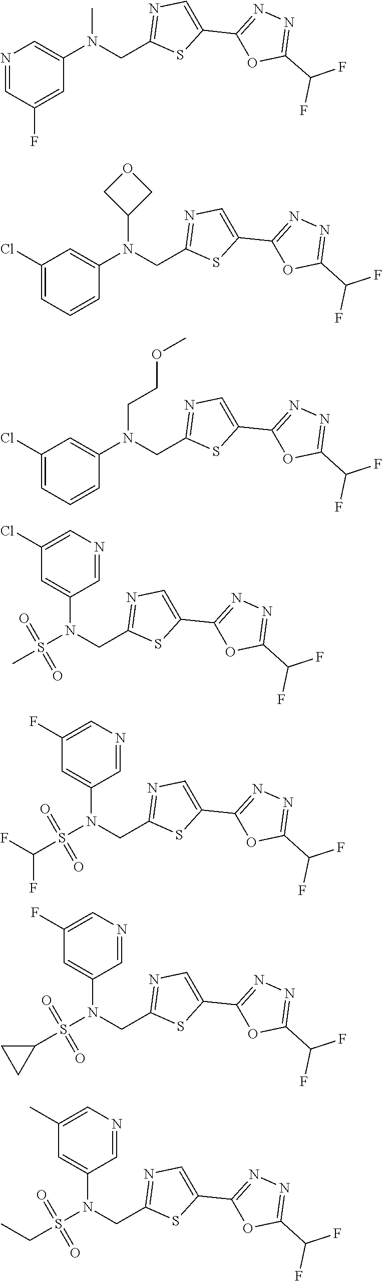

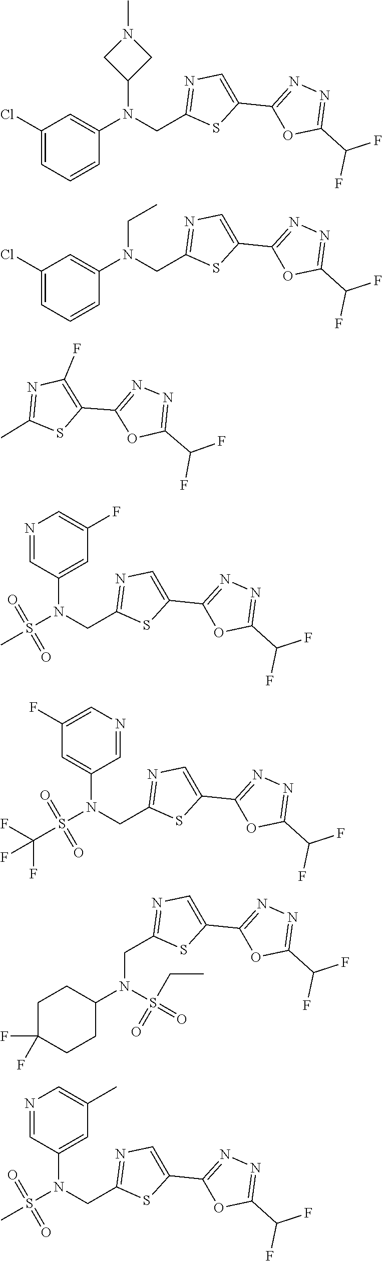

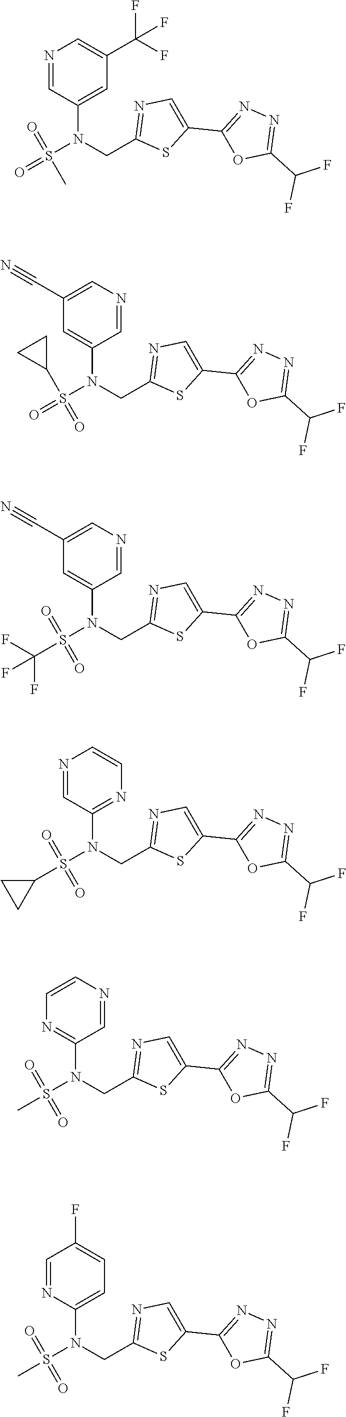 [SMILES: CS(=O)(=O)N(Cc1ncc(-c2nnc(C(F)F)o2)s1)c1cncc(C(F)(F)F)c1 ; N#Cc1cncc(N(Cc2ncc(-c3nnc(C(F)F)o3)s2)S(=O)(=O)C2CC2)c1 ; N#Cc1cncc(N(Cc2ncc(-c3nnc(C(F)F)o3)s2)S(=O)(=O)C(F)(F)F)c1 ; O=S(=O)(C1CC1)N(Cc1ncc(-c2nnc(C(F)F)o2)s1)c1cnccn1 ; CS(=O)(=O)N(Cc1ncc(-c2nnc(C(F)F)o2)s1)c1cnccn1 ; CS(=O)(=O)N(Cc1ncc(-c2nnc(C(F)F)o2)s1)c1ccc(F)cn1]

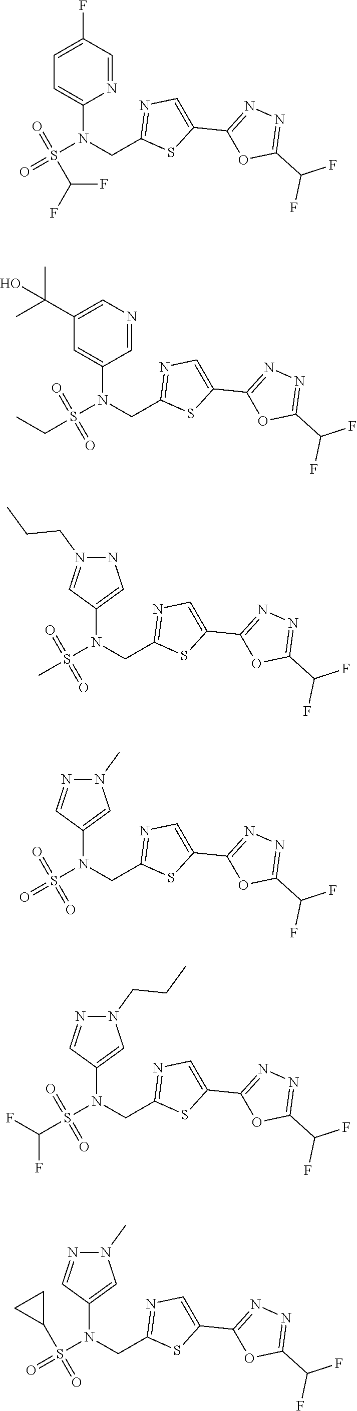

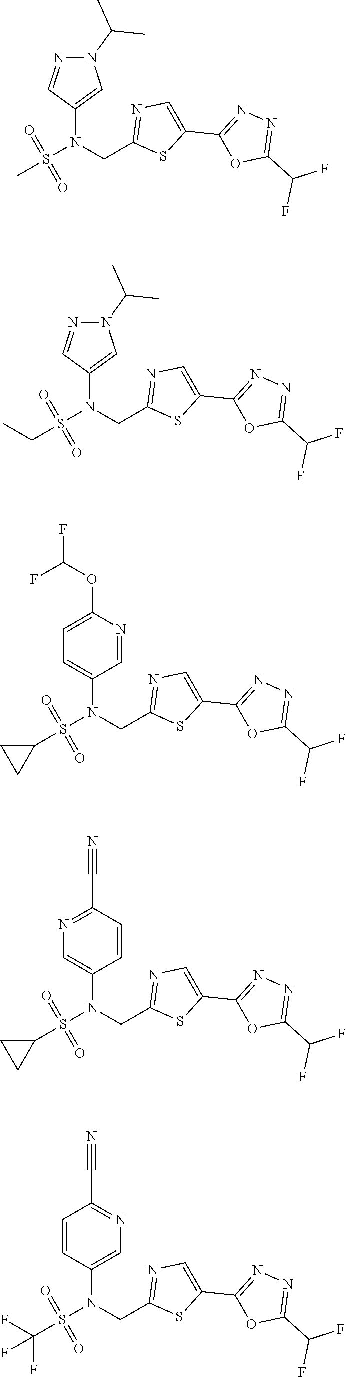 [SMILES: CC(C)n1cc(N(Cc2ncc(-c3nnc(C(F)F)o3)s2)S(C)(=O)=O)cn1 ; CCS(=O)(=O)N(Cc1ncc(-c2nnc(C(F)F)o2)s1)c1cnn(C(C)C)c1 ; O=S(=O)(C1CC1)N(Cc1ncc(-c2nnc(C(F)F)o2)s1)c1ccc(OC(F)F)nc1 ; N#Cc1ccc(N(Cc2ncc(-c3nnc(C(F)F)o3)s2)S(=O)(=O)C2CC2)cn1 ; N#Cc1ccc(N(Cc2ncc(-c3nnc(C(F)F)o3)s2)S(=O)(=O)C(F)(F)F)cn1]

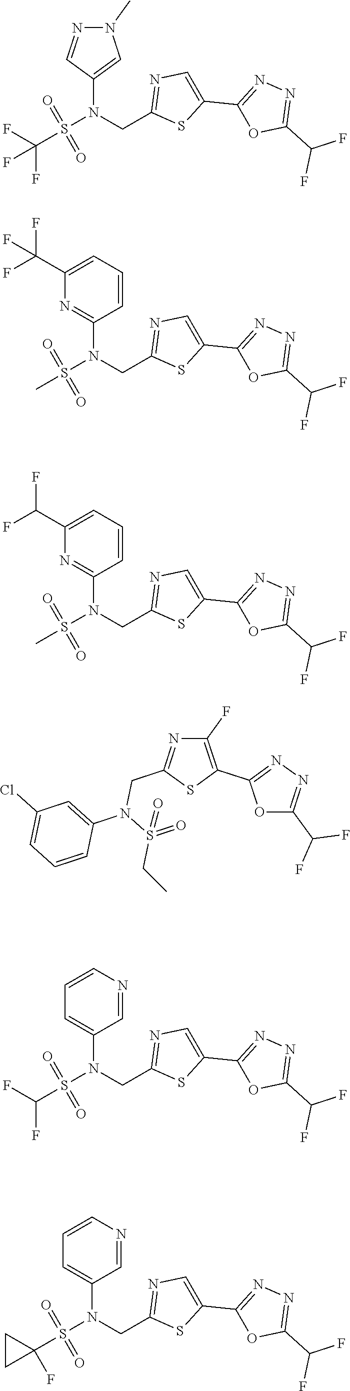

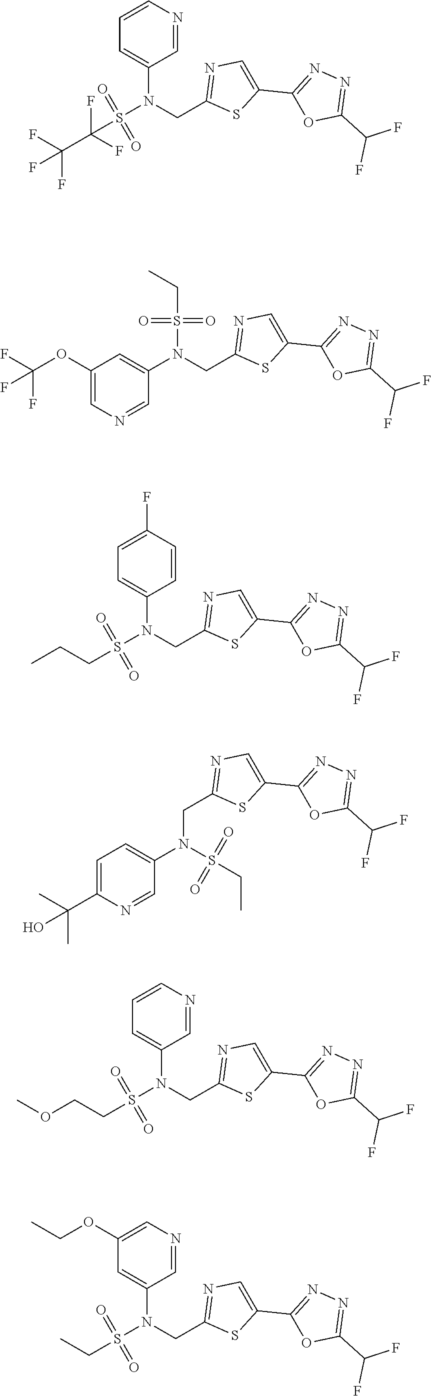 [SMILES: O=S(=O)(N(Cc1ncc(-c2nnc(C(F)F)o2)s1)c1cccnc1)C(F)(F)C(F)(F)F ; CCS(=O)(=O)N(Cc1ncc(-c2nnc(C(F)F)o2)s1)c1cncc(OC(F)(F)F)c1 ; CCCS(=O)(=O)N(Cc1ncc(-c2nnc(C(F)F)o2)s1)c1ccc(F)cc1 ; CCS(=O)(=O)N(Cc1ncc(-c2nnc(C(F)F)o2)s1)c1ccc(C(C)(C)O)nc1 ; COCCS(=O)(=O)N(Cc1ncc(-c2nnc(C(F)F)o2)s1)c1cccnc1 ; CCOc1cncc(N(Cc2ncc(-c3nnc(C(F)F)o3)s2)S(=O)(=O)CC)c1]

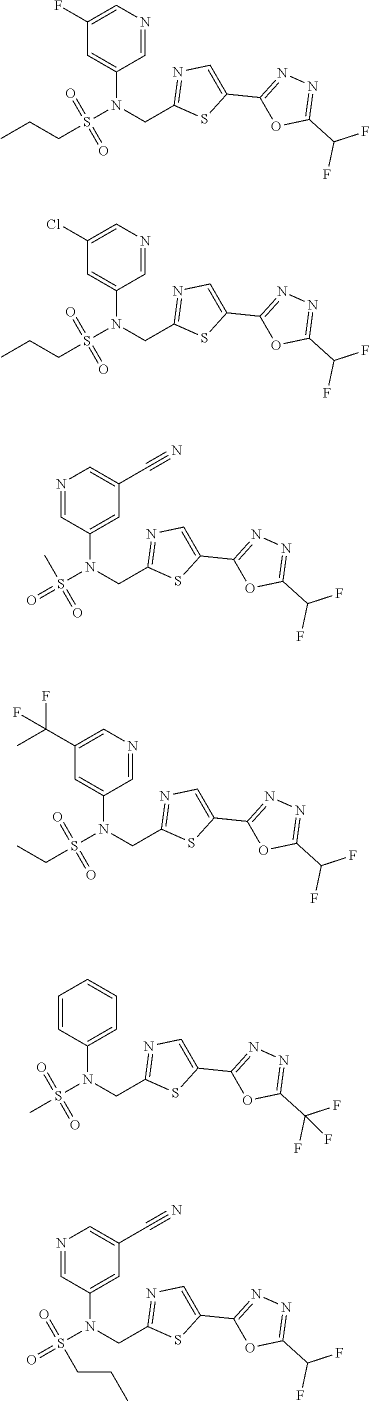 [SMILES: CCCS(=O)(=O)N(Cc1ncc(-c2nnc(C(F)F)o2)s1)c1cncc(F)c1 ; CCCS(=O)(=O)N(Cc1ncc(-c2nnc(C(F)F)o2)s1)c1cncc(Cl)c1 ; CS(=O)(=O)N(Cc1ncc(-c2nnc(C(F)F)o2)s1)c1cncc(C#N)c1 ; CCS(=O)(=O)N(Cc1ncc(-c2nnc(C(F)F)o2)s1)c1cncc(C(C)(F)F)c1 ; CS(=O)(=O)N(Cc1ncc(-c2nnc(C(F)(F)F)o2)s1)c1ccccc1 ; CCCS(=O)(=O)N(Cc1ncc(-c2nnc(C(F)F)o2)s1)c1cncc(C#N)c1]

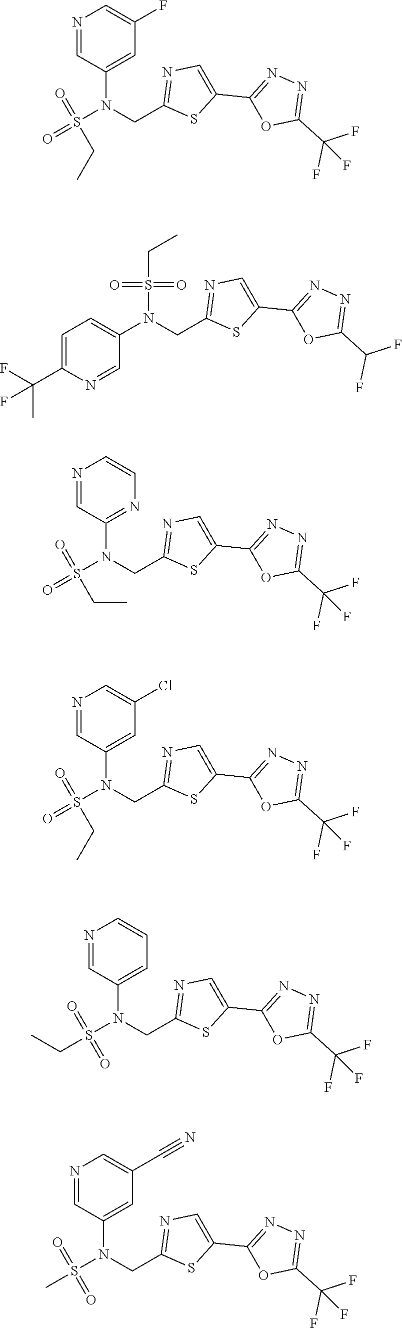 [SMILES: CCS(=O)(=O)N(Cc1ncc(-c2nnc(C(F)(F)F)o2)s1)c1cncc(F)c1 ; CCS(=O)(=O)N(Cc1ncc(-c2nnc(C(F)F)o2)s1)c1ccc(C(C)(F)F)nc1 ; CCS(=O)(=O)N(Cc1ncc(-c2nnc(C(F)(F)F)o2)s1)c1cnccn1 ; CCS(=O)(=O)N(Cc1ncc(-c2nnc(C(F)(F)F)o2)s1)c1cncc(Cl)c1 ; CCS(=O)(=O)N(Cc1ncc(-c2nnc(C(F)(F)F)o2)s1)c1cccnc1 ; CS(=O)(=O)N(Cc1ncc(-c2nnc(C(F)(F)F)o2)s1)c1cncc(C#N)c1]

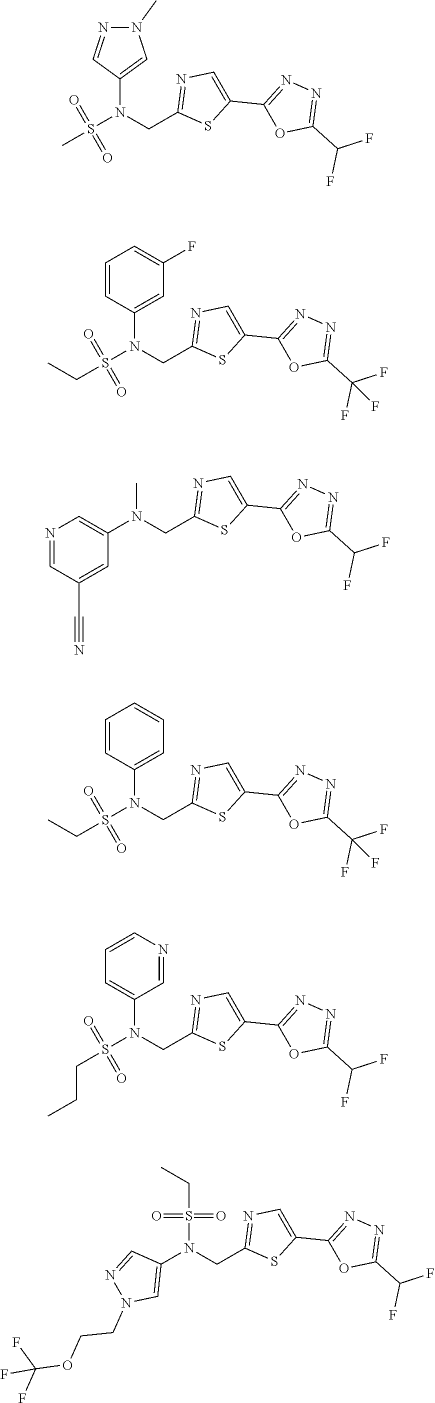 [SMILES: Cn1cc(N(Cc2ncc(-c3nnc(C(F)F)o3)s2)S(C)(=O)=O)cn1 ; CCS(=O)(=O)N(Cc1ncc(-c2nnc(C(F)(F)F)o2)s1)c1cccc(F)c1 ; CN(Cc1ncc(-c2nnc(C(F)F)o2)s1)c1cncc(C#N)c1 ; CCS(=O)(=O)N(Cc1ncc(-c2nnc(C(F)(F)F)o2)s1)c1ccccc1 ; CCCS(=O)(=O)N(Cc1ncc(-c2nnc(C(F)F)o2)s1)c1cccnc1 ; CCS(=O)(=O)N(Cc1ncc(-c2nnc(C(F)F)o2)s1)c1cnn(CCOC(F)(F)F)c1]

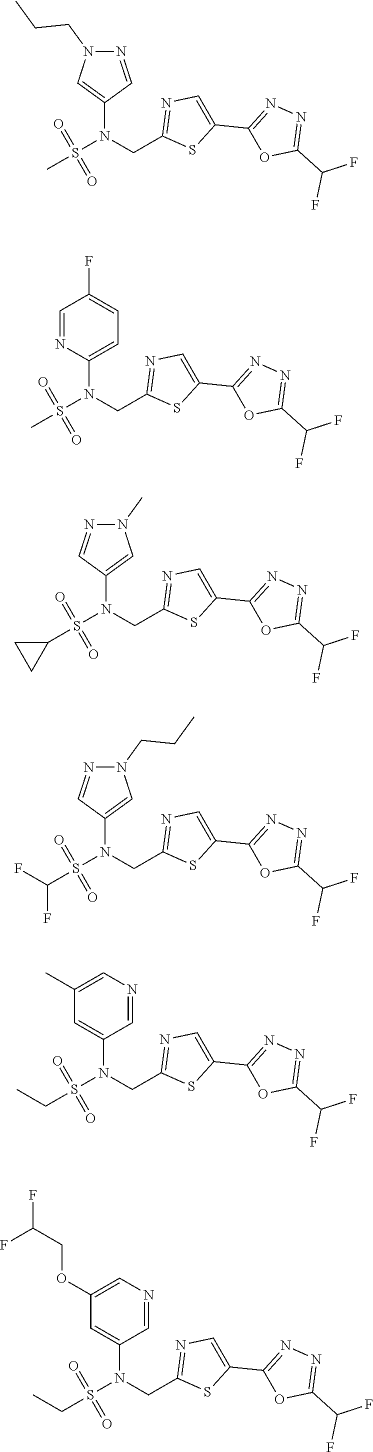 [SMILES: CCCn1cc(N(Cc2ncc(-c3nnc(C(F)F)o3)s2)S(C)(=O)=O)cn1 ; CS(=O)(=O)N(Cc1ncc(-c2nnc(C(F)F)o2)s1)c1ccc(F)cn1 ; Cn1cc(N(Cc2ncc(-c3nnc(C(F)F)o3)s2)S(=O)(=O)C2CC2)cn1 ; CCCn1cc(N(Cc2ncc(-c3nnc(C(F)F)o3)s2)S(=O)(=O)C(F)F)cn1 ; CCS(=O)(=O)N(Cc1ncc(-c2nnc(C(F)F)o2)s1)c1cncc(C)c1 ; CCS(=O)(=O)N(Cc1ncc(-c2nnc(C(F)F)o2)s1)c1cncc(OCC(F)F)c1]

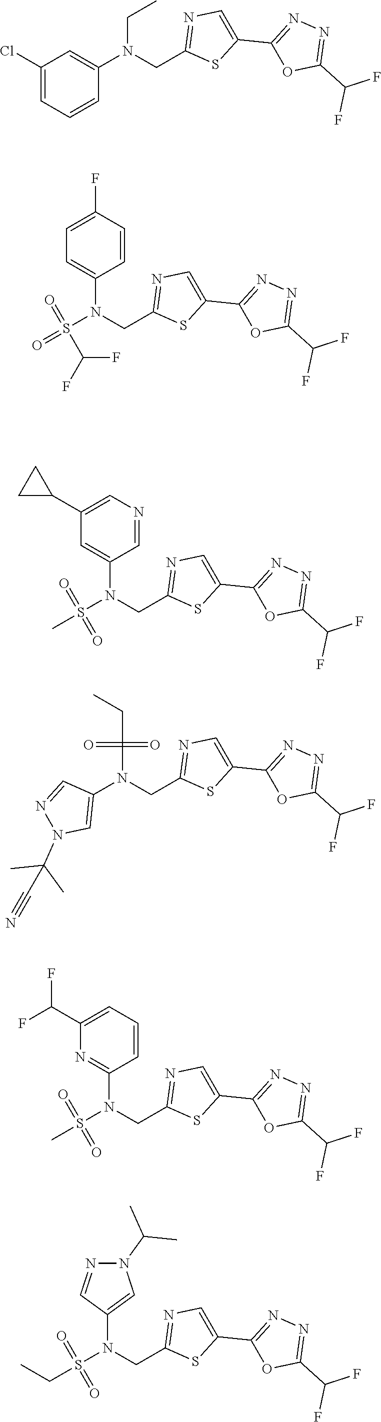

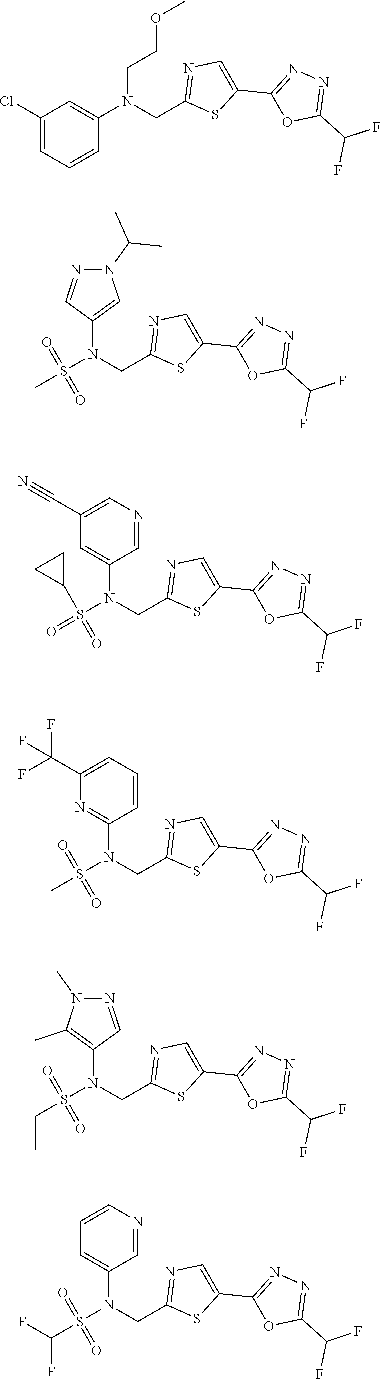 [SMILES: COCCN(Cc1ncc(-c2nnc(C(F)F)o2)s1)c1cccc(Cl)c1 ; CC(C)n1cc(N(Cc2ncc(-c3nnc(C(F)F)o3)s2)S(C)(=O)=O)cn1 ; N#Cc1cncc(N(Cc2ncc(-c3nnc(C(F)F)o3)s2)S(=O)(=O)C2CC2)c1 ; CS(=O)(=O)N(Cc1ncc(-c2nnc(C(F)F)o2)s1)c1cccc(C(F)(F)F)n1 ; CCS(=O)(=O)N(Cc1ncc(-c2nnc(C(F)F)o2)s1)c1cnn(C)c1C ; O=S(=O)(C(F)F)N(Cc1ncc(-c2nnc(C(F)F)o2)s1)c1cccnc1]

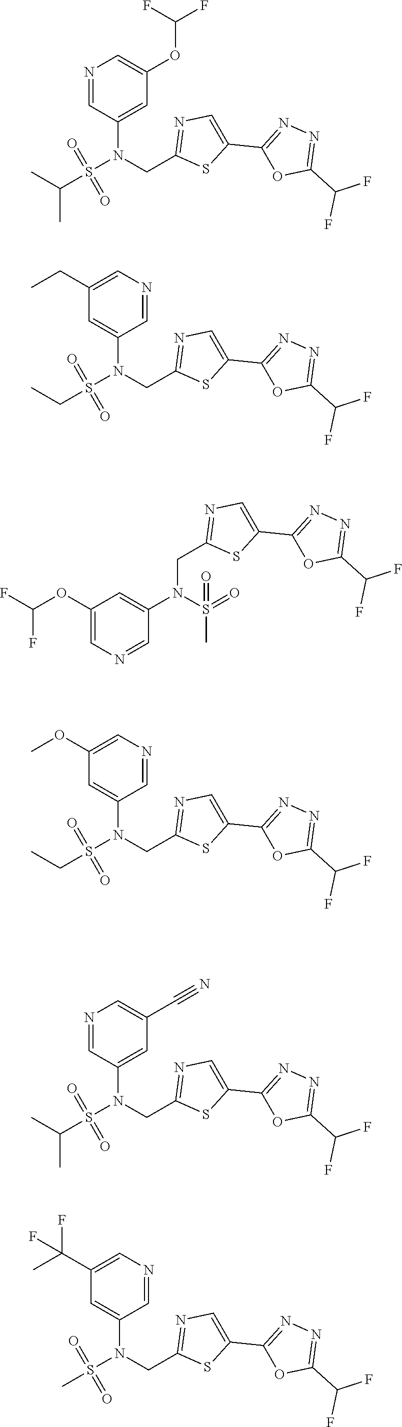 [SMILES: CC(C)S(=O)(=O)N(Cc1ncc(-c2nnc(C(F)F)o2)s1)c1cncc(OC(F)F)c1 ; CCc1cncc(N(Cc2ncc(-c3nnc(C(F)F)o3)s2)S(=O)(=O)CC)c1 ; CS(=O)(=O)N(Cc1ncc(-c2nnc(C(F)F)o2)s1)c1cncc(OC(F)F)c1 ; CCS(=O)(=O)N(Cc1ncc(-c2nnc(C(F)F)o2)s1)c1cncc(OC)c1 ; CC(C)S(=O)(=O)N(Cc1ncc(-c2nnc(C(F)F)o2)s1)c1cncc(C#N)c1 ; CC(F)(F)c1cncc(N(Cc2ncc(-c3nnc(C(F)F)o3)s2)S(C)(=O)=O)c1]

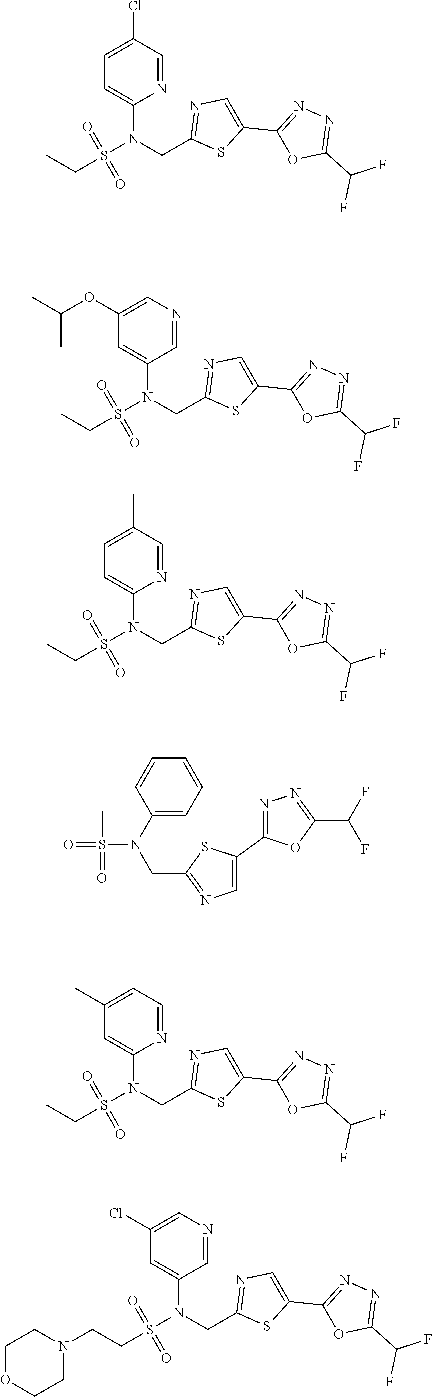 [SMILES: CCS(=O)(=O)N(Cc1ncc(-c2nnc(C(F)F)o2)s1)c1ccc(Cl)cn1 ; CCS(=O)(=O)N(Cc1ncc(-c2nnc(C(F)F)o2)s1)c1cncc(OC(C)C)c1 ; CCS(=O)(=O)N(Cc1ncc(-c2nnc(C(F)F)o2)s1)c1ccc(C)cn1 ; CS(=O)(=O)N(Cc1ncc(-c2nnc(C(F)F)o2)s1)c1ccccc1 ; CCS(=O)(=O)N(Cc1ncc(-c2nnc(C(F)F)o2)s1)c1cc(C)ccn1 ; O=S(=O)(CCN1CCOCC1)N(Cc1ncc(-c2nnc(C(F)F)o2)s1)c1cncc(Cl)c1]

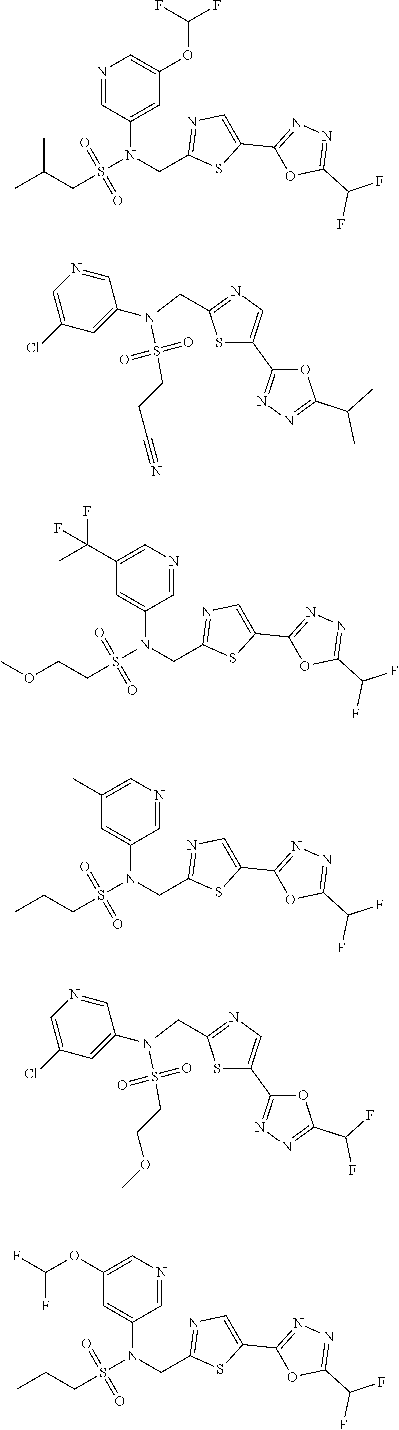

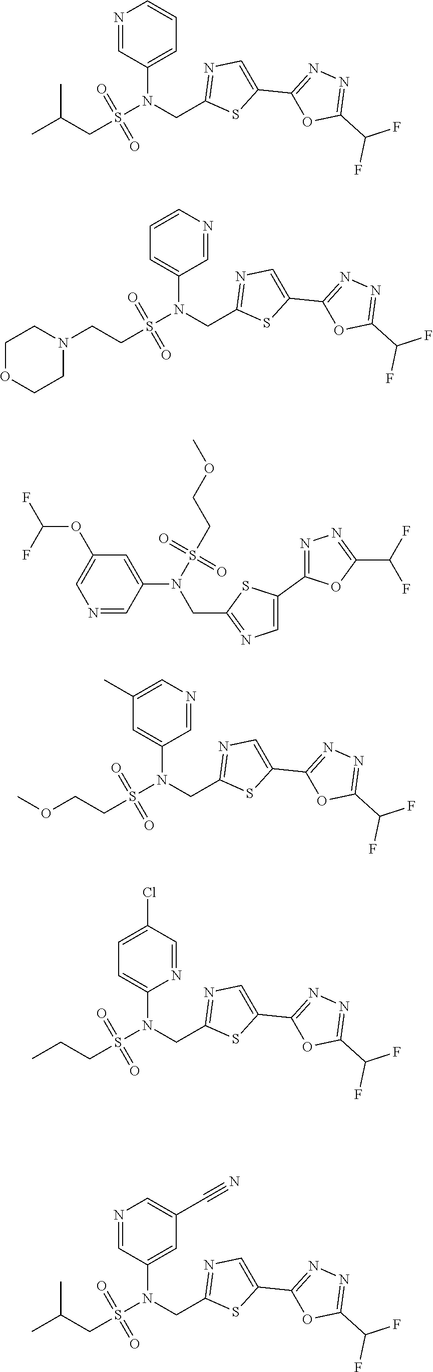 [SMILES: CC(C)CS(=O)(=O)N(Cc1ncc(-c2nnc(C(F)F)o2)s1)c1cccnc1 ; O=S(=O)(CCN1CCOCC1)N(Cc1ncc(-c2nnc(C(F)F)o2)s1)c1cccnc1 ; COCCS(=O)(=O)N(Cc1ncc(-c2nnc(C(F)F)o2)s1)c1cncc(OC(F)F)c1 ; COCCS(=O)(=O)N(Cc1ncc(-c2nnc(C(F)F)o2)s1)c1cncc(C)c1 ; CCCS(=O)(=O)N(Cc1ncc(-c2nnc(C(F)F)o2)s1)c1ccc(Cl)cn1 ; CC(C)CS(=O)(=O)N(Cc1ncc(-c2nnc(C(F)F)o2)s1)c1cncc(C#N)c1]

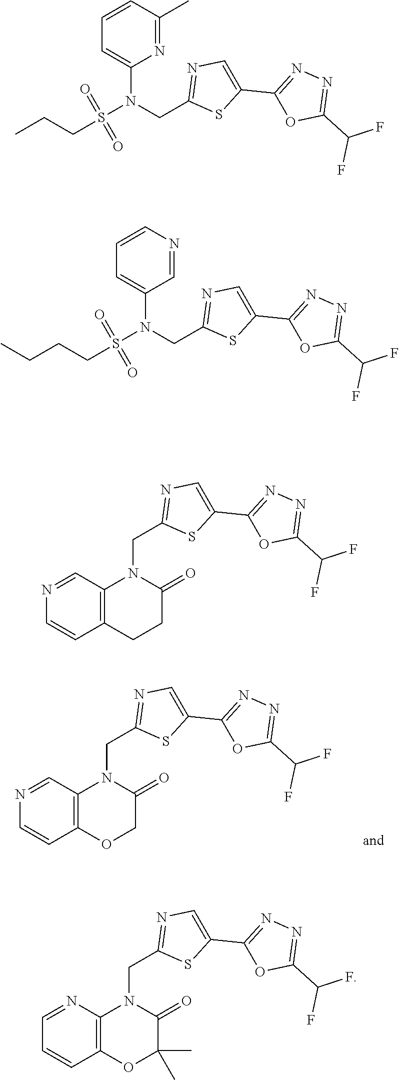 [SMILES: CCCS(=O)(=O)N(Cc1ncc(-c2nnc(C(F)F)o2)s1)c1cccc(C)n1 ; CCCCS(=O)(=O)N(Cc1ncc(-c2nnc(C(F)F)o2)s1)c1cccnc1 ; O=C1CCc2ccncc2N1Cc1ncc(-c2nnc(C(F)F)o2)s1 ; O=C1COc2ccncc2N1Cc1ncc(-c2nnc(C(F)F)o2)s1 ; CC1(C)Oc2cccnc2N(Cc2ncc(-c3nnc(C(F)F)o3)s2)C1=O]

In some embodiments, the present disclosure provides a compound of Formula (Ic) or a pharmaceutically acceptable salt thereof:

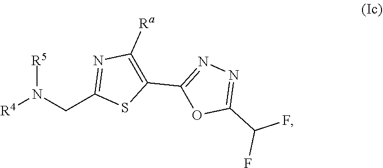

wherein:
R^{a} is H, Me, or F; and
R⁴ and R⁵ are as defined above in Formula (I).

In some embodiments of Formula (Ic), R^{a} is H. In some embodiments, R^{a} is F. In some embodiments, R^{a} is Me.

In some embodiments of Formula (Ic), R⁴ is selected from the group consisting of alkylenealkoxy, alkyleneheterocyclyl, S(O)₂ alkyl, —S(O)₂ cycloaklyl, —S(O)₂ alkylenecycloalkyl, —S(O)₂ alkyleneheterocyclyl, —S(O)₂N(H)alkyleneheterocyclyl, —C(O)alkyl, —C(O)cycloalkyl, —C(O)alkylenecycloalkyl, —C(O)alkyleneheterocyclyl, and —C(O)N(H)alkyleneheterocyclyl. In some embodiments, R⁴ is selected from the group consisting of alkyleneheterocyclyl, —S(O)₂ alkyl, —S(O)₂ cycloalkyl, —S(O)₂ alkyleneheterocyclyl, —C(O)alkyleneheterocyclyl, and —C(O)N(H)alkyleneheterocyclyl. In some embodiments, R⁴ is selected from the group consisting of —S(O)₂ alkyl, —S(O)₂ cycloalkyl, and —S(O)₂ alkyleneheterocyclyl. In some embodiments, R⁴ is —S(O)₂ alkyl. In some embodiments, R⁴ is —S(O)₂ cycloalkyl. In some embodiments, R⁴ is —S(O)₂N(H)alkyleneheterocyclyl. In some embodiments, the alkylene is a C₁₋₅ alkylene and the heterocyclyl is an optionally substituted 4- to 10-membered heterocyclyl having 1, 2, or 3 heteroatoms selected from the group consisting of N, O, and S. In some embodiments, the alkylene is a C₁₋₅ alkylene and the heterocyclyl is an optionally substituted 4- to 7-membered heterocyclyl having 1, 2, or 3 heteroatoms selected from the group consisting of N, O, and S. In some embodiments, the alkylene is a C₂₋₄ alkylene and the heterocyclyl is an optionally substituted 6-membered heterocyclyl having 1, 2, or 3 heteroatoms selected from the group consisting of N, O, and S. In some embodiments, the heterocyclyl is selected from the group consisting of piperidine, morpholine, thiomorpholine, thiomorpholine 1-oxide, thiomorpholine 1,1-dioxide, and piperazine, each of which is optionally substituted. In some embodiments, the optional substituent is selected from the group consisting of alkyl, haloalkyl, alkoxy, acyl, sulfonyl, heteroaryl, and heterocyclyl.

In some embodiments of Formula (Ic), R⁵ is selected from the group consisting of:

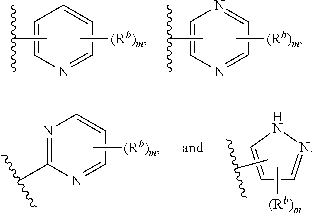

In some embodiments, R⁵ is

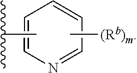

In some embodiments, R⁵ is

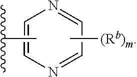

In some embodiments, R⁵ is

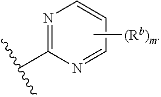

In some embodiments, R⁵ is

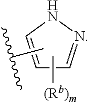

In some embodiments, R^{b} is selected from the group consisting of halogen, haloalkyl, alkyl, Oalkyl, Ohaloalkyl, alkylene-Ohaloalkyl, cycloalkyl, heterocyclyl aryl, heteroaryl, alkylnitrile, or CN. In some embodiments, R^{b} is selected from the group consisting of halo, alkyl, haloalkyl, alkoxy, haloalkoxy, acyl, sulfonyl, cycloalkyl, heteroaryl, and heterocyclyl. In some embodiments, the haloalkyl is selected from CF₃, CF₂CH₃, CHF₂, or CH₂F. In some embodiments, the alkyl is a —C₁₋₅ alkyl. In some embodiments, —C₁₋₅ alkyl is methyl, ethyl, propyl, i-propyl, butyl, or t-butyl. In some embodiments, methyl, ethyl, propyl, i-propyl, butyl, or t-butyl is optionally substituted with OH. In some embodiments, the cycloalkyl is a C₃₋₆ cycloalkyl. In some embodiments, the aryl is a phenyl. In some embodiments, the heteroaryl is 5- or 6-membered heteroaryl having 1, 2, or 3 heteroatoms selected from N, O, and S. In some embodiments, the heterocyclyl is a 4- to 7-member heterocyclyl with 1 or 2 heteroatoms selected from N, O, and S. In some embodiments, the Ohaloalkyl is selected from OCF₃, OCHF₂, or OCH₂F. In some embodiments, the Oalkyl is O-methyl, O-ethyl, O-propyl, O-i-propyl, O-butyl, or O-t-butyl. In some embodiments, R^{b} is selected from the group consisting of F, Cl, —CH₃, —CH₂CH₃, —CF₃, —CHF₂, —CF₂CH₃, —CN, —OCH₃, —OCH₂CH₃, —OCH(CH₃)₂, —OCHF₂, —OCH₂CF₂H, and cyclopropyl. In some embodiments, m is 0, 1, or 2. In some embodiments, m is 0 or 1. In some embodiments, m is 0. In some embodiments, m is 1. In some embodiments, m is 2.

In some embodiments, the present disclosure provides a compound of Formula (Id) or a pharmaceutically acceptable salt thereof:

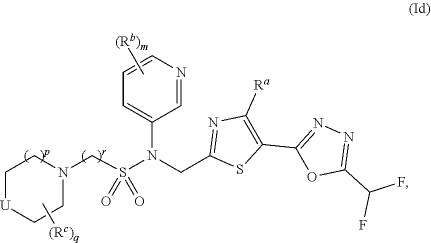

wherein:
U is NR^{d}, O, S, S(O), S(O)₂, CH₂, CHF, or CF₂;
R^{a} is H, Me, or F;
R^{b} is each independently halo, alkyl, haloalkyl, alkoxy, haloalkoxy, —C(O)R^{e}, —C(O)OR^{e}, —C(O)N(R^{e})(R^{e′}), —S(O₂)R^{e}, cycloalkyl, heteroaryl, or heterocyclyl;
R^{c} is each independently F, alkyl, haloalkyl, alkoxy, haloalkoxy, —C(O)R^{e}, —C(O)OR^{e}, —C(O)N(R^{e})(R^{e′}), —S(O₂)R^{e}, heteroaryl, or heterocyclyl, and/or two R^{c} groups taken together with the carbon atoms to which they are attached form a bridged or fused C₃₋₇ cycloalkyl, a bridged or fused 4- to 7-membered heterocyclyl; or a 5- or 6-membered heteroaryl, each of which is optionally substituted;
R^{d} is H, alkyl, acyl, sulfonyl, cycloalkyl, aryl, or heteroaryl;
R^{e} and R^{e′} is each independently H, alkyl, cycloalkyl, heterocyclyl, aryl, heteroaryl, —CH₂ cycloalkyl, —CH₂ heterocyclyl, —CH₂ aryl, or —CH₂ heteroaryl;
m is 0, 1, 2, or 3;
p is 0, 1, 2, or 3;
q is 0, 1, or 2; and
r is 1, 2, 3, or 4.

In some embodiments, the present disclosure provides a compound of Formula (Ie) or a pharmaceutically acceptable salt thereof:

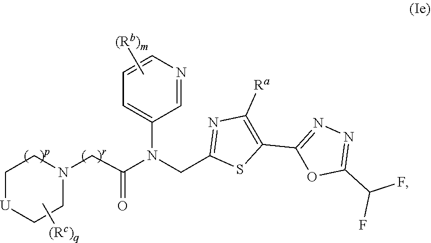

wherein:
U is NR^{d}, O, S, S(O), S(O)₂, CH₂, CHF, or CF₂;
R^{a} is H, Me, or F;
R^{b} is each independently halo, alkyl, haloalkyl, alkoxy, haloalkoxy, —C(O)R^{e}, —C(O)OR^{e}, —C(O)N(R^{e})(R^{e′}), sulfonyl, cycloalkyl, heteroaryl, or heterocyclyl; R^{c} is each independently F, alkyl, haloalkyl, alkoxy, haloalkoxy, —C(O)R^{e}, —C(O)OR^{e}, —C(O)N(R^{e})(R^{e′}), —S(O₂)R^{e}, heteroaryl, or heterocyclyl, and/or two R^{c} groups taken together with the carbon atoms to which they are attached form a bridged or fused C₃₋₇ cycloalkyl, a bridged or fused 4- to 6-membered heterocyclyl; or a 5- or 6-membered heteroaryl, each of which is optionally substituted;
R^{d} is H, alkyl, acyl, sulfonyl, cycloalkyl, aryl, or heteroaryl;
R^{e} and R^{e′} is each independently H, alkyl, cycloalkyl, heterocyclyl, aryl, heteroaryl, —CH₂ cycloalkyl, —CH₂ heterocyclyl, —CH₂ aryl, or —CH₂ heteroaryl;
m is 0, 1, 2, or 3;
p is 0, 1, 2, or 3;
q is 0, 1, or 2; and
r is 1, 2, 3, or 4.

In some embodiments, the present disclosure provides a compound of Formula (If) or a pharmaceutically acceptable salt thereof:

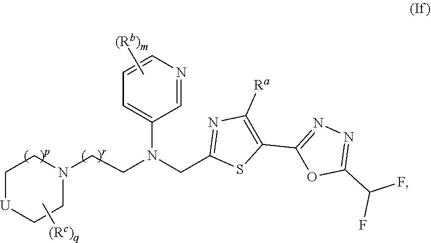

wherein:
U is NR^{d}, O, S, S(O), S(O)₂, CH₂, CHF, or CF₂;
R^{a} is H, Me, or F;
R^{b} is each independently halo, alkyl, haloalkyl, alkoxy, haloalkoxy, —C(O)R^{e}, —C(O)OR^{e}, —C(O)N(R^{e})(R^{e′}), sulfonyl, cycloalkyl, heteroaryl, or heterocyclyl;
R^{c} is each independently F, alkyl, haloalkyl, alkoxy, haloalkoxy, —C(O)R^{e}, —C(O)OR^{e}, —C(O)N(R^{e})(R^{e′}), —S(O₂)R^{e}, heteroaryl, or heterocyclyl, and/or two R^{c} groups taken together with the carbon atoms to which they are attached form a bridged or fused C₃₋₇ cycloalkyl, a bridged or fused 4- to 7-membered heterocyclyl; or a 5- or 6-membered heteroaryl, each of which is optionally substituted;
R^{d} is H, alkyl, acyl, sulfonyl, cycloalkyl, aryl, or heteroaryl;
R^{e} and R^{e′} is each independently H, alkyl, cycloalkyl, heterocyclyl, aryl, heteroaryl, —CH₂ cycloalkyl, —CH₂ heterocyclyl, —CH₂ aryl, or —CH₂ heteroaryl;
m is 0, 1, 2, or 3;
p is 0, 1, 2, or 3;
q is 0, 1, or 2; and
r is 1, 2, 3, or 4.

In some embodiments, the present disclosure provides a compound of Formula (Ig) or a pharmaceutically acceptable salt thereof:

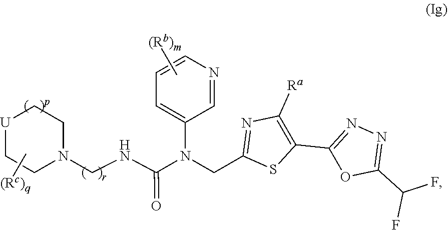

wherein:
U is NR^{d}, O, S, S(O), S(O)₂, CH₂, CHF, or CF₂;
R^{a} is H, Me, or F;
R^{b} is each independently halo, alkyl, haloalkyl, alkoxy, haloalkoxy, —C(O)R^{e}, —C(O)OR^{e}, —C(O)N(R^{e})(R^{e′}), sulfonyl, cycloalkyl, heteroaryl, or heterocyclyl;
R^{c} is each independently F, alkyl, haloalkyl, alkoxy, haloalkoxy, —C(O)R^{e}, —C(O)OR^{e}, —C(O)N(R^{e})(R^{e′}), —S(O₂)R^{e}, heteroaryl, or heterocyclyl, and/or two R^{c} groups taken together with the carbon atoms to which they are attached form a bridged or fused C₃₋₇ cycloalkyl, a bridged or fused 4- to 7-membered heterocyclyl; or a 5- or 6-membered heteroaryl, each of which is optionally substituted;
R^{d} is H, alkyl, acyl, sulfonyl, cycloalkyl, aryl, or heteroaryl;
R^{e} and R^{e′} is each independently H, alkyl, cycloalkyl, heterocyclyl, aryl, heteroaryl, —CH₂ cycloalkyl, —CH₂ heterocyclyl, —CH₂ aryl, or —CH₂ heteroaryl;
m is 0, 1, 2, or 3;
p is 0, 1, 2, or 3;
q is 0, 1, or 2; and
r is 1, 2, 3, or 4.

In some embodiments, the compound has the formula:

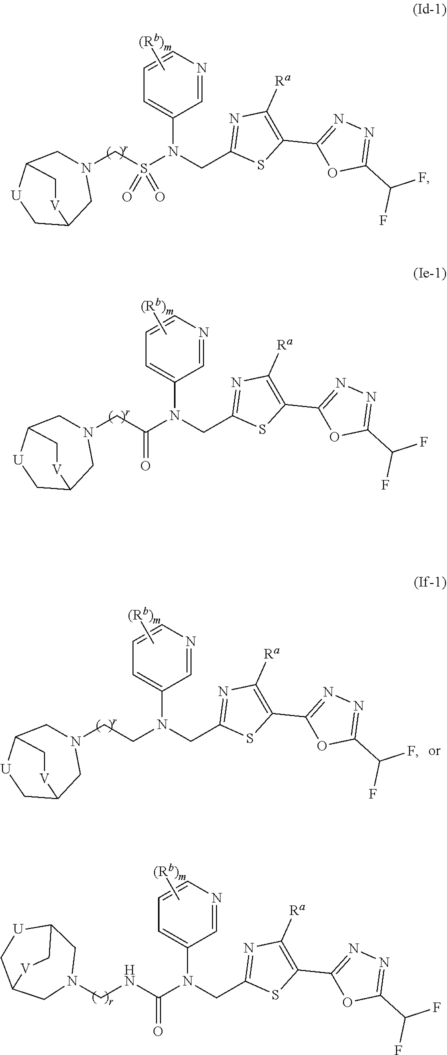

(Ig-1), wherein:
U, R^{a}, R^{b}, m, and r are as defined above in Formulas (Id), (le), (If), and (Ig); and
V is O or NR^{d}.

In some embodiments of Formulas (Id)-(Ig) and (Id-1)-(Ig-1), U is NR^{d}, O, or S and V is O. In some embodiments, U is N, O, or S and V is NR^{d}. In some embodiments, U is NR^{d} and V is NR^{d}. In some embodiments, U is O and V is NR^{d}. In some embodiments, U is S and V is NR^{d}. In some embodiments, U is NR^{d} and V is O. In some embodiments, U is O and V is O. In some embodiments, U is S and V is O.

In some embodiments of Formulas (Id)-(Ig) and (Id-1)-(Ig-1), U is O, S, S(O)₂, CH₂, or NR^{d}. In some embodiments, U is O, S, CH₂, or NR^{d}. In some embodiments, U is O, S, or NR^{d}. In some embodiments, U is O or CH₂. In some embodiments, U is O. In some embodiments, U is S. In some embodiments, U is NR^{d}. In some embodiments, U is S(O)₂.

In some embodiments of Formulas (Id)-(Ig) and (Id-1)-(Ig-1), R^{a} is H. In some embodiments, R^{a} is F. In some embodiments, R^{a} is Me.

In some embodiments of Formulas (Id)-(Ig) and (Id-1)-(Ig-1), R^{b} is halo, alkyl, haloalkyl, alkyl, haloalkoxy, cycloalkyl, heterocyclyl, heteroaryl, or nitrile. In some embodiments, R^{b} is halo, alkyl, haloalkyl, alkyl, haloalkoxy, cycloalkyl, or nitrile. In some embodiments, the haloalkyl is selected from CF₃, CF₂CH₃, CHF₂, or CH₂F. In some embodiments, the alkyl is a —C₁₋₅ alkyl. In some embodiments, —C₁₋₅ alkyl is methyl, ethyl, propyl, i-propyl, butyl, or t-butyl. In some embodiments, the cycloalkyl is a C₃₋₆ cycloalkyl. In some embodiments, the heteroaryl is 5- or 6-membered heteroaryl having 1, 2, or 3 heteroatoms selected from N, O, and S. In some embodiments, the heterocyclyl is a 4- to 7-member heterocyclyl with 1 or 2 heteroatoms selected from N, O, and S. In some embodiments, the haloalkoxy is selected from OCF₃, OCHF₂, or OCH₂F. In some embodiments, the alkoxy is O-methyl, O-ethyl, O-propyl, O-i-propyl, O-butyl, or O-t-butyl. In some embodiments, R^{b} is —C(O)R^{e}, —C(O)OR^{e}, —C(O)N(R^{e})(R^{e′}).

In some embodiments of Formulas (Id)-(Ig), R^{c} is F, C₁₋₅ alkyl, haloalkyl, C₁₋₅ alkoxy, haloalkoxy, acyl, sulfonyl, 5- or 6-membered heteroaryl, or C₃₋₆ heterocyclyl. In some embodiments, R^{c} is —C(O)R^{e}, —C(O)OR^{e}, —C(O)N(R^{e})(R^{e′}). In some embodiments, two R^{c} groups taken together with the carbon atoms to which they are attached form a bridged or fused C₃₋₇ cycloalkyl, a bridged or fused 5- or 6-membered heterocyclyl, or a 5- or 6-membered heteroaryl, each of which is optionally substituted. In some embodiments, two R^{c} groups taken together with the carbon atoms to which they are attached form an optionally substituted bridged or fused C₃₋₇ cycloalkyl. In some embodiments, two R^{c} groups taken together with the carbon atoms to which they are attached form an optionally substituted bridged or fused 5- or 6-membered heterocyclyl. In some embodiments, two R^{c} groups taken together with the carbon atoms to which they are attached form an alkoxy or aminoalkyl bridge. In some embodiments, the optional substituent is one or more R^{b}, as defined above. In some embodiments, the optional substituent is selected from the group consisting of F, C₁₋₅ alkyl, C₁₋₅ alkoxy, CF₃, CF₂H, CFH₂, —OCF₃, —OCF₂H, —OCFH₂, —C(O)R^{e}, —C(O)OR^{e}, —C(O)N(R^{e})(R^{e′}), and —SO₂R^{e}. In some embodiments, the optional substituent is selected from the group consisting of F, C₁₋₅ alkyl, C₁₋₅ alkoxy, CF₃, CF₂H, CFH₂, —OCF₃, —OCF₂H, and —OCFH₂. In some embodiments, the optional substituent is F or C₁₋₅ alkyl. In some embodiments, the optional substituent is F. In some embodiments, the optional substituent is C₁₋₅ alkyl. In some embodiments, the C₁₋₅ alkyl is methyl. In some embodiments, the C₁₋₅ alkyl is ethyl. In some embodiments, the C₁₋₅ alkyl is propyl. In some embodiments, the C₁₋₅ alkyl is isopropyl.

In some embodiments of Formulas (Id)-(Ig) and (Id-1)-(Ig-1), R^{e} and R^{e′} is each independently H, alkyl, cycloalkyl, or —CH₂ cycloalkyl. In some embodiments, the alkyl is a —C₁₋₅ alkyl. In some embodiments, —C₁₋₅ alkyl is methyl, ethyl, propyl, i-propyl, butyl, or t-butyl. In some embodiments, the cycloalkyl is a C₃₋₆ cycloalkyl. In some embodiments, the cycloalkyl is cyclopropyl. In some embodiments, R^{e} and R^{e′} are H.

In some embodiments of Formulas (Id)-(Ig) and (Id-1)-(Ig-1), m is 0, 1, or 2. In some embodiments, m is 0 or 1. In some embodiments, m is 0. In some embodiments, m is 1. In some embodiments, m is 2.

In some embodiments of Formulas (Id)-(Ig), p is 0, 1, or 2. In some embodiments, p is 0 or 1. In some embodiments, p is 1 or 2. In some embodiments, p is 0. In some embodiments, p is 1. In some embodiments, p is 2.

In some embodiments of Formulas (Id)-(Ig) and (Id-1)-(Ig-1), r is 1, 2, or 3. In some embodiments, r is 1 or 2. In some embodiments, r is 2 or 3. In some embodiments, r is 1. In some embodiments, r is 2. In some embodiments, r is 3. In some embodiments, r is 4.

In some embodiments of Formulas (Id)-(Ig), q is 0 or 1. In some embodiments, q is 0. In some embodiments, q is 1. In some embodiments, q is 2.

In some embodiments of Formulas (Id)-(Ig), r is 1 and p is 1. In some embodiments, r is 2 and p is 1. In some embodiments, r is 3 and p is 1.

In some embodiments, the present disclosure provides a compound of Formula (Ih) or a pharmaceutically acceptable salt thereof:

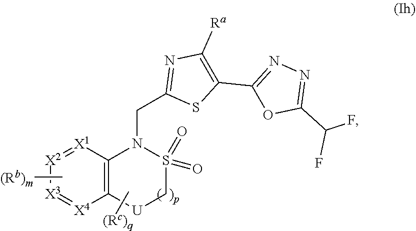

wherein:
U is NR^{d}, O, S, S(O), S(O)₂, CH₂, CHF, or CF₂;
X¹, X², X³, and X⁴ is each independently CH or N;
R^{a} is H, Me, or F;
R^{b} is each independently halo, alkyl, haloalkyl, alkoxy, haloalkoxy, —C(O)R^{e}, —C(O)OR^{e}, —C(O)N(R^{e})(R^{e′}), —SO₂R^{e}, cycloalkyl, heteroaryl, or heterocyclyl;
R^{c} is each independently F, alkyl, haloalkyl, alkoxy, or haloalkoxy, and/or two R^{c} groups taken together with the atoms to which they are attached form an optionally substituted C₃₋₇ cycloalkyl;
R^{d} is H, alkyl, acyl, sulfonyl, cycloalkyl, aryl, or heteroaryl;
R^{e} and R^{e′} is each independently H, alkyl, cycloalkyl, heterocyclyl, aryl, heteroaryl, —CH₂ cycloalkyl, —CH₂ heterocyclyl, —CH₂ aryl, or —CH₂ heteroaryl;
m is 0, 1, 2, or 3;
p is 0, 1, 2, or 3; and
q is 0, 1, or 2.

In some embodiments, the present disclosure provides a compound of Formula (Ii) or a pharmaceutically acceptable salt thereof:

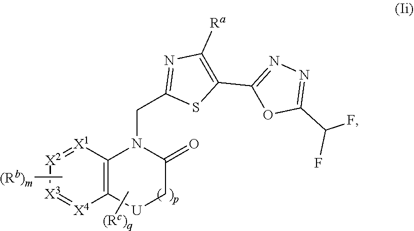

wherein:
U is NR^{d}, O, S, S(O), S(O)₂, CH₂, CHF, or CF₂;
X¹, X², X³, and X⁴ is each independently CH or N;
R^{a} is H, Me, or F;
R^{b} is each independently halo, alkyl, haloalkyl, alkoxy, haloalkoxy, —C(O)R^{e}, —C(O)OR^{e}, —C(O)N(R^{e})(R^{e′}), —SO₂R^{e}, cycloalkyl, heteroaryl, or heterocyclyl;
R^{c} is each independently F, alkyl, haloalkyl, alkoxy, or haloalkoxy, and/or two R^{c} groups taken together with the atoms to which they are attached form an optionally substituted C₃₋₇ cycloalkyl;
R^{d} is H, alkyl, —C(O)R^{e}, sulfonyl, cycloalkyl, aryl, or heteroaryl;
R^{e} and R^{e′} is each independently H, alkyl, cycloalkyl, heterocyclyl, aryl, heteroaryl, —CH₂ cycloalkyl, —CH₂ heterocyclyl, —CH₂ aryl, or —CH₂ heteroaryl;
m is 0, 1, 2, or 3;
p is 0, 1, 2, or 3; and
q is 0, 1, or 2.

In some embodiments, the present disclosure provides a compound of Formula (Ij) or a pharmaceutically acceptable salt thereof:

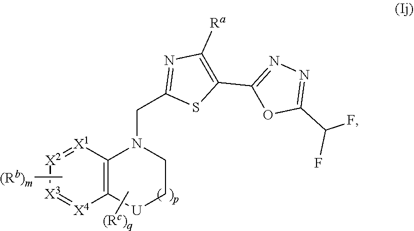

wherein:
U is NR^{d}, O, S, S(O), S(O)₂, CH₂, CHF, or CF₂;
X¹, X², X³, and X⁴ is each independently CH or N;
R^{a} is H, Me, or F;
R^{b} is each independently halo, alkyl, haloalkyl, alkoxy, haloalkoxy, —C(O)R^{e}, —C(O)OR^{e}, —C(O)N(R^{e})(R^{e′}), —SO₂R^{e}, cycloalkyl, heteroaryl, or heterocyclyl;
R^{c} is each independently F, alkyl, haloalkyl, alkoxy, or haloalkoxy, and/or two R^{c} groups taken together with the atoms to which they are attached form an optionally substituted C₃₋₇ cycloalkyl;
R^{d} is H, alkyl, —C(O)R^{e}, sulfonyl, cycloalkyl, aryl, or heteroaryl;
R^{e} and R^{e′} is each independently H, alkyl, cycloalkyl, heterocyclyl, aryl, heteroaryl, —CH₂ cycloalkyl, —CH₂ heterocyclyl, —CH₂ aryl, or —CH₂ heteroaryl;
m is 0, 1, 2, or 3;
p is 0, 1, 2, or 3; and
q is 0, 1, or 2.

In some embodiments of Formulas (Ih)-(Ij), NR^{d}, O, S, S(O)₂, or CH₂. In some embodiments, U is NR^{d}, O, S, or CH₂. In some embodiments, U is O or CH₂. In some embodiments, U is O. In some embodiments, U is CH₂. In some embodiments, U is S. In some embodiments, U is S(O)₂. In some embodiments, U is NR^{d}.

In some embodiments of Formulas (Ih)-(Ij), each of X¹, X², X³, and X⁴ is CH. In some embodiments, one of X¹, X², X³, and X⁴ is N. In some embodiments, two of X¹, X², X³, and X⁴ are N. In some embodiments, X¹ is N and each of X², X³, and X⁴ is CH. In some embodiments, X² is N and each of X¹, X³, and X⁴ is CH. In some embodiments, X³ is N and each of X¹, X², and X⁴ is CH. In some embodiments, X⁴ is N and each of X¹, X², and X³ is CH.

In some embodiments of Formulas (Ih)-(Ij), U is CH₂ and one of X¹, X², X³, and X⁴ is N. In some embodiments, U is CH₂, X¹ is N and each of X², X³, and X⁴ is CH. In some embodiments, U is CH₂, X² is N and each of X¹, X³, and X⁴ is CH. In some embodiments, U is CH₂, X³ is N and each of X¹, X², and X⁴ is CH. In some embodiments, U is CH₂, X⁴ is N and each of X¹, X², and X³ is CH. In some embodiments, p is 0. In some embodiments, p is 1.

In some embodiments of Formulas (Ih)-(Ij), U is O and one of X¹, X², X³, and X⁴ is N. In some embodiments, U is O, X¹ is N and each of X², X³, and X⁴ is CH. In some embodiments, U is O, X² is N and each of X¹, X³, and X⁴ is CH. In some embodiments, U is O, X³ is N and each of X¹, X², and X⁴ is CH. In some embodiments, U is O, X⁴ is N and each of X¹, X², and X³ is CH.

In some embodiments of Formulas (Ih)-(Ij), R^{a} is H. In some embodiments, R^{a} is F. In some embodiments, R^{a} is Me.

In some embodiments of Formulas (Ih)-(Ij), R^{b} is halo, alkyl, haloalkyl, alkyl, haloalkoxy, cycloalkyl, heterocyclyl, heteroaryl, or nitrile. In some embodiments, R^{b} is halo, alkyl, haloalkyl, alkyl, haloalkoxy, cycloalkyl, or nitrile. In some embodiments, the haloalkyl is selected from CF₃, CF₂CH₃, CHF₂, or CH₂F. In some embodiments, the alkyl is a-C₁₋₅ alkyl. In some embodiments, —C₁₋₅ alkyl is methyl, ethyl, propyl, i-propyl, butyl, or t-butyl. In some embodiments, the cycloalkyl is a C₃₋₆ cycloalkyl. In some embodiments, the heteroaryl is 5- or 6-membered heteroaryl having 1, 2, or 3 heteroatoms selected from N, O, and S. In some embodiments, the heterocyclyl is a 4- to 7-member heterocyclyl with 1 or 2 heteroatoms selected from N, O, and S. In some embodiments, the haloalkoxy is selected from OCF₃, OCHF₂, or OCH₂F. In some embodiments, the alkoxy is O-methyl, O-ethyl, O-propyl, O-i-propyl, O-butyl, or O-t-butyl.

In some embodiments of Formulas (Ih)-(Ij), R^{c} is F, C₁₋₅ alkyl, haloalkyl, C₁₋₅ alkoxy, haloalkoxy, acyl, sulfonyl, 5- or 6-membered heteroaryl, or C₃₋₆ heterocyclyl. In some embodiments, R^{c} is F, C₁₋₅ alkyl, haloalkyl, C₁₋₅ alkoxy, or haloalkoxy. In some embodiments, R is F or C₁₋₅ alkyl. In some embodiments, R^{c} is F or methyl. In some embodiments, R^{c} is F. In some embodiments, R^{c} is methyl. In some embodiments, the two R^{c} groups are attached to the same carbon atom, which can also be referred to as germinal substitution. In some embodiments, two R^{c} groups taken together with the atoms to which they are attached form an optionally substituted C₃₋₆ cycloalkyl. In some embodiments, two R^{c} groups taken together with the atoms to which they are attached form an optionally substituted cyclopropyl. In some embodiments, the optional substituent is one or more R^{b}, as defined above. In some embodiments, the optional substituent is selected from the group consisting of F, C₁₋₅ alkyl, C₁₋₅ alkoxy, CF₃, CF₂H, CFH₂, —OCF₃, —OCF₂H, —OCFH₂, —C(O)R^{e}, —C(O)OR^{e}, —C(O)N(R^{e})(R^{e′}), and —SO₂R^{e}. In some embodiments, the optional substituent is selected from the group consisting of F, C₁₋₅ alkyl, C₁₋₅ alkoxy, CF₃, CF₂H, CFH₂, —OCF₃, —OCF₂H, and —OCFH₂. In some embodiments, the optional substituent is F or C₁₋₅ alkyl. In some embodiments, the optional substituent is F. In some embodiments, the optional substituent is C₁₋₅ alkyl. In some embodiments, the C₁₋₅ alkyl is methyl. In some embodiments, the C₁₋₅ alkyl is ethyl. In some embodiments, the C₁₋₅ alkyl is propyl. In some embodiments, the C₁₋₅ alkyl is isopropyl. In some embodiments, two optional substituents are attached to the same carbon, which is also referred to as germinal substitution.

In some embodiments of Formulas (Ih)-(Ij), when U is NR^{d}, an R^{d} and R^{c} taken together with the atoms to which they are attached form a 5- to 7-membered heterocyclyl. In some embodiments, an R^{d} and R^{c} taken together with the atoms to which they are attached form a 6-membered heterocyclyl. In some embodiments, the heterocyclyl comprises 1 or 2 heteroatoms selected from N, O, and S.

In some embodiments, the present disclosure provides a compound of Formula (Ih-1), Formula (Ii-1), or Formula (Ij-1):

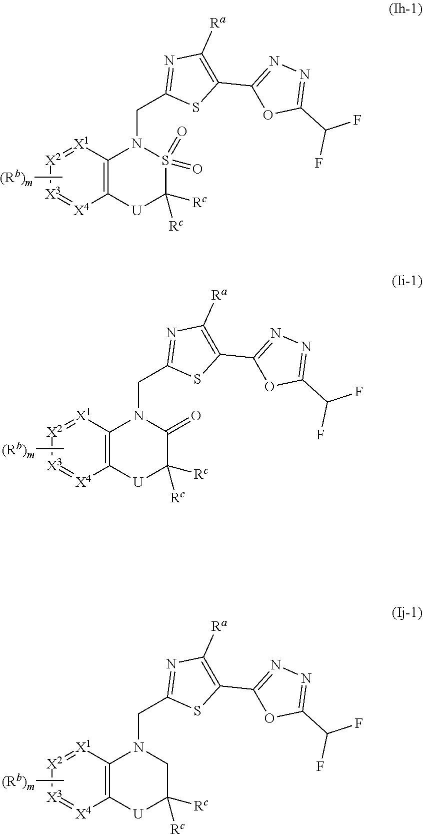

wherein R^{a}, R^{b}, R^{c}, X¹, X², X³, X⁴, U, and m are as defined above in Formula (Ih), Formula (Ii), and Formula (Ij).

In some embodiments of Formula (Ih-1), Formula (Ii-1), and Formula (Ij-1), each R^{c} is F. In some embodiments, each R^{c} is Me. In some embodiments, two R^{c} groups taken together with the carbon atoms to which they are attached form an optionally substituted C₃₋₆ cycloalkyl. In some embodiments, two R^{c} groups taken together with the carbon atoms to which they are attached form a cyclopropyl or cyclobutyl, each of which is optionally substituted. In some embodiments, two R^{c} groups taken together with the carbon atoms to which they are attached form an optionally substituted cyclopropyl. In some embodiments, the optional substituent is F or C₁₋₅ alkyl. In some embodiments, the optional substituent is F. In some embodiments, the optional substituent is C₁₋₅ alkyl. In some embodiments, the C₁₋₅ alkyl is methyl. In some embodiments, the C₁₋₅ alkyl is ethyl. In some embodiments, the C₁₋₅ alkyl is propyl. In some embodiments, the C₁₋₅ alkyl is isopropyl. In some embodiments, two optional substituents are attached to the same carbon, which is also referred to as germinal substitution.

In some embodiments, R^{d} is H, alkyl, or cycloalkyl. In some embodiments, R^{d} is H. In some embodiments, R^{d} is alkyl. In some embodiments, R^{d} is cycloalkyl. In some embodiments, alkyl is methyl, ethyl, propyl, isopropyl, or t-butyl. In some embodiments, the cycloalkyl is cyclopropyl, cyclopentyl, or cyclohexyl.

In some embodiments, m is 0, 1, or 2. In some embodiments, m is 0 or 1. In some embodiments, m is 0. In some embodiments, m is 1. In some embodiments, m is 2.

In some embodiments, p is 0, 1, or 2. In some embodiments, p is 0 or 1. In some embodiments, p is 1 or 2. In some embodiments, p is 0. In some embodiments, p is 1. In some embodiments, p is 2.

In some embodiments, q is 0 or 1. In some embodiments, q is 0. In some embodiments, q is 1. In some embodiments, q is 2.

In some embodiments, the HDAC6 inhibitor has the formula:

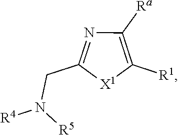

or a pharmaceutically acceptable salt thereof,
wherein:
X¹ is S;
R^{a} is selected from the group consisting of H, halogen, and C₁₋₃ alkyl;
R¹ is

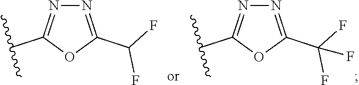 [SMILES: *c1nnc(C(F)F)o1 ; *c1nnc(C(F)(F)F)o1]

R² is selected from the group consisting of alkyl, alkoxy, and cycloalkyl, each of which is optionally substituted;
R³ is H or alkyl;
R⁴ is selected from the group consisting of alkyl, —(SO₂)R², —(SO₂)NR²R³, and —(CO)R²; and
R⁵ is aryl or heteroaryl; or R⁴ and R⁵ together with the atom to which they are attached form a heterocyclyl, each of which is optionally substituted;

In some embodiments, R^{a} is H.

In some embodiments, R¹ is

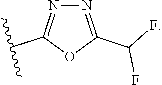 [SMILES: *c1nnc(C(F)F)o1]

In some embodiments, R⁴ is —(SO₂)R².

In some embodiments, —(SO₂)R² is —(SO₂)alkyl, —(SO₂)alkyleneheterocyclyl, —(SO₂)haloalkyl, —(SO₂)haloalkoxy, or —(SO₂)cycloalkyl.

In some embodiments, R⁵ is heteroaryl.

In some embodiments, the heteroaryl is a 5- to 6-membered heteroaryl

In some embodiments, the 5- to 6-membered heteroaryl is selected from the group consisting of

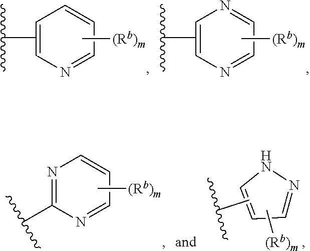

wherein R^{b} is halogen, alkyl, alkoxy, cycloalkyl, —CN, haloalkyl, or haloalkoxy; and m is 0 or 1.

In some embodiments, R^{b} is F, Cl, —CH₃, —CH₂CH₃, —CF₃, —CHF₂, —CF₂CH₃, —CN, —OCH₃, —OCH₂CH₃, —OCH(CH₃)₂, —OCF₃, —OCHF₂, —OCH₂CF₂H, and cyclopropyl.

In some embodiments, the aryl is selected from the group consisting of phenyl, 3-chlorophenyl, 3-chloro-4-fluorophenyl, 3-trifluoromethylphenyl, 3,4-difluorophenyl, and 2,6-difluorophenyl.

In some embodiments, the HDAC6 inhibitor has the Formula (Ik):

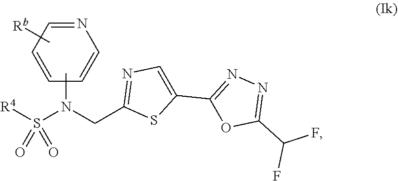

or a pharmaceutically acceptable salt thereof,
wherein:
R^{b} is H, halogen, alkyl, cycloalkyl, —CN, haloalkyl, or haloalkoxy; and
R⁴ is alkyl, alkoxy, haloalkyl, or cycloalkyl, each of which is optionally substituted.

In some embodiments, R^{b} is H, halogen, haloalkyl, or haloalkoxy.

In some embodiments, R⁴ is optionally substituted alkyl or cycloalkyl.

In some embodiments, the HDAC6 inhibitor has the structure:

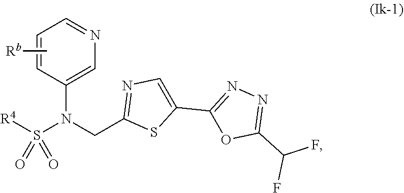

or a pharmaceutically acceptable salt thereof,
   wherein:
R^{b} is H, halogen, alkyl, cycloalkyl, —CN, haloalkyl, or haloalkoxy; and
R⁴ is alkyl, alkoxy, haloalkyl, or cycloalkyl, each of which is optionally substituted.

In some embodiments, R^{b} is H, halogen, haloalkyl, or haloalkoxy.

In some embodiments, R⁴ is optionally substituted alkyl or cycloalkyl.

In some embodiments, R⁴ is alkyl.

In some embodiments, the HDAC6 inhibitor has the structure:

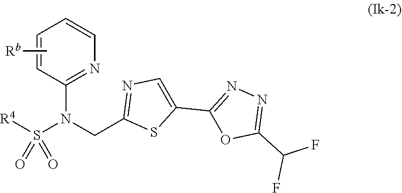

or a pharmaceutically acceptable salt thereof,
   wherein:
R^{b} is H, halogen, alkyl, cycloalkyl, —CN, haloalkyl, or haloalkoxy; and
R⁴ is alkyl, alkoxy, haloalkyl, or cycloalkyl, each of which is optionally substituted.

In some embodiments, R^{b} is H, halogen, haloalkyl, or haloalkoxy.

In some embodiments, R⁴ is optionally substituted alkyl.

In some embodiments, the HDAC6 inhibitor is a compound having the formula:

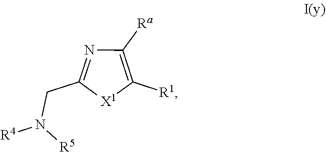

or a pharmaceutically acceptable salt thereof,
wherein:
X¹ is S;
R^{a} is selected from the group consisting of H, halogen, and C₁₋₃ alkyl;
R¹ is

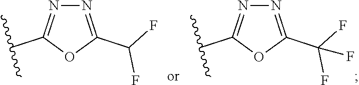 [SMILES: *c1nnc(C(F)F)o1 ; *c1nnc(C(F)(F)F)o1]

R² is selected from the group consisting of alkyl, alkoxy, and cycloalkyl, each of which is optionally substituted;
R³ is H or alkyl;
R⁴ is selected from the group consisting of alkyl, —(SO₂)R², —(SO₂)NR²R³, and —(CO)R²; and
R⁵ is aryl or heteroaryl; or R⁴ and R⁵ together with the atom to which they are attached form a heterocyclyl, each of which is optionally substituted.

In some embodiments of Formula I(y), R^{a} is H.

In some embodiments of Formula I(y), R¹ is

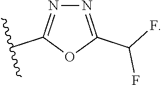 [SMILES: *c1nnc(C(F)F)o1]

In some embodiments of Formula I(y), R⁴ is —(SO₂)R².

In some embodiments of Formula I(y), —(SO₂)R² is —(SO₂)alkyl, —(SO₂)alkyleneheterocyclyl, —(SO₂)haloalkyl, —(SO₂)haloalkoxy, or —(SO₂)cycloalkyl.

In some embodiments of Formula I(y), R⁵ is heteroaryl.

In some embodiments of Formula I(y), the heteroaryl is a 5- to 6-membered heteroaryl.

In some embodiments of Formula I(y), the 5- to 6-membered heteroaryl is selected from the group consisting of

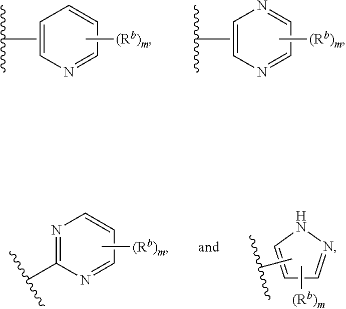

wherein R is halogen, alkyl, alkoxy, cycloalkyl, —CN, haloalkyl, or haloalkoxy; and m is 0 or 1.

In some embodiments of Formula I(y), R^{b} is F, Cl, —CH₃, —CH₂CH₃, —CF₃, —CHF₂, —CF₂CH₃, —CN, —OCH₃, —OCH₂CH₃, —OCH(CH₃)₂, —OCF₃, —OCHF₂, —OCH₂CF₂H, and cyclopropyl.

In some embodiments of Formula I(y), the aryl is selected from the group consisting of phenyl, 3-chlorophenyl, 3-chloro-4-fluorophenyl, 3-trifluoromethylphenyl, 3,4-difluorophenyl, and 2,6-difluorophenyl.

In some embodiments, the HDAC6 inhibitor has the structure:

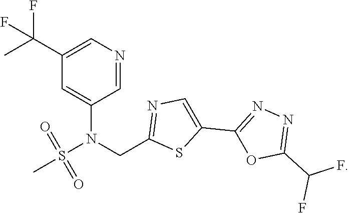 [SMILES: CC(F)(F)c1cncc(N(Cc2ncc(-c3nnc(C(F)F)o3)s2)S(C)(=O)=O)c1]

In some embodiments, the HDAC6 inhibitor has the structure:

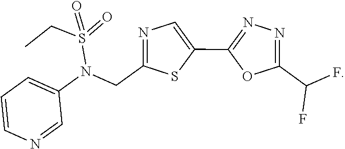 [SMILES: CCS(=O)(=O)N(Cc1ncc(-c2nnc(C(F)F)o2)s1)c1cccnc1]

In some embodiments, the HDAC6 inhibitor has the structure:

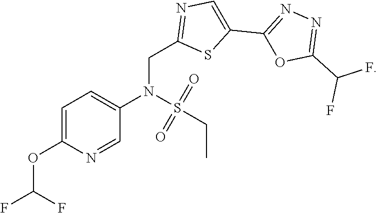 [SMILES: CCS(=O)(=O)N(Cc1ncc(-c2nnc(C(F)F)o2)s1)c1ccc(OC(F)F)nc1]

In some embodiments, the HDAC6 inhibitor has the structure:

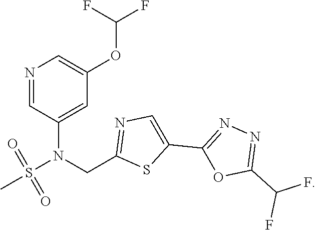 [SMILES: CS(=O)(=O)N(Cc1ncc(-c2nnc(C(F)F)o2)s1)c1cncc(OC(F)F)c1]

In some embodiments, the HDAC6 inhibitor has the structure:

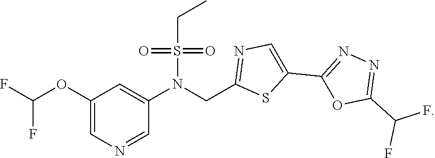 [SMILES: CCS(=O)(=O)N(Cc1ncc(-c2nnc(C(F)F)o2)s1)c1cncc(OC(F)F)c1]

In some embodiments, the HDAC6 inhibitor has the structure:

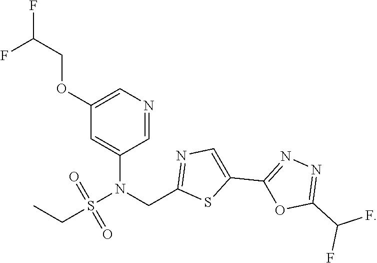 [SMILES: CCS(=O)(=O)N(Cc1ncc(-c2nnc(C(F)F)o2)s1)c1cncc(OCC(F)F)c1]

In some embodiments, the HDAC6 inhibitor has the structure:

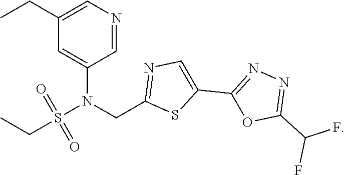 [SMILES: CCc1cncc(N(Cc2ncc(-c3nnc(C(F)F)o3)s2)S(=O)(=O)CC)c1]

In some embodiments, the HDAC6 inhibitor has the structure:

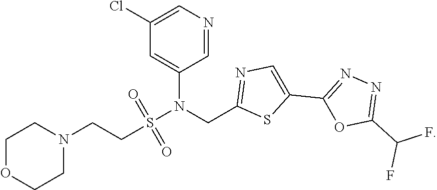 [SMILES: O=S(=O)(CCN1CCOCC1)N(Cc1ncc(-c2nnc(C(F)F)o2)s1)c1cncc(Cl)c1]

In some embodiments, the HDAC6 inhibitor has the structure:

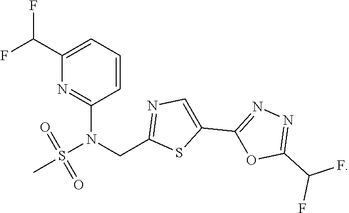 [SMILES: CS(=O)(=O)N(Cc1ncc(-c2nnc(C(F)F)o2)s1)c1cccc(C(F)F)n1]

In some embodiments, the HDAC6 inhibitor has the structure:

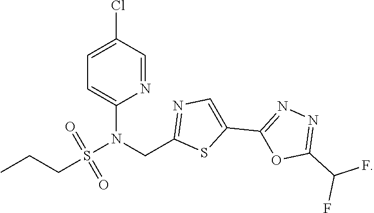 [SMILES: CCCS(=O)(=O)N(Cc1ncc(-c2nnc(C(F)F)o2)s1)c1ccc(Cl)cn1]

In some embodiments, the HDAC6 inhibitor is TYA-018 or an analog thereof. The structure of TYA-018 is:

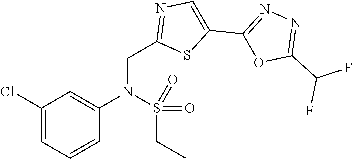 [SMILES: CCS(=O)(=O)N(Cc1ncc(-c2nnc(C(F)F)o2)s1)c1cccc(Cl)c1]

Analogs of TYA-018 include, without limitation, the compounds listed in Table 2.

TABLE 2

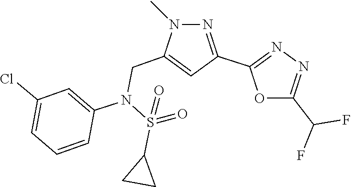 [SMILES: Cn1nc(-c2nnc(C(F)F)o2)cc1CN(c1cccc(Cl)c1)S(=O)(=O)C1CC1]

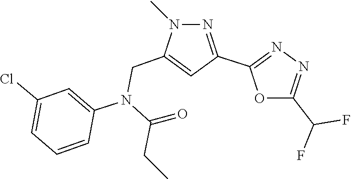 [SMILES: CCC(=O)N(Cc1cc(-c2nnc(C(F)F)o2)nn1C)c1cccc(Cl)c1]

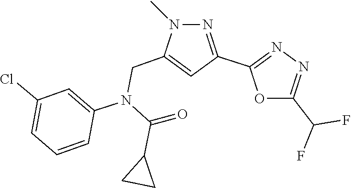 [SMILES: Cn1nc(-c2nnc(C(F)F)o2)cc1CN(C(=O)C1CC1)c1cccc(Cl)c1]

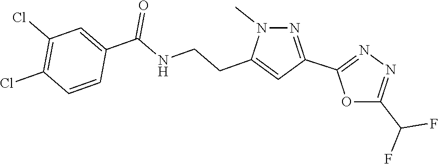 [SMILES: Cn1nc(-c2nnc(C(F)F)o2)cc1CCNC(=O)c1ccc(Cl)c(Cl)c1]

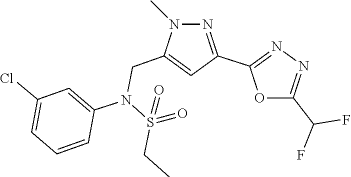 [SMILES: CCS(=O)(=O)N(Cc1cc(-c2nnc(C(F)F)o2)nn1C)c1cccc(Cl)c1]

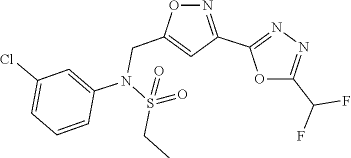 [SMILES: CCS(=O)(=O)N(Cc1cc(-c2nnc(C(F)F)o2)no1)c1cccc(Cl)c1]

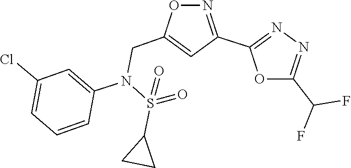 [SMILES: O=S(=O)(C1CC1)N(Cc1cc(-c2nnc(C(F)F)o2)no1)c1cccc(Cl)c1]

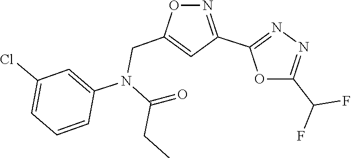 [SMILES: CCC(=O)N(Cc1cc(-c2nnc(C(F)F)o2)no1)c1cccc(Cl)c1]

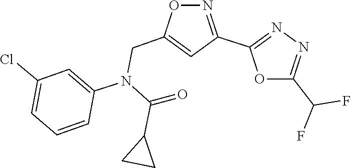 [SMILES: O=C(C1CC1)N(Cc1cc(-c2nnc(C(F)F)o2)no1)c1cccc(Cl)c1]

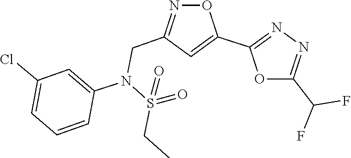 [SMILES: CCS(=O)(=O)N(Cc1cc(-c2nnc(C(F)F)o2)on1)c1cccc(Cl)c1]

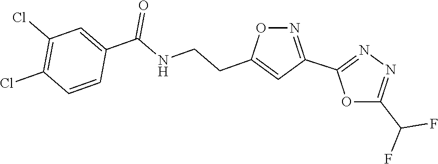 [SMILES: O=C(NCCc1cc(-c2nnc(C(F)F)o2)no1)c1ccc(Cl)c(Cl)c1]

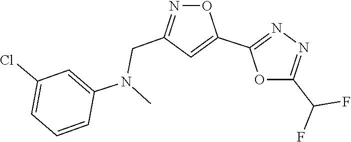 [SMILES: CN(Cc1cc(-c2nnc(C(F)F)o2)on1)c1cccc(Cl)c1]

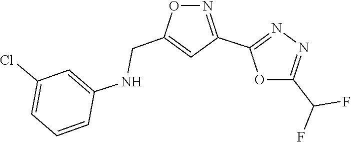 [SMILES: FC(F)c1nnc(-c2cc(CNc3cccc(Cl)c3)on2)o1]

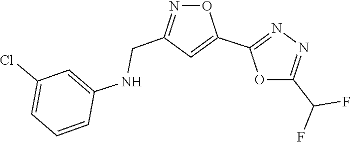 [SMILES: FC(F)c1nnc(-c2cc(CNc3cccc(Cl)c3)no2)o1]

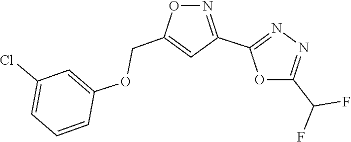 [SMILES: FC(F)c1nnc(-c2cc(COc3cccc(Cl)c3)on2)o1]

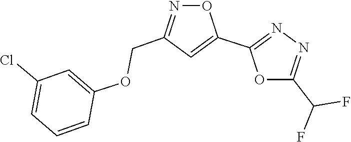 [SMILES: FC(F)c1nnc(-c2cc(COc3cccc(Cl)c3)no2)o1]

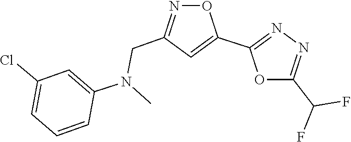 [SMILES: CN(Cc1cc(-c2nnc(C(F)F)o2)on1)c1cccc(Cl)c1]

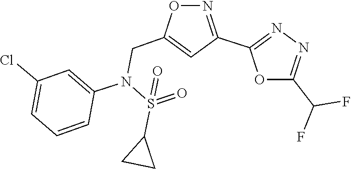 [SMILES: O=S(=O)(C1CC1)N(Cc1cc(-c2nnc(C(F)F)o2)no1)c1cccc(Cl)c1]

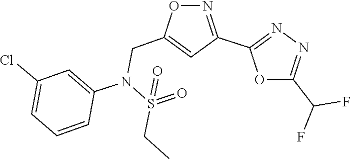 [SMILES: CCS(=O)(=O)N(Cc1cc(-c2nnc(C(F)F)o2)no1)c1cccc(Cl)c1]

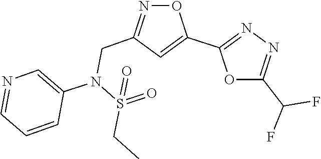 [SMILES: CCS(=O)(=O)N(Cc1cc(-c2nnc(C(F)F)o2)on1)c1cccnc1]

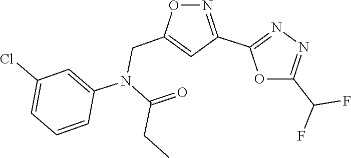 [SMILES: CCC(=O)N(Cc1cc(-c2nnc(C(F)F)o2)no1)c1cccc(Cl)c1]

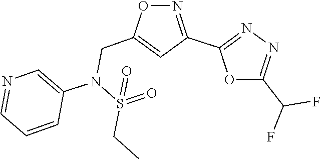 [SMILES: CCS(=O)(=O)N(Cc1cc(-c2nnc(C(F)F)o2)no1)c1cccnc1]

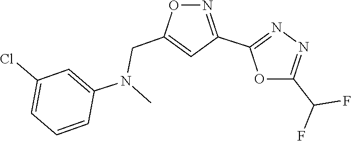 [SMILES: CN(Cc1cc(-c2nnc(C(F)F)o2)no1)c1cccc(Cl)c1]

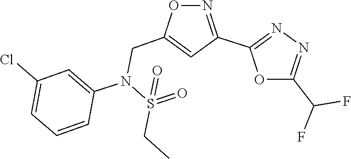 [SMILES: CCS(=O)(=O)N(Cc1cc(-c2nnc(C(F)F)o2)no1)c1cccc(Cl)c1]

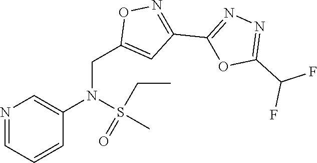

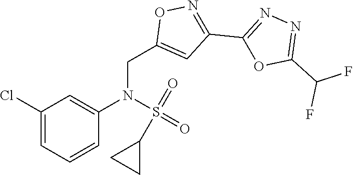 [SMILES: O=S(=O)(C1CC1)N(Cc1cc(-c2nnc(C(F)F)o2)no1)c1cccc(Cl)c1]

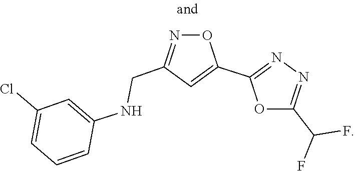 [SMILES: FC(F)c1nnc(-c2cc(CNc3cccc(Cl)c3)no2)o1]

### 5-Fluoronicotinamide Derivatives

In some embodiments, the HDAC6 inhibitor is a 5-fluoronicotinamide derivative. Illustrative derivatives that may be used as HDAC6 inhibitors include those described herein and those described in Int'l Pat. Appl. Pub. No. PCT/US2020/054134, published as WO2021067859A1, the content of which is incorporated by reference herein in its entirety. PCT/US2020/054134, published as WO2021067859A1, also describes methods of synthesis of such compounds, which are specifically incorporated by reference herein.

In some embodiments, the HDAC6 inhibitor is a compound of Formula (II):

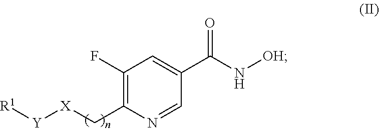

wherein
n is 0 or 1;
X is O, NR⁴, or CR⁴R^{4′};
Y is a bond, CR²R³ or S(O)₂;
R¹ is selected from the group consisting of H, amido, carbocyclyl, heterocyclyl, aryl, and heteroaryl;
R² and R³ are independently selected from the group consisting of H, halogen, alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or
R¹ and R² taken together with the carbon atom to which they are attached form a carbocyclyl or heterocyclyl; or
R² and R³ taken together with the carbon atom to which they are attached form a carbocyclyl or heterocyclyl; and
R⁴ and R^{4′} are each independently selected from the group consisting of H, alkyl, —CO₂-alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or
R⁴ and R^{4′} taken together with the carbon atom to which they are attached form a carbocyclyl or heterocyclyl;
wherein each alkyl, carbocyclyl, heterocyclyl, aryl, and heteroaryl is independently optionally substituted with one or more substituents selected from the group consisting of halogen, haloalkyl, oxo, hydroxy, alkoxy, —OCH₃, —CO₂CH₃, —C(O)NH(OH), —CH₃, morpholine, and —C(O)N-cyclopropyl.

### Pharmaceutical Compositions and Kits

In various embodiments of the present disclosure, pharmaceutical compositions comprising one or more HDAC6 inhibitors disclosed herein, or a pharmaceutically acceptable salt thereof, or a pharmaceutically acceptable solvate, hydrate, tautomer, N-oxide, or salt thereof, and a pharmaceutically acceptable excipient or adjuvant is provided. The pharmaceutically acceptable excipients and adjuvants are added to the composition or formulation for a variety of purposes. In some embodiments, a pharmaceutical composition comprising one or more compounds disclosed herein, or a pharmaceutically acceptable solvate, hydrate, tautomer, N-oxide, or salt thereof, further comprises a pharmaceutically acceptable carrier. In some embodiments, a pharmaceutically acceptable carrier includes a pharmaceutically acceptable excipient, binder, and/or diluent. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, hydroxymethylcellulose and polyvinylpyrrolidone.

In some embodiments, the HDAC6 inhibitor in the pharmaceutical composition described herein is one or more compounds of Formula (I), Formula (Ia), Formula (Ib), Formula (Ic), Formula (Id), Formula (Id-1), Formula (Id-2), Formula (Id-3), Formula (Id-4), Formula (Ie), Formula (le-1), Formula (If), Formula (If-1), Formula (Ig), Formula (Ig-1), Formula (Ih), Formula (Ih-1), Formula (Ii), Formula (Ii-1), Formula (Ij), Formula (Ij-1), Formula (Ik), Formula (Ik-1), Formula (Ik-2), Formula (Ik-3), Formula I(y), or Formula (II). In some embodiments, the HDAC6 inhibitor in the pharmaceutical composition described herein is a compound of Formula (I). In some embodiments, the HDAC6 inhibitor in the pharmaceutical composition described herein is a compound of Formula (Ic). In some embodiments, the HDAC6 inhibitor in the pharmaceutical composition described herein is a compound of Formula (Ik). In some embodiments, the HDAC6 inhibitor in the pharmaceutical composition described herein is a compound of Formula I(y) (which may also be referenced herein as Formula (Iy)).

In another aspect, the disclosure provides an HDAC6 inhibitor for use in a method for treating dilated cardiomyopathy.

In another aspect, the disclosure provides a kit, comprising an HDAC6 inhibitor, or pharmaceutical composition thereof, and instructions for use in a method for treating dilated cardiomyopathy.

In another aspect, the disclosure provides use of an HDAC6 inhibitor in treating dilated cardiomyopathy.

### Screening Methods

In another aspect, the disclosure provides method of identifying a compound for treatment of dilated cardiomyopathy, comprising contacting a cell culture comprising cells having an inactivating mutation in BAG3 with each member of a plurality of candidate compounds; and selecting a compound that reduces sarcomere damage in the cells. In another aspect, the disclosure provides method of identifying a compound for treatment of dilated cardiomyopathy, comprising contacting a cell culture comprising cells having an inactivating mutation in MLP (CSRP3) with each member of a plurality of candidate compounds; and selecting a compound that reduces sarcomere damage in the cells.

In another aspect, the disclosure provides method of treating dilated cardiomyopathy in a subject in need thereof, comprising identifying a compound by contacting a cell culture comprising cells having an inactivating mutation in BAG3 with each member of a plurality of candidate compounds; and selecting a selected compound as reducing sarcomere damage; and administering a therapeutically effective amount of the selected compound to the subject. In another aspect, the disclosure provides method of treating dilated cardiomyopathy in a subject in need thereof, comprising identifying a compound by contacting a cell culture comprising cells having an inactivating mutation in MLP (CSRP3) with each member of a plurality of candidate compounds; and selecting a selected compound as reducing sarcomere damage; and administering a therapeutically effective amount of the selected compound to the subject.

### Methods of Administration and Patient Populations to be Treated

The HDAC6 inhibitors described herein (and pharmaceutical compositions comprising such HDAC6 inhibitors) can be administered to a subject by any suitable means disclosed herein or known in the art.

In some embodiments, the administration of an HDAC6 inhibitor is oral administration. In some embodiments, the method comprises orally administering to a subject an HDAC6 inhibitor of Formula (I), Formula (Ia), Formula (Ib), Formula (Ic), Formula (Id), Formula (Id-1), Formula (Id-2), Formula (Id-3), Formula (Id-4), Formula (Ie), Formula (Ie-1), Formula (If), Formula (If-1), Formula (Ig), Formula (Ig-1), Formula (Ih), Formula (Ih-1), Formula (Ii), Formula (Ii-1), Formula (Ij), Formula (Ij-1), Formula (Ik), Formula (Ik-1), Formula (Ik-2), Formula (Ik-3), Formula I(y), or Formula (II). In some embodiments, the method comprises orally administering to a subject an HDAC6 inhibitor of Formula (I). In some embodiments, the method comprises orally administering to a subject an HDAC6 inhibitor of Formula (Ic). In some embodiments, the method comprises orally administering to a subject an HDAC6 inhibitor of Formula (Ik). In some embodiments, the method comprises orally administering to a subject an HDAC6 inhibitor of Formula I(y). In some embodiments, the method comprises orally administering to a subject an HDAC6 inhibitor of Formula (II). In some embodiments, oral administration is by means of a tablet or capsule. In some embodiments, a human is orally administered an HDAC6 inhibitor described herein (or a pharmaceutical composition thereof).

Various dosing schedules of the HDAC6 inhibitors described herein (and pharmaceutical compositions comprising such HDAC6 inhibitors) are contemplated including single administration or multiple administrations over a period of time.

In some embodiments, an HDAC6 inhibitor described herein (or a pharmaceutical composition comprising the inhibitor) is administered twice a day, once a day, once in two days, once in three days, once a week, once in two weeks, once in three weeks or once a month. In some embodiments, an HDAC6 inhibitor described herein (or a pharmaceutical composition comprising the inhibitor) is administered once a day.

In some embodiments, an HDAC6 inhibitor described herein (or a pharmaceutical composition comprising the inhibitor) is administered a single time. In some embodiments, an HDAC6 inhibitor described herein (or a pharmaceutical composition comprising the inhibitor) is administered over a period of time, for example, for (or longer than) 1 week, 2 weeks, 3 weeks, 4 weeks, 5 weeks, 6 weeks, 7 weeks, 8 weeks, 9 weeks, 10 weeks, 11 weeks, 3 months, 4 months, 5 months, 6 months, 7 months, 8 months, 9 months, 10 months, 11 months, 1 year. In some embodiments, the subject being treated is administered an HDAC6 inhibitor described herein (or a pharmaceutical composition comprising the inhibitor) for at least 1 month, at least 6 weeks, at least 2 months, at least 3 months, or at least 6 months. In some embodiments, the subject being treated is administered an HDAC6 inhibitor described herein (or a pharmaceutical composition comprising the inhibitor) for less than 1 month, 6 weeks, 2 months, 3 months, or 6 months.

The appropriate dosage of an HDAC6 inhibitor described herein for use in the methods described herein will depend on the type of inhibitor used, the condition of the subject (e.g., age, body weight, health), the responsiveness of the subject, other medications used by the subject, and other factors to be considered at the discretion of the medical practitioner performing the treatment.

In some embodiments, an HDAC6 inhibitor described herein is administered to the subject in the amount in the range from 1 mg to 500 mg per day. In some embodiments, an HDAC6 inhibitor described herein is administered to a human orally in the amount in the range from 1 mg to 500 mg per day. In some embodiments, an HDAC6 inhibitor described herein is administered to a human orally in a single dose in the amount in the range from 1 mg to 500 mg. In some embodiments, an HDAC6 inhibitor described herein is administered to a human orally in the amount in the range from 1 mg to 500 mg once a day, e.g., over a course of treatment (e.g., for 1 week, 2 weeks, 3 weeks, 4 weeks, 5 weeks, 6 weeks, 7 weeks, 8 weeks, 9 weeks, 10 weeks, 11 weeks, 3 months, 4 months, 5 months, 6 months, 7 months, 8 months, 9 months, 10 months, 11 months, 1 year, or longer).

In some embodiments, the HDAC6 inhibitors described herein (and pharmaceutical compositions comprising such HDAC6 inhibitors) can be administered to a subject in combination with another medication or therapy. In some embodiments, two or three different HDAC6 inhibitors (e.g., out of those described herein) can be administered to a subject. In some embodiments, one or more of the HDAC6 inhibitors described herein (and pharmaceutical compositions comprising such HDAC6 inhibitors) can be administered to a subject in combination with one or more therapy different from said one or more HDAC6 inhibitor(s), where the therapy is a cardioprotective therapy, a therapy for a heart condition (e.g., heart failure) and/or a therapy for DCM. The additional therapy can be any cardioprotective therapy, heart condition therapy (e.g., heart failure) therapy or anti-DCM therapy known in the art. In some embodiments, an HDAC6 inhibitor described herein (or a pharmaceutical composition comprising such HDAC6 inhibitor) is administered to a subject in combination with another anti-DCM therapy. In some embodiments, an HDAC6 inhibitor described herein (or a pharmaceutical composition comprising such HDAC6 inhibitor) is administered to a subject in combination with a cardioprotective therapy. In some embodiments, an HDAC6 inhibitor described herein (or a pharmaceutical composition comprising such HDAC6 inhibitor) is administered to a subject in combination with an ACE inhibitor. In some embodiments, an HDAC6 inhibitor described herein (or a pharmaceutical composition comprising such HDAC6 inhibitor) is administered to a subject in combination with a beta blocker. In some embodiments, an HDAC6 inhibitor described herein (or a pharmaceutical composition comprising such HDAC6 inhibitor) is administered to a subject before, at the same time, or after the additional therapy (such as a cardioprotective or anti-DCM therapy, e.g., an ACE inhibitor or a beta blocker). In some embodiments, the subject being treated in accordance with the methods described herein has not received an anti-DCM therapy, a cardioprotective therapy, and/or a heart condition (e.g., heart failure) therapy.

In some embodiments provided herein are kits comprising an HDAC6 inhibitor described herein (or a pharmaceutical composition comprising the same) and one or more additional agents (e.g., an additional agent for the treatment of DCM or a cardioprotective agent). In some embodiments, provided herein are kits comprising (i) an HDAC6 inhibitor (e.g., in a therapeutically effective amount), and (ii) one or more additional agents, such as an ACE inhibitor, a beta blocker, or another agent for the treatment of DCM or cardioprotection (e.g., in a therapeutically effective amount).

In some embodiments, the subject is a human. In some embodiments, the human is an adult human. In some embodiments, the subject is a male. In some embodiments, the subject is a female.

In some embodiments, the subject has (e.g., has symptoms of or was diagnosed with) cardiomyopathy. In some embodiments, the cardiomyopathy is genetic cardiomyopathy. In some embodiments, the cardiomyopathy is non-genetic cardiomyopathy. In some embodiments, the subject has (e.g., has symptoms of or was diagnosed with) DCM. In some embodiments, the subject has (e.g., has symptoms of or was diagnosed with) familial DCM. In some embodiments, the subject has (e.g., has symptoms of or was diagnosed with) non-familial DCM. In some embodiments, the subject has (e.g., has symptoms of or was diagnosed with) idiopathic DCM. In some embodiments, the subject has (e.g., has symptoms of or was diagnosed with) DCM with reduced ejection fraction.

In some embodiments, the subject has a deleterious mutation in BAG3 (e.g., a deletion of BAG3 or a mutation resulting in inactivation of BAG3). In some embodiments, the subject has a deleterious mutation in MLP also known as CSRP3 (e.g., a deletion of MLP or a mutation resulting in inactivation of MLP).

### Numbered Embodiments of the Invention

1. A method of treating or preventing dilated cardiomyopathy in a subject in need thereof, comprising administering to the subject a HDAC6 inhibitor.

2. The method of embodiment 1, wherein the HDAC6 inhibitor is fluoroalkyl-oxadiazole derivative.

3. The method of embodiment 2, wherein the HDAC6 inhibitor is fluoroalkyl-oxadiazole derivative according to the following Formula:

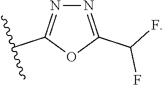 [SMILES: *c1nnc(C(F)F)o1]

4. The method of embodiment 1, wherein the HDAC6 inhibitor is a compound according to Formula (I):

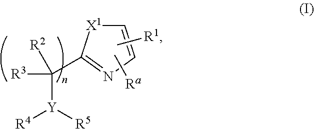

or pharmaceutically acceptable salt thereof, wherein
R¹ is selected from the group consisting of

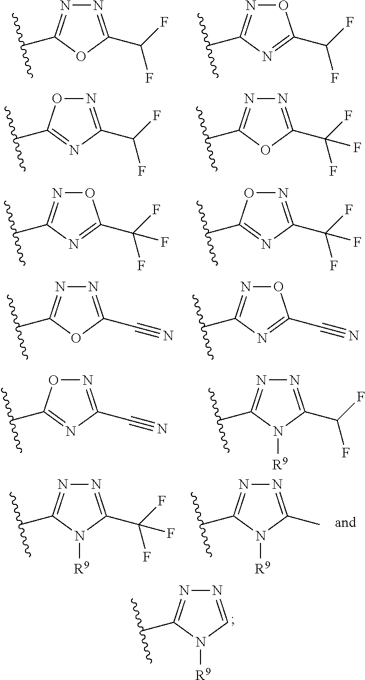

R^{a} is selected from the group consisting of H, halo, C₁₋₃ alkyl, cycloalkyl, haloalkyl, and alkoxy;
R² and R³ are independently selected from the group consisting of H, halogen, alkoxy, haloalkyl, aryl, heteroaryl, alkyl, and cycloalkyl each of which is optionally substituted, or R² and R³ together with the atom to which they are attached form a cycloalkyl or heterocyclyl;
R⁴ and R⁵ are independently selected from the group consisting of H, —(SO₂)R², —(SO₂)NR²R³, —(CO)R², —(CONR²R³), aryl, arylheteroaryl, alkylenearyl, heteroaryl, cycloalkyl, heterocyclyl, alkyl, haloalkyl, and alkoxy, each of which is optionally substituted, or R⁴ and R⁵ together with the atom to which they are attached form a cycloalkyl or heterocyclyl, each of which is optionally substituted;
R⁹ is selected from the group consisting of H, C₁-C₆ alkyl, haloalkyl, cycloalkyl and heterocyclyl;
X¹ is selected from the group consisting of S, O, NH and NR⁶, wherein R⁶ is selected from the group consisting of C₁-C₆ alkyl, alkoxy, haloalkyl, cycloalkyl and heterocyclyl;
Y is selected from the group consisting of CR², O, N, S, SO, and SO₂, wherein when Y is O, S, SO, or SO₂, R⁵ is not present and when R⁴ and R⁵ together with the atom to which they are attached form a cycloalkyl or heterocyclyl, Y is CR² or N; and
n is selected from 0, 1, and 2.

5. The method of embodiment 4, wherein the HDAC6 inhibitor is selected from the group consisting of:

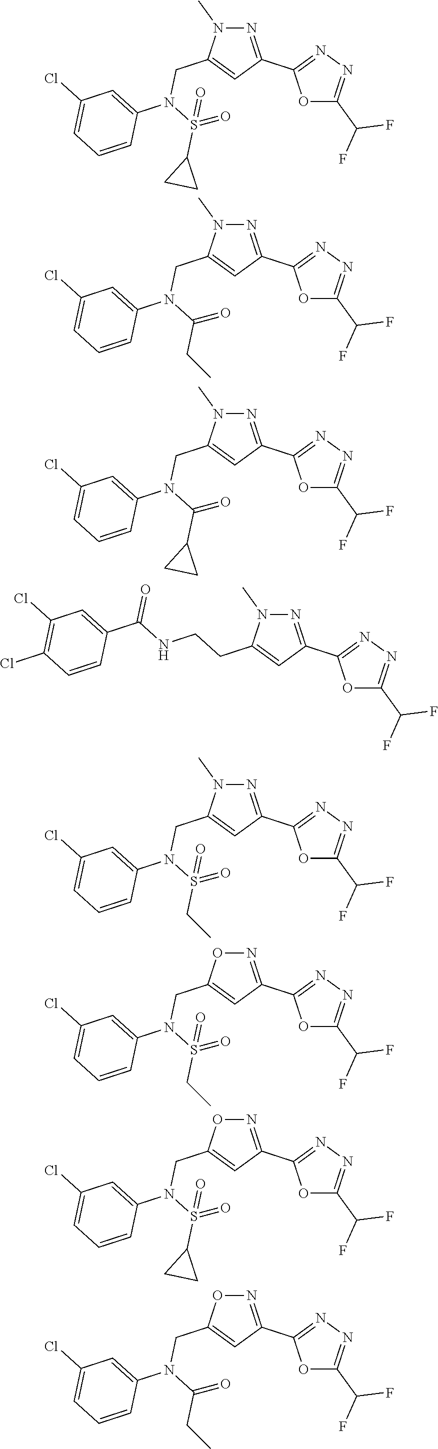

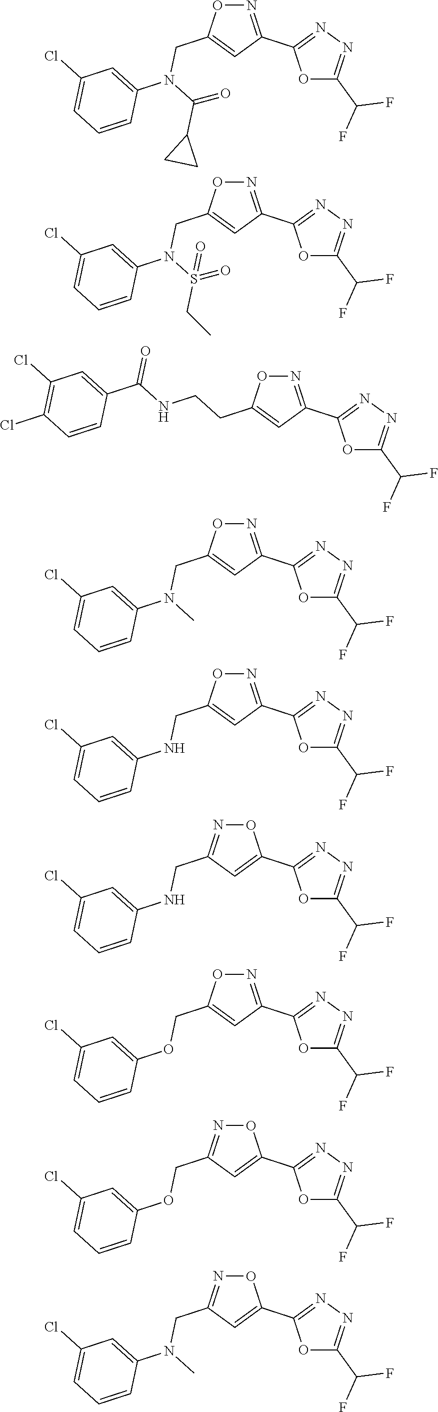

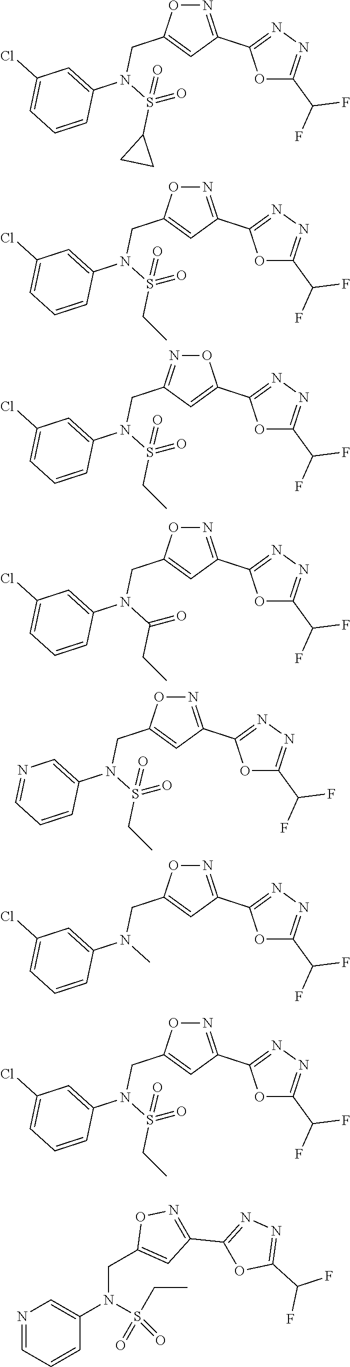

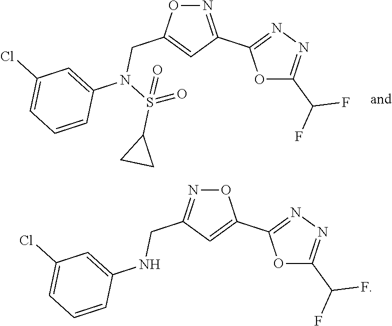

6. The method of embodiment 4, wherein the HDAC6 inhibitor is selected from the group consisting of:

| Cmpd | Structure/Name |
|---|---|
| I-1 | 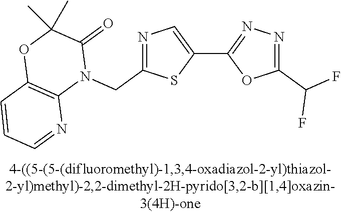 [SMILES: CC1(C)Oc2cccnc2N(Cc2ncc(-c3nnc(C(F)F)o3)s2)C1=O] |
| I-2 | 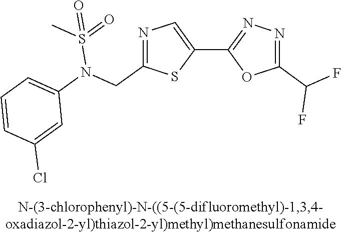 [SMILES: CS(=O)(=O)N(Cc1ncc(-c2nnc(C(F)F)o2)s1)c1cccc(Cl)c1] |
| I-3 | 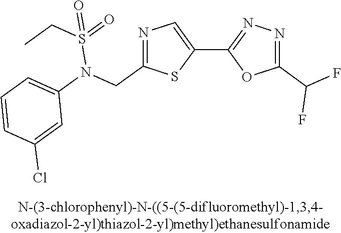 [SMILES: CCS(=O)(=O)N(Cc1ncc(-c2nnc(C(F)F)o2)s1)c1cccc(Cl)c1] |
| I-4 | 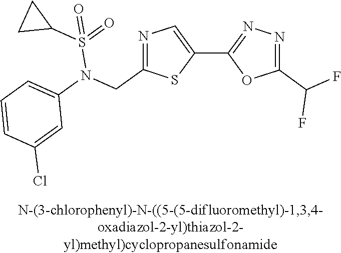 [SMILES: O=S(=O)(C1CC1)N(Cc1ncc(-c2nnc(C(F)F)o2)s1)c1cccc(Cl)c1] |
| I-5 | 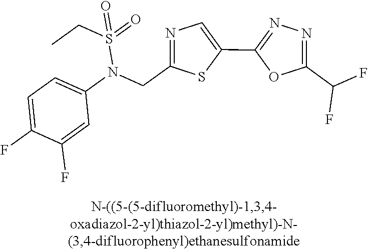 [SMILES: CCS(=O)(=O)N(Cc1ncc(-c2nnc(C(F)F)o2)s1)c1ccc(F)c(F)c1] |
| I-6 | 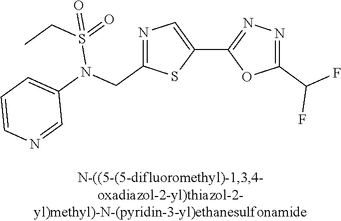 [SMILES: CCS(=O)(=O)N(Cc1ncc(-c2nnc(C(F)F)o2)s1)c1cccnc1] |
| I-7 | 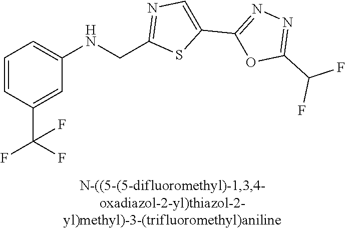 [SMILES: FC(F)c1nnc(-c2cnc(CNc3cccc(C(F)(F)F)c3)s2)o1] |
| I-8 | 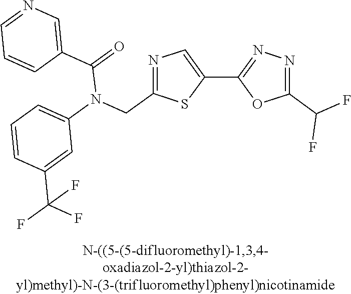 [SMILES: O=C(c1cccnc1)N(Cc1ncc(-c2nnc(C(F)F)o2)s1)c1cccc(C(F)(F)F)c1] |
| I-9 | 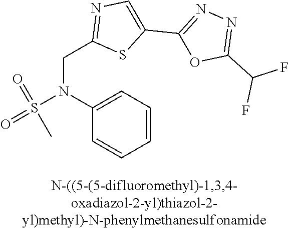 [SMILES: CS(=O)(=O)N(Cc1ncc(-c2nnc(C(F)F)o2)s1)c1ccccc1] |
| I-10 | 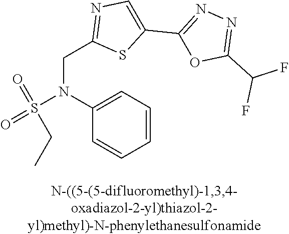 [SMILES: CCS(=O)(=O)N(Cc1ncc(-c2nnc(C(F)F)o2)s1)c1ccccc1] |
| I-12 | 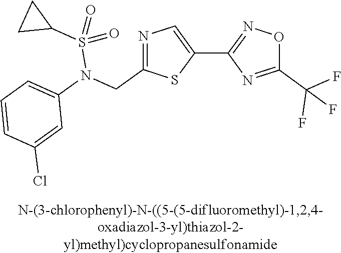 [SMILES: O=S(=O)(C1CC1)N(Cc1ncc(-c2noc(C(F)(F)F)n2)s1)c1cccc(Cl)c1] |
| I-13 | 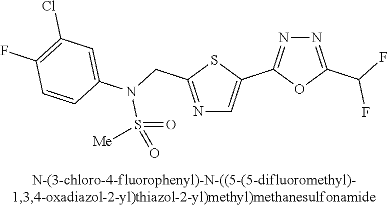 [SMILES: CS(=O)(=O)N(Cc1ncc(-c2nnc(C(F)F)o2)s1)c1ccc(F)c(Cl)c1] |
| I-14 | 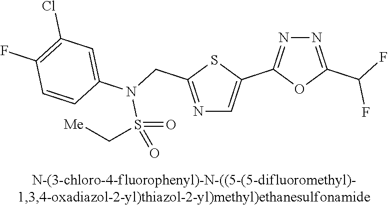 [SMILES: CCS(=O)(=O)N(Cc1ncc(-c2nnc(C(F)F)o2)s1)c1ccc(F)c(Cl)c1] |
| I-15 | 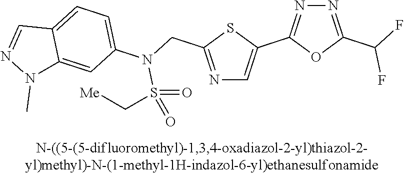 [SMILES: CCS(=O)(=O)N(Cc1ncc(-c2nnc(C(F)F)o2)s1)c1ccc2cnn(C)c2c1] |
| I-16 | 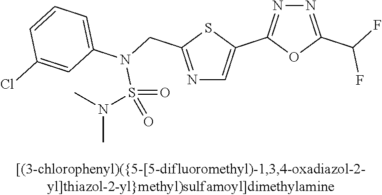 [SMILES: CN(C)S(=O)(=O)N(Cc1ncc(-c2nnc(C(F)F)o2)s1)c1cccc(Cl)c1] |
| I-17 | 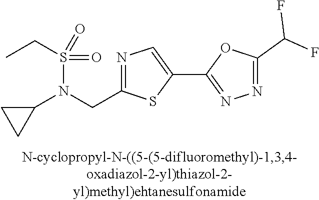 [SMILES: CCS(=O)(=O)N(Cc1ncc(-c2nnc(C(F)F)o2)s1)C1CC1] |
| I-18 | 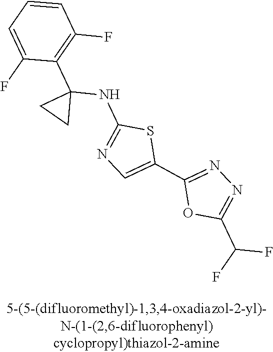 [SMILES: Fc1cccc(F)c1C1(Nc2ncc(-c3nnc(C(F)F)o3)s2)CC1] |
| I-19 | 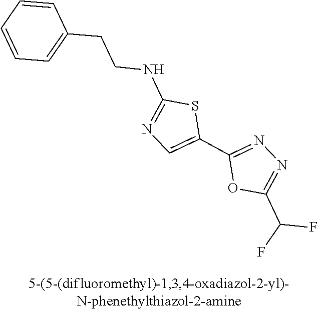 [SMILES: FC(F)c1nnc(-c2cnc(NCCc3ccccc3)s2)o1] |
| I-20 | 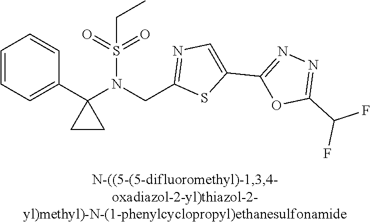 [SMILES: CCS(=O)(=O)N(Cc1ncc(-c2nnc(C(F)F)o2)s1)C1(c2ccccc2)CC1] |
| I-21 | 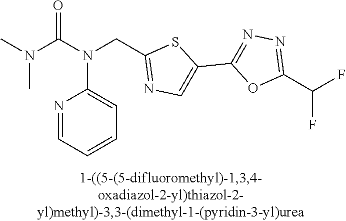 [SMILES: CN(C)C(=O)N(Cc1ncc(-c2nnc(C(F)F)o2)s1)c1ccccn1] |
| I-22 | 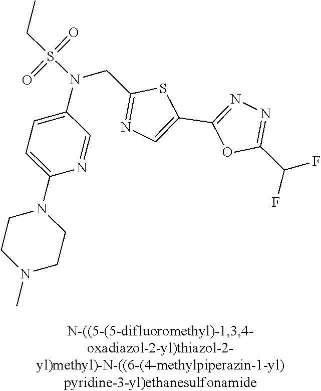 [SMILES: CCS(=O)(=O)N(Cc1ncc(-c2nnc(C(F)F)o2)s1)c1ccc(N2CCN(C)CC2)nc1] |
| I-23 | 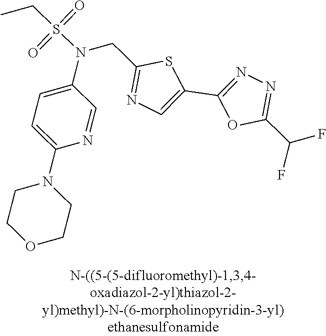 [SMILES: CCS(=O)(=O)N(Cc1ncc(-c2nnc(C(F)F)o2)s1)c1ccc(N2CCOCC2)nc1] |
| I-24 | 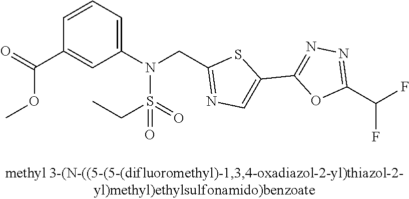 [SMILES: CCS(=O)(=O)N(Cc1ncc(-c2nnc(C(F)F)o2)s1)c1cccc(C(=O)OC)c1] |
| I-25 | 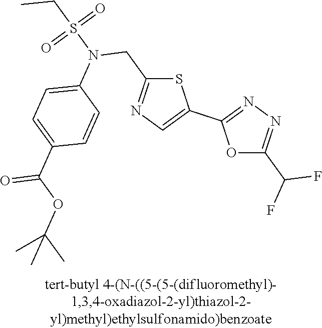 [SMILES: CCS(=O)(=O)N(Cc1ncc(-c2nnc(C(F)F)o2)s1)c1ccc(C(=O)OC(C)(C)C)cc1] |
| I-26 | 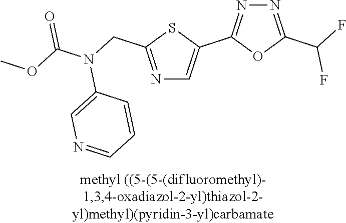 [SMILES: COC(=O)N(Cc1ncc(-c2nnc(C(F)F)o2)s1)c1cccnc1] |
| I-27 | 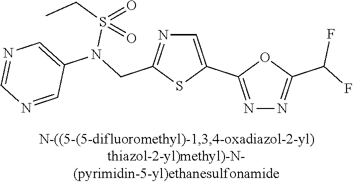 [SMILES: CCS(=O)(=O)N(Cc1ncc(-c2nnc(C(F)F)o2)s1)c1cncnc1] |
| I-28 | 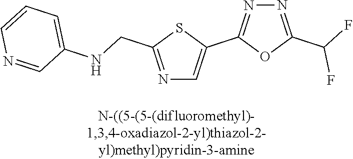 [SMILES: FC(F)c1nnc(-c2cnc(CNc3cccnc3)s2)o1] |
| I-29 | 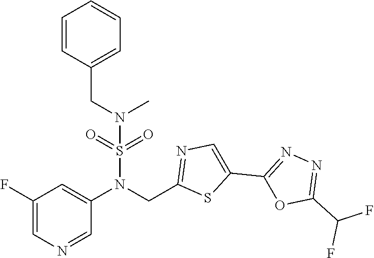 [SMILES: CN(Cc1ccccc1)S(=O)(=O)N(Cc1ncc(-c2nnc(C(F)F)o2)s1)c1cncc(F)c1] |
| I-30 | 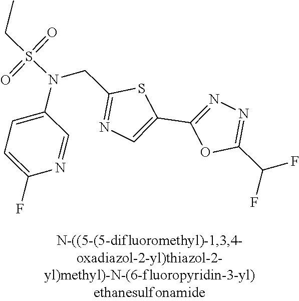 [SMILES: CCS(=O)(=O)N(Cc1ncc(-c2nnc(C(F)F)o2)s1)c1ccc(F)nc1] |
| I-31 | 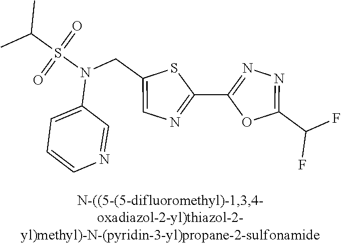 [SMILES: CC(C)S(=O)(=O)N(Cc1cnc(-c2nnc(C(F)F)o2)s1)c1cccnc1] |
| I-32 | 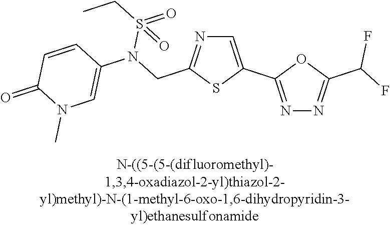 [SMILES: CCS(=O)(=O)N(Cc1ncc(-c2nnc(C(F)F)o2)s1)c1ccc(=O)n(C)c1] |
| I-33 | 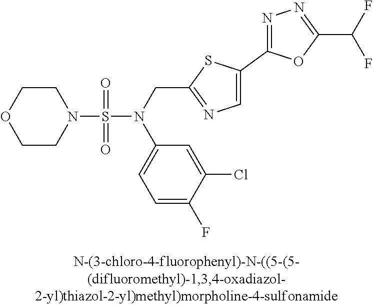 [SMILES: O=S(=O)(N1CCOCC1)N(Cc1ncc(-c2nnc(C(F)F)o2)s1)c1ccc(F)c(Cl)c1] |
| I-34 | 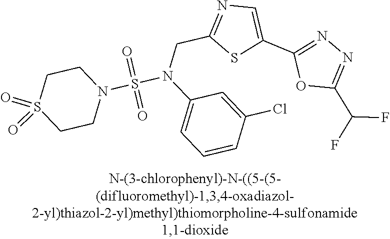 [SMILES: O=S1(=O)CCN(S(=O)(=O)N(Cc2ncc(-c3nnc(C(F)F)o3)s2)c2cccc(Cl)c2)CC1] |
| I-35 | 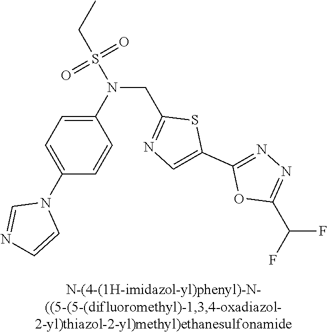 [SMILES: CCS(=O)(=O)N(Cc1ncc(-c2nnc(C(F)F)o2)s1)c1ccc(-n2ccnc2)cc1] |
| I-36 | 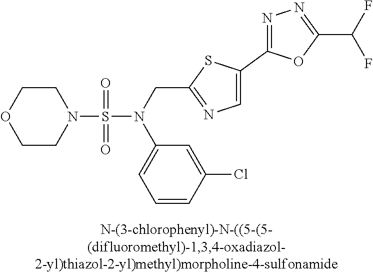 [SMILES: O=S(=O)(N1CCOCC1)N(Cc1ncc(-c2nnc(C(F)F)o2)s1)c1cccc(Cl)c1] |
| I-37 | 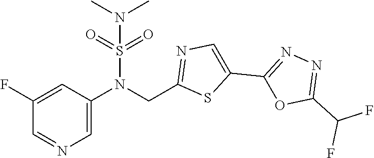 [SMILES: CN(C)S(=O)(=O)N(Cc1ncc(-c2nnc(C(F)F)o2)s1)c1cncc(F)c1] |
| I-38 | 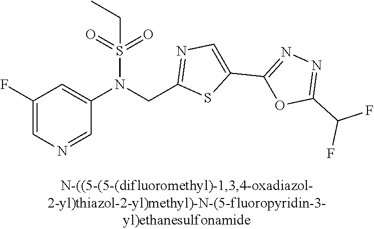 [SMILES: CCS(=O)(=O)N(Cc1ncc(-c2nnc(C(F)F)o2)s1)c1cncc(F)c1] |
| I-39 | 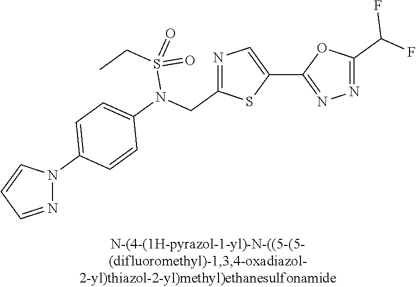 [SMILES: CCS(=O)(=O)N(Cc1ncc(-c2nnc(C(F)F)o2)s1)c1ccc(-n2cccn2)cc1] |
| I-40 | 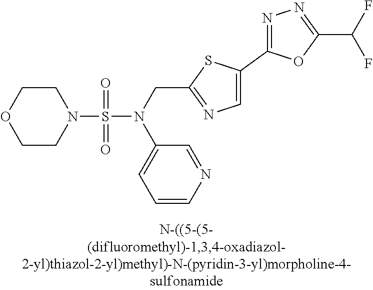 [SMILES: O=S(=O)(N1CCOCC1)N(Cc1ncc(-c2nnc(C(F)F)o2)s1)c1cccnc1] |
| I-43 | 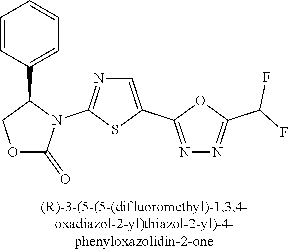 [SMILES: O=C1OC[C@@H](c2ccccc2)N1c1ncc(-c2nnc(C(F)F)o2)s1] |
| I-44 | 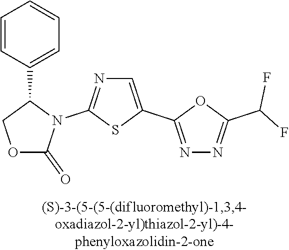 [SMILES: O=C1OC[C@H](c2ccccc2)N1c1ncc(-c2nnc(C(F)F)o2)s1] |
| I-45 | 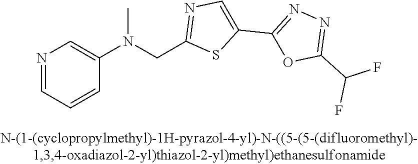 [SMILES: CN(Cc1ncc(-c2nnc(C(F)F)o2)s1)c1cccnc1] |
| I-46 | 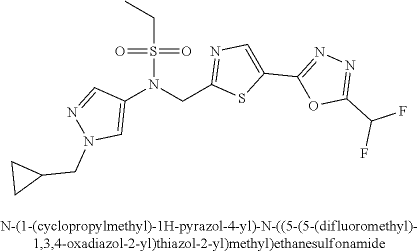 [SMILES: CCS(=O)(=O)N(Cc1ncc(-c2nnc(C(F)F)o2)s1)c1cnn(CC2CC2)c1] |
| I-47 | 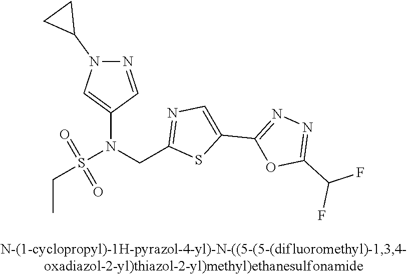 [SMILES: CCS(=O)(=O)N(Cc1ncc(-c2nnc(C(F)F)o2)s1)c1cnn(C2CC2)c1] |
| I-48 | 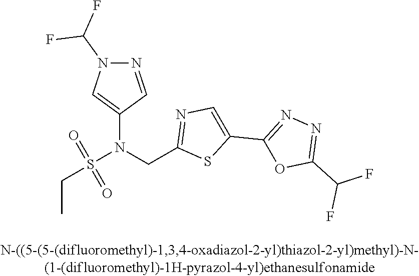 [SMILES: CCS(=O)(=O)N(Cc1ncc(-c2nnc(C(F)F)o2)s1)c1cnn(C(F)F)c1] |
| I-49 | 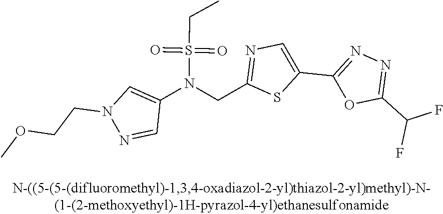 [SMILES: CCS(=O)(=O)N(Cc1ncc(-c2nnc(C(F)F)o2)s1)c1cnn(CCOC)c1] |
| I-50 | 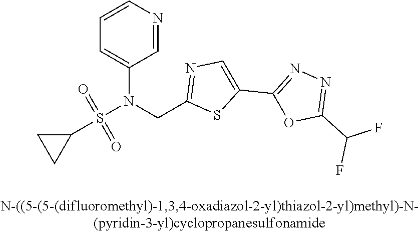 [SMILES: O=S(=O)(C1CC1)N(Cc1ncc(-c2nnc(C(F)F)o2)s1)c1cccnc1] |
| I-51 | 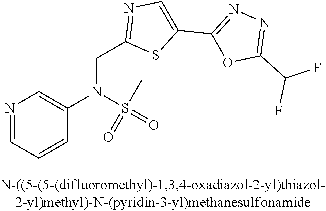 [SMILES: CS(=O)(=O)N(Cc1ncc(-c2nnc(C(F)F)o2)s1)c1cccnc1] |
| I-52 | 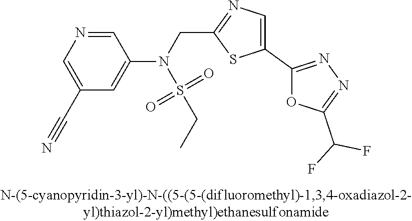 [SMILES: CCS(=O)(=O)N(Cc1ncc(-c2nnc(C(F)F)o2)s1)c1cncc(C#N)c1] |
| I-53 | 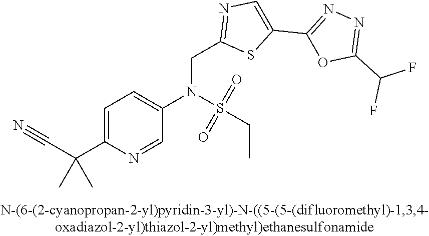 [SMILES: CCS(=O)(=O)N(Cc1ncc(-c2nnc(C(F)F)o2)s1)c1ccc(C(C)(C)C#N)nc1] |
| I-54 | 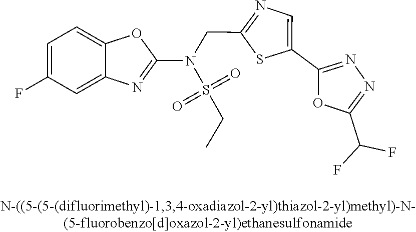 [SMILES: CCS(=O)(=O)N(Cc1ncc(-c2nnc(C(F)F)o2)s1)c1nc2cc(F)ccc2o1] |
| I-55 | 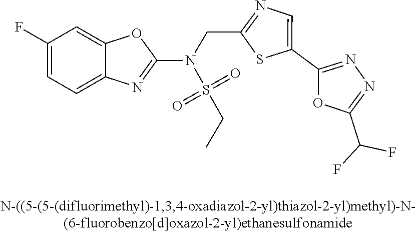 [SMILES: CCS(=O)(=O)N(Cc1ncc(-c2nnc(C(F)F)o2)s1)c1nc2ccc(F)cc2o1] |
| I-56 | 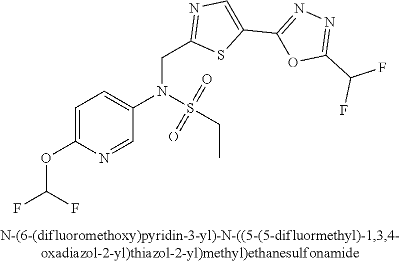 [SMILES: CCS(=O)(=O)N(Cc1ncc(-c2nnc(C(F)F)o2)s1)c1ccc(OC(F)F)nc1] |
| I-57 | 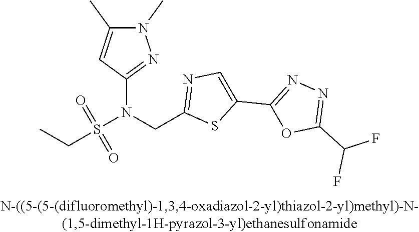 [SMILES: CCS(=O)(=O)N(Cc1ncc(-c2nnc(C(F)F)o2)s1)c1cc(C)n(C)n1] |
| I-58 | 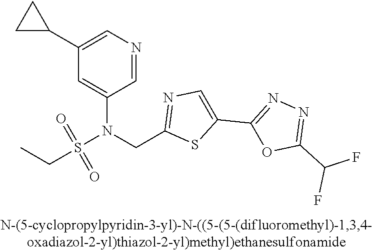 [SMILES: CCS(=O)(=O)N(Cc1ncc(-c2nnc(C(F)F)o2)s1)c1cncc(C2CC2)c1] |
| I-59 | 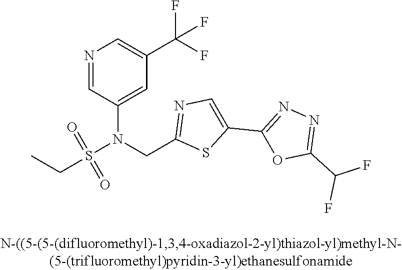 [SMILES: CCS(=O)(=O)N(Cc1ncc(-c2nnc(C(F)F)o2)s1)c1cncc(C(F)(F)F)c1] |
| I-60 | 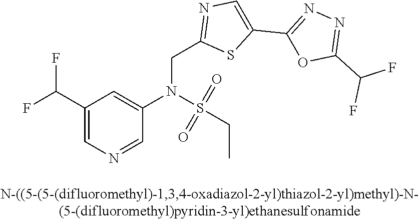 [SMILES: CCS(=O)(=O)N(Cc1ncc(-c2nnc(C(F)F)o2)s1)c1cncc(C(F)F)c1] |
| I-61 | 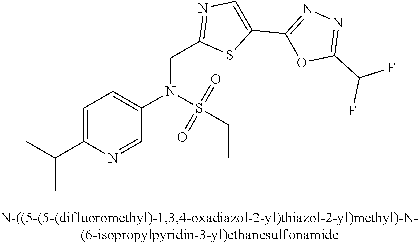 [SMILES: CCS(=O)(=O)N(Cc1ncc(-c2nnc(C(F)F)o2)s1)c1ccc(C(C)C)nc1] |
| I-62 | 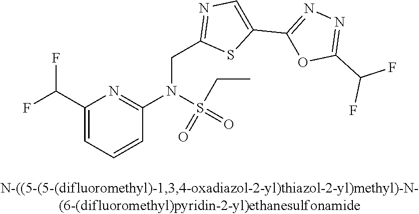 [SMILES: CCS(=O)(=O)N(Cc1ncc(-c2nnc(C(F)F)o2)s1)c1cccc(C(F)F)n1] |
| I-63 | 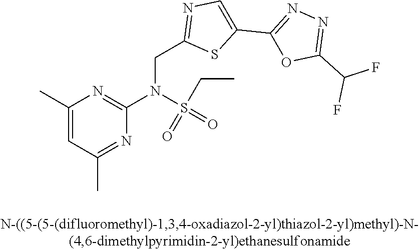 [SMILES: CCS(=O)(=O)N(Cc1ncc(-c2nnc(C(F)F)o2)s1)c1nc(C)cc(C)n1] |
| I-64 | 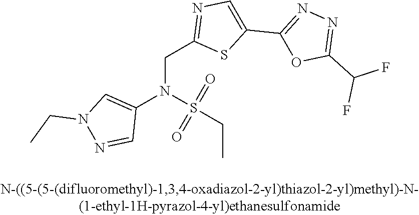 [SMILES: CCn1cc(N(Cc2ncc(-c3nnc(C(F)F)o3)s2)S(=O)(=O)CC)cn1] |
| I-65 | 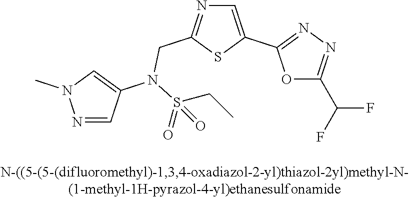 [SMILES: CCS(=O)(=O)N(Cc1ncc(-c2nnc(C(F)F)o2)s1)c1cnn(C)c1] |
| I-66 | 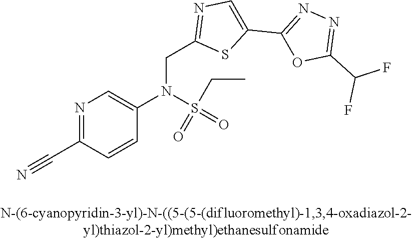 [SMILES: CCS(=O)(=O)N(Cc1ncc(-c2nnc(C(F)F)o2)s1)c1ccc(C#N)nc1] |
| I-67 | 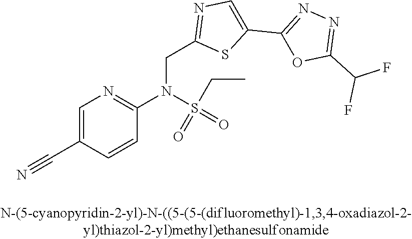 [SMILES: CCS(=O)(=O)N(Cc1ncc(-c2nnc(C(F)F)o2)s1)c1ccc(C#N)cn1] |
| I-68 | 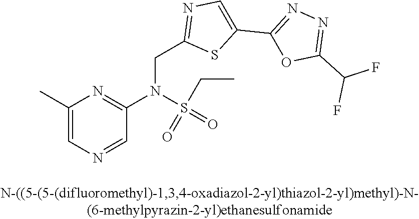 [SMILES: CCS(=O)(=O)N(Cc1ncc(-c2nnc(C(F)F)o2)s1)c1cncc(C)n1] |
| I-69 | 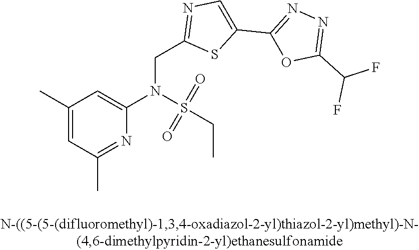 [SMILES: CCS(=O)(=O)N(Cc1ncc(-c2nnc(C(F)F)o2)s1)c1cc(C)cc(C)n1] |
| I-70 | 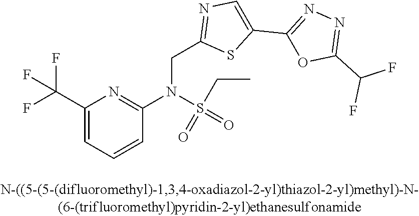 [SMILES: CCS(=O)(=O)N(Cc1ncc(-c2nnc(C(F)F)o2)s1)c1cccc(C(F)(F)F)n1] |
| I-71 | 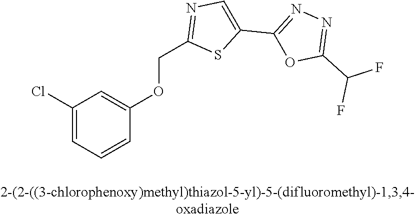 [SMILES: FC(F)c1nnc(-c2cnc(COc3cccc(Cl)c3)s2)o1] |
| I-72 | 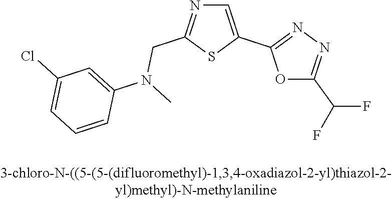 [SMILES: CN(Cc1ncc(-c2nnc(C(F)F)o2)s1)c1cccc(Cl)c1] |
| I-73 | 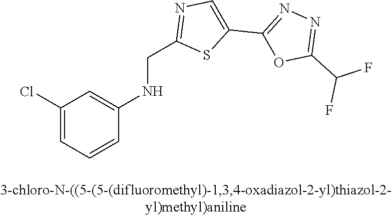 [SMILES: FC(F)c1nnc(-c2cnc(CNc3cccc(Cl)c3)s2)o1] |
| I-74 | 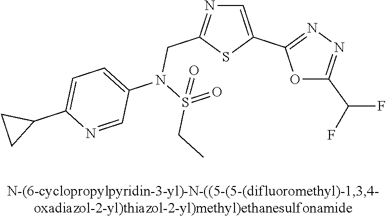 [SMILES: CCS(=O)(=O)N(Cc1ncc(-c2nnc(C(F)F)o2)s1)c1ccc(C2CC2)nc1] |
| I-75 | 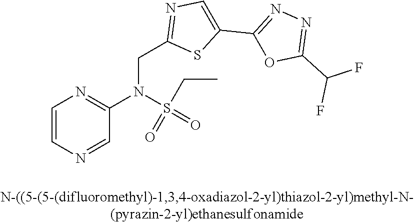 [SMILES: CCS(=O)(=O)N(Cc1ncc(-c2nnc(C(F)F)o2)s1)c1cnccn1] |
| I-76 | 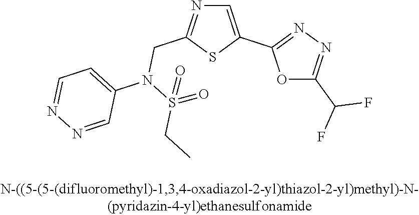 [SMILES: CCS(=O)(=O)N(Cc1ncc(-c2nnc(C(F)F)o2)s1)c1ccnnc1] |
| I-77 | 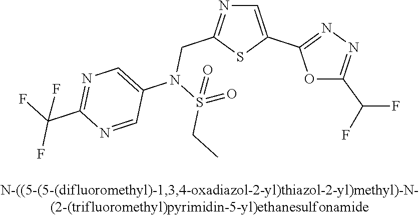 [SMILES: CCS(=O)(=O)N(Cc1ncc(-c2nnc(C(F)F)o2)s1)c1cnc(C(F)(F)F)nc1] |
| I-78 | 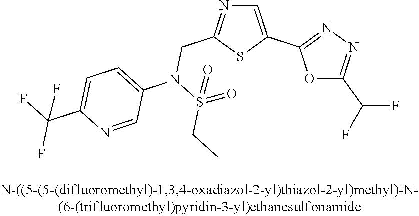 [SMILES: CCS(=O)(=O)N(Cc1ncc(-c2nnc(C(F)F)o2)s1)c1ccc(C(F)(F)F)nc1] |
| I-79 | 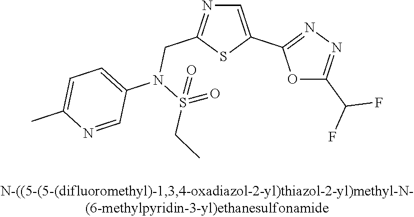 [SMILES: CCS(=O)(=O)N(Cc1ncc(-c2nnc(C(F)F)o2)s1)c1ccc(C)nc1] |
| I-80 | 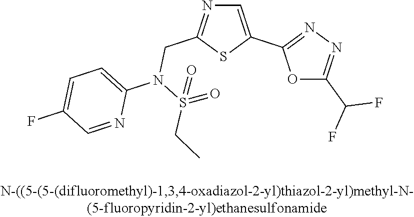 [SMILES: CCS(=O)(=O)N(Cc1ncc(-c2nnc(C(F)F)o2)s1)c1ccc(F)cn1] |
| I-81 | 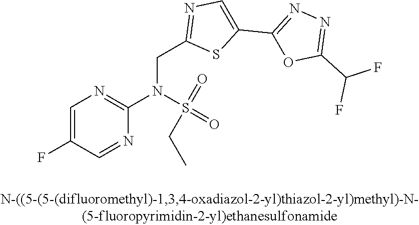 [SMILES: CCS(=O)(=O)N(Cc1ncc(-c2nnc(C(F)F)o2)s1)c1ncc(F)cn1] |
| I-82 | 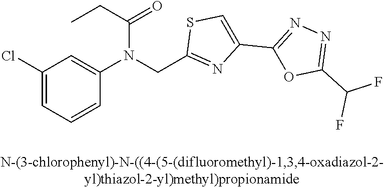 [SMILES: CCC(=O)N(Cc1nc(-c2nnc(C(F)F)o2)cs1)c1cccc(Cl)c1] |
| I-83 | 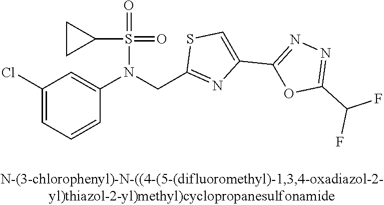 [SMILES: O=S(=O)(C1CC1)N(Cc1nc(-c2nnc(C(F)F)o2)cs1)c1cccc(Cl)c1] |
| I-84 | 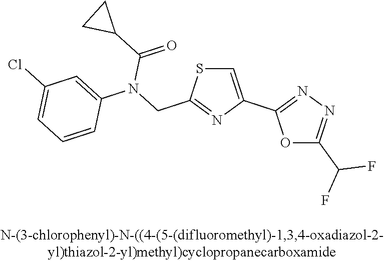 [SMILES: O=C(C1CC1)N(Cc1nc(-c2nnc(C(F)F)o2)cs1)c1cccc(Cl)c1] |
| I-85 | 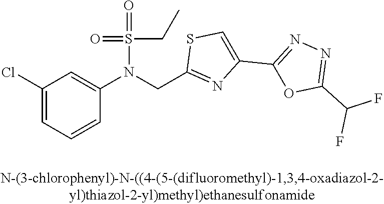 [SMILES: CCS(=O)(=O)N(Cc1nc(-c2nnc(C(F)F)o2)cs1)c1cccc(Cl)c1] |
| I-86 | 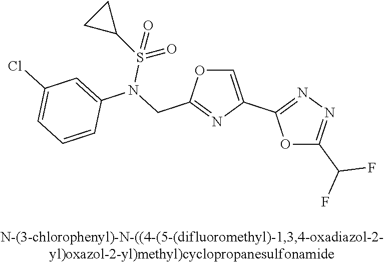 [SMILES: O=S(=O)(C1CC1)N(Cc1nc(-c2nnc(C(F)F)o2)co1)c1cccc(Cl)c1] |
| I-87 | 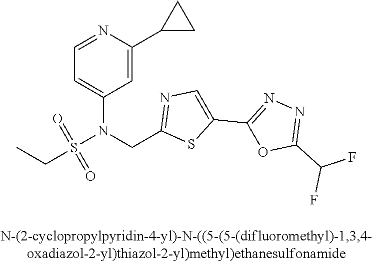 [SMILES: CCS(=O)(=O)N(Cc1ncc(-c2nnc(C(F)F)o2)s1)c1ccnc(C2CC2)c1] |
| I-88 | 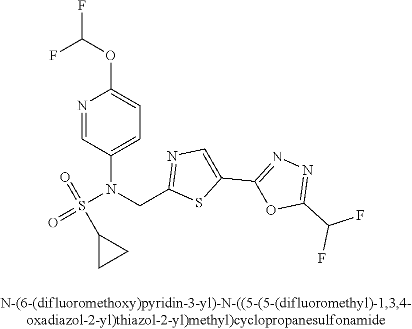 [SMILES: O=S(=O)(C1CC1)N(Cc1ncc(-c2nnc(C(F)F)o2)s1)c1ccc(OC(F)F)nc1] |
| I-89 | 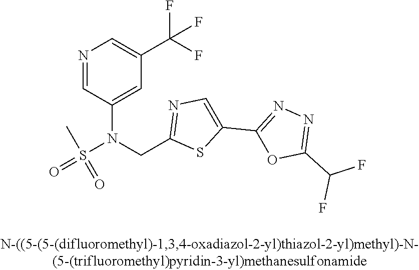 [SMILES: CS(=O)(=O)N(Cc1ncc(-c2nnc(C(F)F)o2)s1)c1cncc(C(F)(F)F)c1] |
| I-90 | 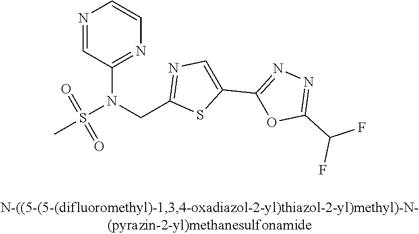 [SMILES: CS(=O)(=O)N(Cc1ncc(-c2nnc(C(F)F)o2)s1)c1cnccn1] |
| I-91 | 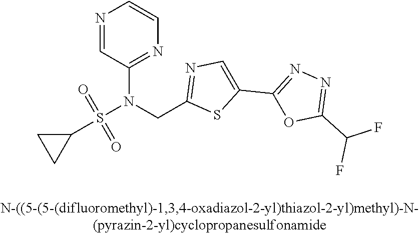 [SMILES: O=S(=O)(C1CC1)N(Cc1ncc(-c2nnc(C(F)F)o2)s1)c1cnccn1] |
| I-92 | 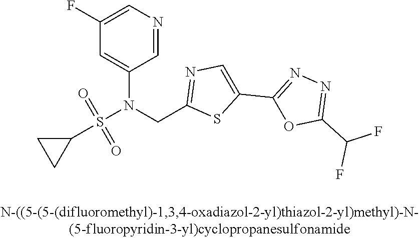 [SMILES: O=S(=O)(C1CC1)N(Cc1ncc(-c2nnc(C(F)F)o2)s1)c1cncc(F)c1] |
| I-93 | 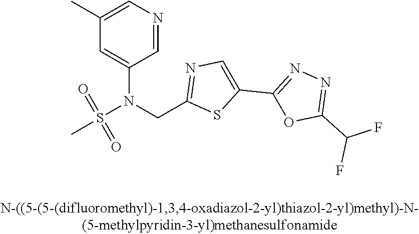 [SMILES: Cc1cncc(N(Cc2ncc(-c3nnc(C(F)F)o3)s2)S(C)(=O)=O)c1] |
| I-94 | 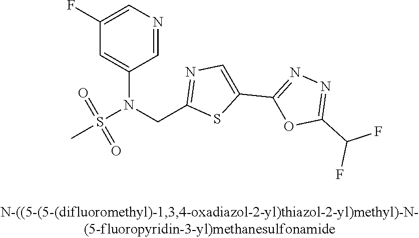 [SMILES: CS(=O)(=O)N(Cc1ncc(-c2nnc(C(F)F)o2)s1)c1cncc(F)c1] |
| I-95 | 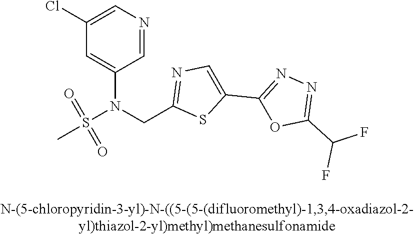 [SMILES: CS(=O)(=O)N(Cc1ncc(-c2nnc(C(F)F)o2)s1)c1cncc(Cl)c1] |
| I-96 | 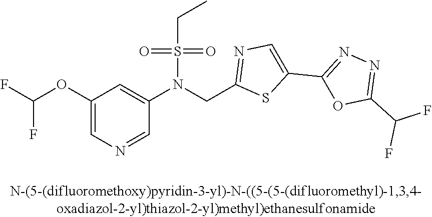 [SMILES: CCS(=O)(=O)N(Cc1ncc(-c2nnc(C(F)F)o2)s1)c1cncc(OC(F)F)c1] |
| I-97 | 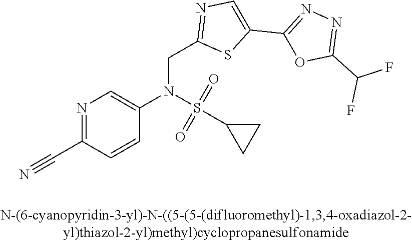 [SMILES: N#Cc1ccc(N(Cc2ncc(-c3nnc(C(F)F)o3)s2)S(=O)(=O)C2CC2)cn1] |
| I-98 | 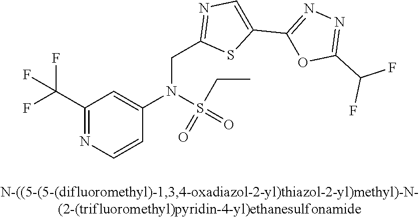 [SMILES: CCS(=O)(=O)N(Cc1ncc(-c2nnc(C(F)F)o2)s1)c1ccnc(C(F)(F)F)c1] |
| I-99 | 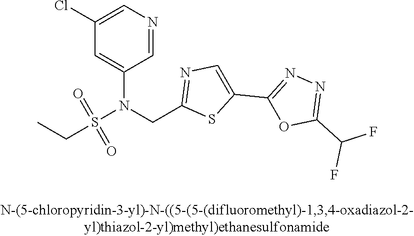 [SMILES: CCS(=O)(=O)N(Cc1ncc(-c2nnc(C(F)F)o2)s1)c1cncc(Cl)c1] |
| I-100 | 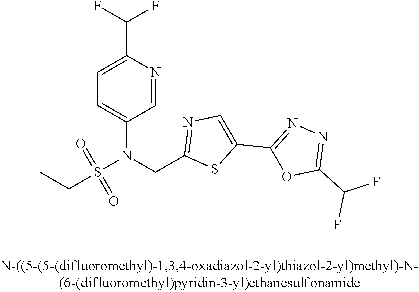 [SMILES: CCS(=O)(=O)N(Cc1ncc(-c2nnc(C(F)F)o2)s1)c1ccc(C(F)F)nc1] |
| I-101 | 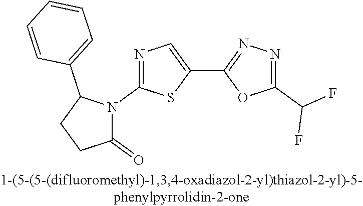 [SMILES: O=C1CCC(c2ccccc2)N1c1ncc(-c2nnc(C(F)F)o2)s1] |
| I-102 | 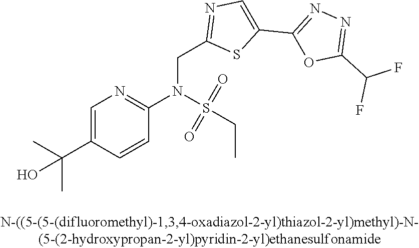 [SMILES: CCS(=O)(=O)N(Cc1ncc(-c2nnc(C(F)F)o2)s1)c1ccc(C(C)(C)O)cn1] |
| I-103 | 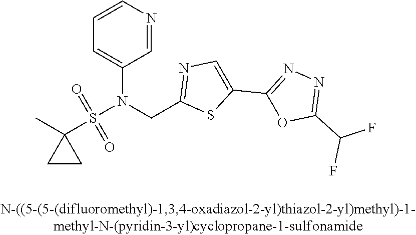 [SMILES: CC1(S(=O)(=O)N(Cc2ncc(-c3nnc(C(F)F)o3)s2)c2cccnc2)CC1] |
| I-104 | 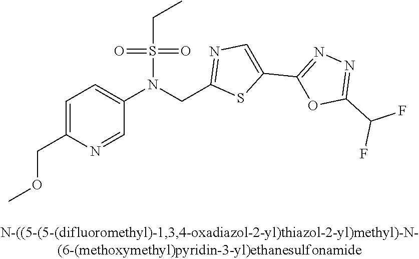 [SMILES: CCS(=O)(=O)N(Cc1ncc(-c2nnc(C(F)F)o2)s1)c1ccc(COC)nc1] |
| I-105 | 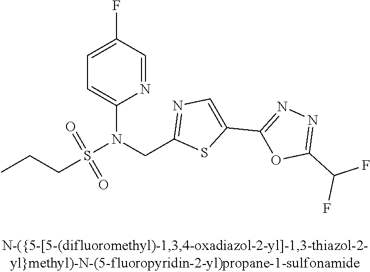 [SMILES: CCCS(=O)(=O)N(Cc1ncc(-c2nnc(C(F)F)o2)s1)c1ccc(F)cn1] |
| I-106 | 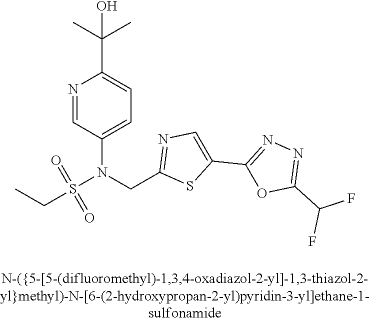 [SMILES: CCS(=O)(=O)N(Cc1ncc(-c2nnc(C(F)F)o2)s1)c1ccc(C(C)(C)O)nc1] |
| I-107 | 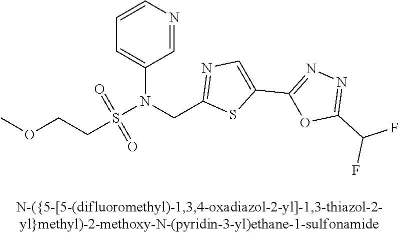 [SMILES: COCCS(=O)(=O)N(Cc1ncc(-c2nnc(C(F)F)o2)s1)c1cccnc1] |
| I-108 | 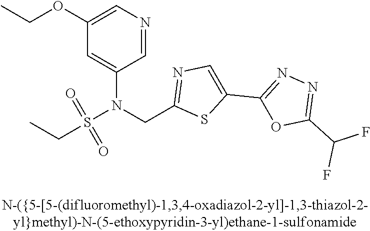 [SMILES: CCOc1cncc(N(Cc2ncc(-c3nnc(C(F)F)o3)s2)S(=O)(=O)CC)c1] |
| I-109 | 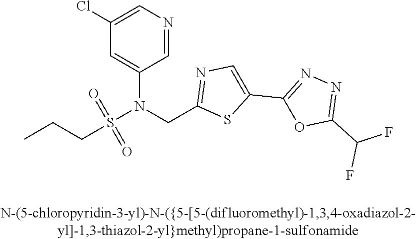 [SMILES: CCCS(=O)(=O)N(Cc1ncc(-c2nnc(C(F)F)o2)s1)c1cncc(Cl)c1] |
| I-110 | 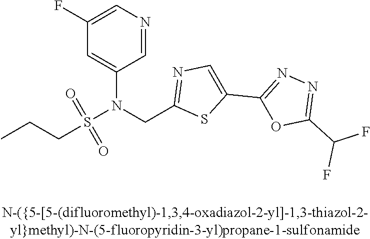 [SMILES: CCCS(=O)(=O)N(Cc1ncc(-c2nnc(C(F)F)o2)s1)c1cncc(F)c1] |
| I-111 | 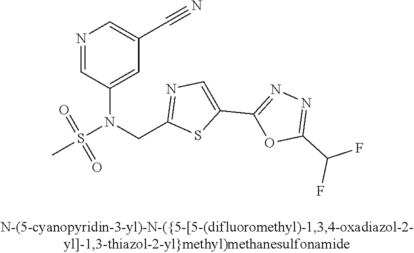 [SMILES: CS(=O)(=O)N(Cc1ncc(-c2nnc(C(F)F)o2)s1)c1cncc(C#N)c1] |
| I-112 | 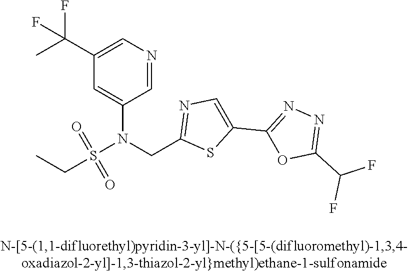 [SMILES: CCS(=O)(=O)N(Cc1ncc(-c2nnc(C(F)F)o2)s1)c1cncc(C(C)(F)F)c1] |
| I-113 | 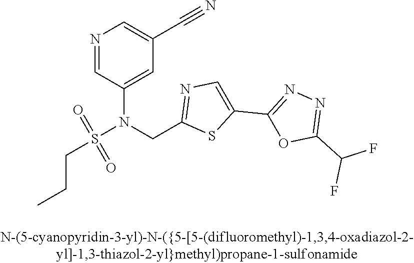 [SMILES: CCCS(=O)(=O)N(Cc1ncc(-c2nnc(C(F)F)o2)s1)c1cncc(C#N)c1] |
| I-114 | 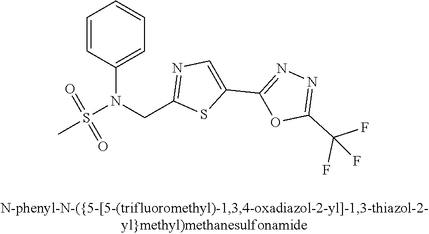 [SMILES: CS(=O)(=O)N(Cc1ncc(-c2nnc(C(F)(F)F)o2)s1)c1ccccc1] |
| I-115 | 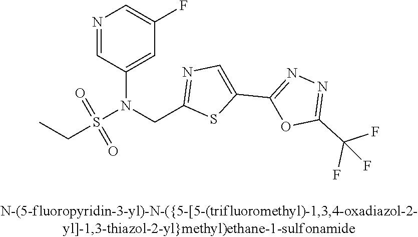 [SMILES: CCS(=O)(=O)N(Cc1ncc(-c2nnc(C(F)(F)F)o2)s1)c1cncc(F)c1] |
| I-116 | 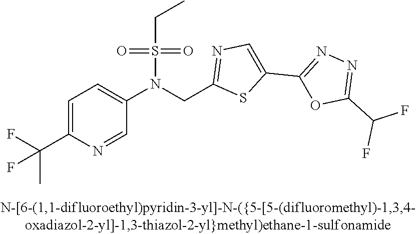 [SMILES: CCS(=O)(=O)N(Cc1ncc(-c2nnc(C(F)F)o2)s1)c1ccc(C(C)(F)F)nc1] |
| I-117 | 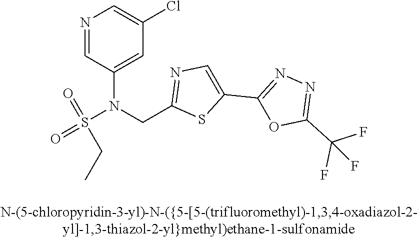 [SMILES: CCS(=O)(=O)N(Cc1ncc(-c2nnc(C(F)(F)F)o2)s1)c1cncc(Cl)c1] |
| I-118 | 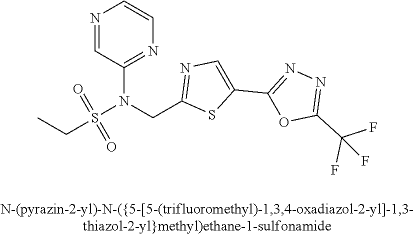 [SMILES: CCS(=O)(=O)N(Cc1ncc(-c2nnc(C(F)(F)F)o2)s1)c1cnccn1] |
| I-119 | 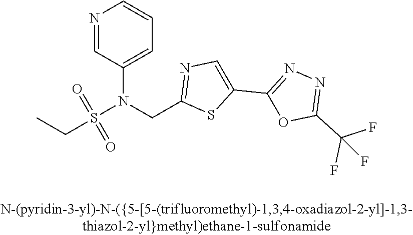 [SMILES: CCS(=O)(=O)N(Cc1ncc(-c2nnc(C(F)(F)F)o2)s1)c1cccnc1] |
| I-120 | 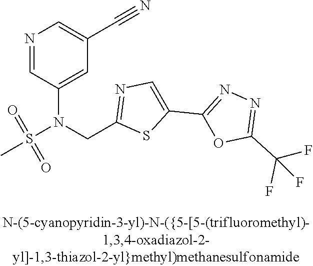 [SMILES: CS(=O)(=O)N(Cc1ncc(-c2nnc(C(F)(F)F)o2)s1)c1cncc(C#N)c1] |
| I-121 | 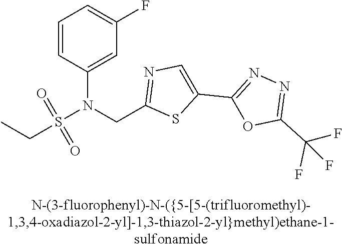 [SMILES: CCS(=O)(=O)N(Cc1ncc(-c2nnc(C(F)(F)F)o2)s1)c1cccc(F)c1] |
| I-122 | 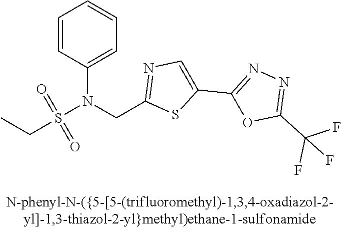 [SMILES: CCS(=O)(=O)N(Cc1ncc(-c2nnc(C(F)(F)F)o2)s1)c1ccccc1] |
| I-123 | 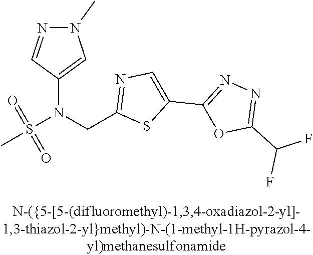 [SMILES: Cn1cc(N(Cc2ncc(-c3nnc(C(F)F)o3)s2)S(C)(=O)=O)cn1] |
| I-124 | 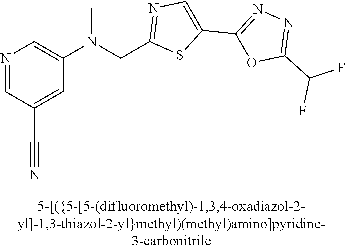 [SMILES: CN(Cc1ncc(-c2nnc(C(F)F)o2)s1)c1cncc(C#N)c1] |
| I-125 | 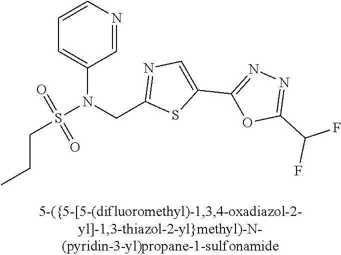 [SMILES: CCCS(=O)(=O)N(Cc1ncc(-c2nnc(C(F)F)o2)s1)c1cccnc1] |
| I-126 | 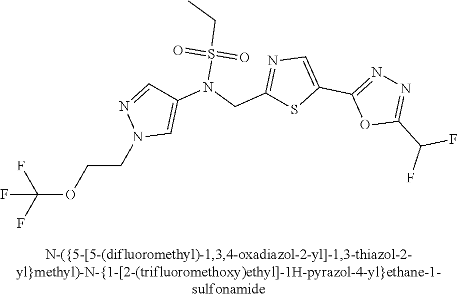 [SMILES: CCS(=O)(=O)N(Cc1ncc(-c2nnc(C(F)F)o2)s1)c1cnn(CCOC(F)(F)F)c1] |
| I-127 | 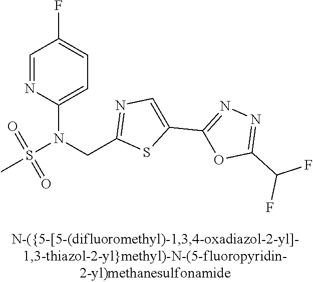 [SMILES: CS(=O)(=O)N(Cc1ncc(-c2nnc(C(F)F)o2)s1)c1ccc(F)cn1] |
| I-128 | 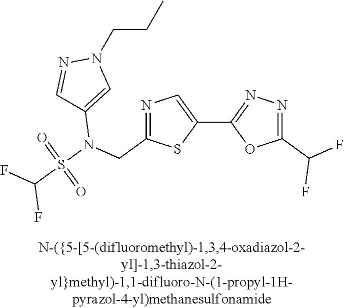 [SMILES: CCCn1cc(N(Cc2ncc(-c3nnc(C(F)F)o3)s2)S(=O)(=O)C(F)F)cn1] |
| I-129 | 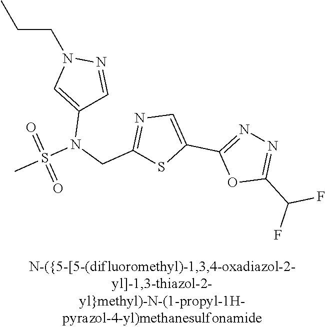 [SMILES: CCCn1cc(N(Cc2ncc(-c3nnc(C(F)F)o3)s2)S(C)(=O)=O)cn1] |
| I-130 | 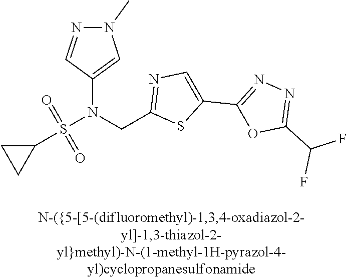 [SMILES: Cn1cc(N(Cc2ncc(-c3nnc(C(F)F)o3)s2)S(=O)(=O)C2CC2)cn1] |
| I-131 | 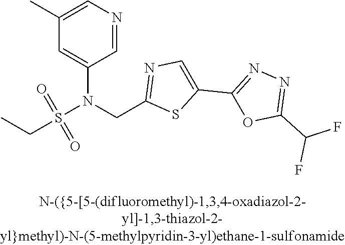 [SMILES: CCS(=O)(=O)N(Cc1ncc(-c2nnc(C(F)F)o2)s1)c1cncc(C)c1] |
| I-132 | 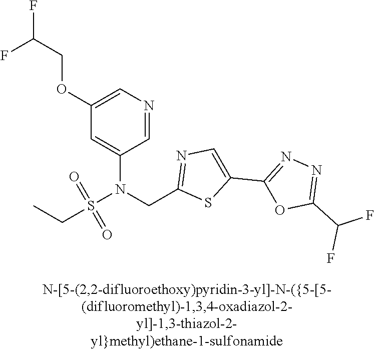 [SMILES: CCS(=O)(=O)N(Cc1ncc(-c2nnc(C(F)F)o2)s1)c1cncc(OCC(F)F)c1] |
| I-133 | 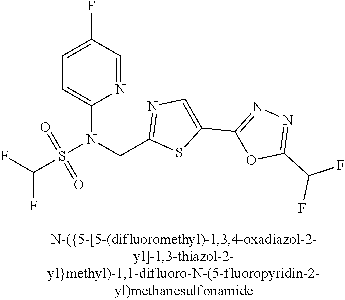 [SMILES: O=S(=O)(C(F)F)N(Cc1ncc(-c2nnc(C(F)F)o2)s1)c1ccc(F)cn1] |
| I-134 | 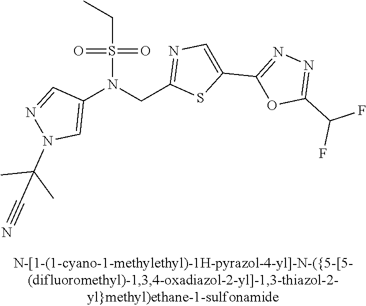 [SMILES: CCS(=O)(=O)N(Cc1ncc(-c2nnc(C(F)F)o2)s1)c1cnn(C(C)(C)C#N)c1] |
| I-135 | 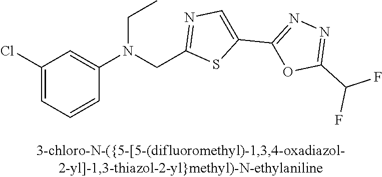 [SMILES: CCN(Cc1ncc(-c2nnc(C(F)F)o2)s1)c1cccc(Cl)c1] |
| I-136 | 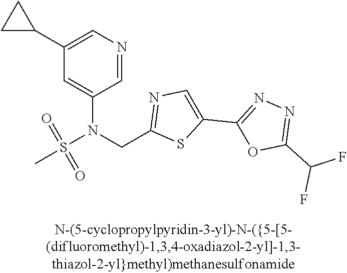 [SMILES: CS(=O)(=O)N(Cc1ncc(-c2nnc(C(F)F)o2)s1)c1cncc(C2CC2)c1] |
| I-137 | 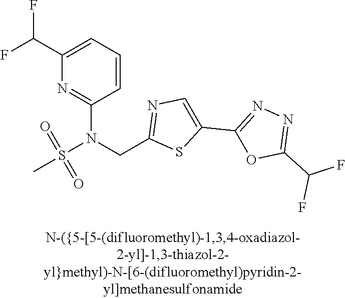 [SMILES: CS(=O)(=O)N(Cc1ncc(-c2nnc(C(F)F)o2)s1)c1cccc(C(F)F)n1] |
| I-138 | 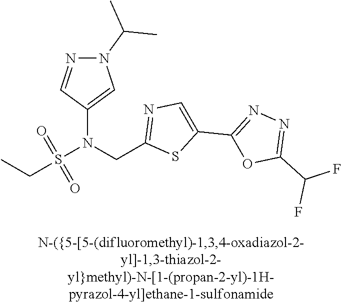 [SMILES: CCS(=O)(=O)N(Cc1ncc(-c2nnc(C(F)F)o2)s1)c1cnn(C(C)C)c1] |
| I-139 | 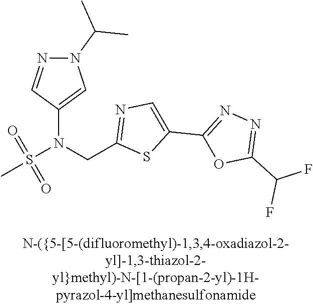 [SMILES: CC(C)n1cc(N(Cc2ncc(-c3nnc(C(F)F)o3)s2)S(C)(=O)=O)cn1] |
| I-140 | 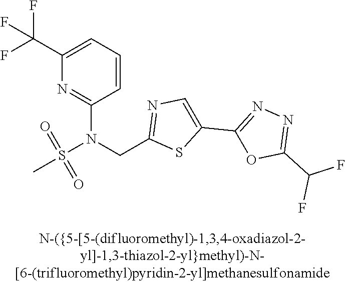 [SMILES: CS(=O)(=O)N(Cc1ncc(-c2nnc(C(F)F)o2)s1)c1cccc(C(F)(F)F)n1] |
| I-141 | 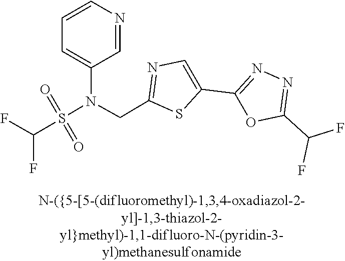 [SMILES: O=S(=O)(C(F)F)N(Cc1ncc(-c2nnc(C(F)F)o2)s1)c1cccnc1] |
| I-142 | 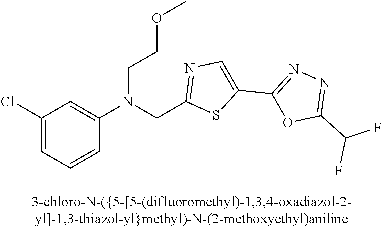 [SMILES: COCCN(Cc1ncc(-c2nnc(C(F)F)o2)s1)c1cccc(Cl)c1] |
| I-143 | 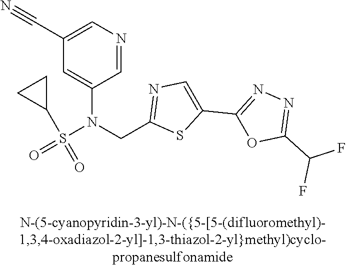 [SMILES: N#Cc1cncc(N(Cc2ncc(-c3nnc(C(F)F)o3)s2)S(=O)(=O)C2CC2)c1] |
| I-144 | 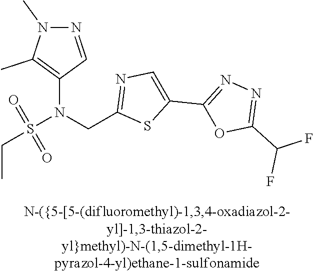 [SMILES: CCS(=O)(=O)N(Cc1ncc(-c2nnc(C(F)F)o2)s1)c1cnn(C)c1C] |
| I-145 | 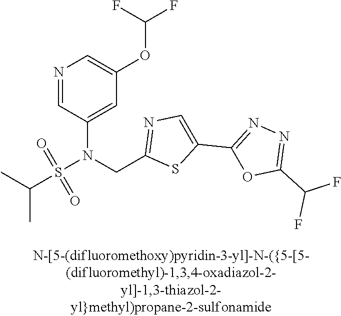 [SMILES: CC(C)S(=O)(=O)N(Cc1ncc(-c2nnc(C(F)F)o2)s1)c1cncc(OC(F)F)c1] |
| I-146 | 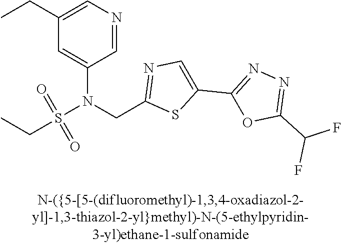 [SMILES: CCc1cncc(N(Cc2ncc(-c3nnc(C(F)F)o3)s2)S(=O)(=O)CC)c1] |
| I-147 | 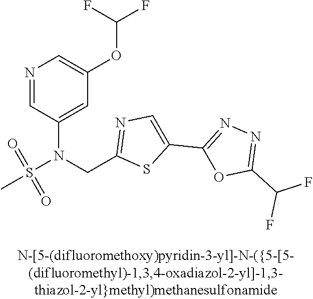 [SMILES: CS(=O)(=O)N(Cc1ncc(-c2nnc(C(F)F)o2)s1)c1cncc(OC(F)F)c1] |
| I-148 | 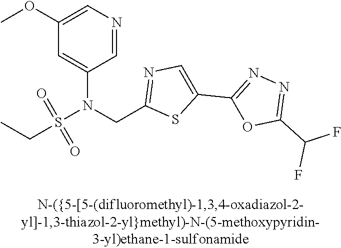 [SMILES: CCS(=O)(=O)N(Cc1ncc(-c2nnc(C(F)F)o2)s1)c1cncc(OC)c1] |
| I-149 | 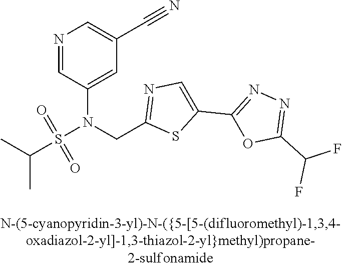 [SMILES: CC(C)S(=O)(=O)N(Cc1ncc(-c2nnc(C(F)F)o2)s1)c1cncc(C#N)c1] |
| I-150 | 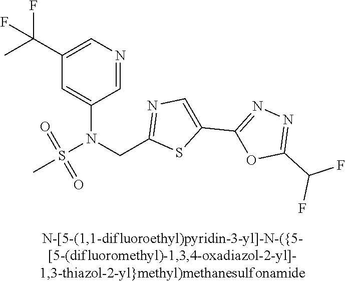 [SMILES: CC(F)(F)c1cncc(N(Cc2ncc(-c3nnc(C(F)F)o3)s2)S(C)(=O)=O)c1] |
| I-151 | 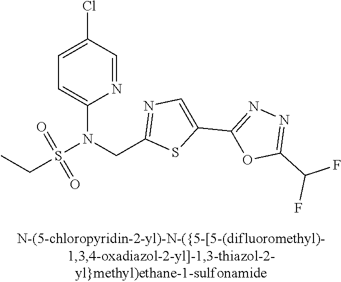 [SMILES: CCS(=O)(=O)N(Cc1ncc(-c2nnc(C(F)F)o2)s1)c1ccc(Cl)cn1] |
| I-152 | 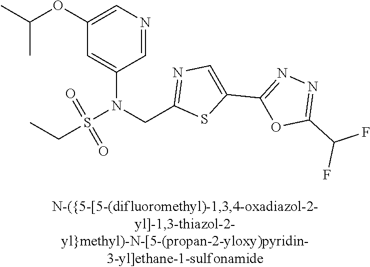 [SMILES: CCS(=O)(=O)N(Cc1ncc(-c2nnc(C(F)F)o2)s1)c1cncc(OC(C)C)c1] |
| I-153 | 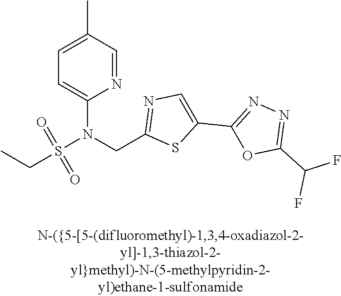 [SMILES: CCS(=O)(=O)N(Cc1ncc(-c2nnc(C(F)F)o2)s1)c1ccc(C)cn1] |
| I-155 | 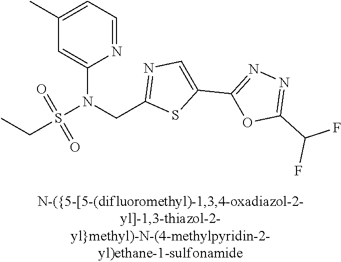 [SMILES: CCS(=O)(=O)N(Cc1ncc(-c2nnc(C(F)F)o2)s1)c1cc(C)ccn1] |
| I-156 | 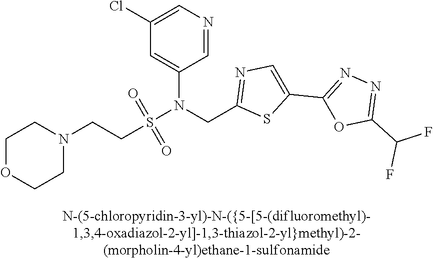 [SMILES: O=S(=O)(CCN1CCOCC1)N(Cc1ncc(-c2nnc(C(F)F)o2)s1)c1cncc(Cl)c1] |
| I-157 | 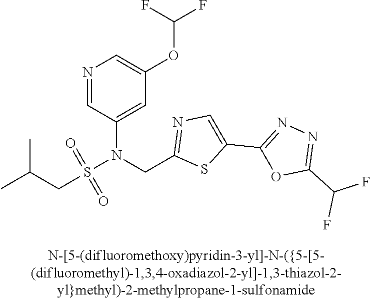 [SMILES: CC(C)CS(=O)(=O)N(Cc1ncc(-c2nnc(C(F)F)o2)s1)c1cncc(OC(F)F)c1] |
| I-158 | 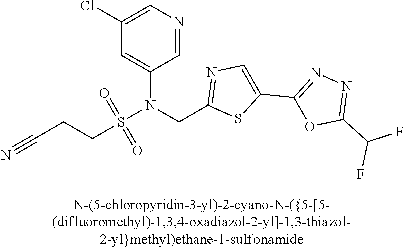 [SMILES: N#CCCS(=O)(=O)N(Cc1ncc(-c2nnc(C(F)F)o2)s1)c1cncc(Cl)c1] |
| I-159 | 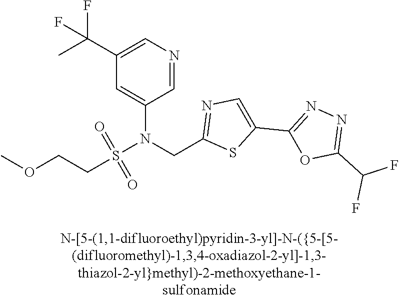 [SMILES: COCCS(=O)(=O)N(Cc1ncc(-c2nnc(C(F)F)o2)s1)c1cncc(C(C)(F)F)c1] |
| I-160 | 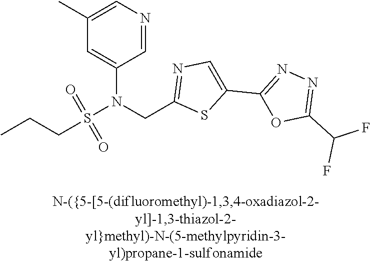 [SMILES: CCCS(=O)(=O)N(Cc1ncc(-c2nnc(C(F)F)o2)s1)c1cncc(C)c1] |
| I-161 | 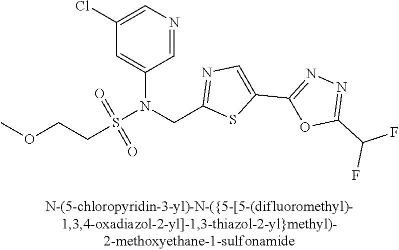 [SMILES: COCCS(=O)(=O)N(Cc1ncc(-c2nnc(C(F)F)o2)s1)c1cncc(Cl)c1] |
| I-162 | 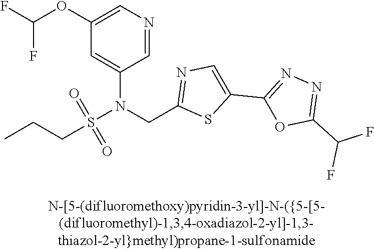 [SMILES: CCCS(=O)(=O)N(Cc1ncc(-c2nnc(C(F)F)o2)s1)c1cncc(OC(F)F)c1] |
| I-163 | 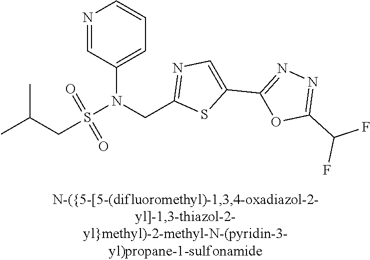 [SMILES: CC(C)CS(=O)(=O)N(Cc1ncc(-c2nnc(C(F)F)o2)s1)c1cccnc1] |
| I-164 | 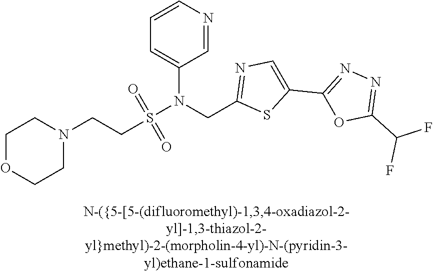 [SMILES: O=S(=O)(CCN1CCOCC1)N(Cc1ncc(-c2nnc(C(F)F)o2)s1)c1cccnc1] |
| I-165 | 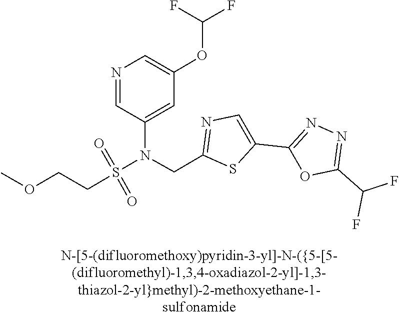 [SMILES: COCCS(=O)(=O)N(Cc1ncc(-c2nnc(C(F)F)o2)s1)c1cncc(OC(F)F)c1] |
| I-166 | 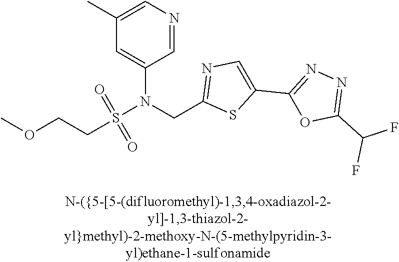 [SMILES: COCCS(=O)(=O)N(Cc1ncc(-c2nnc(C(F)F)o2)s1)c1cncc(C)c1] |
| I-167 | 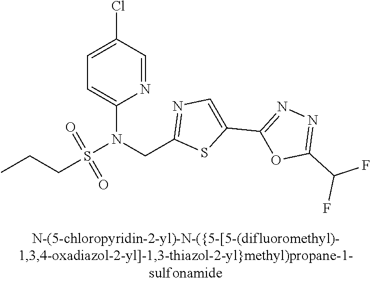 [SMILES: CCCS(=O)(=O)N(Cc1ncc(-c2nnc(C(F)F)o2)s1)c1ccc(Cl)cn1] |
| I-168 | 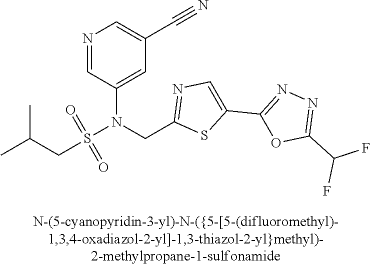 [SMILES: CC(C)CS(=O)(=O)N(Cc1ncc(-c2nnc(C(F)F)o2)s1)c1cncc(C#N)c1] |
| I-169 | 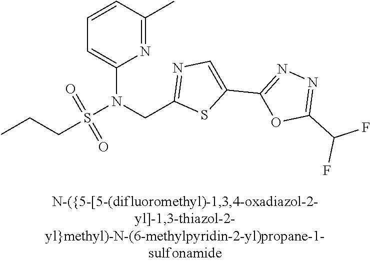 [SMILES: CCCS(=O)(=O)N(Cc1ncc(-c2nnc(C(F)F)o2)s1)c1cccc(C)n1] |
| I-170 | 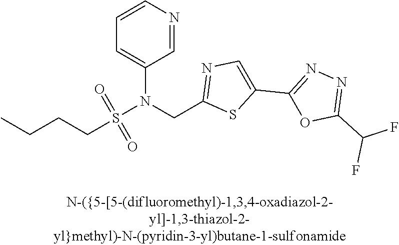 [SMILES: CCCCS(=O)(=O)N(Cc1ncc(-c2nnc(C(F)F)o2)s1)c1cccnc1] |
| I-171 | 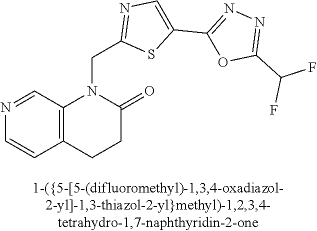 [SMILES: O=C1CCc2ccncc2N1Cc1ncc(-c2nnc(C(F)F)o2)s1] |
| I-172 | 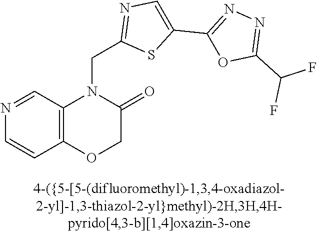 [SMILES: O=C1COc2ccncc2N1Cc1ncc(-c2nnc(C(F)F)o2)s1] |
| I-173 | 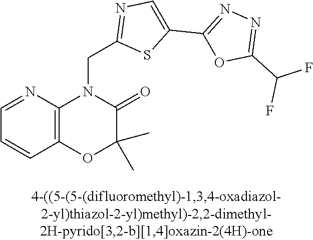 [SMILES: CC1(C)Oc2cccnc2N(Cc2ncc(-c3nnc(C(F)F)o3)s2)C1=O] |
| I-174 | 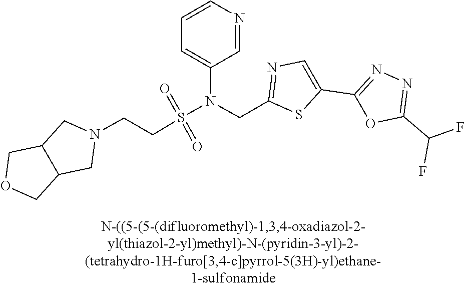 [SMILES: O=S(=O)(CCN1CC2COCC2C1)N(Cc1ncc(-c2nnc(C(F)F)o2)s1)c1cccnc1] |
| I-175 | 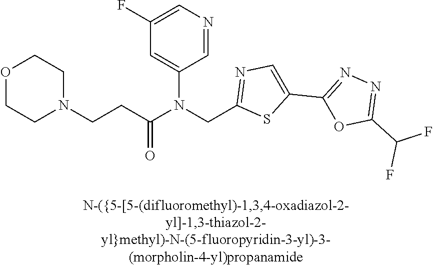 [SMILES: O=C(CCN1CCOCC1)N(Cc1ncc(-c2nnc(C(F)F)o2)s1)c1cncc(F)c1] |
| I-176 | 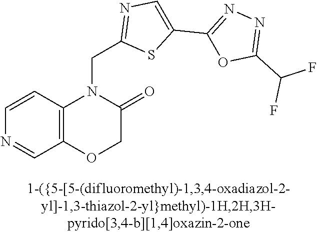 [SMILES: O=C1COc2cnccc2N1Cc1ncc(-c2nnc(C(F)F)o2)s1] |
| I-177 | 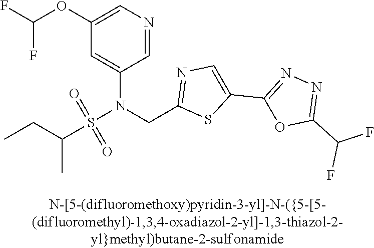 [SMILES: CCC(C)S(=O)(=O)N(Cc1ncc(-c2nnc(C(F)F)o2)s1)c1cncc(OC(F)F)c1] |
| I-178 | 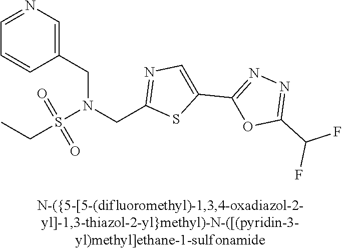 [SMILES: CCS(=O)(=O)N(Cc1cccnc1)Cc1ncc(-c2nnc(C(F)F)o2)s1] |
| I-179 | 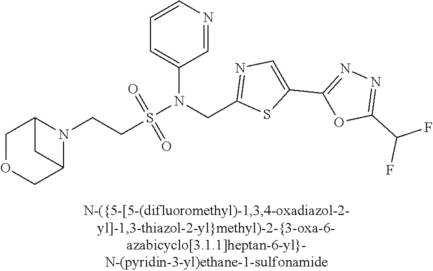 [SMILES: O=S(=O)(CCN1C2COCC1C2)N(Cc1ncc(-c2nnc(C(F)F)o2)s1)c1cccnc1] |
| I-180 | 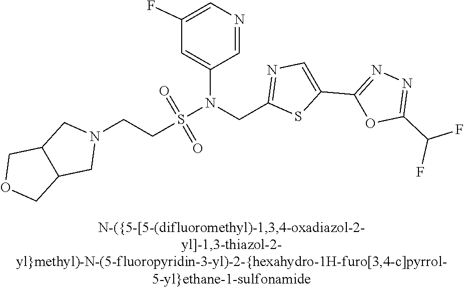 [SMILES: O=S(=O)(CCN1CC2COCC2C1)N(Cc1ncc(-c2nnc(C(F)F)o2)s1)c1cncc(F)c1] |
| I-181 | 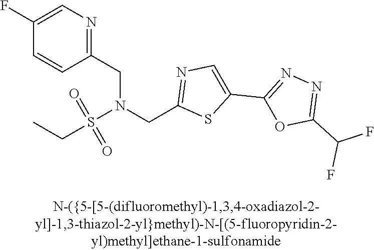 [SMILES: CCS(=O)(=O)N(Cc1ccc(F)cn1)Cc1ncc(-c2nnc(C(F)F)o2)s1] |
| I-182 | 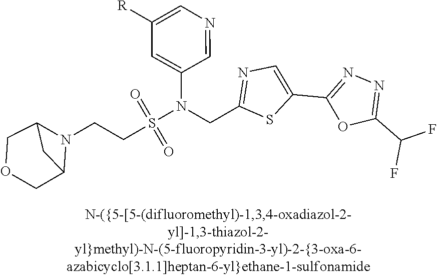 |
| I-183 | 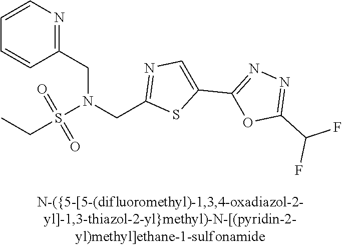 [SMILES: CCS(=O)(=O)N(Cc1ccccn1)Cc1ncc(-c2nnc(C(F)F)o2)s1] |
| I-184 | 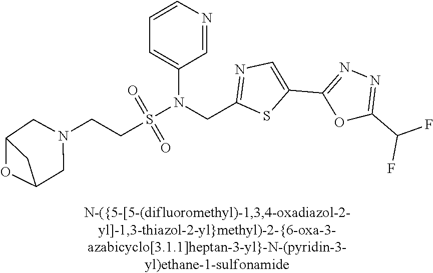 [SMILES: O=S(=O)(CCN1CC2CC(C1)O2)N(Cc1ncc(-c2nnc(C(F)F)o2)s1)c1cccnc1] |
| I-185 | 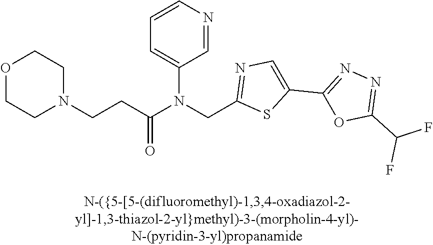 [SMILES: O=C(CCN1CCOCC1)N(Cc1ncc(-c2nnc(C(F)F)o2)s1)c1cccnc1] |
| I-186 | 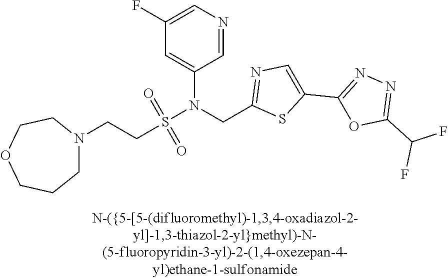 [SMILES: O=S(=O)(CCN1CCCOCC1)N(Cc1ncc(-c2nnc(C(F)F)o2)s1)c1cncc(F)c1] |
| I-187 | 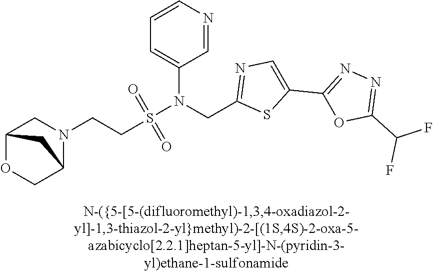 [SMILES: O=S(=O)(CCN1C[C@@H]2C[C@H]1CO2)N(Cc1ncc(-c2nnc(C(F)F)o2)s1)c1cccnc1] |
| I-188 | 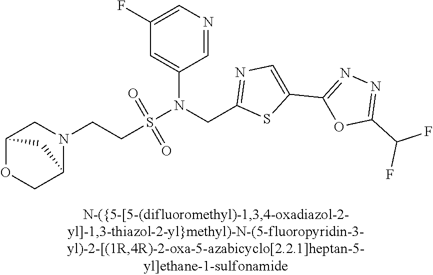 [SMILES: O=S(=O)(CCN1C[C@H]2C[C@@H]1CO2)N(Cc1ncc(-c2nnc(C(F)F)o2)s1)c1cncc(F)c1] |
| I-189 | 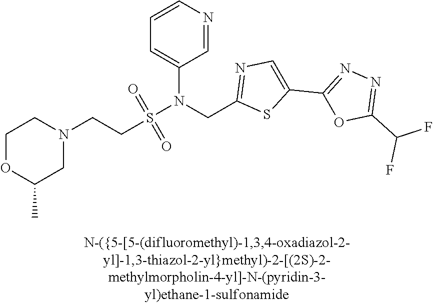 [SMILES: C[C@H]1CN(CCS(=O)(=O)N(Cc2ncc(-c3nnc(C(F)F)o3)s2)c2cccnc2)CCO1] |
| I-190 | 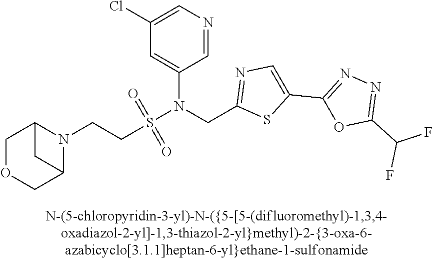 [SMILES: O=S(=O)(CCN1C2COCC1C2)N(Cc1ncc(-c2nnc(C(F)F)o2)s1)c1cncc(Cl)c1] |
| I-191 | 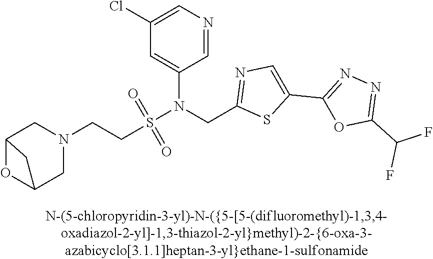 [SMILES: O=S(=O)(CCN1CC2CC(C1)O2)N(Cc1ncc(-c2nnc(C(F)F)o2)s1)c1cncc(Cl)c1] |
| I-192 | 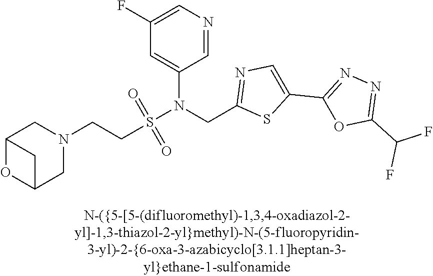 [SMILES: O=S(=O)(CCN1CC2CC(C1)O2)N(Cc1ncc(-c2nnc(C(F)F)o2)s1)c1cncc(F)c1] |
| I-193 | 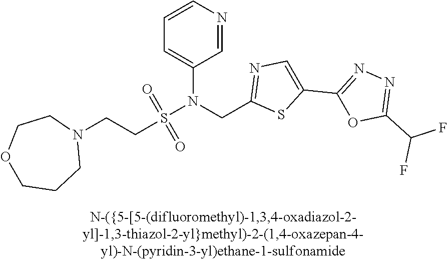 [SMILES: O=S(=O)(CCN1CCCOCC1)N(Cc1ncc(-c2nnc(C(F)F)o2)s1)c1cccnc1] |
| I-194 | 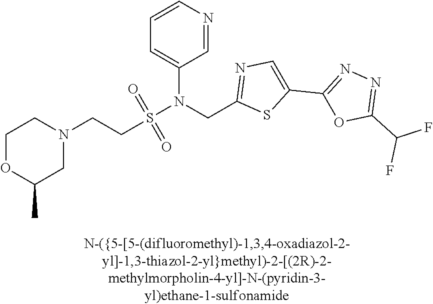 [SMILES: C[C@@H]1CN(CCS(=O)(=O)N(Cc2ncc(-c3nnc(C(F)F)o3)s2)c2cccnc2)CCO1] |
| I-195 | 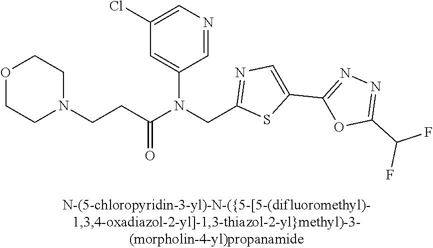 [SMILES: O=C(CCN1CCOCC1)N(Cc1ncc(-c2nnc(C(F)F)o2)s1)c1cncc(Cl)c1] |
| I-196 | 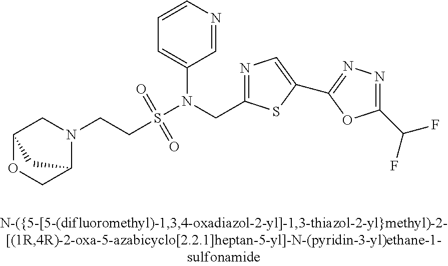 [SMILES: O=S(=O)(CCN1C[C@H]2C[C@@H]1CO2)N(Cc1ncc(-c2nnc(C(F)F)o2)s1)c1cccnc1] |
| I-197 | 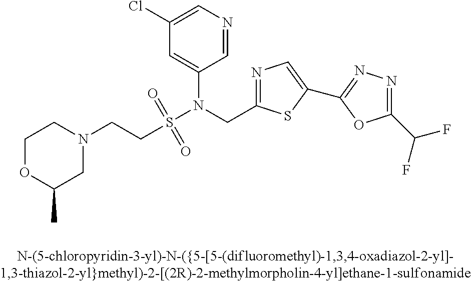 [SMILES: C[C@@H]1CN(CCS(=O)(=O)N(Cc2ncc(-c3nnc(C(F)F)o3)s2)c2cncc(Cl)c2)CCO1] |
| I-198 | 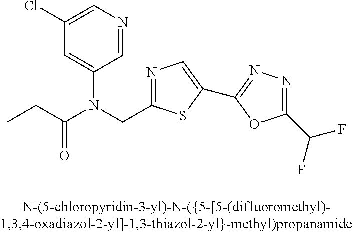 [SMILES: CCC(=O)N(Cc1ncc(-c2nnc(C(F)F)o2)s1)c1cncc(Cl)c1] |
| I-199 | 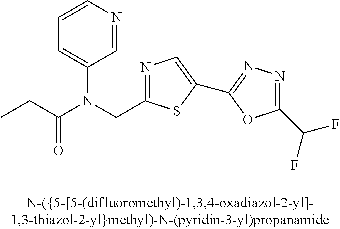 [SMILES: CCC(=O)N(Cc1ncc(-c2nnc(C(F)F)o2)s1)c1cccnc1] |
| I-200 | 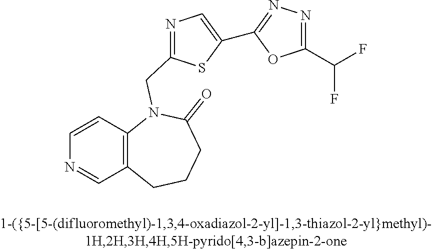 [SMILES: O=C1CCCc2cnccc2N1Cc1ncc(-c2nnc(C(F)F)o2)s1] |
| I-201 | 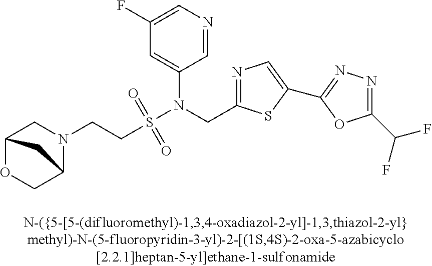 [SMILES: O=S(=O)(CCN1C[C@@H]2C[C@H]1CO2)N(Cc1ncc(-c2nnc(C(F)F)o2)s1)c1cncc(F)c1] |
| I-202 | 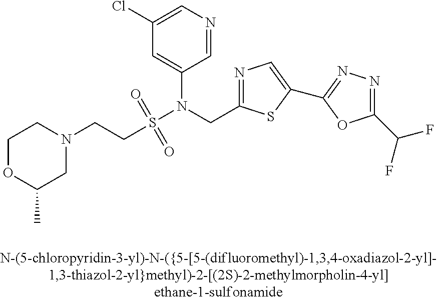 [SMILES: C[C@H]1CN(CCS(=O)(=O)N(Cc2ncc(-c3nnc(C(F)F)o3)s2)c2cncc(Cl)c2)CCO1] |
| I-203 | 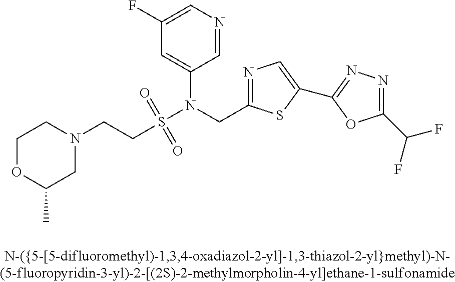 [SMILES: C[C@H]1CN(CCS(=O)(=O)N(Cc2ncc(-c3nnc(C(F)F)o3)s2)c2cncc(F)c2)CCO1] |
| I-204 | 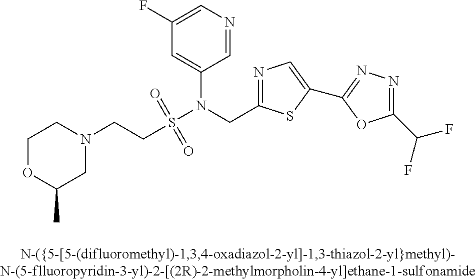 [SMILES: C[C@@H]1CN(CCS(=O)(=O)N(Cc2ncc(-c3nnc(C(F)F)o3)s2)c2cncc(F)c2)CCO1] |
| I-205 | 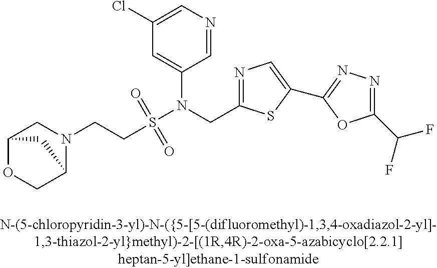 [SMILES: O=S(=O)(CCN1C[C@H]2C[C@@H]1CO2)N(Cc1ncc(-c2nnc(C(F)F)o2)s1)c1cncc(Cl)c1] |
| I-206 | 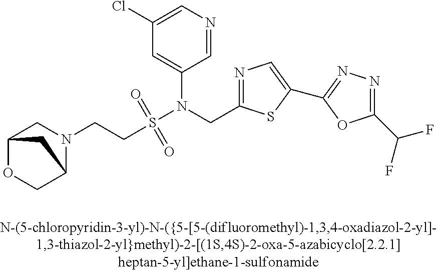 [SMILES: O=S(=O)(CCN1C[C@@H]2C[C@H]1CO2)N(Cc1ncc(-c2nnc(C(F)F)o2)s1)c1cncc(Cl)c1] |
| I-207 | 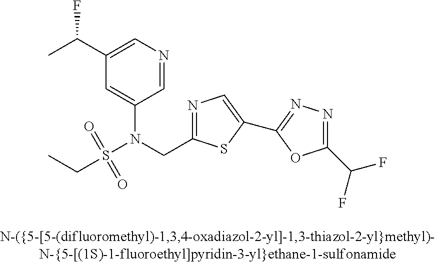 [SMILES: CCS(=O)(=O)N(Cc1ncc(-c2nnc(C(F)F)o2)s1)c1cncc([C@H](C)F)c1] |
| I-208 | 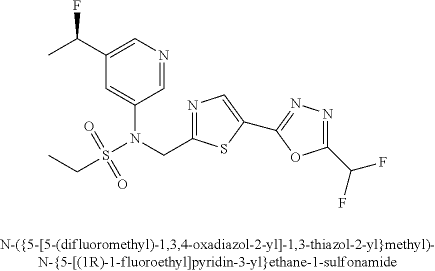 [SMILES: CCS(=O)(=O)N(Cc1ncc(-c2nnc(C(F)F)o2)s1)c1cncc([C@@H](C)F)c1] |
| I-209 | 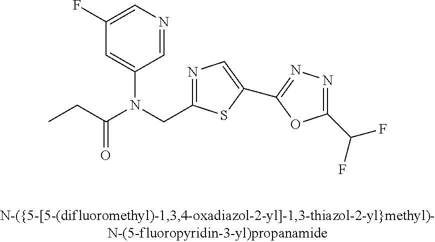 [SMILES: CCC(=O)N(Cc1ncc(-c2nnc(C(F)F)o2)s1)c1cncc(F)c1] |
| I-210 | 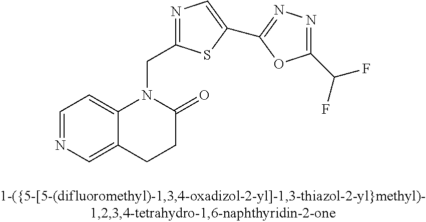 [SMILES: O=C1CCc2cnccc2N1Cc1ncc(-c2nnc(C(F)F)o2)s1] |
| I-211 | 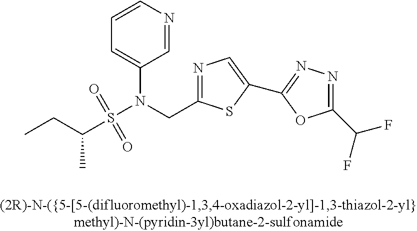 [SMILES: CC[C@@H](C)S(=O)(=O)N(Cc1ncc(-c2nnc(C(F)F)o2)s1)c1cccnc1] |
| I-212 | 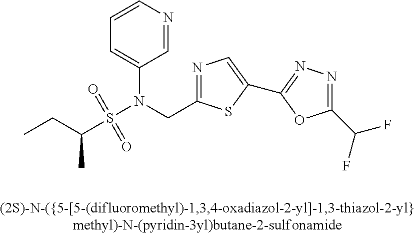 [SMILES: CC[C@H](C)S(=O)(=O)N(Cc1ncc(-c2nnc(C(F)F)o2)s1)c1cccnc1] |
| I-213 | 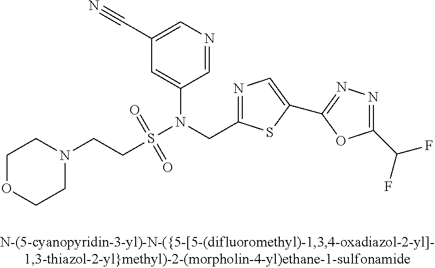 [SMILES: N#Cc1cncc(N(Cc2ncc(-c3nnc(C(F)F)o3)s2)S(=O)(=O)CCN2CCOCC2)c1] |
| I-214 | 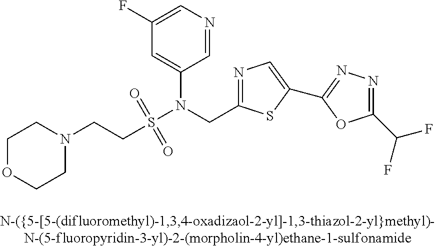 [SMILES: O=S(=O)(CCN1CCOCC1)N(Cc1ncc(-c2nnc(C(F)F)o2)s1)c1cncc(F)c1] |
| I-215 | 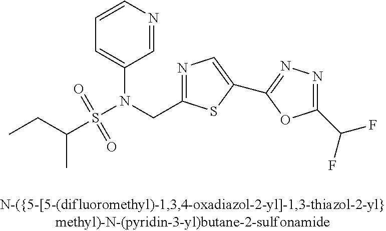 [SMILES: CCC(C)S(=O)(=O)N(Cc1ncc(-c2nnc(C(F)F)o2)s1)c1cccnc1] |
| I-216 | 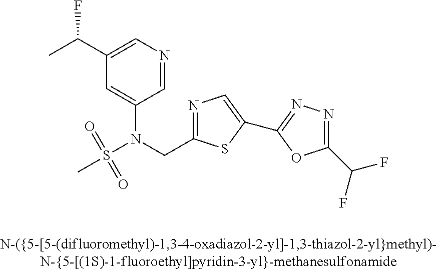 [SMILES: C[C@H](F)c1cncc(N(Cc2ncc(-c3nnc(C(F)F)o3)s2)S(C)(=O)=O)c1] |
| I-217 | 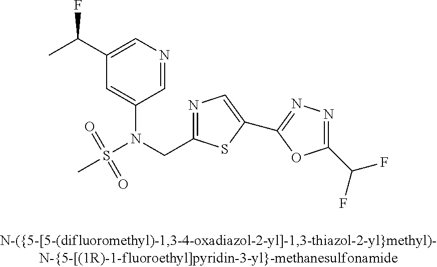 [SMILES: C[C@@H](F)c1cncc(N(Cc2ncc(-c3nnc(C(F)F)o3)s2)S(C)(=O)=O)c1] |
| I-218 | 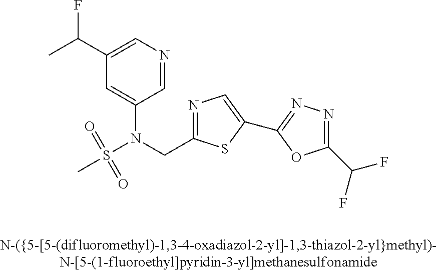 [SMILES: CC(F)c1cncc(N(Cc2ncc(-c3nnc(C(F)F)o3)s2)S(C)(=O)=O)c1] |
| I-219 | 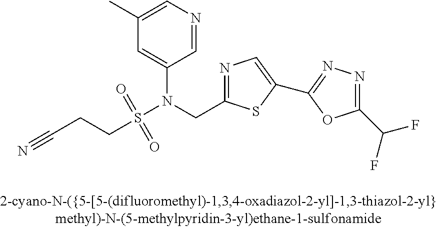 [SMILES: Cc1cncc(N(Cc2ncc(-c3nnc(C(F)F)o3)s2)S(=O)(=O)CCC#N)c1] |
| I-220 | 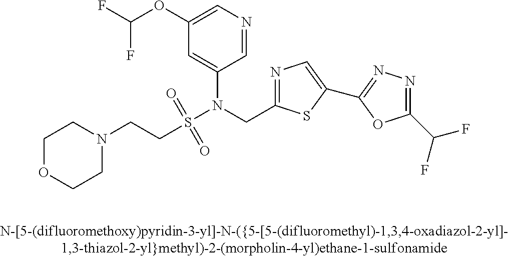 [SMILES: O=S(=O)(CCN1CCOCC1)N(Cc1ncc(-c2nnc(C(F)F)o2)s1)c1cncc(OC(F)F)c1] |
| I-221 | 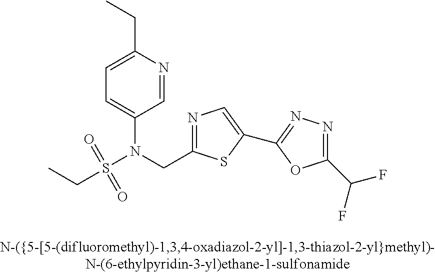 [SMILES: CCc1ccc(N(Cc2ncc(-c3nnc(C(F)F)o3)s2)S(=O)(=O)CC)cn1] |
| I-222 | 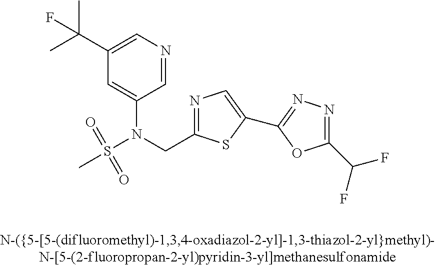 [SMILES: CC(C)(F)c1cncc(N(Cc2ncc(-c3nnc(C(F)F)o3)s2)S(C)(=O)=O)c1] |
| I-223 | 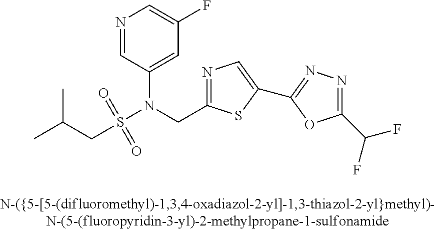 [SMILES: CC(C)CS(=O)(=O)N(Cc1ncc(-c2nnc(C(F)F)o2)s1)c1cncc(F)c1] |
| I-224 | 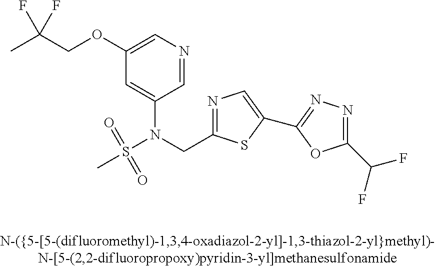 [SMILES: CC(F)(F)COc1cncc(N(Cc2ncc(-c3nnc(C(F)F)o3)s2)S(C)(=O)=O)c1] |
| I-225 | 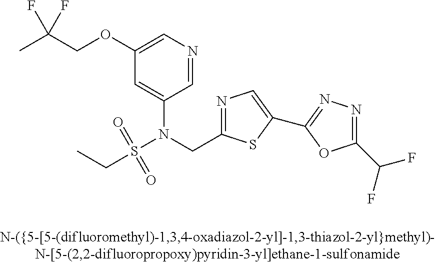 [SMILES: CCS(=O)(=O)N(Cc1ncc(-c2nnc(C(F)F)o2)s1)c1cncc(OCC(C)(F)F)c1] |
| I-226 | 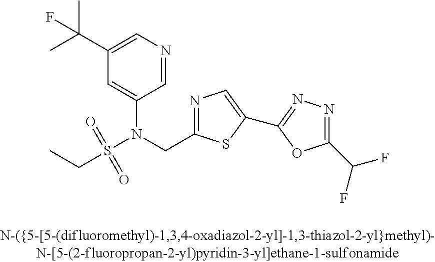 [SMILES: CCS(=O)(=O)N(Cc1ncc(-c2nnc(C(F)F)o2)s1)c1cncc(C(C)(C)F)c1] |
| I-227 | 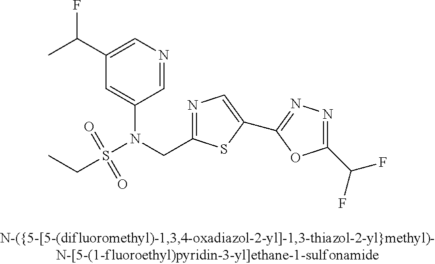 [SMILES: CCS(=O)(=O)N(Cc1ncc(-c2nnc(C(F)F)o2)s1)c1cncc(C(C)F)c1] |
| I-228 | 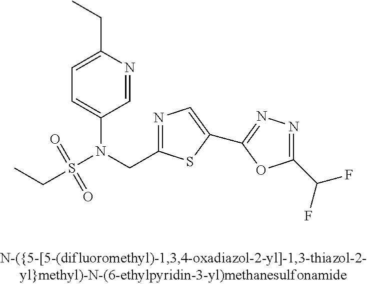 [SMILES: CCc1ccc(N(Cc2ncc(-c3nnc(C(F)F)o3)s2)S(=O)(=O)CC)cn1] |
| I-229 | 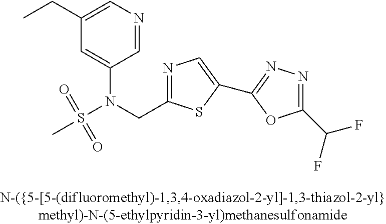 [SMILES: CCc1cncc(N(Cc2ncc(-c3nnc(C(F)F)o3)s2)S(C)(=O)=O)c1] |
| I-230 | 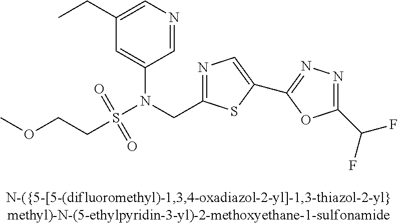 [SMILES: CCc1cncc(N(Cc2ncc(-c3nnc(C(F)F)o3)s2)S(=O)(=O)CCOC)c1] |
| I-231 | 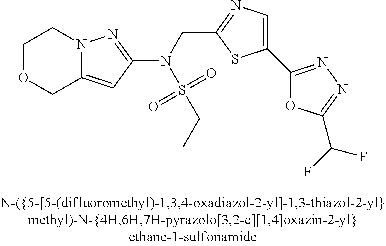 [SMILES: CCS(=O)(=O)N(Cc1ncc(-c2nnc(C(F)F)o2)s1)c1cc2n(n1)CCOC2] |
| I-232 | 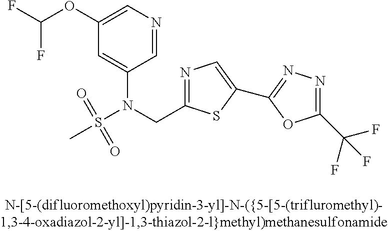 [SMILES: CS(=O)(=O)N(Cc1ncc(-c2nnc(C(F)(F)F)o2)s1)c1cncc(OC(F)F)c1] |
| I-233 | 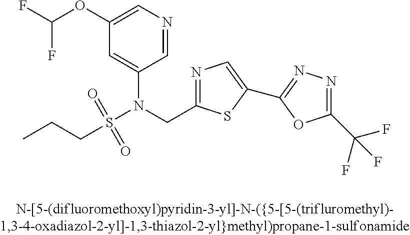 [SMILES: CCCS(=O)(=O)N(Cc1ncc(-c2nnc(C(F)(F)F)o2)s1)c1cncc(OC(F)F)c1] |
| I-234 | 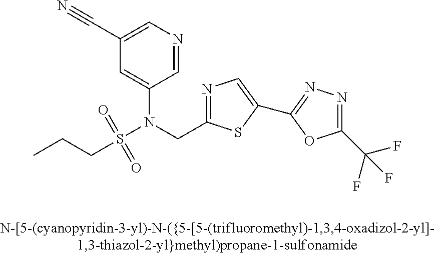 [SMILES: CCCS(=O)(=O)N(Cc1ncc(-c2nnc(C(F)(F)F)o2)s1)c1cncc(C#N)c1] |
| I-235 | 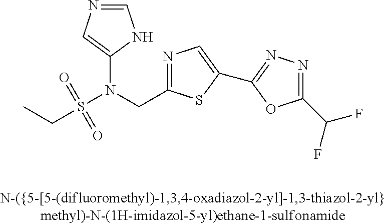 [SMILES: CCS(=O)(=O)N(Cc1ncc(-c2nnc(C(F)F)o2)s1)c1cnc[nH]1] |
| I-236 | 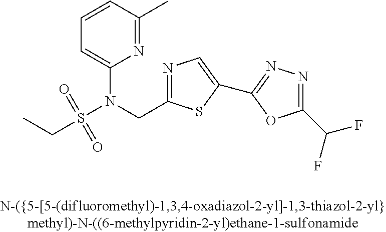 [SMILES: CCS(=O)(=O)N(Cc1ncc(-c2nnc(C(F)F)o2)s1)c1cccc(C)n1] |
| I-237 | 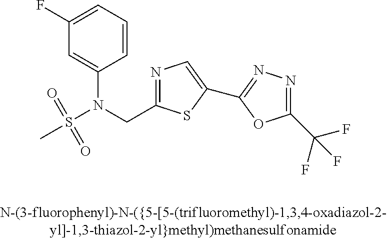 [SMILES: CS(=O)(=O)N(Cc1ncc(-c2nnc(C(F)(F)F)o2)s1)c1cccc(F)c1] |
| I-238 | 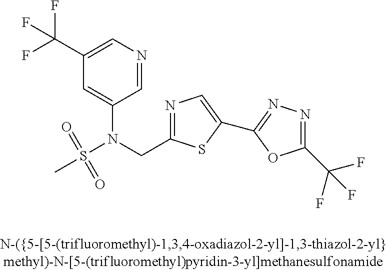 [SMILES: CS(=O)(=O)N(Cc1ncc(-c2nnc(C(F)(F)F)o2)s1)c1cncc(C(F)(F)F)c1] |
| I-239 | 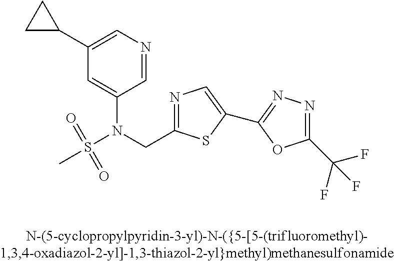 [SMILES: CS(=O)(=O)N(Cc1ncc(-c2nnc(C(F)(F)F)o2)s1)c1cncc(C2CC2)c1] |
| I-240 | 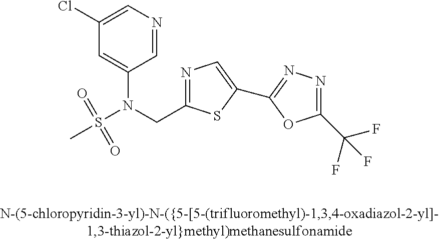 [SMILES: CS(=O)(=O)N(Cc1ncc(-c2nnc(C(F)(F)F)o2)s1)c1cncc(Cl)c1] |
| I-241 | 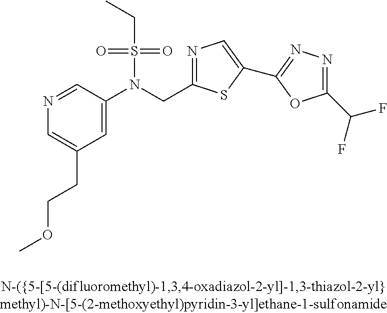 [SMILES: CCS(=O)(=O)N(Cc1ncc(-c2nnc(C(F)F)o2)s1)c1cncc(CCOC)c1] |
| I-242 | 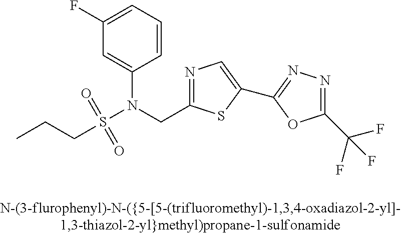 [SMILES: CCCS(=O)(=O)N(Cc1ncc(-c2nnc(C(F)(F)F)o2)s1)c1cccc(F)c1] |
| I-243 | 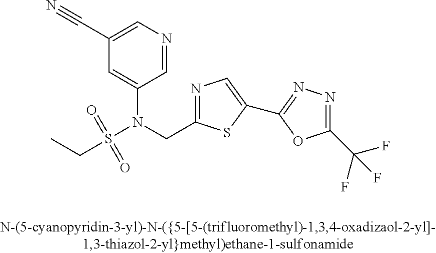 [SMILES: CCS(=O)(=O)N(Cc1ncc(-c2nnc(C(F)(F)F)o2)s1)c1cncc(C#N)c1] |
| I-244 | 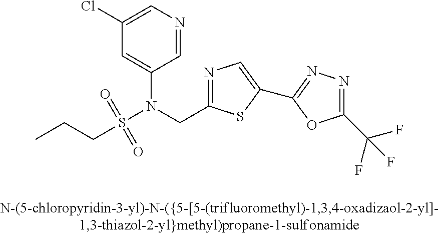 [SMILES: CCCS(=O)(=O)N(Cc1ncc(-c2nnc(C(F)(F)F)o2)s1)c1cncc(Cl)c1] |
| I-245 | 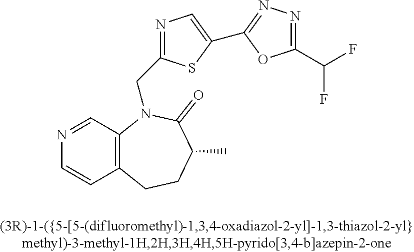 [SMILES: C[C@@H]1CCc2ccncc2N(Cc2ncc(-c3nnc(C(F)F)o3)s2)C1=O] |
| I-246 | 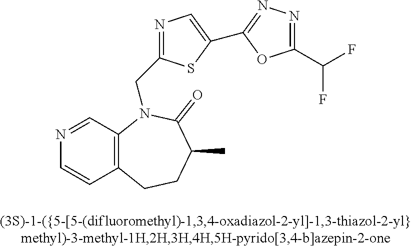 [SMILES: C[C@H]1CCc2ccncc2N(Cc2ncc(-c3nnc(C(F)F)o3)s2)C1=O] |
| I-247 | 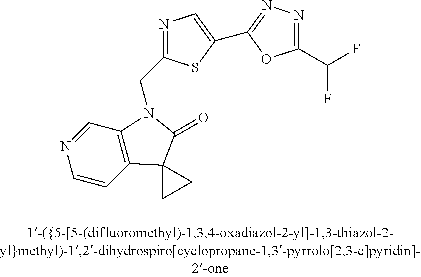 [SMILES: O=C1N(Cc2ncc(-c3nnc(C(F)F)o3)s2)c2cnccc2C12CC2] |
| I-248 | 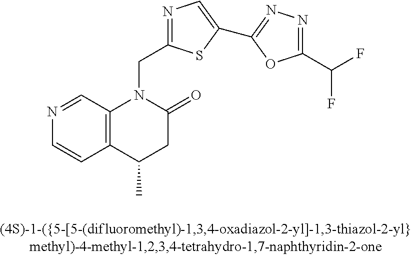 [SMILES: C[C@H]1CC(=O)N(Cc2ncc(-c3nnc(C(F)F)o3)s2)c2cnccc21] |
| I-249 | 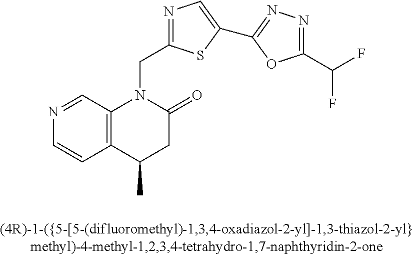 [SMILES: C[C@@H]1CC(=O)N(Cc2ncc(-c3nnc(C(F)F)o3)s2)c2cnccc21] |
| I-250 | 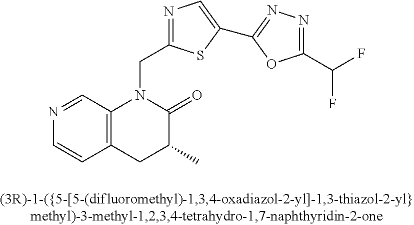 [SMILES: C[C@@H]1Cc2ccncc2N(Cc2ncc(-c3nnc(C(F)F)o3)s2)C1=O] |
| I-251 | 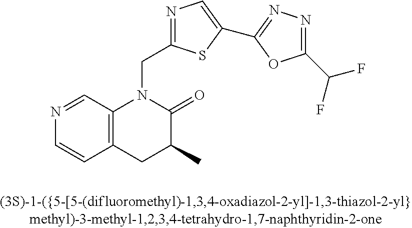 [SMILES: C[C@H]1Cc2ccncc2N(Cc2ncc(-c3nnc(C(F)F)o3)s2)C1=O] |
| I-252 | 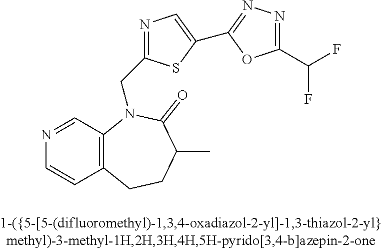 [SMILES: CC1CCc2ccncc2N(Cc2ncc(-c3nnc(C(F)F)o3)s2)C1=O] |
| I-253 | 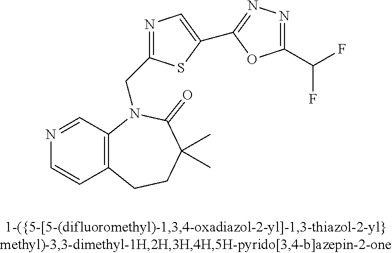 [SMILES: CC1(C)CCc2ccncc2N(Cc2ncc(-c3nnc(C(F)F)o3)s2)C1=O] |
| I-254 | 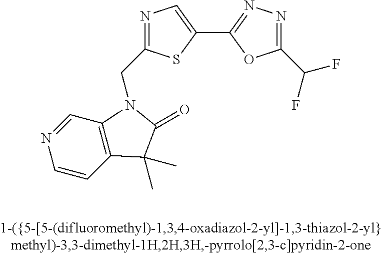 [SMILES: CC1(C)C(=O)N(Cc2ncc(-c3nnc(C(F)F)o3)s2)c2cnccc21] |
| 1-255 | 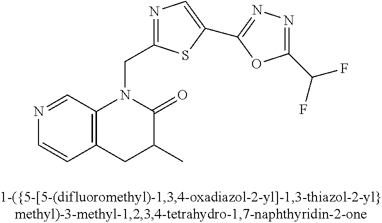 [SMILES: CC1Cc2ccncc2N(Cc2ncc(-c3nnc(C(F)F)o3)s2)C1=O] |
| I-256 | 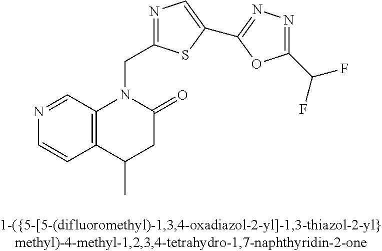 [SMILES: CC1CC(=O)N(Cc2ncc(-c3nnc(C(F)F)o3)s2)c2cnccc21] |
| I-257 | 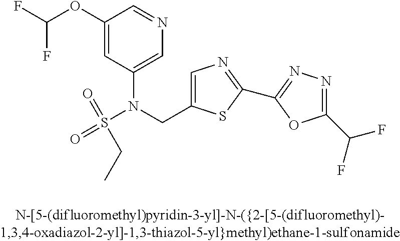 [SMILES: CCS(=O)(=O)N(Cc1cnc(-c2nnc(C(F)F)o2)s1)c1cncc(OC(F)F)c1] |
| I-258 | 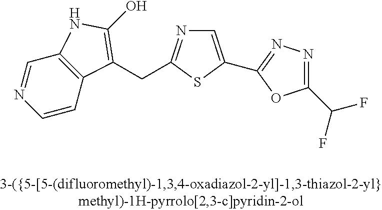 [SMILES: Oc1[nH]c2cnccc2c1Cc1ncc(-c2nnc(C(F)F)o2)s1] |
| I-259 | 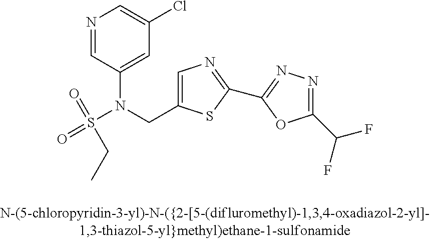 [SMILES: CCS(=O)(=O)N(Cc1cnc(-c2nnc(C(F)F)o2)s1)c1cncc(Cl)c1] |
| I-260 | 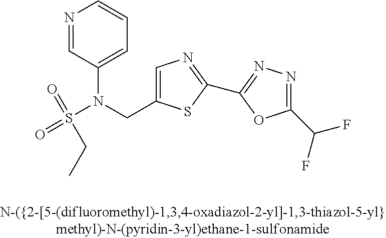 [SMILES: CCS(=O)(=O)N(Cc1cnc(-c2nnc(C(F)F)o2)s1)c1cccnc1] |
| I-261 | 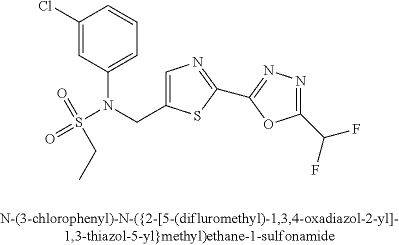 [SMILES: CCS(=O)(=O)N(Cc1cnc(-c2nnc(C(F)F)o2)s1)c1cccc(Cl)c1] |
| I-262 | 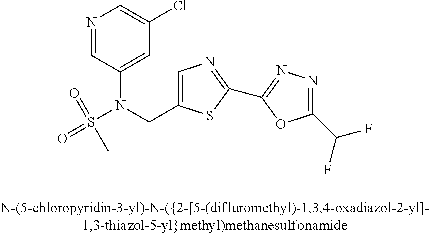 [SMILES: CS(=O)(=O)N(Cc1cnc(-c2nnc(C(F)F)o2)s1)c1cncc(Cl)c1] |
| I-263 | 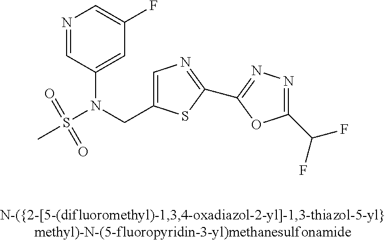 [SMILES: CS(=O)(=O)N(Cc1cnc(-c2nnc(C(F)F)o2)s1)c1cncc(F)c1] |
| I-264 | 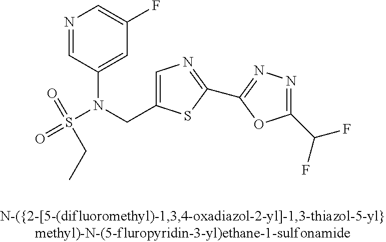 [SMILES: CCS(=O)(=O)N(Cc1cnc(-c2nnc(C(F)F)o2)s1)c1cncc(F)c1] |
| I-265 | 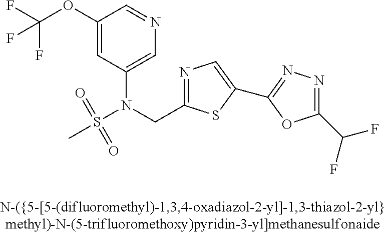 [SMILES: CS(=O)(=O)N(Cc1ncc(-c2nnc(C(F)F)o2)s1)c1cncc(OC(F)(F)F)c1] |
| I-266 | 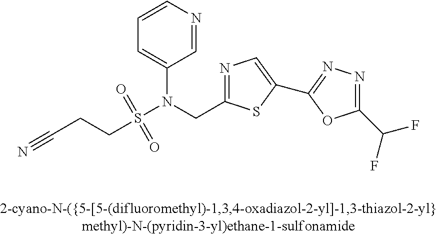 [SMILES: N#CCCS(=O)(=O)N(Cc1ncc(-c2nnc(C(F)F)o2)s1)c1cccnc1] |
| I-267 | 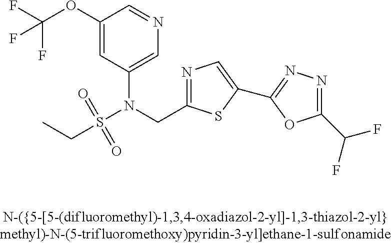 [SMILES: CCS(=O)(=O)N(Cc1ncc(-c2nnc(C(F)F)o2)s1)c1cncc(OC(F)(F)F)c1] |
| I-268 | 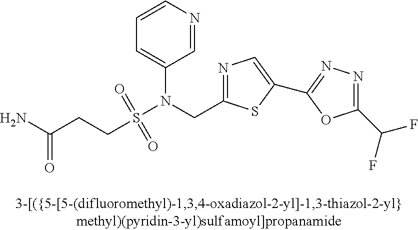 [SMILES: NC(=O)CCS(=O)(=O)N(Cc1ncc(-c2nnc(C(F)F)o2)s1)c1cccnc1] |
| I-269 | 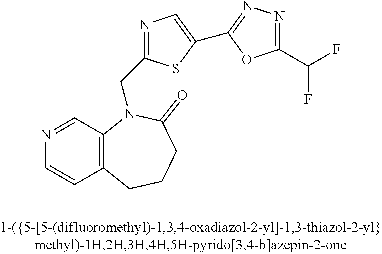 [SMILES: O=C1CCCc2ccncc2N1Cc1ncc(-c2nnc(C(F)F)o2)s1] |
| I-270 | 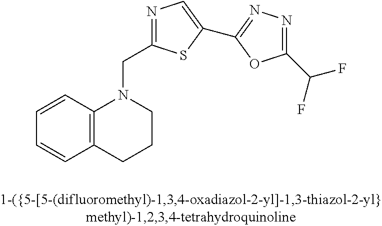 [SMILES: FC(F)c1nnc(-c2cnc(CN3CCCc4ccccc43)s2)o1] |

7. The method of embodiment 6, wherein the HDAC6 inhibitor is selected from the group consisting of:

| Cmpd | Structure/Name |
|---|---|
| I-2 | 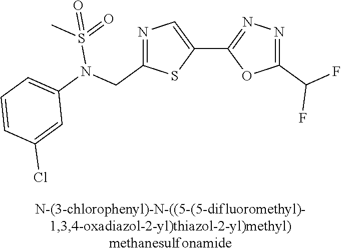 [SMILES: CS(=O)(=O)N(Cc1ncc(-c2nnc(C(F)F)o2)s1)c1cccc(Cl)c1] |
| I-3 | 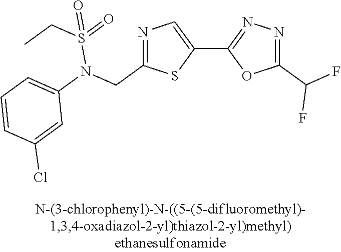 [SMILES: CCS(=O)(=O)N(Cc1ncc(-c2nnc(C(F)F)o2)s1)c1cccc(Cl)c1] |
| I-4 | 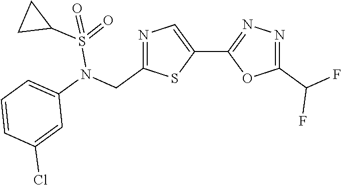 [SMILES: O=S(=O)(C1CC1)N(Cc1ncc(-c2nnc(C(F)F)o2)s1)c1cccc(Cl)c1] |
| | N-(3-chlorophenyl)-N-((5-(5-(difluoromethyl)-1,3,4-oxadiazol-2- |
| | yl)thiazol-2-yl)methyl)cyclopropanesulfonamide |
| I-5 | 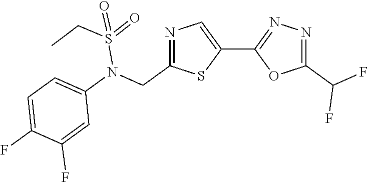 [SMILES: CCS(=O)(=O)N(Cc1ncc(-c2nnc(C(F)F)o2)s1)c1ccc(F)c(F)c1] |
| | N-((5-(5-(difluoromethyl)-1,3,4-oxadiazol-2-yl)thiazol-2-yl) |
| | methyl)-N-(3,4-difluorophenyl)ethanesulfonamide |
| I-6 | 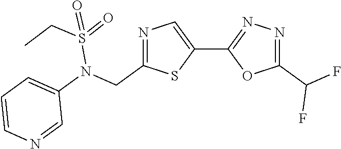 [SMILES: CCS(=O)(=O)N(Cc1ncc(-c2nnc(C(F)F)o2)s1)c1cccnc1] |
| | N-((5-(5-(difluoromethyl)-1,3,4-oxadiazol-2-yl)thiazol-2-yl) |
| | methyl)-N-(pyridin-3-yl)ethanesulfonamide |
| I-10 | 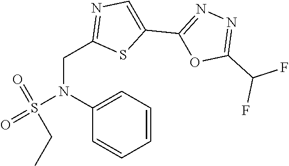 [SMILES: CCS(=O)(=O)N(Cc1ncc(-c2nnc(C(F)F)o2)s1)c1ccccc1] |
| | N-((5-(5-(difluoromethyl)-1,3,4-oxadiazol-2-yl)thiazol-2-yl) |
| | methyl)-N-phenylethanesulfonamide |
| I-13 | 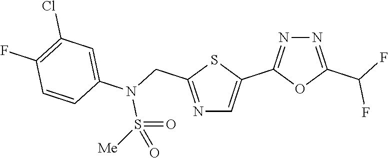 [SMILES: CS(=O)(=O)N(Cc1ncc(-c2nnc(C(F)F)o2)s1)c1ccc(F)c(Cl)c1] |
| | N-(3-chloro-4-fluorophenyl)-N-((5-(5-(difluoromethyl)- |
| | 1,3,4-oxadiazol-2-yl)thiazol-2-yl)methyl)methanesulfonamide |
| I-14 | 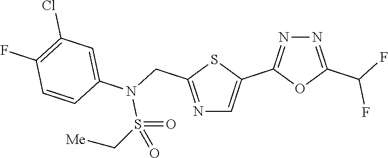 [SMILES: CCS(=O)(=O)N(Cc1ncc(-c2nnc(C(F)F)o2)s1)c1ccc(F)c(Cl)c1] |
| | N-(3-chloro-4-fluorophenyl)-N-((5-(5-(difluoromethyl)-1,3,4- |
| | oxadiazol-2-yl)thiazol-2-yl)methyl)ethanesulfonamide |
| I-15 | 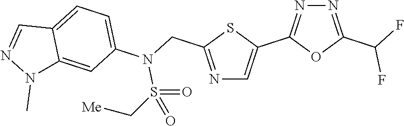 [SMILES: CCS(=O)(=O)N(Cc1ncc(-c2nnc(C(F)F)o2)s1)c1ccc2cnn(C)c2c1] |
| | N-((5-(5-(difluoromethyl)-1,3,4-oxadiazol-2-yl)thiazol-2-yl) |
| | methyl)-N-(1-methyl-1H-indazol-6-yl)ethanesulfonamide |
| I-16 | 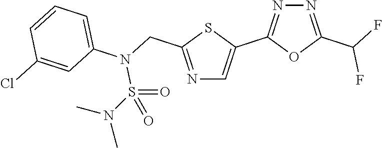 [SMILES: CN(C)S(=O)(=O)N(Cc1ncc(-c2nnc(C(F)F)o2)s1)c1cccc(Cl)c1] |
| | [(3-chlorophenyl)({5-[5-(difluoromethyl)-1,3,4-oxadiazol- |
| | 2-yl]-1,3-thiazol-2-yl}methyl)sulfamoyl]dimethylamine |
| I-30 | 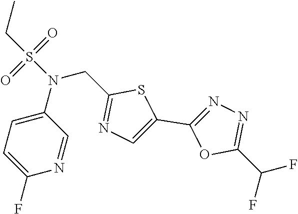 [SMILES: CCS(=O)(=O)N(Cc1ncc(-c2nnc(C(F)F)o2)s1)c1ccc(F)nc1] |
| | N-((5-(5-(difluoromethyl)-1,3,4-oxadiazol-2-yl)thiazol-2-yl) |
| | methyl)-N-(6-fluoropyridin-3-yl)ethanesulfonamide |
| I-34 | 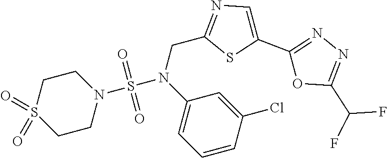 [SMILES: O=S1(=O)CCN(S(=O)(=O)N(Cc2ncc(-c3nnc(C(F)F)o3)s2)c2cccc(Cl)c2)CC1] |
| | N-(3-chlorophenyl)-N-((5-(5-(difluoromethyl)-1,3,4-oxadiazol-2- |
| | yl)thiazol-2-yl)methyl)thiomorpholine-4-sulfonamide 1,1-dioxide |
| I-35 | 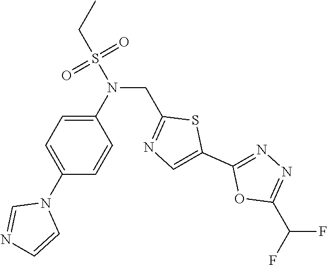 [SMILES: CCS(=O)(=O)N(Cc1ncc(-c2nnc(C(F)F)o2)s1)c1ccc(-n2ccnc2)cc1] |
| | N-(4-(1H-imidazol-1-yl)phenyl)-N-((5-(5-(difluoromethyl)- |
| | 1,3,4-oxadiazol-2-yl)thiazol-2-yl)methyl)ethanesulfonamide |
| I-36 | 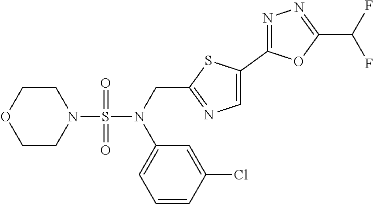 [SMILES: O=S(=O)(N1CCOCC1)N(Cc1ncc(-c2nnc(C(F)F)o2)s1)c1cccc(Cl)c1] |
| | N-(3-chlorophenyl)-N-((5-(5-(difluoromethyl)-1,3,4-oxadiazol-2- |
| | yl)thiazol-2-yl)methyl)morpholine-4-sulfonamide |
| I-37 | 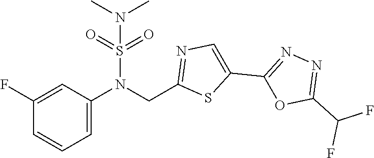 [SMILES: CN(C)S(=O)(=O)N(Cc1ncc(-c2nnc(C(F)F)o2)s1)c1cccc(F)c1] |
| I-38 | 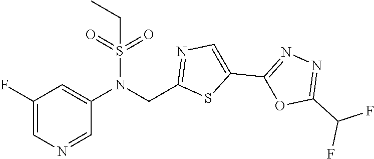 [SMILES: CCS(=O)(=O)N(Cc1ncc(-c2nnc(C(F)F)o2)s1)c1cncc(F)c1] |
| | N-((5-(5-(difluoromethyl)-1,3,4-oxadiazol-2-yl)thiazol- |
| | 2-yl)methyl)-N-(5-fluoropyridin-3-yl)ethanesulfonamide |
| I-39 | 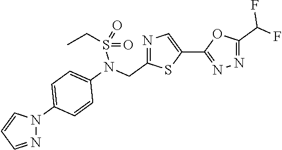 [SMILES: CCS(=O)(=O)N(Cc1ncc(-c2nnc(C(F)F)o2)s1)c1ccc(-n2cccn2)cc1] |
| | N-(4-(1H-pyrazol-1-yl)phenyl)-N-((5-(5-(difluoromethyl)-1,3, |
| | 4-oxadiazol-2-yl)thiazol-2-yl)methyl)ethanesulfonamide |
| I-52 | 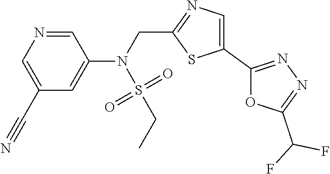 [SMILES: CCS(=O)(=O)N(Cc1ncc(-c2nnc(C(F)F)o2)s1)c1cncc(C#N)c1] |
| | N-(5-cyanopyridin-3-yl)-N-((5-(5-(difluoromethyl)-1,3,4- |
| | oxadiazol-2-yl)thiazol-2-yl)methyl)ethanesulfonamide |
| I-58 | 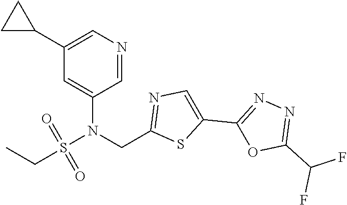 [SMILES: CCS(=O)(=O)N(Cc1ncc(-c2nnc(C(F)F)o2)s1)c1cncc(C2CC2)c1] |
| | N-(5-cyclopropylpyridin-3-yl)-N-((5-(5-(difluoromethyl)-1,3,4- |
| | oxadiazol-2-yl)thiazol-2-yl)methyl)ethanesulfonamide |
| I-59 | 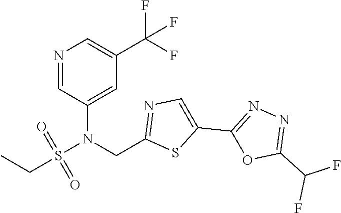 [SMILES: CCS(=O)(=O)N(Cc1ncc(-c2nnc(C(F)F)o2)s1)c1cncc(C(F)(F)F)c1] |
| | N-((5-(5-(difluoromethyl)-1,3,4-oxadiazol-2-yl)thiazol-2-yl) |
| | methyl)-N-(5-(trifluoromethyl)pyridin-3-yl)ethanesulfonamide |
| I-62 | 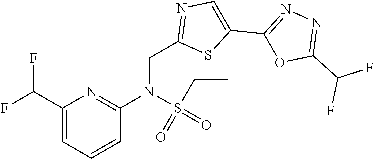 [SMILES: CCS(=O)(=O)N(Cc1ncc(-c2nnc(C(F)F)o2)s1)c1cccc(C(F)F)n1] |
| | N-((5-(5-(difluoromethyl)-1,3,4-oxadiazol-2-yl)thiazol-2-yl) |
| | methyl)-N-(6-(difluoromethyl)pyridin-2-yl)ethanesulfonamide |
| I-63 | 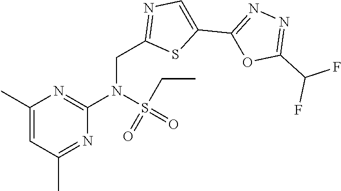 [SMILES: CCS(=O)(=O)N(Cc1ncc(-c2nnc(C(F)F)o2)s1)c1nc(C)cc(C)n1] |
| | N-((5-(5-(difluoromethyl)-1,3,4-oxadiazol-2-yl)thiazol-2-yl) |
| | methyl)-N-(4,6-dimethylpyrimidin-2-yl)ethanesulfonamide |
| I-65 | 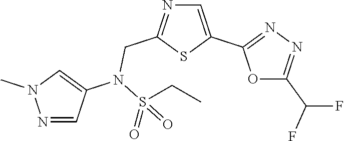 [SMILES: CCS(=O)(=O)N(Cc1ncc(-c2nnc(C(F)F)o2)s1)c1cnn(C)c1] |
| | N-((5-(5-(difluoromethyl)-1,3,4-oxadiazol-2-yl)thiazol-2-yl) |
| | methyl)-N-(1-methyl-1H-pyrazol-4-yl)ethanesulfonamide |
| I-66 | 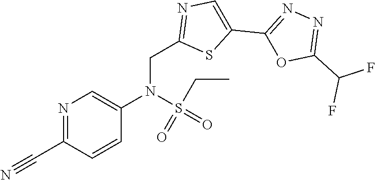 [SMILES: CCS(=O)(=O)N(Cc1ncc(-c2nnc(C(F)F)o2)s1)c1ccc(C#N)nc1] |
| | N-(6-cyanopyridin-3-yl)-N-((5-(5-(difluoromethyl)-1,3,4- |
| | oxadiazol-2-yl)thiazol-2-yl)methyl)ethanesulfonamide |
| I-70 | 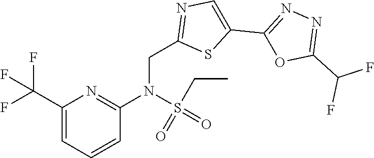 [SMILES: CCS(=O)(=O)N(Cc1ncc(-c2nnc(C(F)F)o2)s1)c1cccc(C(F)(F)F)n1] |
| | N-((5-(5-(difluoromethyl)-1,3,4-oxadiazol-2-yl)thiazol-2-yl) |
| | methyl)-N-(6-(trifluoromethyl)pyridin-2-yl)ethanesulfonamide |
| I-74 | 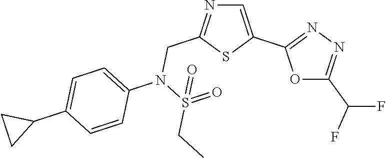 [SMILES: CCS(=O)(=O)N(Cc1ncc(-c2nnc(C(F)F)o2)s1)c1ccc(C2CC2)cc1] |
| | N-(6-cyclopropylpyridin-3-yl)-N-((5-(5-(difluoromethyl)-1,3,4- |
| | oxadiazol-2-yl)thiazol-2-yl)methyl)ethanesulfonamide |
| I-75 | 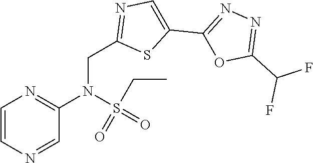 [SMILES: CCS(=O)(=O)N(Cc1ncc(-c2nnc(C(F)F)o2)s1)c1cnccn1] |
| | N-((5-(5-(difluoromethyl)-1,3,4-oxadiazol-2-yl)thiazol-2-yl) |
| | methyl)-N-(pyrazin-2-yl)ethanesulfonamide |
| I-80 | 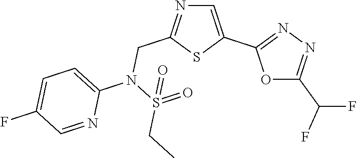 [SMILES: CCS(=O)(=O)N(Cc1ncc(-c2nnc(C(F)F)o2)s1)c1ccc(F)cn1] |
| | N-((5-(5-(difluoromethyl)-1,3,4-oxadiazol-2-yl)thiazol-2-yl) |
| | methyl)-N-(5-fluoropyridin-2-yl)ethanesulfonamide |
| I-91 | 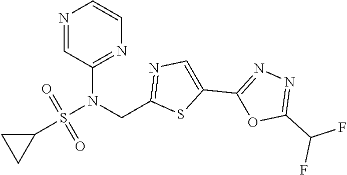 [SMILES: O=S(=O)(C1CC1)N(Cc1ncc(-c2nnc(C(F)F)o2)s1)c1cnccn1] |
| | N-((5-(5-(difluoromethyl)-1,3,4-oxadiazol-2-yl)thiazol-2- |
| | yl)methyl)-N-(pyrazin-2-yl)cyclopropanesulfonamide |
| I-92 | 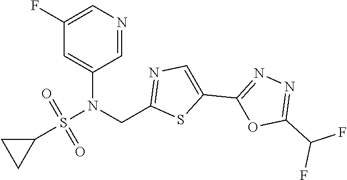 [SMILES: O=S(=O)(C1CC1)N(Cc1ncc(-c2nnc(C(F)F)o2)s1)c1cncc(F)c1] |
| | N-((5-(5-(difluoromethyl)-1,3,4-oxadiazol-2-yl)thiazol-2-yl) |
| | methyl)-N-(5-fluoropyridin-3-yl)cyclopropanesulfonamide |
| I-94 | 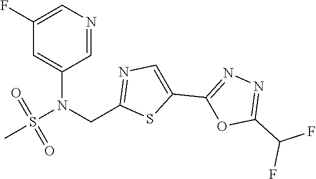 [SMILES: CS(=O)(=O)N(Cc1ncc(-c2nnc(C(F)F)o2)s1)c1cncc(F)c1] |
| | N-((5-(5-(difluoromethyl)-1,3,4-oxadiazol-2-yl)thiazol-2-yl) |
| | methyl)-N-(5-fluoropyridin-3-yl)methanesulfonamide |
| I-95 | 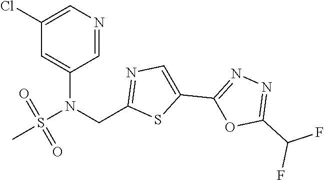 [SMILES: CS(=O)(=O)N(Cc1ncc(-c2nnc(C(F)F)o2)s1)c1cncc(Cl)c1] |
| | N-(5-chloropyridin-3-yl)-N-((5-(5-(difluoromethyl)-1,3,4- |
| | oxadiazol-2-yl)thiazol-2-yl)methyl)methanesulfonamide |
| I-96 | 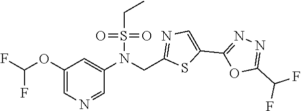 [SMILES: CCS(=O)(=O)N(Cc1ncc(-c2nnc(C(F)F)o2)s1)c1cncc(OC(F)F)c1] |
| | N-(5-(difluoromethoxy)pyridin-3-yl)-N-((5-(5-(difluoromethyl)- |
| | 1,3,4-oxadiazol-2-yl)thiazol-2-yl)methyl)ethanesulfonamide |
| I-97 | 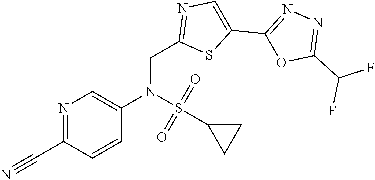 [SMILES: N#Cc1ccc(N(Cc2ncc(-c3nnc(C(F)F)o3)s2)S(=O)(=O)C2CC2)cn1] |
| | N-(6-cyanopyridin-3-yl)-N-((5-(5-(difluoromethyl)-1,3,4- |
| | oxadiazol-2-yl)thiazol-2-yl)methyl)cyclopropanesulfonamide |
| I-99 | 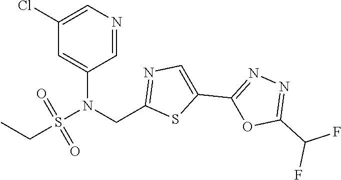 [SMILES: CCS(=O)(=O)N(Cc1ncc(-c2nnc(C(F)F)o2)s1)c1cncc(Cl)c1] |
| | N-(5-chloropyridin-3-yl)-N-((5-(5-(difluoromethyl)-1,3,4- |
| | oxadiazol-2-yl)thiazol-2-yl)methyl)ethanesulfonamide |
| I-103 | 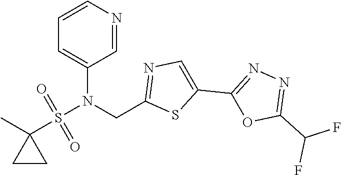 [SMILES: CC1(S(=O)(=O)N(Cc2ncc(-c3nnc(C(F)F)o3)s2)c2cccnc2)CC1] |
| | N-((5-(5-(difluoromethyl)-1,3,4-oxadiazol-2-yl)thiazol-2-yl) |
| | methyl)-1-methyl-N-(pyridin-3-yl)cyclopropane-1-sulfonamide |
| I-105 | 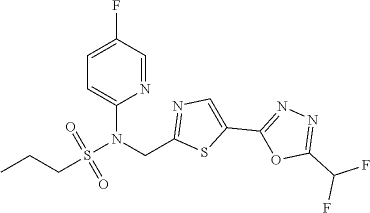 [SMILES: CCCS(=O)(=O)N(Cc1ncc(-c2nnc(C(F)F)o2)s1)c1ccc(F)cn1] |
| | N-({5-[5-(difluoromethyl)-1,3,4-oxadiazol-2-yl]-1,3-thiazol-2- |
| | yl}methyl)-N-(5-fluoropyridin-2-yl)propane-1-sulfonamide |
| I-107 | 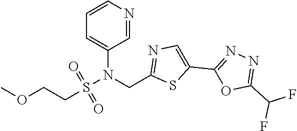 [SMILES: COCCS(=O)(=O)N(Cc1ncc(-c2nnc(C(F)F)o2)s1)c1cccnc1] |
| | N-({5-[5-(difluoromethyl)-1,3,4-oxadiazol-2-yl]-1,3-thiazol-2- |
| | yl}methyl)-2-methoxy-N-(pyridin-3-yl)ethane-1-sulfonamide |
| I-108 | 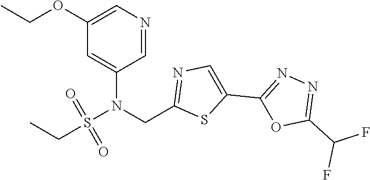 [SMILES: CCOc1cncc(N(Cc2ncc(-c3nnc(C(F)F)o3)s2)S(=O)(=O)CC)c1] |
| | N-({5-[5-(difluoromethyl)-1,3,4-oxadiazol-2-yl]-1,3-thiazol-2- |
| | yl}methyl)-N-(5-ethoxypyridin-3-yl)ethane-1-sulfonamide |
| I-109 | 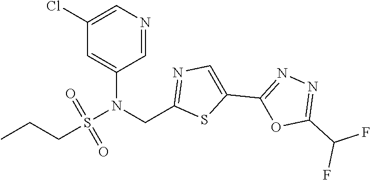 [SMILES: CCCS(=O)(=O)N(Cc1ncc(-c2nnc(C(F)F)o2)s1)c1cncc(Cl)c1] |
| | N-(5-chloropyridin-3-yl)-N-({5-[5-(difluoromethyl)-1,3,4- |
| | oxadiazol-2-yl]-1,3-thiazol-2-yl}methyl)propane-1-sulfonamide |
| I-110 | 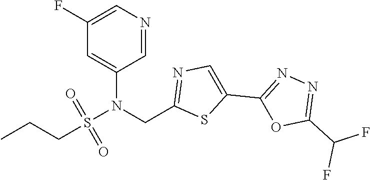 [SMILES: CCCS(=O)(=O)N(Cc1ncc(-c2nnc(C(F)F)o2)s1)c1cncc(F)c1] |
| | N-({5-[5-(difluoromethyl)-1,3,4-oxadiazol-2-yl]-1,3-thiazol-2- |
| | yl}methyl)-N-(5-fluoropyridin-3-yl)propane-1-sulfonamide |
| I-112 | 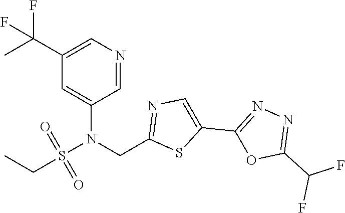 [SMILES: CCS(=O)(=O)N(Cc1ncc(-c2nnc(C(F)F)o2)s1)c1cncc(C(C)(F)F)c1] |
| | N-[5-(1,1-difluoroethyl)pyridin-3-yl]-N-({5-[5- |
| | (difluoromethyl)-1,3,4-oxadiazol-2-yl]-1,3-thiazol-2-yl} |
| | methyl)ethane-1-sulfonamide |
| I-115 | 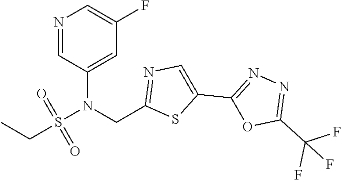 [SMILES: CCS(=O)(=O)N(Cc1ncc(-c2nnc(C(F)(F)F)o2)s1)c1cncc(F)c1] |
| | N-(5-fluoropyridin-3-yl)-N-({5-[5-(trifluoromethyl)-1,3,4- |
| | oxadiazol-2-yl]-1,3-thiazol-2-yl}methyl)ethane-1-sulfonamide |
| I-117 | 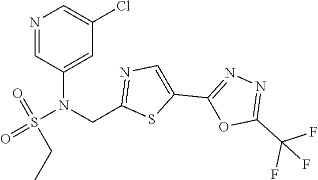 [SMILES: CCS(=O)(=O)N(Cc1ncc(-c2nnc(C(F)(F)F)o2)s1)c1cncc(Cl)c1] |
| | N-(5-chloropyridin-3-yl)-N-({5-[5-(trifluoromethyl)-1,3,4- |
| | oxadiazol-2-yl]-1,3-thiazol-2-yl}methyl)ethane-1-sulfonamide |
| I-118 | 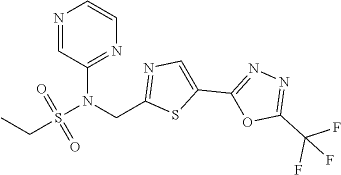 [SMILES: CCS(=O)(=O)N(Cc1ncc(-c2nnc(C(F)(F)F)o2)s1)c1cnccn1] |
| | N-(pyrazin-2-yl)-N-({5-[5-(trifluoromethyl)-1,3,4-oxadiazol- |
| | 2-yl]-1,3-thiazol-2-yl}methyl)ethane-1-sulfonamide |
| I-122 | 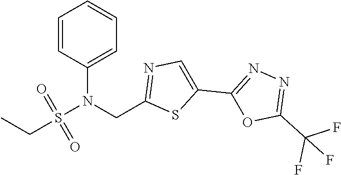 [SMILES: CCS(=O)(=O)N(Cc1ncc(-c2nnc(C(F)(F)F)o2)s1)c1ccccc1] |
| | N-phenyl-N-({5-[5-(trifluoromethyl)-1,3,4-oxadiazol-2-yl]- |
| | 1,3-thiazol-2-yl}methyl)ethane-1-sulfonamide |
| I-127 | 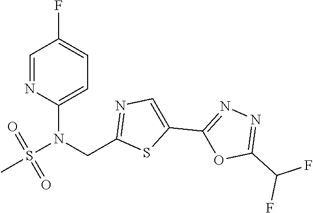 [SMILES: CS(=O)(=O)N(Cc1ncc(-c2nnc(C(F)F)o2)s1)c1ccc(F)cn1] |
| | N-({5-[5-(difluoromethyl)-1,3,4-oxadiazol-2-yl]-1,3-thiazol-2- |
| | yl}methyl)-N-(5-fluoropyridin-2-yl)methanesulfonamide |
| I-131 | 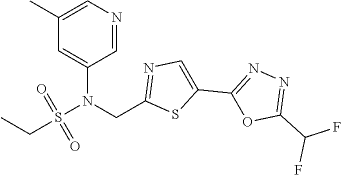 [SMILES: CCS(=O)(=O)N(Cc1ncc(-c2nnc(C(F)F)o2)s1)c1cncc(C)c1] |
| | N-({5-[5-(difluoromethyl)-1,3,4-oxadiazol-2-yl]-1,3-thiazol-2- |
| | yl}methyl)-N-(5-methylpyridin-3-yl)ethane-1-sulfonamide |
| I-132 | 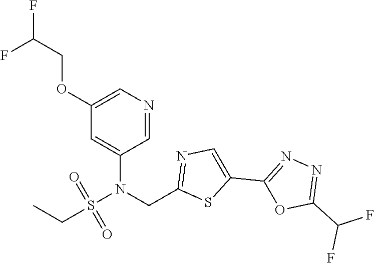 [SMILES: CCS(=O)(=O)N(Cc1ncc(-c2nnc(C(F)F)o2)s1)c1cncc(OCC(F)F)c1] |
| | N-[5-(2,2-difluoroethoxy)pyridin-3-yl]-N-({5-[5- |
| | (difluoromethyl)-1,3,4-oxadiazol-2-yl]-1,3-thiazol-2-yl} |
| | methyl)ethane-1-sulfonamide |
| I-136 | 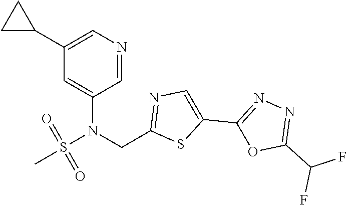 [SMILES: CS(=O)(=O)N(Cc1ncc(-c2nnc(C(F)F)o2)s1)c1cncc(C2CC2)c1] |
| | N-(5-cyclopropylpyridin-3-yl)-N-({5-[5-(difluoromethyl)-1,3,4- |
| | oxadiazol-2-yl]-1,3-thiazol-2-yl}methyl)methanesulfonamide |
| I-137 | 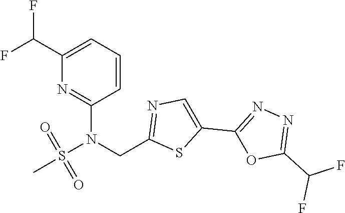 [SMILES: CS(=O)(=O)N(Cc1ncc(-c2nnc(C(F)F)o2)s1)c1cccc(C(F)F)n1] |
| | N-({5-[5-(difluoromethyl)-1,3,4-oxadiazol-2-yl]-1,3-thiazol-2-yl} |
| | methyl)-N-[6-(difluoromethyl)pyridin-2-yl]methanesulfonamide |
| I-142 | 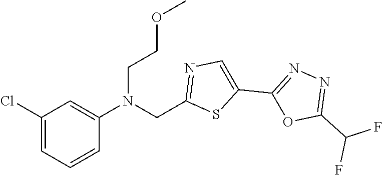 [SMILES: COCCN(Cc1ncc(-c2nnc(C(F)F)o2)s1)c1cccc(Cl)c1] |
| | 3-chloro-N-({5-[5-(difluoromethyl)-1,3,4-oxadiazol-2-yl]-1,3- |
| | thiazol-2-yl}methyl)-N-(2-methoxyethyl)aniline |
| I-143 | 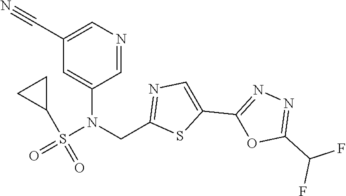 [SMILES: N#Cc1cncc(N(Cc2ncc(-c3nnc(C(F)F)o3)s2)S(=O)(=O)C2CC2)c1] |
| | N-(5-cyanopyridin-3-yl)-N-({5-[5-(difluoromethyl)-1,3,4- |
| | oxadiazol-2-yl]-1,3-thiazol-2-yl}methyl) |
| | cyclopropanesulfonamide |
| I-145 | 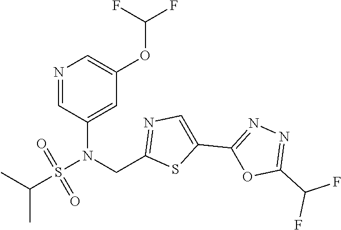 [SMILES: CC(C)S(=O)(=O)N(Cc1ncc(-c2nnc(C(F)F)o2)s1)c1cncc(OC(F)F)c1] |
| | N-[5-(difluoromethoxy)pyridin-3-yl]-N-({5- |
| | [5-(difluoromethyl)-1,3,4-oxadiazol-2-yl]- |
| | 1,3-thiazol-2-yl}methyl)propane-2-sulfonamide |
| I-146 | 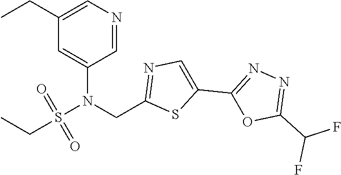 [SMILES: CCc1cncc(N(Cc2ncc(-c3nnc(C(F)F)o3)s2)S(=O)(=O)CC)c1] |
| | N-({5-[5-(difluoromethyl)-1,3,4-oxadiazol-2-yl]-1,3-thiazol-2- |
| | yl}methyl)-N-(5-ethylpyridin-3-yl)ethane-1-sulfonamide |
| I-147 | 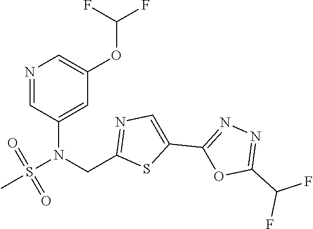 [SMILES: CS(=O)(=O)N(Cc1ncc(-c2nnc(C(F)F)o2)s1)c1cncc(OC(F)F)c1] |
| | N-[5-(difluoromethoxy)pyridin-3-yl]-N-({5-[5- |
| | (difluoromethyl)-1,3,4-oxadiazol-2-yl]-1,3- |
| | thiazol-2-yl}methyl)methanesulfonamide |
| I-148 | 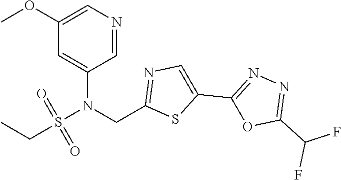 [SMILES: CCS(=O)(=O)N(Cc1ncc(-c2nnc(C(F)F)o2)s1)c1cncc(OC)c1] |
| | N-({5-[5-(difluoromethyl)-1,3,4-oxadiazol-2-yl]-1,3-thiazol-2- |
| | yl}methyl)-N-(5-methoxypyridin-3-yl)ethane-1-sulfonamide |
| I-149 | 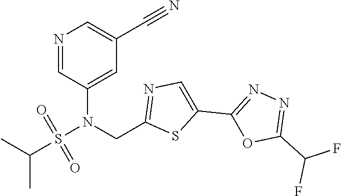 [SMILES: CC(C)S(=O)(=O)N(Cc1ncc(-c2nnc(C(F)F)o2)s1)c1cncc(C#N)c1] |
| | N-(5-cyanopyridin-3-yl)-N-({5-[5-(difluoromethyl)-1,3,4-oxadiazol- |
| | 2-yl]-1,3-thiazol-2-yl}methyl)propane-2-sulfonamide |
| I-150 | 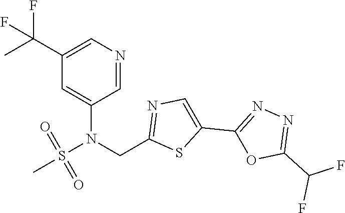 [SMILES: CC(F)(F)c1cncc(N(Cc2ncc(-c3nnc(C(F)F)o3)s2)S(C)(=O)=O)c1] |
| | N-[5-(1,1-difluoroethyl)pyridin-3-yl]-N-({5-[5- |
| | (difluoromethyl)-1,3,4-oxadiazol-2-yl]-1,3- |
| | thiazol-2-yl}methyl)methanesulfonamide |
| I-156 | 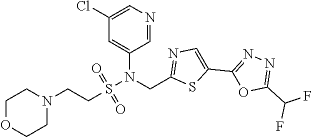 [SMILES: O=S(=O)(CCN1CCOCC1)N(Cc1ncc(-c2nnc(C(F)F)o2)s1)c1cncc(Cl)c1] |
| | N-(5-chloropyridin-3-yl)-N-({5-[5-(difluoromethyl)-1,3,4- |
| | oxadiazol-2-yl]-1,3-thiazol-2-yl}methyl)- |
| | 2-(morpholin-4-yl)ethane-1-sulfonamide |
| I-157 | 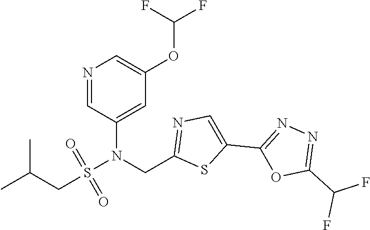 [SMILES: CC(C)CS(=O)(=O)N(Cc1ncc(-c2nnc(C(F)F)o2)s1)c1cncc(OC(F)F)c1] |
| | N-[5-(difluoromethoxy)pyridin-3-yl]-N-({5-[5- |
| | (difluoromethyl)-1,3,4-oxadiazol-2-yl]-1,3-thiazol-2-yl} |
| | methyl)-2-methylpropane-1-sulfonamide |
| I-158 | 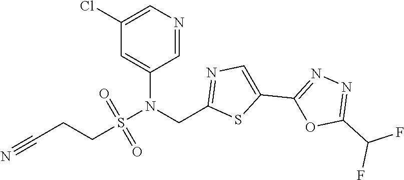 [SMILES: N#CCCS(=O)(=O)N(Cc1ncc(-c2nnc(C(F)F)o2)s1)c1cncc(Cl)c1] |
| | N-(5-chloropyridin-3-yl)-2-cyano-N-({5-[5- |
| | (difluoromethyl)-1,3,4-oxadiazol-2-yl]-1,3- |
| | thiazol-2-yl}methyl)ethane-1-sulfonamide |
| I-159 | 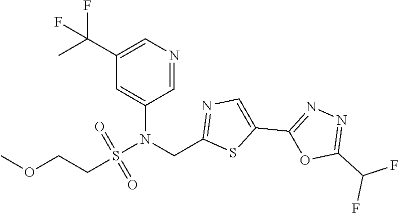 [SMILES: COCCS(=O)(=O)N(Cc1ncc(-c2nnc(C(F)F)o2)s1)c1cncc(C(C)(F)F)c1] |
| | N-[5-(1,1-difluoroethyl)pyridin-3-yl]-N-({5-[5- |
| | (difluoromethyl)-1,3,4-oxadiazol-2-yl]-1,3-thiazol-2-yl} |
| | methyl)-2-methoxyethane-1-sulfonamide |
| I-160 | 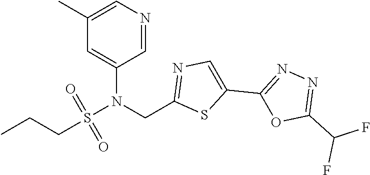 [SMILES: CCCS(=O)(=O)N(Cc1ncc(-c2nnc(C(F)F)o2)s1)c1cncc(C)c1] |
| | N-({5-[5-(difluoromethyl)-1,3,4-oxadiazol-2-yl]-1,3-thiazol-2- |
| | yl}methyl)-N-(5-methylpyridin-3-yl)propane-1-sulfonamide |
| I-161 | 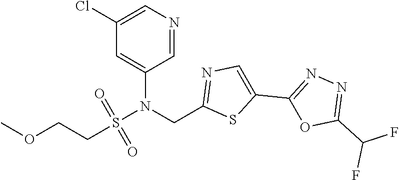 [SMILES: COCCS(=O)(=O)N(Cc1ncc(-c2nnc(C(F)F)o2)s1)c1cncc(Cl)c1] |
| | N-(5-chloropyridin-3-yl)-N-({5-[5-(difluoromethyl)-1,3,4- |
| | oxadiazol-2-yl]-1,3-thiazol-2-yl}methyl)-2-methoxyethane- |
| | 1-sulfonamide |
| I-162 | 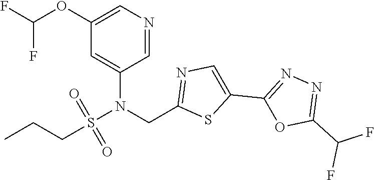 [SMILES: CCCS(=O)(=O)N(Cc1ncc(-c2nnc(C(F)F)o2)s1)c1cncc(OC(F)F)c1] |
| | N-[5-(difluoromethoxy)pyridin-3-yl]-N-({5-[5- |
| | (difluoromethyl)-1,3,4-oxadiazol-2-yl]- |
| | 1,3-thiazol-2-yl}methyl)propane-1-sulfonamide |
| I-163 | 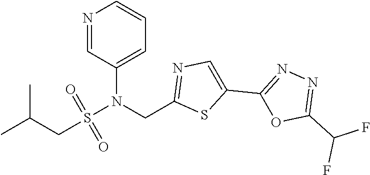 [SMILES: CC(C)CS(=O)(=O)N(Cc1ncc(-c2nnc(C(F)F)o2)s1)c1cccnc1] |
| | N-({5-[5-(difluoromethyl)-1,3,4-oxadiazol-2-yl]-1,3-thiazol-2- |
| | yl}methyl)-2-methyl-N-(pyridin-3-yl)propane-1-sulfonamide |
| I-164 | 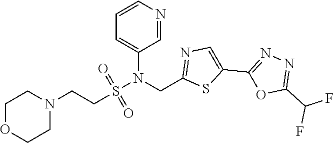 [SMILES: O=S(=O)(CCN1CCOCC1)N(Cc1ncc(-c2nnc(C(F)F)o2)s1)c1cccnc1] |
| | N-({5-[5-(difluoromethyl)-1,3,4-oxadiazol-2-yl]-1,3- |
| | thiazol-2-yl}methyl)-2-(morpholin-4-yl)-N-(pyridin- |
| | 3-yl)ethane-1-sulfonamide |
| I-165 | 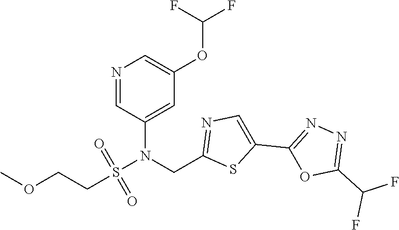 [SMILES: COCCS(=O)(=O)N(Cc1ncc(-c2nnc(C(F)F)o2)s1)c1cncc(OC(F)F)c1] |
| | N-[5-(difluoromethoxy)pyridin-3-yl]-N-({5-[5-(difluoromethyl)- |
| | 1,3,4-oxadiazol-2-yl]-1,3-thiazol-2-yl}methyl)-2- |
| | methoxyethane-1-sulfonamide |
| I-166 | 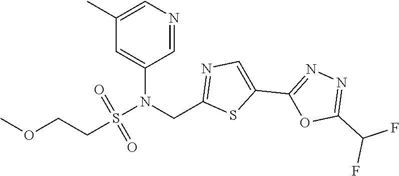 [SMILES: COCCS(=O)(=O)N(Cc1ncc(-c2nnc(C(F)F)o2)s1)c1cncc(C)c1] |
| | N-({5-[5-(difluoromethyl)-1,3,4-oxadiazol-2-yl]- |
| | 1,3-thiazol-2-yl}methyl)-2-methoxy-N-(5-methylpyridin-3-yl) |
| | ethane-1-sulfonamide |
| I-167 | 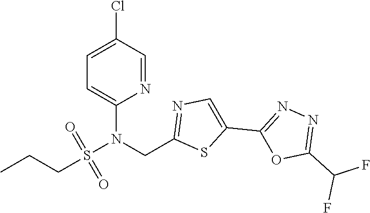 [SMILES: CCCS(=O)(=O)N(Cc1ncc(-c2nnc(C(F)F)o2)s1)c1ccc(Cl)cn1] |
| | N-(5-chloropyridin-2-yl)-N-({5-[5-(difluoromethyl)-1,3,4- |
| | oxadiazol-2-yl]-1,3-thiazol-2-yl}methyl)propane-1-sulfonamide |
| I-170 | 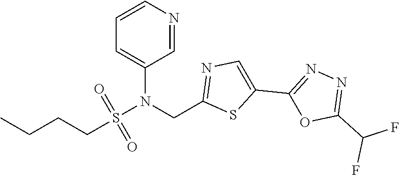 [SMILES: CCCCS(=O)(=O)N(Cc1ncc(-c2nnc(C(F)F)o2)s1)c1cccnc1] |
| | N-({5-[5-(difluoromethyl)-1,3,4-oxadiazol-2-yl]-1,3-thiazol- |
| | 2-yl}methyl)-N-(pyridin-3-yl)butane-1-sulfonamide |
| I-171 | 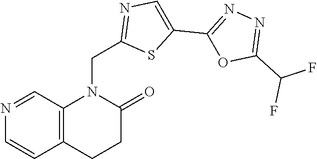 [SMILES: O=C1CCc2ccncc2N1Cc1ncc(-c2nnc(C(F)F)o2)s1] |
| | 1-({5-[5-(difluoromethyl)-1,3,4-oxadiazol-2-yl]-1,3-thiazol-2- |
| | yl}methyl)-1,2,3,4-tetrahydro-1,7-naphthyridin-2-one |
| I-172 | 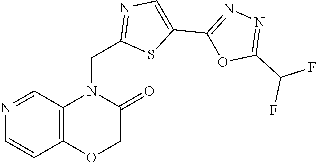 [SMILES: O=C1COc2ccncc2N1Cc1ncc(-c2nnc(C(F)F)o2)s1] |
| | 4-({5-[5-(difluoromethyl)-1,3,4-oxadiazol-2-yl]-1,3-thiazol-2- |
| | yl}methyl)-2H,3H,4H-pyrido[4,3-b][1,4]oxazin-3-one |
| I-174 | 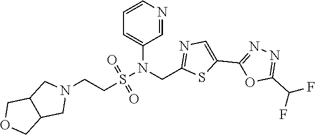 [SMILES: O=S(=O)(CCN1CC2COCC2C1)N(Cc1ncc(-c2nnc(C(F)F)o2)s1)c1cccnc1] |
| | N-((5-(5-(difluoromethyl)-1,3,4-oxadiazol-2-yl)thiazol- |
| | 2-yl)methyl)-N-(pyridin-3-yl)-2-(tetrahydro-1H-furo[3,4- |
| | c]pyrrol-5(3H)-yl)ethane-1-sulfonamide |
| I-177 | 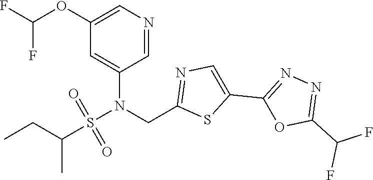 [SMILES: CCC(C)S(=O)(=O)N(Cc1ncc(-c2nnc(C(F)F)o2)s1)c1cncc(OC(F)F)c1] |
| | N-[5-(difluoromethoxy)pyridin-3-yl]-N-({5-[5- |
| | (difluoromethyl)-1,3,4-oxadiazol-2-yl]-1,3-thiazol-2-yl} |
| | methyl)butane-2-sulfonamide |
| I-179 | 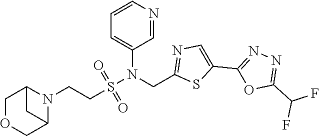 [SMILES: O=S(=O)(CCN1C2COCC1C2)N(Cc1ncc(-c2nnc(C(F)F)o2)s1)c1cccnc1] |
| | N-({5-[5-(difluoromethyl)-1,3,4-oxadiazol-2-yl]-1,3-thiazol-2- |
| | yl}methyl)-2-{3-oxa-6-azabicyclo[3.1.1]heptan-6-yl}-N-(pyridin-3- |
| | yl)ethane-1-sulfonamide |
| I-180 | 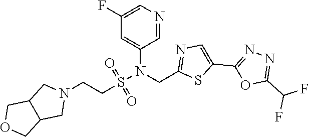 [SMILES: O=S(=O)(CCN1CC2COCC2C1)N(Cc1ncc(-c2nnc(C(F)F)o2)s1)c1cncc(F)c1] |
| | N-({5-[5-(difluoromethyl)-1,3,4-oxadiazol-2-yl]-1,3-thiazol- |
| | 2-yl}methyl)-N-(5-fluoropyridin-3-yl)-2-{hexahydro-1H- |
| | furo[3,4-c]pyrrol-5-yl}ethane-1-sulfonamide |
| I-182 | 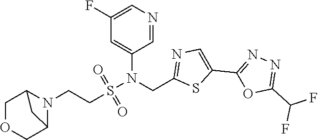 [SMILES: O=S(=O)(CCN1C2COCC1C2)N(Cc1ncc(-c2nnc(C(F)F)o2)s1)c1cncc(F)c1] |
| | N-({5-[5-(difluoromethyl)-1,3,4-oxadiazol-2-yl]-1,3-thiazol-2- |
| | yl}methyl)-N-(5-fluoropyridin-3-yl)-2-{3-oxa-6- |
| | azabicyclo[3.1.1]heptan-6-yl}ethane-1-sulfonamide |
| I-184 | 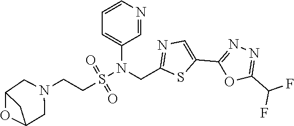 [SMILES: O=S(=O)(CCN1CC2CC(C1)O2)N(Cc1ncc(-c2nnc(C(F)F)o2)s1)c1cccnc1] |
| | N-({5-[5-(difluoromethyl)-1,3,4-oxadiazol-2-yl]-1,3-thiazol-2- |
| | yl}methyl)-2-{6-oxa-3-azabicyclo[3.1.1]heptan-3-yl}-N-(pyridin-3- |
| | yl)ethane-1-sulfonamide |
| I-186 | 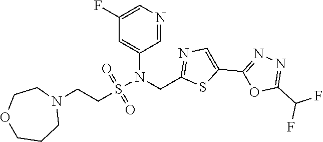 [SMILES: O=S(=O)(CCN1CCCOCC1)N(Cc1ncc(-c2nnc(C(F)F)o2)s1)c1cncc(F)c1] |
| | N-({5-[5-(difluoromethyl)-1,3,4-oxadiazol-2-yl]-1,3-thiazol-2- |
| | yl}methyl)-N-(5-fluoropyridin-3-yl)-2-(1,4-oxazepan-4-yl)ethane-1- |
| | sulfonamide |
| I-187 | 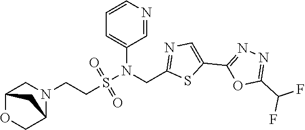 [SMILES: O=S(=O)(CCN1C[C@@H]2C[C@H]1CO2)N(Cc1ncc(-c2nnc(C(F)F)o2)s1)c1cccnc1] |
| | N-({5-[5-(difluoromethyl)-1,3,4-oxadiazol-2-yl]-1,3-thiazol-2- |
| | yl}methyl)-2-[(1S,4S)-2-oxa-5-azabicyclo[2.2.1]heptan-5-yl]-N- |
| | (pyridin-3-yl)ethane-1-sulfonamide |
| I-188 | 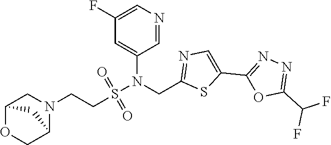 [SMILES: O=S(=O)(CCN1C[C@H]2C[C@@H]1CO2)N(Cc1ncc(-c2nnc(C(F)F)o2)s1)c1cncc(F)c1] |
| | N-({5-[5-(difluoromethyl)-1,3,4-oxadiazol-2-yl]-1,3-thiazol-2- |
| | yl}methyl)-N-(5-fluoropyridin-3-yl)-2-[(1R,4R)-2-oxa-5- |
| | azabicyclo[2.2.1]heptan-5-yl]ethane-1-sulfonamide |
| I-190 | 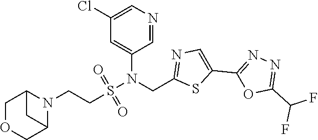 [SMILES: O=S(=O)(CCN1C2COCC1C2)N(Cc1ncc(-c2nnc(C(F)F)o2)s1)c1cncc(Cl)c1] |
| | N-(5-chloropyridin-3-yl)-N-({5-[5-(difluoromethyl)- |
| | 1,3,4-oxadiazol-2-yl]-1,3-thiazol-2-yl}methyl)-2-{3-oxa- |
| | 6-azabicyclo[3.1.1]heptan-6-yl}ethane-1-sulfonamide |
| I-191 | 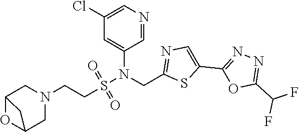 [SMILES: O=S(=O)(CCN1CC2CC(C1)O2)N(Cc1ncc(-c2nnc(C(F)F)o2)s1)c1cncc(Cl)c1] |
| | N-(5-chloropyridin-3-yl)-N-({5-[5-(difluoromethyl)-1,3,4- |
| | oxadiazol-2-yl]-1,3-thiazol-2-yl}methyl)-2-{6-oxa-3- |
| | azabicyclo[3.1.1]heptan-3-yl}ethane-1-sulfonamide |
| I-192 | 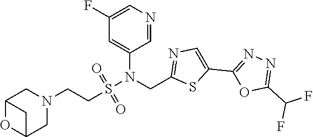 [SMILES: O=S(=O)(CCN1CC2CC(C1)O2)N(Cc1ncc(-c2nnc(C(F)F)o2)s1)c1cncc(F)c1] |
| | N-({5-[5-(difluoromethyl)-1,3,4-oxadiazol-2-yl]-1,3-thiazol-2- |
| | yl}methyl)-N-(5-fluoropyridin-3-yl)-2-{6-oxa-3- |
| | azabicyclo[3.1.1]heptan-3-yl}ethane-1-sulfonamide |
| I-193 | 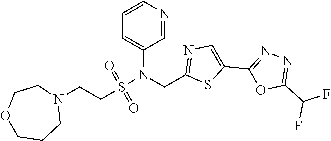 [SMILES: O=S(=O)(CCN1CCCOCC1)N(Cc1ncc(-c2nnc(C(F)F)o2)s1)c1cccnc1] |
| | N-({5-[5-(difluoromethyl)-1,3,4-oxadiazol-2-yl]- |
| | 1,3-thiazol-2-yl}methyl)-2-(1,4-oxazepan-4-yl)-N-(pyridin- |
| | 3-yl)ethane-1-sulfonamide |
| I-194 | 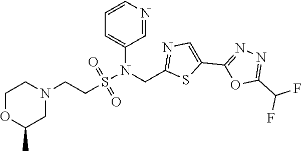 [SMILES: C[C@@H]1CN(CCS(=O)(=O)N(Cc2ncc(-c3nnc(C(F)F)o3)s2)c2cccnc2)CCO1] |
| | N-({5-[5-(difluoromethyl)-1,3,4-oxadiazol-2-yl]-1,3- |
| | thiazol-2-yl}methyl)-2-[(2R)-2-methylmorpholin-4-yl]-N- |
| | (pyridin-3-yl)ethane-1-sulfonamide |
| I-201 | 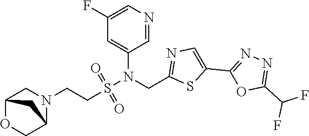 [SMILES: O=S(=O)(CCN1C[C@@H]2C[C@H]1CO2)N(Cc1ncc(-c2nnc(C(F)F)o2)s1)c1cncc(F)c1] |
| | N-({5-[5-(difluoromethyl)-1,3,4-oxadiazol-2-yl]-1,3-thiazol-2- |
| | yl}methyl)-N-(5-fluoropyridin-3-yl)-2-[(1S,4S)-2-oxa-5- |
| | azabicyclo[2.2.1]heptan-5-yl]ethane-1-sulfonamide |
| I-202 | 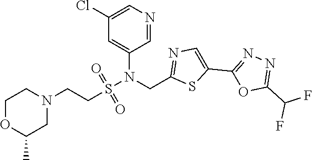 [SMILES: C[C@H]1CN(CCS(=O)(=O)N(Cc2ncc(-c3nnc(C(F)F)o3)s2)c2cncc(Cl)c2)CCO1] |
| | N-(5-chloropyridin-3-yl)-N-({5-[5-(difluoromethyl)-1,3,4- |
| | oxadiazol-2-yl]-1,3-thiazol-2-yl}methyl)-2-[(2S)-2- |
| | methylmorpholin-4-yl]ethane-1-sulfonamide |
| I-203 | 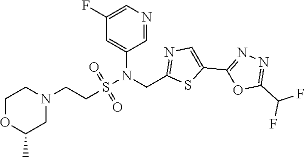 [SMILES: C[C@H]1CN(CCS(=O)(=O)N(Cc2ncc(-c3nnc(C(F)F)o3)s2)c2cncc(F)c2)CCO1] |
| | N-({5-[5-(difluoromethyl)-1,3,4-oxadiazol-2-yl]-1,3-thiazol- |
| | 2-yl}methyl)-N-(5-fluoropyridin-3-yl)-2-[(2S)-2- |
| | methylmorpholin-4-yl]ethane-1-sulfonamide |
| I-205 | 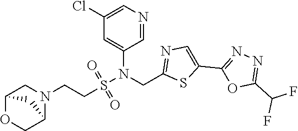 [SMILES: O=S(=O)(CCN1C[C@H]2C[C@@H]1CO2)N(Cc1ncc(-c2nnc(C(F)F)o2)s1)c1cncc(Cl)c1] |
| | N-(5-chloropyridin-3-yl)-N-({5-[5-(difluoromethyl)-1,3,4- |
| | oxadiazol-2-yl]-1,3-thiazol-2-yl}methyl)-2-[(1R,4R)-2-oxa-5- |
| | azabicyclo[2.2.1]heptan-5-yl]ethane-1-sulfonamide |
| I-206 | 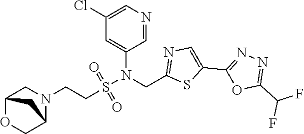 [SMILES: O=S(=O)(CCN1C[C@@H]2C[C@H]1CO2)N(Cc1ncc(-c2nnc(C(F)F)o2)s1)c1cncc(Cl)c1] |
| | N-(5-chloropyridin-3-yl)-N-({5-[5-(difluoromethyl)-1,3,4- |
| | oxadiazol-2-yl]-1,3-thiazol-2-yl}methyl)-2-[(1S,4S)-2-oxa-5- |
| | azabicyclo[2.2.1]heptan-5-yl]ethane-1-sulfonamide |
| I-207 | 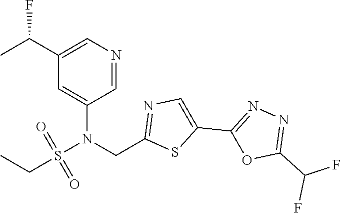 [SMILES: CCS(=O)(=O)N(Cc1ncc(-c2nnc(C(F)F)o2)s1)c1cncc([C@H](C)F)c1] |
| | N-({5-[5-(difluoromethyl)-1,3,4-oxadiazol-2-yl]-1,3-thiazol- |
| | 2-yl}methyl)-N-{5-[(1S)-1-fluoroethyl]pyridin-3-yl} |
| | ethane-1-sulfonamide |
| I-208 | 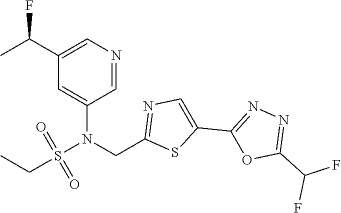 [SMILES: CCS(=O)(=O)N(Cc1ncc(-c2nnc(C(F)F)o2)s1)c1cncc([C@@H](C)F)c1] |
| | N-({5-[5-(difluoromethyl)-1,3,4-oxadiazol-2-yl]-1,3- |
| | thiazol-2-yl}methyl)-N-{5-[(1R)-1-fluoroethyl]pyridin- |
| | 3-yl}ethane-1-sulfonamide |
| I-211 | 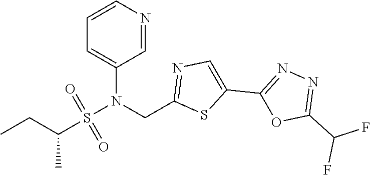 [SMILES: CC[C@@H](C)S(=O)(=O)N(Cc1ncc(-c2nnc(C(F)F)o2)s1)c1cccnc1] |
| | (2R)-N-({5-[5-(difluoromethyl)-1,3,4-oxadiazol-2-yl]-1,3- |
| | thiazol-2-yl}methyl)-N-(pyridin-3-yl)butane-2-sulfonamide |
| I-212 | 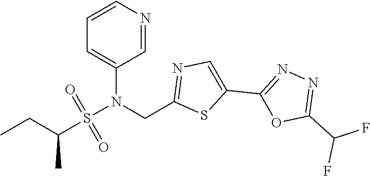 [SMILES: CC[C@H](C)S(=O)(=O)N(Cc1ncc(-c2nnc(C(F)F)o2)s1)c1cccnc1] |
| | (2S)-N-({5-[5-(difluoromethyl)-1,3,4-oxadiazol-2-yl]-1,3-thiazol-2- |
| | yl}methyl)-N-(pyridin-3-yl)butane-2-sulfonamide |
| I-214 | 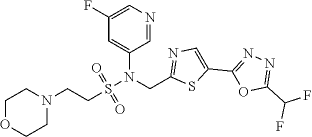 [SMILES: O=S(=O)(CCN1CCOCC1)N(Cc1ncc(-c2nnc(C(F)F)o2)s1)c1cncc(F)c1] |
| | N-({5-[5-(difluoromethyl)-1,3,4-oxadiazol-2-yl]-1,3-thiazol-2- |
| | yl}methyl)-N-(5-fluoropyridin-3-yl)-2-(morpholin-4-yl)ethane-1- |
| | sulfonamide |
| I-215 | 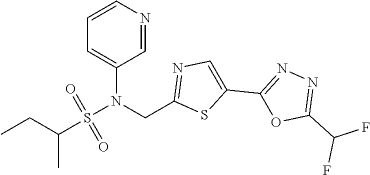 [SMILES: CCC(C)S(=O)(=O)N(Cc1ncc(-c2nnc(C(F)F)o2)s1)c1cccnc1] |
| | N-({5-[5-(difluoromethyl)-1,3,4-oxadiazol-2-yl]-1,3-thiazol-2- |
| | yl}methyl)-N-(pyridin-3-yl)butane-2-sulfonamide |
| I-216 | 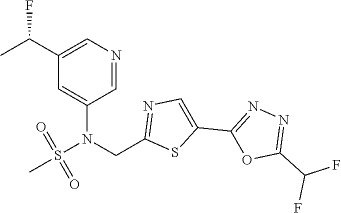 [SMILES: C[C@H](F)c1cncc(N(Cc2ncc(-c3nnc(C(F)F)o3)s2)S(C)(=O)=O)c1] |
| | N-({5-[5-(difluoromethyl)-1,3,4-oxadiazol-2-yl]- |
| | 1,3-thiazol-2-yl}methyl)-N-{5-[(1S)-1-fluoroethyl]pyridin- |
| | 3-yl}methanesulfonamide |
| I-217 | 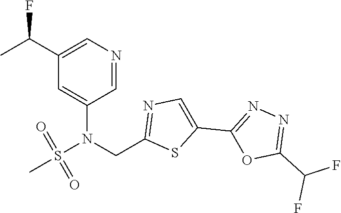 [SMILES: C[C@@H](F)c1cncc(N(Cc2ncc(-c3nnc(C(F)F)o3)s2)S(C)(=O)=O)c1] |
| | N-({5-[5-(difluoromethyl)-1,3,4-oxadiazol-2-yl]-1,3- |
| | thiazol-2-yl}methyl)-N-{5-[(1R)-1-fluoroethyl]pyridin- |
| | 3-yl}methanesulfonamide |
| I-219 | 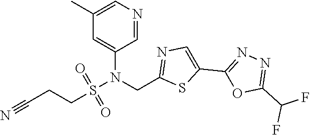 [SMILES: Cc1cncc(N(Cc2ncc(-c3nnc(C(F)F)o3)s2)S(=O)(=O)CCC#N)c1] |
| | 2-cyano-N-({5-[5-(difluoromethyl)-1,3,4-oxadiazol-2-yl]-1, |
| | 3-thiazol-2-yl}methyl)-N-(5-methylpyridin-3-yl)ethane- |
| | 1-sulfonamide |
| I-220 | 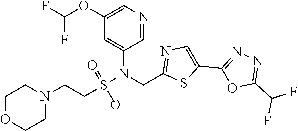 |
| | N-[5-(difluoromethoxy)pyridin-3-yl]-N-({5-[5-(difluoromethyl)- |
| | 1,3,4-oxadiazol-2-yl]-1,3-thiazol-2-yl}methyl)-2- |
| | (morpholin-4-yl)ethane-1-sulfonamide |
| I-223 | 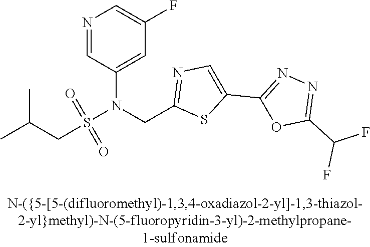 [SMILES: CC(C)CS(=O)(=O)N(Cc1ncc(-c2nnc(C(F)F)o2)s1)c1cncc(F)c1] |
| I-224 | 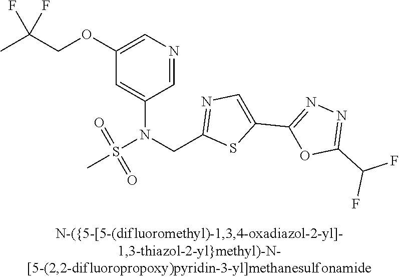 [SMILES: CC(F)(F)COc1cncc(N(Cc2ncc(-c3nnc(C(F)F)o3)s2)S(C)(=O)=O)c1] |
| I-225 | 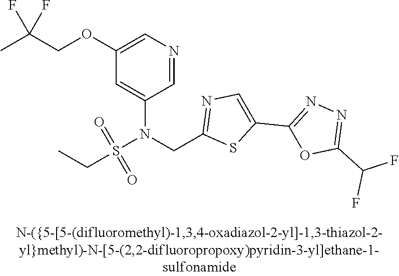 [SMILES: CCS(=O)(=O)N(Cc1ncc(-c2nnc(C(F)F)o2)s1)c1cncc(OCC(C)(F)F)c1] |
| I-227 | 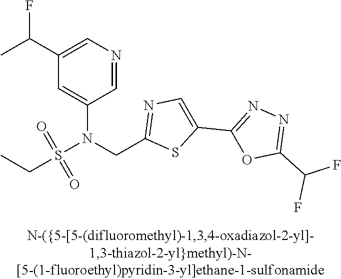 [SMILES: CCS(=O)(=O)N(Cc1ncc(-c2nnc(C(F)F)o2)s1)c1cncc(C(C)F)c1] |
| I-229 | 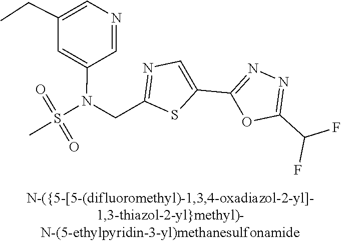 [SMILES: CCc1cncc(N(Cc2ncc(-c3nnc(C(F)F)o3)s2)S(C)(=O)=O)c1] |
| I-268 | 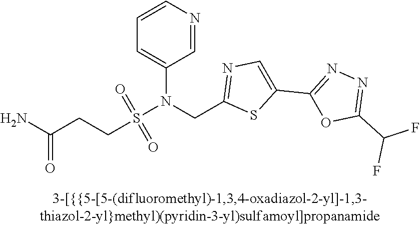 [SMILES: NC(=O)CCS(=O)(=O)N(Cc1ncc(-c2nnc(C(F)F)o2)s1)c1cccnc1] |
| I-269 | 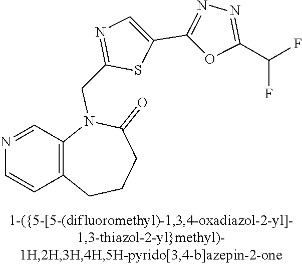 [SMILES: O=C1CCCc2ccncc2N1Cc1ncc(-c2nnc(C(F)F)o2)s1] |

8. The method of embodiment 4, wherein the HDAC6 inhibitor is a compound having the formula:

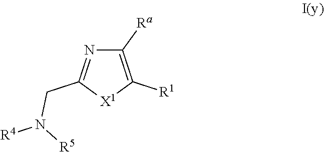

or a pharmaceutically acceptable salt thereof,
wherein:
X¹ is S;
R^{a} is selected from the group consisting of H, halogen, and C₁₋₃ alkyl;
R¹ is

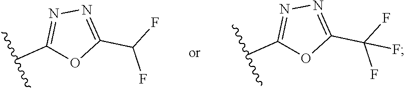 [SMILES: *c1nnc(C(F)F)o1 ; *c1nnc(C(F)(F)F)o1]

R² is selected from the group consisting of alkyl, alkoxy, and cycloalkyl, each of which is optionally substituted;
R³ is H or alkyl;
R⁴ is selected from the group consisting of alkyl, —(SO₂)R², —(SO₂)NR²R³, and —(CO)R²; and
R⁵ is aryl or heteroaryl; or R⁴ and R⁵ together with the atom to which they are attached form a heterocyclyl, each of which is optionally substituted.

9. The method of embodiment 8, wherein R^{a} is H.

10. The method of embodiment 8 or 9, wherein R¹ is

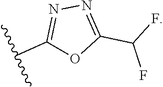 [SMILES: *c1nnc(C(F)F)o1]

11. The method of any one of embodiments 8-10, wherein R⁴ is —(SO₂)R².

12. The method of embodiment 11, wherein —(SO₂)R² is —(SO₂)alkyl, —(SO₂)alkyleneheterocyclyl, —(SO₂)haloalkyl, —(SO₂)haloalkoxy, or —(SO₂)cycloalkyl.

13. The method of any one of embodiments 8-12, wherein R⁵ is heteroaryl.

14. The method of embodiment 13, wherein the heteroaryl is a 5- to 6-membered heteroaryl

15. The method of embodiment 14, wherein the 5- to 6-membered heteroaryl is selected from the group consisting of

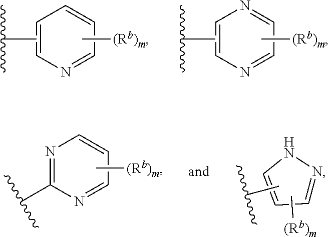

wherein R is halogen, alkyl, alkoxy, cycloalkyl, —CN, haloalkyl, or haloalkoxy; and m is 0 or 1.

16. The method of embodiment 15, wherein R^{b} is F, Cl, —CH₃, —CH₂CH₃, —CF₃, —CHF₂, —CF₂CH₃, —CN, —OCH₃, —OCH₂CH₃, —OCH(CH₃)₂, —OCF₃, —OCHF₂, —OCH₂CF₂H, and cyclopropyl.

17. The method of any one of embodiments 8-16, wherein the aryl is selected from the group consisting of phenyl, 3-chlorophenyl, 3-chloro-4-fluorophenyl, 3-trifluoromethylphenyl, 3,4-difluorophenyl, and 2,6-difluorophenyl.

18. The method of embodiment 4, wherein the HDAC6 inhibitor is a compound having Formula (Ik):

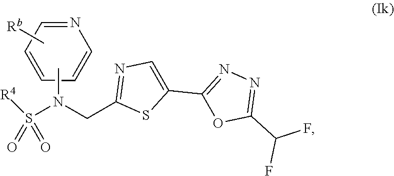

or a pharmaceutically acceptable salt thereof,
wherein:
R^{b} is H, halogen, alkyl, cycloalkyl, —CN, haloalkyl, or haloalkoxy; and
R⁴ is alkyl, alkoxy, haloalkyl, or cycloalkyl, each of which is optionally substituted.

19. The method of embodiment 18, wherein R^{b} is H, halogen, haloalkyl, or haloalkoxy.

20. The method of embodiment 18 or 19, wherein R⁴ is optionally substituted alkyl or cycloalkyl.

21. The method of embodiment 18, wherein the HDAC6 inhibitor is a compound having the structure:

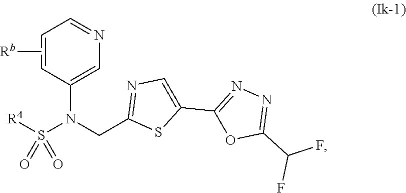

or a pharmaceutically acceptable salt thereof,
wherein:
R^{b} is H, halogen, alkyl, cycloalkyl, —CN, haloalkyl, or haloalkoxy; and
R⁴ is alkyl, alkoxy, haloalkyl, or cycloalkyl, each of which is optionally substituted.

22. The method of embodiment 21, wherein R^{b} is H, halogen, haloalkyl, or haloalkoxy.

23. The method of embodiment 21 or 22, wherein R⁴ is optionally substituted alkyl or cycloalkyl.

24. The method of embodiment 23, wherein R⁴ is alkyl.

25. The method of embodiment 18, wherein the HDAC6 inhibitor is a compound having the structure:

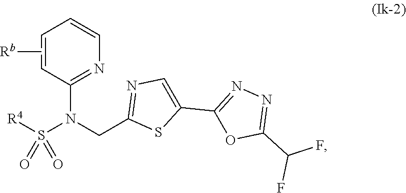

or a pharmaceutically acceptable salt thereof, wherein:
R^{b} is H, halogen, alkyl, cycloalkyl, —CN, haloalkyl, or haloalkoxy; and
R⁴ is alkyl, alkoxy, haloalkyl, or cycloalkyl, each of which is optionally substituted.

26. The method of embodiment 25, wherein R^{b} is H, halogen, haloalkyl, or haloalkoxy.

27. The method of embodiment 25 or 26, wherein R⁴ is optionally substituted alkyl.

28. The method of embodiment 8, wherein the HDAC6 inhibitor is a compound of Formula:

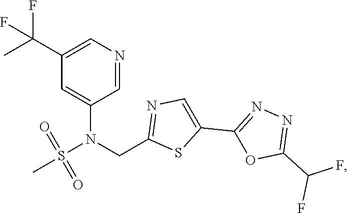 [SMILES: CC(F)(F)c1cncc(N(Cc2ncc(-c3nnc(C(F)F)o3)s2)S(C)(=O)=O)c1]

or a pharmaceutically acceptable salt thereof.

29. The method of embodiment 8, wherein the HDAC6 inhibitor is a compound of Formula:

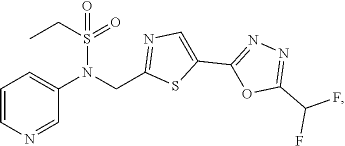 [SMILES: CCS(=O)(=O)N(Cc1ncc(-c2nnc(C(F)F)o2)s1)c1cccnc1]

or a pharmaceutically acceptable salt thereof.

30. The method of embodiment 8, wherein the HDAC6 inhibitor is a compound of Formula:

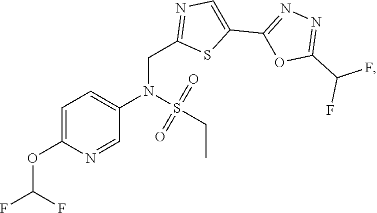 [SMILES: CCS(=O)(=O)N(Cc1ncc(-c2nnc(C(F)F)o2)s1)c1ccc(OC(F)F)nc1]

or a pharmaceutically acceptable salt thereof.

31. The method of embodiment 8, wherein the HDAC6 inhibitor is a compound of Formula:

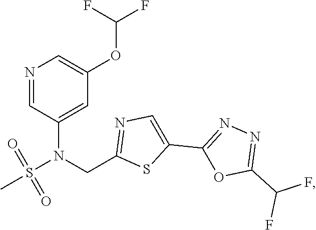 [SMILES: CS(=O)(=O)N(Cc1ncc(-c2nnc(C(F)F)o2)s1)c1cncc(OC(F)F)c1]

or a pharmaceutically acceptable salt thereof.

32. The method of embodiment 8, wherein the HDAC6 inhibitor is a compound of Formula:

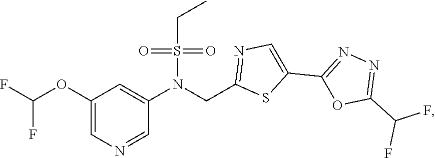 [SMILES: CCS(=O)(=O)N(Cc1ncc(-c2nnc(C(F)F)o2)s1)c1cncc(OC(F)F)c1]

or a pharmaceutically acceptable salt thereof.

33. The method of embodiment 8, wherein the HDAC6 inhibitor is a compound of Formula:

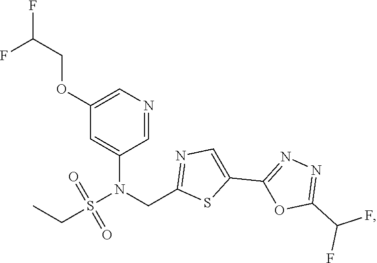 [SMILES: CCS(=O)(=O)N(Cc1ncc(-c2nnc(C(F)F)o2)s1)c1cncc(OCC(F)F)c1]

or a pharmaceutically acceptable salt thereof.

34. The method of embodiment 8, wherein the HDAC6 inhibitor is a compound of Formula:

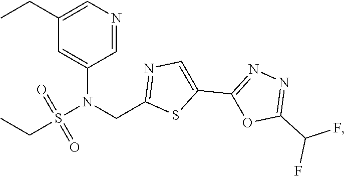 [SMILES: CCc1cncc(N(Cc2ncc(-c3nnc(C(F)F)o3)s2)S(=O)(=O)CC)c1]

or a pharmaceutically acceptable salt thereof.

35. The method of embodiment 8, wherein the HDAC6 inhibitor is a compound of Formula:

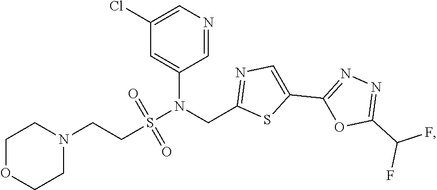 [SMILES: O=S(=O)(CCN1CCOCC1)N(Cc1ncc(-c2nnc(C(F)F)o2)s1)c1cncc(Cl)c1]

or a pharmaceutically acceptable salt thereof.

36. The method of embodiment 8, wherein the HDAC6 inhibitor is a compound of Formula:

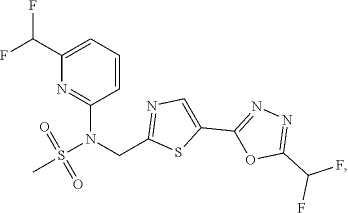 [SMILES: CS(=O)(=O)N(Cc1ncc(-c2nnc(C(F)F)o2)s1)c1cccc(C(F)F)n1]

or a pharmaceutically acceptable salt thereof.

37. The method of embodiment 8, wherein the HDAC6 inhibitor is a compound of Formula:

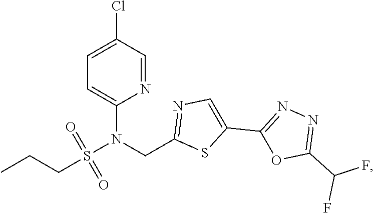 [SMILES: CCCS(=O)(=O)N(Cc1ncc(-c2nnc(C(F)F)o2)s1)c1ccc(Cl)cn1]

or a pharmaceutically acceptable salt thereof.

38. The method of embodiment 8, wherein the HDAC6 inhibitor is a compound of Formula:

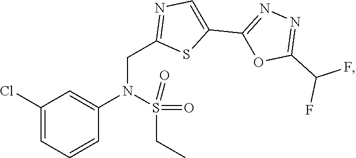 [SMILES: CCS(=O)(=O)N(Cc1ncc(-c2nnc(C(F)F)o2)s1)c1cccc(Cl)c1]

or a pharmaceutically acceptable salt thereof.

39. The method of embodiment 7, wherein the HDAC6 inhibitor is N-(3-chlorophenyl)-N-((5-(5-(difluoromethyl)-1,3,4-oxadiazol-2-yl)thiazol-2-yl)methyl)methanesulfonamide

40. The method of embodiment 7, wherein the HDAC6 inhibitor is N-(3-chlorophenyl)-N-((5-(5-(difluoromethyl)-1,3,4-oxadiazol-2-yl)thiazol-2-yl)methyl)ethanesulfonamide

41. The method of embodiment 7, wherein the HDAC6 inhibitor is N-(3-chlorophenyl)-N-((5-(5-(difluoromethyl)-1,3,4-oxadiazol-2-yl)thiazol-2-yl)methyl)cyclopropanesulfonamide

42. The method of embodiment 7, wherein the HDAC6 inhibitor is N-((5-(5-(difluoromethyl)-1,3,4-oxadiazol-2-yl)thiazol-2-yl)methyl)-N-(3,4-difluorophenyl)ethanesulfonamide

43. The method of embodiment 7, wherein the HDAC6 inhibitor is N-((5-(5-(difluoromethyl)-1,3,4-oxadiazol-2-yl)thiazol-2-yl)methyl)-N-(pyridin-3-yl)ethanesulfonamide

44. The method of embodiment 7, wherein the HDAC6 inhibitor is N-((5-(5-(difluoromethyl)-1,3,4-oxadiazol-2-yl)thiazol-2-yl)methyl)-N-phenylethanesulfonamide

45. The method of embodiment 7, wherein the HDAC6 inhibitor is N-(3-chloro-4-fluorophenyl)-N-((5-(5-(difluoromethyl)-1,3,4-oxadiazol-2-yl)thiazol-2-yl)methyl)methanesulfonamide

46. The method of embodiment 7, wherein the HDAC6 inhibitor is N-(3-chloro-4-fluorophenyl)-N-((5-(5-(difluoromethyl)-1,3,4-oxadiazol-2-yl)thiazol-2-yl)methyl)ethanesulfonamide

47. The method of embodiment 7, wherein the HDAC6 inhibitor is N-((5-(5-(difluoromethyl)-1,3,4-oxadiazol-2-yl)thiazol-2-yl)methyl)-N-(1-methyl-1H-indazol-6-yl)ethanesulfonamide

48. The method of embodiment 7, wherein the HDAC6 inhibitor is [(3-chlorophenyl)({5-[5-(difluoromethyl)-1,3,4-oxadiazol-2-yl]-1,3-thiazol-2-yl}methyl)sulfamoyl]dimethylamine

49. The method of embodiment 7, wherein the HDAC6 inhibitor is N-((5-(5-(difluoromethyl)-1,3,4-oxadiazol-2-yl)thiazol-2-yl)methyl)-N-(6-fluoropyridin-3-yl)ethanesulfonamide

50. The method of embodiment 7, wherein the HDAC6 inhibitor is N-(3-chlorophenyl)-N-((5-(5-(difluoromethyl)-1,3,4-oxadiazol-2-yl)thiazol-2-yl)methyl)thiomorpholine-4-sulfonamide 1,1-dioxide

51. The method of embodiment 7, wherein the HDAC6 inhibitor is N-(4-(1H-imidazol-1-yl)phenyl)-N-((5-(5-(difluoromethyl)-1,3,4-oxadiazol-2-yl)thiazol-2-yl)methyl)ethanesulfonamide

52. The method of embodiment 7, wherein the HDAC6 inhibitor is N-(3-chlorophenyl)-N-((5-(5-(difluoromethyl)-1,3,4-oxadiazol-2-yl)thiazol-2-yl)methyl)morpholine-4-sulfonamide

53. The method of embodiment 7, wherein the HDAC6 inhibitor is N-((5-(5-(difluoromethyl)-1,3,4-oxadiazol-2-yl)thiazol-2-yl)methyl)-N-(5-fluoropyridin-3-yl)ethanesulfonamide

54. The method of embodiment 7, wherein the HDAC6 inhibitor is N-(4-(1H-pyrazol-1-yl)phenyl)-N-((5-(5-(difluoromethyl)-1,3,4-oxadiazol-2-yl)thiazol-2-yl)methyl)ethanesulfonamide

55. The method of embodiment 7, wherein the HDAC6 inhibitor is N-(5-cyanopyridin-3-yl)-N-((5-(5-(difluoromethyl)-1,3,4-oxadiazol-2-yl)thiazol-2-yl)methyl)ethanesulfonamide

56. The method of embodiment 7, wherein the HDAC6 inhibitor is N-(5-cyclopropylpyridin-3-yl)-N-((5-(5-(difluoromethyl)-1,3,4-oxadiazol-2-yl)thiazol-2-yl)methyl)ethanesulfonamide

57. The method of embodiment 7, wherein the HDAC6 inhibitor is N-((5-(5-(difluoromethyl)-1,3,4-oxadiazol-2-yl)thiazol-2-yl)methyl)-N-(5-(trifluoromethyl)pyridin-3-yl)ethanesulfonamide

58. The method of embodiment 7, wherein the HDAC6 inhibitor is N-((5-(5-(difluoromethyl)-1,3,4-oxadiazol-2-yl)thiazol-2-yl)methyl)-N-(6-(difluoromethyl)pyridin-2-yl)ethanesulfonamide

59. The method of embodiment 7, wherein the HDAC6 inhibitor is N-((5-(5-(difluoromethyl)-1,3,4-oxadiazol-2-yl)thiazol-2-yl)methyl)-N-(4,6-dimethylpyrimidin-2-yl)ethanesulfonamide

60. The method of embodiment 7, wherein the HDAC6 inhibitor is N-((5-(5-(difluoromethyl)-1,3,4-oxadiazol-2-yl)thiazol-2-yl)methyl)-N-(1-methyl-1H-pyrazol-4-yl)ethanesulfonamide

61. The method of embodiment 7, wherein the HDAC6 inhibitor is N-(6-cyanopyridin-3-yl)-N-((5-(5-(difluoromethyl)-1,3,4-oxadiazol-2-yl)thiazol-2-yl)methyl)ethanesulfonamide

62. The method of embodiment 7, wherein the HDAC6 inhibitor is N-((5-(5-(difluoromethyl)-1,3,4-oxadiazol-2-yl)thiazol-2-yl)methyl)-N-(6-(trifluoromethyl)pyridin-2-yl)ethanesulfonamide

63. The method of embodiment 7, wherein the HDAC6 inhibitor is N-(6-cyclopropylpyridin-3-yl)-N-((5-(5-(difluoromethyl)-1,3,4-oxadiazol-2-yl)thiazol-2-yl)methyl)ethanesulfonamide

64. The method of embodiment 7, wherein the HDAC6 inhibitor is N-((5-(5-(difluoromethyl)-1,3,4-oxadiazol-2-yl)thiazol-2-yl)methyl)-N-(pyrazin-2-yl)ethanesulfonamide

65. The method of embodiment 7, wherein the HDAC6 inhibitor is N-((5-(5-(difluoromethyl)-1,3,4-oxadiazol-2-yl)thiazol-2-yl)methyl)-N-(5-fluoropyridin-2-yl)ethanesulfonamide

66. The method of embodiment 7, wherein the HDAC6 inhibitor is N-((5-(5-(difluoromethyl)-1,3,4-oxadiazol-2-yl)thiazol-2-yl)methyl)-N-(pyrazin-2-yl)cyclopropanesulfonamide

67. The method of embodiment 7, wherein the HDAC6 inhibitor is N-((5-(5-(difluoromethyl)-1,3,4-oxadiazol-2-yl)thiazol-2-yl)methyl)-N-(5-fluoropyridin-3-yl)cyclopropanesulfonamide.

68. The method of embodiment 7, wherein the HDAC6 inhibitor is N-((5-(5-(difluoromethyl)-1,3,4-oxadiazol-2-yl)thiazol-2-yl)methyl)-N-(5-fluoropyridin-3-yl)methanesulfonamide

69. The method of embodiment 7, wherein the HDAC6 inhibitor is N-(5-chloropyridin-3-yl)-N-((5-(5-(difluoromethyl)-1,3,4-oxadiazol-2-yl)thiazol-2-yl)methyl)methanesulfonamide

70. The method of embodiment 7, wherein the HDAC6 inhibitor is N-(5-(difluoromethoxy)pyridin-3-yl)-N-((5-(5-(difluoromethyl)-1,3,4-oxadiazol-2-yl)thiazol-2-yl)methyl)ethanesulfonamide

71. The method of embodiment 7, wherein the HDAC6 inhibitor is N-(6-cyanopyridin-3-yl)-N-((5-(5-(difluoromethyl)-1,3,4-oxadiazol-2-yl)thiazol-2-yl)methyl)cyclopropanesulfonamide

72. The method of embodiment 7, wherein the HDAC6 inhibitor is N-(5-chloropyridin-3-yl)-N-((5-(5-(difluoromethyl)-1,3,4-oxadiazol-2-yl)thiazol-2-yl)methyl)ethanesulfonamide

73. The method of embodiment 7, wherein the HDAC6 inhibitor is N-((5-(5-(difluoromethyl)-1,3,4-oxadiazol-2-yl)thiazol-2-yl)methyl)-1-methyl-N-(pyridin-3-yl)cyclopropane-1-sulfonamide

74. The method of embodiment 7, wherein the HDAC6 inhibitor is N-({5-[5-(difluoromethyl)-1,3,4-oxadiazol-2-yl]-1,3-thiazol-2-yl}methyl)-N-(5-fluoropyridin-2-yl)propane-1-sulfonamide

75. The method of embodiment 7, wherein the HDAC6 inhibitor is N-({5-[5-(difluoromethyl)-1,3,4-oxadiazol-2-yl]-1,3-thiazol-2-yl}methyl)-2-methoxy-N-(pyridin-3-yl)ethane-1-sulfonamide

76. The method of embodiment 7, wherein the HDAC6 inhibitor is N-({5-[5-(difluoromethyl)-1,3,4-oxadiazol-2-yl]-1,3-thiazol-2-yl}methyl)-N-(5-ethoxypyridin-3-yl)ethane-1-sulfonamide

77. The method of embodiment 7, wherein the HDAC6 inhibitor is N-(5-chloropyridin-3-yl)-N-({5-[5-(difluoromethyl)-1,3,4-oxadiazol-2-yl]-1,3-thiazol-2-yl}methyl)propane-1-sulfonamide

78. The method of embodiment 7, wherein the HDAC6 inhibitor is N-({5-[5-(difluoromethyl)-1,3,4-oxadiazol-2-yl]-1,3-thiazol-2-yl}methyl)-N-(5-fluoropyridin-3-yl)propane-1-sulfonamide

79. The method of embodiment 7, wherein the HDAC6 inhibitor is N-[5-(1,1-difluoroethyl)pyridin-3-yl]-N-({5-[5-(difluoromethyl)-1,3,4-oxadiazol-2-yl]-1,3-thiazol-2-yl}methyl)ethane-1-sulfonamide

80. The method of embodiment 7, wherein the HDAC6 inhibitor is N-(5-fluoropyridin-3-yl)-N-({5-[5-(trifluoromethyl)-1,3,4-oxadiazol-2-yl]-1,3-thiazol-2-yl}methyl)ethane-1-sulfonamide

81. The method of embodiment 7, wherein the HDAC6 inhibitor is N-(5-chloropyridin-3-yl)-N-({5-[5-(trifluoromethyl)-1,3,4-oxadiazol-2-yl]-1,3-thiazol-2-yl}methyl)ethane-1-sulfonamide

82. The method of embodiment 7, wherein the HDAC6 inhibitor is N-(pyrazin-2-yl)-N-({5-[5-(trifluoromethyl)-1,3,4-oxadiazol-2-yl]-1,3-thiazol-2-yl}methyl)ethane-1-sulfonamide

83. The method of embodiment 7, wherein the HDAC6 inhibitor is N-phenyl-N-({5-[5-(trifluoromethyl)-1,3,4-oxadiazol-2-yl]-1,3-thiazol-2-yl}methyl)ethane-1-sulfonamide

84. The method of embodiment 7, wherein the HDAC6 inhibitor is N-({5-[5-(difluoromethyl)-1,3,4-oxadiazol-2-yl]-1,3-thiazol-2-yl}methyl)-N-(5-fluoropyridin-2-yl)methanesulfonamide

85. The method of embodiment 7, wherein the HDAC6 inhibitor is N-({5-[5-(difluoromethyl)-1,3,4-oxadiazol-2-yl]-1,3-thiazol-2-yl}methyl)-N-(5-methylpyridin-3-yl)ethane-1-sulfonamide

86. The method of embodiment 7, wherein the HDAC6 inhibitor is N-[5-(2,2-difluoroethoxy)pyridin-3-yl]-N-({5-[5-(difluoromethyl)-1,3,4-oxadiazol-2-yl]-1,3-thiazol-2-yl}methyl)ethane-1-sulfonamide

87. The method of embodiment 7, wherein the HDAC6 inhibitor is N-(5-cyclopropylpyridin-3-yl)-N-({5-[5-(difluoromethyl)-1,3,4-oxadiazol-2-yl]-1,3-thiazol-2-yl}methyl)methanesulfonamide

88. The method of embodiment 7, wherein the HDAC6 inhibitor is N-({5-[5-(difluoromethyl)-1,3,4-oxadiazol-2-yl]-1,3-thiazol-2-yl}methyl)-N-[6-(difluoromethyl)pyridin-2-yl]methanesulfonamide

89. The method of embodiment 7, wherein the HDAC6 inhibitor is 3-chloro-N-({5-[5-(difluoromethyl)-1,3,4-oxadiazol-2-yl]-1,3-thiazol-2-yl}methyl)-N-(2-methoxyethyl)aniline

90. The method of embodiment 7, wherein the HDAC6 inhibitor is N-(5-cyanopyridin-3-yl)-N-({5-[5-(difluoromethyl)-1,3,4-oxadiazol-2-yl]-1,3-thiazol-2-yl}methyl)cyclopropanesulfonamide

91. The method of embodiment 7, wherein the HDAC6 inhibitor is N-[5-(difluoromethoxy)pyridin-3-yl]-N-({5-[5-(difluoromethyl)-1,3,4-oxadiazol-2-yl]-1,3-thiazol-2-yl}methyl)propane-2-sulfonamide

92. The method of embodiment 7, wherein the HDAC6 inhibitor is N-({5-[5-(difluoromethyl)-1,3,4-oxadiazol-2-yl]-1,3-thiazol-2-yl}methyl)-N-(5-ethylpyridin-3-yl)ethane-1-sulfonamide

93. The method of embodiment 7, wherein the HDAC6 inhibitor is N-[5-(difluoromethoxy)pyridin-3-yl]-N-({5-[5-(difluoromethyl)-1,3,4-oxadiazol-2-yl]-1,3-thiazol-2-yl}methyl)methanesulfonamide

94. The method of embodiment 7, wherein the HDAC6 inhibitor is N-({5-[5-(difluoromethyl)-1,3,4-oxadiazol-2-yl]-1,3-thiazol-2-yl}methyl)-N-(5-methoxypyridin-3-yl)ethane-1-sulfonamide

95. The method of embodiment 7, wherein the HDAC6 inhibitor is N-(5-cyanopyridin-3-yl)-N-({5-[5-(difluoromethyl)-1,3,4-oxadiazol-2-yl]-1,3-thiazol-2-yl}methyl)propane-2-sulfonamide

96. The method of embodiment 7, wherein the HDAC6 inhibitor is N-[5-(1,1-difluoroethyl)pyridin-3-yl]-N-({5-[5-(difluoromethyl)-1,3,4-oxadiazol-2-yl]-1,3-thiazol-2-yl}methyl)methanesulfonamide

97. The method of embodiment 7, wherein the HDAC6 inhibitor is N-(5-chloropyridin-3-yl)-N-({5-[5-(difluoromethyl)-1,3,4-oxadiazol-2-yl]-1,3-thiazol-2-yl}methyl)-2-(morpholin-4-yl)ethane-1-sulfonamide

98. The method of embodiment 7, wherein the HDAC6 inhibitor is N-[5-(difluoromethoxy)pyridin-3-yl]-N-({5-[5-(difluoromethyl)-1,3,4-oxadiazol-2-yl]-1,3-thiazol-2-yl}methyl)-2-methylpropane-1-sulfonamide

99. The method of embodiment 7, wherein the HDAC6 inhibitor is N-(5-chloropyridin-3-yl)-2-cyano-N-({5-[5-(difluoromethyl)-1,3,4-oxadiazol-2-yl]-1,3-thiazol-2-yl}methyl)ethane-1-sulfonamide

100. The method of embodiment 7, wherein the HDAC6 inhibitor is N-[5-(1,1-difluoroethyl)pyridin-3-yl]-N-({5-[5-(difluoromethyl)-1,3,4-oxadiazol-2-yl]-1,3-thiazol-2-yl}methyl)-2-methoxyethane-1-sulfonamide

101. The method of embodiment 7, wherein the HDAC6 inhibitor is N-({5-[5-(difluoromethyl)-1,3,4-oxadiazol-2-yl]-1,3-thiazol-2-yl}methyl)-N-(5-methylpyridin-3-yl)propane-1-sulfonamide

102. The method of embodiment 7, wherein the HDAC6 inhibitor is N-(5-chloropyridin-3-yl)-N-({5-[5-(difluoromethyl)-1,3,4-oxadiazol-2-yl]-1,3-thiazol-2-yl}methyl)-2-methoxyethane-1-sulfonamide

103. The method of embodiment 7, wherein the HDAC6 inhibitor is N-[5-(difluoromethoxy)pyridin-3-yl]-N-({5-[5-(difluoromethyl)-1,3,4-oxadiazol-2-yl]-1,3-thiazol-2-yl}methyl)propane-1-sulfonamide

104. The method of embodiment 7, wherein the HDAC6 inhibitor is N-({5-[5-(difluoromethyl)-1,3,4-oxadiazol-2-yl]-1,3-thiazol-2-yl}methyl)-2-methyl-N-(pyridin-3-yl)propane-1-sulfonamide

105. The method of embodiment 7, wherein the HDAC6 inhibitor is N-({5-[5-(difluoromethyl)-1,3,4-oxadiazol-2-yl]-1,3-thiazol-2-yl}methyl)-2-(morpholin-4-yl)-N-(pyridin-3-yl)ethane-1-sulfonamide

106. The method of embodiment 7, wherein the HDAC6 inhibitor is N-[5-(difluoromethoxy)pyridin-3-yl]-N-({5-[5-(difluoromethyl)-1,3,4-oxadiazol-2-yl]-1,3-thiazol-2-yl}methyl)-2-methoxyethane-1-sulfonamide

107. The method of embodiment 7, wherein the HDAC6 inhibitor is N-({5-[5-(difluoromethyl)-1,3,4-oxadiazol-2-yl]-1,3-thiazol-2-yl}methyl)-2-methoxy-N-(5-methylpyridin-3-yl)ethane-1-sulfonamide

108. The method of embodiment 7, wherein the HDAC6 inhibitor is N-(5-chloropyridin-2-yl)-N-({5-[5-(difluoromethyl)-1,3,4-oxadiazol-2-yl]-1,3-thiazol-2-yl}methyl)propane-1-sulfonamide

109. The method of embodiment 7, wherein the HDAC6 inhibitor is N-({5-[5-(difluoromethyl)-1,3,4-oxadiazol-2-yl]-1,3-thiazol-2-yl}methyl)-N-(pyridin-3-yl)butane-1-sulfonamide

110. The method of embodiment 7, wherein the HDAC6 inhibitor is 1-({5-[5-(difluoromethyl)-1,3,4-oxadiazol-2-yl]-1,3-thiazol-2-yl}methyl)-1,2,3,4-tetrahydro-1,7-naphthyridin-2-one

11I. The method of embodiment 7, wherein the HDAC6 inhibitor is 4-({5-[5-(difluoromethyl)-1,3,4-oxadiazol-2-yl]-1,3-thiazol-2-yl}methyl)-2H,3H,4H-pyrido[4,3-b][1,4]oxazin-3-one

112. The method of embodiment 7, wherein the HDAC6 inhibitor is N-((5-(5-(difluoromethyl)-1,3,4-oxadiazol-2-yl)thiazol-2-yl)methyl)-N-(pyridin-3-yl)-2-(tetrahydro-1H-furo[3,4-c]pyrrol-5(3H)-yl)ethane-1-sulfonamide

113. The method of embodiment 7, wherein the HDAC6 inhibitor is N-[5-(difluoromethoxy)pyridin-3-yl]-N-({5-[5-(difluoromethyl)-1,3,4-oxadiazol-2-yl]-1,3-thiazol-2-yl}methyl)butane-2-sulfonamide

114. The method of embodiment 7, wherein the HDAC6 inhibitor is N-({5-[5-(difluoromethyl)-1,3,4-oxadiazol-2-yl]-1,3-thiazol-2-yl}methyl)-2-{3-oxa-6-azabicyclo[3.1.1]heptan-6-yl}-N-(pyridin-3-yl)ethane-1-sulfonamide

115. The method of embodiment 7, wherein the HDAC6 inhibitor is N-({5-[5-(difluoromethyl)-1,3,4-oxadiazol-2-yl]-1,3-thiazol-2-yl}methyl)-N-(5-fluoropyridin-3-yl)-2-{hexahydro-1H-furo[3,4-c]pyrrol-5-yl}ethane-1-sulfonamide

116. The method of embodiment 7, wherein the HDAC6 inhibitor is N-({5-[5-(difluoromethyl)-1,3,4-oxadiazol-2-yl]-1,3-thiazol-2-yl}methyl)-N-(5-fluoropyridin-3-yl)-2-{3-oxa-6-azabicyclo[3.1.1]heptan-6-yl}ethane-1-sulfonamide

117. The method of embodiment 7, wherein the HDAC6 inhibitor is N-({5-[5-(difluoromethyl)-1,3,4-oxadiazol-2-yl]-1,3-thiazol-2-yl}methyl)-2-{6-oxa-3-azabicyclo[3.1.1]heptan-3-yl}-N-(pyridin-3-yl)ethane-1-sulfonamide

118. The method of embodiment 7, wherein the HDAC6 inhibitor is N-({5-[5-(difluoromethyl)-1,3,4-oxadiazol-2-yl]-1,3-thiazol-2-yl}methyl)-N-(5-fluoropyridin-3-yl)-2-(1,4-oxazepan-4-yl)ethane-1-sulfonamide

119. The method of embodiment 7, wherein the HDAC6 inhibitor is N-({5-[5-(difluoromethyl)-1,3,4-oxadiazol-2-yl]-1,3-thiazol-2-yl}methyl)-2-[(1S,4S)-2-oxa-5-azabicyclo[2.2.1]heptan-5-yl]-N-(pyridin-3-yl)ethane-1-sulfonamide

120. The method of embodiment 7, wherein the HDAC6 inhibitor is N-({5-[5-(difluoromethyl)-1,3,4-oxadiazol-2-yl]-1,3-thiazol-2-yl}methyl)-N-(5-fluoropyridin-3-yl)-2-[(1R,4R)-2-oxa-5-azabicyclo[2.2.1]heptan-5-yl]ethane-1-sulfonamide

121. The method of embodiment 7, wherein the HDAC6 inhibitor is N-(5-chloropyridin-3-yl)-N-({5-[5-(difluoromethyl)-1,3,4-oxadiazol-2-yl]-1,3-thiazol-2-yl}methyl)-2-{3-oxa-6-azabicyclo[3.1.1]heptan-6-yl}ethane-1-sulfonamide

122. The method of embodiment 7, wherein the HDAC6 inhibitor is N-(5-chloropyridin-3-yl)-N-({5-[5-(difluoromethyl)-1,3,4-oxadiazol-2-yl]-1,3-thiazol-2-yl}methyl)-2-{6-oxa-3-azabicyclo[3.1.1]heptan-3-yl}ethane-1-sulfonamide

123. The method of embodiment 7, wherein the HDAC6 inhibitor is N-({5-[5-(difluoromethyl)-1,3,4-oxadiazol-2-yl]-1,3-thiazol-2-yl}methyl)-N-(5-fluoropyridin-3-yl)-2-{6-oxa-3-azabicyclo[3.1.1]heptan-3-yl}ethane-1-sulfonamide

124. The method of claim 7, wherein the HDAC6 inhibitor is N-({5-[5-(difluoromethyl)-1,3,4-oxadiazol-2-yl]-1,3-thiazol-2-yl}methyl)-2-(1,4-oxazepan-4-yl)-N-(pyridin-3-yl)ethane-1-sulfonamide

125. The method of claim 7, wherein the HDAC6 inhibitor is N-({5-[5-(difluoromethyl)-1,3,4-oxadiazol-2-yl]-1,3-thiazol-2-yl}methyl)-2-[(2R)-2-methylmorpholin-4-yl]-N-(pyridin-3-yl)ethane-1-sulfonamide

126. The method of claim 7, wherein the HDAC6 inhibitor is N-({5-[5-(difluoromethyl)-1,3,4-oxadiazol-2-yl]-1,3-thiazol-2-yl}methyl)-N-(5-fluoropyridin-3-yl)-2-[(1S,4S)-2-oxa-5-azabicyclo[2.2.1]heptan-5-yl]ethane-1-sulfonamide

127. The method of claim 7, wherein the HDAC6 inhibitor is N-(5-chloropyridin-3-yl)-N-({5-[5-(difluoromethyl)-1,3,4-oxadiazol-2-yl]-1,3-thiazol-2-yl}methyl)-2-[(2S)-2-methylmorpholin-4-yl]ethane-1-sulfonamide

128. The method of claim 7, wherein the HDAC6 inhibitor is N-({5-[5-(difluoromethyl)-1,3,4-oxadiazol-2-yl]-1,3-thiazol-2-yl}methyl)-N-(5-fluoropyridin-3-yl)-2-[(2S)-2-methylmorpholin-4-yl]ethane-1-sulfonamide

129. The method of claim 7, wherein the HDAC6 inhibitor is N-(5-chloropyridin-3-yl)-N-({5-[5-(difluoromethyl)-1,3,4-oxadiazol-2-yl]-1,3-thiazol-2-yl}methyl)-2-[(1R,4R)-2-oxa-5-azabicyclo[2.2.1]heptan-5-yl]ethane-1-sulfonamide

130. The method of claim 7, wherein the HDAC6 inhibitor is N-(5-chloropyridin-3-yl)-N-({5-[5-(difluoromethyl)-1,3,4-oxadiazol-2-yl]-1,3-thiazol-2-yl}methyl)-2-[(1S,4S)-2-oxa-5-azabicyclo[2.2.1]heptan-5-yl]ethane-1-sulfonamide

131. The method of claim 7, wherein the HDAC6 inhibitor is N-({5-[5-(difluoromethyl)-1,3,4-oxadiazol-2-yl]-1,3-thiazol-2-yl}methyl)-N-{5-[(1S)-1-fluoroethyl]pyridin-3-yl}ethane-1-sulfonamide

132. The method of claim 7, wherein the HDAC6 inhibitor is N-({5-[5-(difluoromethyl)-1,3,4-oxadiazol-2-yl]-1,3-thiazol-2-yl}methyl)-N-{5-[(1R)-1-fluoroethyl]pyridin-3-yl}ethane-1-sulfonamide

133. The method of claim 7, wherein the HDAC6 inhibitor is (2R)—N-({5-[5-(difluoromethyl)-1,3,4-oxadiazol-2-yl]-1,3-thiazol-2-yl}methyl)-N-(pyridin-3-yl)butane-2-sulfonamide

134. The method of claim 7, wherein the HDAC6 inhibitor is (2S)—N-({5-[5-(difluoromethyl)-1,3,4-oxadiazol-2-yl]-1,3-thiazol-2-yl}methyl)-N-(pyridin-3-yl)butane-2-sulfonamide

135. The method of claim 7, wherein the HDAC6 inhibitor is N-({5-[5-(difluoromethyl)-1,3,4-oxadiazol-2-yl]-1,3-thiazol-2-yl}methyl)-N-(5-fluoropyridin-3-yl)-2-(morpholin-4-yl)ethane-1-sulfonamide

136. The method of claim 7, wherein the HDAC6 inhibitor is N-({5-[5-(difluoromethyl)-1,3,4-oxadiazol-2-yl]-1,3-thiazol-2-yl}methyl)-N-(pyridin-3-yl)butane-2-sulfonamide

137. The method of claim 7, wherein the HDAC6 inhibitor is N-({5-[5-(difluoromethyl)-1,3,4-oxadiazol-2-yl]-1,3-thiazol-2-yl}methyl)-N-{5-[(1S)-1-fluoroethyl]pyridin-3-yl}methanesulfonamide

138. The method of claim 7, wherein the HDAC6 inhibitor is N-({5-[5-(difluoromethyl)-1,3,4-oxadiazol-2-yl]-1,3-thiazol-2-yl}methyl)-N-{5-[(1R)-1-fluoroethyl]pyridin-3-yl}methanesulfonamide

139. The method of claim 7, wherein the HDAC6 inhibitor is 2-cyano-N-({5-[5-(difluoromethyl)-1,3,4-oxadiazol-2-yl]-1,3-thiazol-2-yl}methyl)-N-(5-methylpyridin-3-yl)ethane-1-sulfonamide

140. The method of claim 7, wherein the HDAC6 inhibitor is N-[5-(difluoromethoxy)pyridin-3-yl]-N-({5-[5-(difluoromethyl)-1,3,4-oxadiazol-2-yl]-1,3-thiazol-2-yl}methyl)-2-(morpholin-4-yl)ethane-1-sulfonamide

141. The method of claim 7, wherein the HDAC6 inhibitor is N-({5-[5-(difluoromethyl)-1,3,4-oxadiazol-2-yl]-1,3-thiazol-2-yl}methyl)-N-(5-fluoropyridin-3-yl)-2-methylpropane-1-sulfonamide

142. The method of embodiment 7, wherein the HDAC6 inhibitor is N-({5-[5-(difluoromethyl)-1,3,4-oxadiazol-2-yl]-1,3-thiazol-2-yl}methyl)-N-[5-(2,2-difluoropropoxy)pyridin-3-yl]methanesulfonamide

143. The method of embodiment 7, wherein the HDAC6 inhibitor is N-({5-[5-(difluoromethyl)-1,3,4-oxadiazol-2-yl]-1,3-thiazol-2-yl}methyl)-N-[5-(2,2-difluoropropoxy)pyridin-3-yl]ethane-1-sulfonamide

144. The method of embodiment 7, wherein the HDAC6 inhibitor is N-({5-[5-(difluoromethyl)-1,3,4-oxadiazol-2-yl]-1,3-thiazol-2-yl}methyl)-N-[5-(1-fluoroethyl)pyridin-3-yl]ethane-1-sulfonamide

145. The method of embodiment 7, wherein the HDAC6 inhibitor is N-({5-[5-(difluoromethyl)-1,3,4-oxadiazol-2-yl]-1,3-thiazol-2-yl}methyl)-N-(5-ethylpyridin-3-yl)methanesulfonamide

146. The method of embodiment 7, wherein the HDAC6 inhibitor is 3-[({5-[5-(difluoromethyl)-1,3,4-oxadiazol-2-yl]-1,3-thiazol-2-yl}methyl)(pyridin-3-yl)sulfamoyl]propanamide

147. The method of embodiment 7, wherein the HDAC6 inhibitor is 1-({5-[5-(difluoromethyl)-1,3,4-oxadiazol-2-yl]-1,3-thiazol-2-yl}methyl)-1H,2H,3H,4H,5H-pyrido[3,4-b]azepin-2-one

148. The method of embodiment 1, wherein the HDAC6 inhibitor is a compound of Formula (II):

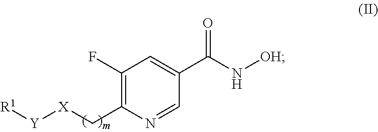

wherein
n is 0 or 1;
X is O, NR⁴, or CR⁴R^{4′};
Y is a bond, CR²R³ or S(O)₂;
R¹ is selected from the group consisting of H, amido, carbocyclyl, heterocyclyl, aryl, and heteroaryl;
R² and R³ are independently selected from the group consisting of H, halogen, alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or
R¹ and R² taken together with the carbon atom to which they are attached form a carbocyclyl or heterocyclyl; or
R² and R³ taken together with the carbon atom to which they are attached form a carbocyclyl or heterocyclyl; and
R⁴ and R^{4′} are each independently selected from the group consisting of H, alkyl, —CO₂-alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or
R⁴ and R^{4′} taken together with the carbon atom to which they are attached form a carbocyclyl or heterocyclyl;
wherein each alkyl, carbocyclyl, heterocyclyl, aryl, and heteroaryl is independently optionally substituted with one or more substituents selected from the group consisting of halogen, haloalkyl, oxo, hydroxy, alkoxy, —OCH₃, —CO₂CH₃, —C(O)NH(OH), —CH₃, morpholine, and —C(O)N-cyclopropyl.

149. The method of any one of the preceding embodiments, wherein the compound is the compound and not the pharmaceutically acceptable salt thereof.

150. The method of any one of embodiments 1-149, wherein the HDAC6 inhibitor is at least 100-fold selective against HDAC6 compared to all other isozymes of HDAC.

151. The method of any one of embodiments 1-150, wherein the dilated cardiomyopathy is familial dilated cardiomyopathy.

152. The method of any one of embodiments 1-151, wherein the dilated cardiomyopathy is dilated cardiomyopathy due to one or more BLC2-Associated Athanogene 3 (BAG3) mutations.

153. The method of any one of embodiments 1-152, wherein the subject has a deleterious or inactivating mutation in the BAG3 gene.

154. The method of embodiment 153, where the mutation in the BAG3 gene is BAG3^{E455K}

155. The method of any one of embodiments 1-151, wherein the dilated cardiomyopathy is dilated cardiomyopathy due to one or more muscle LIM protein (MLP) mutations.

156. The method of any one of embodiments 1-151, wherein the subject has a deleterious or inactivating mutation in the CSPR3 gene encoding MLP.

157. The method of any one of embodiments 1-156, wherein the subject is a human.

158. The method of any one of embodiments 1-157, wherein the method restores the ejection fraction of the subject to at least about the ejection fraction of a subject without dilated cardiomyopathy.

159. The method of any one of embodiments 1-158, wherein the method increases the ejection fraction of the subject compared to the subject's ejection fraction before treatment.

160. The method of any one of embodiments 1-159, wherein the method restores the ejection fraction of the subject to at least about 20%, at least about 20%, at least about 30%, at least about 40%, or at least about 50%.

161. The method of any one of embodiments 1-160, wherein the method increases the ejection fraction of the subject to by at least about 5%, at least about 10%, at least about 20%, at least about 30%, or at least about 40%.

162. The method of any one of embodiments 1-161, wherein the method reduces HDAC6 activity in the heart of the subject.

163. The method of any one of embodiments 1-162, wherein the method prevents heart failure in the subject.

164. The method of any one of embodiments 1-163, wherein the method reduces left ventricular internal diameter at diastole (LVIDd) in the subject.

165. The method of any one of embodiments 1-164, wherein the method reduces left ventricular internal diameter at systole (LVIDs) in the subject.

166. The method of any one of embodiments 1-165, wherein the method reduces left ventricular mass in the subject.

167. The method of any one of embodiments 1-166, wherein the administering is oral.

168. An HDAC6 inhibitor for use in a method for treating dilated cardiomyopathy.

169. The HDAC6 inhibitor of embodiment 168, wherein the HDAC6 inhibitor is any one described in embodiments 1-150.

170. A pharmaceutical composition for use in a method for treating dilated cardiomyopathy, comprising an HDAC6 inhibitor.

171. The pharmaceutical composition of embodiment 170, wherein the HDAC6 inhibitor is any one described in embodiments 1-150.

172. A kit comprising an HDAC6 inhibitor and instructions for use in a method for treating dilated cardiomyopathy.

173. The kit of embodiment 172, wherein the HDAC6 inhibitor is any one described in embodiments 1-150.

174. Use of an HDAC6 inhibitor in treating dilated cardiomyopathy.

175. The use of embodiment 174, wherein the HDAC6 inhibitor is any one described in embodiments 1-150.

### EXAMPLES

The invention is further illustrated by the following examples. The examples below are non-limiting are merely representative of various aspects of the invention. Solid and dotted wedges within the structures herein disclosed illustrate relative stereochemistry, with absolute stereochemistry depicted only when specifically, stated or delineated.

### Example 1

### SUMMARY

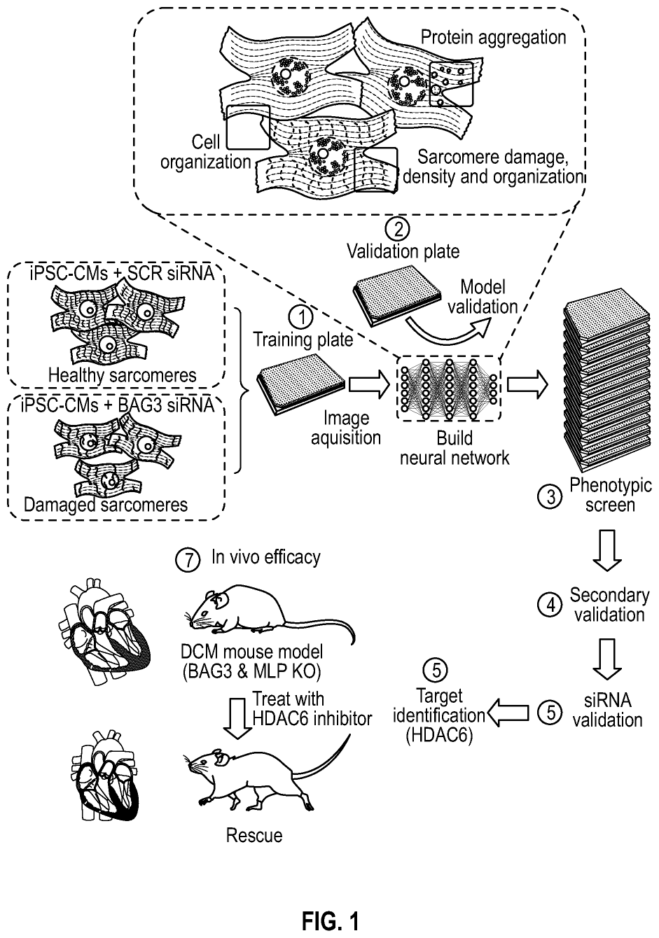

To identify candidate therapeutics, we developed an in vitro DCM model using induced pluripotent stem cell-derived cardiomyocytes (iPSC-CMs) deficient in BAG3. With these BAG3-deficient iPSC-CMs, we identified cardioprotective drugs with a phenotypic screen and deep learning (FIG. 1). Using a library of 5500 bioactive compounds and siRNA validation, we identified that inhibiting HDAC6 was cardioprotective at the sarcomere level. We translated this finding to a BAG3 cardiac-knockout (BAG3^{KO}) mouse model of DCM, showing that inhibiting HDAC6 with two isozyme-selective inhibitors (tubastatin A and TYA-018) protected heart function. TYA-018 is a compound within Formula (I), as well as within, for example, Formula I(y) and Formula (Ic).

HDAC6 inhibitors improved left ventricular ejection fraction and extended lifespan in a BAG3^{KO} mouse model of DCM. HDAC6 inhibitors also protected the microtubule network from mechanical damage, increased autophagic flux, decreased apoptosis, and reduced inflammation in the heart.

This Example demonstrates that HDAC6 inhibitors successfully treat subjects having dilated cardiomyopathy. Significantly, HDAC6 inhibitors are shown to treat dilated cardiomyopathy as measured by EF (FIGS. 11B-11C) and significantly reduced LVIDd and LVIDs (FIGS. 11D-6E).

### Results

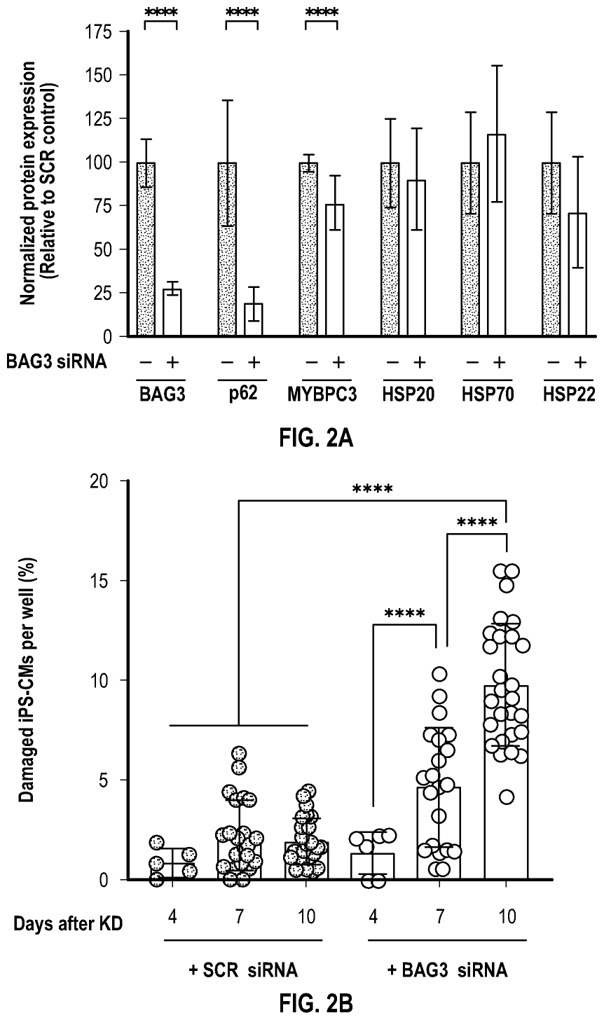

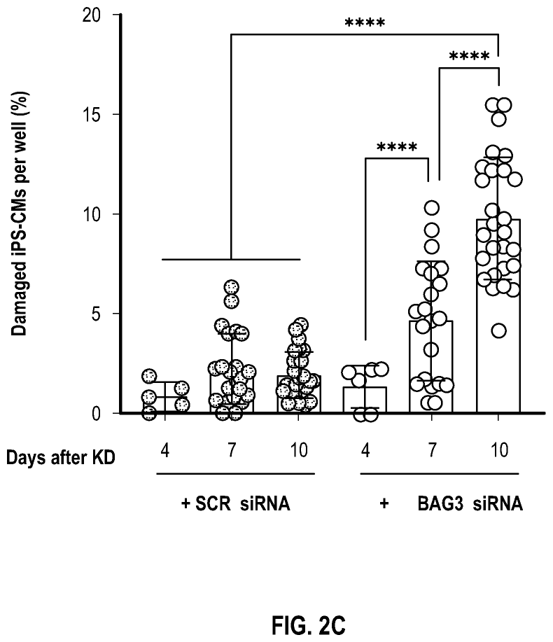

An in vitro model for DCM was transfecting cardiomyocytes derived from induced pluripotent stem cells (iPSC-CMs) with siRNA for BAG3. Reduced expression of MYBPC3 and p62 (FIG. 2A) and visually scored sarcomere damage (FIG. 2B) confirm BAG3^{KD} iPSC-CMs recapitulate the phenotype of cardiomyocytes in a subjects suffering from or at risk for DCM.

To efficiently and reproducibly quantify sarcomere damage in BAG3^{KD} iPSC-CMs, we adopted an imaging analysis method that uses deep learning (LeCun et al., 2015). We developed a 2-class deep learning model based on healthy (SCR siRNA-treated) and diseased (BAG3 siRNA-treated) iPSC-CMs.

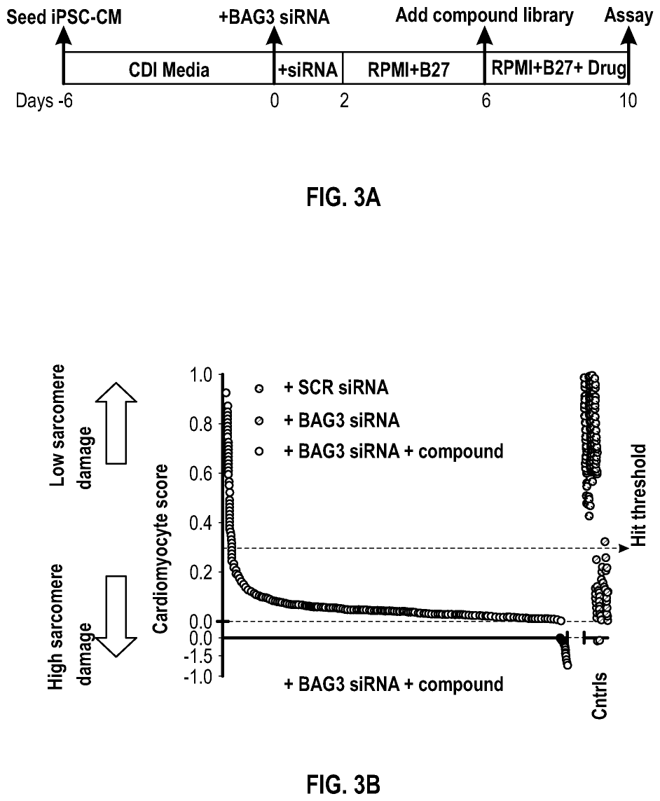

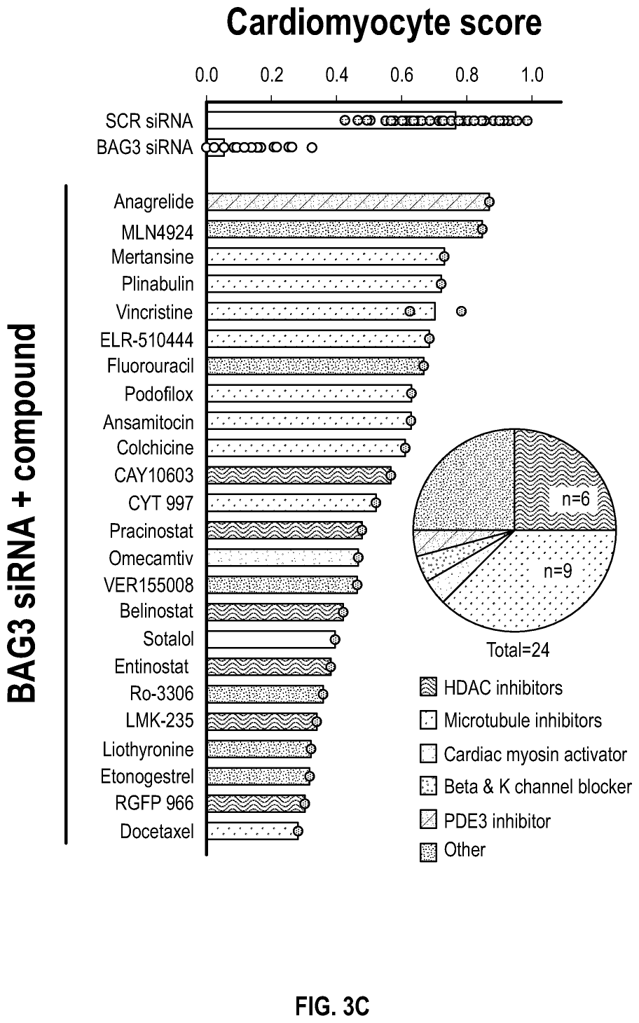

We screened 5500 bioactive compounds. iPSC-CMs were seeded, allowed to recover, treated with either SCR or BAG3 siRNA, and then treated with the bioactive compounds at a concentration of 1 Mm (FIG. 3A). Using deep learning, we determined the sarcomere score for each compound. A high sarcomere score indicated low levels of sarcomere damage, a low sarcomere score indicated high sarcomere damage, and a negative sarcomere score indicated that the compound was toxic (FIG. 3B).

We ranked screening results based on the sarcomere score and designated a hit threshold of 0.3 (false-discovery rate of 1%). Results from wells treated with either SCR or BAG3 siRNA were plotted as controls. These data showed that cells treated with SCR siRNA had a sarcomere score ranging from 0.3 to 1, whereas cells treated with BAG3 siRNA had a sarcomere score below 0.3. After manually excluding false-positive hits (due to rare staining and imaging artifacts), we grouped the top 24 hits from the screen into distinct target classes (FIG. 3C).

The top predicted cardioprotective compounds fell under two major classes: HDAC inhibitors and microtubule inhibitors. Among these, the screen identified three compounds that can be broadly classified as “standards of care” agents for cardiovascular indications: omecamtiv mecarbil (cardiac myosin activator), sotalol (beta- and K-channel blocker), and anagrelide (PDE3 inhibitor). These results further validate the translational relevance of using iPSC-CMs and deep learning to identify cardioprotective compounds in an unbiased and high-throughput manner.

The top compounds identified in the primary screen included CAY10603, a potent and selective HDAC6 inhibitor with IC₅₀ of 2 Pm and >200-fold selectivity over other HDACs.

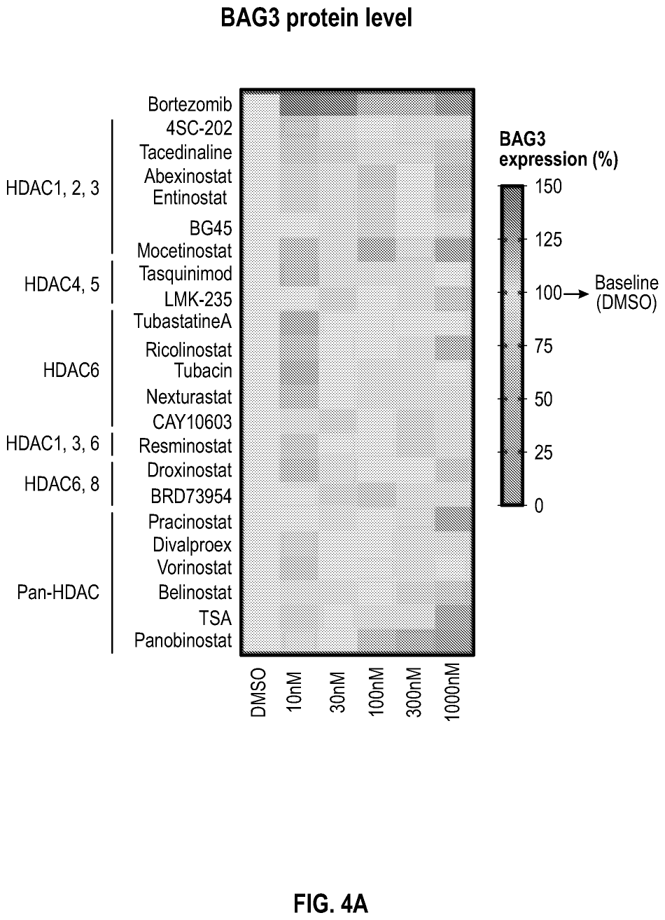

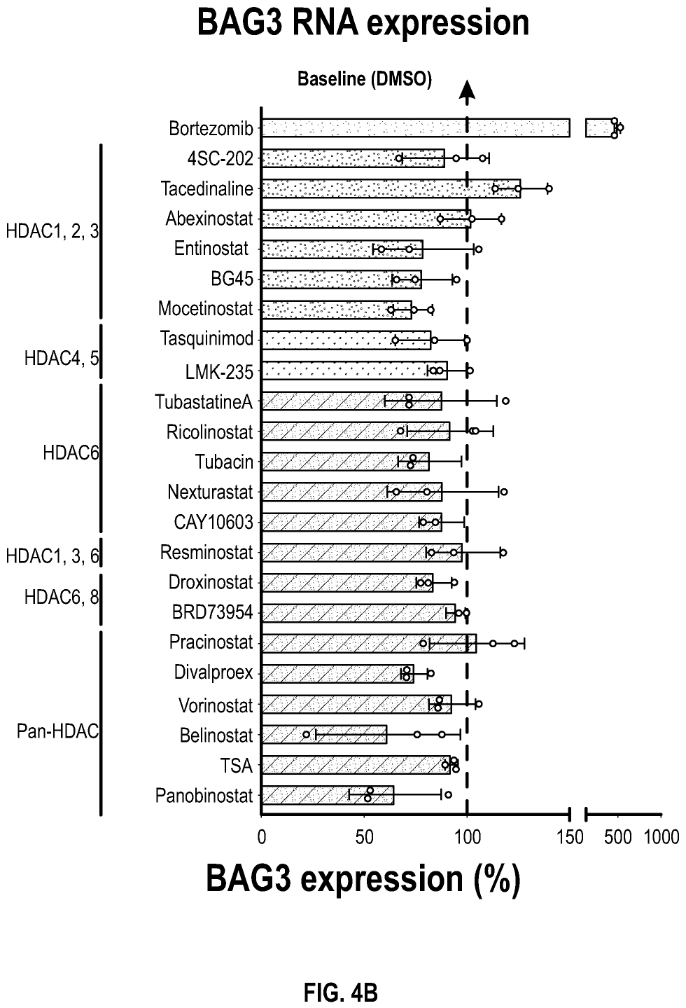

Given the abundance of hit enrichment with HDAC inhibitors, we wanted to ensure that HDAC inhibitors did not prevent sarcomere damage by increasing BAG3 expression in wild-type (WT) iPSC-CMs. To ensure we captured a broad spectrum of inhibitors, we used all HDAC inhibitors identified from the screen, as well as additional HDAC inhibitors. Using immunostaining and Qpcr, we found that none of the HDAC inhibitors increased BAG3 expression in WT iPSC-CMs (FIGS. 4A-4B). These data suggest that the HDAC inhibitors did not protect against sarcomere damage by preventing BAG3 knockdown or upregulating BAG3. Instead, they convey cardioprotection through a different mechanism.

HDAC6 Inhibition Protects Against BAG3 Loss-of-Function in iPSC-CMs

We performed a secondary validation of the top hits from the primary screen, the results of which are summarized in FIG. 5A. These results highlighted that HDAC and microtubule inhibitors are putative cardioprotective compounds. HDAC inhibitors show varying levels of polypharmacology for different HDAC isozymes. For example, class I HDACs (HDAC1, 2, 3 and 8) are predominantly located in the nucleus and target histone substrates. Inhibiting these isozymes activates global or specific gene expression programs (Haberland et al., 2009). We further interrogated all HDACs individually using siRNA to co-knockdown BAG3 and individual HDAC isoforms (HDAC1 through HDAC11). In independent studies (2-7 biological replicates), co-knockdown of HDAC6 with BAG3 prevented sarcomere damage induced by BAG3 knockdown as measured by the cardiomyocyte score (FIG. 5B). Representative immunostainings of BAG3 siRNA-treated cells showed damaged sarcomeres, which appeared significantly reduced by knockdown of HDAC6 (FIG. 5C).

We further validated these findings using siRNAs that independently target HDAC1 through HDAC11. We found that two siRNAs (1 and 3), separately and pooled, targeting HDAC6 protected against sarcomere damage in the BAG3′ model and did not affect BAG3 expression. To further confirm which HDAC is a target for tubulin, we also measured acetylated tubulin (Ac-Tubulin) levels in these knockdown studies. We found that Ac-Tubulin levels were significantly greater with knockdown of HDAC3 and HDAC6 compared to the SCR control.

### HDAC6 Inhibition or Knockout Leads to Tubulin Hyperacetylation

HDAC6 is localized in the cytoplasm (Hubbert et al., 2002; Joshi et al., 2013). In this study, we verified that HDAC6 is predominantly cytoplasmic (˜90%) in iPSC-CMs. Using clustered regularly interspaced short palindromic repeats (CRISPR)/Cas9, we generated an HDAC6 knockout (HDAC6^{KO}) iPSC line, which showed pluripotent cellular morphology. We successfully differentiated these cells to cardiomyocytes, which showed expression of sarcomeric markers (TNNT2, MYBPC3) and hyperacetylation of tubulin.

### TYA-018 is a Highly Selective HDAC6 Inhibitor

We developed a selective HDAC6 inhibitor (TYA-018) and tested its efficacy in the BAG3^{cKO} mouse model. First, we ensured high selectivity of TYA-018 using a biochemical assay and measured potency against HDAC6 and selectivity against HDAC1 (FIG. 7A). As controls, we used a pan-HDAC inhibitor (givinostat) and a well-known HDAC6-specific inhibitor (tubastatin A). In addition, we measured on-target activity of TYA-018 by measuring Ac-Tubulin in iPSC-CMs (FIGS. 7B-7C). The data suggests that TYA-018 is more potent and selective that tubastatin A. We further interrogated TYA-018 in a cell-based assay by measuring acetylated lysine on histone H3 and H4. We did not detect any off-target activity of TYA-018 on nuclear HDACs, indicating high selectivity (FIG. 7D).

We further confirmed the selectivity of TYA-018 in a full set of biochemical assays using HDAC1 through HDAC11 (FIG. 8A). We showed that TYA-018 had more than 2500-fold selectivity compared to other zinc-dependent HDACs (FIG. 8B). In addition, we profiled NAD-dependent HDACs (SIRT1 through SIRT6) and did not observe activity in the SIRT biochemical assay (data not shown). Using immunostaining, we confirmed that TYA-018 shows no evidence of off-target HDAC6 activity, as measured using acetylated lysine. With a ProBNP assay, we found that TYA-018 did not dose-dependently increase ProBNP levels, as seen with givinostat and tubastatin A, demonstrating that TYA-018 is more selective for HDAC6 than givinostat or tubastatin A (FIG. 8C). Finally, we interrogated cellular toxicity of TYA-018 in human embryonic kidney cells and showed a lethal dose (LD50) of 50 Mm.

As a final confirmation, we performed RNA-Seq on WT iPSC-CMs treated with TYA-018, givinostat, and two HDAC6-selective inhibitors (tubastatin A and ricolinostat). We showed that with increasing selectivity, we reduced the number of upregulated and downregulated transcripts in iPSC-CMs. These findings further confirm the high selectivity of TYA-018 for HDAC6, ensuring that the activity of the compound is not associated with transcriptional activation in iPSC-CMs. In addition, treatment of HDAC6 KO cells with TYA-018 does not increase the level of Ac-Tubulin.

### Cardiac-Specific Knockout of BAG3 in Mice Leads to Heart Failure

We used a cardiac-specific BAG3-knockout mouse (BAG3^{cKO}) as a model for DCM. As previously reported by Fang and colleagues (2017), this mouse model shows a rapid decline in heart function and death due to heart failure. By 5 months of age, the mice show an average ejection fraction (EF) of approximately 30% and a survival rate of approximately 50%. In addition, left ventricular internal diameter at diastole (LVIDd), left ventricular internal diameter at systole (LVIDs), and left ventricular mass were significantly greater in BAG3^{cKO} mice compared to their WT littermates. M-mode echocardiography tracings from BAG3^{cKO} mice showed a rapid decline in heart function from 1 to 5 months of age.

### HDAC6 Inhibition Prevents Progression of Heart Failure in BAG3^{cKO} Mice

To evaluate the translatability of our findings from in vitro screening to an in vivo model, we conducted an efficacy study in BAG3^{cKO} mice using a pan-HDAC inhibitor (givinostat) and an HDAC6-selective inhibitor (tubastatin A). We used both inhibitors to assess what percentage of efficacy comes from inhibiting only HDAC6 and if there are additional benefits to inhibiting other HDACs. In addition, both givinostat and tubastatin A have similar biochemical and cell-based potencies for HDAC6 inhibition (FIGS. 7A-7B).

We started administering daily doses of givinostat (30 mg/kg by oral gavage) and tubastatin A (50 mg/kg by intraperitoneal injection) when mice were 1 month old (FIG. 9A). At 1 month of age, BAG3^{cKO} mice show significantly reduced (˜13%, p<0.0001) heart function, as measured by EF, compared to WT littermate controls. Daily administration of both givinostat and tubastatin A prevented the progression of heart failure (FIGS. 9B-9E) during the 10-week period of dosing. In addition, LVIDd and LVIDs were significantly reduced in BAG3^{cKO} mice treated with givinostat (FIGS. 9F-9G) and tubastatin A (FIGS. 9H-9I).

Based on these efficacy studies, we concluded that inhibiting HDAC6 alone was sufficient to provide cardioprotection against heart failure in the BAG3^{cKO} mouse model. Also, polypharmacology associated with a pan-HDAC inhibitor did not provide further cardioprotection in these mice

### HDAC6 Inhibition Protects Against Heart Failure in BAG3^{E455K} Mice

To mimic patient-specific mutations, we used a second mouse model containing a human mutation of BAG3 (BAG3^{E455K}) (FIG. 10A). Mutations in this domain disrupt interaction with HSP70, destabilizing the chaperone complex that is essential for maintaining protein quality control and homeostasis in the cell (Fang et al., 2017). Knowing that only HDAC6 inhibition provided sufficient cardioprotection, we began a second mouse efficacy study in BAG3^{E455K} mice (Fang et al., 2017). To test if later interventions could provide protection against heart failure, we treated these mice with tubastatin A (50 mg/kg by IP) at 3 months of age. After 6 weeks of treatment, tubastatin A provided cardioprotection in BAG3^{E455K} mice as measured be EF (FIGS. 10B-10E) and reduced LVIDd and LVIDs (FIGS. 10F-10G). Most strikingly, we noticed that BAG3^{E455K} mice treated with tubastatin A had a greater lifespan (FIGS. 10H-10I). Protection against premature death due to heart failure was more pronounced in male mice.

### HDAC6 Inhibition Prevents Heart Failure in BAG3^{cKO} Mice

We tested the efficacy of the highly selective HDAC6 inhibitor TYA-018 in BAG3^{cKO} mice. In this third efficacy study, we treated mice daily with TYA-018 (15 mg/kg by oral gavage) starting at 2 months of age (FIG. 11A).

TYA-018 conferred cardioprotection in these mice during the 8-week dosing period, as measured by EF (FIGS. 11B-11C) and significantly reduced LVIDd and LVIDs (FIGS. 11D-6E).

Because TYA-018 is ultra-selective for the HDAC6 isoform (FIGS. 8A-8C), this efficacy study suggests that HDAC6 inhibition exclusively drives the efficacy.

### Treatment is Well-Tolerated in BAG3^{cKO} Mice

There was no significant effect on the weight of mice treated with TYA-018 during the 8-week period. In addition, there was no significant difference in the platelet count nor neutrophil to lymphocyte ratio in the TYA-treated BAC3^{cKO} mice compared to the vehicle control. This data suggests daily dosing of TYA-018 is well-tolerated in mice.

### Treatment Corrects Transcriptional Profile in BAG3^{cKO} Mice

We conducted principal component analysis of coding genes in hearts harvested from the three arms of the third efficacy study with TYA-018. This analysis showed a global correction of BAG3^{cKO}+TYA-018 coding genes toward their WT littermates (FIG. 11H)). RNA-Seq data from a selected number of genes is presented as a Z-score in FIG. 11I. The data showed trending correction of key sarcomere genes (MYH7, TNNI3, and MYL3) and genes that regulate mitochondrial function and metabolism (CYC1, NDUFS8, NDUFB8, PPKARG2). They also showed reduced inflammatory (IL1b, NLRP3) and apoptosis (CASP1, CAPS8) markers (FIG. 11I). RNA-Seq shows an approximately fourfold increase in NPPB expression levels in BAG3^{cKO} mice at 4 months of age compared to WT mice. TYA-018 treatment reduced NPPB levels twofold in BAG3^{cKO} mice. NPPB expression was anticorrelated with EF (FIG. 11J). Qpcr analysis further confirmed a significant reduction of NPPB levels in TYA-018 treated BAG3^{cKO} close to WT mice (FIG. 11K).

### Treatment Reduces Fragmented Nuclei and Increased LC3 Puncta in BAG3^{cKO} Mice

At 4 months of age, after 8 weeks of dosing with TYA-018, we isolated and sectioned hearts from mice of all arms of the third efficacy study. Using immunohistochemistry, we observed significantly greater fragmented nuclei in BAG3^{cKO} mice compared to their WT littermates. We confirmed these fragmented nuclei are apoptotic using TUNEL staining. TYA-018 treatment reduced the number of fragmented nuclei (p=0.073), which anticorrelated with EF in BAG3^{cKO} mice.

Additionally, by staining microtubule-associated protein light chain 3 (LC3), we observed greater LC3 puncta and a higher percentage of LC3-positive areas in hearts from BAG3^{cKO} mice treated with TYA-018. The percentage of LC3-positive areas correlated with EF in hearts from BAG3^{cKO} mice treated with TYA-018. Western blots from mouse hearts show increased levels of FLNC, PINK1 and VDAC2 and reduced levels of p62 in BAG3^{cKO} mice compared to WT mice. This finding suggests sarcomere and mitochondrial damage and impaired autophagic flux in the BAG3^{cKO} mice. TYA-018 treatment partially restored these markers in the BAG3^{cKO} back to WT levels. As expected TYA-018 treatment significantly increased Ac-Tubulin levels in the mouse hearts. Tubulin levels are significantly increased in the BAG3^{cKO} mice and the levels are unaffected with TYA-018 treatment. These data suggest that inhibiting HDAC6 with TYA-018 protects cardiac function by promoting autophagy and clearance of damaged and misfolded proteins, and by blocking apoptosis in the heart.

We next looked at five classes of drugs (15 drugs in total) that are either standard of care agents (SOC) for or are in late-stage clinical trials for cardiovascular disease to see if they had an impact on Ac-Tubulin levels. Our data shows no impact of SOC agents on Ac-Tubulin levels in iPSC-CMs nor expression of HDAC6, suggesting that cardioprotection from HDAC6 inhibition acts through an independent mechanism not covered by current SOC agents (FIGS. 12A-12B).

### HDAC6 is Increased in Ischemic Human Hearts and Various Animal Models of DCM

We analyzed biomarkers in mouse hearts and found that HDAC6 protein levels were elevated in BAG3^{cKO} mouse hearts compared to their WT littermates. We then assessed heart-tissue samples from human ischemic heart samples (FIG. 13A) and other heart failure mouse models (FIG. 13B), including pressure overload using transverse aortic constriction (TAC), angiotensin-II-induced hypertension (Ang II), and myocardial infarction (MI). HDAC6 levels were significantly elevated compared to their counterpart controls. These findings suggest that elevated HDAC6 may be a pathogenic compensatory mechanism in heart failure and that inhibiting HDAC6 may be protective in familial DCM associated with genes other than BAG3 and heart failure from other causes than DCM.

### HDAC6 Inhibition Prevents Heart Failure in a Second DCM Mouse Model (MLP^{KO})

To show that HDAC6 inhibition protects heart failure beyond the BAG3^{KO} DCM model, we tested the effect of our HDAC6 inhibitor in a second genetic model (MLP^{KO} mice). MLP (or CSRP3) is expressed in cardiac and skeletal muscle and localizes to the Z-disk (Arber et al., 1997; Knoll et al., 2010). MLP-deficient mice show sarcomere damage and myofibrillar disarray and develop dilated cardiomyopathy and heart failure (Arber et al., 1997). In this fourth efficacy study, we treated mice daily with TYA-631 (30 mg/kg by oral gavage) starting at 1.5 months of age (FIG. 13SA). TYA-631 is a highly selective HDAC6 inhibitor, as measured in cell-based and biochemical assays (FIG. S13B-S13D). TYA-631 conferred cardioprotection in these mice during the 9-week dosing period, as indicated by EF (FIGS. S13E-S13H) and reduced LVIDd and LVIDs (FIG. 6F, 6G). TYA-631 is a compound within Formula (I), as well as within, for example, Formula I(y), Formula (Ik) and Formula (Ic).

### Example 2

### Biochemical Activity and Potency of Various HDAC6 Inhibitors of Formula (I)

The compounds disclosed herein, in particular those of Formula (I), were synthesized according to methods disclosed in PCT/US2020/066439, published as WO2021127643A1, which is incorporated herein by reference in its entirety. These compounds were tested for potency against HDAC6 and selectivity against HDAC1 in a biochemical assay. A biochemical assay was adopted using a luminescent HDAC-Glo I/II assay (Promega) and measured the relative activity of HDAC6 and HDAC1 recombinant proteins. Compounds were first incubated in the presence of HDAC6 or HDAC1 separately, followed by addition of the luminescent substrate. The data was acquired using a plate reader and the biochemical IC₅₀ were calculated from the data accordingly. Data is tabulated in Table 3. From these studies, it was determined that the compounds of the present disclosure are selective inhibitors of HDAC6 over HDAC1, providing selectivity ratios from about 5 to about 30,0000.

TABLE 3
Characterization Data and HDAC6 Activity for Compounds of Formula (I).

| | | | HDAC6 |
|---|---|---|---|
| | | ¹H NMR | IC₅₀ |
| Cmpd | Structure/Name | MS (m/z) (RT) | (μM) |
| I-1 | 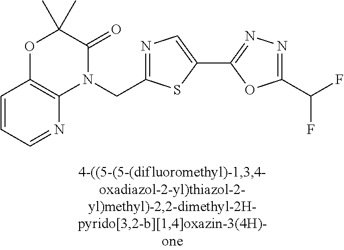 [SMILES: CC1(C)Oc2cccnc2N(Cc2ncc(-c3nnc(C(F)F)o3)s2)C1=O] | ¹H NMR (400 MHz, CDCl₃) δ 8.39 (s, 1 H), 8.01-8.05 (m, 1 H), 7.29 (dd, J = 8.1, 1.5 Hz, 1 H), 7.01 (dd, J = 8.1, 1.5 Hz, 1H), 6.89 (t, J = 51.6 Hz, 1 H), 5.70 (s, 2 H) LCMS: RT = 5.00 min, m/z = 394.0 | 0.107 |
| I-2 | 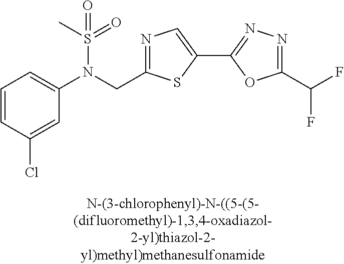 [SMILES: CS(=O)(=O)N(Cc1ncc(-c2nnc(C(F)F)o2)s1)c1cccc(Cl)c1] | ¹H NMR (400 MHz, CDCl₃) δ 8.39 (s, 1 H), 7.48 (s, 1 H), 7.32- 7.40 (m, 3 H), 6.91 (t, J = 51.6 Hz, 1 H), 5.25 (s, 2 H), 3.07 (s, 3 H) LCMS: RT= 4.83 min, m/z= 421.0 | 0.021 |
| I-3 | 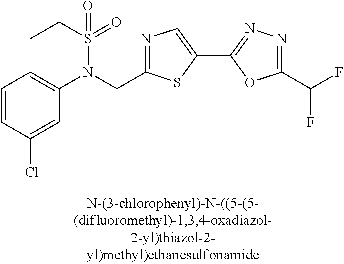 [SMILES: CCS(=O)(=O)N(Cc1ncc(-c2nnc(C(F)F)o2)s1)c1cccc(Cl)c1] | ¹H NMR (400 MHz, CDCl₃) δ 8.37 (s, 1 H), 7.48 (s, 1 H), 7.25- 7.40 (m, 3 H), 6.19 (t, J = 51.6 Hz, 1 H), 5.28 (s, 2 H), 3.18 (q, J = 7.6 Hz, 2 H), 1.44 (t, J = 7.2 Hz, 3 H) LCMS: RT = 4.90 min, m/z = 435.0 | 0.0044 |
| I-4 | 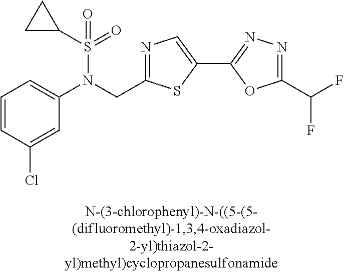 [SMILES: O=S(=O)(C1CC1)N(Cc1ncc(-c2nnc(C(F)F)o2)s1)c1cccc(Cl)c1] | ¹H NMR (400 MHz, DMSO-d₆) δ 8.53 (s, 1 H), 7.40-7.70 (m, 5 H), 5.43 (s, 2 H), 2.95-3.00 (m, 1 H), 1.00-1.05 (m, 2 H), 0.90- 0.95 (m, 2 H) LCMS: RT= 5.10 min, m/z= 447.0 | 0.0042 |
| I-5 | 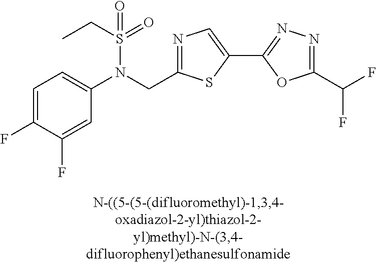 [SMILES: CCS(=O)(=O)N(Cc1ncc(-c2nnc(C(F)F)o2)s1)c1ccc(F)c(F)c1] | ¹H NMR (400 MHz, DMSO-d₆) δ 8.38 (s, 1 H), 7.30-7.40 (m, 1 H), 7.15-7.25 (m, 2 H), 6.91 (t, J = 51.2 Hz, 1 H), 5.24 (s, 2 H), 3.17 (q, J = 7.6 Hz, 2 H), 1.44 (t, J = 5.7 Hz, 3 H) LCMS: RT = 4.92 min, m/z = 437.0 | 0.016 |
| I-6 | 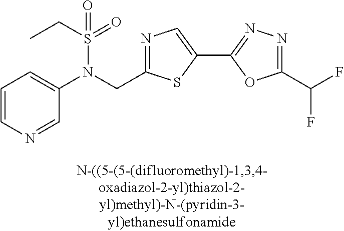 [SMILES: CCS(=O)(=O)N(Cc1ncc(-c2nnc(C(F)F)o2)s1)c1cccnc1] | ¹H NMR (400 MHz, CDCl₃) δ 8.71 (br s, 1 H), 8.58 (br s, 1 H), 8.38 (s, 1 H), 7.80-7.85 (m, 1 H), 7.31-7.35 (m 1 H), 6.91 (t, J = 51.6 Hz, 1 H), 5.30 (s, 2 H), 3.21 (q, J = 7.2 Hz, 2 H), 1.46 (t, J = 7.6 Hz, 1 H) LCMS: RT = 3.39 min, m/z = 402.0 | 0.029 |
| I-7 | 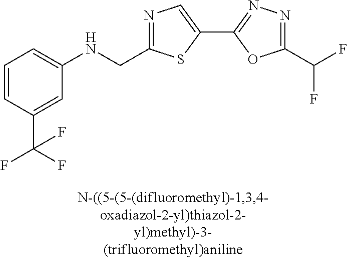 [SMILES: FC(F)c1nnc(-c2cnc(CNc3cccc(C(F)(F)F)c3)s2)o1] | ¹H NMR (400 MHz, CDCl₃) δ 8.58 (s, 1 H) 7.52 (t, J = 51.2 Hz, 1 H) 7.22-7.35 (m, 2 H) 6.89 - 6.87 (m, 3 H) 4.80 (d, J = 6.1 Hz, 2 H) LCMS: RT 5.20 min, m/z = 377.0 | 0.222 |
| I-8 | 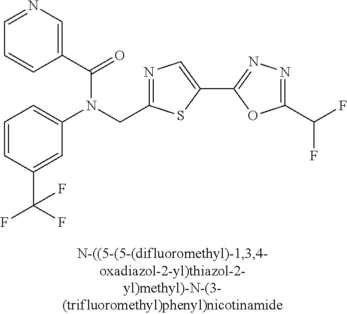 [SMILES: O=C(c1cccnc1)N(Cc1ncc(-c2nnc(C(F)F)o2)s1)c1cccc(C(F)(F)F)c1] | ¹H NMR (400 MHz, CDCl₃) δ 8.50-8.60 (m, 1 H) 8.41 (s, 1 H) 7.71 (d, J = 7.8 Hz, 1 H), 7.50 (d, J = 7.8 Hz, 1 H), 7.35-7.45 (m, 2 H), 7.25-7.30 (m, 1 H) 7.20- 7.25 (m, 1 H) 6.91 (t, J = 51.6 Hz, 1 H) 5.46 (s, 2 H) LCMS: RT = 4.22 min, m/z = 482.0 | 0.088 |
| I-9 | 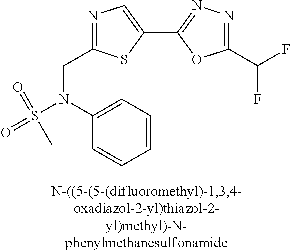 [SMILES: CS(=O)(=O)N(Cc1ncc(-c2nnc(C(F)F)o2)s1)c1ccccc1] | ¹H NMR (400 MHz, CDCl₃) δ = 8.37 (s, 1 H), 7.33-7.49 (m, 5 H), 6.90 (t, 1 H, J = 51.6 Hz) 5.27 (s, 2 H), 3.05 (s, 3 H) ppm LCMS: RT= 4.42 min, m/z = 387.0 | 0.051 |
| I-10 | 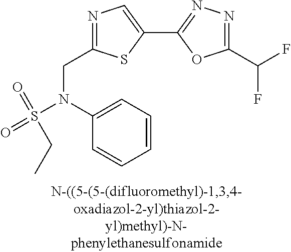 [SMILES: CCS(=O)(=O)N(Cc1ncc(-c2nnc(C(F)F)o2)s1)c1ccccc1] | ¹H NMR (400 MHz, CDCl₃) δ 8.35 (s, 1 H), 7.29-7.53 (m, 5 H), 6.90 (t, 1 H, J = 51.5 Hz), 5.30 (s, 2 H), 3.17 (q, J = 7.34 Hz, 2 H), 1.45 (t, J = 7.46 Hz, 4 H) ppm LCMS: RT = 4.65 min, m/z = 401.0 | 0.026 |
| I-12 | 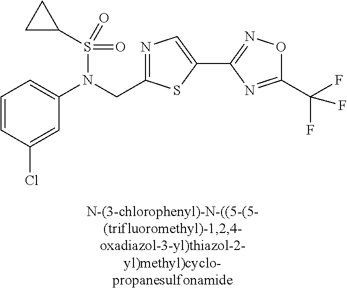 [SMILES: O=S(=O)(C1CC1)N(Cc1ncc(-c2noc(C(F)(F)F)n2)s1)c1cccc(Cl)c1] | ¹H NMR (400 MHz, chloroform- d) δ ppm 8.39 (s, 1 H) 7.51 (s, 1 H) 7.41 (br s, 1 H) 7.32 (br d, J = 3.91 Hz, 2 H) 5.27 (s, 2 H) 2.48-2.59 (m, 1H) 1.10-1.17 (m, 2 H) 0.99-1.09 (m, 2 H) LCMS: RT = 5.84 min, m/z = 465 | 0.869 |
| I-13 | 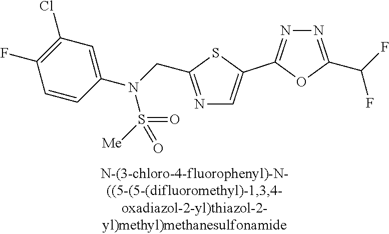 [SMILES: CS(=O)(=O)N(Cc1ncc(-c2nnc(C(F)F)o2)s1)c1ccc(F)c(Cl)c1] | ¹H NMR (400 MHz, CDCl₃) δ 8.40 (s, 1 H) 7.56 (dd, J = 6.36, 2.69 Hz, 1 H) 7.32-7.41 (m, 1 H) 7.18 (t, J = 8.56 Hz, 1 H) 6.76- 7.08 (m, 1 H) 5.22 (s, 2 H) 3.07 (s, 3 H) LCMS: RT = 4.93 min, m/z = 438.9 | 0.021 |
| I-14 | 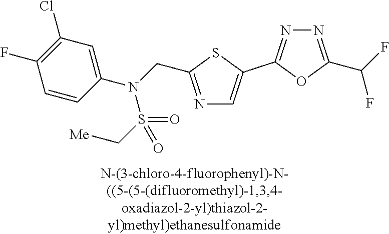 [SMILES: CCS(=O)(=O)N(Cc1ncc(-c2nnc(C(F)F)o2)s1)c1ccc(F)c(Cl)c1] | ¹H NMR (400 MHz, CDCl₃) δ 8.38 (s, 1 H) 7.54 (dd, J = 6.24, 2.32 Hz, 1 H) 7.33-7.40 (m, 1 H) 7.16 (t, J = 8.68 Hz, 1 H) 6.76- 7.06 (m, 1 H) 5.23 (s, 2 H) 3.18 (q, J = 7.34 Hz, 2 H) 1.45 (t, J = 7.34 Hz, 3 H) LCMS: RT = 5.11 min, m/z = 453 | 0.014 |
| I-15 | 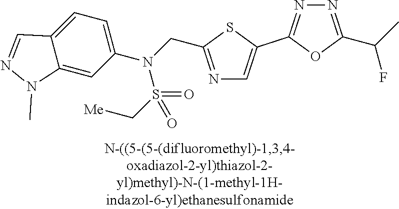 | ¹H NMR (400 MHz, CDCl₃) δ 8.36 (s, 1 H) 7.98 (d, J = 0.98 Hz, 1 H) 7.75 (d, J = 8.56 Hz, 1 H) 7.60 (s, 1 H) 7.18 (d, J = 8.31 Hz, 1H) 6.75-7.05 (m, 1 H) 5.39 (s, 2 H) 4.09 (s, 3 H) 3.19 (q, J = 7.17 Hz, 2 H) 1.46 (t, J = 7.46 Hz, 3 H) LCMS: RT = 4.43 min, m/z = 455.0. | 0.014 |
| I-16 | 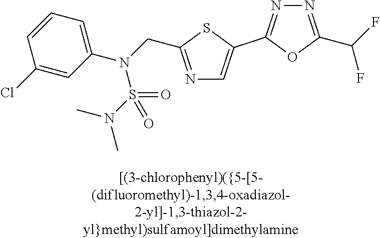 [SMILES: CN(C)S(=O)(=O)N(Cc1ncc(-c2nnc(C(F)F)o2)s1)c1cccc(Cl)c1] | ¹H NMR (400 MHz, CDCl₃) δ 8.36 (s, 1 H) 7.49 (s, 1 H) 7.28- 7.42 (m, 3 H) 6.76-6.06 (m, 1 H) 5.17 (s, 2 H) 2.82 (s, 6 H) LCMS: RT = 5.18 min, m/z = 450.0 | 0.012 |
| I-17 | 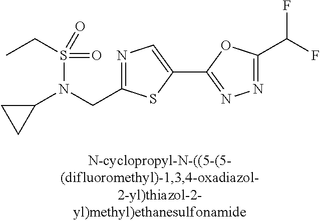 [SMILES: CCS(=O)(=O)N(Cc1ncc(-c2nnc(C(F)F)o2)s1)C1CC1] | ¹H NMR (400 MHz, chloroform-d) δ ppm 8.42 (s, 1 H) 6.74-7.07 (m, 1 H) 4.83 (s, 2 H) 3.21 (q, J = 7.50 Hz, 2 H) 2.70 (br s, 1 H) 1.41 (t, J = 7.46 Hz, 3 H) 0.83-0.95 (m, 4 H) LCMS: RT = 4.34 min, m/z = 365.0 | 1.23 |
| I-18 | 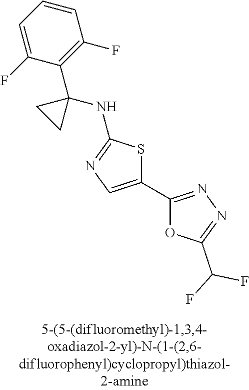 [SMILES: Fc1cccc(F)c1C1(Nc2ncc(-c3nnc(C(F)F)o3)s2)CC1] | ¹H NMR (400 MHz, DMSO-d₆) δ ppm 9.62 (br s, 1 H) 7.95 (br s, 1 H) 7.29-7.67 (overlapping m, 2 H) 7.02-7.18 (m, 2 H) 1.35 (d, J = 26.80 Hz, 4 H) LCMS: RT = 4.87 min, m/z = 371.1 | 1.95 |
| I-19 | 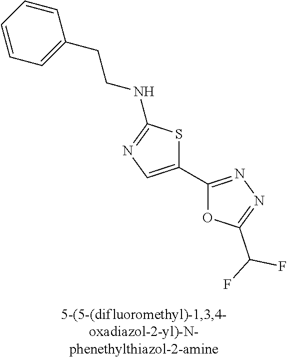 [SMILES: FC(F)c1nnc(-c2cnc(NCCc3ccccc3)s2)o1] | ¹H NMR (400 MHz, chloroform- d) δ ppm 7.86 (s, 1 H) 7.16-7.46 (m, 5 H) 6.68-7.03 (m, 1 H) 5.75 (br s, 1 H) 3.64 (br d, J = 6.11 Hz, 2 H) 3.02 (br t, J = 6.85 Hz, 2 H) LCMS: RT = 4.73 min, m/z = 323.0. | 13.6 |
| I-20 | 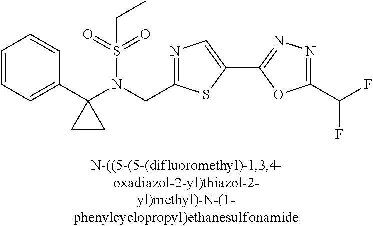 [SMILES: CCS(=O)(=O)N(Cc1ncc(-c2nnc(C(F)F)o2)s1)C1(c2ccccc2)CC1] | ¹H NMR (400 MHz, CDCl₃-d₃) δ 8.78 (s, 1 H), 8.40 (s, 1 H), 7.50 (d, J = 7.34 Hz, 2 H), 7.26-7.38 (m, 4 H), 7.04 (s, 0.25 H), 6.91 (s, 0.5 H), 6.78 (s, 0.25 H), 4.94 (s, 2 H), 2.81 (q, J = 7.58 Hz, 2 H), 1.47 (m, 2 H), 1.31-1.22 (m, 5 H). LCMS: RT = 6.12, m/z = 441.1 | 4.14 |
| I-21 | 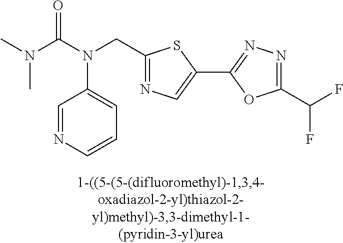 [SMILES: CN(C)C(=O)N(Cc1ncc(-c2nnc(C(F)F)o2)s1)c1cccnc1] | ¹H NMR (400 MHz, METHANOL-d4) δ ppm 9.55 (s, 1 H) 8.78 (br d, J = 5.62 Hz, 1 H) 8.52-8.59 (m, 2 H) 8.06 (br d, J = 7.83 Hz, 1 H) 7.26 (t, J = 51.2 Hz, 1 H) 6.33 (s, 2 H) 3.09 (s, 6 H). LCMS RT = 2.85 min, m/z = 381.1 | 1.2 |
| I-22 | 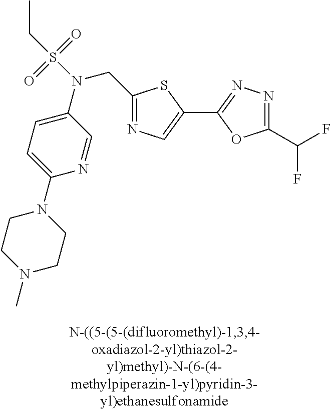 [SMILES: CCS(=O)(=O)N(Cc1ncc(-c2nnc(C(F)F)o2)s1)c1ccc(N2CCN(C)CC2)nc1] | ¹H NMR (400 MHz, CD3OD) δ 8.40 (s, 1 H), 8.15 (s, 1 H), 7.63 (m, 1 H), 7.21 (t, J = 51.2 Hz, 1H), 6.79 (m, 1 H), 5.24 (s, 2 H), 3.56 (m, 4 H), 3.25 (q, J = 7.2 Hz, 2 H), 2.51 (m, 4 H), 2.32 (s, 3 H), 1.40 (t, J = 7.2 Hz, 3 H). m/z = 500.1 | 0.143 |
| I-23 | 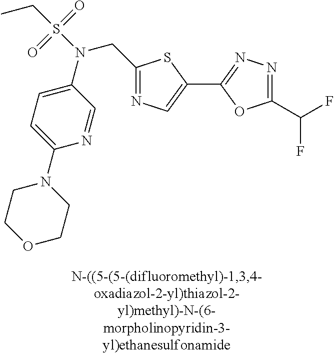 [SMILES: CCS(=O)(=O)N(Cc1ncc(-c2nnc(C(F)F)o2)s1)c1ccc(N2CCOCC2)nc1] | ¹H NMR (400 MHz, DMSO-d6) δ 8.51 (s, 1 H), 8.18 (s, 1 H), 7.69 (m, 1 H), 7.54 (t, J = 51.2 Hz, 1 H), 6.83 (m, 1 H), 5.28 (s, 1 H), 3.66 (m, 4 H), 3.44 (m, 4 H), 3.30 (q, J = 6.8 Hz, 2 H), 1.29 (t, J = 6.8 Hz, 3 H). m/z = 487.1 | 0.417 |
| I-24 | 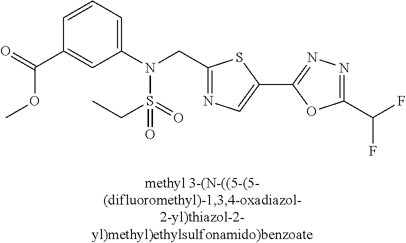 [SMILES: CCS(=O)(=O)N(Cc1ncc(-c2nnc(C(F)F)o2)s1)c1cccc(C(=O)OC)c1] | ¹H NMR (400 MHz, DMSO-d₆) δ 8.50 (s, 1 H), 8.08 (s, 1 H), 7.95- 7.83 (m, 2 H), 7.67-7.39 (m, 2 H), 5.43 (s, 2 H), 3.86 (s, 3 H), 3.45- 3.33 (m, 2 ), 1.28 (t, J = 7.21 Hz, 3 H). LCMS RT = 4.73 min, m/z = 459.1 | 0.031 |
| I-25 | 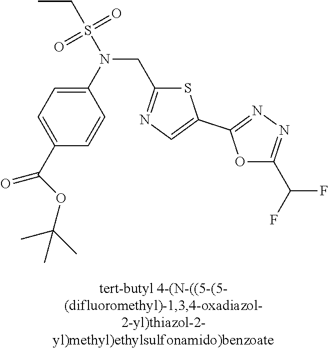 [SMILES: CCS(=O)(=O)N(Cc1ncc(-c2nnc(C(F)F)o2)s1)c1ccc(C(=O)OC(C)(C)C)cc1] | ¹H NMR (400 MHz, CDCl₃) δ 8.36 (s, 1 H), 8.01 (m, 2 H), 7.53 (m, 2 H), 6.91 (t, J = 51.6 Hz, 1 H), 5.34 (s, 2 H), 3.21 (q, J = 7.2 Hz, 2 H), 1,57 (s, 9 H), 1.43 (t, J = 7.2 Hz, 3 H). m/z = 501.1 | 0.101 |
| I-26 | 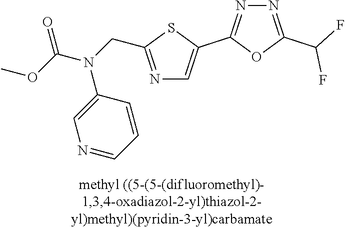 [SMILES: COC(=O)N(Cc1ncc(-c2nnc(C(F)F)o2)s1)c1cccnc1] | ¹H NMR (400 MHz, METHANOL-d4) δ ppm 9.55 (s, 1 H) 8.85 (br d, J = 5.87 Hz, 1 H) 8.57 (s, 1 H) 8.45 (br d, J = 8.80 Hz, 1 H) 8.06-8.13 (m, 1 H) 7.26 (t, J = 51.2 Hz, 1 H) 3.86 (s, 3 H). LCMS RT = 2.97 min, m/z = 368.0 | 2.5 |
| I-27 | 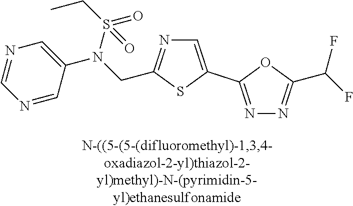 [SMILES: CCS(=O)(=O)N(Cc1ncc(-c2nnc(C(F)F)o2)s1)c1cncnc1] | ¹H NMR (400 MHz, DMSO-d₆) δ 9.12 (s, 1 H), 9.00 (s, 2 H), 8.53 (s, 1 H), 7.70-7.30 (m, 1 H), 5.50 (s, 2 H), 3.55-3.40 (m, 2 H), 1.31 (t, −7.34 Hz, 3 H). LCMS RT = 3.84 min, m/z = 403.0 | 0.050 |
| I-28 | 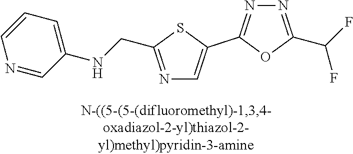 [SMILES: FC(F)c1nnc(-c2cnc(CNc3cccnc3)s2)o1] | ¹H NMR (400 MHz, METHANOL-d4) δ ppm 8.58 (s, 1 H) 8.22-8.32 (m, 2 H) 7.69- 7.78 (m, 2 H) 7.26 (t, J = 51.2 Hz, 1 H) 6.17 (s, 2 H). LCMS RT = 1.24 min, m/z = 310.1 | 1.5 |
| I-29 | 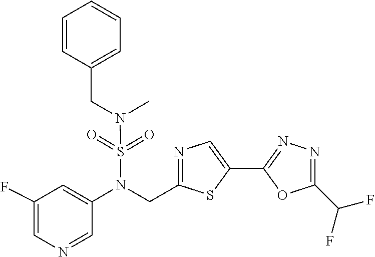 [SMILES: CN(Cc1ccccc1)S(=O)(=O)N(Cc1ncc(-c2nnc(C(F)F)o2)s1)c1cncc(F)c1] | ¹H NMR (400 MHz, CDCl₃-d₃) δ 8.55 (s, 1 H), 8.47 (s, 1 H), 8.39 (s, 1 H), 7.67 (d, J = 9.05 Hz, 1 H), 7.36-7.28 (m, 2 H), 7.19 (d, J = 6.60 Hz, 2 H), 7.04-6.77 (m, 1 H), 5.22 (s, 2 H), 4.25 (s, 2 H), 2.74 (s, 3 H). LCMS RT = 5.23 min, m/z = 511.1 | 0.041 |
| I-30 | 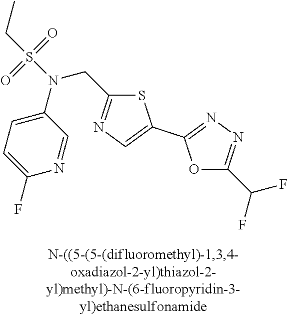 [SMILES: CCS(=O)(=O)N(Cc1ncc(-c2nnc(C(F)F)o2)s1)c1ccc(F)nc1] | ¹H NMR (400 MHz, CDCl₃) δ 8.39 (s, 1 H), 8.31 (s, 1 H), 7.94 (m, 1 H), 7.02 (m, 1 H), 6.91 (t, J = 51.6 Hz, 1 H), 5.25 (s, 2 H), 3.21 (q, J = 7.2 Hz, 2 H), 1.21 (t, J = 7.2 Hz, 3 H). m/z = 420.0 | 0.011 |
| I-31 | 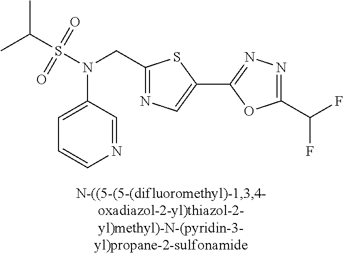 [SMILES: CC(C)S(=O)(=O)N(Cc1ncc(-c2nnc(C(F)F)o2)s1)c1cccnc1] | ¹H NMR (400 MHz, DMSO-d6) δ 8.72 (s, 1 H), 8.50 (m, 2 H), 8.02 (m, 1 H), 7.40-7.66 (m, 2 H), 5.46 (s, 2 H), 3.60 (m, 1 H), 1.34 (d, J = 6.8 Hz, 6 H). m/z = 416.1 | 0.042 |
| I-32 | 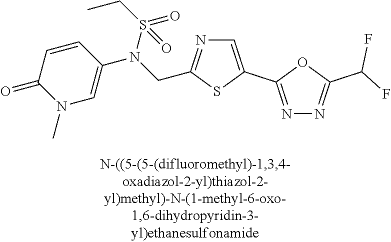 [SMILES: CCS(=O)(=O)N(Cc1ncc(-c2nnc(C(F)F)o2)s1)c1ccc(=O)n(C)c1] | ¹H NMR (400 MHz, Methanol-d₄) δ 8.47 (s, 1 H), 7.96 (s, 1 H), 7.66 (d, J = 9.54, 1 H), 7.35 (s, 0.25 H), 7.22 (s, 0.5 H), 7.09 (s, 0.25 H), 6.53 (d, J = 9.54 Hz, 1 H), 5.22 (s, 2 H), 3.56 (s, 3 H), 3.35-3.30 (m, 2 H), 1.43 (t, J = 7.34 Hz, 3 H). LCMS RT = 3.53 min, m/z = 432.1 | 0.411 |
| I-33 | 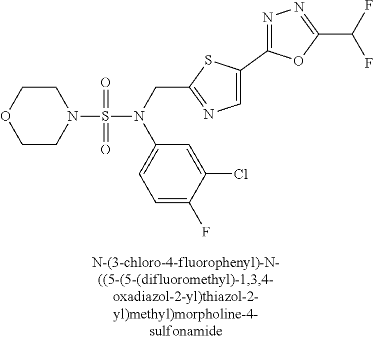 [SMILES: O=S(=O)(N1CCOCC1)N(Cc1ncc(-c2nnc(C(F)F)o2)s1)c1ccc(F)c(Cl)c1] | ¹H NMR (400 MHz, DMSO-d6) δ 8.52 (s, 1 H), 7.86-7.95 (m, 1 H), 7.35-7.69 (m, 3 H), 5.36 (s, 2 H), 3.58 (m, 4 H), 3.20 (m, 4 H). m/z = 510.0 | 0.047 |
| I-34 | 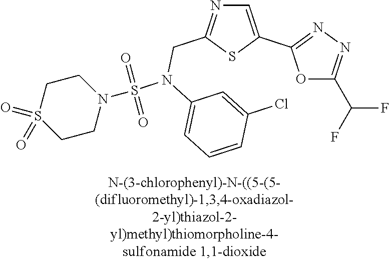 [SMILES: O=S1(=O)CCN(S(=O)(=O)N(Cc2ncc(-c3nnc(C(F)F)o3)s2)c2cccc(Cl)c2)CC1] | ¹H NMR (400 MHz, CHLOROFORM-d) δ ppm 8.41 (s, 1 H) 7.48 (s, 1 H) 7.30-7.40 (m, 3 H) 6.91 (t, J = 52.4 Hz, 1 H) 5.15 (s, 2 H) 3.73-3.82 (m, 4H) 3.05-3.13 (m, 4 H). LCMS RT = 4.83 min, m/z = 540.0 | 0.022 |
| I-35 | 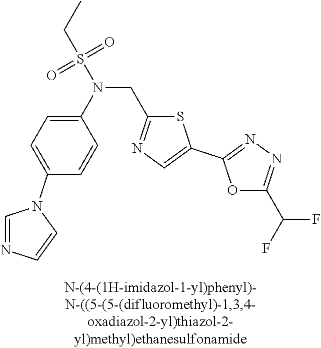 [SMILES: CCS(=O)(=O)N(Cc1ncc(-c2nnc(C(F)F)o2)s1)c1ccc(-n2ccnc2)cc1] | ¹H NMR (400 MHz, DMSO-d6) δ 8.51 (s, 1 H), 8.27 (s, 1 H), 7.69-7.76 (m, 5 H), 7.3 (t, J = 51.2 Hz, 1 H), 7.10 (s, 1 H), 5.41 (s, 2 H), 3.37 (q, J = 6.8 Hz, 2 H), 1.30 (t, J = 6.8 Hz, 3 H). m/z = 467.0 | 0.008 |
| I-36 | 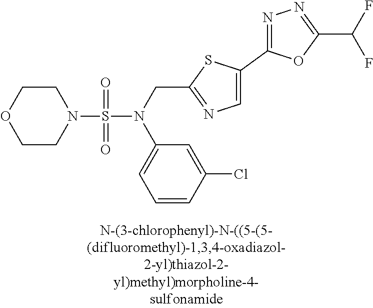 [SMILES: O=S(=O)(N1CCOCC1)N(Cc1ncc(-c2nnc(C(F)F)o2)s1)c1cccc(Cl)c1] | ¹H NMR (400 MHz, DMSO-d6) δ 8.52 (s, 1 H), 7.73 (m, 1 H), 7.54-7.58 (m, 2 H), 7.36-7.44 (m, 2 H), 5.38 (s, 2 H), 3.59 (m, 4 H), 3.18 (m, 4 H). m/z = 492.1 | 0.005 |
| I-37 | 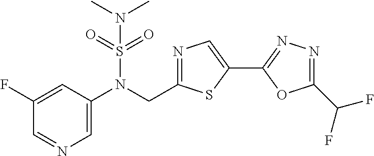 [SMILES: CN(C)S(=O)(=O)N(Cc1ncc(-c2nnc(C(F)F)o2)s1)c1cncc(F)c1] | ¹H NMR (400 MHz, DMSO-d₆) δ 8.63 (s, 1 H), 8.54 (d, J = 9. Hz, 2 H), 8.10 (d, J = 10.0 Hz, 1 H), 7.65-7.39 (m, 1 H), 5.41 (s, 2 H), 2.83 (s, 6 H). LCMS RT = 4.39 min, m/z = 435.1 | 0.013 |
| I-38 | 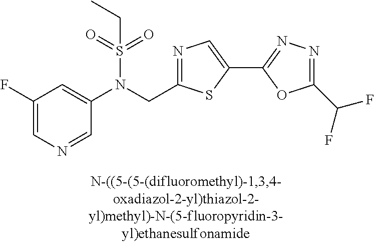 [SMILES: CCS(=O)(=O)N(Cc1ncc(-c2nnc(C(F)F)o2)s1)c1cncc(F)c1] | ¹H NMR (400 MHz, DMSO-d₆) δ 8.62-8.52 (m, H), 8.13-8.09 (m, 1 H), 7.65-7.39 (m, 1 H), 5.46 (s, 2 H), 3.55-3.45 (m, 2 H), 1.30 (t, J = 7.34 Hz, 3 H). LCMS RT = 4.32 min, m/z = 420.0 | 0.019 |
| I-39 | 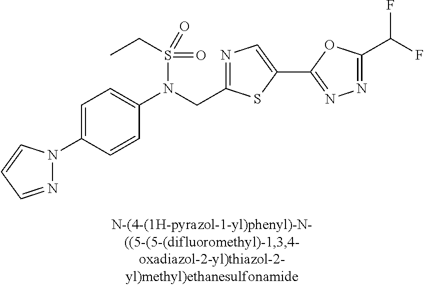 [SMILES: CCS(=O)(=O)N(Cc1ncc(-c2nnc(C(F)F)o2)s1)c1ccc(-n2cccn2)cc1] | ¹H NMR (400 MHz, CHLOROFORM-d) δ ppm 8.36 (s, 1 H) 7.90 (s, 1 H) 7.69 - 7.77 (m, 3 H) 7.55 (d, J = 8.31 Hz, 2 H) 6.90 (t, J = 52.0 Hz, 1 H) 6.48 (s, 1 H) 5.31 (s, 2 H) 3.19 (d, J = 7.34 Hz, 2 H) 1.46 (t, J = 7.46 Hz, 3 H). LCMS RT = 4.67 min, m/z = 467.0 | 0.010 |
| I-40 | 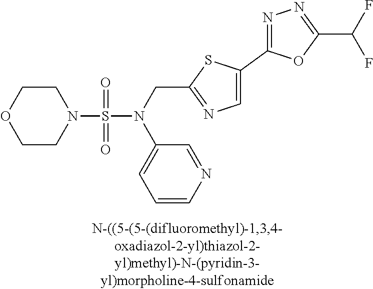 [SMILES: O=S(=O)(N1CCOCC1)N(Cc1ncc(-c2nnc(C(F)F)o2)s1)c1cccnc1] | ¹H NMR (400 MHz, CD3OD) δ 8.57 (s, 1 H), 8.45 (s, 1 H), 8.27 (m, 1 H), 7.97 (m, 1 H), 7.73 (m, 1 H), 7.22 (t, J = 51.6 Hz, 1 H), 4.88 (s, 2 H), 3.36 (m, 4 H), 3.10 (m, 4 H). m/z = 459.1 | 4.22 |
| I-43 | 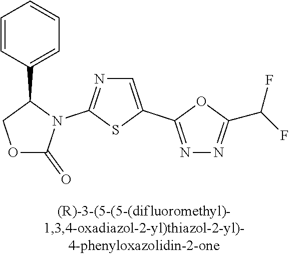 [SMILES: O=C1OC[C@@H](c2ccccc2)N1c1ncc(-c2nnc(C(F)F)o2)s1] | ¹H NMR (400 MHz, METHANOL-d4) δ = 8.07 (s, 1 H) 7.40 (s, 5 H) 7.19 (t, 1 H, J = 51.6 Hz) 5.84 (dd, J = 8.80, 3.67 Hz, 1 H) 5.03 (t, J = 8.80 Hz, 1 H) 4.48 (dd, J = 8.93, 4.03 Hz, 1 H) ppm LCMS: RT = 4.67 min, m/z = 365.1 | 0.085 |
| I-44 | 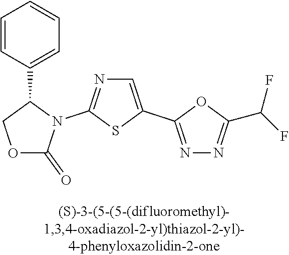 [SMILES: O=C1OC[C@H](c2ccccc2)N1c1ncc(-c2nnc(C(F)F)o2)s1] | 1H NMR (400 MHz, METHANOL-d4) δ = 8.07 (s, 1 H), 7.40 (s, 4 H), 7.33-7.38 (m, 1 H), 7.19 (t, 1 H, J = 5.6 Hz), 5.84 (dd, J = 8.56, 3.91 Hz, 1 H), 5.03 (t, J = 8.93 Hz, 1 H), 4.43-4.51 (m, 1 H) ppm LCMS: RT = 4.95 min, m/z = 365.1 | 8.97 |
| I-45 | 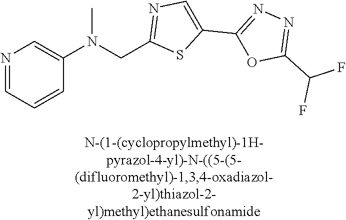 [SMILES: CN(Cc1ncc(-c2nnc(C(F)F)o2)s1)c1cccnc1] | 1H NMR (400 MHz, CD3OD) δ ppm 8.55 (s, 1 H), 8.27-8.24 (m, 2 H), 7.78-7.67 (m, 2 H), 7.23 (t, J = 51.6 Hz, 1 H), 6.15 (s, 2 H), 2.90 (s, 3 H). LCMS RT = 2.431 min, m/z = 324.0 | 1.3 |
| I-46 | 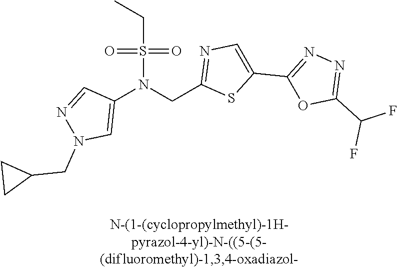 [SMILES: CCS(=O)(=O)N(Cc1ncc(-c2nnc(C(F)F)o2)s1)c1cnn(CC2CC2)c1] | 1H NMR (400 MHz, CDCl3) δ ppm 8.40 (s, 1 H), 7.69 (s, 1 H), 7.49 (s, 1 H), 6.91 (t, J = 51.6 Hz, 1 H), 5.17 (s, 2 H), 3.93 (d, J = 7.2 Hz, 2 H), 3.18 (q, J = 7.6 Hz, 2 H), 1.42 (t, J = 7.6 Hz, 3 H), 1.31-1.22 (m, 1 H), 0.70-0.62 (m, 2 H), 0.38-0.32 (m, 2 H). LCMS RT = 2.417 min, m/z = 444.9 | 0.077 |
| I-47 | 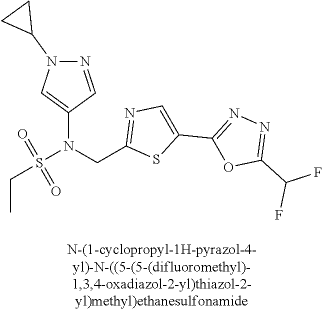 [SMILES: CCS(=O)(=O)N(Cc1ncc(-c2nnc(C(F)F)o2)s1)c1cnn(C2CC2)c1] | 1H NMR (400 MHz, CDCl3) δ ppm 8.40 (s, 1 H), 7.65 (s, 1 H), 7.46 (s, 1 H), 6.91 (t, J = 51.6 Hz, 1 H), 5.14 (s, 2 H), 3.58-3.52 (m, 1 H), 3.16 (q, J = 8.0 Hz , 2 H), 1.42 (t, J = 8.0 Hz, 3 H), 1.13- 1.00 (m, 4 H). LCMS RT = 1.223 min, m/z = 430.9 | 0.070 |
| I-48 | 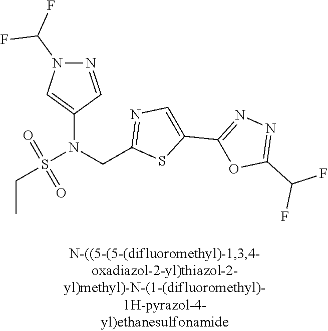 [SMILES: CCS(=O)(=O)N(Cc1ncc(-c2nnc(C(F)F)o2)s1)c1cnn(C(F)F)c1] | 1H NMR (400 MHz, CDCl3) δ ppm 8.35 (s, 1 H), 7.92 (s, 1 H), 7.66 (s, 1 H), 6.96 (t, J = 60.4 Hz, 1 H), 6.84 (t, J = 45.2 Hz, 1 H), 5.11 (s, 2 H), 3.10 (q, J = 7.2 Hz, 2 H), 1.33 (t, J = 7.6 Hz, 3 H). LCMS RT = 3.543 min, m/z = 441.1 | 0.262 |
| I-49 | 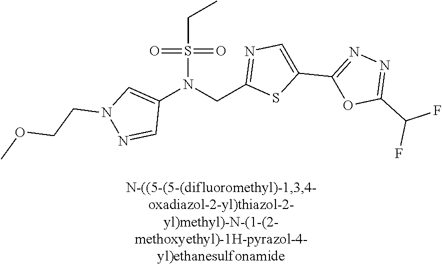 [SMILES: CCS(=O)(=O)N(Cc1ncc(-c2nnc(C(F)F)o2)s1)c1cnn(CCOC)c1] | 1H NMR (400 MHz, CDCl3) δ ppm 8.40 (s, 1 H), 7.65 (s, 1 H), 7.51 (s, 1 H), 6.91 (t, J = 51.6 Hz, 1 H), 5.15 (s, 2 H), 4.22 (t, J = 4.8 Hz, 2 H), 3.70 (t, J = 5.2 Hz, 2 H), 3.30 (s, 3 H), 3.17 (q, J = 7.6 Hz, 2 H), 1.41 (t, J = 7.2 Hz, 3 H). LCMS RT = 3.093 min, m/z = 448.9 | 0.134 |
| I-50 | 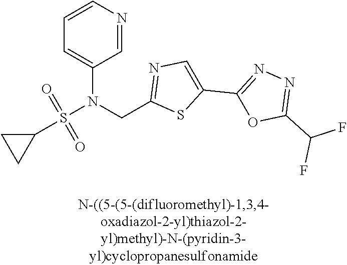 [SMILES: O=S(=O)(C1CC1)N(Cc1ncc(-c2nnc(C(F)F)o2)s1)c1cccnc1] | 1H NMR (400 MHz, CDCl3) δ ppm 8.69 (d, J = 2.0 Hz, 1 H), 8.53-8.50 (m, 1 H), 8.30 (s, 1 H), 7.79-7.76 (m, 1 H), 7.30- 7.26 (m, 1 H), 6.83 (t, J = 51.6 Hz, 1 H), 5.23 (s, 2 H), 2.53- 2.46 (m, 1 H), 1.06-0.95 (m, 4 H). LCMS RT = 2.897 min, m/z = 414.1 | 0.036 |
| I-51 | 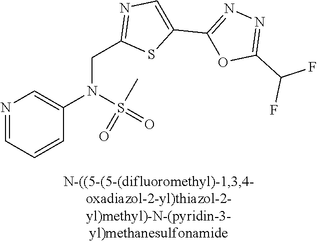 [SMILES: CS(=O)(=O)N(Cc1ncc(-c2nnc(C(F)F)o2)s1)c1cccnc1] | 1H NMR (400 MHz, CDCl3) δ ppm 8.54 (s, 1 H), 8.50 (s, 1 H), 7.93-7.90 (m, 1 H), 7.68 (d, J = 8.0 Hz, 1 H), 7.49-7.44 (m, 1 H), 6.92 (t, J = 51.6 Hz, 1 H), 5.84 (s, 2 H), 3.00 (s, 3 H). LCMS RT = 1.096 min, m/z = 387.9 | 0.676 |
| I-52 | 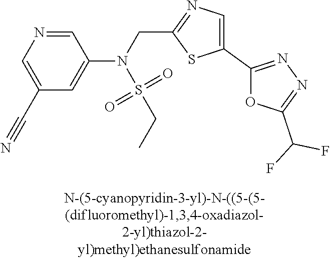 [SMILES: CCS(=O)(=O)N(Cc1ncc(-c2nnc(C(F)F)o2)s1)c1cncc(C#N)c1] | 1H NMR (400 MHz, CDCl3) δ ppm 8.96 (d, J = 2.8 Hz, 1 H), 8.82 (s, 1 H), 8.42 (s, 1 H), 8.18- 8.17 (m, 1 H), 6.92 (t, J = 51.6 Hz, 1 H), 5.30 (s, 2 H), 3.21 (q, J = 7.6 Hz, 2 H), 1.44 (t, J = 7.6 Hz, 3 H). LCMS RT = 0.752 min, m/z = 427.1 | 0.022 |
| I-53 | 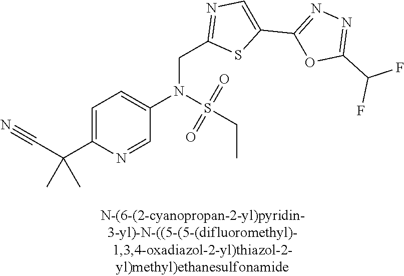 [SMILES: CCS(=O)(=O)N(Cc1ncc(-c2nnc(C(F)F)o2)s1)c1ccc(C(C)(C)C#N)nc1] | 1H NMR (400 MHz, CDCl3) δ ppm 8.70 (d, J = 2.8 Hz, 1 H), 8.40 (s, 1 H), 7.86 (d, J = 8.4 Hz, 1 H), 7.63 (d, J = 8.4 Hz, 1 H), 6.91 (t, J = 51.6 Hz, 1 H), 5.29 (s, 2 H), 3.22 (q, J = 7.6 Hz, 2 H), 1.75 (s, 6 H), 1.47 (t, J = 7.2 Hz, 3 H). LCMS RT = 0.828 min, m/z = 469.2 | 0.317 |
| I-54 | 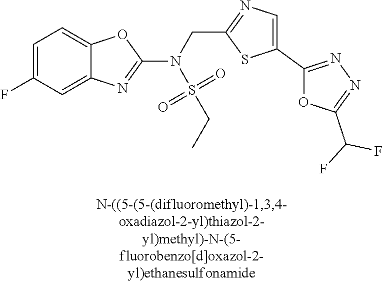 [SMILES: CCS(=O)(=O)N(Cc1ncc(-c2nnc(C(F)F)o2)s1)c1nc2cc(F)ccc2o1] | 1H NMR (400 MHz, CDCl3) δ ppm 8.45 (s, 1 H), 7.40-7.38 (m, 1 H), 7.10-6.91 (m, 3 H), 5.40 (s, 2 H), 3.29 (q, J = 7.2 Hz, 2 H), 1.52 (t, J = 7.2 Hz, 3 H). LCMS RT = 3.800 min, m/z = 460.0 | 0.104 |
| I-55 | 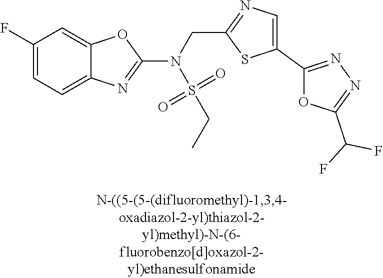 [SMILES: CCS(=O)(=O)N(Cc1ncc(-c2nnc(C(F)F)o2)s1)c1nc2ccc(F)cc2o1] | 1H NMR (400 MHz, CDCl3) δ ppm 8.44 (s, 1 H), 7.29-7.21 (m, 2 H), 7.10-7.04 (m, 1 H), 6.91 (t, J = 51.6 Hz, 1 H), 5.42 (s, 2 H), 3.29 (q, J = 7.2 Hz, 2 H), 1.52 (t, J = 7.2 Hz, 3 H). LCMS RT = 0.863 min, m/z = 459.7 | 0.124 |
| I-56 | 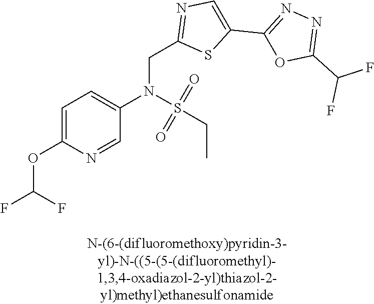 [SMILES: CCS(=O)(=O)N(Cc1ncc(-c2nnc(C(F)F)o2)s1)c1ccc(OC(F)F)nc1] | 1H NMR (400 MHz, CDCl3) δ ppm 8.39 (s, 1 H), 8.28 (d, J = 2.8 Hz, 1 H), 7.85 (d, J = 8.8 Hz, 1 H), 7.41 (t, J = 51.6 Hz, 1 H), 7.06-6.76 (m, 2 H), 5.24 (s, 2 H), 3.20 (q, J = 7.2 Hz, 2 H), 1.47 (t, J = 7.6 Hz, 3 H). LCMS RT = 0.886 min, m/z = 468.1 | 0.050 |
| I-57 | 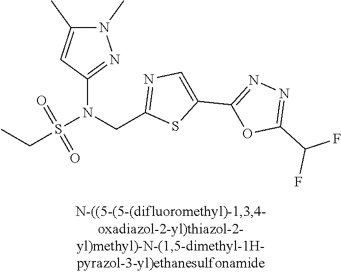 [SMILES: CCS(=O)(=O)N(Cc1ncc(-c2nnc(C(F)F)o2)s1)c1cc(C)n(C)n1] | 1H NMR (400 MHz, CDCl3) δ ppm 8.40 (s, 1 H), 6.91 (t, J = 51.6 Hz, 1 H), 6.13 (s, 1 H), 5.37 (s, 2 H), 3.70 (s, 3 H), 3.27 (q, J = 7.6 Hz, 2 H), 2.24 (s, 3 H), 1.42 (t, J = 7.2 Hz, 3 H). LCMS RT = 3.486 min, m/z = 418.9 | 0.117 |
| I-58 | 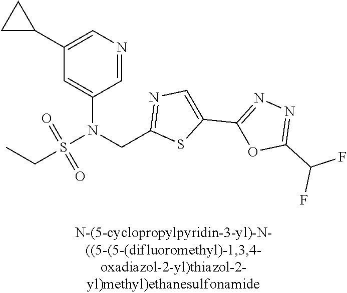 [SMILES: CCS(=O)(=O)N(Cc1ncc(-c2nnc(C(F)F)o2)s1)c1cncc(C2CC2)c1] | 1H NMR (400 MHz, CDCl3) ppm 8.45 (s, 1 H), 8.41 (s, 1 H), 8.35 (s, 1 H), 7.46 (s, 1 H), 6.91 (t, J = 51.6 Hz, 1 H), 5.27 (s, 2 H), 3.19 (q, J = 8.0 Hz, 2 H), 1.92- 1.89 (m, 1 H), 1.45 (t, J = 7.2 Hz, 3 H), 1.11-1.05 (m, 2 H), 0.76-0.74 (m, 2 H). LCMS RT = 0.992 min, m/z = 442.3 | 0.025 |
| I-59 | 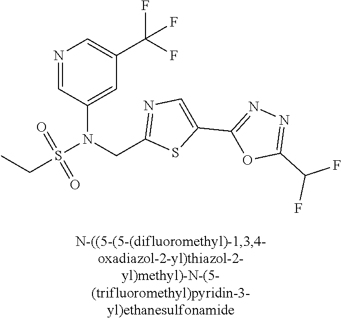 [SMILES: CCS(=O)(=O)N(Cc1ncc(-c2nnc(C(F)F)o2)s1)c1cncc(C(F)(F)F)c1] | 1H NMR (400 MHz, CDCl3) δ ppm 8.93 (d, J = 2.0 Hz, 1 H), 8.83 (s, 1 H), 8.41 (s, 1 H), 8.10 (s, 1 H), 6.91 (t, J = 51.6 Hz, 1 H), 5.31 (s, 2 H), 3.22 (q, J = 7.6 Hz, 2 H), 1.45 (t, J = 7.6 Hz, 3 H). LCMS RT = 1.190 min, m/z = 469.8 | 0.030 |
| I-60 | 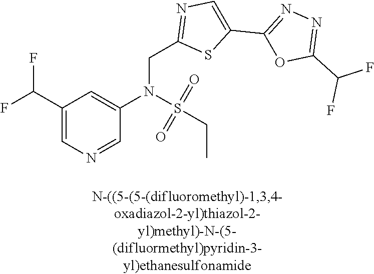 [SMILES: CCS(=O)(=O)N(Cc1ncc(-c2nnc(C(F)F)o2)s1)c1cncc(C(F)F)c1] | 1H NMR (400 MHz, CDCl3) δ ppm 8.85 (s, 1 H), 8.73 (s, 1 H), 8.40 (s, 1 H), 7.99 (s, 1 H), 6.91 (t, J = 52.0 Hz, 1 H), 6.74 (t, J = 56.0 Hz, 1 H), 5.31 (s, 2 H), 3.22 (q, J = 4.0 Hz, 2 H), 1.46 (t, J = 8.0 Hz, 3 H). LCMS RT = 2.508 min, m/z = 452.1 | 0.070 |
| I-61 | 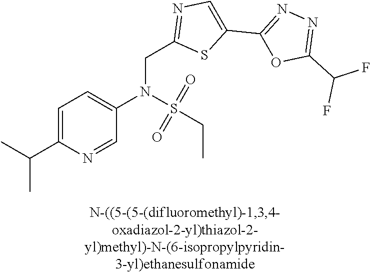 [SMILES: CCS(=O)(=O)N(Cc1ncc(-c2nnc(C(F)F)o2)s1)c1ccc(C(C)C)nc1] | 1H NMR (400 MHz, CDCl3) δ ppm 8.59 (d, J = 2.4 Hz, 1 H), 8.38 (s, 1 H), 7.75-7.71 (m, 1 H), 7.22 (d, J = 8.0 Hz, 1H), 6.91 (t, J = 51.6 Hz, 1 H), 5.27 (s, 2 H), 3.20 (q, J = 7.2 Hz, 2 H), 3.11- 3.03 (m, 1 H), 1.47 (t, J = 7.2 Hz, 3 H), 1.29 (d, J = 7.2 Hz, 6 H). LCMS RT = 0.757 min, m/z = 444.2 | 0.073 |
| I-62 | 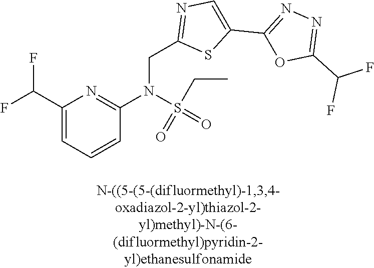 [SMILES: CCS(=O)(=O)N(Cc1ncc(-c2nnc(C(F)F)o2)s1)c1cccc(C(F)F)n1] | 1H NMR (400 MHz, CDCl3) δ ppm 8.30 (s, 1 H), 7.85-7.69 (m, 2 H), 7.39 (d, J = 7.6 Hz, 1 H), 6.81 (t, J = 51.6 Hz, 1 H), 6.47 (t, J = 79.2 Hz, 1 H), 5.54 (s, 2 H), 3.29 (q, J = 7.6 Hz, 2 H), 1.33 (t, J = 7.6 Hz, 3 H). LCMS RT = 2.238 min, m/z = 452.1 | 0.0206 |
| I-63 | 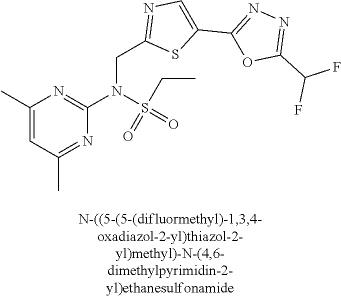 [SMILES: CCS(=O)(=O)N(Cc1ncc(-c2nnc(C(F)F)o2)s1)c1nc(C)cc(C)n1] | 1H NMR (400 MHz, CDCl3) δ ppm 8.40 (s, 1 H), 6.89 (t, J = 51.6 Hz, 1 H), 6.77 (s, 1 H), 5.65 (s, 2 H), 3.95 (q, J = 7.6 Hz, 2 H), 2.44 (s, 6 H), 1.39 (t, J = 7.6 Hz, 3 H). LCMS RT = 3.758 min, m/z = 431.2 | 0.018 |
| I-64 | 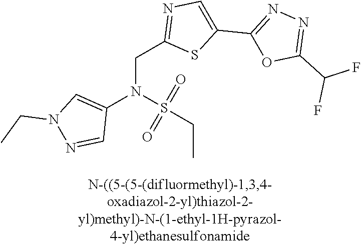 [SMILES: CCn1cc(N(Cc2ncc(-c3nnc(C(F)F)o3)s2)S(=O)(=O)CC)cn1] | 1H NMR (400 MHz, CDCl3) δ ppm 8.40 (s, 1 H), 7.60 (s, 1 H), 7.49 (s, 1 H), 6.91 (t, J = 51.6 Hz, 1 H), 5.16 (s, 2 H), 4.12 (q, J = 7.2 Hz, 2 H), 3.17 (q, J = 7.6 Hz, 2 H), 1.50-1.40 (m, 6 H). LCMS RT = 1.418 min, m/z = 418.9 | 0.065 |
| I-65 | 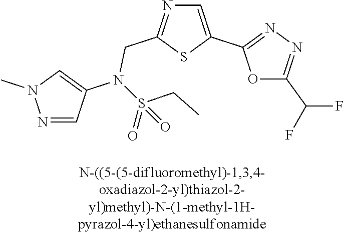 [SMILES: CCS(=O)(=O)N(Cc1ncc(-c2nnc(C(F)F)o2)s1)c1cnn(C)c1] | 1H NMR (400 MHz, CD3OD) 8 ppm 8.33 (s, 1 H), 7.71 (s, 1 H), 7.48 (s, 1 H), 7.11 (t, J = 51.6 Hz, 1 H), 5.10 (s, 2 H), 3.74 (s, 3 H), 3.14 (q, J = 7.2 Hz, 2 H), 1.28 (t, J = 7.2 Hz, 3 H). LCMS RT = 2.950 min, m/z = 404.9 | 0.022 |
| I-66 | 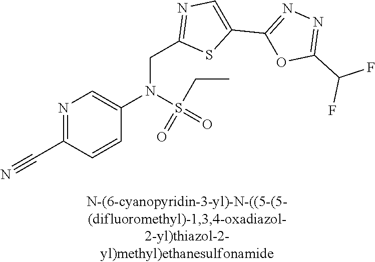 [SMILES: CCS(=O)(=O)N(Cc1ncc(-c2nnc(C(F)F)o2)s1)c1ccc(C#N)nc1] | 1H NMR (400 MHz, CDCl3) δ ppm 8.87 (s, 1 H), 8.40 (s, 1 H), 8.04 (d, J = 8.4 Hz, 1 H), 7.74 (d, J = 8.4 Hz, 1 H), 6.91 (t, J = 51.6 Hz, 1 H), 5.35 (s, 2 H), 3.23 (q, J = 7.2 Hz, 2 H), 1.43 (t, J = 7.2 Hz, 3 H). LCMS RT = 1.030 min, m/z = 426.8 | 0.029 |
| I-67 | 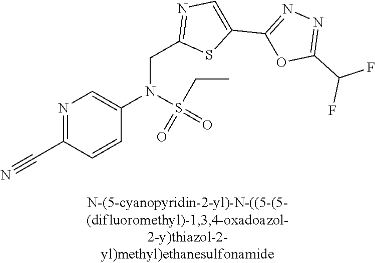 [SMILES: CCS(=O)(=O)N(Cc1ncc(-c2nnc(C(F)F)o2)s1)c1ccc(C#N)nc1] | 1H NMR (400 MHz, CDCl3) δ ppm 8.66 (s, 1 H), 8.39 (s, 1 H), 7.96-7.78 (m, 2 H), 6.90 (t, J = 51.6 Hz, 1 H), 5.63 (s, 2 H), 3.48 (q, J = 7.6 Hz, 2 H), 1.43 (t, J = 7.6 Hz, 3 H). LCMS RT = 2.180 min, m/z = 427.1 | 0.037 |
| I-68 | 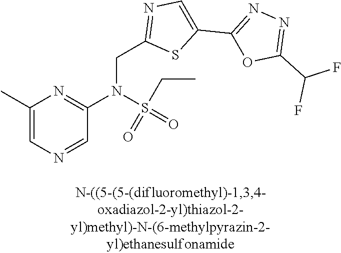 [SMILES: CCS(=O)(=O)N(Cc1ncc(-c2nnc(C(F)F)o2)s1)c1cncc(C)n1] | 1H NMR (400 MHz, CDCl3) δ ppm 8.80 (s, 1 H), 8.39 (s, 1 H), 8.29 (s, 1 H), 6.90 (t, J = 51.6 Hz, 1 H), 5.58 (s, 2 H), 3.38 (q, J = 7.2 Hz, 2 H), 2.55 (s, 3 H), 1.42 (t, J = 7.6 Hz, 3 H). LCMS RT = 1.657 min, m/z = 417.1 | 0.100 |
| I-69 | 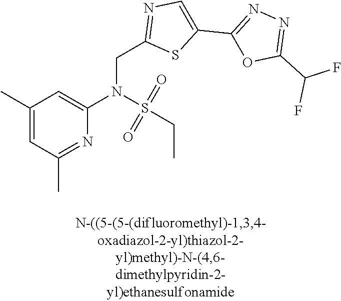 [SMILES: CCS(=O)(=O)N(Cc1ncc(-c2nnc(C(F)F)o2)s1)c1cc(C)cc(C)n1] | 1H NMR (400 MHz, CDCl3) δ ppm 8.38 (s, 1 H), 7.27 (s, 1 H), 7.02-6.72 (m, 2 H), 5.58 (s, 2 H), 3.24 (q, J = 7.6 Hz, 2 H), 2.47 (s, 3 H), 2.33 (s, 3 H), 1.36 (t, J = 7.4 Hz, 3 H). LCMS RT = 3.082 min, m/z = 429.9 | 0.071 |
| I-70 | 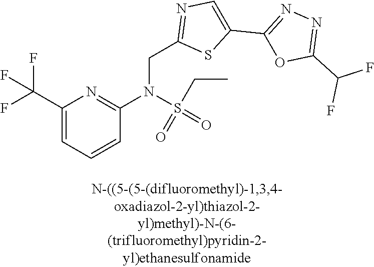 [SMILES: CCS(=O)(=O)N(Cc1ncc(-c2nnc(C(F)F)o2)s1)c1cccc(C(F)(F)F)n1] | 1H NMR (400 MHz, CD3OD) δ ppm 8.42 (s, 1 H), 8.07-8.02 (m, 1 H), 7.92-7.89 (m, 1 H), 7.61- 7.58 (m, 1 H), 7.19 (t, J = 51.6 Hz, 1 H), 5.62 (s, 2 H), 3.57 (q, J = 7.2 Hz, 2 H), 1.38 (t, J = 7.2 Hz, 3 H). LCMS RT = 2.965 min, m/z = 470.1 | 0.0281 |
| I-71 | 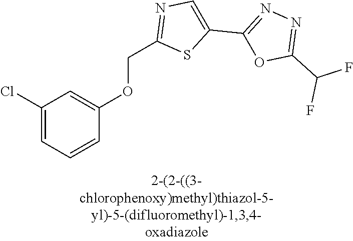 [SMILES: FC(F)c1nnc(-c2cnc(COc3cccc(Cl)c3)s2)o1] | 1H NMR (400 MHz, CDCl3) δ ppm 8.49 (s, 1 H), 7.29-7.24 (m, 1 H), 7.06-7.04 (m, 2 H), 6.94- 6.78 (m, 2 H), 5.43 (s, 2 H). LCMS RT = 1.840 min, m/z = 344.1 | 0.251 |
| I-72 | 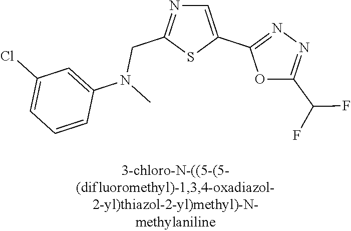 [SMILES: CN(Cc1ncc(-c2nnc(C(F)F)o2)s1)c1cccc(Cl)c1] | 1H NMR (400 MHz, CDCl3) δ ppm 8.55 (s, 1 H), 7.43-6.70 (m, 5 H), 4.93 (s, 2 H), 3.26 (s, 3 H). LCMS RT = 3.547 min, m/z = 356.8 | 0.034 |
| I-73 | 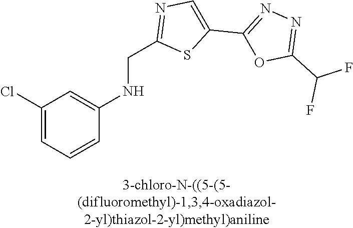 [SMILES: FC(F)c1nnc(-c2cnc(CNc3cccc(Cl)c3)s2)o1] | 1H NMR (400 MHz, CDCl3) δ ppm 8.37 (s, 1 H), 7.06-6.44 (m, 5 H), 4.66 (s, 3 H). LCMS RT = 2.368 min, m/z = 343.1 | 0.046 |
| I-74 | 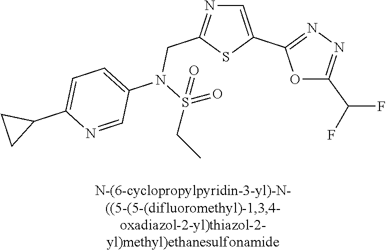 [SMILES: CCS(=O)(=O)N(Cc1ncc(-c2nnc(C(F)F)o2)s1)c1ccc(C2CC2)nc1] | 1H NMR (400 MHz, CD3OD) δ ppm 8.46 (d, J = 2.4 Hz, 1 H), 8.41 (s, 1 H), 7.80 (dd, J = 8.4, 2.8 Hz, 1 H), 7.33-7.07 (m, 2 H), 5.33 (s, 2 H), 3.31-3.27 (m, 2 H), 2.11-2.08 (m, 1 H), 1.40 (t, J = 7.2 Hz, 3 H), 1.04-0.94 (m, 4 H). LCMS RT = 5.273 min, m/z = 441.9 | 0.016 |
| I-75 | 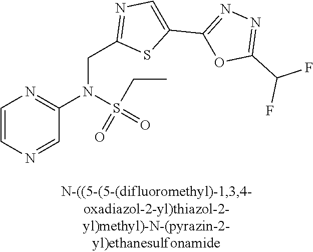 [SMILES: CCS(=O)(=O)N(Cc1ncc(-c2nnc(C(F)F)o2)s1)c1cnccn1] | 1H NMR (400 MHz, CDCl3) δ ppm 9.00 (s, 1 H), 8.43 (d, J = 2.8 Hz, 1 H), 8.39-8.35 (m, 2 H), 6.89 (t, J = 51.6 Hz, 1 H), 5.58 (s, 2 H), 3.41 (q, J = 7.6 Hz, 2 H), 1.43 (t, J = 7.2 Hz, 3 H). LCMS RT = 2.823 min, m/z = 403.1 | 0.023 |
| I-76 | 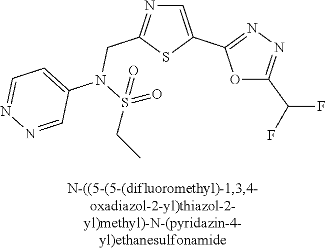 [SMILES: CCS(=O)(=O)N(Cc1ncc(-c2nnc(C(F)F)o2)s1)c1ccnnc1] | 1H NMR (400 MHz, CD3OD) δ ppm 8.57 (d, J = 7.6 Hz, 1 H), 8.53 (s, 1 H), 8.23 (s, 1 H), 7.42- 7.40 (m, 1 H), 7.22 (t, J = 51.6 Hz, 1 H), 5.85 (s, 2 H), 3.10 (q, J = 7.6 Hz, 2 H), 1.36 (t, J = 7.2 Hz, 3 H). LCMS RT = 3.671 min, m/z = 402.9 | 0.097 |
| I-77 | 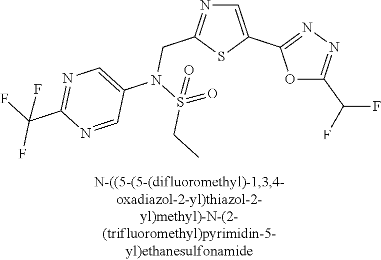 [SMILES: CCS(=O)(=O)N(Cc1ncc(-c2nnc(C(F)F)o2)s1)c1cnc(C(F)(F)F)nc1] | 1H NMR (400 MHz, CDCl3) δ ppm 9.10 (s, 2 H), 8.43 (s, 1 H), 6.92 (t, J = 51.6 Hz, 1 H), 5.32 (d, J = 12 Hz, 2 H), 3.25 (q, J = 7.2 Hz, 2 H), 1.44 (t, J = 7.2 Hz, 3 H). LCMS RT = 2.594 min, m/z = 471.1 | 0.155 |
| I-78 | 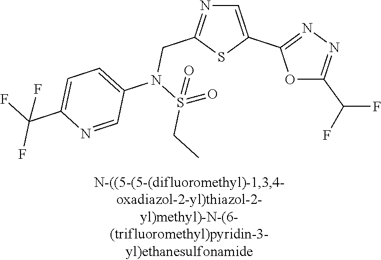 [SMILES: CCS(=O)(=O)N(Cc1ncc(-c2nnc(C(F)F)o2)s1)c1ccc(C(F)(F)F)nc1] | 1H NMR (400 MHz, CDCl3) δ ppm 8.86 (s, 1 H), 8.40 (s, 1 H), 8.10 (d, J = 8.4 Hz, 1 H), 7.74 (d, J = 8.4 Hz, 1 H), 6.91 (t, J = 51.6 Hz, 1 H), 5.34 (s, 2 H), 3.23 (q, J = 7.2 Hz, 2 H), 1.45 (t, J = 7.2 Hz, 3 H). LCMS RT = 0.882 min, m/z = 470.1 | 0.059 |
| I-79 | 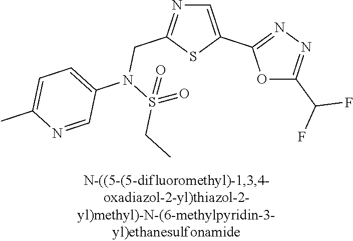 [SMILES: CCS(=O)(=O)N(Cc1ncc(-c2nnc(C(F)F)o2)s1)c1ccc(C)nc1] | 1H NMR (400 MHz, CDCl3) δ ppm 8.55 (s, 1 H), 8.37 (s, 1 H), 7.73-7.68 (m, 1 H), 7.20 (d, J = 7.6 Hz, 1 H), 6.91 (t, J = 51.6 Hz, 1 H), 5.27 (s, 2 H), 3.20 (q, J = 7.2 Hz, 2 H), 2.56 (s, 3 H), 1.46 (t, J = 7.2 Hz, 3 H). LCMS RT = 0.616 min, m/z = 416.2 | 0.073 |
| I-80 | 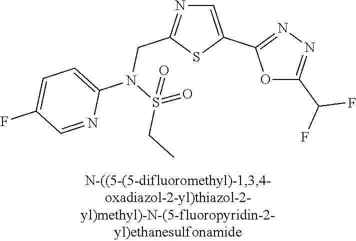 [SMILES: CCS(=O)(=O)N(Cc1ncc(-c2nnc(C(F)F)o2)s1)c1ccc(F)cn1] | 1H NMR (400 MHz, CDCl3) δ ppm 8.37 (s, 1 H), 8.28 (s, 1 H), 7.70-7.60 (m, 1 H), 7.50-7.40 (m, 1 H), 6.89 (t, J = 51.6 Hz, 1 H), 5.54 (s, 2 H), 3.25 (q, J = 7.2 Hz, 2 H), 1.40 (t, J = 7.6 Hz, 3 H). LCMS RT = 2.661 min, m/z = 419.9 | 0.0265 |
| I-81 | 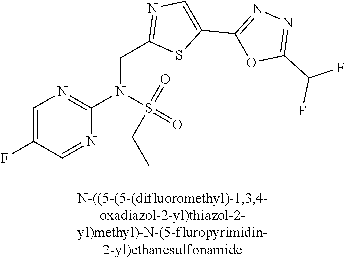 [SMILES: CCS(=O)(=O)N(Cc1ncc(-c2nnc(C(F)F)o2)s1)c1ncc(F)cn1] | 1H NMR (400 MHz, CDCl3) δ ppm 8.49 (s, 2 H), 8.40 (s, 1 H), 6.90 (t, J = 51.6 Hz, 1 H), 5.65 (s, 2 H), 3.91 (q, J = 7.6 Hz, 2 H), 1.45 (t, J = 7.6 Hz, 3 H). LCMS RT = 1.977 min, m/z = 421.1 | 0.116 |
| I-82 | 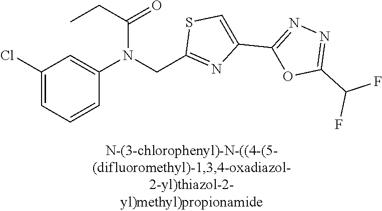 [SMILES: CCC(=O)N(Cc1nc(-c2nnc(C(F)F)o2)cs1)c1cccc(Cl)c1] | 1H NMR (400 MHz, CDCl3) δ ppm 8.28 (s, 1 H), 7.37-7.32 (m, 2 H), 7.25-7.23 (m, 1 H), 7.10- 7.07 (m, 1 H), 6.91 (t, J = 51.6 Hz, 1 H), 5.22 (s, 2 H), 2.19 (q, J = 7.2 Hz, 2 H), 1.21 (t, J = 7.2 Hz, 3 H). LCMS RT = 0.931 min, m/z = 399.1 | 4.1 |
| I-83 | 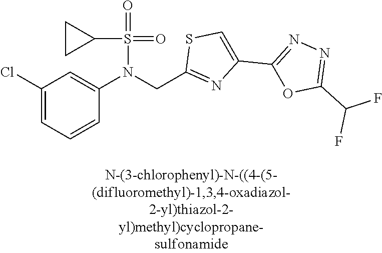 [SMILES: O=S(=O)(C1CC1)N(Cc1nc(-c2nnc(C(F)F)o2)cs1)c1cccc(Cl)c1] | 1H NMR (400 MHz, CDCl3) δ ppm 8.26 (s, 1 H), 7.51 (s, 1 H), 7.41-7.30 (m, 3 H), 6.91 (t, J = 51.6 Hz, 1 H), 5.32 (s, 2 H), 2.59- 2.52 (m, 1 H), 1.18-1.13 (m, 2 H), 1.08-1.04 (m, 2 H). LCMS RT = 0.944 min, m/z = 447.1 | 0.759 |
| I-84 | 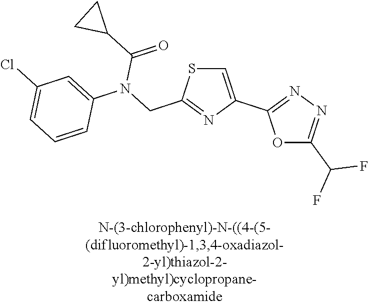 [SMILES: O=C(C1CC1)N(Cc1nc(-c2nnc(C(F)F)o2)cs1)c1cccc(Cl)c1] | 1H NMR (400 MHz, CDCl3) δ ppm 8.27 (s, 1 H), 7.40-7.20 (m, 4 H), 6.91 (t, J = 51.6 Hz, 1 H), 5.25 (s, 2 H), 1.50-1.40 (m, 1 H), 1.18-1.09 (m, 2 H), 0.83- 0.74 (m, 2 H). LCMS RT = 1.163 min, m/z = 410.8 | 7.5 |
| I-85 | 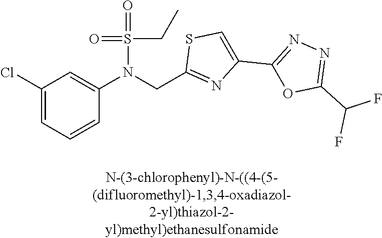 [SMILES: CCS(=O)(=O)N(Cc1nc(-c2nnc(C(F)F)o2)cs1)c1cccc(Cl)c1] | 1H NMR (400 MHz, CDCl3) δ ppm 8.26 (s, 1 H), 7.48 (s, 1 H), 7.37-7.31 (m, 3 H), 6.91 (t, J = 51.6 Hz, 1 H), 5.32 (s, 2 H), 3.19 (q, J = 7.2 Hz, 2 H), 1.46 (t, J = 7.2 Hz, 3 H). LCMS RT = 0.922 min, m/z = 435.0 | 0.584 |
| I-86 | 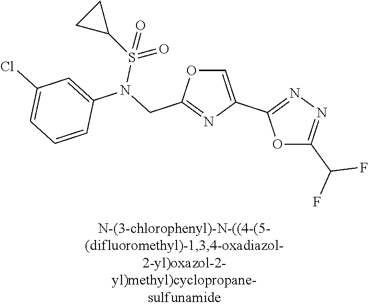 [SMILES: O=S(=O)(C1CC1)N(Cc1nc(-c2nnc(C(F)F)o2)co1)c1cccc(Cl)c1] | 1H NMR (400 MHz, CDCl3) δ ppm 8.36 (s, 1 H), 7.50 (s, 1 H), 7.39-7.37 (m, 1 H), 7.33-7.30 (m, 2 H), 6.91 (t, J = 51.6 Hz, 1 H), 5.03 (s, 2 H), 2.69-2.65 (m, 1 H), 1.09-0.90 (m, 4 H). LCMS RT = 3.210 min, m/z = 431.1 | 8.0 |
| I-87 | 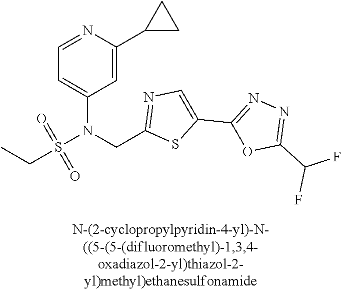 [SMILES: CCS(=O)(=O)N(Cc1ncc(-c2nnc(C(F)F)o2)s1)c1ccnc(C2CC2)c1] | 1H NMR (400 MHz, CDCl3) δ 8.42-8.36 (m, 2 H), 7.31 (s, 1 H), 7.18-7.14 (m, 1 H), 6.90 (t, J = 51.6 Hz, 1 H), 5.37 (s, 2 H), 3.26 (q, J = 7.6 Hz, 2 H), 2.04- 1.95 (m, 1 H), 1.42 (t, J = 7.2 Hz, 3 H), 1.05-1.00 (m, 4 H). LCMS RT = 1.094 min, m/z = 441.9 | 0.039 |
| I-88 | 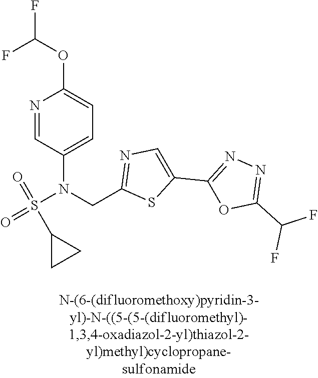 [SMILES: O=S(=O)(C1CC1)N(Cc1ncc(-c2nnc(C(F)F)o2)s1)c1ccc(OC(F)F)nc1] | 1H NMR (400 MHz, CDCl3) δ 8.39 (s, 1 H), 8.33 (d, J = 2.8 Hz, 1 H), 7.87 (dd, J = 2.8, 8.8 Hz, 1 H), 7.42 (t, J = 72.0 Hz, 1 H), 7.06-6.77 (m, 2 H), 5.26 (s, 2 H), 2.64-2.48 (m, 1 H), 1.21- 0.98 (m, 4 H). LCMS RT = 0.957 min, m/z = 479.9 | 0.048 |
| I-89 | 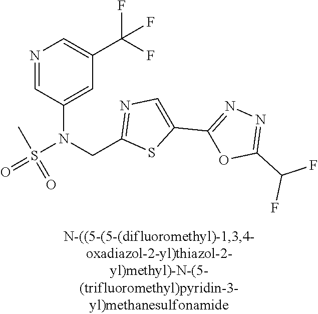 [SMILES: CS(=O)(=O)N(Cc1ncc(-c2nnc(C(F)F)o2)s1)c1cncc(C(F)(F)F)c1] | 1H NMR (400 MHz, CDCl3) δ 8.95 (d, J = 2.4 Hz, 1 H), 8.87 (d, J = 2.4 Hz, 1 H), 8.44 (s, 1 H), 8.13 (s, 1 H), 6.92 (t, J = 51.6 Hz, 1 H), 5.31 (s, 2 H), 3.12 (s, 3 H). LCMS RT = 0.878 min, m/z = 455.9 | 0.049 |
| I-90 | 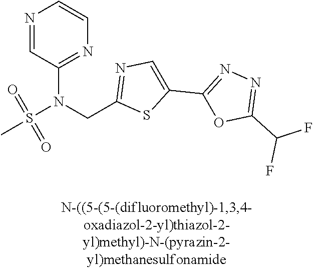 [SMILES: CS(=O)(=O)N(Cc1ncc(-c2nnc(C(F)F)o2)s1)c1cnccn1] | 1H NMR (400 MHz, CDCl3) δ 9.00 (s, 1 H), 8.47-8.36 (m, 3 H), 6.91 (t, J = 51.2 Hz, 1 H), 5.58 (s, 2 H), 3.27 (s, 3 H). LCMS RT = 0.943 min, m/z = 389.1 | 0.041 |
| I-91 | δ 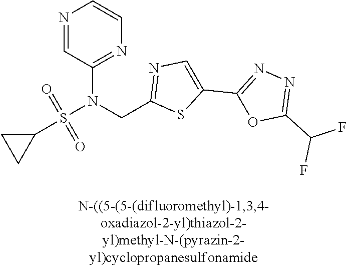 [SMILES: O=S(=O)(C1CC1)N(Cc1ncc(-c2nnc(C(F)F)o2)s1)c1cnccn1] | 1H NMR (400 MHz, CDCl3) δ 9.02 (d, J = 1.2 Hz, 1 H), 8.46- 8.35 (m, 3 H), 6.91 (t, J = 51.6 Hz, 1 H), 5.58 (s, 2 H), 2.72- 2.61 (m, 1 H), 1.27-1.21 (m, 2 H), 1.11-1.01 (m, 2 H). LCMS RT = 1.031 min, m/z = 415.1 | 0.018 |
| I-92 | 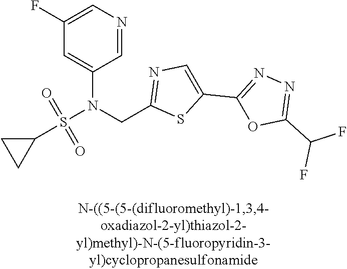 [SMILES: O=S(=O)(C1CC1)N(Cc1ncc(-c2nnc(C(F)F)o2)s1)c1cncc(F)c1] | 1H NMR (400 MHz, CDCl3) δ 8.61 (s, 1 H), 8.47 (d, J = 4.0 Hz, 1 H), 8.40 (s, 1 H), 7.68-7.62 (m, 1 H), 6.91 (t, J = 51.6 Hz, 1 H), 5.31 (s, 2 H), 2.63-2.52 (m, 1 H), 1.17-1.02 (m, 4 H). LCMS RT = 2.295 min, m/z = 431.9 | 0.014 |
| I-93 | 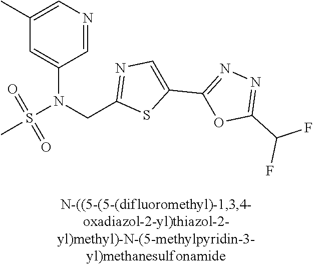 [SMILES: Cc1cncc(N(Cc2ncc(-c3nnc(C(F)F)o3)s2)S(C)(=O)=O)c1] | 1H NMR (400 MHz, CDCl3) δ 8.52-8.43 (m, 2 H), 8.40 (s, 1 H), 7.65 (s, 1 H), 6.91 (t, J = 51.6 Hz, 1 H), 5.27 (s, 2 H), 3.10 (s, 3 H), 2.39 (s, 3 H). LCMS RT = 0.828 min, m/z = 401.9 | 0.055 |
| I-94 | 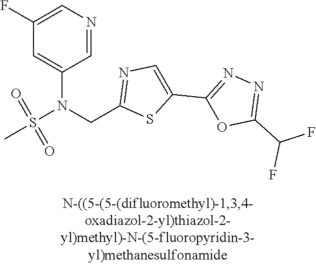 [SMILES: CS(=O)(=O)N(Cc1ncc(-c2nnc(C(F)F)o2)s1)c1cncc(F)c1] | 1H NMR (400 MHz, CDCl3) δ 8.58 (s, 1 H), 8.49 (s, 1 H), 8.43 (s, 1 H), 7.68-7.65 (m, 1 H), 6.92 (t, J = 51.6 Hz, 1 H), 5.29 (s, 2 H), 3.12 (s, 3 H). LCMS RT = 1.877 min, m/z = 406.1 | 0.025 |
| I-95 | 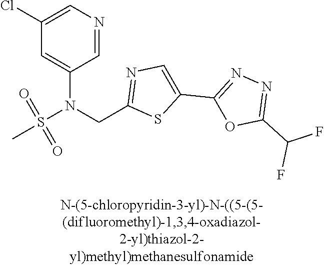 [SMILES: CS(=O)(=O)N(Cc1ncc(-c2nnc(C(F)F)o2)s1)c1cncc(Cl)c1] | 1H NMR (400 MHz, CDCl3) δ 8.62 (d, J = 2.4 Hz, 1 H), 8.57 (d, J = 2.4 Hz, 1 H), 8.43 (s, 1 H), 7.90 (s, 1 H), 6.92 (t, J = 51.6 Hz, 1 H), 5.27 (s, 2 H), 3.11 (s, 3 H). LCMS RT = 2.188 min, m/z = 421.8 | 0.0235 |
| I-96 | 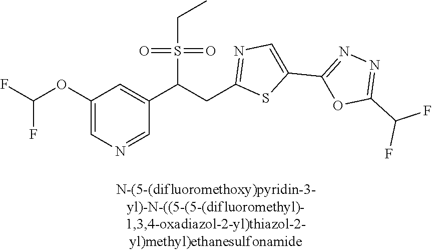 [SMILES: CCS(=O)(=O)C(Cc1ncc(-c2nnc(C(F)F)o2)s1)c1cncc(OC(F)F)c1] | 1H NMR (400 MHz, CDCl3) δ 8.63 (s, 1 H), 8.46 (s, 1 H), 8.39 (s, 1 H), 7.68 (s, 1 H), 6.91 (t, J = 51.6 Hz, 1 H), 6.58 (t, J = 51.6 Hz, 1 H), 5.29 (s, 2 H), 3.22 (q, J = 7.2 Hz , 2 H), 1.44 (t, J = 7.6 Hz, 3 H). LCMS RT = 0.907 min, m/z = 467.8 | 0.010 |
| I-97 | 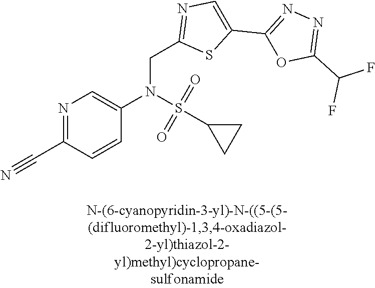 [SMILES: N#Cc1ccc(N(Cc2ncc(-c3nnc(C(F)F)o3)s2)S(=O)(=O)C2CC2)cn1] | 1H NMR (400 MHz, CDCl3) δ 8.89 (d, J = 2.4 Hz, 1 H), 8.39 (s, 1 H), 8.05 (d, J = 8.4 Hz, 1 H), 7.74 (d, J = 8.8 Hz, 1 H), 6.91 (t, J = 51.6 Hz, 1 H), 5.35 (s, 2 H), 2.59 - 2.53 (m, 1 H), 1.16-1.07 (m, 4 H). LCMS RT = 0.859 min, m/z = 439.0 | 0.010 |
| I-98 | 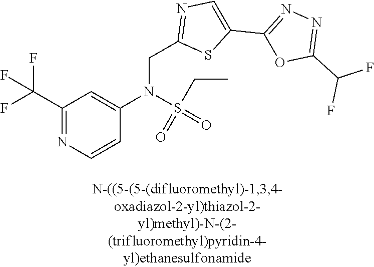 [SMILES: CCS(=O)(=O)N(Cc1ncc(-c2nnc(C(F)F)o2)s1)c1ccnc(C(F)(F)F)c1] | 1H NMR (400 MHz, CDCl3) δ 8.59 (d, J = 5.6 Hz, 1 H), 8.39 (s, 1 H), 7.81 (s, 1 H), 7.61-7.53 (m, 1 H), 6.83 (t, J = 51.6 Hz, 1 H), 5.35 (s, 2 H), 3.24 (q, J = 7.2 Hz, 2 H), 1.35 (t, J = 7.2 Hz, 3 H). LCMS RT = 0.956 min, m/z = 469.9 | 0.069 |
| I-99 | 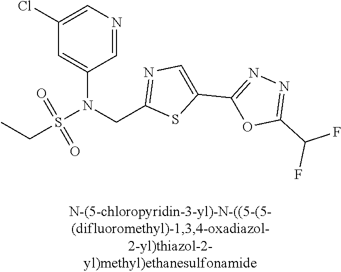 [SMILES: CCS(=O)(=O)N(Cc1ncc(-c2nnc(C(F)F)o2)s1)c1cncc(Cl)c1] | 1H NMR (400 MHz, CDCl3) δ 8.61 (d, J = 2.0 Hz, 1 H), 8.55 (d, J = 2.0 Hz, 1 H), 8.41 (s, 1 H), 7.89 (t, J = 2.2 Hz, 1 H), 6.92 (t, J = 51.6 Hz, 1 H), 5.28 (s, 2 H), 3.22 (q, J = 7.2 Hz, 2 H), 1.46 (t, J = 7.6 Hz, 3 H). LCMS RT = 2.461 min, m/z = 435.9 | 0.0189 |
| I-100 | 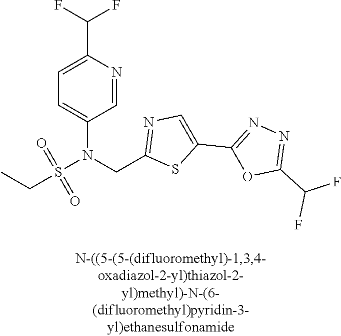 [SMILES: CCS(=O)(=O)N(Cc1ncc(-c2nnc(C(F)F)o2)s1)c1ccc(C(F)F)nc1] | 1H NMR (400 MHz, CDCl3) δ 8.77 (d, J = 2.4 Hz, 1 H), 8.47 (s, 1 H), 8.01 (dd, J = 2.4, 8.4 Hz, 1 H), 7.69 (d, J = 8.4 Hz, 1 H), 6.91 (t, J = 51.6 Hz, 1 H), 6.63 (t, J = 55.2 Hz, 1 H), 5.32 (s, 2 H), 3.21 (q, J = 7.2 Hz, 2 H), 1.45 (t, J = 7.6 Hz, 3 H). LCMS RT = 2.547 min, m/z = 452.1 | 0.065 |
| I-101 | 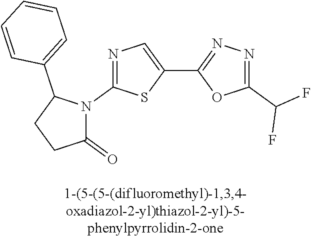 [SMILES: O=C1CCC(c2ccccc2)N1c1ncc(-c2nnc(C(F)F)o2)s1] | 1H NMR (400 MHz, CDCl3) δ 8.08 (s, 1 H), 7.38-7.30 (m, 3 H), 7.18 (d, J = 7.2 Hz, 2 H), 6.88 (t, J = 51.6 Hz, 1 H), 5.80 (d, J = 7.6 Hz, 1 H), 2.93-2.74 (m, 3 H), 2.25-2.20 (m, 1 H). LCMS RT = 2.937 min, m/z = 362.9 | 0.171 |
| I-102 | 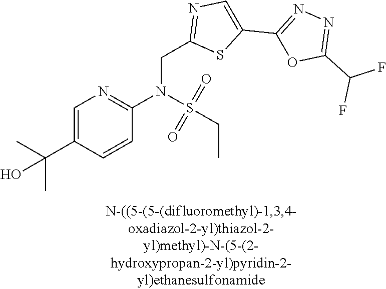 [SMILES: CCS(=O)(=O)N(Cc1ncc(-c2nnc(C(F)F)o2)s1)c1ccc(C(C)(C)O)cn1] | 1H NMR (400 MHz, CDCl3) δ 8.55 (d, J = 2.0 Hz, 1 H), 8.38 (s, 1 H), 7.86 (d, J = 6.8 Hz, 1 H), 7.60 (d, J = 8.8 Hz, 1 H), 6.89 (t, J = 51.6 Hz, 1 H), 5.58 (s, 2 H), 3.29 (q, J = 7.6 Hz , 2 H), 1.61 (s, 6 H), 1.39 (t, J = 7.6 Hz, 3 H). LCMS RT = 1.017 min, m/z = 459.9 | 0.137 |
| I-103 | 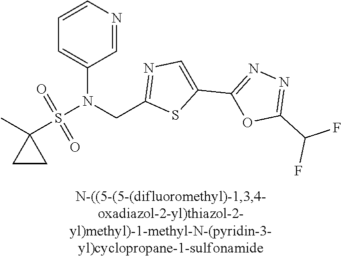 [SMILES: CC1(S(=O)(=O)N(Cc2ncc(-c3nnc(C(F)F)o3)s2)c2cccnc2)CC1] | 1H NMR (400 MHz, CDCl3) δ 8.74 (d, J = 2.4 Hz, 1 H), 8.58 (d, J = 3.6 Hz, 1 H), 8.35 (s, 1 H), 7.87-7.81 (m, 1 H), 7.38-7.31 (m, 1 H), 6.90 (t, J = 51.6 Hz, 1 H), 5.35 (s, 2 H), 1.59 (s, 3 H), 1.22-1.15 (m, 2 H), 0.79-0.72 (m, 2 H). LCMS RT = 0.956 min, m/z = 427.9 | 0.013 |
| I-104 | 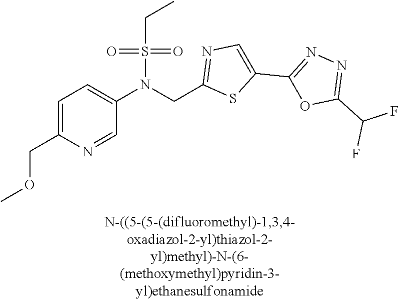 [SMILES: CCS(=O)(=O)N(Cc1ncc(-c2nnc(C(F)F)o2)s1)c1ccc(COC)nc1] | 1H NMR (400 MHz, CDCl3) δ 8.63 (d, J = 2.4 Hz, 1 H), 8.38 (s, 1 H), 7.83 (dd, J = 2.4, 8.0 Hz, 1 H), 7.48 (d, J = 8.4 Hz, 1 H), 6.91 (t, J = 51.6 Hz, 1 H), 5.29 (s, 2 H), 4.57 (s, 2 H), 3.49 (s, 3 H), 3.20 (q, J = 7.6 Hz, 2 H), 1.46 (t, J = 7.6 Hz, 3 H). LCMS RT = 0.789 min, m/z = 446.2 | 0.051 |
| I-105 | 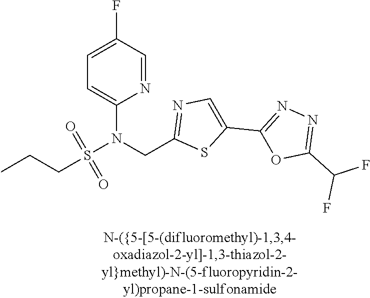 [SMILES: CCCS(=O)(=O)N(Cc1ncc(-c2nnc(C(F)F)o2)s1)c1ccc(F)cn1] | ¹H NMR (400 MHz, CDCl₃) δ 8.37 (s, 1 H), 8.28 (d, J = 3.2 Hz, 1 H), 7.69-7.63 (m, 1 H), 7.51- 7.43 (m, 1 H), 6.89 (t, J = 51.6 Hz, 1 H), 5.52 (s, 2 H), 3.20- 3.14 (m, 2 H), 1.91-1.83 (m, 2 H), 1.05 (t, J = 7.6 Hz, 3 H). LCMS RT = 0.684 min, m/z = 433.9 | 0.0268 |
| I-106 | 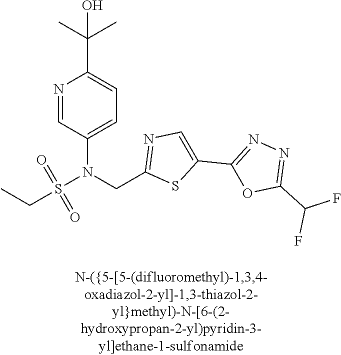 [SMILES: CCS(=O)(=O)N(Cc1ncc(-c2nnc(C(F)F)o2)s1)c1ccc(C(C)(C)O)nc1] | ¹H NMR (400 MHz, CDCl₃) δ 8.55 (s, 1 H), 8.38 (s, 1 H), 7.85 (d, J = 8.8 Hz, 1 H), 7.60 (d, J = 8.4 Hz, 1 H), 6.89 (t, J = 52.0 Hz, 1 H), 5.58 (s, 2 H), 3.30 (q, J = 7.2 Hz, 2 H), 2.63 (s, 1 H), 1.39 (t, J = 7.6 Hz, 3 H), 1.30-1.23 (m, 6 H). LCMS RT = 1.467 min, m/z = 460.2 | 0.480 |
| I-107 | 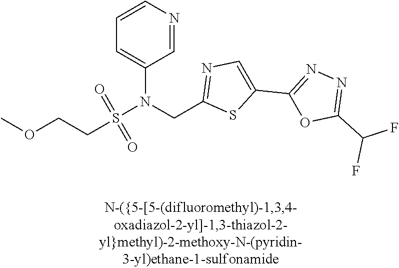 [SMILES: COCCS(=O)(=O)N(Cc1ncc(-c2nnc(C(F)F)o2)s1)c1cccnc1] | ¹H NMR (400 MHz, CDCl₃) δ 8.80 (s, 1 H), 8.57 (d, J = 3.6 Hz, 1 H), 8.34 (s, 1 H), 7.91-7.88 (m, 1 H), 7.36-7.33 (m, 1 H), 6.90 (t, J = 51.6 Hz, 1 H), 5.24 (s, 2 H), 3.87 (t, J = 5.2 Hz, 2 H), 3.51 (s, 3 H), 3.35 (t, J = 5.2 Hz, 2 H). LCMS RT = 1.284 min, m/z = 431.9 | 0.0212 |
| I-108 | 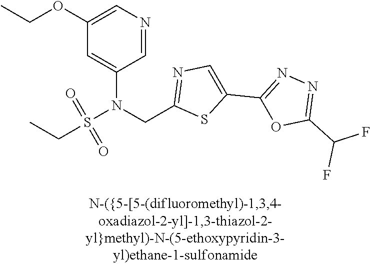 [SMILES: CCOc1cncc(N(Cc2ncc(-c3nnc(C(F)F)o3)s2)S(=O)(=O)CC)c1] | ¹H NMR (400 MHz, CDCl₃) δ 8.40 (s, 1 H), 8.32-8.27 (m, 2 H), 7.42-7.38 (m, 1 H), 6.92 (t, J = 51.6 Hz, 1 H), 5.31 (s, 2 H), 4.10 (q, J = 6.8 Hz, 2 H), 3.22 (q, J = 7.2 Hz, 2 H), 1.49-1.43 (m, 6 H). LCMS RT = 0.604 min, m/z = 446.0 | 0.0138 |
| I-109 | 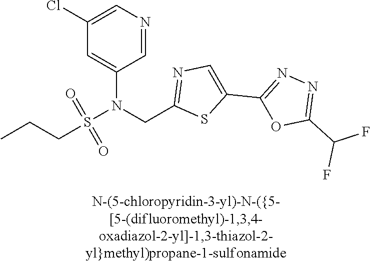 [SMILES: CCCS(=O)(=O)N(Cc1ncc(-c2nnc(C(F)F)o2)s1)c1cncc(Cl)c1] | ¹H NMR (400 MHz, CDCl₃) δ 8.60 (d, J = 2.0 Hz, 1 H), 8.54 (s, 1 H), 8.40 (s, 1 H), 7.88 (t, J = 2.4 Hz, 1 H), 6.91 (t, J = 51.6 Hz, 1 H), 5.27 (s, 2 H), 3.22-3.00 (m, 2 H), 1.99-1.70 (m, 2 H), 1.09 (t, J = 7.6 Hz, 3 H). LCMS RT = 0.668 min, m/z = 449.9 | 0.00392 |
| I-110 | 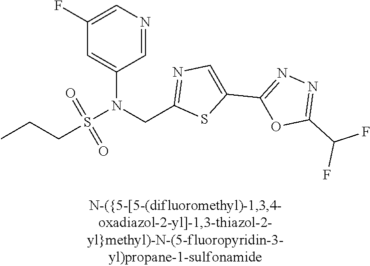 [SMILES: CCCS(=O)(=O)N(Cc1ncc(-c2nnc(C(F)F)o2)s1)c1cncc(F)c1] | ¹H NMR (400 MHz, CDCl₃) δ 8.56 (s, 1 H), 8.46 (d, J = 2.4 Hz, 1 H), 8.40 (s, 1 H), 7.67-7.61 (m, 1 H), 6.91 (t, J = 51.6 Hz, 1 H), 5.29 (s, 2 H), 3.19-3.09 (m, 2 H), 1.97-1.85 (m, 2 H), 1.08 (t, J = 7.2 Hz, 3 H). LCMS RT = 2.144 min, m/z = 434.2 | 0.0104 |
| I-111 | 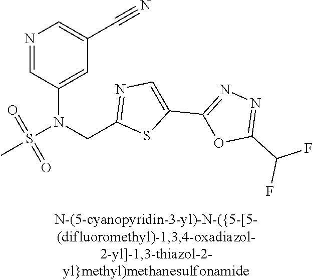 [SMILES: CS(=O)(=O)N(Cc1ncc(-c2nnc(C(F)F)o2)s1)c1cncc(C#N)c1] | ¹H NMR (400 MHz, CDCl₃) δ 8.98 (d, J = 2.4 Hz, 1 H), 8.84 (s, 1 H), 8.45 (s, 1 H), 8.20 (s, 1 H), 6.93 (t, J = 51.2 Hz, 1 H), 5.29 (s, 2 H), 3.11 (s, 3 H). LCMS RT = 1.456 min, m/z = 412.8 | 0.0374 |
| I-112 | 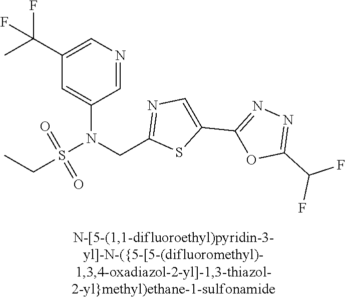 [SMILES: CCS(=O)(=O)N(Cc1ncc(-c2nnc(C(F)F)o2)s1)c1cncc(C(C)(F)F)c1] | ¹H NMR (400 MHz, CDCl₃) δ 8.79 (s, 1 H), 8.72 (s, 1 H), 8.40 (s, 1 H), 7.96 (s, 1 H), 6.91 (t, J = 52.0 Hz, 1 H), 5.30 (s, 2 H), 3.21 (q, J = 7.2 Hz, 2 H), 1.97 (t, J = 18.4 Hz, 3 H), 1.46 (t, J = 7.6 Hz, 3 H). LCMS RT = 1.138 min, m/z = 466.2 | 0.0101 |
| I-113 | 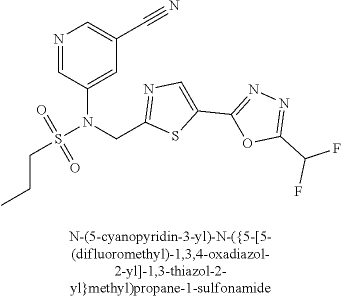 [SMILES: CCCS(=O)(=O)N(Cc1ncc(-c2nnc(C(F)F)o2)s1)c1cncc(C#N)c1] | ¹H NMR (400 MHz, CDCl₃) δ 8.95 (d, J = 2.4 Hz, 1 H), 8.82 (d, J = 2.0 Hz, 1 H), 8.42 (s, 1 H), 8.20 - 8.12 (m, 1 H), 6.92 (t, J = 51.6 Hz, 1 H), 5.29 (s, 2 H), 3.19- 3.08 (m, 2 H), 1.98-1.85 (m, 2 H), 1.09 (t, J = 7.6 Hz, 3 H). LCMS RT = 1.560 min, m/z = 441.2 | 0.0429 |
| I-114 | 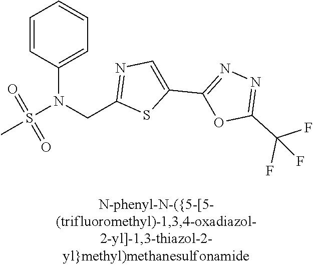 [SMILES: CS(=O)(=O)N(Cc1ncc(-c2nnc(C(F)(F)F)o2)s1)c1ccccc1] | ¹H NMR (400 MHz, CDCl₃) δ 8.39 (s, 1 H), 7.48-7.38 (m, 5 H), 5.28 (s, 2 H), 3.06 (s, 3 H). LCMS RT = 2.568 min, m/z = 404.8 | 0.354 |
| I-115 | 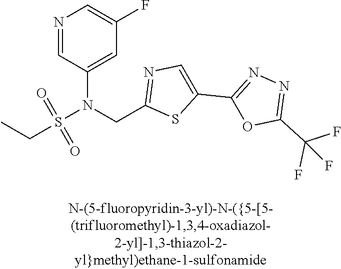 [SMILES: CCS(=O)(=O)N(Cc1ncc(-c2nnc(C(F)(F)F)o2)s1)c1cncc(F)c1] | ¹H NMR (400 MHz, CDCl₃) δ 8.57 (s, 1 H), 8.47 (d, J = 2.4 Hz, 1 H), 8.42 (s, 1 H), 7.68-7.63 (m, 1 H), 5.31 (s, 2 H), 3.22 (q, J = 7.2 Hz, 2 H), 1.46 (t, J = 7.2 Hz, 3 H). LCMS RT = 0.904 min, m/z = 437.9 | 0.022 |
| I-116 | 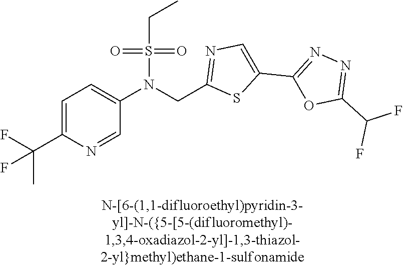 [SMILES: CCS(=O)(=O)N(Cc1ncc(-c2nnc(C(F)F)o2)s1)c1ccc(C(C)(F)F)nc1] | ¹H NMR (400 MHz, CDCl₃) δ 8.74 (d, J = 2.4 Hz, 1 H), 8.39 (s, 1 H), 7.98-7.92 (m, 1 H), 7.70 (d, J = 8.4 Hz, 1 H), 6.91 (t, J = 51.6 Hz, 1 H), 5.32 (s, 2 H), 3.22 (q, J = 7.6 Hz, 2 H), 2.01 (t, J = 18.8 Hz, 3 H), 1.46 (t, J = 7.6 Hz, 3 H). LCMS RT = 0.908 min, m/z = 465.9 | 0.0514 |
| I-117 | 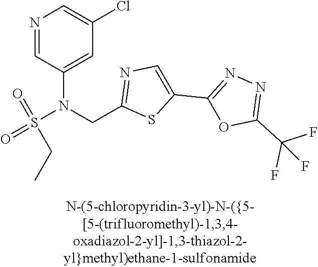 [SMILES: CCS(=O)(=O)N(Cc1ncc(-c2nnc(C(F)(F)F)o2)s1)c1cncc(Cl)c1] | ¹H NMR (400 MHz, CDCl₃) δ 8.61 (d, J = 2.4 Hz, 1 H), 8.55 (d, J = 2.4 Hz, 1 H), 8.42 (s, 1 H), 7.88 (t, J = 2.0 Hz, 1 H), 5.29 (s, 2 H), 3.21 (q, J = 4.0 Hz, 2 H), 1.46 (t, J = 8.0 Hz, 3 H). LCMS RT = 2.591 min, m/z = 453.8 | 0.0245 |
| I-118 | 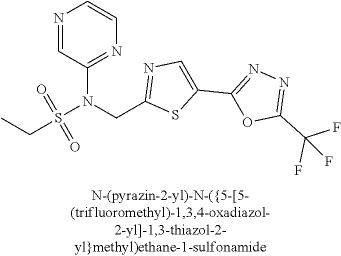 [SMILES: CCS(=O)(=O)N(Cc1ncc(-c2nnc(C(F)(F)F)o2)s1)c1cnccn1] | ¹H NMR (400 MHz, CDCl₃) δ 9.00 (s, 1 H), 8.44-8.37 (m, 3 H), 5.58 (s, 2 H), 3.41 (d, J = 6.8 Hz, 2 H), 1.43 (t, J = 6.8 Hz, 3 H). LCMS RT = 0.895 min, m/z = 420.9 | 0.0278 |
| I-119 | 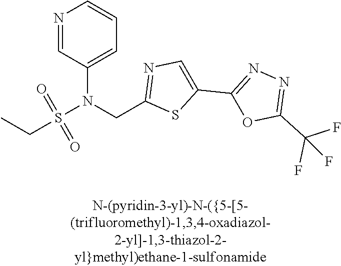 [SMILES: CCS(=O)(=O)N(Cc1ncc(-c2nnc(C(F)(F)F)o2)s1)c1cccnc1] | ¹H NMR (400 MHz, CDCl₃) δ 8.73 (s, 1 H), 8.62-8.60 (m, 1 H), 8.41 (s, 1 H), 7.86 (d, J = 8.0 Hz, 1 H), 7.40-7.35 (m, 1 H), 5.32 (s, 2 H), 3.22 (q, J = 6.8 Hz, 2 H), 1.48 (t, J = 7.2 Hz, 3 H). LCMS RT = 1.897 min, m/z = 420.1 | 0.042 |
| I-120 | 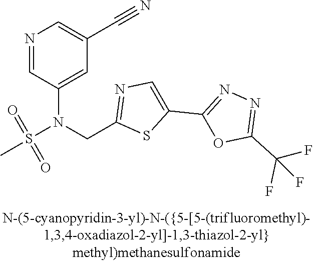 [SMILES: CS(=O)(=O)N(Cc1ncc(-c2nnc(C(F)(F)F)o2)s1)c1cncc(C#N)c1] | ¹H NMR (400 MHz, CDCl₃) δ 8.98 (d, J = 2.8 Hz, 1 H), 8.85 (s, 1 H), 8.46 (s, 1 H), 8.20 (t, J = 2.0 Hz, 1 H), 5.30 (s, 2 H), 3.11 (s, 3 H). LCMS RT = 2.105 min, m/z = 431.1 | 0.111 |
| I-121 | 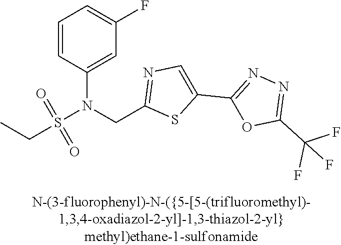 [SMILES: CCS(=O)(=O)N(Cc1ncc(-c2nnc(C(F)(F)F)o2)s1)c1cccc(F)c1] | ¹H NMR (400 MHz, CDCl₃) δ 8.40 (s, 1 H), 7.41-7.36 (m, 1 H), 7.28 (s, 1 H), 7.25-7.22 (m, 1 H), 7.09-7.04 (m, 1 H), 5.30 (s, 2 H), 3.19 (q, J = 7.6 Hz, 2 H), 1.45 (t, J = 7.2 Hz, 3 H). LCMS RT = 1.978 min, m/z = 437.1 | 0.0839 |
| I-122 | 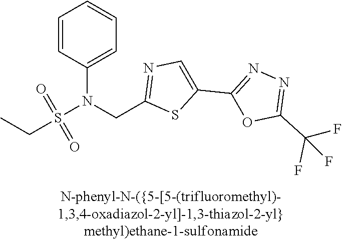 [SMILES: CCS(=O)(=O)N(Cc1ncc(-c2nnc(C(F)(F)F)o2)s1)c1ccccc1] | ¹H NMR (400 MHz, CDCl₃) δ 8.38 (s, 1 H), 7.50-7.30 (m, 5 H), 5.31 (s, 2 H), 3.17 (q, J = 7.2 Hz, 2 H), 1.45 (t, J = 7.2 Hz, 3 H). LCMS RT = 0.973 min, m/z = 419.0 | 0.0249 |
| I-123 | 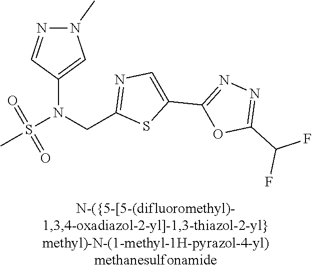 [SMILES: Cn1cc(N(Cc2ncc(-c3nnc(C(F)F)o3)s2)S(C)(=O)=O)cn1] | ¹H NMR (400 MHz, CDCl₃) δ 8.42 (s, 1 H), 7.57 (s, 1 H), 7.48 (s, 1 H), 6.91 (t, J = 51.6 Hz, 1 H), 5.14 (s, 2 H), 3.88 (s, 3 H), 3.03 (s, 3 H). LCMS RT = 1.057 min, m/z = 390.9 | 0.0388 |
| I-124 | 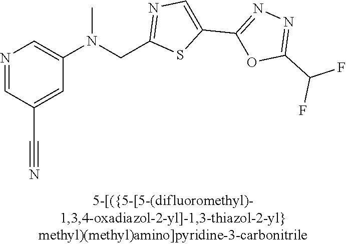 [SMILES: CN(Cc1ncc(-c2nnc(C(F)F)o2)s1)c1cncc(C#N)c1] | ¹H NMR (400 MHz, CDCl₃) δ 8.46 (s, 1 H), 8.41 (s, 1 H), 8.33 (s, 1 H), 7.25 (s, 1 H), 6.90 (t, J = 51.6 Hz, 1 H), 4.92 (s, 2 H), 3.27 (s, 3 H). LCMS RT = 0.971 min, m/z = 349.0 | 0.0636 |
| I-125 | 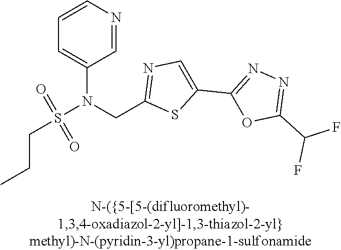 [SMILES: CCCS(=O)(=O)N(Cc1ncc(-c2nnc(C(F)F)o2)s1)c1cccnc1] | ¹H NMR (400 MHz, CDCl₃) δ 8.71 (d, J = 2.4 Hz, 1 H), 8.60- 8.56 (m, 1 H), 8.38 (s, 1 H), 7.85- 7.82 (m, 1 H), 7.39-7.32 (m, 1 H), 6.91 (t, J = 51.6 Hz, 1 H), 5.29 (s, 2 H), 3.18-3.11 (m, 2 H), 1.98-1.89 (m, 2 H), 1.09 (t, J = 7.2 Hz, 3 H). LCMS RT = 0.776 min, m/z = 416.1 | 0.0599 |
| I-126 | 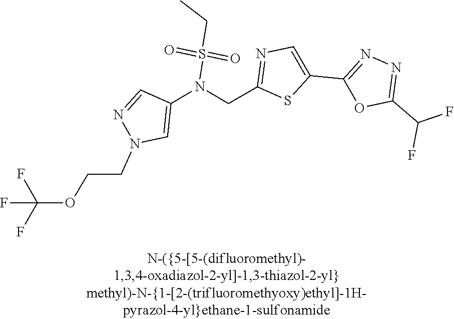 [SMILES: CCS(=O)(=O)N(Cc1ncc(-c2nnc(C(F)F)o2)s1)c1cnn(CCOC(F)(F)F)c1] | ¹H NMR (400 MHz, CDCl₃) δ 8.39 (s, 1 H), 7.64 (s, 1 H), 7.57 (s, 1 H), 6.91 (t, J = 51.6 Hz, 1 H), 5.16 (s, 2 H), 4.36-4.33 (m, 2 H), 4.30-4.23 (m, 2 H), 3.15 (q, J = 7.6 Hz, 2 H), 1.40 (t, J = 7.2 Hz, 3 H). LCMS RT = 1.043 min, m/z = 502.9 | 0.134 |
| I-127 | 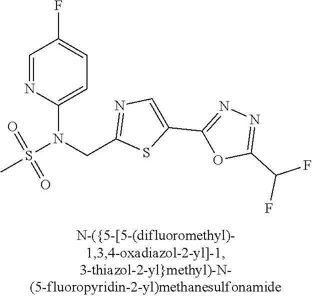 [SMILES: CS(=O)(=O)N(Cc1ncc(-c2nnc(C(F)F)o2)s1)c1ccc(F)cn1] | ¹H NMR (400 MHz, CDCl₃) δ 8.37 (s, 1 H), 8.29 (d, J = 3.2 Hz, 1 H), 7.67-7.61 (m, 1 H), 7.53- 7.46 (m, 1 H), 6.90 (t, J = 51.6 Hz, 1 H), 5.50 (s, 2 H), 3.10 (s, 3 H). LCMS RT = 0.854 min, m/z = 406.1 | 0.0215 |
| I-128 | 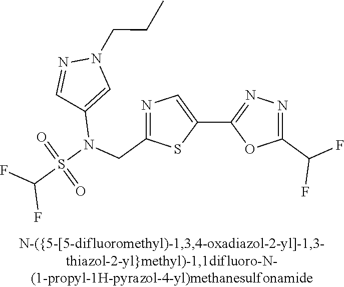 [SMILES: CCCn1cc(N(Cc2ncc(-c3nnc(C(F)F)o3)s2)S(=O)(=O)C(F)F)cn1] | ¹H NMR (400 MHz, CDCl₃) δ 8.42 (s, 1 H), 7.49 (d, J = 4.4 Hz, 2 H), 6.92 (t, J = 51.6 Hz, 1 H), 6.44 (t, J = 53.2 Hz, 1 H), 5.21 (s, 2 H), 4.02 (t, J = 7.2 Hz, 2 H), 1.94-1.78 (m, 2 H), 0.88 (t, J = 7.6 Hz, 3 H). LCMS RT = 0.901 min, m/z = 455.1 | 0.251 |
| I-129 | 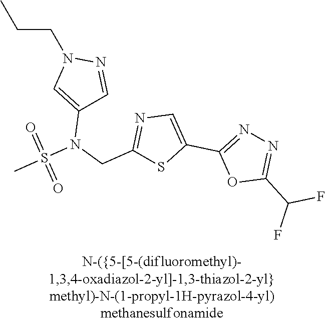 [SMILES: CCCn1cc(N(Cc2ncc(-c3nnc(C(F)F)o3)s2)S(C)(=O)=O)cn1] | ¹H NMR (400 MHz, CDCl₃) δ 8.41 (s, 1 H), 7.58 (s, 1 H), 7.49 (s, 1 H), 6.91 (t, J = 51.6 Hz, 1 H), 5.14 (s, 2 H), 4.03 (t, J = 7.2 Hz, 2 H), 3.03 (s, 3 H), 1.92 - 1.81 (m, 2 H), 0.90 (t, J = 7.2 Hz, 3 H). LCMS RT = 0.795 min, m/z = 418.9 | 0.118 |
| I-130 | 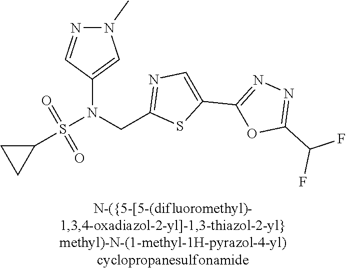 [SMILES: Cn1cc(N(Cc2ncc(-c3nnc(C(F)F)o3)s2)S(=O)(=O)C2CC2)cn1] | ¹H NMR (400 MHz, CDCl₃) δ 8.39 (s, 1 H), 7.56 (s, 1 H), 7.52 (s, 1 H), 6.91 (t, J = 51.6 Hz, 1 H), 5.15 (s, 2 H), 3.87 (s, 3 H), 2.58-2.50 (m, 1 H), 1.20-1.15 (m, 2 H), 1.07-1.01 (m, 2 H). LCMS RT = 1.320 min, m/z = 416.8 | 0.0348 |
| I-131 | 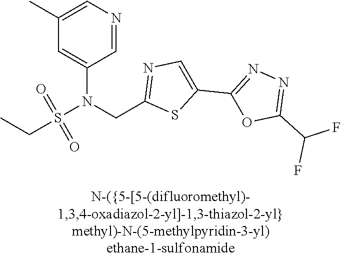 [SMILES: CCS(=O)(=O)N(Cc1ncc(-c2nnc(C(F)F)o2)s1)c1cncc(C)c1] | ¹H NMR (400 MHz, CDCl₃) δ 8.50 (d, J = 2.4 Hz, 1 H), 8.41 (s, 1 H), 8.38 (s, 1 H), 7.65 (s, 1 H), 6.91 (t, J = 51.6 Hz, 1 H), 5.29 (s, 2 H), 3.20 (q, J = 7.6 Hz, 2 H), 2.37 (s, 3 H), 1.47 (t, J = 7.6 Hz, 3 H). LCMS RT = 0.714 min, m/z = 416.2 | 0.013 |
| I-132 | 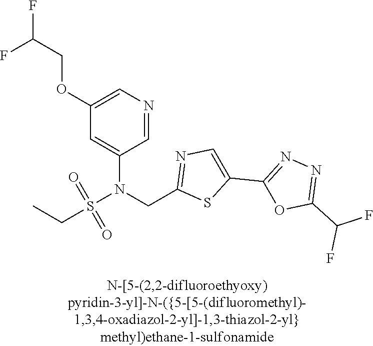 [SMILES: CCS(=O)(=O)N(Cc1ncc(-c2nnc(C(F)F)o2)s1)c1cncc(OCC(F)F)c1] | ¹H NMR (400 MHz, CDCl₃) δ 8.40 (s, 2 H), 8.32 (d, J = 2.4 Hz, 1 H), 7.45 (t, J = 2.4 Hz, 1 H), 6.91 (t, J = 51.6 Hz, 1 H), 6.25- 5.96 (m, 1 H), 5.30 (s, 2 H), 4.30- 4.20 (m, 2 H), 3.21 (q, J = 7.2 Hz, 2 H), 1.46 (t, J = 7.6 Hz, 3 H). LCMS RT = 0.857 min, m/z = 482.2 | 0.00964 |
| I-133 | 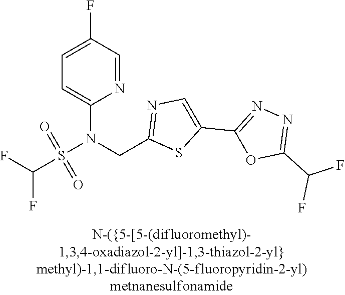 [SMILES: O=S(=O)(C(F)F)N(Cc1ncc(-c2nnc(C(F)F)o2)s1)c1ccc(F)cn1] | ¹H NMR (400 MHz, CDCl₃) δ 8.39 (s, 1 H), 8.30 (d, J = 2.8 Hz, 1 H), 7.58-7.55 (m, 1 H), 7.54- 7.48 (m, 1 H), 7.03 (t, J = 51.6 Hz, 1 H), 6.33 (t, J = 53.6 Hz, 1 H), 5.55 (s, 2 H). LCMS RT = 0.954 min, m/z = 441.9 | 0.0857 |
| I-134 | 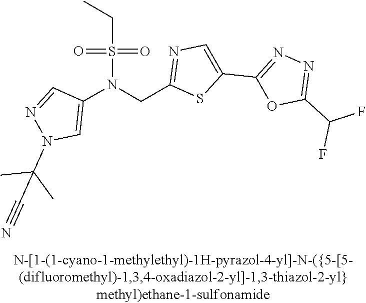 [SMILES: CCS(=O)(=O)N(Cc1ncc(-c2nnc(C(F)F)o2)s1)c1cnn(C(C)(C)C#N)c1] | 1H NMR (400 MHz, CDCl3) 8 8.42 (s, 1 H), 7.83 (s, 1 H), 7.63 (s, 1 H), 6.91 (t, J = 51.6 Hz, 1 H), 5.17 (s, 2 H), 3.17 (q, J = 7.2 Hz, 2 H), 1.99 (s, 6 H), 1.42 (t, J = 7.2 Hz, 3 H). LCMS RT = 1.012 min, m/z = 458.1 | 0.121 |
| I-135 | 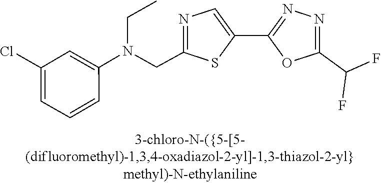 [SMILES: CCN(Cc1ncc(-c2nnc(C(F)F)o2)s1)c1cccc(Cl)c1] | ¹H NMR (400 MHz, CDCl₃) δ 8.45 (s, 1 H), 7.14 (t, J = 8.0 Hz, 1 H), 6.88 (t, J = 51.6 Hz, 1 H), 6.76-6.74 (m, 2 H), 6.64-6.60 (m, 1 H), 4.79 (s, 2 H), 3.58 (q, J = 7.2 Hz, 2 H), 1.30 (t, J = 7.2 Hz, 3 H). LCMS RT = 1.098 min, m/z = 370.9 | 0.0531 |
| I-136 | 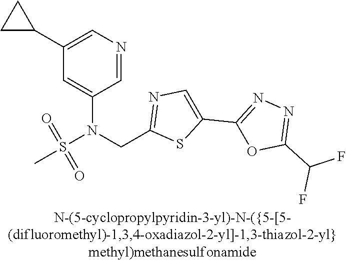 [SMILES: CS(=O)(=O)N(Cc1ncc(-c2nnc(C(F)F)o2)s1)c1cncc(C2CC2)c1] | ¹H NMR (400 MHz, CDCl₃) δ 8.46 (s, 1 H), 8.41 (s, 1 H), 8.37 (s, 1 H), 7.46 (s, 1 H), 6.91 (t, J = 51.6 Hz, 1 H), 5.25 (s, 2 H), 3.09 (s, 3 H), 1.96-1.89 (m, 1 H), 1.14-1.06 (m, 2 H), 0.79-0.71 (m, 2 H). LCMS RT = 0.743 min, m/z = 427.9 | 0.013 |
| I-137 | 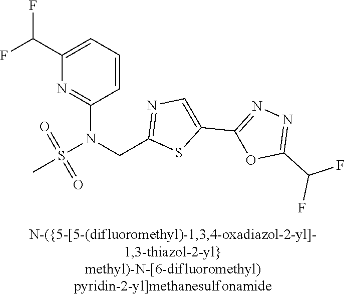 [SMILES: CS(=O)(=O)N(Cc1ncc(-c2nnc(C(F)F)o2)s1)c1cccc(C(F)F)n1] | ¹H NMR (400 MHz, CDCl₃) δ 8.38 (s, 1 H), 7.90 (t, J = 8.0 Hz, 1 H), 7.77 (d, J = 8.4 Hz, 1 H), 7.48 (d, J = 7.6 Hz, 1 H), 6.89 (t, J = 51.6 Hz, 1 H), 6.54 (t, J = 55.2 Hz, 1 H), 5.59 (s, 2 H), 3.23 (s, 3 H). LCMS RT = 0.899 min, m/z = 437.9 | 0.0107 |
| I-138 | 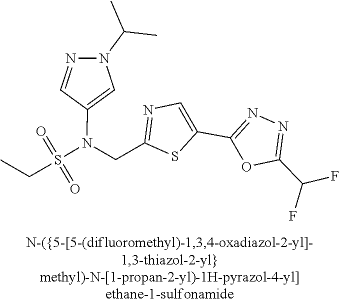 [SMILES: CCS(=O)(=O)N(Cc1ncc(-c2nnc(C(F)F)o2)s1)c1cnn(C(C)C)c1] | ¹H NMR (400 MHz, CDCl₃) δ 8.37 (s, 1 H), 7.57 (s, 1 H), 7.46 (s, 1 H), 6.87 (t, J = 51.2 Hz, 1 H), 5.12 (s, 2 H), 4.44-4.34 (m, 1 H), 3.13 (q, J = 7.2 Hz, 2 H), 1.45 (d, J = 6.8 Hz, 6 H), 1.39 (t, J = 7.2 Hz, 3 H). LCMS RT = 1.141 min, m/z = 433.2 | 0.043 |
| I-139 | 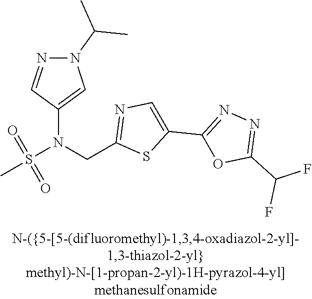 [SMILES: CC(C)n1cc(N(Cc2ncc(-c3nnc(C(F)F)o3)s2)S(C)(=O)=O)cn1] | ¹H NMR (400 MHz, CDCl₃) δ 8.39 (s, 1 H), 7.58 (s, 1 H), 7.46 (s, 1 H), 6.88 (t, J = 51.6 Hz, 1 H), 5.11 (s, 2 H), 4.45-4.35 (m, 1 H), 3.00 (s, 3 H), 1.46 (d, J = 6.8 Hz, 6 H). LCMS RT = 0.997 min, m/z = 419.2 | 0.118 |
| I-140 | 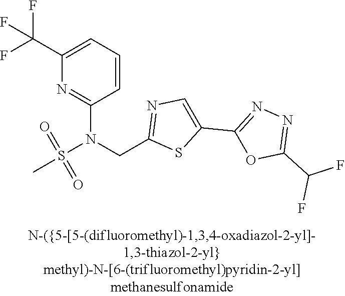 [SMILES: CS(=O)(=O)N(Cc1ncc(-c2nnc(C(F)F)o2)s1)c1cccc(C(F)(F)F)n1] | ¹H NMR (400 MHz, CDCl₃) δ 8.37 (s, 1 H), 7.95-7.85 (m, 2 H), 7.51 (d, J = 7.2 Hz, 1 H), 6.90 (t, J = 51.6 Hz, 1 H), 5.61 (s, 2 H), 3.29 (s, 3 H). LCMS RT = 1.112 min, m/z = 455.8 | 0.0576 |
| I-141 | 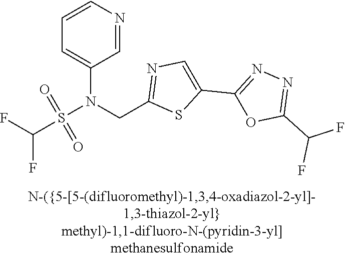 [SMILES: O=S(=O)(C(F)F)N(Cc1ncc(-c2nnc(C(F)F)o2)s1)c1cccnc1] | ¹H NMR (400 MHz, CDCl₃) δ 8.70-8.60 (m, 2 H), 8.39 (s, 1 H), 7.75 (d, J = 6.8 Hz, 1 H), 7.39- 7.36 (m, 1 H), 6.92 (t, J = 51.6 Hz, 1 H), 6.49 (t, J = 53.2 Hz, 1 H), 5.35 (s, 2 H). LCMS RT = 0.965 min, m/z = 423.8 | 0.0655 |
| I-142 | 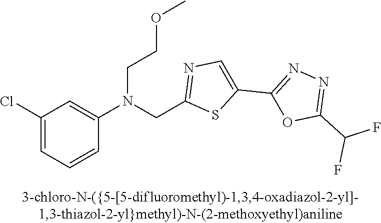 [SMILES: COCCN(Cc1ncc(-c2nnc(C(F)F)o2)s1)c1cccc(Cl)c1] | ¹H NMR (400 MHz, CDCl₃) δ 8.44 (s, 1 H), 7.13 (t, J = 8.0 Hz, 1 H), 6.88 (t, J = 89.2 Hz, 1 H), 6.80-6.70 (m, 2 H), 6.68-6.55 (m, 1H), 4.94 (s, 2 H), 3.74-3.71 (m, 2 H), 3.70-3.65 (m, 2 H), 3.38 (s, 3 H). LCMS RT = 1.055 min, m/z = 400.9 | 0.0245 |
| I-143 | 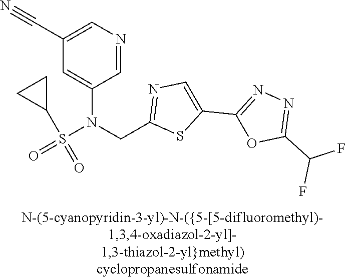 [SMILES: N#Cc1cncc(N(Cc2ncc(-c3nnc(C(F)F)o3)s2)S(=O)(=O)C2CC2)c1] | 1H NMR (400 MHz, CDCl3) 8 8.99 (d, J = 2.0 Hz, 1 H), 8.83 (s, 1 H), 8.41 (s, 1 H), 8.18 (t, J = 2.0 Hz, 1 H), 6.92 (t, J = 51.6 Hz, 1H), 5.31 (s, 2 H), 2.59-2.50 (m, 1 H), 1.13-1.08 (m, 4 H). LCMS RT = 0.834 min, m/z = 439.1 | 0.0153 |
| I-144 | 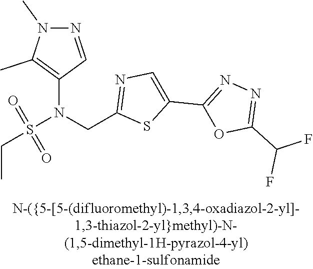 [SMILES: CCS(=O)(=O)N(Cc1ncc(-c2nnc(C(F)F)o2)s1)c1cnn(C)c1C] | ¹H NMR (400 MHz, CDCl₃) δ 8.39 (s, 1 H), 7.38 (s, 1 H), 6.91 (t, J = 51.6 Hz, 1 H), 5.11 (s, 2 H), 3.76 (s, 3 H), 3.20 (q, J = 7.2 Hz, 2 H), 2.25 (s, 3 H), 1.47 (t, J = 7.4 Hz, 3 H). LCMS RT = 0.751 min, m/z = 419.0 | 0.102 |
| I-145 | 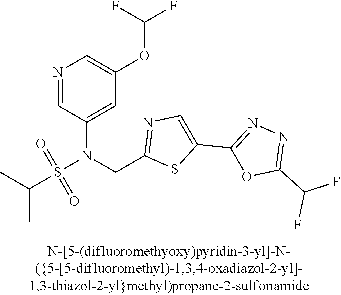 [SMILES: CC(C)S(=O)(=O)N(Cc1ncc(-c2nnc(C(F)F)o2)s1)c1cncc(OC(F)F)c1] | 1H NMR (400 MHz, CDCl3) δ 8.61 (d, J = 2.0 Hz, 1 H), 8.45 (d, J = 2.4 Hz, 1 H), 8.38 (s, 1 H), 7.68 (t, J = 2.0 Hz, 1 H), 6.91 (t, J = 51.6 Hz, 1 H), 6.58 (t, J = 72.0 Hz, 1 H), 5.31 (s, 2 H), 3.40- 3.29 (m, 1 H), 1.45 (d, J = 6.8 Hz, 6 H). LCMS RT = 1.625 min, m/z = 482.2 | 0.0102 |
| I-146 | 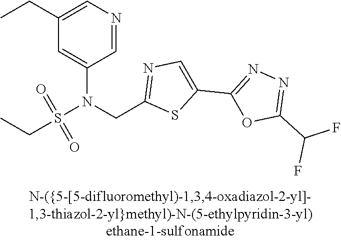 [SMILES: CCc1cncc(N(Cc2ncc(-c3nnc(C(F)F)o3)s2)S(=O)(=O)CC)c1] | 1H NMR (400 MHz, CD3OD) δ 8.59 (s, 1 H), 8.40 (s, 2 H), 8.02 (s, 1 H), 7.19 (t, J = 51.2 Hz, 1 H), 5.40 (s, 2 H), 3.40-3.30 (m, 2 H), 2.73 (q, J = 7.6 Hz, 2 H), 1.39 (t, J = 7.6 Hz, 3 H), 1.25 (t, J = 7.6 Hz, 3 H). LCMS RT = 1.215 min, m/z = 430.2 | 0.0162 |
| I-147 | 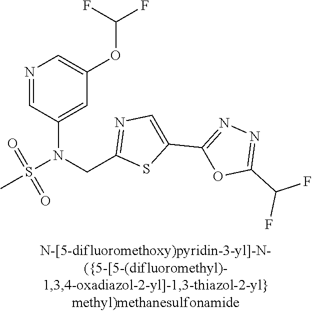 [SMILES: CS(=O)(=O)N(Cc1ncc(-c2nnc(C(F)F)o2)s1)c1cncc(OC(F)F)c1] | 1H NMR (400 MHz, CDCl3) δ 8.62 (s, 1 H), 8.49 (s, 1 H), 8.43 (s, 1 H), 7.69 (s, 1 H), 6.92 (t, J = 51.6 Hz, 1 H), 6.59 (t, J = 72.0 Hz, 1 H), 5.28 (s, 2 H), 3.12 (s, 3 H). LCMS RT = 1.512 min, m/z = 453.8 | 0.019 |
| I-148 | 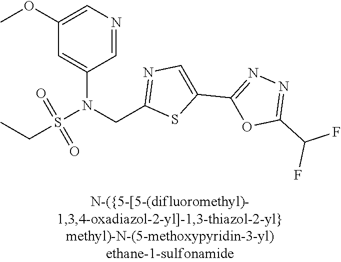 [SMILES: CCS(=O)(=O)N(Cc1ncc(-c2nnc(C(F)F)o2)s1)c1cncc(OC)c1] | 1H NMR (400 MHz, CDCl3) δ ppm 8.39 (s, 1 H), 8.30-8.28 (m, 2 H), 7.38 (s, 1 H), 6.91 (t, J = 51.6 Hz, 1 H), 5.30 (s, 2 H), 3.87 (s, 3 H), 3.21 (q, J = 7.6 Hz, 2 H), 1.46 (t, J = 7.6 Hz, 3 H). LCMS RT = 1.360 min, m/z = 431.9 [M + H]+ | 0.0192 |
| I-149 | 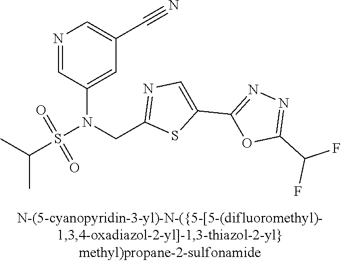 [SMILES: CC(C)S(=O)(=O)N(Cc1ncc(-c2nnc(C(F)F)o2)s1)c1cncc(C#N)c1] | 1H NMR (400 MHz, CDCl3) δ 8.95 (d, J = 2.8 Hz, 1 H), 8.80 (s, 1 H), 8.39 (s, 1 H), 8.16 (s, 1 H), 6.92 (t, J = 51.6 Hz, 1 H), 5.30 (s, 2 H), 3.39-3.25 (m, 1 H), 1.45 (d, J = 6.8 Hz, 6 H). LCMS RT = 1.584 min, m/z = 440.9 | 0.0231 |
| I-150 | 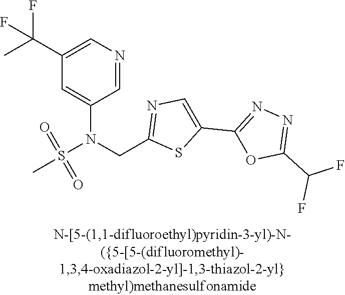 [SMILES: CC(F)(F)c1cncc(N(Cc2ncc(-c3nnc(C(F)F)o3)s2)S(C)(=O)=O)c1] | 1H NMR (400 MHz, CDCl3) δ 8.80 (s, 1 H), 8.75 (s, 1 H), 8.43 (s, 1 H), 7.98 (s, 1 H), 6.92 (t, J = 51.6 Hz, 1 H), 5.29 (s, 2 H), 3.12 (s, 3 H), 1.98 (t, J = 18.0 Hz, 3 H). LCMS RT = 1.554 min, m/z = 451.8 | 0.0255 |
| I-151 | 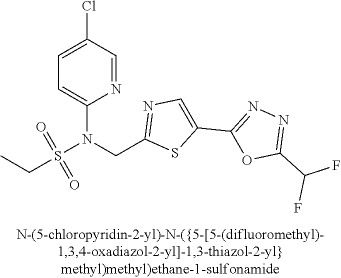 [SMILES: CCS(=O)(=O)N(Cc1ncc(-c2nnc(C(F)F)o2)s1)c1ccc(Cl)cn1] | 1H NMR (400 MHz, CDCl3) δ 8.37 (d, J = 4.0 Hz, 2 H), 7.74- 7.67 (m, 2 H), 6.90 (t, J = 51.6 Hz, 1 H), 5.56 (s, 2 H), 3.29 (q, J = 7.2 Hz, 2 H), 1.39 (t, J = 7.2 Hz, 3 H). LCMS RT = 1.826 min, m/z = 436.1 | 0.0315 |
| I-152 | 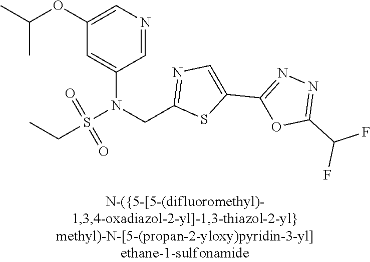 [SMILES: CCS(=O)(=O)N(Cc1ncc(-c2nnc(C(F)F)o2)s1)c1cncc(OC(C)C)c1] | 1H NMR (400 MHz, CDCl3) δ 8.39 (s, 1 H), 8.25 (s, 2 H), 7.35 (s, 1 H), 6.91 (t, J = 51.6 Hz, 1 H), 5.29 (s, 2 H), 4.61-4.55 (m, 1 H), 3.25-3.18 (m, 2 H), 1.61 (s, 3 H), 1.36 (d, J = 4.4 Hz, 6 H). LCMS RT = 1.666 min, m/z = 459.9 | 0.0429 |
| I-153 | 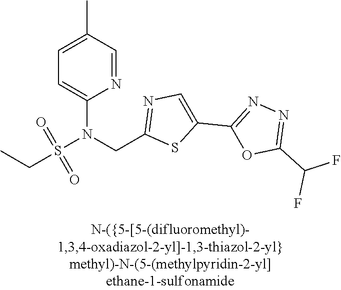 [SMILES: CCS(=O)(=O)N(Cc1ncc(-c2nnc(C(F)F)o2)s1)c1ccc(C)cn1] | 1H NMR (400 MHz, CDCl3) δ 8.22 (s, 1 H), 8.10 (s, 1 H), 7.40 (s, 2 H), 6.75 (t, J = 51.6 Hz, 1 H), 5.42 (s, 2 H), 3.09 (q, J = 7.6 Hz, 2 H), 2.18 (s, 3 H), 1.23 (t, J = 7.2 Hz, 3 H). LCMS RT = 1.666 min, m/z = 416.2 | 0.043 |
| I-155 | 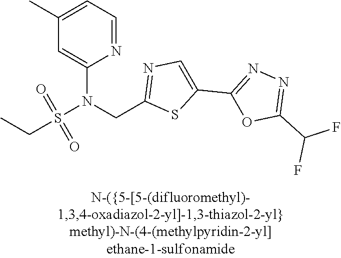 [SMILES: CCS(=O)(=O)N(Cc1ncc(-c2nnc(C(F)F)o2)s1)c1cc(C)ccn1] | 1H NMR (400 MHz, CDCl3) δ 8.39 (s, 1 H), 8.29 (d, J = 4.8 Hz, 1 H), 7.47 (s, 1 H), 7.01 (d, J = 4.8 Hz, 1 H), 6.90 (t, J = 51.6 Hz, 1 H), 5.60 (s, 2 H), 3.28 (q, J = 7.6 Hz, 2 H), 2.40 (s, 3 H), 1.39 (t, J = 7.2 Hz, 3 H). LCMS RT = 1.635 min, m/z = 416.2 | 0.0528 |
| I-156 | 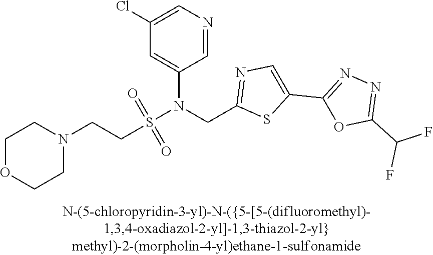 [SMILES: O=S(=O)(CCN1CCOCC1)N(Cc1ncc(-c2nnc(C(F)F)o2)s1)c1cncc(Cl)c1] | 1H NMR (400 MHz, CD3OD) δ ppm 8.68 (d, J = 2.4 Hz, 1 H), 8.52 (d, J = 2.0 Hz, 1 H), 8.44 (s, 1 H), 8.17 (t, J = 2.0 Hz, 1 H), 7.22 (t, J = 52.0 Hz, 1 H), 5.45 (s, 2 H), 3.73-3.70 (m, 4 H), 3.58 (t, J = 7.6 Hz, 2 H), 2.89 (t, J = 6.8 Hz, 2 H), 2.58-2.51 (m, 4 H). LCMS RT = 0.992 min, m/z = 521.2 | 0.00253 |
| I-157 | 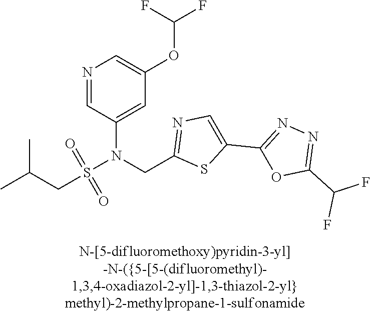 [SMILES: CC(C)CS(=O)(=O)N(Cc1ncc(-c2nnc(C(F)F)o2)s1)c1cncc(OC(F)F)c1] | 1H NMR (400 MHz, CDCl3) δ 8.59 (d, J = 2.0 Hz, 1 H), 8.47 (d, J = 2.0 Hz, 1 H), 8.40 (s, 1 H), 7.67 (t, J = 2.4 Hz, 1 H), 6.91 (t, J = 51.6 Hz, 1 H), 6.59 (t, J = 72.0 Hz, 1 H), 5.28 (s, 2 H), 3.05 (d, J = 8.0 Hz, 2 H), 2.40-2.30 (m, 1 H), 1.12 (d, J = 6.8 Hz, 6 H). LCMS RT = 0.644 min, m/z = 496.1 | 0.00693 |
| I-158 | 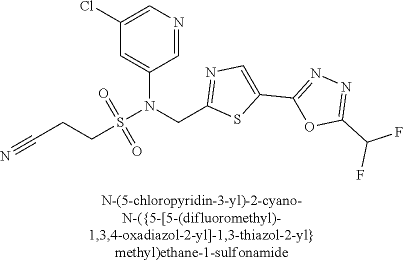 [SMILES: N#CCCS(=O)(=O)N(Cc1ncc(-c2nnc(C(F)F)o2)s1)c1cncc(Cl)c1] | 1H NMR (400 MHz, CDCl3) δ ppm 8.63 (d, J = 2.4 Hz, 1 H), 8.60 (d, J = 2.0 Hz, 1 H), 8.46 (s, 1 H), 7.91 (t, J = 2.4 Hz, 1 H), 6.93 (t, J = 51.6 Hz, 1 H), 5.30 (s, 2 H), 3.54 (t, J = 7.2 Hz, 2 H), 2.98 (t, J = 7.2 Hz, 2 H). LCMS RT = 0.576 min, m/z = 461.1 | 0.00722 |
| I-159 | 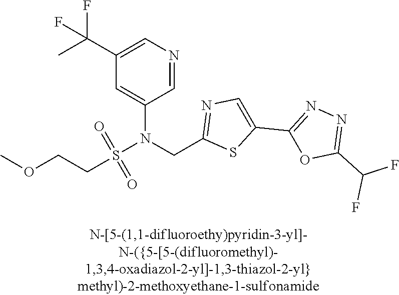 [SMILES: COCCS(=O)(=O)N(Cc1ncc(-c2nnc(C(F)F)o2)s1)c1cncc(C(C)(F)F)c1] | 1H NMR (400 MHz, CDCl3) δ 8.85 (s, 1 H), 8.56 (s, 1 H), 8.72 (s, 1 H), 8.35 (s, 1 H), 8.01 (s, 1 H), 6.91 (t, J = 51.6 Hz, 1 H), 5.26 (s, 2 H), 3.87 (t, J = 5.2 Hz, 2 H), 3.50 (s, 3 H), 3.36 (t, J = 5.2 Hz, 2 H), 1.97 (t, J = 18.4 Hz, 3 H) LCMS RT = 1.701 min, m/z = 495.9 | 0.00994 |
| I-160 | 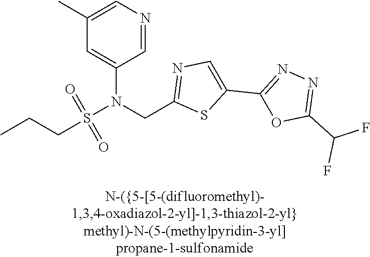 [SMILES: CCCS(=O)(=O)N(Cc1ncc(-c2nnc(C(F)F)o2)s1)c1cncc(C)c1] | 1H NMR (400 MHz, CDCl3) δ 8.49 (d, J = 2.4 Hz, 1 H), 8.41 (s, 1 H), 8.38 (s, 1 H), 7.65 (s, 1 H), 6.91 (t, J = 51.6 Hz, 1 H), 5.27 (s, 2 H), 3.16-3.08 (m, 2 H), 2.37 (s, 3 H), 1.99-1.85 (m, 2 H), 1.09 (t, J = 7.2 Hz, 3 H). LCMS RT = 1.815 min, m/z = 430.2 | 0.0109 |
| I-161 | 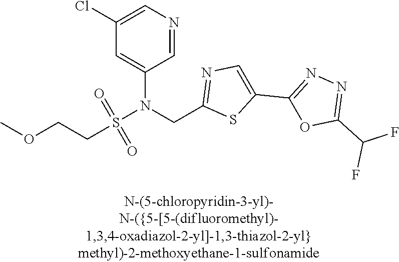 [SMILES: COCCS(=O)(=O)N(Cc1ncc(-c2nnc(C(F)F)o2)s1)c1cncc(Cl)c1] | 1H NMR (400 MHz, CDCl3) δ 8.67 (s, 1 H), 8.53 (s, 1 H), 8.35 (s, 1 H), 7.90 (t, J = 2.0 Hz, 1 H), 6.90 (t, J = 51.6 Hz, 1 H), 5.22 (s, 2 H), 3.86 (t, J = 5.6 Hz, 2 H), 3.49 (s, 3 H), 3.36 (t, J = 5.6 Hz, 2 H). LCMS RT = 1.529 min, m/z = 466.1 | 0.012 |
| I-162 | 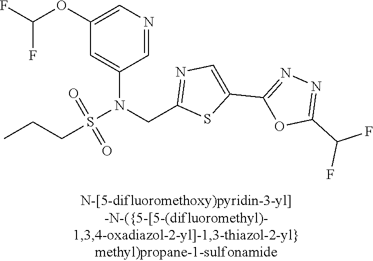 [SMILES: CCCS(=O)(=O)N(Cc1ncc(-c2nnc(C(F)F)o2)s1)c1cncc(OC(F)F)c1] | 1H NMR (400 MHz, CDCl3) δ 8.60 (s, 1 H), 8.47 (s, 1 H), 8.40 (s, 1 H), 7.67 (s, 1 H), 6.91 (t, J = 51.6 Hz, 1 H), 6.59 (t, J = 72.0 Hz, 1 H), 5.29 (s, 2 H), 3.19- 3.11 (m, 2 H), 1.99-1.86 (m, 2 H), 1.09 (t, J = 7.2 Hz, 3 H). LCMS RT = 1.794 min, m/z = 481.9 | 0.0144 |
| I-163 | 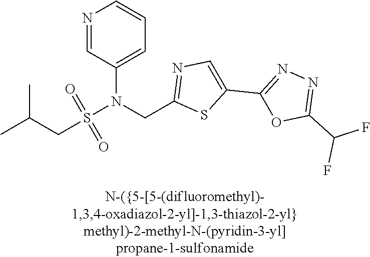 [SMILES: CC(C)CS(=O)(=O)N(Cc1ncc(-c2nnc(C(F)F)o2)s1)c1cccnc1] | 1H NMR (400 MHz, CDCl3) δ 8.70 (d, J = 2.4 Hz, 1 H), 8.60- 8.55 (m, 1 H), 8.41 (s, 1 H), 8.07- 8.01 (m, 1 H), 7.53-7.47 (m, 1 H) , 7.20 (t, J = 51.6 Hz, 1 H), 5.37 (s, 2 H), 3.20 (d, J = 6.4 Hz, 2 H), 2.34-2.22 (m, 1 H), 1.10 (d, J = 6.4 Hz, 6 H). LCMS RT = 1.423 min, m/z = 430.2 | 0.0161 |
| I-164 | 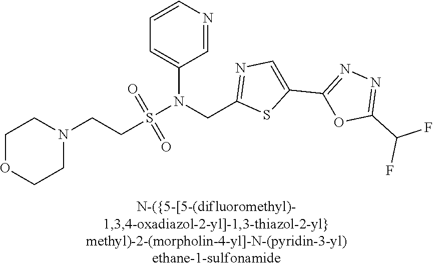 [SMILES: O=S(=O)(CCN1CCOCC1)N(Cc1ncc(-c2nnc(C(F)F)o2)s1)c1cccnc1] | 1H NMR (400 MHz, CDCl3) δ ppm 8.73 (d, J = 2.4 Hz, 1 H), 8.60-8.55 (m, 1 H), 8.38 (s, 1 H), 7.87-7.81 (m, 1 H), 7.38- 7.33 (m, 1 H), 6.91 (t, J = 51.6 Hz, 1 H), 5.32 (s, 2 H), 3.76- 3.72 (m, 4 H), 3.37 (t, J = 7.2 Hz, 2 H), 2.90 (t, J = 6.8 Hz, 2 H), 2.52-2.50 (m, 4 H). LCMS RT = 0.430 min, m/z = 487.2 | 0.0173 |
| I-165 | 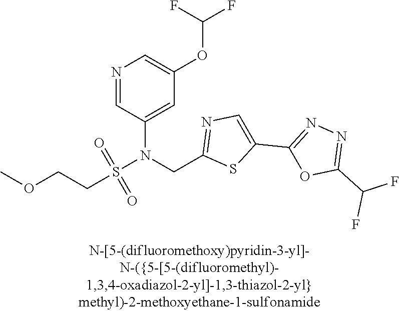 [SMILES: COCCS(=O)(=O)N(Cc1ncc(-c2nnc(C(F)F)o2)s1)c1cncc(OC(F)F)c1] | 1H NMR (400 MHz, CD3OD) δ 8.59 (s, 1 H), 8.40 (s, 2 H), 7.87 (d, J = 2.4 Hz, 1 H), 7.19 (t, J = 51.6 Hz, 1 H), 6.97 (t, J = 72.8 Hz, 1 H), 5.34 (s, 2 H), 3.83 (t, J = 5.2 Hz, 2 H), 3.52 (t, J = 5.6 Hz, 2 H), 3.30 (t, J = 1.6 Hz, 3 H). LCMS RT = 1.533 min, m/z = 498.2 | 0.0196 |
| I-166 | 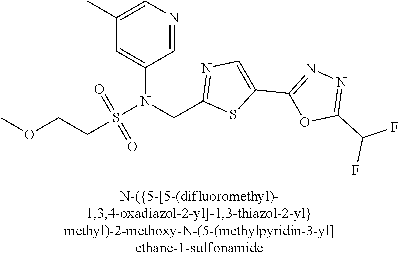 [SMILES: COCCS(=O)(=O)N(Cc1ncc(-c2nnc(C(F)F)o2)s1)c1cncc(C)c1] | 1H NMR (400 MHz, CD3OD) δ 8.49 (s, 1 H), 8.39 (s, 1 H), 8.34 (s, 1 H), 7.87 (s, 1 H), 7.20 (t, J = 51.6 Hz, 1 H), 5.31 (s, 2 H), 3.83 (t, J = 5.2 Hz, 2 H), 3.49 (t, J = 5.6 Hz, 2 H), 3.43 (s, 3 H), 2.38 (s, 3 H). LCMS RT = 2.329 min, m/z = 446.5 | 0.0266 |
| I-167 | 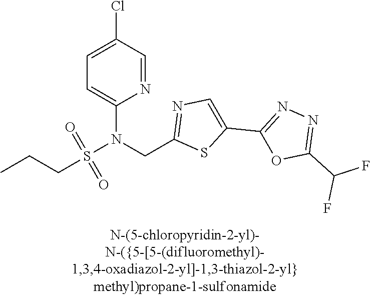 [SMILES: CCCS(=O)(=O)N(Cc1ncc(-c2nnc(C(F)F)o2)s1)c1ccc(Cl)cn1] | 1H NMR (400 MHz, CDCl3) 8 8.40-8.35 (m, 2 H), 7.73-7.68 (m, 1 H), 7.66-7.60 (m, 1 H), 6.89 (t, J = 51.6 Hz, 1 H), 5.55 (s, 2 H), 3.26-3.17 (m, 2 H), 1.93- 1.81 (m, 2 H), 1.05 (t, J = 7.4 Hz, 3 H). LCMS RT = 2.072 min, m/z = 449.8 | 0.0266 |
| I-168 | 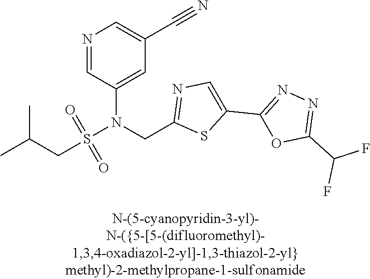 [SMILES: CC(C)CS(=O)(=O)N(Cc1ncc(-c2nnc(C(F)F)o2)s1)c1cncc(C#N)c1] | 1H NMR (400 MHz, CDCl3) δ 8.95 (d, J = 2.4 Hz, 1 H), 8.82 (s, 1 H), 8.42 (s, 1 H), 8.16 (s, 1 H), 6.92 (t, J = 52.0 Hz, 1 H), 5.28 (s, 2 H), 3.03 (d, J = 6.4 Hz, 2 H), 2.42-2.28 (m, 1 H), 1.12 (d, J = 6.8 Hz, 6 H). LCMS RT = 1.690 min, m/z = 455.2 | 0.0305 |
| I-169 | 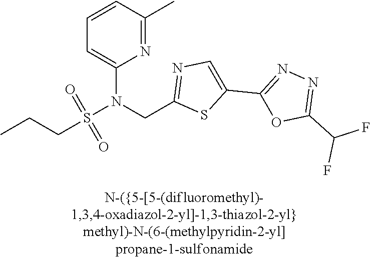 [SMILES: CCCS(=O)(=O)N(Cc1ncc(-c2nnc(C(F)F)o2)s1)c1cccc(C)n1] | 1H NMR (400 MHz, CDCl3) δ 8.38 (s, 1 H), 7.61 (t, J = 7.6 Hz, 1 H), 7.45 (d, J = 8.4 Hz, 1 H), 7.02-6.75 (m, 2 H), 5.59 (s, 2 H), 3.22-3.15 (m, 2 H), 2.52 (s, 3 H), 1.93-1.80 (m, 2 H), 1.03 (t, J = 7.6 Hz, 3 H). LCMS RT = 1.942 min, m/z = 429.9 | 0.0309 |
| I-170 | 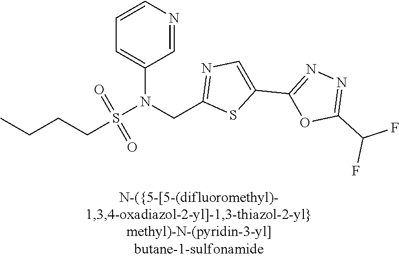 [SMILES: CCCCS(=O)(=O)N(Cc1ncc(-c2nnc(C(F)F)o2)s1)c1cccnc1] | 1H NMR (400 MHz, CDCl3) δ 8.70 (s, 1 H), 8.58 (d, J = 4.8 Hz, 1 H), 8.38 (s, 1 H), 7.90-7.80 (m, 1 H), 7.45-7.31 (m, 1 H), 6.91 (t, J = 51.2 Hz, 1 H), 5.29 (s, 2 H), 3.21-3.06 (m, 2 H), 1.92- 1.83 (m, 2 H), 1.52-1.43 (m, 2 H), 0.96 (t, J = 7.2 Hz, 3 H). LCMS RT = 0.623 min, m/z = 429.9 | 0.0264 |
| I-171 | 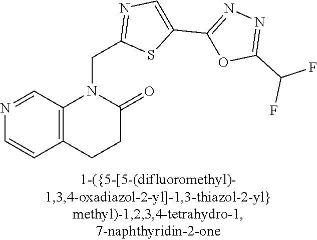 [SMILES: O=C1CCc2ccncc2N1Cc1ncc(-c2nnc(C(F)F)o2)s1] | 1H NMR (400 MHz, CDCl3) δ 8.55 (s, 1 H), 8.42 (s, 1 H), 8.31 (d, J = 3.6 Hz, 1 H), 7.16 (d, J = 4.4 Hz, 1 H), 6.89 (t, J = 51.6 Hz, 1 H), 5.52 (s, 2 H), 3.04 (t, J = 6.8 Hz, 2 H), 2.81 (t, J = 8.0 Hz, 2 H). LCMS RT = 1.398 min, m/z = 363.9 | 0.0215 |
| I-172 | 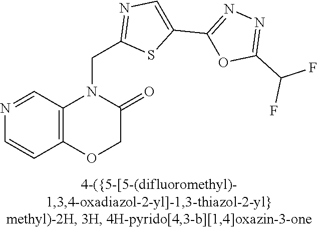 [SMILES: O=C1COc2ccncc2N1Cc1ncc(-c2nnc(C(F)F)o2)s1] | 1H NMR (400 MHz, CDCl3) δ 8.52 (s, 1 H), 8.43 (s, 1 H), 8.27 (d, J = 5.6 Hz, 1 H), 6.96 (d, J = 5.2 Hz, 1 H), 6.90 (t, J = 51.6 Hz, 1 H), 5.52 (s, 2 H), 4.85 (s, 2 H). LCMS RT = 0.418 min, m/z = 366.0 | 0.025 |
| I-173 | 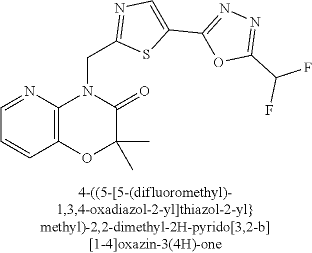 [SMILES: CC1(C)Oc2cccnc2N(Cc2ncc(-c3nnc(C(F)F)o3)s2)C1=O] | ¹H NMR (400 MHz, CDCl₃) δ 8.39 (s, 1 H), 8.01-8.05 (m, 1 H), 7.29 (dd, J = 8.1, 1.5 Hz, 1 H), 7.01 (dd, J = 8.1, 1.5 Hz, 1H), 6.89 (t, J = 51.6 Hz, 1 H), 5.70 (s, 2 H) LCMS: RT= 5.00 min, m/z = 394.0 | 0.108 |
| I-174 | 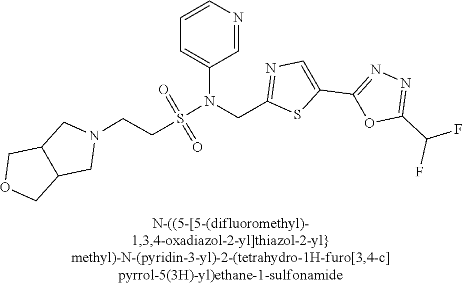 [SMILES: O=S(=O)(CCN1CC2COCC2C1)N(Cc1ncc(-c2nnc(C(F)F)o2)s1)c1cccnc1] | 1H NMR (400 MHz, CDCl3) δ 8.76 (s, 1 H), 8.58 (d, J = 4.4 Hz, 1 H), 8.39 (s, 1 H), 7.89 (d, J = 8.4 Hz, 1 H), 7.40-7.35 (m, 1 H), 6.91 (t, J = 51.6 Hz, 1 H), 5.33 (s, 2 H), 3.75-3.70 (m, 4 H), 3.49 (t, J = 6.8 Hz, 2 H), 3.07 (t, J = 6.8 Hz, 2 H), 2.99-2.96 (m, 4 H), 2.49 (d, J = 6.0 Hz, 2 H). LCMS RT = 1.435 min, m/z = 513.0 | 0.022 |
| I-175 | 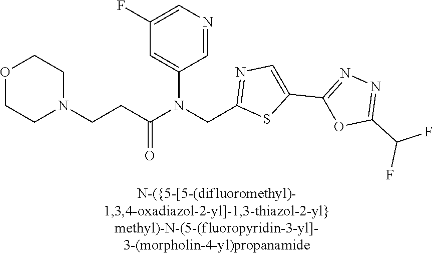 [SMILES: O=C(CCN1CCOCC1)N(Cc1ncc(-c2nnc(C(F)F)o2)s1)c1cncc(F)c1] | 1H NMR (400 MHz, CDCl3) δ 8.55 (d, J = 2.0 Hz, 1 H), 8.43- 8.36 (m, 2 H), 7.50-7.44 (m, 1 H), 6.92 (t, J = 51.6 Hz, 1 H), 5.20 (s, 2 H), 3.71-3.66 (m, 4 FH), 2.81 (t, J = 6.8 Hz, 2 H), 2.50- 2.22 (m, 6 H). LCMS RT = 0.400 min, m/z = 469.2 | 0.496 |
| I-176 | 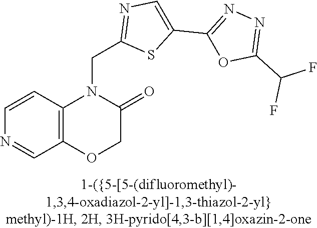 [SMILES: O=C1COc2cnccc2N1Cc1ncc(-c2nnc(C(F)F)o2)s1] | 1H NMR (400 MHz, CD3OD) δ 8.54-8.53 (m, 1 H), 8.13-8.10 (m, 1 H), 7.22 (t, J = 51.6 Hz, 1 H), 6.97-6.89 (m, 1 H), 5.84- 5.78 (m, 1 H), 4.72-4.45 (m, 4 H). LCMS RT = 1.091 min, m/z = 365.8 | 2.52 |
| I-177 | 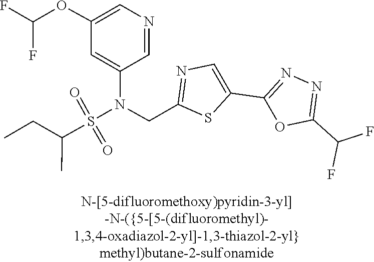 [SMILES: CCC(C)S(=O)(=O)N(Cc1ncc(-c2nnc(C(F)F)o2)s1)c1cncc(OC(F)F)c1] | 1H NMR (400 MHz, CDCl3) δ 8.59 (d, J = 2.0 Hz, 1 H), 8.45 (d, J = 2.4 Hz, 1 H), 8.37 (s, 1 H), 7.67 (t, J = 2.4 Hz, 1 H), 6.91 (t, J = 51.6 Hz, 1 H), 6.58 (t, J = 72.0 Hz, 1 H), 5.38-5.20 (m, 2 H), 3.15-3.07 (m, 1 H), 2.13-2.02 (m, 1 H), 1.73-1.60 (m, 1 H), 1.43 (d, J = 6.8 Hz, 3 H), 1.05 (t, J = 7.2 Hz, 3 H). LCMS RT = 1.580 min, m/z = 496.2 | 0.006 |
| I-178 | 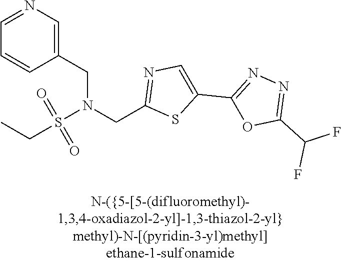 [SMILES: CCS(=O)(=O)N(Cc1cccnc1)Cc1ncc(-c2nnc(C(F)F)o2)s1] | 1H NMR (400 MHz, CDCl3) δ 8.62-8.55 (m, 2 H), 8.42 (s, 1 H), 7.88-7.82 (m, 1 H), 7.38- 7.34 (m, 1 H), 6.88 (t, J = 51.6 Hz, 1 H), 4.71 (s, 2 H), 4.60 (s, 2 H), 3.19 (q, J = 7.6 Hz, 2 H), 1.44 (t, J = 7.2 Hz, 3 H). LCMS RT = 1.031 min, m/z = 416.2 | 4.35 |
| I-179 | 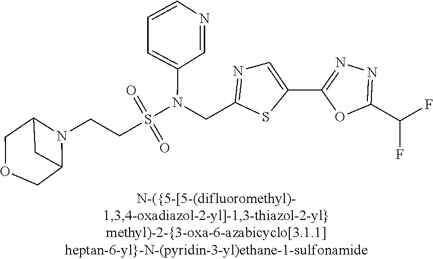 [SMILES: O=S(=O)(CCN1C2COCC1C2)N(Cc1ncc(-c2nnc(C(F)F)o2)s1)c1cccnc1] | 1H NMR (400 MHz, CDCl3) δ 8.76 (d, J = 2.4 Hz, 1 H), 8.58 (d, J = 3.2 Hz, 1 H), 8.38 (s, 1 H), 7.89-7.86 (m, 1 H), 7.39-7.35 (m, 1 H), 6.91 (t, J = 51.6 Hz, 1 H), 5.34 (s, 2 H), 4.22 (d, J = 11.2 Hz, 2 H), 3.81 (d, J = 11.6 Hz, 2 H), 3.64 (d, J = 6.4 Hz, 2 H), 3.36- 3.20 (m, 4 H), 2.74-2.69 (m, 1 H), 1.91 (d, J = 8.8 Hz, 1 H). LCMS RT = 1.438 min, m/z = 499.0 | 0.013 |
| I-180 | 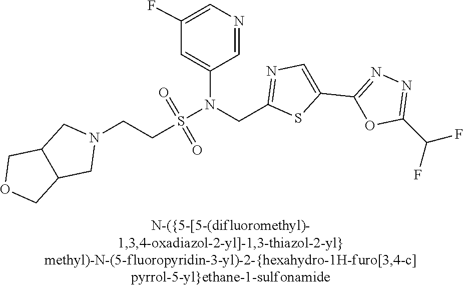 [SMILES: O=S(=O)(CCN1CC2COCC2C1)N(Cc1ncc(-c2nnc(C(F)F)o2)s1)c1cncc(F)c1] | 1H NMR (400 MHz, CDCl3) δ 8.62 (s, 1 H), 8.45 (d, J = 2.4 Hz, 1 H), 8.38 (s, 1 H), 7.75-7.70 (m, 1 H), 6.91 (t, J = 51.6 Hz, 1 H), 5.35 (s, 2 H), 3.78-3.69 (m, 4 H), 3.34 (t, J = 6.8 Hz, 2 H), 2.94 (t, J = 6.4 Hz, 2 H), 2.87- 2.86 (m, 2 H), 2.72-2.68 (m, 2 H), 2.49-2.46 (m, 2 H). LCMS RT = 0.417 min, m/z = 531.2 | 0.010 |
| I-181 | 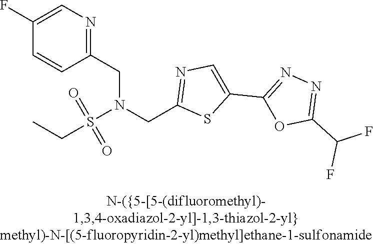 [SMILES: CCS(=O)(=O)N(Cc1ccc(F)cn1)Cc1ncc(-c2nnc(C(F)F)o2)s1] | 1H NMR (400 MHz, CDCl3) δ 8.44 (t, J = 2.0 Hz, 1 H), 8.41 (s, 1 H), 7.44 (d, J = 2.4 Hz, 1 H), 7.42 (s, 1 H), 6.92 (t, J = 51.6 Hz, 1 H), 4.86 (s, 2 H), 4.65 (s, 2 H), 3.23 (q, J = 7.2 Hz, 2 H), 1.42 (t, J = 7.2 Hz, 3 H). LCMS RT = 1.338 min, m/z = 434.2 | 3.46 |
| I-182 | 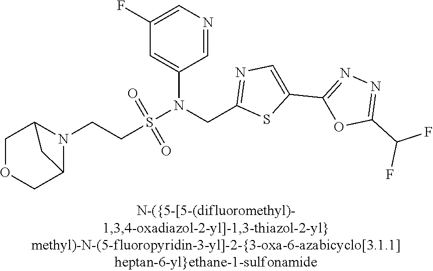 [SMILES: O=S(=O)(CCN1C2COCC1C2)N(Cc1ncc(-c2nnc(C(F)F)o2)s1)c1cncc(F)c1] | 1H NMR (400 MHz, CDCl3) δ 8.61 (s, 1 H), 8.46 (d, J = 2.4 Hz, 1 H), 8.39 (s, 1 H), 7.72-7.65 (m, 1 H), 6.91 (t, J = 51.6 Hz, 1 H), 5.35 (s, 2 H), 4.19 (d, J = 11.2 Hz, 2 H), 3.78 (d, J = 11.2 Hz, 2 H), 3.56 (d, J = 6.4 Hz, 2 H), 3.29- 3.25 (m, 2 H), 3.21-3.18 (m, 2 H), 2.70-2.65 (m, 1 H), 1.89 (d, J = 8.4 Hz, 1 H). LCMS RT = 0.844 min, m/z = 517.3 | 0.018 |
| I-183 | 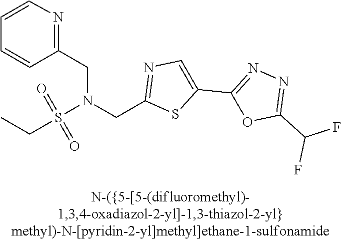 [SMILES: CCS(=O)(=O)N(Cc1ccccn1)Cc1ncc(-c2nnc(C(F)F)o2)s1] | 1H NMR (400 MHz, CDCl3) δ 8.59 (d, J = 4.4 Hz, 1 H), 8.40 (s, 1 H), 7.76-7.69 (m, 1 H), 7.39 (d, J = 7.6 Hz, 1 H), 7.25 (s, 1 H), 6.92 (t, J = 51.6 Hz, 1 H), 4.89 (s, 2 H), 4.68 (s, 2 H), 3.25 (q, J = 7.2 Hz, 2 H), 1.42 (t, J = 7.2 Hz, 3 H). LCMS RT = 0.932 min, m/z = 416.2 | 2.54 |
| I-184 | 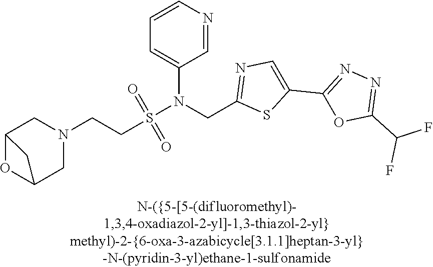 [SMILES: O=S(=O)(CCN1CC2CC(C1)O2)N(Cc1ncc(-c2nnc(C(F)F)o2)s1)c1cccnc1] | 1H NMR (400 MHz, CDCl3) δ 8.73 (d, J = 2.4 Hz, 1 H), 8.60 (d, J = 3.2 Hz, 1 H), 8.39 (s, 1 H), 7.89-7.82 (m, 1 H), 7.40-7.33 (m, 1 H), 6.91 (t, J = 51.6 Hz, 1 H), 5.31 (s, 2 H), 4.53 (d, J = 6.4 Hz, 2 H), 3.46-3.42 (m, 2 H), 3.21-3.02 (m, 5 H), 2.86 (d, J = 11.2 Hz, 2 H), 2.29 (d, J = 8.4 Hz, 1 H). LCMS RT = 1.352 min, m/z = 498.9 | 0.014 |
| I-185 | 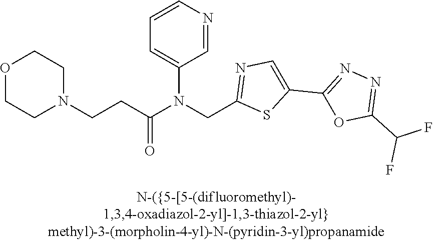 [SMILES: O=C(CCN1CCOCC1)N(Cc1ncc(-c2nnc(C(F)F)o2)s1)c1cccnc1] | 1H NMR (400 MHz, CDCl3) δ 8.65 (d, J = 4.4 Hz, 1 H), 8.55 (d, J = 2.4 Hz, 1 H), 8.38 (s, 1 H), 7.63 (d, J = 8.0 Hz, 1 H), 7.43- 7.39 (m, 1 H), 6.92 (t, J = 51.6 Hz, 1 H), 5.21 (s, 2 H), 3.65 (t, J = 4.4 Hz, 4 H), 2.73 (t, J = 7.2 Hz, 2 H), 2.38-2.33 (m, 6 H). LCMS RT = 0.411 min, m/z = 451.2 | 0.516 |
| I-186 | 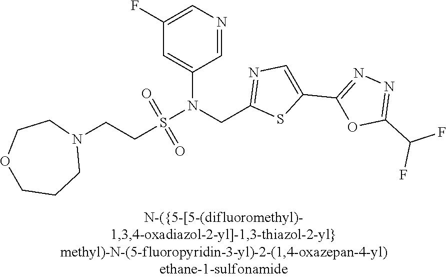 [SMILES: O=S(=O)(CCN1CCCOCC1)N(Cc1ncc(-c2nnc(C(F)F)o2)s1)c1cncc(F)c1] | 1H NMR (400 MHz, CDCl3) δ 8.58 (s, 1 H), 8.47 (d, J = 2.4 Hz, 1 H), 8.40 (s, 1 H), 7.70-7.67 (m, 1 H), 6.91 (t, J = 51.6 Hz, 1 H), 5.32 (s, 2 H), 3.81 (t, J = 6.4 Hz, 2 H), 3.75 (t, J = 4.8 Hz, 2 H), 3.41 (t, J = 7.2 Hz, 2 H), 3.12 (t, J = 7.6 Hz, 2 H), 2.84-2.76 (m, 4 H), 1.97-1.92 (m, 2 H). LCMS RT = 0.414 min, m/z = 519.2 | 0.0276 |
| I-187 | 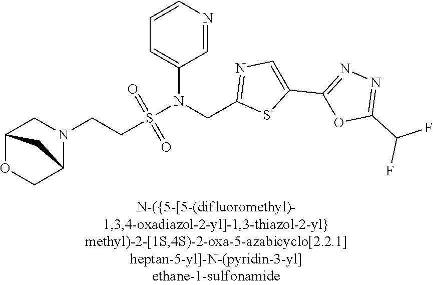 [SMILES: O=S(=O)(CCN1C[C@@H]2C[C@H]1CO2)N(Cc1ncc(-c2nnc(C(F)F)o2)s1)c1cccnc1] | 1H NMR (400 MHz, CDCl3) δ 8.74 (d, J = 2.4 Hz, 1 H), 8.60- 8.57 (m, 1 H), 8.38 (s, 1 H), 7.88- 7.85 (m, 1 H), 7.40-7.26 (m, 1 H), 6.91 (t, J = 51.6 Hz, 1 H), 5.37-5.25 (m, 2 H), 4.47 (s, 1 H), 4.02 (d, J = 8.4 Hz, 1 H), 3.70- 3.55 (m, 1 H), 3.58 (s, 1 H), 3.36 (t, J = 6.8 Hz, 2 H), 3.22- 3.09 (m, 2 H), 3.00-2.92 (m, 1 H), 2.60 (d, J = 9.6 Hz, 1 H), 1.86- 1.80 (m, 2 H). LCMS RT = 0.429 min, m/z = 499.2 | 0.0247 |
| I-188 | 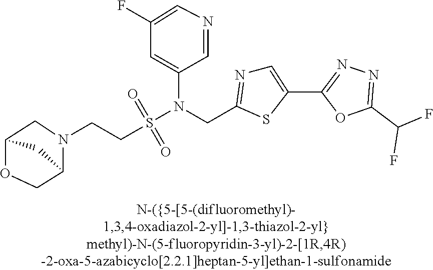 [SMILES: O=S(=O)(CCN1C[C@H]2C[C@@H]1CO2)N(Cc1ncc(-c2nnc(C(F)F)o2)s1)c1cncc(F)c1] | 1H NMR (400 MHz, CDCl3) δ 8.58 (s, 1 H), 8.47 (d, J = 2.4 Hz, 1 H), 8.39 (s, 1 H), 7.69-7.66 (m, 1 H), 6.91 (t, J = 51.6 Hz, 1 H), 5.37-5.27 (m, 2 H), 4.47 (s, 1 H), 4.02 (d, J = 8.0 Hz, 1 H), 3.68 (dd, J = 8.4, 1.6 Hz, 1 H), 3.58 (s, 1 H), 3.38 (t, J = 6.8 Hz, 2 H), 3.22-3.09 (m, 2 H), 2.98- 2.96 (m, 1 H), 2.60 (d, J = 10.0 Hz, 1 H), 1.85-1.83 (m, 2 H). LCMS RT = 0.457 min, m/z = 517.2 | 0.0151 |
| I-189 | 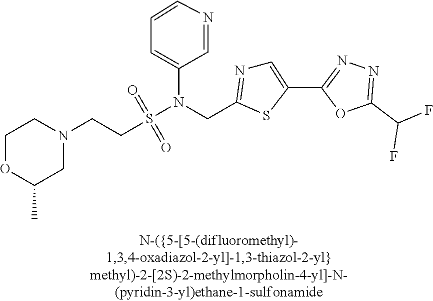 [SMILES: C[C@H]1CN(CCS(=O)(=O)N(Cc2ncc(-c3nnc(C(F)F)o3)s2)c2cccnc2)CCO1] | 1H NMR (400 MHz, CDCl3) δ 8.73 (d, J = 2.4 Hz, 1 H), 8.59 (d, J = 3.6 Hz, 1 H), 8.38 (s, 1 H), 7.88-7.85 (m, 1 H), 7.39-7.35 (m, 1 H), 6.91 (t, J = 51.6 Hz, 1 H), 5.32 (s, 2 H), 3.91-3.88 (m, 1 H), 3.71-3.62 (m, 2 H), 3.38 (t, J = 6.8 Hz, 2 H), 2.90 (t, J = 7.2 Hz, 2 H), 2.76-2.69 (m, 2 H), 2.28-2.21 (m, 1 H), 1.93 (t, J = 10.8 Hz, 1 H), 1.17 (d, J = 6.0 Hz, 3 H). LCMS RT = 1.427 min, m/z = 500.9 | 0.0363 |
| I-190 | 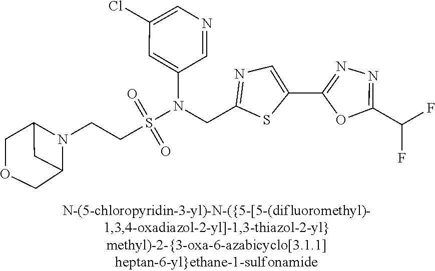 [SMILES: O=S(=O)(CCN1C2COCC1C2)N(Cc1ncc(-c2nnc(C(F)F)o2)s1)c1cncc(Cl)c1] | 1H NMR (400 MHz, CDCl3) δ 8.65 (d, J = 2.4 Hz, 1 H), 8.54 (d, J = 2.4 Hz, 1 H), 8.39 (s, 1 H), 7.95 (t, J = 2.4 Hz, 1 H), 6.91 (t, J = 51.6 Hz, 1 H), 5.34 (s, 2 H), 4.21 (d, J = 11.6 Hz, 2 H), 3.80 (d, J = 11.2 Hz, 2 H), 3.60 (d, J = 6.0 Hz, 2 H), 3.32-3.25 (m, 2 H), 3.24-3.17 (m, 2 H), 2.77- 2.63 (m, 1 H), 1.91 (d, J = 8.4 Hz, 1 H). LCMS RT = 0.968 min, m/z = 533.2 | 0.016 |
| I-191 | 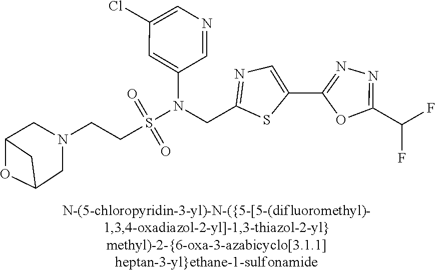 [SMILES: O=S(=O)(CCN1CC2CC(C1)O2)N(Cc1ncc(-c2nnc(C(F)F)o2)s1)c1cncc(Cl)c1] | 1H NMR (400 MHz, CDCl3) δ 8.62 (d, J = 2.0 Hz, 1 H), 8.55 (d, J = 2.0 Hz, 1 H), 8.41 (s, 1 H), 7.90 (t, J = 2.4 Hz, 1 H), 6.91 (t, J = 51.2 Hz, 1 H), 5.29 (s, 2 H), 4.52 (d, J = 6.4 Hz, 2 H), 3.43 (t, J = 6.8 Hz, 2 H), 3.18 (t, J = 7.2 Hz, 2 H), 3.10 (d, J = 10.8 Hz, 2 H), 3.06-2.80 (m, 3 H), 2.28 (d, J = 8.0 Hz, 1 H). LCMS RT = 0.482 min, m/z = 533.2 | 0.0124 |
| I-192 | 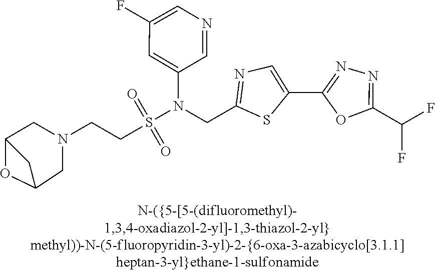 [SMILES: O=S(=O)(CCN1CC2CC(C1)O2)N(Cc1ncc(-c2nnc(C(F)F)o2)s1)c1cncc(F)c1] | 1H NMR (400 MHz, CDCl3) δ 8.57 (s, 1 H), 8.46 (d, J = 2.4 Hz, 1 H), 8.40 (s, 1 H), 7.73-7.61 (m, 1 H), 6.92 (t, J = 51.2 Hz, 1 H), 5.31 (s, 2 H), 4.52 (d, J = 6.0 Hz, 2 H), 3.46 (t, J = 6.8 Hz, 2 H), 3.18 (t, J = 7.6 Hz, 2 H), 3.12 (d, J = 11.0 Hz, 2 H), 3.08-3.01 (m, 1 H), 2.86 (d, J = 11.2 Hz, 2 H), 2.27 (d, J = 8.0 Hz, 1 H). LCMS RT = 1.368 min, m/z = 517.2 | 0.0168 |
| I-193 | 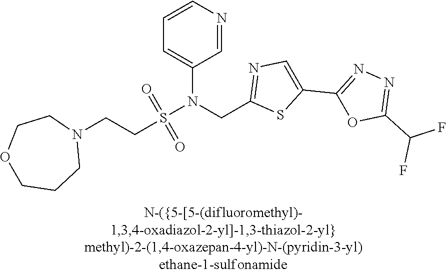 [SMILES: O=S(=O)(CCN1CCCOCC1)N(Cc1ncc(-c2nnc(C(F)F)o2)s1)c1cccnc1] | 1H NMR (400 MHz, CDCl3) δ ppm 8.73 (d, J = 2.4 Hz, 1 H), 8.58 (d, J = 4.0 Hz, 1 H), 8.38 (s, 1 H), 7.86 (d, J = 8.0 Hz, 1 H), 7.39-7.35 (m, 1 H), 6.92 (t, J = 51.6 Hz, 1 H), 5.32 (s, 2 H), 3.80 (t, J = 6.0 Hz, 2 H), 3.75-3.73 (m, 2 H), 3.37 (t, J = 7.6 Hz, 2 H), 3.10 (t, J = 6.4 Hz, 2 H), 2.79- 2.75 (m, 4 H), 1.95-1.91 (m, 2 H). LCMS RT = 0.429 min, m/z = 501.2 | 0.0203 |
| I-194 | 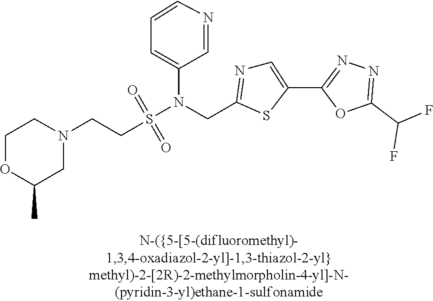 [SMILES: C[C@@H]1CN(CCS(=O)(=O)N(Cc2ncc(-c3nnc(C(F)F)o3)s2)c2cccnc2)CCO1] | 1H NMR (400 MHz, CDCl3) δ ppm 8.73 (s, 1 H), 8.59 (d, J = 4.4 Hz, 1 H), 8.38 (s, 1 H), 7.86- 7.83 (m, 1 H), 7.38-7.35 (m, 1 H), 6.91 (t, J = 51.2 Hz, 1 H), 5.31 (s, 2 H), 3.87-3.67 (m, 1 H), 3.66-3.64 (m, 2 H), 3.64 (t, J = 6.8 Hz, 2 H), 2.89 (t, J = 6.8 Hz, 2 H), 2.74-2.67 (m, 2 H), 2.24-2.23 (m, 1 H), 1.92 (t, J = 6.8 Hz, 1 H), 1.17 (d, J = 6.8 Hz, 3 H). LCMS RT = 0.748 min, m/z = 501.2 | 0.0267 |
| I-195 | 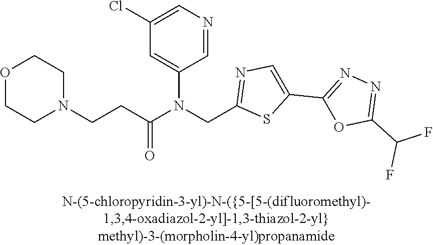 [SMILES: O=C(CCN1CCOCC1)N(Cc1ncc(-c2nnc(C(F)F)o2)s1)c1cncc(Cl)c1] | 1H NMR (400 MHz, CDCl3) δ ppm 8.62 (s, 1 H), 8.44 (s, 1 H), 8.39 (s, 1 H), 7.72 (s, 1 H), 6.92 (t, J = 51.6 Hz, 1 H), 5.18 (s, 2 H), 3.17-3.69 (m, 8 H), 2.86- 2.83 (m, 2 H), 2.44-2.41 (m, 2 H). LCMS RT = 1.381 min, m/z = 485.3 | 0.312 |
| I-196 | 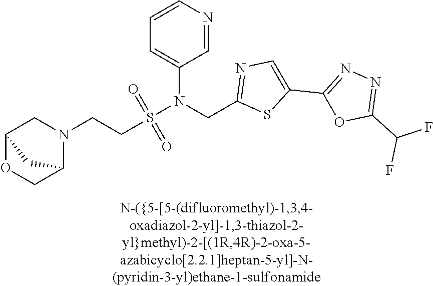 [SMILES: O=S(=O)(CCN1C[C@H]2C[C@@H]1CO2)N(Cc1ncc(-c2nnc(C(F)F)o2)s1)c1cccnc1] | 1H NMR (400 MHz, CDCl3) δ 8.74 (s, 1 H), 8.59 (d, J = 4.0 Hz, 1 H), 8.39 (s, 1 H), 7.87 (d, J = 8.4 Hz, 1 H), 7.39-7.35 (m, 1 H), 6.91 (t, J = 51.6 Hz, 1 H), 5.36-5.27 (m, 2 H), 4.48 (s, 1 H), 4.05 (d, J = 8.4 Hz, 1 H), 3.70- 3.66 (m, 2 H), 3.43 (t, J = 6.8 Hz, 2 H), 3.26-3.13 (m, 2 H), 3.03 (d, J = 10.0 Hz, 1 H), 2.66 (d, J = 10.4 Hz, 1 H), 1.95-1.85 (m, 2 H). LCMS RT = 1.117 min, m/z = 499.2 | 0.0497 |
| I-197 | 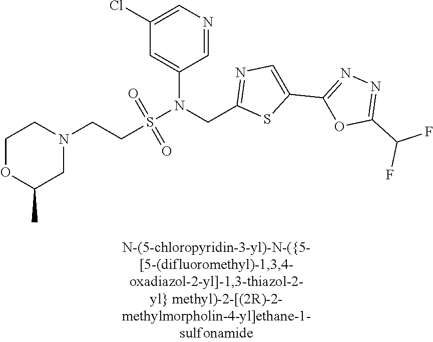 [SMILES: C[C@@H]1CN(CCS(=O)(=O)N(Cc2ncc(-c3nnc(C(F)F)o3)s2)c2cncc(Cl)c2)CCO1] | 1H NMR (400 MHz, CDCl3) δ 8.63 (d, J = 2.0 Hz, 1 H), 8.54 (d, J = 2.0 Hz, 1 H), 8.39 (s, 1 H), 7.91 (t, J = 2.0 Hz, 1 H), 6.91 (t, J = 51.6 Hz, 1 H), 5.30 (s, 2 H), 3.93-3.85 (m, 1 H), 3.72-3.60 (m, 2 H), 3.36 (t, J = 6.8 Hz, 2 H), 2.88 (t, J = 6.8 Hz, 2 H), 2.75- 2.65 (m, 2 H), 2.28-2.17 (m, 1 H), 1.91 (t, J = 10.8 Hz, 1 H), 1.17 (d, J = 6.4 Hz, 3 H). LCMS RT = 1.558 min, m/z = 535.2 | 0.0472 |
| I-198 | 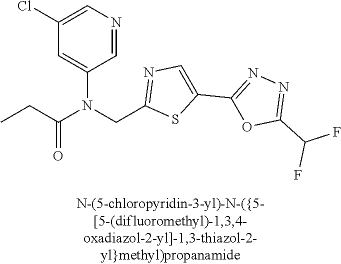 [SMILES: CCC(=O)N(Cc1ncc(-c2nnc(C(F)F)o2)s1)c1cncc(Cl)c1] | 1H NMR (400 MHz, CDCl3) δ 8.62 (s, 1 H), 8.44-8.35 (m, 2 H), 7.67 (t, J = 2.0 Hz, 1 H), 6.92 (t, J = 51.6 Hz, 1 H), 5.18 (s, 2 H), 2.18 (q, J = 7.2 Hz, 2 H), 1.13 (t, J = 7.6 Hz, 3 H). LCMS RT = 1.929 min, m/z = 400.2 | 0.299 |
| I-199 | 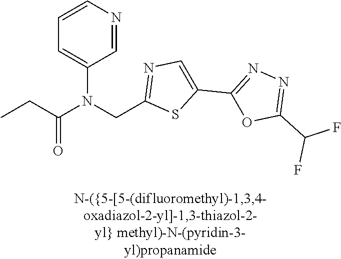 [SMILES: CCC(=O)N(Cc1ncc(-c2nnc(C(F)F)o2)s1)c1cccnc1] | 1H NMR (400 MHz, CDCl3) δ 8.64 (d, J = 4.0 Hz, 1 H), 8.53 (s, 1 H), 8.37 (s, 1 H), 7.61 (d, J = 8.0 Hz, 1 H), 7.42 - 7.36 (m, 1 H), 6.91 (t, J = 51.6 Hz, 1 H), 5.20 (s, 2 H), 2.15 (q, J = 7.2 Hz, 2 H), 1.12 (t, J = 7.2 Hz, 3 H). LCMS RT = 1.372 min, m/z = 366.2 | 0.355 |
| I-200 | 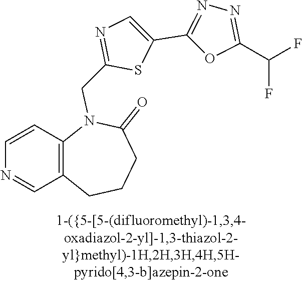 [SMILES: O=C1CCCc2cnccc2N1Cc1ncc(-c2nnc(C(F)F)o2)s1] | 1H NMR (400 MHz, CDCl3) δ 8.56 (d, J = 5.2 Hz, 1 H), 8.46 (s, 1 H), 8.39 (s, 1 H), 7.36 (d, J = 5.6 Hz, 1 H), 6.91 (t, J = 51.6 Hz, 1 H), 5.33 (s, 2 H), 2.80 (t, J = 7.2 Hz, 2 H), 2.44 (t, J = 7.2 Hz, 2 H), 2.36-2.24 (m, 2 H). LCMS RT = 0.993 min, m/z = 378.2 | 0.125 |
| I-201 | 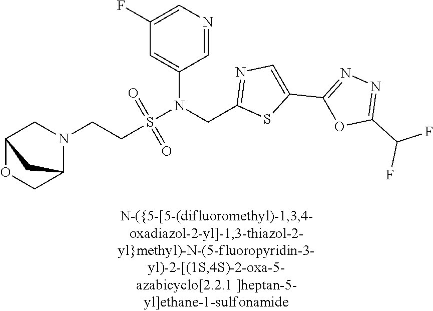 [SMILES: O=S(=O)(CCN1C[C@@H]2C[C@H]1CO2)N(Cc1ncc(-c2nnc(C(F)F)o2)s1)c1cncc(F)c1] | 1H NMR (400 MHz, CDCl3) δ 8.57 (s, 1 H), 8.46 (d, J = 2.4 Hz, 1 H), 8.39 (s, 1 H), 7.70-7.62 (m, 1 H), 6.91 (t, J = 51.6 Hz, 1 H), 5.39-5.25 (m, 2 H), 4.46 (s, 1 H), 4.00 (d, J = 8.0 Hz, 1 H), 3.69-3.64 (m, 1 H), 3.52 (s, 1 H), 3.34 (t, J = 6.8 Hz, 2 H), 3.19- 3.07 (m, 2 H), 2.94 (d, J = 10.0 Hz, 1 H), 2.56 (d, J = 10.0 Hz, 1 H), 1.89-1.79 (m, 2 H). LCMS RT = 0.459 min, m/z = 517.2 | 0.0207 |
| I-202 | 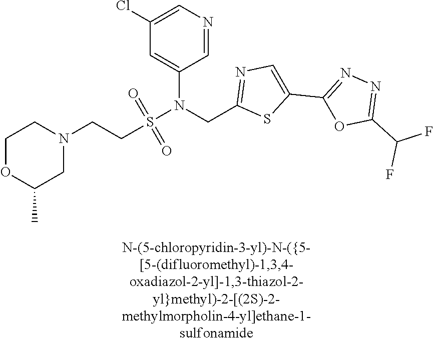 [SMILES: C[C@H]1CN(CCS(=O)(=O)N(Cc2ncc(-c3nnc(C(F)F)o3)s2)c2cncc(Cl)c2)CCO1] | 1H NMR (400 MHz, CDCl3) δ 8.63 (s, 1 H), 8.54 (s, 1 H), 8.39 (s, 1 H), 7.91 (s, 1 H), 6.91 (t, J = 51.2 Hz, 1 H), 5.30 (s, 2 H), 3.94- 3.85 (m, 1 H), 3.74-3.62 (m, 2 H), 3.43 (t, J = 6.8 Hz, 2 H), 2.88 (t, J = 6.8 Hz, 2 H), 2.72 (t, J = 12.8 Hz, 2 H), 2.28-2.17 (m, 1 H), 1.92 (t, J = 9.6 Hz, 1 H), 1.17 (d, J = 6.0 Hz, 3 H). LCMS RT = 1.561 min, m/z = 535.2 | 0.0127 |
| I-203 | 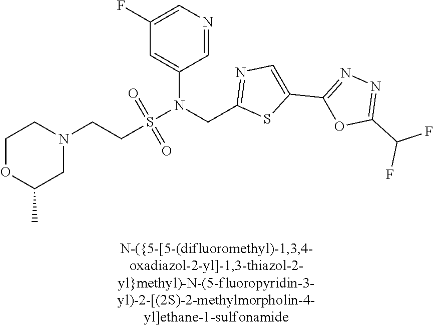 [SMILES: C[C@H]1CN(CCS(=O)(=O)N(Cc2ncc(-c3nnc(C(F)F)o3)s2)c2cncc(F)c2)CCO1] | 1H NMR (400 MHz, CDCl3) δ 8.58 (s, 1 H), 8.47 (d, J = 2.4 Hz, 1 H), 8.40 (s, 1 H), 7.68 (d, J = 9.2 Hz, 1 H), 6.92 (t, J = 51.2 Hz, 1 H), 5.32 (s, 2 H), 3.91-3.71 (m, 1 H), 3.72-3.64 (m, 2 H), 3.41 (t, J = 6.8 Hz, 2 H), 2.91 (t, J = 7.2 Hz, 2 H), 2.73 (t, J = 12.8 Hz, 2 H), 2.29-2.22 (m, 1 H), 1.96-1.94 (m, 1 H), 1.17 (d, J = 6.4 Hz, 3 H). LCMS RT = 1.660 min, m/z = 519.0 | 0.0254 |
| I-204 | 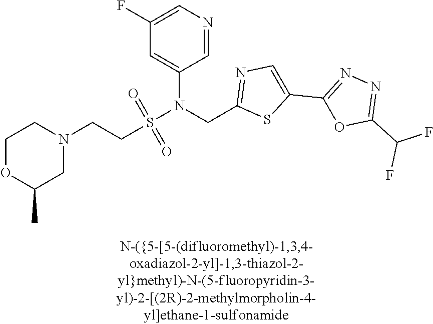 [SMILES: C[C@@H]1CN(CCS(=O)(=O)N(Cc2ncc(-c3nnc(C(F)F)o3)s2)c2cncc(F)c2)CCO1] | 1H NMR (400 MHz, CDCl3) δ 8.58 (s, 1 H), 8.47 (s, 1 H), 8.40 (s, 1 H), 7.70-7.66 (m, 1 H), 6.91 (t, J = 51.6 Hz, 1 H), 5.32 (s, 2 H), 3.89 (d, J = 9.6 Hz, 1 H), 3.70-3.60 (m, 2 H), 3.37 (t, J = 6.8 Hz, 2 H), 2.88 (t, J = 6.8 Hz, 2 H), 2.72-2.65 (m, 2 H), 2.26- 2.20 (m, 1 H), 1.91 (t, J = 10.4 Hz, 1 H), 1.17 (d, J = 6.0 Hz, 3 H). LCMS RT = 1.664 min, m/z = 518.9 | 0.0336 |
| I-205 | 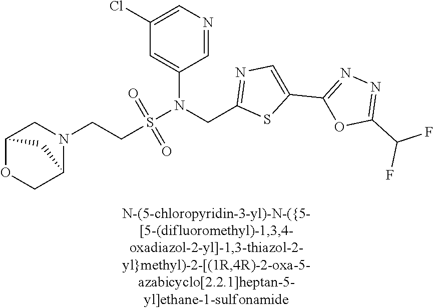 [SMILES: O=S(=O)(CCN1C[C@H]2C[C@@H]1CO2)N(Cc1ncc(-c2nnc(C(F)F)o2)s1)c1cncc(Cl)c1] | 1H NMR (400 MHz, CDCl3) δ 8.62 (d, J = 2.0 Hz, 1 H), 8.54 (d, J = 2.0 Hz, 1 H), 8.40 (s, 1 H), 7.91 (t, J = 2.0 Hz, 1 H), 6.91 (t, J = 51.6 Hz, 1 H), 5.40-5.21 (m, 2 H), 4.47 (s, 1 H), 4.02 (d, J = 8.0 Hz, 1 H), 3.68-3.63 (m, 1 H), 3.56 (s, 1 H), 3.35 (t, J = 6.8 Hz, 2 H), 3.25-3.05 (m, 2 H), 3.01- 2.90 (m, 1 H), 2.59 (d, J = 10.0 Hz, 1 H), 1.93-1.80 (m, 2 H). LCMS RT = 0.919 min, m/z = 533.2 | 0.0159 |
| I-206 | 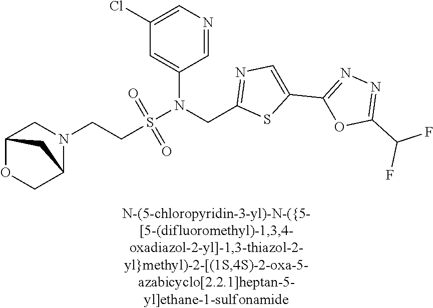 [SMILES: O=S(=O)(CCN1C[C@@H]2C[C@H]1CO2)N(Cc1ncc(-c2nnc(C(F)F)o2)s1)c1cncc(Cl)c1] | 1H NMR (400 MHz, CDCl3) δ 8.62 (d, J = 2.0 Hz, 1 H), 8.54 (d, J = 2.0 Hz, 1 H), 8.39 (s, 1 H), 7.91 (t, J = 2.0 Hz, 1 H), 6.91 (t, J = 51.6 Hz, 1 H), 5.37-5.25 (m, 2 H), 4.46 (s, 1 H), 4.01 (d, J = 8.4 Hz, 1 H), 3.69-3.62 (m, 1 H), 3.51 (s, 1 H), 3.31 (t, J = 6.8 Hz, 2 H), 3.18-3.07 (m, 2 H), 2.93 (d, J = 10.0 Hz, 1 H), 2.56 (d, J = 9.6 Hz, 1 H), 1.89-1.84 (q, J = 7.2 Hz, 2 H). LCMS RT = 0.472 min, m/z = 533.2 | 0.0162 |
| I-207 | 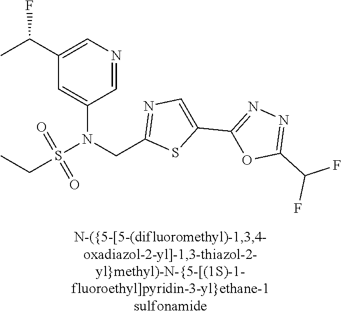 [SMILES: CCS(=O)(=O)N(Cc1ncc(-c2nnc(C(F)F)o2)s1)c1cncc([C@H](C)F)c1] | 1H NMR (400 MHz, CDCl3) δ 8.67 (d, J = 2.4 Hz, 1 H), 8.56 (s, 1 H), 8.39 (s, 1 H), 7.83 (s, 1 H), 6.91 (t, J = 51.6 Hz, 1 H), 5.79- 5.60 (m, 1 H), 5.30 (s, 2 H), 3.21 (q, J = 7.2 Hz, 2 H), 1.69-1.55 (m, 3 H), 1.46 (t, J = 7.6 Hz, 3 H). LCMS RT = 1.500 min, m/z = 447.8 | 0.0137 |
| I-208 | 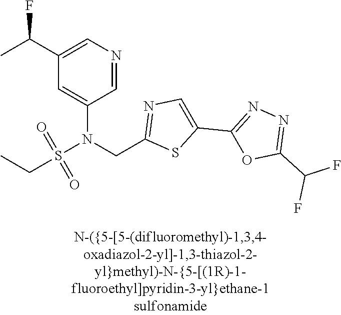 [SMILES: CCS(=O)(=O)N(Cc1ncc(-c2nnc(C(F)F)o2)s1)c1cncc([C@@H](C)F)c1] | 1H NMR (400 MHz, CDCl3) δ 8.67 (d, J = 2.0 Hz, 1 H), 8.56 (s, 1 H), 8.39 (s, 1 H), 7.83 (s, 1 H), 6.91 (t, J = 52.0 Hz, 1 H), 5.78- 5.62 (m, 1 H), 5.30 (s, 2 H), 3.21 (q, J = 7.2 Hz, 2 H), 1.74-1.62 (m, 3 H), 1.46 (t, J = 7.6 Hz, 3 H). LCMS RT = 1.503 min, m/z = 447.8 | 0.027 |
| I-209 | 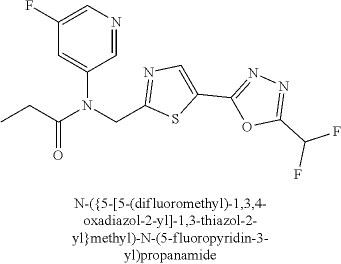 [SMILES: CCC(=O)N(Cc1ncc(-c2nnc(C(F)F)o2)s1)c1cncc(F)c1] | 1H NMR (400 MHz, CDCl3) δ 8.54 (d, J = 2.4 Hz, 1 H), 8.39- 8.35 (m, 2 H), 7.44-7.40 (m, 1 H), 6.92 (t, J = 51.6 Hz, 1 H), 5.19 (s, 2 H), 2.19 (q, J = 6.8 Hz, 2 H), 1.13 (t, J = 7.6 Hz, 3 H). LCMS RT = 1.755 min, m/z = 384.2 | 0.284 |
| I-210 | 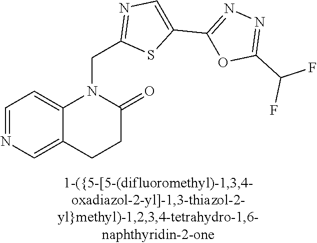 [SMILES: O=C1CCc2cnccc2N1Cc1ncc(-c2nnc(C(F)F)o2)s1] | 1H NMR (400 MHz, CDCl3) δ 8.49-8.37 (m, 3 H), 7.19 (d, J = 5.6 Hz, 1 H), 6.89 (t, J = 51.6 Hz, 1 H), 5.46 (s, 2 H), 3.10-3.01 (m, 2 H), 2.91-2.80 (m, 2 H). LCMS RT = 0.971 min, m/z = 364.2 | 0.0487 |
| I-211 | 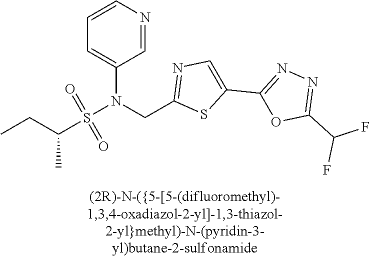 [SMILES: CC[C@@H](C)S(=O)(=O)N(Cc1ncc(-c2nnc(C(F)F)o2)s1)c1cccnc1] | 1H NMR (400 MHz, CDCl3) δ 8.70 (s, 1 H), 8.57-8.55 (m, 1 H), 8.35 (s, 1 H), 7.87-7.82 (m, 1 H), 7.38-7.32 (m, 1 H), 6.91 (t, J = 51.6 Hz, 1 H), 5.38-5.20 (m, 2 H), 3.14-3.06 (m, 1 H), 2.13-2.05 (m, 1 H), 1.72-1.65 (m, 1 H), 1.45 (d, J = 6.8 Hz, 3 H), 1.04 (t, J = 7.6 Hz, 3 H). LCMS RT = 0.563 min, m/z = 430.1 | 0.0137 |
| I-212 | 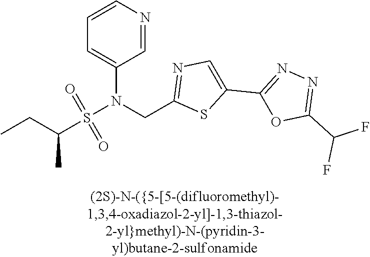 [SMILES: CC[C@H](C)S(=O)(=O)N(Cc1ncc(-c2nnc(C(F)F)o2)s1)c1cccnc1] | 1H NMR (400 MHz, CDCl3) δ 8.70 (s, 1 H), 8.58-8.55 (m, 1 H), 8.35 (s, 1 H), 7.89-7.86 (m, 1 H), 7.34 (q, J = 4.8 Hz, 1 H), 6.91 (t, J = 51.6 Hz, 1 H), 5.39- 5.23 (m, 2 H), 3.14-3.06 (m, 1 H), 2.14-2.04 (m, 1 H), 1.74- 1.65 (m, 1 H), 1.45 (d, J = 6.8 Hz, 3 H), 1.04 (t, J = 7.6 Hz, 3 H). LCMS RT = 0.563 min, m/z = 430.1 | 0.0105 |
| I-213 | 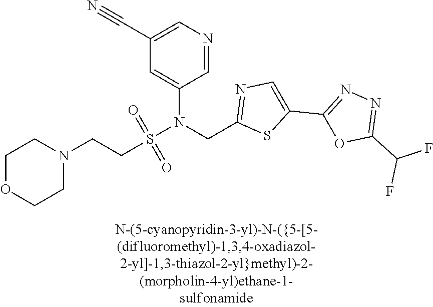 [SMILES: N#Cc1cncc(N(Cc2ncc(-c3nnc(C(F)F)o3)s2)S(=O)(=O)CCN2CCOCC2)c1] | 1H NMR (400 MHz, CDCl3) δ 8.96 (d, J = 2.4 Hz, 1 H), 8.82 (s, 1 H), 8.41 (s, 1 H), 8.19 (s, 1 H), 6.92 (t, J = 51.6 Hz, 1 H), 5.33 (s, 2 H), 3.74 (t, J = 4.8 Hz, 4 H), 3.38 (t, J = 6.8 Hz, 2 H), 2.89 (t, J = 7.2 Hz, 2 H), 2.50 (t, J = 4.8 Hz, 4 H). LCMS RT = 1.295 min, m/z = 512.2 | 0.0902 |
| I-214 | 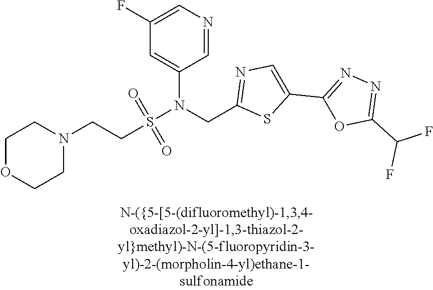 [SMILES: O=S(=O)(CCN1CCOCC1)N(Cc1ncc(-c2nnc(C(F)F)o2)s1)c1cncc(F)c1] | 1H NMR (400 MHz, CDCl3) δ ppm 8.59 (s, 1 H), 8.47 (d, J = 2.4 Hz, 1 H), 8.40 (s, 1 H), 7.71- 7.67 (m, 1 H), 6.93 (t, J = 51.6 Hz, 1 H), 5.33 (s, 2 H), 3.78- 3.72 (m, 4 H), 3.40 (t, J = 6.8 Hz, 2 H), 2.91 (t, J = 6.8 Hz, 2 H), 2.53-2.51 (m, 4 H). LCMS RT = 0.908 min, m/z = 505.2 | 0.0154 |
| I-215 | 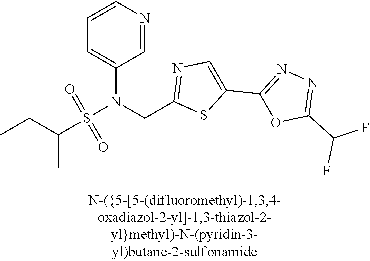 [SMILES: CCC(C)S(=O)(=O)N(Cc1ncc(-c2nnc(C(F)F)o2)s1)c1cccnc1] | 1H NMR (400 MHz, CDCl3) δ 8.72 (d, J = 2.8 Hz, 1 H), 8.58- 8.56 (m, 1 H), 8.35 (s, 1 H), 7.89- 7.86 (m, 1 H), 7.36 (q, J = 4.8 Hz, 1 H), 6.91 (t, J = 51.6 Hz, 1 H), 5.37-5.26 (m, 2 H), 3.14- 3.05 (m, 1 H), 2.12-2.06 (m, 1 H), 1.75-1.69 (m, 1 H), 1.45 (d, J = 6.8 Hz, 3 H), 1.04 (t, J = 7.6 Hz, 3 H). LCMS RT = 0.560 min, m/z = 430.1 | 0.00675 |
| I-216 | 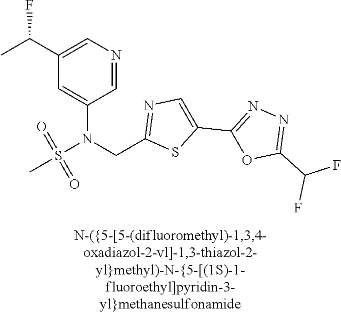 [SMILES: C[C@H](F)c1cncc(N(Cc2ncc(-c3nnc(C(F)F)o3)s2)S(C)(=O)=O)c1] | 1H NMR (400 MHz, CDCl3) δ 8.68 (s, 1 H), 8.58 (s, 1 H), 8.42 (s, 1 H), 7.84 (s, 1 H), 6.92 (t, J = 51.6 Hz, 1 H), 5.80-5.61 (m, 1 H), 5.29 (s, 2 H), 3.11 (s, 3 H), 1.75-1.55 (m, 3 H). LCMS RT = 0.550 min, m/z = 434.1 | 0.0119 |
| I-217 | 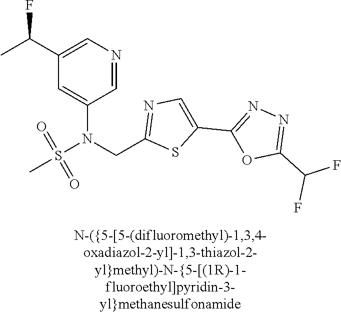 [SMILES: C[C@@H](F)c1cncc(N(Cc2ncc(-c3nnc(C(F)F)o3)s2)S(C)(=O)=O)c1] | 1H NMR (400 MHz, CDCl3) δ 8.70 (s, 1 H), 8.58 (s, 1 H), 8.42 (s, 1 H), 7.86 (s, 1 H), 6.92 (t, J = 51.6 Hz, 1 H), 5.81-5.60 (m, 1 H), 5.29 (s, 2 H), 3.11 (s, 3 H), 1.75-1.55 (m, 3 H). LCMS RT = 0.551 min, m/z = 434.1 | 0.0236 |
| I-218 | 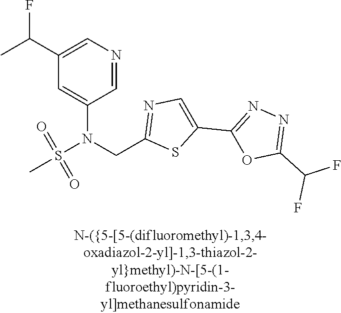 [SMILES: CC(F)c1cncc(N(Cc2ncc(-c3nnc(C(F)F)o3)s2)S(C)(=O)=O)c1] | 1H NMR (400 MHz, CDCl3) δ 8.68 (d, J = 2.4 Hz, 1 H), 8.57 (s, 1 H), 8.41 (s, 1 H), 7.84 (s, 1 H), 6.91 (t, J = 51.2 Hz, 1 H), 5.79- 5.62 (m, 1 H), 5.28 (s, 2 H), 3.11 (s, 3 H), 1.75-1.55 (m, 3 H). LCMS RT = 0.545 min, m/z = 434.1 | 0.0369 |
| I-219 | 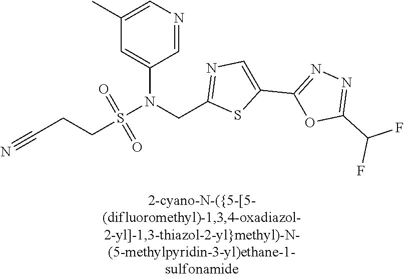 [SMILES: Cc1cncc(N(Cc2ncc(-c3nnc(C(F)F)o3)s2)S(=O)(=O)CCC#N)c1] | 1H NMR (400 MHz, CDCl3) δ 8.52 (d, J = 2.0 Hz, 1 H), 8.47 (s, 1 H), 8.45 (s, 1 H), 7.65 (s, 1 H), 6.92 (t, J = 51.6 Hz, 1 H), 5.28 (s, 2 H), 3.53 (t, J = 7.6 Hz, 2 H), 2.98 (t, J = 7.2 Hz, 2 H), 2.39 (s, 3 H). LCMS RT = 0.507 min, m/z = 441.1 | 0.0202 |
| I-220 | 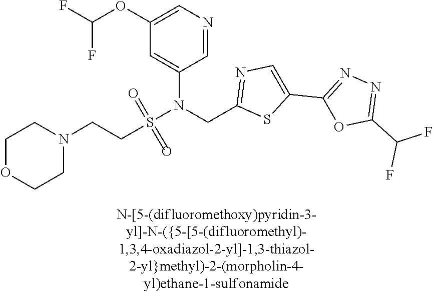 | 1H NMR (400 MHz, CDCl3) δ 8.61 (s, 1 H), 8.46 (s, 1 H), 8.39 (s, 1 H), 7.70 (s, 1 H), 6.91 (t, J = 51.6 Hz, 1 H), 6.59 (t, J = 72.0 Hz, 1 H), 5.31 (s, 2 H), 3.77- 3.69 (m, 4 H), 3.38 (t, J = 6.8 Hz, 2 H), 2.89 (t, J = 7.2 Hz, 2 H), 2.54-2.46 (m, 4 H). LCMS RT = 1.504 min, m/z = 553.2 | 0.0184 |
| I-221 | 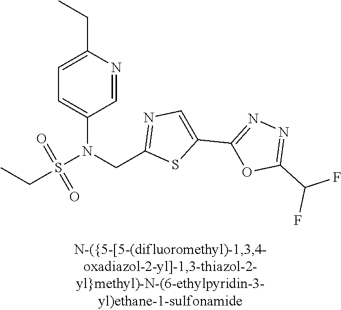 [SMILES: CCc1ccc(N(Cc2ncc(-c3nnc(C(F)F)o3)s2)S(=O)(=O)CC)cn1] | 1H NMR (400 MHz, CDCl3) δ 8.58 (d, J = 2.4 Hz, 1 H), 8.37 (s, 1 H), 7.75-7.70 (m, 1 H), 7.22 (d, J = 8.4 Hz, 1 H), 6.91 (t, J = 51.6 Hz, 1 H), 5.7 (s, 2 H), 3.20 (q, J = 7.6 Hz , 2 H), 2.83 (q, J = 7.6 Hz , 2 H), 1.46 (t, J = 7.2 Hz, 3 H), 1.30 (t, J = 7.6 Hz, 3 H). LCMS RT = 1.134 min, m/z = 430.2 | 0.033 |
| I-222 | 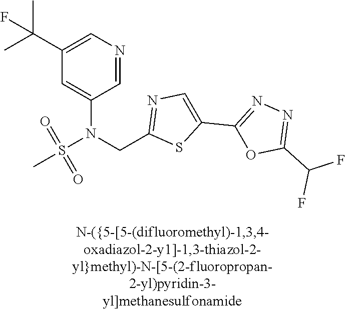 [SMILES: CC(C)(F)c1cncc(N(Cc2ncc(-c3nnc(C(F)F)o3)s2)S(C)(=O)=O)c1] | 1H NMR (400 MHz, CDCl3) δ 8.64 (d, J = 2.0 Hz, 1 H), 8.60 (s, 1 H), 8.42 (s, 1 H), 7.86 (t, J = 2.0 Hz, 1 H), 6.92 (t, J = 51.6 Hz, 1 H), 5.28 (s, 2 H), 3.11 (s, 3 H), 1.75 (s, 3 H), 1.69 (s, 3 H). LCMS RT = 1.499 min, m/z = 447.9 | 0.0376 |
| I-223 | 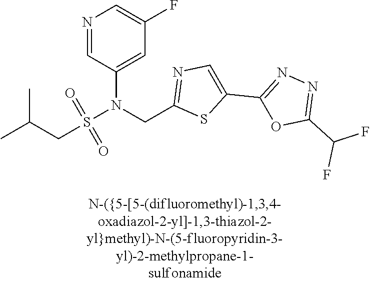 [SMILES: CC(C)CS(=O)(=O)N(Cc1ncc(-c2nnc(C(F)F)o2)s1)c1cncc(F)c1] | 1H NMR (400 MHz, CDCl3) δ 8.58 (s, 1 H), 8.47 (d, J = 2.8 Hz, 1 H), 8.40 (s, 1 H), 7.68-7.62 (m, 1 H), 6.91 (t, J = 51.6 Hz, 1 H), 5.29 (s, 2 H), 3.05 (d, J = 2.0 Hz, 2 H), 2.40-2.30 (m, 1 H), 1.12 (d, J = 6.8 Hz, 6 H). LCMS RT = 1.790 min, m/z = 447.9 | 0.00563 |
| I-224 | 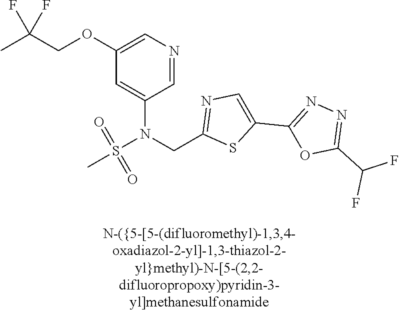 [SMILES: CC(F)(F)COc1cncc(N(Cc2ncc(-c3nnc(C(F)F)o3)s2)S(C)(=O)=O)c1] | 1H NMR (400 MHz, CDCl3) δ 8.42 (s, 1 H), 8.39-8.33 (m, 2 H), 7.48-7.41 (m, 1 H), 6.92 (t, J = 51.6 Hz, 1 H), 5.28 (s, 2 H), 4.18 (t, J = 11.2 Hz, 2 H), 3.11 (s, 3 H), 1.79 (t, J = 18.8 Hz, 3 H). LCMS RT = 1.492 min, m/z = 482.2 | 0.0204 |
| I-225 | 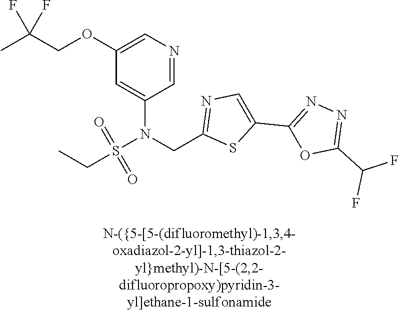 [SMILES: CCS(=O)(=O)N(Cc1ncc(-c2nnc(C(F)F)o2)s1)c1cncc(OCC(C)(F)F)c1] | 1H NMR (400 MHz, CDCl3) δ 8.42-8.36 (m, 2 H), 8.33 (s, 1 H), 7.45 (s, 1 H), 6.91 (t, J = 51.6 Hz, 1 H), 5.30 (s, 2 H), 4.17 (t, J = 11.2 Hz, 2 H), 3.21 (q, J = 7.6 Hz, 2 H), 1.79 (t, J = 18.8 Hz, 3 H), 1.46 (t, J = 7.6 Hz, 3 H). LCMS RT = 1.559 min, m/z = 496.2 | 0.00601 |
| I-226 | 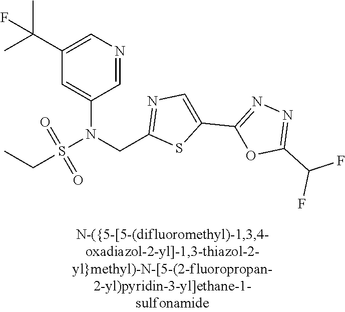 [SMILES: CCS(=O)(=O)N(Cc1ncc(-c2nnc(C(F)F)o2)s1)c1cncc(C(C)(C)F)c1] | 1H NMR (400 MHz, CDCl3) δ 8.66 (d, J = 2.4 Hz, 1 H), 8.59 (d, J = 1.2 Hz, 1 H), 8.40 (s, 1 H), 7.91-7.86 (m, 1 H), 6.91 (t, J = 51.6 Hz, 1 H), 5.30 (s, 2 H), 3.21 (q, J = 6.8 Hz, 2 H), 1.75 (s, 3 H), 1.69 (s, 3 H), 1.47 (t, J = 6.4 Hz, 3 H). LCMS RT = 1.510 min, m/z = 462.2 | 0.0319 |
| I-227 | 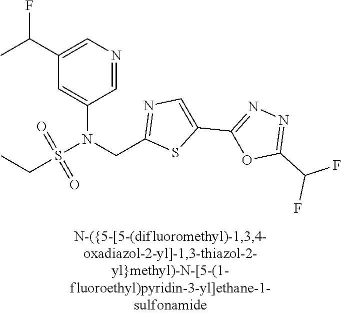 [SMILES: CCS(=O)(=O)N(Cc1ncc(-c2nnc(C(F)F)o2)s1)c1cncc(C(C)F)c1] | 1H NMR (400 MHz, CDCl3) δ 8.67 (d, J = 2.4 Hz, 1 H), 8.56 (s, 1 H), 8.39 (s, 1 H), 7.83 (s, 1 H), 6.91 (t, J = 51.6 Hz, 1 H), 5.79- 5.62 (m, 1 H), 5.30 (s, 2 H), 3.21 (q, J = 7.2 Hz, 2 H), 1.74-1.56 (m, 3 H), 1.47 (t, J = 7.2 Hz, 3 H). LCMS RT = 1.518 min, m/z = 447.9 | 0.0123 |
| I-228 | 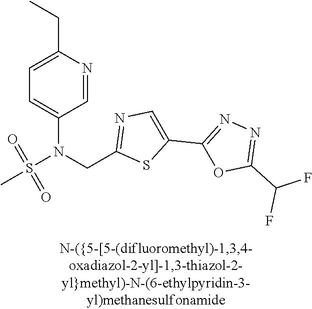 [SMILES: CCc1ccc(N(Cc2ncc(-c3nnc(C(F)F)o3)s2)S(C)(=O)=O)cn1] | 1H NMR (400 MHz, CD3OD) δ 8.54 (d, J = 2.4 Hz, 1 H), 8.39 (s, 1 H), 7.93-7.88 (m, 1 H), 7.35 (d, J = 8.4 Hz, 1 H), 7.17 (t, J = 51.6 Hz, 1 H), 5.31 (s, 2 H), 3.12 (s, 3 H), 2.78 (q, J = 7.6 Hz, 2 H), 1.24 (t, J = 7.6 Hz, 3 H). LCMS RT = 0.981 min, m/z = 416.2 | 0.0435 |
| I-229 | 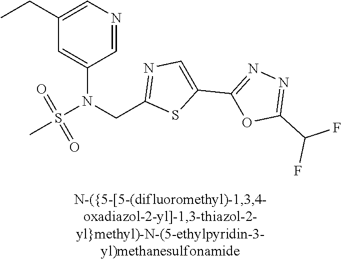 [SMILES: CCc1cncc(N(Cc2ncc(-c3nnc(C(F)F)o3)s2)S(C)(=O)=O)c1] | 1H NMR (400 MHz, CD3OD) δ 8.51 (s, 1 H), 8.42 (s, 1 H), 8.40 (s, 1 H), 7.89 (s, 1 H), 7.18 (t, J = 51.6 Hz, 1 H), 5.35 (s, 2 H), 3.14 (s, 3 H), 2.69 (q, J = 7.2 Hz, 2 H), 1.25 (t, J = 7.6 Hz, 3 H). LCMS RT = 1.099 min, m/z = 416.2 | 0.0248 |
| I-230 | 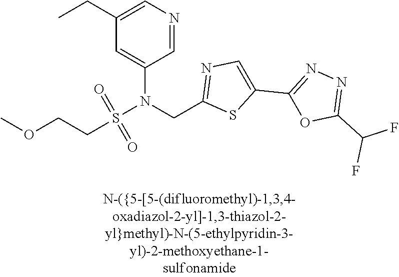 [SMILES: CCc1cncc(N(Cc2ncc(-c3nnc(C(F)F)o3)s2)S(=O)(=O)CCOC)c1] | 1H NMR (400 MHz, CD3OD) δ 8.50 (d, J = 2.4 Hz, 1 H), 8.38 (s, 1 H), 8.35 (s, 1 H), 7.88 (s, 1 H), 7.18 (t, J = 51.6 Hz, 1 H), 5.31 (s, 2 H), 3.82 (t, J = 5.6 Hz, 2 H), 3.48 (t, J = 5.2 Hz, 2 H), 3.43 (s, 3 H), 2.69 (q, J = 5.2 Hz, 2 H), 1.24 (t, J = 7.6 Hz, 3 H). LCMS RT = 1.752 min, m/z = 460.2 | 0.033 |
| I-231 | 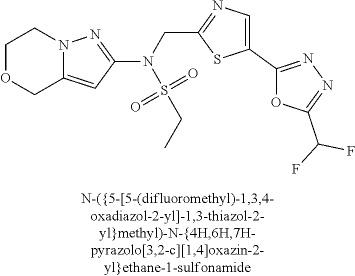 [SMILES: CCS(=O)(=O)N(Cc1ncc(-c2nnc(C(F)F)o2)s1)c1cc2n(n1)CCOC2] | 1H NMR (400 MHz, CDCl3) δ 8.39 (s, 1 H), 6.90 (t, J = 51.6 Hz, 1 H), 6.12 (s, 1 H), 5.38 (s, 2 H), 4.77 (s, 2 H), 4.12-4.06 (m, 4 H), 3.27 (q, J = 7.2 Hz, 2 H), 1.42 (t, J = 7.6 Hz, 3 H). LCMS RT = 0.552 min, m/z = 447.1 | 0.0924 |
| I-232 | 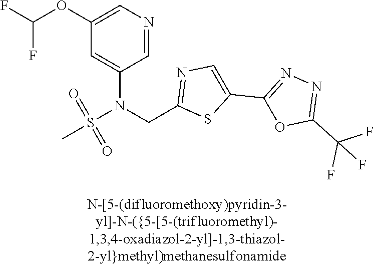 [SMILES: CS(=O)(=O)N(Cc1ncc(-c2nnc(C(F)(F)F)o2)s1)c1cncc(OC(F)F)c1] | 1H NMR (400 MHz, CDCl3) δ 8.63 (d, J = 2.0 Hz, 1 H), 8.51 (d, J = 2.4 Hz, 1 H), 8.46 (s, 1 H), 7.70 (s, 1 H), 6.61 (t, J = 71.6 Hz, 1 H), 5.30 (s, 2 H), 3.13 (s, 3 H). LCMS RT = 1.766 min, m/z = 471.8 | 0.0342 |
| I-233 | 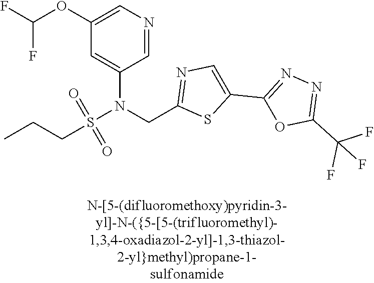 [SMILES: CCCS(=O)(=O)N(Cc1ncc(-c2nnc(C(F)(F)F)o2)s1)c1cncc(OC(F)F)c1] | 1H NMR (400 MHz, CDCl3) δ 8.59 (d, J = 2.0 Hz, 1 H), 8.47 (d, J = 2.4 Hz, 1 H), 8.42 (s, 1 H), 7.67 (t, J = 2.4 Hz, 1 H), 6.59 (t, J = 72.0 Hz, 1 H), 5.29 (s, 2 H), 3.19-3.11 (m, 2 H), 1.97-1.88 (m, 2 H), 1.08 (t, J = 7.6 Hz, 3 H). LCMS RT = 2.013 min, m/z = 499.9 | 0.0606 |
| I-234 | 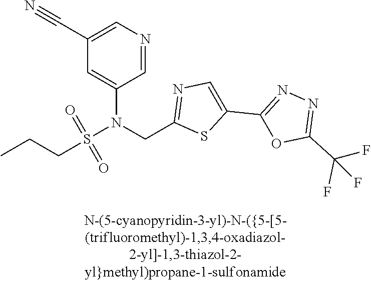 [SMILES: CCCS(=O)(=O)N(Cc1ncc(-c2nnc(C(F)(F)F)o2)s1)c1cncc(C#N)c1] | 1H NMR (400 MHz, CDCl3) δ 8.95 (d, J = 2.4 Hz, 1 H), 8.82 (s, 1 H), 8.44 (s, 1 H), 8.16 (d, J = 2.4 Hz, 1 H), 5.29 (s, 2 H), 3.18 - 3.11 (m, 2 H), 2.00-1.80 (m, 2 H), 1.09 (t, J = 7.2 Hz, 3 H). LCMS RT = 1.811 min, m/z = 459.2 | 0.184 |
| I-235 | 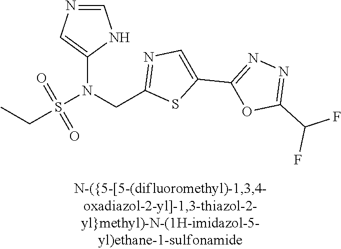 [SMILES: CCS(=O)(=O)N(Cc1ncc(-c2nnc(C(F)F)o2)s1)c1cnc[nH]1] | 1H NMR (400 MHz, CD3OD) δ 8.43 (s, 1 H), 7.99 (s, 1 H), 7.39- 7.03 (m, 2 H), 5.30 (s, 2 H), 3.39- 3.28 (m, 2 H), 1.40 (t, J = 7.2 Hz, 3 H). LCMS RT = 1.848 min, m/z = 390.8 | 0.323 |
| I-236 | 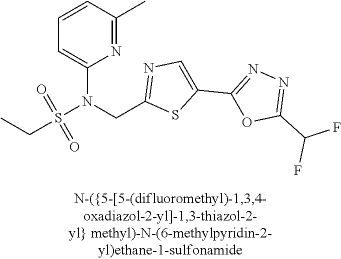 [SMILES: CCS(=O)(=O)N(Cc1ncc(-c2nnc(C(F)F)o2)s1)c1cccc(C)n1] | 1H NMR (400 MHz, CDCl3) δ 8.39 (s, 1 H), 7.61 (t, J = 8.0 Hz, 1 H), 7.45 (d, J = 8.4 Hz, 1 H), 7.01 (s, 1 H), 6.89 (t, J = 51.6 Hz, 1 H), 5.60 (s, 2 H), 3.27 (q, J = 7.6 Hz, 2 H), 2.52 (s, 3 H), 1.37 (t, J = 7.4 Hz, 3 H). LCMS RT = 1.704 min, m/z = 416.2 | 0.0592 |
| I-237 | 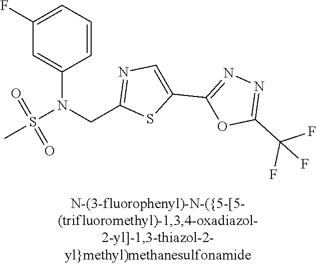 [SMILES: CS(=O)(=O)N(Cc1ncc(-c2nnc(C(F)(F)F)o2)s1)c1cccc(F)c1] | 1H NMR (400 MHz, CDCl3) δ 8.41 (s, 1 H), 7.44-7.36 (m, 1 H), 7.30-7.27 (m, 1 H), 7.24- 7.20 (m, 1 H), 7.12-7.05 (m, 1 H), 5.27 (s, 2 H), 3.07 (s, 3 H). LCMS RT = 1.912 min, m/z = 422.8 | 0.0992 |
| I-238 | 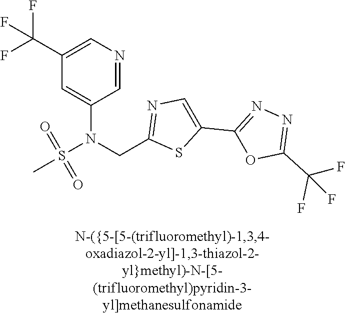 [SMILES: CS(=O)(=O)N(Cc1ncc(-c2nnc(C(F)(F)F)o2)s1)c1cncc(C(F)(F)F)c1] | 1H NMR (400 MHz, CDCl3) δ 8.95 (d, J = 2.0 Hz, 1 H), 8.87 (s, 1 H), 8.46 (s, 1 H), 8.12 (t, J = 2.0 Hz, 1 H), 5.31 (s, 2 H), 3.12 (s, 3 H). LCMS RT = 1.905 min, m/z = 473.8 | 0.256 |
| I-239 | 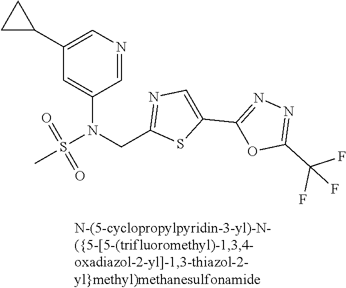 [SMILES: CS(=O)(=O)N(Cc1ncc(-c2nnc(C(F)(F)F)o2)s1)c1cncc(C2CC2)c1] | 1H NMR (400 MHz, CDCl3) δ 8.46 (s, 1 H), 8.42 (s, 1 H), 8.37 (s, 1 H), 7.45 (t, J = 2.4 Hz, 1 H), 5.26 (s, 2 H), 3.09 (s, 3 H), 2.00- 1.90 (m, 1 H), 1.11-1.08 (m, 2 H), 0.78-0.75 (m, 2 H). LCMS RT = 1.621 min, m/z = 445.9 | 0.085 |
| I-240 | 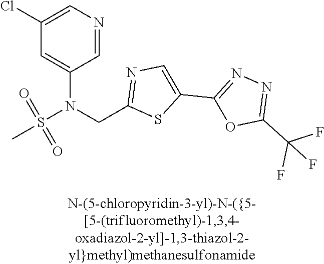 [SMILES: CS(=O)(=O)N(Cc1ncc(-c2nnc(C(F)(F)F)o2)s1)c1cncc(Cl)c1] | 1H NMR (400 MHz, CDCl3) δ 8.62 (s, 1 H), 8.57 (s, 1 H), 7.89 (s, 1 H), 5.28 (s, 2 H), 3.11 (s, 3 H). LCMS RT = 2.259 min, m/z = 440.1 | 0.062 |
| I-241 | 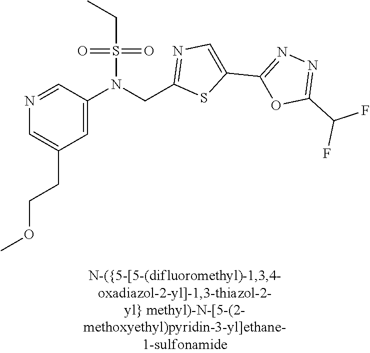 [SMILES: CCS(=O)(=O)N(Cc1ncc(-c2nnc(C(F)F)o2)s1)c1cncc(CCOC)c1] | 1H NMR (400 MHz, CDCl3) δ 8.50 (s, 1 H), 8.48 (s, 1 H), 7.80 (s, 1 H), 7.65 (s, 1 H), 6.92 (t, J = 51.6 Hz, 1 H), 5.80 (s, 2 H), 3.60 (t, J = 6.0 Hz, 2 H), 3.33 (s, 3 H), 3.08 (q, J = 7.2 Hz, 2 H), 2.85 (t, J = 6.0 Hz, 2 H), 1.40 (t, J = 7.6 Hz, 3 H). LCMS RT = 0.718 min, m/z = 460.2 | 0.994 |
| I-242 | 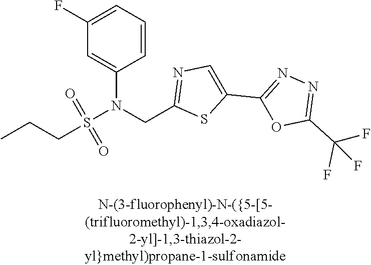 [SMILES: CCCS(=O)(=O)N(Cc1ncc(-c2nnc(C(F)(F)F)o2)s1)c1cccc(F)c1] | 1H NMR (400 MHz, CDCl3) δ 8.41 (s, 1 H), 7.43-7.37 (m, 1 H), 7.27-7.26 (m, 1 H), 7.25- 7.22 (m, 1 H), 7.10-7.05 (m, 1 H), 5.30 (s, 2 H), 3.15-3.10 (m, 2 H), 1.97-1.89 (m, 2 H), 1.09 (t, J = 7.2 Hz, 3 H). LCMS RT = 2.207 min, m/z = 450.8 | 0.053 |
| I-243 | 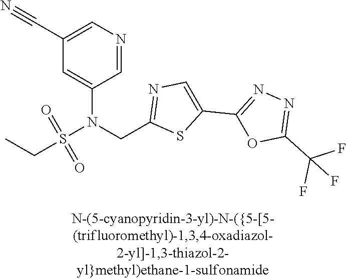 [SMILES: CCS(=O)(=O)N(Cc1ncc(-c2nnc(C(F)(F)F)o2)s1)c1cncc(C#N)c1] | 1H NMR (400 MHz, CDCl3) δ 8.96 (d, J = 2.4 Hz, 1 H), 8.82 (d, J = 2.0 Hz, 1 H), 8.44 (s, 1 H), 8.17 (t, J = 2.0 Hz, 1 H), 5.30 (s, 2 H), 3.21 (q, J = 7.2 Hz, 2 H), 1.45 (t, J = 7.2 Hz, 3 H). LCMS RT = 1.718 min, m/z = 444.8 | 0.129 |
| I-244 | 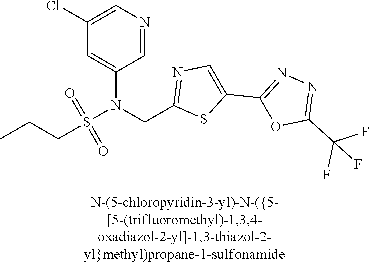 [SMILES: CCCS(=O)(=O)N(Cc1ncc(-c2nnc(C(F)(F)F)o2)s1)c1cncc(Cl)c1] | 1H NMR (400 MHz, CDCl3) δ 8.61 (d, J = 2.4 Hz, 1 H), 8.55 (d, J = 2.4 Hz, 1 H), 8.42 (s, 1 H), 7.88 (t, J = 2.0 Hz, 1 H), 5.28 (s, 2 H), 3.19-3.11 (m, 2 H), 1.97- 1.82 (m, 2 H), 1.09 (t, J = 7.6 Hz, 3 H). LCMS RT = 2.027 min, m/z = 467.8 | 0.0872 |
| I-245 | 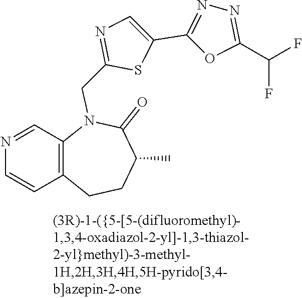 [SMILES: C[C@@H]1CCc2ccncc2N(Cc2ncc(-c3nnc(C(F)F)o3)s2)C1=O] | 1H NMR (400 MHz, CDCl3) δ 8.96 (s, 1 H), 8.52 (d, J = 5.2 Hz, 1 H), 8.38 (s, 1 H), 7.52 (d, J = 5.2 Hz, 1 H), 6.91 (t, J = 51.6 Hz, 1 H), 5.47-5.27 (m, 2 H), 3.09- 3.04 (m, 1 H), 2.83-2.78 (m, 1 H), 2.51-2.47 (m, 1 H), 2.26- 2.21 (m, 1 H), 2.18-2.11 (m, 1 H), 1.16 (d, J = 6.4 Hz, 3 H) LCMS RT = 1.250 min, m/z = 392.3. | 1.42 |
| I-246 | 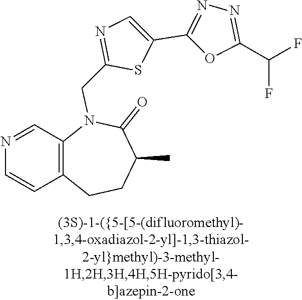 [SMILES: C[C@H]1CCc2ccncc2N(Cc2ncc(-c3nnc(C(F)F)o3)s2)C1=O] | 1H NMR (400 MHz, CDCl3) δ 8.91 (s, 1 H), 8.50 (d, J = 5.2 Hz, 1 H), 8.37 (s, 1 H), 7.47 (d, J = 5.2 Hz, 1 H), 6.91 (t, J = 51.6 Hz, 1 H), 5.46-5.28 (m, 2 H), 3.11- 2.85 (m, 1 H), 2.84-2.71 (m, 1 H), 2.548-2.45 (m, 1 H), 2.29- 2.14 (m, 1 H), 2.18-2.07 (m, 1 H), 1.16 (d, J = 6.4 Hz, 3 H) LCMS RT = 1.251 min, m/z = 392.3. | 0.039 |
| I-247 | 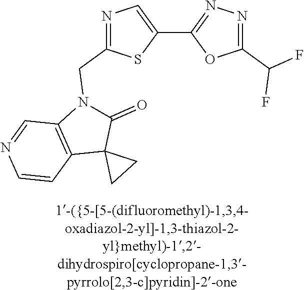 [SMILES: O=C1N(Cc2ncc(-c3nnc(C(F)F)o3)s2)c2cnccc2C12CC2] | 1H NMR (400 MHz, CDCl3) δ 8.47 (s, 1 H), 8.44-8.40 (m, 2 H), 7.07 (d, J = 5.2 Hz, 1 H), 6.90 (t, J = 51.6 Hz, 1 H), 5.43 (s, 2 H), 2.17-2.10 (m, 2 H), 1.93- 1.86 (m, 2 H) LCMS RT = 0.970 min, m/z = 376.2. | 0.158 |
| I-248 | 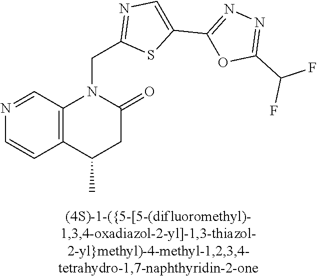 [SMILES: C[C@H]1CC(=O)N(Cc2ncc(-c3nnc(C(F)F)o3)s2)c2cnccc21] | 1H NMR (400 MHz, CDCl3) δ 8.54 (s, 1 H), 8.41 (s, 1 H), 8.35 (d, J = 4.8 Hz, 1 H), 7.19 (d, J = 4.8 Hz, 1 H), 6.89 (t, J = 51.6 Hz, 1 H), 5.62-5.46 (m, 2 H), 3.23- 3.16 (m, 1 H), 2.92-2.84 (m, 1 H), 2.66-2.55 (m, 1 H), 1.38 (d, J = 6.8 Hz, 3 H) LCMS RT =1.098 min, m/z = 378.3. | 0.034 |
| I-249 | 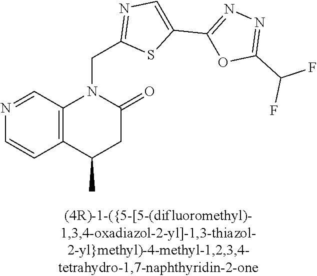 [SMILES: C[C@@H]1CC(=O)N(Cc2ncc(-c3nnc(C(F)F)o3)s2)c2cnccc21] | 1H NMR (400 MHz, CDCl3) δ 8.53 (s, 1 H), 8.40 (s, 1 H), 8.34 (d, J = 4.8 Hz, 1 H), 7.18 (d, J = 4.8 Hz, 1 H), 6.90 (t, J = 51.6 Hz, 1 H), 5.60-5.46 (m, 2 H), 3.23- 3.15 (m, 1 H), 2.90-2.81 (m, 1 H), 2.64-2.55 (m, 1 H), 1.38 (d, J = 6.8 Hz, 3 H) LCMS RT = 1.028 min, m/z = 378.2. | 0.089 |
| I-250 | 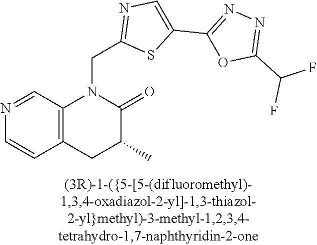 [SMILES: C[C@@H]1Cc2ccncc2N(Cc2ncc(-c3nnc(C(F)F)o3)s2)C1=O] | 1H NMR (400 MHz, CDCl3) δ 8.54 (s, 1 H), 8.41 (s, 1 H), 8.31 (d, J = 3.6 Hz, 1 H), 7.18 (d, J = 4.4 Hz, 1 H), 6.89 (t, J = 51.6 Hz, 1 H), 5.62-5.36 (m, 2 H), 3.09- 3.02 (m, 1 H), 2.89-2.79 (m, 2 H), 1.35 (d, J = 6.8 Hz, 3 H). LCMS RT = 0.684 min, m/z = 378.2. | 0.041 |
| I-251 | 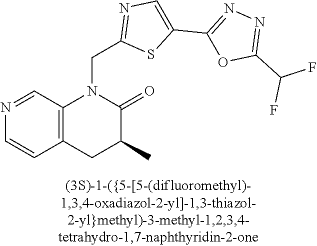 [SMILES: C[C@H]1Cc2ccncc2N(Cc2ncc(-c3nnc(C(F)F)o3)s2)C1=O] | 1H NMR (400 MHz, CDCl3) δ 8.52 (s, 1 H), 8.41 (s, 1 H), 8.30 (d, J = 4.4 Hz, 1 H), 7.17 (d, J = 4.8 Hz, 1 H), 6.89 (t, J = 51.6 Hz, 1 H), 5.62-5.36 (m, 2 H), 3.09- 3.02 (m, 1 H), 2.89-2.79 (m, 2 H), 1.34 (d, J = 6.8 Hz, 3 H). LCMS RT = 0.521 min, m/z = 378.2. | 0.034 |
| I-252 | 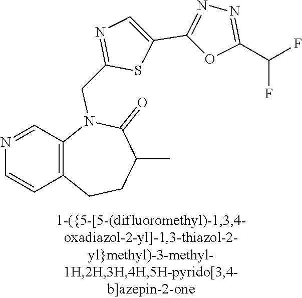 [SMILES: CC1CCc2ccncc2N(Cc2ncc(-c3nnc(C(F)F)o3)s2)C1=O] | 1H NMR (400 MHz, CDCl3) δ 8.77 (s, 1 H), 8.46 (d, J = 4.8 Hz, 1 H), 8.37 (s, 1 H), 7.30 (d, J = 4.4 Hz, 1 H), 6.90 (t, J = 51.6 Hz, 1 H), 5.37 (s, 2 H), 2.99-2.90 (m, 1 H), 2.70-2.66 (m, 1 H), 2.55-2.49 (m, 1 H), 2.23-2.14 (m, 1 H), 2.18-2.04 (m, 1 H), 1.16 (d, J = 6.4 Hz, 3 H). LCMS RT = 1.229 min, m/z = 392.3. | 0.073 |
| I-253 | 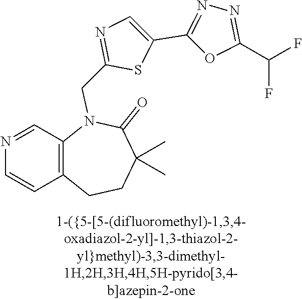 [SMILES: CC1(C)CCc2ccncc2N(Cc2ncc(-c3nnc(C(F)F)o3)s2)C1=O] | 1H NMR (400 MHz, CDCl3) δ 8.84 (s, 1 H), 8.47 (s, 1 H), 8.40 (s, 1 H), 7.42 (s, 1 H), 6.91 (t, J = 51.6 Hz, 1 H), 5.37 (s, 2 H), 2.91 (t, J = 6.0 Hz, 2 H), 2.18 (t, J = 6.4 Hz, 2 H), 1.04 (s, 6 H) LCMS RT = 1.946 min, m/z = 405.9. | 0.035 |
| I-254 | 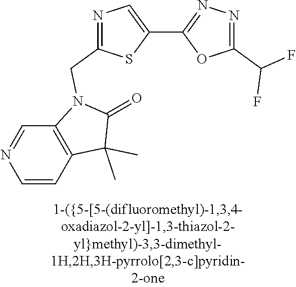 [SMILES: CC1(C)C(=O)N(Cc2ncc(-c3nnc(C(F)F)o3)s2)c2cnccc21] | 1H NMR (400 MHz, CDCl3) δ 8.45-8.43 (m, 2 H), 8.29 (s, 1 H), 7.24 (d, J = 4.8 Hz, 1 H), 6.89 (t, J = 51.6 Hz, 1 H), 5.33 (s, 2 H), 1.49 (s, 6 H) LCMS RT = 0.408 min, m/z = 378.1. | 0.149 |
| 1-255 | 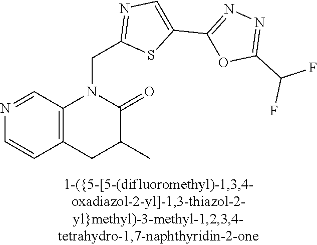 [SMILES: CC1Cc2ccncc2N(Cc2ncc(-c3nnc(C(F)F)o3)s2)C1=O] | 1H NMR (400 MHz, CDCl3) δ 8.59 (s, 1 H), 8.42 (s, 1 H), 8.34 (d, J = 4.8 Hz, 1 H), 7.26 (d, J = 4.8 Hz, 1 H), 6.90 (t, J = 51.6 Hz, 1 H), 5.63-5.37 (m, 2 H), 3.13- 3.06 (m, 1 H), 2.92-2.80 (m, 2 H), 1.36 (d, J = 6.4 Hz, 3 H) LCMS RT = 0.583 min, m/z = 378.2. | 0.036 |
| I-256 | 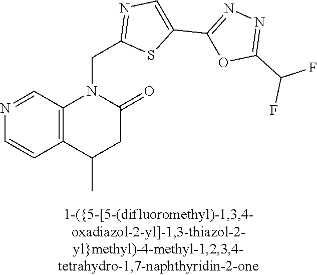 [SMILES: CC1CC(=O)N(Cc2ncc(-c3nnc(C(F)F)o3)s2)c2cnccc21] | 1H NMR (400 MHz, CDCl3) δ 8.58 (s, 1 H), 8.45-8.34 (m, 2 H), 7.25 (d, J = 4.8 Hz, 1 H), 6.89 (t, J = 51.6 Hz, 1 H), 5.62-5.47 (m, 2 H), 3.27-3.20 (m, 1 H), 2.94-2.82 (m, 1 H), 2.67-2.56 (m, 1 H), 1.40 (d, J = 6.8 Hz, 3 H) LCMS RT = 0.580 min, m/z = 378.2. | 0.057 |
| I-257 | 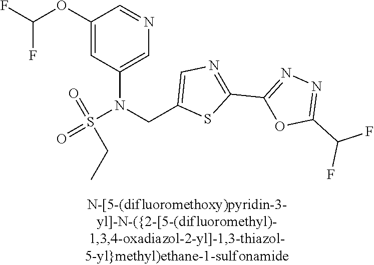 [SMILES: CCS(=O)(=O)N(Cc1cnc(-c2nnc(C(F)F)o2)s1)c1cncc(OC(F)F)c1] | 1H NMR (400 MHz, CD3OD) δ 8.48 (d, J = 2.4 Hz, 1 H), 8.40 (d, J = 2.4 Hz, 1 H), 7.90 (s, 1 H), 7.77 (t, J = 2.4 Hz, 1 H), 7.23 (t, J = 51.6 Hz, 1 H), 6.97 (t, J = 72.8 Hz, 1 H), 5.35 (s, 2 H), 3.31 (q, J = 7.6 Hz, 2 H), 1.41 (t, J = 7.6 Hz, 3 H) LCMS RT = 1.251 min, m/z = 468.2. | 0.231 |
| I-258 | 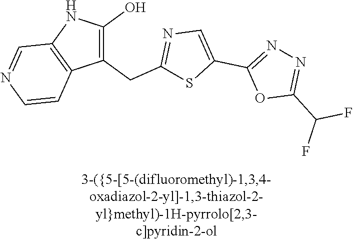 [SMILES: Oc1[nH]c2cnccc2c1Cc1ncc(-c2nnc(C(F)F)o2)s1] | 1H NMR (400 MHz, CD3OD) δ 8.40 (s, 1 H), 7.65 (s, 1 H), 7.50 (d, J = 6.8 Hz, 1 H), 7.17 (t, J = 51.6 Hz, 1 H), 6.94 (d, J = 6.8 Hz, 1 H), 4.27 (s, 2 H) LCMS RT = 0.778 min, m/z = 350.2. | 0.069 |
| I-259 | 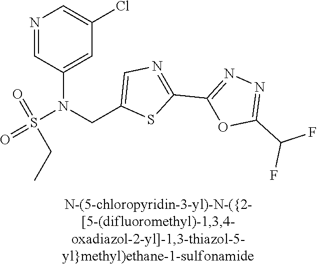 [SMILES: CCS(=O)(=O)N(Cc1cnc(-c2nnc(C(F)F)o2)s1)c1cncc(Cl)c1] | 1H NMR (400 MHz, CD3OD) δ 8.55-8.51 (m, 2 H), 8.05 (t, J = 2.4 Hz, 1 H), 7.92 (s, 1 H), 7.25 (t, J = 51.6 Hz, 1 H), 5.36 (s, 2 H), 3.34 (q, J = 7.2 Hz, 2 H), 1.42 (t, J = 7.6 Hz, 3 H) LCMS RT = 1.273 min, m/z = 436.2. | 0.121 |
| I-260 | 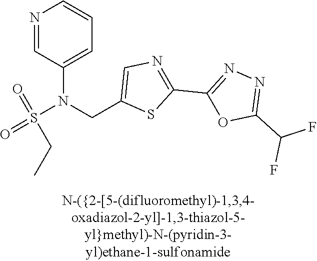 [SMILES: CCS(=O)(=O)N(Cc1cnc(-c2nnc(C(F)F)o2)s1)c1cccnc1] | 1H NMR (400 MHz, CDCl3) δ 8.58 (d, J = 2.4 Hz, 1 H), 8.52- 8.48 (m, 1 H), 7.98 (s, 1 H), 7.87- 7.66 (m, 1 H), 7.56 (t, J = 51.2 Hz, 1 H), 7.45-7.42 (m, 1 H), 5.34 (s, 2 H), 3.37 (q, J = 7.2 Hz, 2 H), 1.30 (t, J = 7.2 Hz, 3 H) LCMS RT = 0.821 min, m/z = 402.2. | 0.333 |
| I-261 | 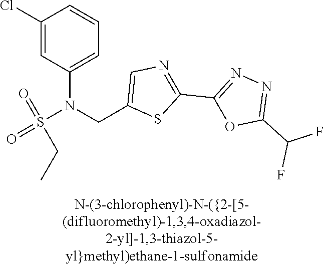 [SMILES: CCS(=O)(=O)N(Cc1cnc(-c2nnc(C(F)F)o2)s1)c1cccc(Cl)c1] | 1H NMR (400 MHz, CDCl3) δ 8.00 (s, 1 H), 7.56 (t, J = 51.2 Hz, 1 H), 7.41-7.36 (m, 4 H), 5.32 (s, 2 H), 3.36 (q, J = 7.2 Hz, 2 H), 1.29 (t, J = 8.0 Hz, 3 H) LCMS RT = 1.554 min, m/z = 435.1. | 0.089 |
| I-262 | 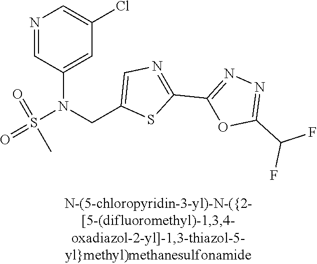 [SMILES: CS(=O)(=O)N(Cc1cnc(-c2nnc(C(F)F)o2)s1)c1cncc(Cl)c1] | 1H NMR (400 MHz, CD3OD) δ 8.61-8.47 (m, 2 H), 8.05 (t, J = 2.4 Hz, 1 H), 7.93 (s, 1 H), 7.23 (t, J = 51.6 Hz, 1 H), 5.32 (s, 2 H), 3.16 (s, 3 H) LCMS RT = 1.643 min, m/z = 422.1. | 0.110 |
| I-263 | 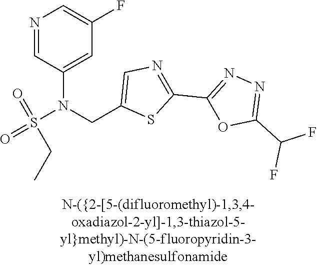 | 1H NMR (400 MHz, CD3OD) δ 8.48 (s, 1 H), 8.46 (d, J = 2.4 Hz, 1 H), 7.93 (s, 1 H), 7.88-7.82 (m, 1 H), 7.23 (t, J = 51.6 Hz, 1 H), 5.33 (s, 2 H), 3.16 (s, 3 H) LCMS RT = 1.478 min, m/z = 406.1. | 0.108 |
| I-264 | 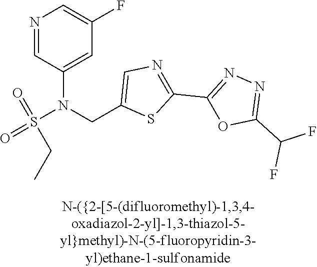 [SMILES: CCS(=O)(=O)N(Cc1cnc(-c2nnc(C(F)F)o2)s1)c1cncc(F)c1] | 1H NMR (400 MHz, CD3OD) δ 8.48 (s, 1 H), 8.45 (d, J = 2.8 Hz, 1 H), 7.91 (s, 1 H), 7.86-7.80 (m, 1 H), 7.24 (t, J = 51.6 Hz, 1 H), 5.35 (s, 2 H), 3.31 (q, J = 7.2 Hz, 2 H), 1.41 (t, J = 7.6 Hz, 3 H). LCMS RT = 1.618 min, m/z = 420.2. | 0.087 |
| I-265 | 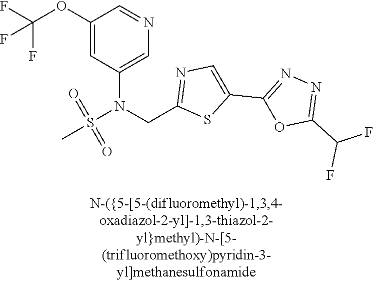 [SMILES: CS(=O)(=O)N(Cc1ncc(-c2nnc(C(F)F)o2)s1)c1cncc(OC(F)(F)F)c1] | 1H NMR (400 MHz, CDCl3) δ 8.72 (d, J = 2.0 Hz, 1 H), 8.56 (s, 1 H), 8.44 (s, 1 H), 7.80 (s, 1 H), 6.92 (t, J = 51.6 Hz, 1 H), 5.30 (s, 2 H), 3.11 (s, 3 H) LCMS RT = 1.950 min, m/z = 472.1. | 0.048 |
| I-266 | 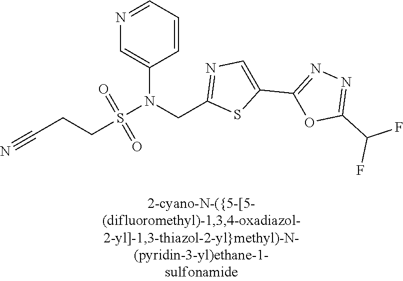 [SMILES: N#CCCS(=O)(=O)N(Cc1ncc(-c2nnc(C(F)F)o2)s1)c1cccnc1] | 1H NMR (400 MHz, CD3OD) δ 8.70 (d, J = 2.8 Hz, 1 H), 8.52 (d, J = 3.6 Hz, 1 H), 8.44 (s, 1 H), 8.07 - 8.01 (m, 1 H), 7.54-7.49 (m, 1 H), 7.21 (t, J = 51.6 Hz, 1 H), 5.41 (s, 2 H), 3.72 (t, J = 7.2 Hz, 2 H), 3.05 (t, J = 7.2 Hz, 2 H). LCMS RT = 1.287 min, m/z = 426.8. | 0.042 |
| I-267 | 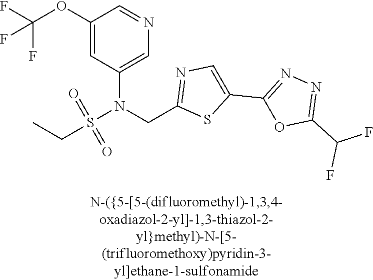 [SMILES: CCS(=O)(=O)N(Cc1ncc(-c2nnc(C(F)F)o2)s1)c1cncc(OC(F)(F)F)c1] | 1H NMR (400 MHz, CDCl3) δ 8.71 (s, 1 H), 8.54 (s, 1 H), 8.41 (s, 1 H), 7.79 (s, 1 H), 6.92 (t, J = 51.6 Hz, 1 H), 5.30 (s, 2 H), 3.22 (q, J = 7.6 Hz, 2 H), 1.45 (t, J = 7.6 Hz, 3 H) LCMS RT = 0.328 min, m/z = 486.1 | 0.044 |
| I-268 | 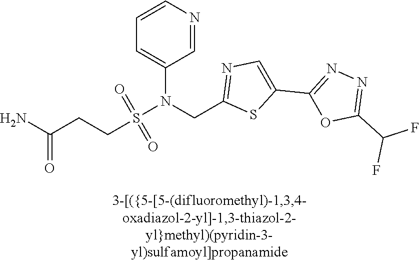 [SMILES: NC(=O)CCS(=O)(=O)N(Cc1ncc(-c2nnc(C(F)F)o2)s1)c1cccnc1] | 1H NMR (400 MHz, CDCl3) δ 8.75 (d, J = 2.4 Hz, 1 H), 8.62- 8.55 (m, 1 H), 8.39 (s, 1 H), 7.90- 7.84 (m, 1 H), 7.40-7.33 (m, 1 H), 6.91 (t, J = 52.0 Hz, 1 H), 5.27 (s, 2 H), 3.58 (t, J = 7.2 Hz, 2 H), 2.81 (t, J = 7.2 Hz, 2 H). LCMS RT = 0.783 min, m/z = 444.9. | 0.013 |
| I-269 | 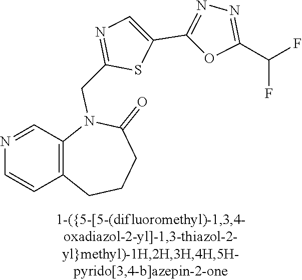 [SMILES: O=C1CCCc2ccncc2N1Cc1ncc(-c2nnc(C(F)F)o2)s1] | 1H NMR (400 MHz, CDCl3) δ 8.77 (s, 1 H), 8.46 (s, 1 H), 8.38 (s, 1 H), 7.28 (s, 1 H), 6.91 (t, J = 51.6 Hz, 1 H), 5.36 (s, 2 H), 2.84 (t, J = 6.8 Hz, 2 H), 2.49-2.37 (m, 2 H), 2.37-2.23 (m, 2 H). LCMS RT = 1.043 min, m/z = 378.2 | 0.018 |
| I-270 | 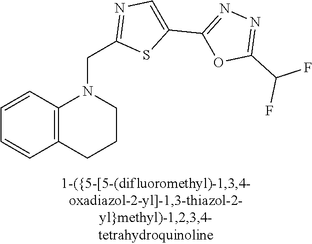 [SMILES: FC(F)c1nnc(-c2cnc(CN3CCCc4ccccc43)s2)o1] | 1H NMR (400 MHz, DMSO-d6) δ 8.57 (s, 1 H), 7.51 (t, J = 51.2 Hz, 1 H), 7.00-6.90 (m, 2 H), 6.60-6.55 (m, 2 H), 4.86 (s, 2 H), 3.47 (t, J = 5.6 Hz, 2 H), 2.75 (t, J = 6.0 Hz, 2 H), 2.02-1.92 (m, 2 H) LCMS RT = 1.769 min, m/z = 349.2 | 0.539 |

### Example 3

### Biochemical Activity and Potency of Various HDAC6 Inhibitors of Formula (II)

The compounds disclosed herein, in particular those of Formula (II), were synthesized according to methods disclosed in WO2021067859, which is incorporated herein by reference in its entirety. These compounds were tested for potency against HDAC6 and selectivity against HDAC1 in a biochemical assay. A biochemical assay was adopted using a luminescent HDAC-Glo I/II assay (Promega) and measured the relative activity of HDAC6 and HDAC1 recombinant proteins. Compounds were first incubated in the presence of HDAC6 or HDAC1 separately, followed by addition of the luminescent substrate. The data was acquired using a plate reader and the biochemical IC₅₀ were calculated from the data accordingly. Data is tabulated in Table 4. From these studies, it was determined that the compounds of the present disclosure are selective inhibitors of HDAC6 over HDAC1, providing selectivity ratios from about 5 to about 30,0000.

TABLE 4
Evaluation of HDAC6 Activity and
Selectivity for Disclosed Compounds.

| Compound ID | HDAC6 IC50 (nM) |
|---|---|
| 1 | 136 |
| I-1 | 12.5 |
| I-6 | 1.61 |
| I-4 | 16.7 |
| I-24 | 146 |
| I-5 | 6.68 |
| I-7A | 1.5 |
| I-7B | 4.13 |
| I-2 | 104 |
| I-10 | 76.6 |
| I-3 | 73 |
| IV-1 | 8.26 |
| IV-4 | 1.8 |
| I-9A | 0.351 |
| IV-2 | 0.677 |
| IV-3 | 3.35 |
| I-9B | 0.791 |
| I-11 | 0.639 |
| I-19 | 0.425 |
| I-18 | 1.68 |
| I-16 | 1.61 |
| I-8B | 0.275 |
| I-13 | 73 |
| I-14 | 28.3 |
| I-17 | 1.12 |
| 4 | 1.2 |
| I-25 | 0.669 |
| IV-9 | 0.595 |
| I-21 | 0.601 |
| I-22 | 3.36 |
| I-23 | 1.59 |
| III-1 | 1.79 |
| IV-5 | 2.04 |
| I-12B | 0.809 |
| IV-10 | 2.3 |
| IV-7 | 1.1 |
| IV-6 | 4.06 |
| IV-8 | 10.3 |
| I-15 | 2.64 |
| I-20 | 3.78 |
| I-8A | 1.65 |
| 5 | 1.49 |
| I-26A | 4.57 |
| I-26B | 2.23 |
| 6 | 2.54 |
| I-27 | 1.13 |
| I-28A | 1.81 |
| I-29 | 13.2 |
| I-30A | 1.36 |
| I-30B | 7.91 |
| I-31 | 13.8 |
| I-32 | 13 |
| I-33 | 27.9 |
| I-34 | 2.32 |
| I-35 | 21.7 |
| I-36 | 5.22 |

The structures, chemical names and additional biochemical properties of the compounds described in this example are provided below.

| Aldehyde/ | | |
|---|---|---|
| Organometallic | Compound | Characterization Data |
| cyclobutanone/ 2- bromopyridine, and n-BuLi | 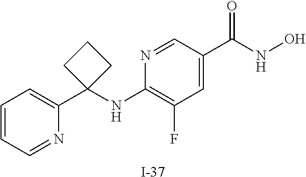 | ¹H NMR (400 MHz, MeOH-d4) δ 8.52 (d, J = 6.03 Hz, 1 H), 8.03 (s, 1 H), 7.70 (t, J = 7.70 Hz, 1 H), 7.58 (d, J = 12.2 Hz, 1 H), 7.51 (d, J = 8.07 Hz, 1 H), 7.22 (t, J = 6.03 Hz, 1 H), 2.89- 2.80 (m, 2 H), 2.62-2.52 (m, 2 H), 2.20-2.15 (m, 1 H), 2.12-2.06 (m, 1H). LC-MS t_{R} (min) 1.24 (20-100% ACN with 0.1 % TFA 6 min), m/z [M + H]⁺ C₁₅H₁₅FN₄O₂ requires: 302.3, found: 303.1 HPLC t_{R} (min) 3.68, 95% (20-100% ACN with 0.1 % TFA 10 min. |
| Amine | Compound | Characterization Data |
| 2-amino-N, 2- dimethyl propanamide | 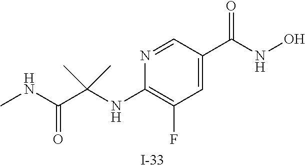 | ¹H NMR (400 MHz, MeOH-d4) δ 8.24 (s, 1 H), 7.62 (s, 1 H), 7.59 (s, 1 H), 2.69-2.65 (m, 3 H), 1.59 (s, 6 H). LC-MS: t_{R} (min) 1.32 (20-100% ACN with 0.1 % TFA 6 min), m/z [M + H]⁺ C₁₁H₁₅FN₄O₂ requires: 270.3, found: 271.1 HPLC t_{R} (min) 1.57, 95% (20-100% ACN with 0.1 % TFA 10 min.) |
| 3-amino-1- methylpiperidin- 2-one | 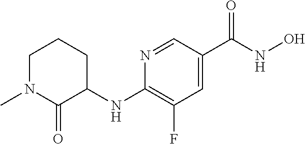 [SMILES: CN1CCCC(Nc2ncc(C(=O)NO)cc2F)C1=O] | ¹H NMR (400 MHz, DMSO-d6) δ 11.1-10.9 (br, s, 1 H), 8.98 (s, 1 H), 8.25 (s, 1 H), 7.73-7.53 (m, 1 H), 7.29-7.04 (m, 1 H), 4.80-4.50 (m, 1 H), 3.33-3.28 (m, 2 H), 2.83 (s, 3 H), 2.16-2.00 (m, 1 H), 1.90 (br, s, 3 H). LC-MS: t_{R} (min) 1.28 (20-100% ACN with 0.1 % TFA 6 min), m/z [M + H]⁺ C₁₂H₁₅FN₄O₂ requires: 282.3, found: 283.1 HPLC t_{R} (min) 3.66, 95% (20-100% ACN with 0.1 % TFA 10 min.) |
| | I-35 | |
| (S)-1- cyclopropylethan- 1-amine | 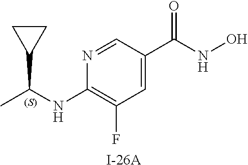 | ¹H NMR (400 MHz, MeOH-d4) δ 7.98 (s, 1 H), 7.32 (d, J = 1.71 Hz, 1 H), 7.29 (d, J = 1.71 Hz, 1 H), 3.45- 3.35 (m, 1 H), 1.07 (d, J = 6.60 Hz, 3 H), 0.86-0.74 (m, 1 H), 0.33-0.01 (m, 4 H). LC-MS: t_{R} (min) 2.03 (20-100% ACN with 0.1 % TFA 6 min), m/z [M + H]⁺ C₁₁H₁₂FN₃O₂ requires: 239.3, found: 240.1 HPLC t_{R} (min) 4.37, 95% (20-100% ACN with 0.1 % TFA 10 min.) |
| (R)-1- cyclopropylethan- 1-amine | 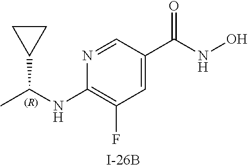 | ¹H NMR (400 MHz, MeOH-d4) δ 7.98 (s, 1 H), 7.32 (d, J = 1.71 Hz, 1 H), 7.29 (d, J = 1.47 Hz, 1 H), 3.10- 3.07 (m, 1 H), 1.07 (d, J = 6.60 Hz, 3 H), 0.90-0.70 (m, 1 H), 0.33-0.00 (m, 4 H). LC-MS: t_{R} (min) 2.03 (20-100% ACN with 0.1 % TFA 6 min), m/z [M + H]⁺ C₁₁H₁₂FN₃O₂ requires: 239.3, found: 240.1 HPLC t_{R} (min) 3.74, 95% (20-100% ACN with 0.1 % TFA 10 min.) |
| (R)-1- phenylpropan-1- amine | 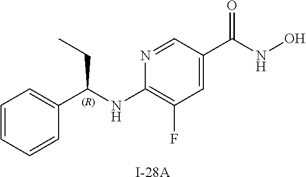 | ¹H NMR (400 MHz, MeOH-d4) δ 8.20 (s, 1 H), 7.56 (d, J = 11.7 Hz, 1 H), 7.39 (d, J = 7.58 Hz, 2 H), 7.29 (t, J = 7.46 Hz, 2 H), 7.24-7.11 (m, 1 H), 5.15-5.02 (m, 1 H), 2.00-1.75 (m, 2 H), 1.02-0.90 (m, 3 H). LC-MS: t_{R} (min) 3.70 (20-100% ACN with 0.1 % TFA 6 min), m/z [M + H]⁺ C₁₃H₁₅FN₃O₂ requires: 289.3, found: 290.1 HPLC t_{R} (min) 5.23, 98% (20-100% ACN with 0.1 % TFA 10 min.) |
| (R)-1- cyclohexylethylamine | 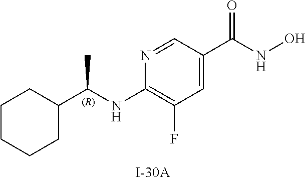 | ¹H NMR (400 MHz, MeOH-d4) δ 8.24 (s, 1H), 7.56 (s, 1 H), 7.53 (s, 1 H), 4.63 (s, 2 H), 4.09 (t, J = 6.72 Hz, 1 H), 1.88-1.65 (m, 5 H), 1.55-1.45 (m, 1 H), 1.31-1.22 (m, 3 H), 1.22-1.15 (m, 3 H), 1.10-0.90 (m, 2 H). LC-MS: t_{R} (min) 3.37 (20-100% ACN with 0.1 % TFA 6 min), m/z [M + H]⁺ C₁₄H₂₀FN₃O₂ requires: 281.3, found: 282.2 HPLC t_{R} (min) 4.84, 97% (20-100% ACN with 0.1 % TFA 10 min.) |
| (S)-1- cyclohexylethylamine | 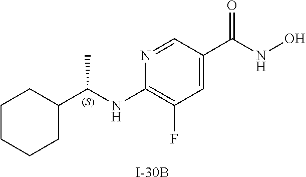 | ¹H NMR (400 MHz, MeOH-d4) δ 8.24 (s, 1H), 7.56 (s, 1 H), 7.53 (s, 1 H), 4.63 (s, 2 H), 4.09 (t, J = 6.97 Hz, 1 H), 1.88-1.65 (m, 5 H), 1.51 (s, 1 H), 1.31-1.20 (m, 3 H), 1.22-1.18 (m, 3 H), 1.10-0.90 (m, 2 H). LC-MS: t_{R} (min) 3.50 (20-100% ACN with 0.1 % TFA 6 min), m/z [M + H]⁺ C₁₄H₂₀FN₃O₂ requires: 281.3, found: 282.1 HPLC t_{R} (min) 5.10, 98% (20-100% ACN with 0.1 % TFA 10 min.) |
| (1S, 2S)-2- methoxycyclopentylamine | 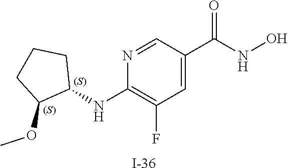 | ¹H NMR (400 MHz, CDCl₃-d) δ 8.27 (s, 1 H), 7.58- 7.39 (m, 1 H), 5.06 (br, s, 1 H), 4.31 (br, s, 1 H), 3.68 (br, s, 1 H), 3.34 (s, 3 H), 2.25- 2.15 (m, 1 H), 1.93- 1.88 (m, 1 H), 1.85-1.75 (m, 3 H), 1.50-1.40 (m, 1 H). LC-MS: t_{R} (min) 1.93 (20-100% ACN with 0.1 % TFA 6 min), m/z [M + H]⁺ C₁₂H₁₆FN₃O₂ requires: 269.3, found: 270.1 HPLC t_{R} (min) 3.71, 97% (20-100% ACN with 0.1 % TFA 10 min.) |
| (3S, 4R)-4- methoxytetrahydrofuran- 3-yl)amine | 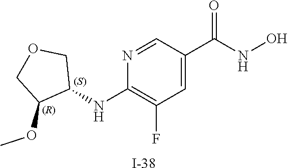 | ¹H NMR (400 MHz, MeOH-d4) δ 8.33 (s, 1 H), 7.60 (d, J = 11.98 Hz, 1 H), 4.60 (br, s, 1 H), 4.20-4.05 (m, 2 H), 4.00-3.90 (m, 1 H), 3.85-3.75 (m, 2 H), 3.48 (s, 3 H). LC-MS: t_{R} (min) 1.39 (20-100% ACN with 0.1 % TFA 6 min), m/z [M + H]⁺ C₁₁H₁₄FN₃O₂ requires: 271.3, found: 272.1 HPLC t_{R} (min) 2.97, 97% (20-100% ACN with 0.1 % TFA 10 min.) |
| 3,3- difluoro-1- (methoxymethyl) cyclobutan- 1-amine | 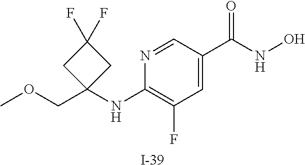 | ¹H NMR (400 MHz, MeOH-d4) δ 8.16 (s, 1 H), 7.48 (d, J = 12.2 Hz, 1 H), 4.52 (s, 2 H), 3.63 (s, 2 H), 3.26- 3.23 (m, 3 H), 2.82-2.72 (m, 4H). LC-MS: t_{R} (min) 3.00 (20-100% ACN with 0.1 % TFA 6 min), m/z [M + H]⁺ C₁₂H₁₄F3N₃O₃ requires: 305.3, found: 306.1 HPLC t_{R} (min) 5.02, 93% (20-100% ACN with 0.1 % TFA 10 min.) |
| 3-ethyloxetan-3- amine | 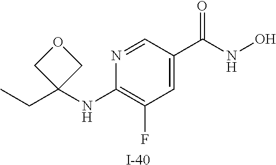 | ¹H NMR (400 MHz, MeOH-d4) δ 8.22 (s, 1 H), 7.59 (d, J = 11.98 Hz, 1 H), 4.80 (br, s, 2 H), 4.60 (br, s, 2 H), 2.22 (q, J = 6.93 Hz, 2 H), 0.92 (t, J = 6.95 Hz, 3 H). LC-MS: t_{R} (min) 1.10 (20-100% ACN with 0.1 % TFA 6 min), m/z [M + H]⁺ C₁₁H₁₃FN₃O₃ requires: 255.2, found: 256.1 HPLC t_{R} (min) 3.80, 99% (20-100% ACN with 0.1 % TFA 10 min.) |
| 1,1′-bi (cyclopropan)- 1-amine | 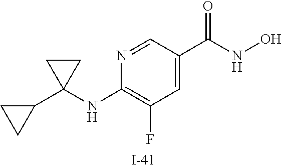 | ¹H NMR (400 MHz, MeOH-d4) δ 8.07 (s, 1 H), 7.32 (d, J = 11.98 Hz, 1 H), 4.40 (br, s, 1 H), 3.08 (s, 1 H), 1.35-1.25 (m, 1 H), 0.48 (d, J = 5.38 Hz, 4 H), 0.38- 0.42 (m, 2 H), 0.01 (s, 2 H). LC-MS: t_{R} (min) 1.20 (20-100% ACN with 0.1 % TFA 6 min), m/z [M + H]⁺ C₁₁H₁₃FN₃O₃ requires: 251.3, found: 252.1 HPLC t_{R} (min) 3.17, 96% (20-100% ACN with 0.1 % TFA 10 min.) |
| (S)-1- methyl-2- oxopyrrolidin-3- amine | 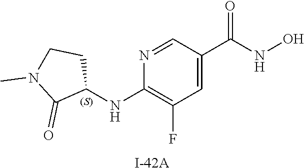 | ¹H NMR (400 MHz, DMSO-d6) δ 11.05 (br, s, 1 H), 9.00 (br, s, 1 H), 8.50 (br, s, 1 H), 7.65 (d, J = 12.0 Hz, 1 H), 7.45 (d, J = 8.80 Hz, 1 H), 4.90-4.80 (m, 1 H), 3.45-3.40 (m, 2 H), 2.76 (s, 3 H), 2.30-2.20 (m, 1 H), 1.90-2.00 (m, 1 H). LC-MS: t_{R} (min) 1.10 (20-100% ACN with 0.1 % TFA 6 min), m/z [M + H]⁺ C₁₁H₁₃FN₃O₃ requires: 268.3, found: 269.1 HPLC t_{R} (min) 1.54, 96% (20-100% ACN with 0.1 % TFA 10 min.) |
| (S)-2- oxopyrrolidin-3- amine | 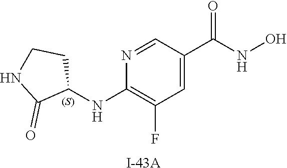 | ¹H NMR (400 MHz, DMSO-d6) δ 11.05 (br, s, 1 H), 8.95 (br, s, 1 H), 8.25 (s, 1 H), 7.83 (s, 1 H), 7.65 (d, J = 12.0 Hz, 1 H), 7.45 (d, J = 8.80 Hz, 1 H), 4.70-4.85 (m, 1 H), 3.23 (d, J = 6.34 Hz, 2 H), 2.40-2.30 (m, 1 H), 2.15-2.05 (m, 1 H). LC-MS: tR (min) 1.10 (20-100% ACN with 0.1 % TFA 6 min), m/z [M + H]⁺ C₁₂H₁₆FN₃O₃ requires: 254.2, found: 255.1 HPLC t_{R} (min) 1.71, 96% (20-100% ACN with 0.1 % TFA 10 min.) |
| (R)-1- methyl-2- oxopyrrolidin-3- amine | 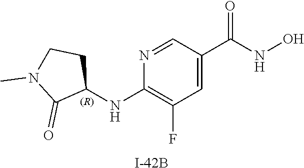 | ¹H NMR (400 MHz, DMSO-d6) δ 11.05 (br, s, 1 H), 8.99 (br, s, 1 H), 8.25 (s, 1 H), 7.65 (d, J = 12.2 Hz, 1 H), 7.42 (d, d, J = 8.07 Hz, 1 H), 4.82 (d, J = 9.05 Hz, 1 H), 3.34-3.27 (m, 2 H), 2.76 (s, 3 H), 2.40-2.26 (m, 1 H), 2.03-1.92 (m, 1 H). LC-MS: t_{R} (min) 1.20 (20-100% ACN with 0.1 % TFA 6 min), m/z [M + H]⁺ C₁₂H₁₆FN₃O₃ requires: 268.3, found: 269.1 HPLC t_{R} (min) 1.75, 99% (20-100% ACN with 0.1 % TFA 10 min.) |
| (R)-2- oxopyrrolidin-3- amine | 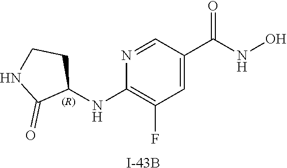 | ¹H NMR (400 MHz, DMSO-d6) δ 11.0 (br, s, 1 H), 9.05 (br, s, 1 H), 8.45 (s, 1 H), 7.84 (s, 1 H), 7.65 (d, J = 12.0 Hz, 1 H), 7.34 (d, J = 8.80 Hz, 1 H), 4.77 (d, J = 9.29 Hz, 1 H), 3.18-3.28 (m, 2 H), 2.30-2.40 (m, 1 H), 2.15-2.05 (m, 1 H). LC-MS: t_{R} (min) 1.15 (20-100% ACN with 0.1 % TFA 6 min), m/z [M + H]⁺ C₁₂H₁₆FN₃O₃ requires: 254.2, found: 255.1 HPLC t_{R} (min) 1.55, 93% (20-100% ACN with 0.1 % TFA 10 min.) |
| 1-(2- methoxyethyl) cyclopropyl) amine | 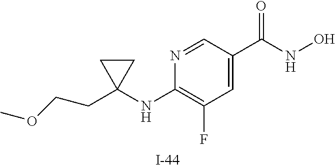 | ¹H NMR (400 MHz, MeOH-d4) δ 8.32 (s, 1 H), 7.57 (d, J = 11.98 Hz, 1 H), 3.55 (t, J = 6.85, 2 H), 3.30 (s, 3 H), 1.96 (t, J = 6.85 Hz, 2 H), 0.80 (s, 4 H). LC-MS: t_{R} (min) 1.20 (20-100% ACN with 0.1 % TFA 6 min), m/z [M + H]⁺ C₁₂H₁₆FN₃O₃ requires: 269.3, found: 270.1 HPLC t_{R} (min) 2.07, 99% (20-100% ACN with 0.1 % TFA 10 min.) |
| 3- phenyloxetan-3- amine | 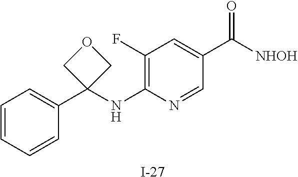 | ¹H NMR (400 MHz, DMSO-d6) δ 10.95 (br s, 1 H), 8.97 (s, 1 H), 8.38 (m, 1 H), 8.02 (m, 1 H), 7.69 (m, 1 H), 7.53 (m, 2 H), 7.34 (m, 2 H), 7.24 (m, 1 H), 4.99 (m, 2 H), 4.78 (m, 2 H). LC-MS: m/z [M + H]⁺ C₁₅H₁₄FN₃O₃ requires: 303.2, found: 304.1 HPLC t_{R} (min) 4.81, 95% (10-100% ACN with 0.1 % TFA 10 min.) |
| 3- (pyridin- 2- yl)oxetan- 3-amine | 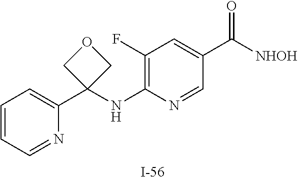 | ¹H NMR (400 MHz, DMSO-d6) δ 10.97 (br s, 1 H), 8.97 (s, 1 H), 8.63 (m, 1 H), 8.46 (m, 1 H), 7.99 (m, 1 H), 7.71-7.66 (m, 2 H), 7.31-7.15 (m, 2 H), 5.01 (m, 2 H), 4.87 (m, 2 H). LC-MS: m/z [M + H]⁺ C₁₄H₁₃FN₄O₃ requires: 304.2, found: 305.1 HPLC t_{R} (min) 1.27, 93% (10-100% ACN with 0.1 % TFA 10 min.) |
| 1H- pyrrolo[2, 3-b]pyridine | 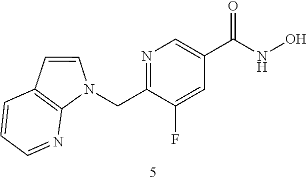 | ¹H NMR (400 MHz, DMSO-d6) δ 11.41 (br s, 1 H), 9.31 (s, 1 H), 8.57 (m, 1 H), 8.18 (m, 1 H), 7.98 (m, 2 H), 7.60 (m, 1 H), 7.08 (m, 1 H), 6.52 (m, 1 H), 5.71 (s, 2 H). LC-MS: m/z [M + H]⁺ C₁₄H₁₁FN₄O₂ requires: 286.2, found: 287.1 HPLC t_{R} (min) 3.21, 98% (20-100% ACN with 0.1 % TFA 10 min.) |
| 2- (difluoromethyl)- 1H- benzo[d]imidazole | 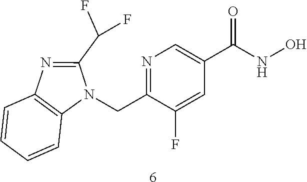 | ¹H NMR (400 MHz, DMSO-d6) δ 11.41 (br s, 1 H), 9.32 (s, 1 H), 8.51 (s, 1 H), 8.01 (m, 1 H), 7.63 (m, 1 H), 7.62 (m, 1 H), 7.53-7.27 (m, 3 H), 5.92 (s, 2 H). LC-MS: m/z [M + H]⁺ C₁₅H₁₁F₃N₄O₂ requires: 336.2.1, found: 337.1 HPLC t_{R} (min) 4.87, 97% (10-100% ACN with 0.1 % TFA 10 min.) |
| 2,2-dimethyl- 2H- pyrido[3, 2-b][1,4] oxazin- 3(4H)- one | 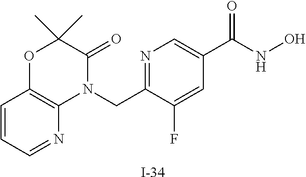 | ¹H NMR (400 MHz, DMSO-d6) δ 11.37 (br s, 1 H), 9.30 (s, 1 H), 8.52 (s, 1 H), 7.97 (m, 1 H), 7.88 (m, 1 H), 7.44 (m, 1 H), 7.03 (m, 1 H), 5.42 (s, 2 H), 1.51 (s, 6 H). LC-MS: m/z [M + H]⁺ C₁₆H₁₅FN₄O₄ requires: 346.3, found: 347.1 HPLC t_{R} (min) 5.04, 100% (10-100% ACN with 0.1 % TFA 10 min.) |
| 3-methyl-3,4- dihydroquinazolin- 2(1H)-one | 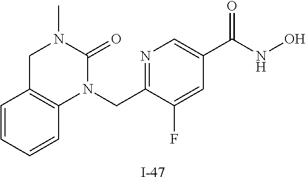 | ¹H NMR (400 MHz, DMSO-d6) δ 11.40 (br s, 1 H), 9.30 (s, 1 H), 8.55 (s, 1 H), 7.94 (m, 1 H), 7.15-7.11 (m, 2 H), 6.93 (m, 1 H), 6.74 (m, 1 H), 5.22 (s, 2 H), 4.40 (s, 2 H), 2.90 (s, 3 H). LC-MS: m/z [M + H]⁺ C₁₆H₁₅FN₄O₃ requires: 330.3, found: 331.1 HPLC t_{R} (min) 4.93, 100% (10-100% ACN with 0.1 % TFA 10 min.) |
| 3,4- dihydro- 2H- thieno[3, 2- b]indole 1,1- dioxide | 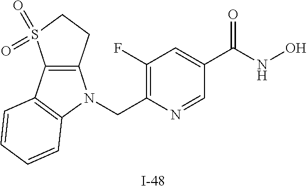 | ¹H NMR (400 MHz, DMSO-d6) δ 8.62 (s, 1 H), 8.02 (m, 1 H), 7.56 (m, 2 H), 7.27 (m, 2 H), 5.68 (s, 2 H), 3.94 (m, 2 H), 3.46 (m, 2 H). LC-MS: m/z [M + H]⁺ C₁₇H₁₄FN₃O₄S requires: 375.3, found: 376.1 HPLC t_{R} (min) 4.85, 96% (10-100% ACN with 0.1 % TFA 10 min.) |
| 2-(trifluoro methyl)-1H- benzo[d]imidazole | 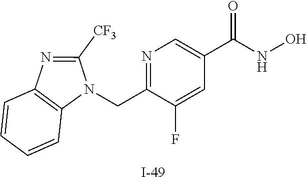 | ¹H NMR (400 MHz, DMSO-d6) δ 11.41 (br s, 1 H), 9.33 (s, 1 H), 8.49 (s, 1 H), 8.04 (m, 1 H), 7.84 (m, 1 H), 7.72 (m, 1 H), 7.44 (m, 2 H), 5.95 (s, 2 H). LC-MS: m/z [M + H]⁺ C₁₅H₁₀F₄N₄O₂ requires: 354.2, found: 355.1 HPLC t_{R} (min) 4.94, 98% (10-100% ACN with 0.1 % TFA 10 min.) |
| 2-methyl- 1H- pyrrolo[2,3- b]pyridine | 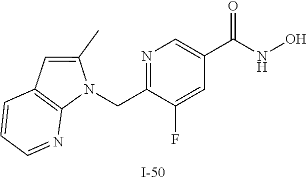 | ¹H NMR (400 MHz, DMSO-d6) δ 11.39 (br s, 1 H), 9.31 (s, 1 H), 8.51 (s, 1 H), 8.06 (m, 1 H), 7.97 (m, 1 H), 7.84 (m, 1 H), 7.03 (m, 1 H), 6.29 (s, 1 H), 5.69 (s, 2 H), 2.40 (s, 3 H). LC-MS: m/z [M + H]⁺ C₁₅H₁₃FN₄O₂ requires: 300.2, found: 301.1 HPLC t_{R} (min) 3.83, 100% (20-100% ACN with 0.1 % TFA 10 min.) |
| 2,3- dimethyl- 1H- pyrrolo[2,3- b]pyridine | 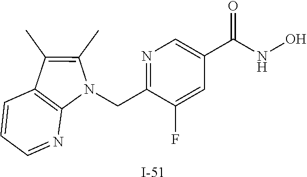 | ¹H NMR (400 MHz, DMSO-d6) δ 11.39 (br s, 1 H), 9.31 (s, 1 H), 8.51 (s, 1 H), 8.05 (m, 1 H), 7.95 (m, 1 H), 7.81 (m, 1 H), 7.01 (m, 1 H), 5.67 (s, 2 H), 2.31 (s, 3 H), 2.21 (s, 3 H). LC-MS: m/z [M + H]⁺ C₁₆H₁₅FN₄O₂ requires: 314.3, found: 315.1 HPLC t_{R} (min) 4.17, 100% (20-100% ACN with 0.1 % TFA 10 min.) |
| oxazolo [4,5- b]pyridin- 2(3H)- one | 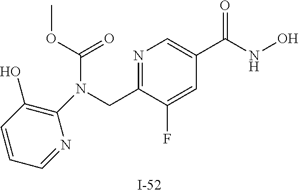 | ¹H NMR (400 MHz, CD₃OD-d4) δ 8.70 (s, 1 H), 7.98 (m, 2 H), 7.47 (m, 1 H), 7.38 (m, 1 H), 5.06 (s, 2 H), 3.64 (s, 3 H). LC-MS: m/z [M + H]⁺ C₁₄H₁₃FN₄O₅ requires: 336.2, found: 337.1 HPLC t_{R} (min) 4.18, 96% (10-100% ACN with 0.1 % TFA 10 min.) |
| 2-methyl- 3H-imidazo[4,5- b]pyridine | 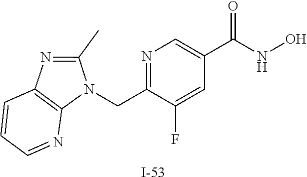 | ¹H NMR (400 MHz, DMSO-d6) δ 11.30 (br s, 1 H), 9.27 (s, 1 H), 8.47 (s, 1 H), 8.12 (m, 1 H), 7.95 (m, 1 H), 7.88 (m, 1 H), 7.15 (m, 1 H), 5.68 (s, 2 H), 2.44 (s, 3 H). LC-MS: m/z [M + H]⁺ C₁₄H₁₂FN₅O₂ requires: 301.2, found: 302.1 HPLC t_{R} (min) 3.60, 98% (10-100% ACN with 0.1 % TFA 10 min.) |
| 4-(1H- benzo[d]imidazol- 2-yl)morpholine | 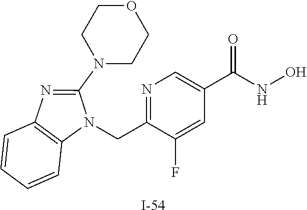 | ¹H NMR (400 MHz, DMSO-d6) δ 11.45 (br s, 1 H), 9.34 (s, 1 H), 8.59 (s, 1 H), 8.03 (m, 1 H), 7.45 (m, 1 H), 7.15-7.03 (m, 3 H), 5.55 (s, 2 H), 3.67 (m, 4 H), 3.14 (m, 4 H). LC-MS: m/z [M + H]⁺ C₁₈H₁₈FN₅O₃ requires: 371.3, found: 372.1 HPLC t_{R} (min) 3.66, 95% (10-100% ACN with 0.1 % TFA 10 min.) |
| 2-methyl- 2,3,4,5- tetrahydro- 1H-pyrido[4, 3-b]indole | 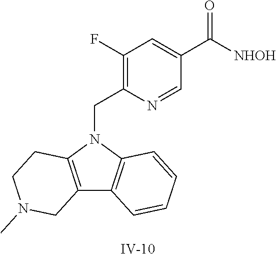 | ¹H NMR (400 MHz, DMSO-d6) δ 11.40 (br s, 1 H), 9.36 (s, 1 H), 8.61 (s, 1 H), 7.95 (m, 1 H), 7.35 (m, 2 H), 7.03-6.94 (m, 2 H), 5.50 (s, 2 H), 3.52 (s, 2 H), 2.83 (m, 2 H), 2.72 (m, 2 H), 2.42 (s, 3 H). LC-MS: m/z [M + H]⁺ C₁₉H₁₉FN₄O₂ requires: 354.3, found: 355.2 HPLC t_{R} (min) 4.58, 99% (10-100% ACN with 0.1 % TFA 10 min.) |
| 1,3,4,5- tetrahydr opyrano[4,3- b]indole | 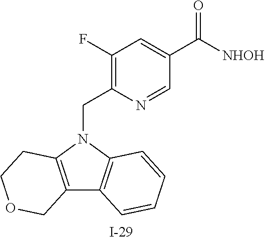 | ¹H NMR (400 MHz, DMSO-d6) δ 11.41 (br s, 1 H), 9.35 (s, 1 H), 8.61 (s, 1 H), 8.01 (m, 1 H), 7.40-7.32 (m, 2 H), 7.06-6.95 (m, 2 H), 5.54 (s, 2 H), 4.78 (s, 2 H), 3.95 (m, 2 H), 2.84 (m, 2 H). LC-MS: m/z [M + H]⁺ C₁₈H₁₆FN₃O₃ requires: 341.3, found: 342.1 HPLC t_{R} (min) 4.99, 94% (10-100% ACN with 0.1 % TFA 10 min.) |

| Compound | Characterization Data |
|---|---|
| 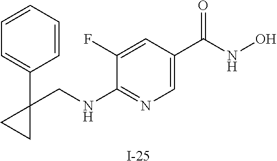 | ¹H NMR (400 MHz, Solvent) δ ppm 8.18 (s, 1 H) 7.50 (br d, J = 11.98 Hz, 1 H) 7.31 − 7.38 (m, 2 H) 7.23 (br t, J = 7.46 Hz, 2 H) 7.10 − 7.17 (m, 1 H) 3.74 (s, 2 H) 0.93 − 0.99 (m, 2 H) 0.80 − 0.86 (m, 2 H) |
| 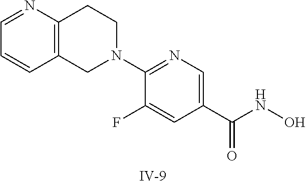 [SMILES: O=C(NO)c1cnc(N2CCc3ncccc3C2)c(F)c1 ; "IV-9"] | ¹H NMR (400 MHz, Solvent) δ ppm 8.39 (s, 1 H) 8.35 (d, J = 4.65 Hz, 1 H) 7.64 − 7.76 (m, 2 H) 7.27 (dd, J = 7.58, 5.14 Hz, 1 H) 4.86 (s, 2 H) 4.03 (t, J = 5.87 Hz, 2 H) 3.10 (t, J = 5.87 Hz, 2 H) |
| 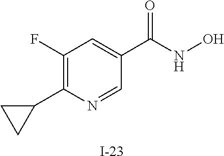 | ¹H NMR (400 MHz, METHANOL-d4) δ ppm 8.57 (s, 1 H) 7.77 (d, J = 10.27 Hz, 1 H) 2.31 − 2.46 (m, 1 H) 1.08 − 1.16 (m, 4 H) LCMS R_{T} = 2.79 min, m/z = 197.1 [M + H]⁺. |
| 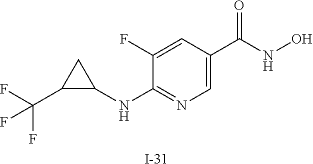 | ¹H NMR (400 MHz, METHANOL-d4) δ ppm 8.34 (s, 1 H) 7.60 (br d, J = 11.49 Hz, 1 H) 3.22 − 3.28 (m, 1 H) 1.82 − 1.95 (m, 1 H) 1.21 − 1.31 (m, 1 H) 1.16 (br dd, J = 10.03, 4.89 Hz, 1 H) |
| 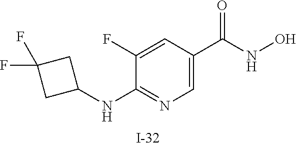 | ¹H NMR (400 MHz, METHANOL-d4) δ ppm 8.30 (s, 1 H) 7.61 (br d, J = 11.74 Hz, 1 H) 4.32 − 4.47 (m, 1 H) 2.94 − 3.12 (m, 2 H) 2.54 − 2.76 (m, 2 H) |
| 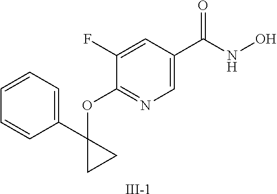 | ¹H NMR (400 MHz, METHANOL-d4) δ ppm 8.21 (s, 1 H) 7.82 (br d, J = 10.27 Hz, 1 H) 7.25 − 7.32 (m, 4 H) 7.15 − 7.23 (m, 1 H) 1.44 − 1.50 (m, 2 H) 1.36 − 1.43 (m, 2 H) |

### REFERENCES

Arber, S., Hunter, J. J., Ross, J., Hongo, M., Sansig, G., Borg, J., Perriard, J. C., Chien, K. R., Caroni, P., 1997. MLP-Deficient Mice Exhibit a Disruption of Cardiac Cytoarchitectural Organization, Dilated Cardiomyopathy, and Heart Failure. Cell 88, 393-403. https://doi.org/10.1016/S0092-8674(00)81878-4
Bacon, T., Seiler, C., Wolny, M., Hughes, R., Watson, P., Schwabe, J., Grigg, R., Peckham, M., 2015. Histone deacetylase 3 indirectly modulates tubulin acetylation. Biochem. J. 472, 367-377. https://doi.org/10.1042/BJ20150660
Buikema, J. W., Lee, S., Goodyer, W. R., Maas, R. G., Chirikian, O., Li, G., Miao, Y., Paige, S. L., Lee, D., Wu, H., Paik, D. T., Rhee, S., Tian, L., Galdos, F. X., Puluca, N., Beyersdorf, B., Hu, J., Beck, A., Venkamatran, S., Swami, S., Wijnker, P., Schuldt, M., Dorsch, L. M., van Mil, A., Red-Horse, K., Wu, J. Y., Geisen, C., Hesse, M., Serpooshan, V., Jovinge, S., Fleischmann, B. K., Doevendans, P. A., van der Velden, J., Garcia, K. C., Wu, J. C., Sluijter, J. P. G., Wu, S. M., 2020. Wnt Activation and Reduced Cell-Cell Contact Synergistically Induce Massive Expansion of Functional Human iPSC-Derived Cardiomyocytes. Cell Stem Cell 27, 50-63.e5. https://doi.org/10.1016/j.stem.2020.06.001
Cao, D. J., Wang, Z. V., Battiprolu, P. K., Jiang, N., Morales, C. R., Kong, Y., Rothermel, B. A., Gillette, T. G., Hill, J. A., 2011. Histone deacetylase (HDAC) inhibitors attenuate cardiac hypertrophy by suppressing autophagy. Proc. Natl. Acad. Sci. 108, 4123-4128. https://doi.org/10.1073/pnas.1015081108
Chami, N., Tadros, R., Lemarbre, F., Lo, K. S., Beaudoin, M., Robb, L., Labuda, D., Tardif, J. C., Racine, N., Talajic, M., Lettre, G., 2014. Nonsense mutations in BAG3 are associated with early-onset dilated cardiomyopathy in French Canadians. Can. J. Cardiol. 30, 1655-1661. https://doi.org/10.1016/j.cjca.2014.09.030
Chen, C. Y., Caporizzo, M. A., Bedi, K., Vite, A., Bogush, A. I., Robison, P., Heffler, J. G., Salomon, A. K., Kelly, N. A., Babu, A., Morley, M. P., Margulies, K. B., Prosser, B. L., 2018. Suppression of detyrosinated microtubules improves cardiomyocyte function in human heart failure. Nat. Med. 1. https://doi.org/10.1038/s41591-018-0046-2
Cleland, J. G. F., Lyon, A. R., McDonagh, T., McMurray, J. J. V., 2020. The year in cardiology: heart failure. Eur. Heart J. 41, 1232-1248. https://doi.org/10.1093/eurheartj/ehz949
Demos-Davies, K. M., Ferguson, B. S., Cavasin, M. A., Mahaffey, J. H., Williams, S. M., Spiltoir, J. I., Schuetze, K. B., Horn, T. R., Chen, B., Ferrara, C., Scellini, B., Piroddi, N., Tesi, C., Poggesi, C., Jeong, M. Y., McKinsey, T. A., 2014. HDAC6 contributes to pathological responses of heart and skeletal muscle to chronic angiotensin-II signaling. Am. J. Physiol.—Heart Circ. Physiol. 307, H252-H258. https://doi.org/10.1152/ajpheart.00149.2014
Dominguez, F., Cuenca, S., Bilińska, Z., Toro, Rocio, Villard, E., Barriales-Villa, R., Ochoa, J. P., Asselbergs, F., Sammani, A., Franaszczyk, M., Akhtar, M., Coronado-Albi, M. J., Rangel-Sousa, D., Rodriguez-Palomares, J. F., Jiménez-Jáimez, J., Garcia-Pinilla, José Manuel, Ripoll-Vera, T., Mogollón-Jimenez, M. V., Fontalba-Romero, A., Garcia-Medina, D., Palomino-Doza, J., de Gonzalo-Calvo, D., Cicerchia, M., Salazar-Mendiguchia, J., Salas, C., Pankuweit, S., Hey, T. M., Mogensen, J., Barton, P. J., Charron, P., Elliott, P., Garcia-Pavia, P., Eiskjær, H., Barriales, R., Fernández Fernández, X., Cicerchia, M., Monserrat, L., Ochoa, J. P., Salazar-Mendiguchia, J., Mogollón, M. V., Ripoll, T., Charron, P., Richard, P., Villard, E., Palomino Doza, J., Fontalba, A., Alonso-Pulpón, L., Cobo-Marcos, M., Domínguez, F., Garcia-Pavia, P., Gómez-Bueno, M., Gonzilez-López, E., Hernández-Hernández, A., Hernández-Perez, F. J., López-Sainz, Á., Restrepo-Córdoba, A., Segovia-Cubero, J., Toro, Rocio, de Gonzalo-Calvo, D., Rosa Longobardo, F., Limeres, J., Rodriguez-Palomares, J. F., Garcia-Pinilla, Jose Manuel, López-Garrido, M. A., Jiménez-Jaimez, J., Garcia-Medina, D., Rangel Sousa, D., Peña, M. L., Mogensen, J., Morris-Hey, T., Barton, P. J., Cook, S. A., Midwinter, W., Roberts, A. M., Ware, J. S., Walsh, R., Akhtar, M., Elliott, P. M., Rocha-Lopes, L., Savvatis, K., Syrris, P., Michalak, E., Ploski, R., Sobieszczanska-Malek, M., Bilinska, Z., Pankuweit, S., Asselbergs, F., Baas, A., Dooijes, D., Sammani, A., 2018. Dilated Cardiomyopathy Due to BLC2-Associated Athanogene 3 (BAG3) Mutations. J. Am. Coll. Cardiol. 72, 2471-2481. https://doi.org/10.1016/j.jacc.2018.08.2181
Everly, M. J., 2008. Cardiac transplantation in the United States: an analysis of the UNOS registry. Clin. Transpl. 35-43.
Fang, X., Bogomolovas, J., Wu, T., Zhang, W., Liu, C., Veevers, J., Stroud, M. J., Zhang, Z., Ma, X., Mu, Y., Lao, D. H., Dalton, N. D., Gu, Y., Wang, C., Wang, M., Liang, Y., Lange, S., Ouyang, K., Peterson, K. L., Evans, S. M., Chen, J., 2017. Loss-of-function mutations in co-chaperone BAG3 destabilize small HSPs and cause cardiomyopathy. J. Clin. Invest. 127. https://doi.org/10.1172/JC194310
Feldman, A. M., Begay, R. L., Knezevic, T., Myers, V. D., Slavov, D. B., Zhu, W., Gowan, K., Graw, S. L., Jones, K. L., Tilley, D. G., Coleman, R. C., Walinsky, P., Cheung, J. Y., Mestroni, L., Khalili, K., Taylor, M. R. G., 2014. Decreased Levels of BAG3 in a Family With a Rare Variant and in Idiopathic Dilated Cardiomyopathy. J. Cell. Physiol. 229, 1697-1702. https://doi.org/10.1002/jcp.24615
Franceschelli, S., Rosati, A., Lerose, R., De Nicola, S., Turco, M. C., Pascale, M., 2008. Bag3 gene expression is regulated by heat shock factor 1. J. Cell. Physiol. 215, 575-577. https://doi.org/10.1002/jcp.21397
Gallinari, P., Marco, S. D., Jones, P., Pallaoro, M., Steinkühler, C., 2007. HDACs, histone deacetylation and gene transcription: from molecular biology to cancer therapeutics. Cell Res. 17, 195-211. https://doi.org/10.1038/sj.cr.7310149
Gamerdinger, M., Kaya, A. M., Wolfrum, U., Clement, A. M., Behl, C., 2011. BAG3 mediates chaperone-based aggresome-targeting and selective autophagy of misfolded proteins. EMBO Rep. 12, 149-156. https://doi.org/10.1038/embor.2010.203
Gao, X., Shen, L., Li, X., Liu, J., 2019. Efficacy and toxicity of histone deacetylase inhibitors in relapsed/refractory multiple myeloma: Systematic review and meta-analysis of clinical trials. Exp. Ther. Med. 18, 1057-1068. https://doi.org/10.3892/etm.2019.7704
Glozak, M. A., Seto, E., 2007. Histone deacetylases and cancer. Oncogene 26, 5420-5432. https://doi.org/10.1038/sj.onc.1210610
Graff, J., Tsai, L. H., 2013. The potential of HDAC inhibitors as cognitive enhancers. Annu. Rev. Pharmacol. Toxicol. 53, 311-330. https://doi.org/10.1146/annurev-pharmtox-011112-140216
Haas, J., Frese, K. S., Peil, B., Kloos, W., Keller, A., Nietsch, R., Feng, Z., Müller, S., Kayvanpour, E., Vogel, B., Sedaghat-Hamedani, F., Lim, W. K., Zhao, X., Fradkin, D., Kohler, D., Fischer, S., Franke, J., Marquart, S., Barb, I., Li, D. T., Amr, A., Ehlermann, P., Mereles, D., Weis, T., Hassel, S., Kremer, A., King, V., Wirsz, E., Isnard, R., Komajda, M., Serio, A., Grasso, M., Syrris, P., Wicks, E., Plagnol, V., Lopes, L., Gadgaard, T., Eiskjær, H., Jorgensen, M., Garcia-Giustiniani, D., Ortiz-Genga, M., Crespo-Leiro, M. G., Deprez, R. H. L. D., Christiaans, I., Rijsingen, I. A. van, Wilde, A. A., Waldenstrom, A., Bolognesi, M., Bellazzi, R., Mörner, S., Bermejo, J. L., Monserrat, L., Villard, E., Mogensen, J., Pinto, Y. M., Charron, P., Elliott, P., Arbustini, E., Katus, H. A., Meder, B., 2015. Atlas of the clinical genetics of human dilated cardiomyopathy. Eur. Heart J. 36, 1123-1135. https://doi.org/10.1093/eurheartj/ehu301
Haberland, M., Montgomery, R. L., Olson, E. N., 2009. The many roles of histone deacetylases in development and physiology: implications for disease and therapy. Nat. Rev. Genet. 10, 32-42. https://doi.org/10.1038/nrg2485
Heidersbach, A., Saxby, C., Carver-Moore, K., Huang, Y., Ang, Y. S., de Jong, P. J., Ivey, K. N., Srivastava, D., 2013. MicroRNA-1 regulates sarcomere formation and suppresses smooth muscle gene expression in the mammalian heart. ELife 2. https://doi.org/10.7554/eLife.01323
Homma, S., Iwasaki, M., Shelton, G. D., Engvall, E., Reed, J. C., Takayama, S., 2006. BAG3 Deficiency Results in Fulminant Myopathy and Early Lethality. Am. J. Pathol. 169, 761-773. https://doi.org/10.2353/ajpath.2006.060250
Hubbert, C., Guardiola, A., Shao, R., Kawaguchi, Y., Ito, A., Nixon, A., Yoshida, M., Wang, X. F., Yao, T. P., 2002. HDAC6 is a microtubule-associated deacetylase. Nature 417, 455-458. https://doi.org/10.1038/417455a
Jeong, M. Y., Lin, Y. H., Wennersten, S. A., Demos-Davies, K. M., Cavasin, M. A., Mahaffey, J. H., Monzani, V., Saripalli, C., Mascagni, P., Reece, T. B., Ambardekar, A. V., Granzier, H. L., Dinarello, C. A., McKinsey, T. A., 2018. Histone deacetylase activity governs diastolic dysfunction through a nongenomic mechanism. Sci. Transl. Med. 10, eaao0144. https://doi.org/10.1126/scitranslmed.aao0144
Joshi, P., Greco, T. M., Guise, A. J., Luo, Y., Yu, F., Nesvizhskii, A. I., Cristea, I. M., 2013. The functional interactome landscape of the human histone deacetylase family. Mol. Syst. Biol. 9, 672. https://doi.org/10.1038/msb.2013.26
Judge, L. M., Perez-Bermejo, J. A., Truong, A., Ribeiro, A. J. S., Yoo, J. C., Jensen, C. L., Mandegar, M. A., Huebsch, N., Kaake, R. M., So, P. L., Srivastava, D., Pruitt, B. L., Krogan, N. J., Conklin, B. R., 2017. A BAG3 chaperone complex maintains cardiomyocyte function during proteotoxic stress. JCI Insight 2. https://doi.org/10.1172/jci.insight.94623
Knoll, R., Kostin, S., Klede, S., Savvatis, K., Klinge, L., Stehle, I., Gunkel, S., Kotter, S., Babicz, K., Sohns, M., Miocic, S., Didie, M., Knöll, G., Zimmermann, W. H., Thelen, P., Bickeböller, H., Maier, L. S., Schaper, W., Schaper, J., Kraft, T., Tschöpe, C., Linke, W. A., Chien, K. R., 2010. A Common MLP (Muscle LIM Protein) Variant Is Associated With Cardiomyopathy. Circ. Res. 106, 695-704. https://doi.org/10.1161/CIRCRESAHA.109.206243
LeCun, Y., Bengio, Y., Hinton, G., 2015. Deep learning. Nature 521, 436-444. https://doi.org/10.1038/nature14539
Leoni, F., Fossati, G., Lewis, E. C., Lee, J. K., Porro, G., Pagani, P., Modena, D., Moras, M. L., Pozzi, P., Reznikov, L. L., Siegmund, B., Fantuzzi, G., Dinarello, C. A., Mascagni, P., 2005. The histone deacetylase inhibitor ITF2357 reduces production of pro-inflammatory cytokines in vitro and systemic inflammation in vivo. Mol. Med. Camb. Mass 11, 1-15. https://doi.org/10.2119/2006-00005. Dinarello
Lian, X., Hsiao, C., Wilson, G., Zhu, K., Hazeltine, L. B., Azarin, S. M., Raval, K. K., Zhang, J., Kamp, T. J., Palecek, S. P., 2012. Robust cardiomyocyte differentiation from human pluripotent stem cells via temporal modulation of canonical Wnt signaling. Proc. Natl. Acad. Sci. 201200250. https://doi.org/10.1073/pnas.1200250109
Ludwig, T. E., Bergendahl, V., Levenstein, M. E., Yu, J., Probasco, M. D., Thomson, J. A., 2006. Feeder-independent culture of human embryonic stem cells. Nat. Methods 3, 637-646. https://doi.org/10.1038/nmeth902
Ma, Z., Huebsch, N., Koo, S., Mandegar, M. A., Siemons, B., Boggess, S., Conklin, B. R., Grigoropoulos, C. P., Healy, K. E., 2018. Contractile deficits in engineered cardiac microtissues as a result of MYBPC3 deficiency and mechanical overload. Nat. Biomed. Eng. https://doi.org/10.1038/s41551-018-0280-4
Maddah, M., Mandegar, M. A., Dame, K., Grafton, F., Loewke, K., Ribeiro, A. J. S., 2020. Quantifying drug-induced structural toxicity in hepatocytes and cardiomyocytes derived from hiPSCs using a deep learning method. J. Pharmacol. Toxicol. Methods 106895. https://doi.org/10.1016/j.vascn.2020.106895
Mariño, G., Niso-Santano, M., Baehrecke, E. H., Kroemer, G., 2014. Self-consumption: the interplay of autophagy and apoptosis. Nat. Rev. Mol. Cell Biol. 15, 81-94. https://doi.org/10.1038/nrm3735
McLendon, P. M., Ferguson, B. S., Osinska, H., Bhuiyan, M. S., James, J., McKinsey, T. A., Robbins, J., 2014. Tubulin hyperacetylation is adaptive in cardiac proteotoxicity by promoting autophagy. Proc. Natl. Acad. Sci. 111, E5178-E5186. https://doi.org/10.1073/pnas.1415589111
McNally, E. M., Golbus, J. R., Puckelwartz, M. J., 2013. Genetic mutations and mechanisms in dilated cardiomyopathy. J. Clin. Invest. 123, 19-26. https://doi.org/10.1172/JC162862
Milan, M., Pace, V., Maiullari, F., Chirivi, M., Baci, D., Maiullari, S., Madaro, L., Maccari, S., Stati, T., Marano, G., Frati, G., Puri, P. L., De Falco, E., Bearzi, C., Rizzi, R., 2018. Givinostat reduces adverse cardiac remodeling through regulating fibroblasts activation. Cell Death Dis. 9, 108. https://doi.org/10.1038/s41419-017-0174-5
Nagata, S., Marunouchi, T., Tanonaka, K., 2019. Histone Deacetylase Inhibitor SAHA Treatment Prevents the Development of Heart Failure after Myocardial Infarction via an Induction of Heat-Shock Proteins in Rats. Biol. Pharm. Bull. 42, 453-461. https://doi.org/10.1248/bpb.b18-00785
Norton, N., Li, D., Rieder, M. J., Siegfried, J. D., Rampersaud, E., Züchner, S., Mangos, S., Gonzalez-Quintana, J., Wang, L., McGee, S., Reiser, J., Martin, E., Nickerson, D. A., Hershberger, R. E., 2011. Genome-wide Studies of Copy Number Variation and Exome Sequencing Identify Rare Variants in BAG3 as a Cause of Dilated Cardiomyopathy. Am. J. Hum. Genet. 88, 273-282. https://doi.org/10.1016/j.ajhg.2011.01.016
Portran, D., Schaedel, L., Xu, Z., Théry, M., Nachury, M. V., 2017. Tubulin acetylation protects long-lived microtubules against mechanical ageing. Nat. Cell Biol. 19, 391-398. https://doi.org/10.1038/ncb3481
Rauch, J. N., Tse, E., Freilich, R., Mok, S. A., Makley, L. N., Southworth, D. R., Gestwicki, J. E., 2017. BAG3 Is a Modular, Scaffolding Protein that physically Links Heat Shock Protein 70 (Hsp70) to the Small Heat Shock Proteins. J. Mol. Biol. 429, 128-141. https://doi.org/10.1016/j.jmb.2016.11.013
Robison, P., Caporizzo, M. A., Ahmadzadeh, H., Bogush, A. I., Chen, C. Y., Margulies, K. B., Shenoy, V. B., Prosser, B. L., 2016. Detyrosinated microtubules buckle and bear load in contracting cardiomyocytes. Science 352, aaf0659. https://doi.org/10.1126/science.aaf0659
Ruparelia, A. A., Oorschot, V., Vaz, R., Ramm, G., Bryson-Richardson, R. J., 2014. Zebrafish models of BAG3 myofibrillar myopathy suggest a toxic gain of function leading to BAG3 insufficiency. Acta Neuropathol. (Berl.) 128, 821-833. https://doi.org/10.1007/s00401-014-1344-5
Schmittgen, T. D., Livak, K. J., 2008. Analyzing real-time PCR data by the comparative CT method. Nat. Protoc. 3, 1101-1108. https://doi.org/10.1038/nprot.2008.73
Stümer, E., Behl, C., 2017. The Role of the Multifunctional BAG3 Protein in Cellular Protein Quality Control and in Disease. Front. Mol. Neurosci. 10, 177. https://doi.org/10.3389/fnmol.2017.00177
Subramanian, S., Bates, S. E., Wright, J. J., Espinoza-Delgado, I., Piekarz, R. L., 2010. Clinical Toxicities of Histone Deacetylase Inhibitors. Pharmaceuticals 3, 2751-2767. https://doi.org/10.3390/ph3092751
Tarone, G., Brancaccio, M., 2014. Keep your heart in shape: molecular chaperone networks for treating heart disease. Cardiovasc. Res. 102, 346-361. https://doi.org/10.1093/cvr/cvu049
Villard, E., Perret, C., Gary, F., Proust, C., Dilanian, G., Hengstenberg, C., Ruppert, V., Arbustini, E., Wichter, T., Germain, M., Dubourg, O., Tavazzi, L., Aumont, M. C., DeGroote, P., Fauchier, L., Trochu, J. N., Gibelin, P., Aupetit, J. F., Stark, K., Erdmann, J., Hetzer, R., Roberts, A. M., Barton, P. J. R., Regitz-Zagrosek, V., Aslam, U., Duboscq-Bidot, L., Meyborg, M., Maisch, B., Madeira, H., Waldenström, A., Galve, E., Cleland, J. G., Dorent, R., Roizes, G., Zeller, T., Blankenberg, S., Goodall, A. H., Cook, S., Tregouet, D. A., Tiret, L., Isnard, R., Komajda, M., Charron, P., Cambien, F., 2011. A genome-wide association study identifies two loci associated with heart failure due to dilated cardiomyopathy. Eur. Heart J. 32, 1065-1076. https://doi.org/10.1093/eurheartj/ehr105
Vogl, D. T., Raje, N., Jagannath, S., Richardson, P., Hari, P., Orlowski, R., Supko, J. G., Tamang, D., Yang, M., Jones, S. S., Wheeler, C., Markelewicz, R. J., Lonial, S., 2017. Ricolinostat, the First Selective Histone Deacetylase 6 Inhibitor, in Combination with Bortezomib and Dexamethasone for Relapsed or Refractory Multiple Myeloma. Clin. Cancer Res. 23, 3307-3315. https://doi.org/10.1158/1078-0432.CCR-16-2526
Wallner et al., 2020. HDAC inhibition improves cardiopulmonary function in a feline model of diastolic dysfunction | Science Translational Medicine [WWW Document]. URL https://stm.sciencemag.org/content/12/525/eaay7205.full (accessed 1.17.20).
Watanabe, K., Ueno, M., Kamiya, D., Nishiyama, A., Matsumura, M., Wataya, T., Takahashi, J. B., Nishikawa, Satomi, Nishikawa, Shin-ichi, Muguruma, K., Sasai, Y., 2007. A ROCK inhibitor permits survival of dissociated human embryonic stem cells. Nat. Biotechnol. 25, 681-686. https://doi.org/10.1038/nbt1310
Xie Min, Kong Yongli, Tan Wei, May Herman, Battiprolu Pavan K., Pedrozo Zully, Wang Zhao V., Morales Cyndi, Luo Xiang, Cho Geoffrey, Jiang Nan, Jessen Michael E., Warner John J., Lavandero Sergio, Gillette Thomas G., Turer Aslan T., Hill Joseph A., 2014. Histone Deacetylase Inhibition Blunts Ischemia/Reperfusion Injury by Inducing Cardiomyocyte Autophagy. Circulation 129, 1139-1151. https://doi.org/10.1161/CIRCULATIONAHA.113.002416
Zhang, C. L., McKinsey, T. A., Chang, S., Antos, C. L., Hill, J. A., Olson, E. N., 2002. Class II Histone Deacetylases Act as Signal-Responsive Repressors of Cardiac Hypertrophy. Cell 110, 479-488. https://doi.org/10.1016/S0092-8674(02)00861-9
Zhang, Y., Kwon, S., Yamaguchi, T., Cubizolles, F., Rousseaux, S., Kneissel, M., Cao, C., Li, N., Cheng, H. L., Chua, K., Lombard, D., Mizeracki, A., Matthias, G., Alt, F. W., Khochbin, S., Matthias, P., 2008. Mice Lacking Histone Deacetylase 6 Have Hyperacetylated Tubulin but Are Viable and Develop Normally. Mol. Cell. Biol. 28, 1688-1701. https://doi.org/10.1128/MCB.01154-06

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

What is claimed is:

1. A method of treating or preventing dilated cardiomyopathy in a subject in need thereof, comprising administering to the subject a therapeutically effective amount of a HDAC6 inhibitor.

2. The method of claim 1, wherein the HDAC6 inhibitor is a compound according to Formula (I): 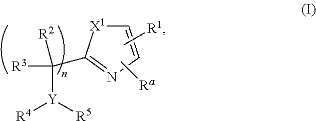
wherein
R¹ is selected from the group consisting of: 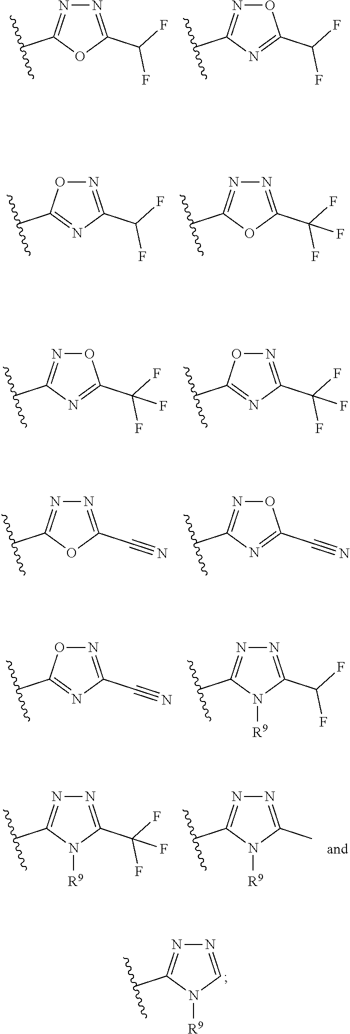
R^{a} is selected from the group consisting of H, halo, C₁₋₃ alkyl, cycloalkyl, haloalkyl, and alkoxy;
R² and R³ are independently selected from the group consisting of H, halogen, alkoxy, haloalkyl, aryl, heteroaryl, alkyl, and cycloalkyl each of which is optionally substituted, or R² and R³ together with the atom to which they are attached form a cycloalkyl or heterocyclyl;
R⁴ and R⁵ are independently selected from the group consisting of H, —(SO₂)R², —(SO₂)NR²R³, —(CO)R², —(CONR²R³), aryl, arylheteroaryl, alkylenearyl, heteroaryl, cycloalkyl, heterocyclyl, alkyl, haloalkyl, and alkoxy, each of which is optionally substituted, or R⁴ and R⁵ together with the atom to which they are attached form a cycloalkyl or heterocyclyl, each of which is optionally substituted;
R⁹ is selected from the group consisting of H, C₁-C₆ alkyl, haloalkyl, cycloalkyl and heterocyclyl;
X¹ is selected from the group consisting of S, O, NH and NR⁶, wherein R⁶ is selected from the group consisting of C₁-C₆ alkyl, alkoxy, haloalkyl, cycloalkyl and heterocyclyl;
Y is selected from the group consisting of CR², O, N, S, SO, and SO₂, wherein when Y is O, S, SO, or SO₂, R⁵ is not present and when R⁴ and R⁵ together with the atom to which they are attached form a cycloalkyl or heterocyclyl, Y is CR² or N; and
n is selected from 0, 1, and 2.

3. The method of claim 2, wherein the HDAC6 inhibitor is selected from the group consisting of

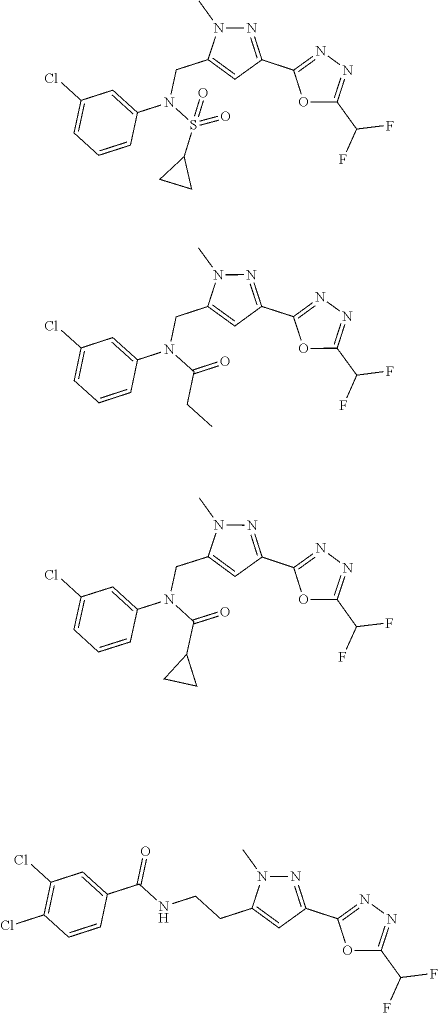 [SMILES: Cn1nc(-c2nnc(C(F)F)o2)cc1CN(c1cccc(Cl)c1)S(=O)(=O)C1CC1 ; CCC(=O)N(Cc1cc(-c2nnc(C(F)F)o2)nn1C)c1cccc(Cl)c1 ; Cn1nc(-c2nnc(C(F)F)o2)cc1CN(C(=O)C1CC1)c1cccc(Cl)c1 ; Cn1nc(-c2nnc(C(F)F)o2)cc1CCNC(=O)c1ccc(Cl)c(Cl)c1]

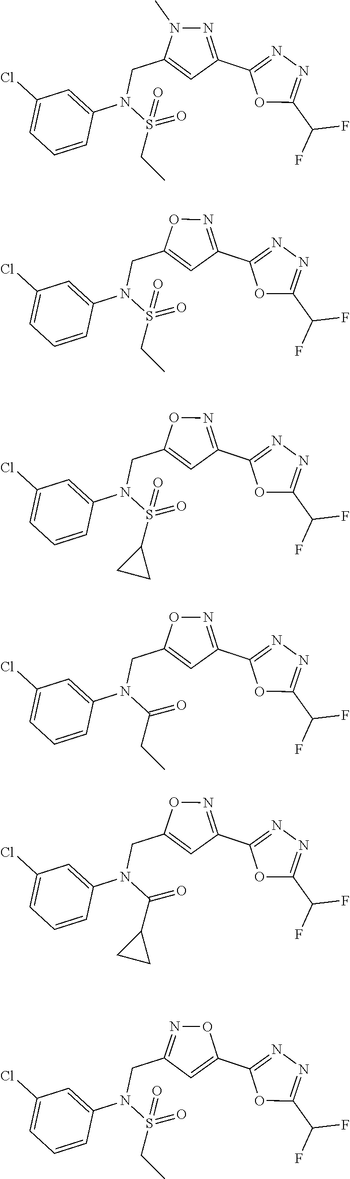

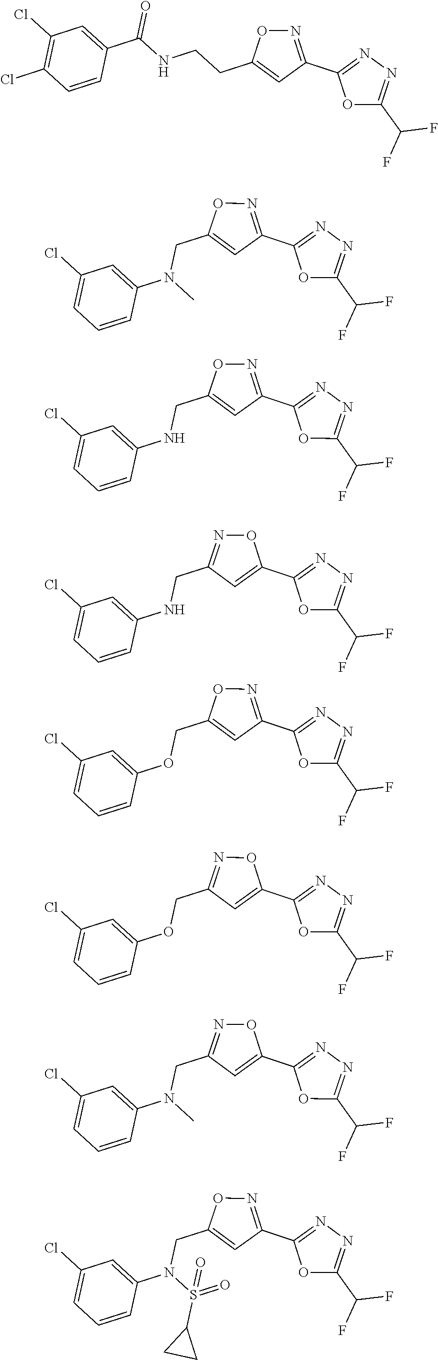

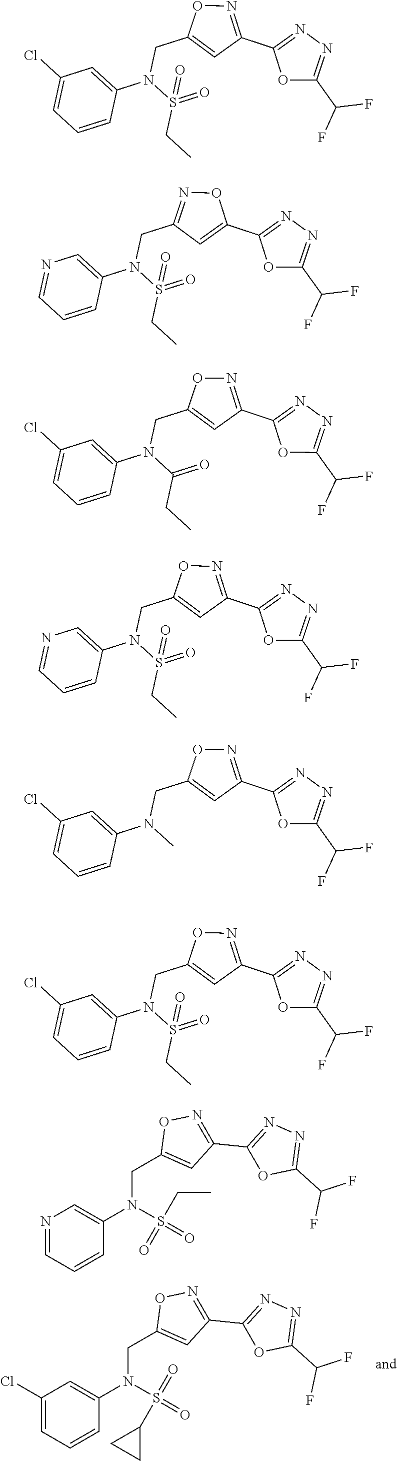

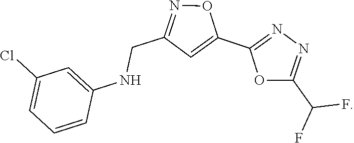 [SMILES: FC(F)c1nnc(-c2cc(CNc3cccc(Cl)c3)no2)o1]

4. The method of claim 3, wherein the HDAC6 inhibitor is 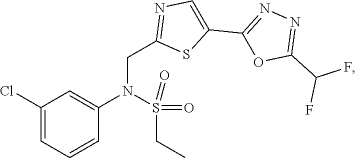 [SMILES: CCS(=O)(=O)N(Cc1ncc(-c2nnc(C(F)F)o2)s1)c1cccc(Cl)c1]
(TYA-018) or an analog thereof.

5. The method of claim 4, wherein the HDAC6 inhibitor is TYA-018.

6. The method of claim 1, wherein the HDAC6 inhibitor is a compound of Formula (II): 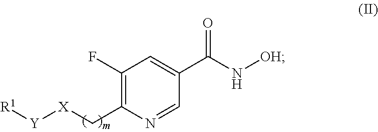
n is 0 or 1;
X is O, NR⁴, or CR⁴R^{4″};
Y is a bond, CR²R³ or S(O)₂;
R¹ is selected from the group consisting of H, amido, carbocyclyl, heterocyclyl, aryl, and heteroaryl;
R² and R³ are independently selected from the group consisting of H, halogen, alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or
R¹ and R² taken together with the carbon atom to which they are attached form a carbocyclyl or heterocyclyl; or
R² and R³ taken together with the carbon atom to which they are attached form a carbocyclyl or heterocyclyl; and
R⁴ and R^{4′} are each independently selected from the group consisting of H, alkyl, —CO₂-alkyl, carbocyclyl, heterocyclyl, aryl, heteroaryl, —(CH₂)-carbocyclyl, —(CH₂)-heterocyclyl, —(CH₂)-aryl, and —(CH₂)-heteroaryl; or
R⁴ and R^{4′} taken together with the carbon atom to which they are attached form a carbocyclyl or heterocyclyl;
wherein each alkyl, carbocyclyl, heterocyclyl, aryl, and heteroaryl is independently optionally substituted with one or more substituents selected from the group consisting of halogen, haloalkyl, oxo, hydroxy, alkoxy, —OCH₃, —CO₂CH₃, —C(O)NH(OH), —CH₃, morpholine, and —C(O)N-cyclopropyl.

7. The method of claim 1, wherein the HDAC6 inhibitor is CAY10603, tubacin, rocilinostat (ACY-1215), citarinostat (ACY-241), ACY-738, QTX-125, CKD-506, nexturastat A, tubastatin A, or HPOB.

8. The method of claim 1, wherein the HDAC6 inhibitor is tubastatin A.

9. The method of claim 1, wherein the HDAC6 inhibitor is ricolinostat.

10. The method of claim 1, wherein the HDAC6 inhibitor is CAY10603.

11. The method of claim 1, wherein the HDAC6 inhibitor is nexturastat A.

12. The method of claim 1, wherein the HDAC6 inhibitor is at least 100-fold selective against HDAC6 compared to all other isozymes of HDAC.

13. The method of any one of claims 1-12, wherein the dilated cardiomyopathy is familial dilated cardiomyopathy.

14. The method of any one of claims 1-12, wherein the dilated cardiomyopathy is dilated cardiomyopathy due to one or more BLC2-Associated Athanogene 3 (BAG3) mutations.

15. The method of any one of claims 1-12, wherein the subject has a deleterious mutation in the BAG3 gene.

16. The method of any one of claims 1-12, wherein the dilated cardiomyopathy is dilated cardiomyopathy due to one or more muscle LIM protein (MLP) mutations.

17. The method of any one of claims 1-12, wherein the subject has a deleterious mutation in the CSPR3 gene encoding MLP.

18. The method of any one of claims 1-12, wherein the subject is a human.

19. The method of any one of claims 1-12, wherein the method restores the ejection fraction of the subject to at least about the ejection fraction of a subject without dilated cardiomyopathy.

20. The method of any one of claims 1-12, wherein the method increases the ejection fraction of the subject compared to the subject's ejection fraction before treatment.

21. The method of any one of claims 1-12, wherein the method restores the ejection fraction of the subject to at least about 20%, at least about 20%, at least about 30%, at least about 40%, or at least about 50%.

22. The method of any one of claims 1-12, wherein the method increase the ejection fraction of the subject to by at least about 5%, at least about 10%, at least about 20%, at least about 30%, or at least about 40%.

23. The method of any one of claims 1-12, wherein the method reduces HDAC6 activity in the heart of the subject.

24. The method of any one of claims 1-12, wherein the method prevents heart failure in the subject.

25. The method of any one of claims 1-12, wherein the method reduces left ventricular internal diameter at diastole (LVIDd) in the subject.

26. The method of any one of claims 1-12, wherein the method reduces left ventricular internal diameter at systole (LVIDs) in the subject.

27. The method of any one of claims 1-12, wherein the method reduces left ventricular mass in the subject.

28. The method of any one of claims 1-12, wherein the method comprises selecting the HDAC6 inhibitor by performing in vitro testing for selective inhibition of HDAC6 on each member of the plurality of candidate compounds, thereby identifying a selected compound for use as the HDAC6 inhibitor.

29. An HDAC6 inhibitor for use in a method for treating dilated cardiomyopathy.

30. A pharmaceutical composition for use in a method for treating dilated cardiomyopathy, comprising an HDAC6 inhibitor.

31. A kit, comprising an HDAC6 inhibitor and instructions for use in a method for treating dilated cardiomyopathy.

32. Use of an HDAC6 inhibitor in treating dilated cardiomyopathy.

33. A method of identifying a compound for treatment of dilated cardiomyopathy, comprising contacting a cell culture comprising cells having an inactivating mutation in BAG3 with each member of a plurality of candidate compounds; and selecting a compound that reduces sarcomere damage in the cells.

34. A method of treating dilated cardiomyopathy in a subject in need thereof, comprising:
a) identifying a compound by contacting a cell culture comprising cells having an inactivating mutation in BAG3 with each member of a plurality of candidate compounds; and selecting a selected compound as reducing sarcomere damage; and
b) administering a therapeutically effective amount of the selected compound to the subject.
